(19) United States
(12) Patent Application Publication
(10) Pub. No.: US 2025/0236682 A1
(43) Pub. Date: Jul. 24, 2025

(54) ANTIGEN-BINDING PROTEIN COMPRISING TWO FC DOMAINS AND USE THEREOF

(71) Applicant: CENTENAIRE BIOSCIENCES, INC., Seongnam-si, Gyeonggi-do (KR)

(72) Inventors: Eun Shik CHOI, Yongin-si, Gyeonggi-do (KR); Hyunkyu PARK, Yongin-si, Gyeonggi-do (KR); Ji Seon BAE, Suwon-si, Gyeonggi-do (KR); Gi-Hyeok YANG, Seoul (KR)

(21) Appl. No.: 18/703,321

(22) PCT Filed: Oct. 22, 2022
(86) PCT No.: PCT/KR2022/015981

§ 371 Date: Apr. 19, 2024

(30) Foreign Application Priority Data
Oct. 22, 2021 (KR) 10-2021-0142135

(51) Int. Cl. C07K 16/32; A61P 35/00; C07K 16/28; C07K 16/30

(52) U.S. Cl. CPC C07K 16/32; A61P 35/00; C07K 16/2803; C07K 16/2863; C07K 16/303; C07K 2317/522; C07K 2317/524; C07K 2317/526; C07K 2317/528; C07K 2317/53; C07K 2317/565; C07K 2317/734; C07K 2317/92

(57) ABSTRACT

The present invention provides a fusion protein comprising one antigen-binding site and two Fc domains and having a novel antibody structure. Although such a novel antibody has a molecular weight similar to human IgG, the antibody structure enables Fc domains to be present on a cell surface antigen a maximum of four times compared to a natural human antibody. Thus, the fusion protein has increased affinity for Fcγ receptors, and has increased effector functions. Therefore, the fusion protein having a novel antibody format can be used as a novel antibody platform.

### TECHNICAL FIELD

The present invention relates to a novel antibody format having an antigen-binding site that specifically binds to a cancer surface antigen, and two Fc domains.

### BACKGROUND ART

Antibody-based therapeutic agents and Fc fusion proteins are a group of clinically important drugs for patients with cancer, immune diseases, infectious diseases, and inflammatory diseases. ADCC (antibody-dependent cell-mediated cytotoxicity), ADCP (antibody-dependent cellular phagocytosis), and CDC (complement-dependent cytotoxicity), which are induced by the interaction between the antibody Fc domain and the innate immune system, play an important role in alleviating or treating symptoms of the disease.

Attempts are being made to maintain the bivalency of the antibody and to improve the effector function by increasing the number of Fc domains (Claudio Sustmann et al., MAbs. 2019; Dennis R Goulet et al., Proteins, 2020). Although these platforms confirmed improvements in binding ability to Fcγ receptors and ADCC, it is difficult to obtain homogeneous antibodies due to the complexity of production and purification. Attempts to improve the effector function by connecting the Fc domains of antibodies in tandem or constituting a large number of Fc domains are also currently underway (U.S. Patent Publication US 2020/0040084 A1). In this case, there is a disadvantage that permeability to the tissue may be significantly lowered due to the increase in the size or molecular weight of the antibody.

### DETAILED DESCRIPTION OF INVENTION

### Technical Problem

Accordingly, the present inventors studied to improve the function of the antibody and at the same time to solve the problems of the existing antibody format designed to comprise multiple Fc domains in tandem as described above. As a result, the present inventors developed a novel improved antibody format that enables Fc domains to be present on a cell surface antigen a maximum of four times compared to a natural human antibody, even though it has a molecular weight (approximately 150 kDa) similar to that of natural human immunoglobulin G (IgG).

### Solution to Problem

In one aspect of the present invention, there is provided a fusion protein comprising an antigen-binding site, a first Fc domain or a variant thereof linked to a first linking position of the antigen-binding site, and a second Fc domain or a variant thereof linked to a second linking position of the antigen-binding site.

In another aspect of the present invention, there is provided a fusion protein comprising two antigen-binding sites linked in tandem (tandem two antigen-binding regions), a first Fc domain or a variant thereof linked to a first linking position of the tandem 2 antigen-binding sites, and a second Fc domain or a variant thereof linked to a second linking position of the tandem 2 antigen-binding sites. According to one embodiment, each of the two antigen-binding sites constituting the tandem 2 antigen-binding sites may be a sequence comprising a CDR sequence or a variable region that is each capable of binding to different epitopes of the same antigen or to different antigens, or a sequence consisting of a variable region.

In the fusion proteins described in the present disclosure, according to one embodiment, the antigen-binding site may be a sequence comprising a CDR sequence or a variable region of an antibody or a sequence consisting of a variable region. Therefore, the antigen-binding site may comprise a first peptide consisting of or comprising a light chain CDR sequence or a light chain variable region of an antibody, and a second peptide consisting of or comprising a heavy chain CDR sequence or a heavy chain variable region of an antibody. The first Fc domain and the second Fc domain may each be a dimer consisting of two peptide sequences. The first peptide of the antigen-binding site binds to a first Fc domain or a variant thereof, and the second peptide of the antigen-binding site binds to a second Fc domain or a variant thereof.

In the fusion proteins described in the present disclosure, according to another embodiment, the first Fc domain and the second Fc domain may be linked to each other through a covalent bond, non-covalent bond, or linker, or may not be linked to each other. In a preferred embodiment, the first Fc domain and the second Fc domain are not linked to each other.

According to embodiments of the present disclosure, the Fc domain may be an Fc domain of a wild-type immunoglobulin, and may comprise modifications for modulating the reactivity of the Fc domain to Fcγ receptors (FcγRs), ADCC or minimizing the formation of undesirable multimers of the Fc domain, for example amino acid substitutions. The Fc domain comprises CH2 and CH3 regions, and may comprise a CH4 region and/or a hinge region, and it should be interpreted that the Fc domain comprises Fc domain fragments that exhibit the function of the Fc domain.

According to embodiments of the present disclosure, an antigen-binding site and an Fc domain or a variant thereof may be joined either directly or through a linker. For example, an antigen-binding site and an Fc domain or a variant thereof may be connected with or without a linker between the N-terminus and the C-terminus, between the N-terminus and the N-terminus, or between the C-terminus and the C-terminus of each peptide molecule.

According to embodiments of the present disclosure, when an antigen-binding site and an Fc domain or a variant thereof is joined through a linker, the linker may be a commonly used pepetide linker. For example, the linker may be a peptide consisting of 1-70 amino acid residues, 2-60 amino acid residues, 2-50 amino acid residues, 2-40 amino acid residues, 2-30 amino acid residues, 3-50 amino acid residues, 3-40 amino acid residues, 3-30 amino acid residues, 2-28 amino acid residues, 2-26 amino acid residues, 2-24 amino acid residues, 2-22 amino acid residues, 2-20 amino acid residues, 2-18 amino acid residues, 2-16 amino acid residues, 2-14 amino acid residues, 2-12 amino acid residues, or 2-10 amino acid residues. The connection between a first Fc domain and an antigen-binding site, the connection between a second Fc domain and an antigen-binding site, or both may be achieved through a linker.

In another aspect of the present invention, there is provided a pharmaceutical composition for preventing or treating cancer, comprising the fusion protein as an active ingredient.

In another aspect of the present invention, there is provided nucleotides encoding the fusion protein, and a vector comprising the nucleotides, and a transformed cell into which the vector has been introduced.

In another aspect of the present invention, there is provided a method for treating or preventing cancer, comprising administering the fusion protein to a subject.

In another aspect of the present invention, there is provided a use of the fusion protein for the treatment of cancer.

### Effects of Invention

Unlike wild type antibodies, the fusion protein having the novel antibody format of the present invention comprises one or two antigen-binding sites and two Fc domains. The two Fc domains are not directly linked to each other, but the two Fc domains are each independently linked to two different polypeptide chains constituting the antigen-binding site. Even though this novel antibody format has a size and molecular weight similar to that of human IgG, it can enable Fc domains to be present on a cell surface antigen a maximum of four times compared to a natural human antibody. Due to these properties, the fusion protein having the novel antibody format has improved affinity (avidity) for Fcγ receptors and can induce improved effector functions. Therefore, the fusion protein having the novel antibody format can be utilized for various purposes by replacing conventional antibodies.

### BRIEF DESCRIPTION OF DRAWINGS

FIG. 1a is a schematic diagram of natural human immunoglobulin (IgG).

FIG. 1b is a schematic diagram of the novel engineered antibody format.

FIG. 1c is a schematic diagram showing a cancer cell with its tumor antigens bound to the antigen-specific human immunoglobulins (IgG) in a monovalent manner.

FIG. 1d is a schematic diagram showing a cancer cell with its tumor antigens bound to the antigen-specific human immunoglobulin (IgG) in a monovalent or bivalent manner.

FIG. 1e is a schematic diagram showing a cancer cell with its tumor antigens bound to the antigen-specific human immunoglobulins (IgG) in a bivalent manner.

FIG. 1f is a schematic diagram showing a cancer cell with its tumor antigens bound to the antigen-specific novel engineered antibody.

FIG. 2a is a schematic diagram of the novel monovalent antibody format (WT) with two Fc domains.

FIG. 2b is a schematic diagram of an antibody (M1) in which VH Q105C and VL A43C amino acid substitutions are introduced in the novel monovalent antibody format with two Fc domains.

FIG. 2c is a schematic diagram of an antibody (M2) in which CH1 F122C and CL S121C amino acid substitutions are introduced in the novel monovalent antibody format with two Fc domains.

FIG. 2d is a schematic diagram of an antibody (M3) in which VH G44C and VL Q100C amino acid substitutions are introduced in the novel monovalent antibody format with two Fc domains.

FIG. 3a illustrates sequence information indicating the position of Cys substitution in the VH-CH1 domain of trastuzumab. The WT sequence is SEQ ID NO: 8, the sequence of Mutant 1 is SEQ ID NO: 10, the sequence of Mutant 2 is SEQ ID NO: 12, and the sequence of Mutant 3 is SEQ ID NO: 14.

FIG. 3b illustrates sequence information indicating the position of Cys substitution in the VL-CL domain of trastuzumab. The WT sequence is SEQ ID NO: 9, the sequence of Mutant 1 is SEQ ID NO: 11, the sequence of Mutant 2 is SEQ ID NO: 13, and the sequence of Mutant 3 is SEQ ID NO: 15.

FIG. 4 illustrates a result obtained by SDS-PAGE analysis of WT, M1, M2, and M3.

FIG. 5a illustrates a result obtained by size exclusion chromatography analysis of WT.

FIG. 5b illustrates a result obtained by size exclusion chromatography analysis of M1.

FIG. 5c illustrates a result obtained by size exclusion chromatography analysis of M2.

FIG. 5d illustrates a result obtained by size exclusion chromatography analysis of M3.

FIG. 6a is a schematic diagram of an antibody (M3) in which the CL domain and the hinge region are linked with a 15-mer peptide.

FIG. 6b is a schematic diagram of an antibody (V1) in which the CL domain and the hinge region are linked with a 10-mer peptide.

FIG. 6c is a schematic diagram of an antibody (V2) in which the CL domain and the hinge region are linked with a 5-mer peptide.

FIG. 6d is a schematic diagram of an antibody (V3) in which the CL domain and the hinge region are directly linked without a linker.

FIG. 7 illustrates a result obtained by SDS-PAGE analysis of M3, V1, V2, and V3.

FIG. 8a is a schematic diagram of fragments generated when H01 and P01 are cleaved with papain.

FIG. 8b is a schematic diagram of fragments generated by papain cleavage of H01 and P01 when a disulfide bond in the hinge region is abnormally formed.

FIG. 8c illustrates a result obtained by SDS-PAGE analysis of the H01 papain cleavage product.

FIG. 8d illustrates a result obtained by SDS-PAGE analysis of the P01 papain cleavage product.

FIG. 9 illustrates a result obtained by SDS-PAGE analysis of H01 wt and H01.

FIG. 10a is a schematic diagram of H01Fv1, which has an Fv-(Fc)₂ structure.

FIG. 10b is a schematic diagram of H01Fv2, which has an Fv-(Fc)₂ structure.

FIG. 10c is a schematic diagram of H01Fv3, which has an Fv-(Fc)₂ structure.

FIG. 10d is a schematic diagram of H01Fv4, which has an Fv-(Fc)₂ structure.

FIG. 10e is a schematic diagram of H01Fv5, which has an Fv-(Fc)₂ structure.

FIG. 10f is a schematic diagram of H01Fv6, which has an Fv-(Fc)₂ structure.

FIG. 10g is a schematic diagram of H01Fv7, which has an Fv-(Fc)₂ structure.

FIG. 10h is a table showing the mutated positions of the Fv-(Fc)₂ structure.

FIG. 11 illustrates a result obtained by SDS-PAGE analysis of the Fv-(Fc)₂ structure after Protein A purification.

FIGS. 12a to 12g illustrate results obtained by SEC analysis of the Fv-(Fc)₂ structure after Protein A purification.

FIG. 13a illustrates an analysis of the sensorgram data for the binding of H01Fv1, a purified Fv-(Fc)₂ structure, to human HER2.

FIG. 13b illustrates an analysis of the sensorgram data for the binding of H01Fv2, a purified Fv-(Fc)₂ structure, to human HER2.

FIG. 13c illustrates an analysis of the sensorgram data for the binding of H01Fv4, a purified Fv-(Fc)₂ structure, to human HER2.

FIG. 13d illustrates an analysis of the sensorgram data for the binding of H01Fv5, a purified Fv-(Fc)₂ structure, to human HER2.

FIG. 13e illustrates an analysis of the sensorgram data for the binding of H01Fv6, a purified Fv-(Fc)₂ structure, to human HER2.

FIG. 13f illustrates an analysis of the sensorgram data for the binding of H01Fv7, a purified Fv-(Fc)₂ structure, to human HER2.

FIG. 14 illustrates a differential scanning fluorimetry analysis of the melting temperatures of H01, P01, trastuzumab, and pertuzumab.

FIG. 15 is a bio-layer interferometry analysis of the sensorgram data showing competitive binding of H01 and P01.

FIG. 16a is a schematic diagram showing the binding mode of H01 in combination with P01 to HER2 on HER2-positive cancer cells.

FIG. 16b is a schematic diagram showing the binding mode of trastuzumab in combination with pertuzumab to HER2 on HER2-positive cancer cells.

FIG. 17a illustrates a flow cytometry analysis of the amount of Fc domain present on the surface of NCI-N87 gastric cancer cell line upon treatment with 50 nM anti-HER2 antibody.

FIG. 17b illustrates a flow cytometry analysis of the amount of Fc domain present on the surface of BT474 breast cancer cell line upon treatment with 50 nM anti-HER2 antibody.

FIG. 17c illustrates a flow cytometry analysis of the amount of Fc domain present on the surface of SK-0V3 ovarian cancer cell line upon treatment with 50 nM anti-HER2 antibody.

FIG. 17d illustrates a flow cytometry analysis of the amount of Fc domain present on the surface of SNU-1 gastric cancer cell line upon treatment with 50 nM anti-HER2 antibody.

FIG. 17e illustrates a flow cytometry analysis of the amount of Fc domain present on the surface of SNU-5 gastric cancer cell line upon treatment with 50 nM anti-HER2 antibody.

FIG. 18a illustrates a flow cytometry analysis of the amount of Fc domain present on the surface of NCI-N87 gastric cancer cell line upon treatment with anti-HER2 antibody at indicated concentrations.

FIG. 18b illustrates a flow cytometry analysis of the amount of Fc domain present on the surface of BT474 breast cancer cell line upon treatment with anti-HER2 antibody at indicated concentrations.

FIG. 18c illustrates a flow cytometry analysis of the amount of Fc domain present on the surface of SK-0V3 ovarian cancer cell line upon treatment with anti-HER2 antibody at indicated concentrations.

FIG. 18d illustrates a flow cytometry analysis of the amount of Fc domain present on the surface of SNU-1 gastric cancer cell line upon treatment with anti-HER2 antibody at indicated concentrations.

FIG. 18e illustrates a flow cytometry analysis of the amount of Fc domain present on the surface of SNU-5 gastric cancer cell line upon treatment with anti-HER2 antibody at indicated concentrations.

FIG. 19a is a schematic diagram of H01DE4 in which S239D and 1332E mutations were introduced in H01.

FIG. 19b is a schematic diagram of P01DE4 in which S239D and 1332E mutations were introduced in P01.

FIGS. 20a to 20d are sensorgrams binding profiles of H01, H01DE4, P01, and P01DE4 for human HER2.

FIGS. 21a to 21h are sensorgrams binding profiles of H01, P01, H01DE4, P01DE4, human IgG1, trastuzumab, pertuzumab, and margetuximab for Fcγ receptor 1.

FIGS. 22a to 22h are sensorgrams binding profiles of H01, P01, H01DE4, P01DE4, human IgG1, trastuzumab, pertuzumab, and margetuximab for Fcγ receptor 2A (131R isoform).

FIGS. 23a to 23h are sensorgrams binding profiles of H01, P01, H01DE4, P01DE4, human IgG1, trastuzumab, pertuzumab, and margetuximab for Fcγ receptor 3A (176V isoform).

FIG. 24a is a graph showing an antibody concentration in blood over time when H01, P01, trastuzumab, and pertuzumab were administered intravenously at 10 mg/kg to Sprague-Dawley rats.

FIG. 24b illustrates the PK parameters calculated from FIG. 24a when H01, P01, trastuzumab, and pertuzumab were administered intravenously at 10 mg/kg to Sprague-Dawley rats.

FIG. 25 is a schematic diagram of HP501, which is a HER2 biparatopic engineered antibody.

FIG. 26 is a schematic diagram of HP501 to HP516, which are HER2 biparatopic engineered antibodies.

FIG. 27 illustrates a size exclusion chromatography analysis of HP501 to HP516, which are HER2 biparatopic engineered antibodies.

FIG. 28a is a bio-layer interferometry analysis of the sensorgram data for the binding of HP503 to HER2.

FIG. 28b is a bio-layer interferometry analysis of the sensorgram data for the binding of HP507 to HER2.

FIG. 28c is a bio-layer interferometry analysis of the sensorgram data for the binding of HP511 for HER2.

FIG. 28d is a bio-layer interferometry analysis of the sensorgram data for the binding of HP515 for HER2 protein using bio-layer interferometry analysis.

FIG. 29a is a bio-layer interferometry analysis of the sensorgram data for the binding of HP503 for Fcγ receptor 1, Fcγ receptor 2A (131R isoform), and Fcγ receptor 3A (176V isoform).

FIG. 29b is a bio-layer interferometry analysis of the sensorgram data for the binding of HP507 for Fcγ receptor 1, Fcγ receptor 2A (131R isoform), and Fcγ receptor 3A (176V isoform).

FIG. 29c is a bio-layer interferometry analysis of the sensorgram data for the binding of HP511 for Fcγ receptor 1, Fcγ receptor 2A (131R isoform), and Fcγ receptor 3A (176V isoform).

FIG. 29d is a bio-layer interferometry analysis of the sensorgram data for the binding of HP515 for Fcγ receptor 1, Fcγ receptor 2A (131R isoform), and Fcγ receptor 3A (176V isoform).

FIG. 30a is a bio-layer interferometry analysis of the sensorgram data for the binding of HP503, HP507, HP511, and HP515 for the neonatal Fc receptor (FcRn).

FIG. 30b is a bio-layer interferometry analysis of the sensorgram data for the binding of human IgG1, trastuzumab, pertuzumab, and margetuximab for the neonatal Fc receptor (FcRn).

FIG. 31a illustrates the CDC activity of anti-HER2 antibodies in the BT474 breast cancer cell line.

FIG. 31b illustrates the CDC activity of anti-HER2 antibodies in the NCI-N87 gastric cancer cell line.

FIG. 32a illustrates the ADCC activity of anti-HER2 antibodies in the NCI-N87 gastric cancer cell line.

FIG. 32b illustrates the ADCC activity of anti-HER2 antibodies in the MDA-MB-453 breast cancer cell line.

FIG. 32c illustrates the ADCC activity of anti-HER2 antibodies in the SNU-601 gastric cancer cell line.

FIG. 32d illustrates the ADCC activity of anti-HER2 antibodies in the SNU-5 gastric cancer cell line.

FIG. 33a illustrates the antitumor activity of anti-HER2 antibodies in a C.B-17 SCID mouse model of tumor xenograft of SNU-5 gastric cancer cell line.

FIG. 33b illustrates the antitumor activity of anti-HER2 antibodies in a Balb/c-nude mouse model of tumor xenograft of SNU-5 gastric cancer cell line.

FIG. 34 illustrates the antitumor activity of anti-HER2 antibodies in a mouse model of tumor xenograft of SNU-601 gastric cancer cell line.

FIG. 35 illustrates the antitumor activity of anti-HER2 antibodies in a mouse model of tumor xenograft of NCI-N87 gastric cancer cell line.

FIG. 36 illustrates a vector to express human HER2 protein in mammalian cells.

FIG. 37 illustrates the flow cytometry quantification of HER2 expression in a CT26 mouse large intestine cancer cell line clone expressing human HER2(CT26-HER2).

FIG. 38 illustrates the stability of human HER2 expression in the CT26-HER2 clones.

FIG. 39 illustrates the relative expression of human HER2 in CT26-HER2 cell line (Clone name: #2-60) compared to human cancer cell lines, and shows that H01 allows a greater amount of Fc domain to bind to the surface of CT26-HER2 cells compared to trastuzumab.

FIG. 40 illustrates antitumor activity of anti-HER2 antibodies in a syngeneic CT26-HER2 mouse tumor model.

FIG. 41a is a schematic diagram of a monovalent engineered mAb according to one embodiment.

FIG. 41b is a schematic diagram of a biparatopic engineered mAb according to one embodiment.

FIG. 42 illustrates the bio-layer interferometry analysis of the sensorgram data for the binding of a fusion protein to the target according to one embodiment. Specifically, FIG. 42a illustrates the sensorgram data for the binding of GPM01, a monovalent engineered mAb targeting GPC-3, to human GPC-3. FIG. 42b illustrates the sensorgram data for the binding of GPM02, a monovalent engineered mAb targeting GPC-3, to human GPC-3. FIG. 42c illustrates the sensorgram data for the binding of GPM04, a monovalent engineered mAb targeting GPC-3, to human GPC-3. FIG. 42d illustrates the sensorgram data for the binding of GPB01, a biparatopic engineered mAb targeting GPC-3, to human GPC-3. FIG. 42e illustrates the sensorgram data for the binding of GPB03, a biparatopic engineered mAb targeting GPC-3, to human GPC-3. FIG. 42f illustrates the sensorgram data for the binding of GPB04, a biparatopic engineered mAb targeting GPC-3, to human GPC-3. FIG. 42g illustrates the sensorgram data for the binding of GPB06, a biparatopic engineered mAb targeting GPC-3, to human GPC-3.

FIG. 43 illustrates the flow cytometry analysis of the amount of Fc domain present on the surface of HepG2 liver cancer cell line upon treatment with 100 nM GPC-3 antibody.

FIG. 44 illustrates the SDS-PAGE analysis showing the inhibition of AKT phosphorylation in the PC-3 prostate cancer cell line upon treatment with 50 nM EphA2 antibody.

FIG. 45 illustrates the flow cytometry analysis of the amount of Fc domain present on the surface of PC-3 prostate cancer cell line upon treatment with 100 nM EphA2 antibody.

FIG. 46 illustrates the bio-layer interferometry analysis of the sensorgram data for the binding of MEM01 and MEM06, which are monovalent engineered mAbs targeting MET, to human MET.

FIG. 47 illustrates the flow cytometry analysis of the amount of Fc domain present on the surface of MKN45 gastric cancer cell line upon treatment with MET antibody at indicated concentrations.

FIG. 48 illustrates the flow cytometry analysis of the amount of Fc domain present on the surface of SNU5 gastric cancer cell line upon treatment with an antibody at indicated concentrations.

FIG. 49 illustrates the bio-layer interferometry analysis of the sensorgram data for the binding of monovalent engineered mAbs targeting EGFR to human EGFR.

FIG. 50 illustrates the bio-layer interferometry analysis of the sensorgram data for the binding of 33-1, 33-2, and 33-3, which are monovalent engineered mAbs targeting CD33, and 33-4, 33-5, 33-6, and 33-7, which are biparatopic engineered mAbs targeting CD33, to human CD33.

FIG. 51 illustrates the bio-layer interferometry analysis of the sensorgram data for the binding of monovalent engineered mAbs targeting CEACAM5 to human CEACAM5.

FIG. 52 illustrates the bio-layer interferometry analysis of the sensorgram data for the binding of a fusion protein according to one embodiment for the target. Specifically, FIG. 52a illustrates the sensorgram data for the binding of T01, a monovalent engineered mAb targeting TROP2, to human TROP2. FIG. 52b illustrates the sensorgram data for the binding of MSM01, a monovalent engineered mAb targeting mesothelin, to human mesothelin. FIG. 52c illustrates the sensorgram data for the binding of LIM01, a monovalent engineered mAb targeting LIV-1, to human LIV-1. A:T01, B:MSM01, C:LIM01.

### BEST MODE FOR CARRYING OUT THE INVENTION

### Definition of Terms

As used herein, the term “fusion protein with two Fcs” or “antibody with two Fcs” refers to a fusion protein in which two Fc domains are independently joined to two polypeptide chains constituting the antigen-binding site. The two polypeptide chains constituting the antigen-binding site may be different from each other. For example, one of the two polypeptide chains constituting the antigen-binding site may be a sequence comprising or consisting of the light chain CDR sequence or light chain variable region of the antibody, or may be an scFv, and the other may be a sequence comprising or consisting of the heavy chain CDR sequence or heavy chain variable region of the antibody, or may be an scFv. In one embodiment, the fusion protein having the two Fc regions may comprise the sequence of a “humanized” form of a non-human antibody, which is a chimeric antibody comprising a human immunoglobulin comprising native CDRs. In addition, the fusion protein may comprise a “fully human antibody” or a portion of a “human antibody.” In addition, in one embodiment, the multispecific fusion protein or antigen binding domain may be a “monoclonal antibody” or a portion thereof.

As used herein, the term “antibody” refers to a substance that specifically binds to an antigen and causes an antigen-antibody reaction. In addition, the antibody is also referred to as immunoglobulin. The antibody may refer to any one selected from IgG, IgE, IgM, IgD, and IgA, and may be a subclass of IgG, such as IgG1, IgG2, IgG3, IgG4, IgA1, or IgA2. In addition, the antibody may be an agonistic antibody or an antagonistic antibody.

As used herein, the term “Fab” or “Fab region” refers to a region of an antibody that binds to an antigen. Conventional IgG generally comprises two Fab regions. Each Fab region typically consists of one variable region and one constant region of each heavy chain and light chain. Specifically, the variable region and the constant region of the heavy chain in the Fab region are the VH and CH1 regions, and the variable region and the constant region of the light chain in the Fab region are the VL and CL regions. VH, CH1, VL, and CL of the Fab region may be arranged in various ways to impart the antigen binding ability according to the present disclosure, including the CrossMab Fab technology in which VH and VL have an arrangement substituted for each other.

As used herein, the term “heavy chain” refers to a polypeptide chain of about 50 kDa to about 70 kDa. Here, the N-terminal portion comprises a variable region of at least about 120 to 130 amino acids, and the C-terminal portion comprises a constant region. The constant region may be one of five types: alpha (α), delta (δ), epsilon (ε), gamma (γ), and mu (μ). Here, α, δ, and γ comprise about 450 amino acids, and μ and ε comprise about 550 amino acids.

As used herein, the term “light chain” refers to a polypeptide chain of about 25 kDa. Here, the N-terminal portion comprises a variable region of at least about 100 to about 110 amino acids, and the C-terminal portion comprises a constant region. There are two types of light chain constant domains: kappa (κ) or lambda (λ). In addition, the constant region of the light chain is referred to as “CL”. The heavy chain C domains (CH domains) are numbered from N-terminus to C-terminus (e.g., CH1, CH2, CH3, etc.). The CL and a CH1 regions of any of these antibody classes may be used in the present disclosure. In certain embodiments, the CL and CH1 regions provided herein are of the IgG type (for example, IgG1).

As used herein, the term “Fc” or “Fc region” refers to the C-terminal region of an immunoglobulin heavy chain, including a native Fc region, a recombinant Fc region, and a variant Fc region. Therefore, Fc refers to the last two constant region immunoglobulin domains of IgA, IgD, and IgG, the last three constant region immunoglobulin domains of IgE and IgM, and the hinge present in the N-terminus of these domains. For IgA and IgM, the Fc may comprise a J chain. For IgG, the Fc comprises the immunoglobulin domains Cy2 (CH2) and Cy3 (CH3) and the hinge between Cy1 and Cy2. Although the interface of the Fc region may vary, the human IgG heavy chain Fc region is generally defined as comprising residues C226, P230, or A231 at the C-terminus, where numbering is according to the EU index. As used herein, “Fc polypeptide” or “Fc-derived polypeptide” refers to a polypeptide comprising all or part of an Fc region. In one embodiment, a variant Fc region may be in a form in which at least one amino acid, for example, about 1 to about 10 amino acids, or about 1 to about 5 amino acids, are substituted compared to the native sequence Fc region. In addition, the variant Fc region may have at least about 80% homology, at least about 90% homology, or at least about 95% homology to the native sequence Fc region.

As used herein, the term “Fv” or “Fv fragment” or “Fv region” is a polypeptide comprising the VL and VH domains of a single antibody.

As used herein, the term “single chain Fv” or “scFv” refers to an antibody fragment comprising the VH and VL domains of an antibody within a single polypeptide chain.

As used herein, the term “variable region” refers to an antibody region comprising one or more immunoglobulin domains encoded by any one of the VL (including Vkappa (VK) and Vlambda (VL)) and/or VH genes that constitute the light chain (including kappa and lambda) and heavy chain immunoglobulin loci, respectively. The light or heavy chain variable region (VL or VH) consists of a “framework” or “FR” region that includes three hypervariable regions referred to as “complementarity determining regions” or “CDRs”. As used herein, the term “antigen” refers to a structure capable of selectively binding to an antibody. A target antigen may be a polypeptide, carbohydrate, nucleic acid, lipid, hapten, or other naturally occurring compound or synthetic compound. Specifically, an antigen is a polypeptide and may be a protein present on or within a cell.

As used herein, the term “epitope” refers to an antigenic determinant and is a part on an antigen to which an antibody or polypeptide binds. A protein epitope may comprise amino acid residues that are directly involved in binding as well as amino acid residues that are effectively blocked by specific antigen-binding antibodies or peptides. It is the simplest form or smallest structural region of a complex antigen molecule that may bind to an antibody or receptor. The epitope may be linear or structural/conformational.

As used herein, the term “vector” refers to a material for transporting or expressing a nucleic acid sequence including a nucleic acid sequence encoding a multispecific fusion protein (for example, an antibody) described herein. Specifically, vectors include expression vectors, plasmids, phage vectors, viral vectors, episomes, and artificial chromosomes.

As used herein, the term “polynucleotide,” also referred to as “nucleic acid,” refers to a polymer of nucleotides of any length. Specifically, the polynucleotide may be DNA or RNA.

### Antibody Comprising Two Fcs

In one aspect of the present invention, there is provided an antibody comprising a plurality of Fc domains, characterized in that the ratio of the antigen-binding site and the Fc domain is 1:2 or 2:2. Specifically, the antibody may be a fusion protein comprising an antigen-binding site, a first Fc domain or a variant thereof, and a second Fc domain or a variant thereof.

Here, the antigen-binding site may consist of two different polypeptide chains. In addition, each polypeptide may be linked to a first Fc domain or a variant thereof, and a second Fc domain or a variant thereof.

Here, in one embodiment of the fusion protein, when the antigen-binding site comprises Fab, it may be a fusion protein in which the two Fc domains are bound to the C-terminus of the CH1 region of the heavy chain and the C-terminus of the constant region of the light chain, respectively. In addition, the Fc domain and Fab may be linked through a peptide linker.

In addition, in one embodiment of the fusion protein, when the antigen-binding site is Fv, it may be a fusion protein in which the two Fc domains are bound to the C-terminus of the variable region of the heavy chain and the C-terminus of the variable region of the light chain, respectively. In addition, the Fc domain and Fv may be linked through a peptide linker.

This novel antibody format or structure has a molecular weight similar to human IgG. In addition, the fusion protein may have an antigen binding affinity equivalent to that of a human IgG-based antibody. However, the antibody format enables Fc domains to be present on a cell surface antigen a maximum of four times compared to a natural human antibody. Due to these characteristics, the fusion protein may have increased affinity for Fcγ receptors, and may have increased effector functions compared to the wild type antibody. Each Fc domain bound to the fusion protein may have a similar level of Fc receptor (Fcγ receptor and FcRn) binding affinity as the Fc domain of an IgG-based antibody, but due to the avidity effect, the apparent binding affinity (apparent affinity) of the fusion protein to the Fc receptor (Fcγ receptor and FcRn) may be significantly increased compared to a human IgG antibody. In addition, the fusion protein has a similar level of thermal stability as that of an IgG-based antibody.

Specifically, the fusion protein may be a fusion protein comprising (a) an antigen-binding site consisting of a first polypeptide comprising at least one complementarity-determining region (CDR) sequence and a second polypeptide comprising at least one complementarity-determining region (CDR) sequence, wherein the first polypeptide and the second polypeptide form a dimer, and the antigen-binding site is capable of specifically binding to a target antigen, (b) a first Fc domain or a variant thereof that is a dimer consisting of two polypeptide sequences, one of which is joined to the first polypeptide of the antigen-binding site, and (c) a second Fc domain or a variant thereof that is a dimer consisting of two polypeptide sequences, one of which is joined to the second polypeptide of the antigen-binding site.

Here, the first polypeptide of the antigen-binding site may comprise CDR1, CDR2, and CDR3 of an antibody heavy chain, and the second polypeptide of the antigen-binding site may comprise CDR1, CDR2, and CDR3 of an antibody light chain. In addition, the first polypeptide of the antigen-binding site may further comprise a CH1 region of an antibody heavy chain, and/or the second polypeptide of the antigen-binding site may further comprise a constant region of an antibody light chain.

The specific structure of the fusion protein is described in more detail below.

### Antigen-Binding Site

Here, the antigen-binding site is capable of specifically binding to a protein expressed on the cell surface. Specifically, the antigen-binding site is capable of specifically binding to a cancer antigen.

In one embodiment, the antigen-binding site is capable of specifically binding to any one selected from the group consisting of PD-L1, EGFR, EGFRvIII, BCMA, CD22, CD25, CD30, CD33, CD37, CD38, CD52, CD56, CD123, c-Met (MET), DLL3, DR4, DR5, GD2, nectin-4, RANKL, SLAMF7, Trop-2, LIV-1, claudin 18.2, IL13α2, CD3, HER2, HER3, FGFR2, FGFR3, GPC3, ROR1, Folα, CD20, CD19, CTLA-4, VEGFR, NCAM1, ICAM-1, ICAM-2, CEACAM5, CEACAM6, carcinoembryonic antigen (CEA), CA-125, alphafetoprotein (AFP), MUC-1, MUC-16, PSMA, PSCA, epithelial tumor antigen (ETA), melanoma-associated antigen (MAGE), immature laminin receptor, TAG-72, HPV E6/E7, BING-4, calcium-activated chloride channel 2, cyclin-B1, 9D7, Ep-CAM, EphA2, EphA3, mesothelin, SAP-1, survivin, and virus-derived antigens.

A second antigen-binding site is also capable of specifically binding to any one antigen selected from the above group. According to one embodiment, the antigen to which a first antigen-binding site binds may be different from the antigen to which a second antigen-binding site binds. For example, the first antigen-binding site may comprise a sequence that specifically binds to HER2, and the second antigen-binding site may comprise a sequence that specifically binds to EGFR. In another embodiment, the first antigen-binding site may comprise a sequence that specifically binds to one epitope of an antigen, and the second antigen-binding site may comprise a sequence that specifically binds to a different epitope of the same antigen.

### Specific Examples of Antigen-Binding Sites

Here, the antigen-binding site may comprise a variable region that specifically binds to the antigen. Specifically, the variable region may include the heavy chain variable region and light chain variable region of any one antibody selected from the group consisting of cetuximab, panitumumab, necitumumab, imgatuzumab, depatuxizumab, losatuxizumab, etevritamab, AMG-595, atezolizumab, avelumab, durvalumab, trastuzumab, pertuzumab, onartuzumab, emibetuzumab, telisotuzumab, datopotamab, sacituzumab, rovalpituzumab, tarlatamab, belantamab, ladiratuzumab, codrituzumab, aprutumab, bemarituzumab, vofatamab, ramucirumab, rituximab, obinutuzumab, daratumumab, and 1C1(Clone name), but is not limited thereto.

As a specific example of the present invention, it may include an antigen-binding site that specifically binds to EGFR. For example, it may comprise a heavy chain variable region comprising H-CDR1 represented by SEQ ID NO: 175, H-CDR2 represented by SEQ ID NO: 176, and H-CDR3 represented by SEQ ID NO: 177 of cetuximab, and may comprise a light chain variable region comprising L-CDR1 represented by SEQ ID NO: 178, L-CDR2 represented by SEQ ID NO: 179, and L-CDR3 represented by SEQ ID NO: 180. As another example, it may comprise a heavy chain variable region comprising H-CDR1 represented by SEQ ID NO: 181, H-CDR2 represented by SEQ ID NO: 182, and H-CDR3 represented by SEQ ID NO: 183 of panitumumab, and may comprise a light chain variable region comprising L-CDR1 represented by SEQ ID NO: 184, L-CDR2 represented by SEQ ID NO: 185, and L-CDR3 represented by SEQ ID NO: 186. As another example, it may comprise a heavy chain variable region comprising H-CDR1 represented by SEQ ID NO: 187, H-CDR2 represented by SEQ ID NO: 188, and H-CDR3 represented by SEQ ID NO: 189 of necitumumab, and may comprise a light chain variable region comprising L-CDR1 represented by SEQ ID NO: 190, L-CDR2 represented by SEQ ID NO: 191, and L-CDR3 represented by SEQ ID NO: 192. In addition, it may comprise a heavy chain variable region comprising H-CDR1 represented by SEQ ID NO: 193, H-CDR2 represented by SEQ ID NO: 194, and H-CDR3 represented by SEQ ID NO: 195 of imgatuzumab, and may comprise a light chain variable region comprising L-CDR1 represented by SEQ ID NO: 196, L-CDR2 represented by SEQ ID NO: 197, and L-CDR3 represented by SEQ ID NO: 198. In addition, it may comprise a heavy chain variable region comprising H-CDR1 represented by SEQ ID NO: 199, H-CDR2 represented by SEQ ID NO: 200, and H-CDR3 represented by SEQ ID NO: 201 of depatuxizumab, and may comprise a light chain variable region comprising L-CDR1 represented by SEQ ID NO: 202, L-CDR2 represented by SEQ ID NO: 203, and L-CDR3 represented by SEQ ID NO: 204. In addition, it may comprise a heavy chain variable region comprising H-CDR1 represented by SEQ ID NO: 199, H-CDR2 represented by SEQ ID NO: 205, and H-CDR3 represented by SEQ ID NO: 206 of losatuxizumab, and may comprise a light chain variable region comprising L-CDR1 represented by SEQ ID NO: 202, L-CDR2 represented by SEQ ID NO: 203, and L-CDR3 represented by SEQ ID NO: 204.

As a specific example of the present invention, it may include an antigen-binding site that specifically binds to EGFRvIII. For example, it may comprise a heavy chain variable region comprising H-CDR1 represented by SEQ ID NO: 207, H-CDR2 represented by SEQ ID NO: 208, and H-CDR3 represented by SEQ ID NO: 209 of etevritamab, and may comprise a light chain variable region comprising L-CDR1 represented by SEQ ID NO: 210, L-CDR2 represented by SEQ ID NO: 211, and L-CDR3 represented by SEQ ID NO: 212. In addition, it may comprise a heavy chain variable region comprising H-CDR1 represented by SEQ ID NO: 213, H-CDR2 represented by SEQ ID NO: 214, and H-CDR3 represented by SEQ ID NO: 215 of AMG-595, and may comprise a light chain variable region comprising L-CDR1 represented by SEQ ID NO: 210, L-CDR2 represented by SEQ ID NO: 216, and L-CDR3 represented by SEQ ID NO: 217.

As a specific example of the present invention, it may include an antigen-binding site that specifically binds to PD-L1. For example, it may comprise a heavy chain variable region comprising H-CDR1 represented by SEQ ID NO: 218, H-CDR2 represented by SEQ ID NO: 219, and H-CDR3 represented by SEQ ID NO: 220 of atezolizumab, and may comprise a light chain variable region comprising L-CDR1 represented by SEQ ID NO: 221, L-CDR2 represented by SEQ ID NO: 222, and L-CDR3 represented by SEQ ID NO: 223. In addition, it may comprise a heavy chain variable region comprising H-CDR1 represented by SEQ ID NO: 224, H-CDR2 represented by SEQ ID NO: 225, and H-CDR3 represented by SEQ ID NO: 226 of avelumab, and may comprise a light chain variable region comprising L-CDR1 represented by SEQ ID NO: 227, L-CDR2 represented by SEQ ID NO: 228, and L-CDR3 represented by SEQ ID NO: 229. In addition, it may comprise a heavy chain variable region comprising H-CDR1 represented by SEQ ID NO: 230, H-CDR2 represented by SEQ ID NO: 231, and H-CDR3 represented by SEQ ID NO: 232 of durvalumab, and may comprise a light chain variable region comprising L-CDR1 represented by SEQ ID NO: 233, L-CDR2 represented by SEQ ID NO: 234, and L-CDR3 represented by SEQ ID NO: 235.

As a specific example of the present invention, it may include an antigen-binding site that specifically binds to HER2. For example, it may comprise a heavy chain variable region comprising H-CDR1 represented by SEQ ID NO: 21, H-CDR2 represented by SEQ ID NO: 22, and H-CDR3 represented by SEQ ID NO: 23 of trastuzumab, and may comprise a light chain variable region comprising L-CDR1 represented by SEQ ID NO: 24, L-CDR2 represented by SEQ ID NO: 25, and L-CDR3 represented by SEQ ID NO: 26. In addition, it may comprise a heavy chain variable region comprising H-CDR1 represented by SEQ ID NO: 33, H-CDR2 represented by SEQ ID NO: 34, and H-CDR3 represented by SEQ ID NO: 35 of pertuzumab, and may comprise a light chain variable region comprising L-CDR1 represented by SEQ ID NO: 36, L-CDR2 represented by SEQ ID NO: 37, and L-CDR3 represented by SEQ ID NO: 38.

As a specific example of the present invention, it may include an antigen-binding site that specifically binds to c-Met. For example, it may comprise a heavy chain variable region comprising H-CDR1 represented by SEQ ID NO: 236, H-CDR2 represented by SEQ ID NO: 237, and H-CDR3 represented by SEQ ID NO: 238 of onartuzumab, and may comprise a light chain variable region comprising L-CDR1 represented by SEQ ID NO: 239, L-CDR2 represented by SEQ ID NO: 240, and L-CDR3 represented by SEQ ID NO: 241. In addition, it may comprise a heavy chain variable region comprising H-CDR1 represented by SEQ ID NO: 242, H-CDR2 represented by SEQ ID NO: 243, and H-CDR3 represented by SEQ ID NO: 244 of emibetuzumab, and may comprise a light chain variable region comprising L-CDR1 represented by SEQ ID NO: 245, L-CDR2 represented by SEQ ID NO: 246, and L-CDR3 represented by SEQ ID NO: 247. In addition, it may comprise a heavy chain variable region comprising H-CDR1 represented by SEQ ID NO: 248, H-CDR2 represented by SEQ ID NO: 249, and H-CDR3 represented by SEQ ID NO: 250 of telisotuzumab, and may comprise a light chain variable region comprising L-CDR1 represented by SEQ ID NO: 251, L-CDR2 represented by SEQ ID NO: 252, and L-CDR3 represented by SEQ ID NO: 253.

As a specific example of the present invention, it may include an antigen-binding site that specifically binds to Trop-2. For example, it may comprise a heavy chain variable region comprising H-CDR1 represented by SEQ ID NO: 254, H-CDR2 represented by SEQ ID NO: 255, and H-CDR3 represented by SEQ ID NO: 256 of datopotamab, and may comprise a light chain variable region comprising L-CDR1 represented by SEQ ID NO: 257, L-CDR2 represented by SEQ ID NO: 258, and L-CDR3 represented by SEQ ID NO: 259. In addition, it may comprise a heavy chain variable region comprising H-CDR1 represented by SEQ ID NO: 260, H-CDR2 represented by SEQ ID NO: 261, and H-CDR3 represented by SEQ ID NO: 262 of sacituzumab, and may comprise a light chain variable region comprising L-CDR1 represented by SEQ ID NO: 263, L-CDR2 represented by SEQ ID NO: 264, and L-CDR3 represented by SEQ ID NO: 265.

As a specific example of the present invention, it may include an antigen-binding site that specifically binds to DLL3. For example, it may comprise a heavy chain variable region comprising H-CDR1 represented by SEQ ID NO: 266, H-CDR2 represented by SEQ ID NO: 267, and H-CDR3 represented by SEQ ID NO: 268 of rovalpituzumab, and may comprise a light chain variable region comprising L-CDR1 represented by SEQ ID NO: 269, L-CDR2 represented by SEQ ID NO: 270, and L-CDR3 represented by SEQ ID NO: 271. In addition, it may comprise a heavy chain variable region comprising H-CDR1 represented by SEQ ID NO: 272, H-CDR2 represented by SEQ ID NO: 273, and H-CDR3 represented by SEQ ID NO: 274 of tarlatamab, and may comprise a light chain variable region comprising L-CDR1 represented by SEQ ID NO: 275, L-CDR2 represented by SEQ ID NO: 276, and L-CDR3 represented by SEQ ID NO: 277.

As a specific example of the present invention, it may include an antigen-binding site that specifically binds to BCMA. For example, it may comprise a heavy chain variable region comprising H-CDR1 represented by SEQ ID NO: 278, H-CDR2 represented by SEQ ID NO: 279, and H-CDR3 represented by SEQ ID NO: 280 of belantamab, and may comprise a light chain variable region comprising L-CDR1 represented by SEQ ID NO: 281, L-CDR2 represented by SEQ ID NO: 282, and L-CDR3 represented by SEQ ID NO: 283.

As a specific example of the present invention, it may include an antigen-binding site that specifically binds to LIV-1. For example, it may comprise a heavy chain variable region comprising H-CDR1 represented by SEQ ID NO: 284, H-CDR2 represented by SEQ ID NO: 285, and H-CDR3 represented by SEQ ID NO: 286 of ladiratuzumab, and may comprise a light chain variable region comprising L-CDR1 represented by SEQ ID NO: 287, L-CDR2 represented by SEQ ID NO: 288, and L-CDR3 represented by SEQ ID NO: 289.

As a specific example of the present invention, it may include an antigen-binding site that specifically binds to GPC-3. For example, it may comprise a heavy chain variable region comprising H-CDR1 represented by SEQ ID NO: 99, H-CDR2 represented by SEQ ID NO: 100, and H-CDR3 represented by SEQ ID NO: 101 of codrituzumab, and may comprise a light chain variable region comprising L-CDR1 represented by SEQ ID NO: 102, L-CDR2 represented by SEQ ID NO: 103, and L-CDR3 represented by SEQ ID NO: 104.

As a specific example of the present invention, it may include an antigen-binding site that specifically binds to FGFR2. For example, it may comprise a heavy chain variable region comprising H-CDR1 represented by SEQ ID NO: 290, H-CDR2 represented by SEQ ID NO: 291, and H-CDR3 represented by SEQ ID NO: 292 of aprutumab, and may comprise a light chain variable region comprising L-CDR1 represented by SEQ ID NO: 293, L-CDR2 represented by SEQ ID NO: 294, and L-CDR3 represented by SEQ ID NO: 295. In addition, it may comprise a heavy chain variable region comprising H-CDR1 represented by SEQ ID NO: 296, H-CDR2 represented by SEQ ID NO: 297, and H-CDR3 represented by SEQ ID NO: 298 of bemarituzumab, and may comprise a light chain variable region comprising L-CDR1 represented by SEQ ID NO: 299, L-CDR2 represented by SEQ ID NO: 300, and L-CDR3 represented by SEQ ID NO: 301.

As a specific example of the present invention, it may include an antigen-binding site that specifically binds to FGFR3. For example, it may comprise a heavy chain variable region comprising H-CDR1 represented by SEQ ID NO: 302, H-CDR2 represented by SEQ ID NO: 303, and H-CDR3 represented by SEQ ID NO: 304 of vofatamab, and may comprise a light chain variable region comprising L-CDR1 represented by SEQ ID NO: 305, L-CDR2 represented by SEQ ID NO: 306, and L-CDR3 represented by SEQ ID NO: 307.

As a specific example of the present invention, it may include an antigen-binding site that specifically binds to VEGFR2. For example, it may comprise a heavy chain variable region comprising H-CDR1 represented by SEQ ID NO: 308, H-CDR2 represented by SEQ ID NO: 309, and H-CDR3 represented by SEQ ID NO: 310 of ramucirumab, and may comprise a light chain variable region comprising L-CDR1 represented by SEQ ID NO: 311, L-CDR2 represented by SEQ ID NO: 312, and L-CDR3 represented by SEQ ID NO: 313.

As a specific example of the present invention, it may include an antigen-binding site that specifically binds to CD20. For example, it may comprise a heavy chain variable region comprising H-CDR1 represented by SEQ ID NO: 314, H-CDR2 represented by SEQ ID NO: 315, and H-CDR3 represented by SEQ ID NO: 316 of rituximab, and may comprise a light chain variable region comprising L-CDR1 represented by SEQ ID NO: 317, L-CDR2 represented by SEQ ID NO: 318, and L-CDR3 represented by SEQ ID NO: 319. For example, it may comprise a heavy chain variable region comprising H-CDR1 represented by SEQ ID NO: 320, H-CDR2 represented by SEQ ID NO: 321, and H-CDR3 represented by SEQ ID NO: 322 of obinutuzumab, and may comprise a light chain variable region comprising L-CDR1 represented by SEQ ID NO: 323, L-CDR2 represented by SEQ ID NO: 324, and L-CDR3 represented by SEQ ID NO: 325.

As a specific example of the present invention, it may include an antigen-binding site that specifically binds to CD38. For example, it may comprise a heavy chain variable region comprising H-CDR1 represented by SEQ ID NO: 326, H-CDR2 represented by SEQ ID NO: 327, and H-CDR3 represented by SEQ ID NO: 328 of daratumumab, and may comprise a light chain variable region comprising L-CDR1 represented by SEQ ID NO: 329, L-CDR2 represented by SEQ ID NO: 330, and L-CDR3 represented by SEQ ID NO: 331.

As a specific example of the present invention, it may include an antigen-binding site that specifically binds to EphA2. For example, it may comprise a heavy chain variable region comprising H-CDR1 represented by SEQ ID NO: 157, H-CDR2 represented by SEQ ID NO: 158, and H-CDR3 represented by SEQ ID NO: 159 of 1C1, and may comprise a light chain variable region comprising L-CDR1 represented by SEQ ID NO: 160, L-CDR2 represented by SEQ ID NO: 161, and L-CDR3 represented by SEQ ID NO: 162.

Table 1 below shows the CDR sequences of non-limiting exemplary antibodies with anticancer efficacy that may be used in specific examples of the present invention.

TABLE 1

| | Heavy chain | | | Light chain | | |
|---|---|---|---|---|---|---|
| Antibody | CDR | Sequence | SEQ ID NO | CDR | Sequence | SEQ ID NO |
| Cetuximab | VH-CDR1 | NYGVH | 175 | VL-CDR1 | RASQSIGTNIH | 178 |
| | VH-CDR2 | VIWSGGNTDYNTPF | 176 | VL-CDR2 | YASESIS | 179 |
| | | TS | | | | |
| | VH-CDR3 | ALTYYDYEFAY | 177 | VL-CDR3 | QQNNNWPTT | 180 |
| Panitumumab | VH-CDR1 | DYYWT | 181 | VL-CDR1 | QASQDISNYLN | 184 |
| | VH-CDR2 | HIYYSGNTNYNPSL | 182 | VL-CDR2 | DASNLET | 185 |
| | | KS | | | | |
| | VH-CDR3 | DRVTGAFDI | 183 | VL-CDR3 | QHFDHLPLA | 186 |
| Necitumumab | VH-CDR1 | DYYWS | 187 | VL-CDR1 | RASQSVSSYLA | 190 |
| | VH-CDR2 | YIYYSGSTDYNPSL | 188 | VL-CDR2 | DASNRAT | 191 |
| | | KS | | | | |
| | VH-CDR3 | VSIFGVGTFDY | 189 | VL-CDR3 | HQYGSTPLT | 192 |
| Imgatuzumab | VH-CDR1 | DYKIH | 193 | VL-CDR1 | RASQGINNYLN | 196 |
| | VH-CDR2 | YFNPNSGYSTYAQK | 194 | VL-CDR2 | NTNNLQT | 197 |
| | | FQG | | | | |
| | VH-CDR3 | LSPGGYYVMDA | 195 | VL-CDR3 | LQHNSFPT | 198 |
| Depatuxizumab | VH-CDR1 | DFAWN | 199 | VL-CDR1 | HSSQDINSNIG | 202 |
| | VH-CDR2 | YISYSGNTRYQPSL | 200 | VL-CDR2 | HGTNLDD | 203 |
| | | KS | | | | |
| | VH-CDR3 | AGRGFPY | 201 | VL-CDR3 | VQYAQFPWT | 204 |
| Losatuxizumab | VH-CDR1 | DFAWN | 199 | VL-CDR1 | HSSQDINSNIG | 202 |
| | VH-CDR2 | YISYNGNTRYQPSL | 205 | VL-CDR2 | HGTNLDD | 203 |
| | | KS | | | | |
| | VH-CDR3 | ASRGFPY | 206 | VL-CDR3 | VQYAQFPWT | 204 |
| Etevritamab | VH-CDR1 | NYGMH | 207 | VL-CDR1 | RSSQSLVHSDG | 210 |
| | | | | | NTYLS | |
| | VH-CDR2 | VIWYDGSDKYYAD | 208 | VL-CDR2 | RISRRFS | 211 |
| | | SVRG | | | | |
| | VH-CDR3 | DGYDILTGNPRDFD | 209 | VL-CDR3 | MQSTHVPRT | 212 |
| | | Y | | | | |
| AMG-595 | VH-CDR1 | SYGMH | 213 | VL-CDR1 | RSSQSL VHSDG | 210 |
| | | | | | NTYLS | |
| | VH-CDR2 | VIWYDGSNKYYVD | 214 | VL-CDR2 | KISNRFS | 216 |
| | | SVKG | | | | |
| | VH-CDR3 | DGWQQLAPFDY | 215 | VL-CDR3 | MQATQLPRT | 217 |
| Atezolizumab | VH-CDR1 | DSWIH | 218 | VL-CDR1 | RASQDVSTAVA | 221 |
| | VH-CDR2 | WISPYGGSTYYADS | 219 | VL-CDR2 | SASFLYS | 222 |
| | | VKG | | | | |
| | VH-CDR3 | RHWPGGFDY | 220 | VL-CDR3 | QQYLYHPAT | 223 |
| Avelumab | VH-CDR1 | SSYIM | 224 | VL-CDR1 | TGTSSDVGGYN | 227 |
| | | | | | YVS | |
| | VH-CDR2 | SIYPSGGITFYADTV | 225 | VL-CDR2 | DVSNRPS | 228 |
| | | KG | | | | |
| | VH-CDR3 | IKLGTVTTVDY | 226 | VL-CDR3 | SSYTSSSTRV | 229 |
| Durvalumab | VH-CDR1 | RYWMS | 230 | VL-CDR1 | RASQRVSSSYL | 233 |
| | | | | | A | |
| | VH-CDR2 | NIKQDGSEKYYVDS | 231 | VL-CDR2 | DASSRAT | 234 |
| | | VKG | | | | |
| | VH-CDR3 | EGGWFGELAFDY | 232 | VL-CDR3 | QQYGSLPWT | 235 |
| Trastuzumab | VH-CDR1 | DTYIH | 21 | VL-CDR1 | RASQDVNTAV | 24 |
| | | | | | A | |
| | VH-CDR2 | RIYPTNGYTRYADS | 22 | VL-CDR2 | SASFLYS | 25 |
| | | VKG | | | | |
| | VH-CDR3 | WGGDGFYAMDY | 23 | VL-CDR3 | QQHYTTPPT | 26 |
| Pertuzumab | VH-CDR1 | DYTMD | 33 | VL-CDR1 | KASQDVSIGVA | 36 |
| | VH-CDR2 | DVNPNSGGSIYNQR | 34 | VL-CDR2 | SASYRYT | 37 |
| | | FKG | | | | |
| | VH-CDR3 | NLGPSFYFDYW | 35 | VL-CDR3 | QQYYIYPYT | 38 |
| Onartuzumab | VH-CDR1 | SYWLH | 236 | VL-CDR1 | KSSQSLLYTSS | 239 |
| | | | | | QKNYLA | |
| | VH-CDR2 | MIDPSNSDTRFNPN | 237 | VL-CDR2 | WASTRES | 240 |
| | | FKD | | | | |
| | VH-CDR3 | YRSYVTPLDY | 238 | VL-CDR3 | QQYYAYPWT | 241 |
| Emibetuzumab | VH-CDR1 | DYYMH | 242 | VL-CDR1 | SVSSSVSSIYLH | 245 |
| | VH-CDR2 | RVNPNRRGTTYNQ | 243 | VL-CDR2 | STSNLAS | 246 |
| | | KFEG | | | | |
| | VH-CDR3 | ANWLDY | 244 | VL-CDR3 | QVYSGYPLT | 247 |
| Telisotuzumab | VH-CDR1 | AYTMH | 248 | VL-CDR1 | KSSESVDSYAN | 251 |
| | | | | | SFLH | |
| | VH-CDR2 | WIKPNNGLANYAQ | 249 | VL-CDR2 | RASTRES | 252 |
| | | KFQG | | | | |
| | VH-CDR3 | SEITTEFDY | 250 | VL-CDR3 | QQSKEDPLT | 253 |
| Datopotamab | VH-CDR1 | TAGMQ | 254 | VL-CDR1 | KASQDVSTAV | 257 |
| | | | | | A | |
| | VH-CDR2 | WINTHSGVPKYAE | 255 | VL-CDR2 | SASYRYT | 258 |
| | | DFKG | | | | |
| | VH-CDR3 | SGFGSSYWYFDV | 256 | VL-CDR3 | QQHYITPLT | 259 |
| Sacituzumab | VH-CDR1 | NYGMN | 260 | VL-CDR1 | KASQDVSIAVA | 263 |
| | VH-CDR2 | WINTYTGEPTYTDD | 261 | VL-CDR2 | SASYRYT | 264 |
| | | FKG | | | | |
| | VH-CDR3 | GGFGSSYWYFDV | 262 | VL-CDR3 | QQHYITPLT | 265 |
| Rovalpituzumab | VH-CDR1 | NYGMN | 266 | VL-CDR1 | KASQSVSNDVV | 269 |
| | VH-CDR2 | WINTYTGEPTYADD | 267 | VL-CDR2 | YASNRYT | 270 |
| | | FKG | | | | |
| | VH-CDR3 | IGDSSPSDY | 268 | VL-CDR3 | QQDYTSPWT | 271 |
| Tarlatamab | VH-CDR1 | SYYWS | 272 | VL-CDR1 | RASQRVNNNY | 275 |
| | | | | | LA | |
| | VH-CDR2 | YVYYSGTTNYNPSL | 273 | VL-CDR2 | GASSRAT | 276 |
| | | KS | | | | |
| | VH-CDR3 | IAVTGFYFDY | 274 | VL-CDR3 | QQYDRSPLT | 277 |
| Belantamab | VH-CDR1 | NYWMH | 278 | VL-CDR1 | SASQDISNYLN | 281 |
| | VH-CDR2 | ATYRGHSDTYYNQ | 279 | VL-CDR2 | YTSNLHS | 282 |
| | | KFKG | | | | |
| | VH-CDR3 | GAIYDGYDVLDN | 280 | VL-CDR3 | QQYRKLPWT | 283 |
| Ladiratuzumab | VH-CDR1 | DYYMH | 284 | VL-CDR1 | RSSQSLLHSSG | 287 |
| | | | | | NTYLE | |
| | VH-CDR2 | WIDPENGDTEYGPK | 285 | VL-CDR2 | KISTRFS | 288 |
| | | FQG | | | | |
| | VH-CDR3 | HNAHYGTWFAY | 286 | VL-CDR3 | FQGSHVPYT | 289 |
| Codrituzumab | VH-CDR1 | DYEMH | 99 | VL-CDR1 | RSSQSLVHSNR | 102 |
| | | | | | NTYLH | |
| | VH-CDR2 | ALDPKTGDTAYSQ | 100 | VL-CDR2 | KVSNRFS | 103 |
| | | KFKG | | | | |
| | VH-CDR3 | FYSYTY | 101 | VL-CDR3 | SQNTHVPPT | 104 |
| Aprutumab | VH-CDR1 | SYAMS | 290 | VL-CDR1 | SGSSSNIGNNY | 293 |
| | | | | | VS | |
| | VH-CDR2 | AISGSGTSTYYADS | 291 | VL-CDR2 | ENYNRPA | 294 |
| | | VKG | | | | |
| | VH-CDR3 | VRYNWNHGDWFD | 292 | VL-CDR3 | SSWDDSLNYW | 295 |
| | | P | | | V | |
| Bemarituzumab | VH-CDR1 | TYNVH | 296 | VL-CDR1 | KASQGVSNDV | 299 |
| | | | | | A | |
| | VH-CDR2 | SIYPDNGDTSYNQN | 297 | VL-CDR2 | SASYRYT | 300 |
| | | FKG | | | | |
| | VH-CDR3 | GDFAY | 298 | VL-CDR3 | QQHSTTPYT | 301 |
| Vofatamab | VH-CDR1 | STGIS | 302 | VL-CDR1 | RASQDVDTSLA | 305 |
| | VH-CDR2 | RIYPTSGSTNYADS | 303 | VL-CDR2 | SASFLYS | 306 |
| | | VKG | | | | |
| | VH-CDR3 | TYGIYDLYVDYTEY | 304 | VL-CDR3 | QQSTGHPQT | 307 |
| | | VMDY | | | | |
| Ramucirumab | VH-CDR1 | SYSMN | 308 | VL-CDR1 | RASQGIDNWLG | 311 |
| | VH-CDR2 | SISSSSSYIYYADSV | 309 | VL-CDR2 | DASNLDT | 312 |
| | | KG | | | | |
| | VH-CDR3 | VTDAFDI | 310 | VL-CDR3 | QQAKAFPPT | 313 |
| Rituximab | VH-CDR1 | SYNMH | 314 | VL-CDR1 | RSSKSLLHSNGI | 317 |
| | | | | | TYLY | |
| | VH-CDR2 | AIYPGNGDTSYNQK | 315 | VL-CDR2 | QMSNLVS | 318 |
| | | FKG | | | | |
| | VH-CDR3 | STYYGGDWYFNV | 316 | VL-CDR3 | AQNLELPYT | 319 |
| Obinutuzumab | VH-CDR1 | YSWIN | 320 | VL-CDR1 | RASQSVSSYLA | 323 |
| | VH-CDR2 | RIFPGDGDTDYNGK | 321 | VL-CDR2 | DASNRAT | 324 |
| | | FKG | | | | |
| | VH-CDR3 | NVFDGYWLVY | 322 | VL-CDR3 | QQRSNWPPT | 325 |
| Daratumumab | VH-CDR1 | SFAMS | 326 | VL-CDR1 | RASQSVSSYLA | 329 |
| | VH-CDR2 | AISGSGGGTYYADS | 327 | VL-CDR2 | DASNRAT | 330 |
| | | VKG | | | | |
| | VH-CDR3 | DKILWFGEPVFDY | 328 | VL-CDR3 | QQRSNWPPT | 331 |
| 1C1 | VH-CDR1 | HYMMA | 157 | VL-CDR1 | RASQSISTWLA | 160 |
| (Clone name) | VH-CDR2 | RIGPSGGPTHYADS | 158 | VL-CDR2 | KASNLHT | 161 |
| | | VKG | | | | |
| | VH-CDR3 | YDSGYDYVAVAGP | 159 | VL-CDR3 | QQYNSYSRT | 162 |
| | | AEYFQH | | | | |

### Antigen

As antigens to which the antigen-binding site described herein may specifically bind, the following non-limiting substances may be exemplified.

“Epidermal growth factor receptor (EGFR)”: It is a cell membrane receptor that regulates cell growth, division, survival, and death. In various cancers, the expression of EGFR is increased in tumor tissues. It is known that tumor tissues with the increased EGFR are invasive, metastatic, and highly resistant to anticancer agents. In one embodiment, the substance that inhibits EGFR may be cetuximab, panitumumab, necitumumab, imgatuzumab, depatuxizumab, or losatuxizumab, but is not limited thereto.

“Epidermal growth factor receptor variant 3 (EGFRvIII)”: It is a mutation in which exons 2 to 7 of EGFR are deleted. It is mainly reported in glioblastoma multiforme, and most patients with EGFRvIII-positive mutation have a poor prognosis. In one embodiment, the substance that inhibits EGFRvIII may be cetuximab, panitumumab, necitumumab, imgatuzumab, depatuxizumab, losatuxizumab, etevritamab, or AMG-595, but is not limited thereto.

Programmed death-ligand 1 (PD-L1)”: It is a protein overexpressed on the surface of cancer cells. It is a major cancer-specific antigen that plays an important role in inducing exhaustion and apoptosis of T cells and acquiring immune tolerance in cancer cells. In one embodiment, PD-L1 targeting anticancer antibody may be atezolizumab, avelumab, and durvalumab, but is not limited thereto.

“HER-2/neu (human epidermal growth factor receptor 2): It regulates cell proliferation through activation of PI3K/AkT. It is known that it is overexpressed in metastatic breast cancer and ovarian cancer, etc., and induces resistance against anticancer agents. The HER2/neu targeting anticancer agent may be trastuzumab or pertuzumab, but is not limited thereto.

“c-Met (mesenchymal-epithelial transition factor)”: It is a hepatocyte growth factor receptor. Its amplification or mutation is frequently reported in cancer cells, and it is known to promote tumor growth, metastasis, and malignancy. Specifically, the inhibitor of the protein may be onartuzumab, emibetuzumab, or telisotuzumab, but is not limited thereto.

“Trop-2 (tumor-associated calcium signal transducer 2)”: It is a cellular glycoprotein related to cancer cell growth and proliferation. It is known to be specifically overexpressed in non-small cell lung cancer and breast cancer. Specifically, the antibody targeting the protein may be datopotamab or sacituzumab, but is not limited thereto.

“DLL3 (delta-like ligand 3)”: It is a major cancer target antigen known to be expressed in approximately 85% of small cell lung cancer patients. Specifically, the antibody targeting the protein may be rovalpituzumab or tarlatamab, but is not limited thereto.

“BCMA (B-cell maturation antigen)”: It is an important factor in the survival and proliferation of myeloma cells and is a clinically proven target for treatment of multiple myeloma. Specifically, the antibody targeting the protein may be belantamab, but is not limited thereto.

“LIV-1 (zinc transporter ZIP6)”: It is a highly cancer-specific antigen overexpressed in breast cancer. Specifically, the antibody targeting the protein may be ladiratuzumab, but is not limited thereto.

“GPC-3 (glypican-3)”: It is a highly cancer-specific antigen that is specifically overexpressed in liver cancer. Specifically, the antibody targeting the protein may be codrituzumab, but is not limited thereto.

“FGFR (fibroblast growth factor receptor)”: It is a receptor for fibroblast growth factor (FGF), which regulates various biological processes including cell growth, differentiation, and migration. The FGFR gene is easily mutated, and these variants are commonly observed in breast cancer, uterine cancer, ovarian cancer, cervical cancer, and the like. The four FGFR genes are made of seven signaling receptors, of which FGFR2 and FGFR3 are highly cancer-specific antigens. The antibody targeting FGFR2 or FGFR3 may be aprutumab, bemarituzumab, or vofatamab, but is not limited thereto.

“Vascular endothelial growth factor receptor (VEGFR)”: It is a cell membrane receptor for a vascular endothelial growth factor that induces angiogenesis. The VEGFR inhibitor inhibits tumor growth and metastasis by inhibiting angiogenesis. In one embodiment, the VEGFR inhibitor may be ramucirumab, but is not limited thereto.

“CD20 (B lymphocyte antigen CD20)”: It is a protein expressed on the surface of B cells, which is used as a target protein for the treatment of B cell lymphoma. The CD20 inhibitor may be rituximab or obinutuzumab, but is not limited thereto.

“CD38 (cluster of differentiation 38)”: It is a protein that acts as a signal transduction receptor in immune cells and regulates cell proliferation and death. The inhibitor targeting the protein may be daratumumab, but is not limited thereto.

“EphA2 (EPH receptor A2)”: It is overexpressed in cancer cells and has a significant impact on the growth and metastasis of cancer cells. The antibody targeting the protein may be 1C1, but is not limited thereto.

The antigen-binding site that specifically binds to the antigens exemplified above may include CDR sequences as exemplified below.

### EGFR:

1) VH region (SEQ ID NO: 334) comprising the amino acid sequences of SEQ ID NO: 175 (VH-CDR1), SEQ ID NO: 176 (VH-CDR2) and SEQ ID NO: 177 (VH-CDR3), and VL region (SEQ ID NO: 335) comprising the amino acid sequences of SEQ ID NO: 178 (VL-CDR1), SEQ ID NO: 179 (VL-CDR2) and SEQ ID NO: 180 (VL-CDR3);
2) VH region (SEQ ID NO: 336) comprising the amino acid sequences of SEQ ID NO: 181 (VH-CDR1), SEQ ID NO: 182 (VH-CDR2) and SEQ ID NO: 183 (VH-CDR3), and VL region (SEQ ID NO: 337) comprising the amino acid sequences of SEQ ID NO: 184 (VL-CDR1), SEQ ID NO: 185 (VL-CDR2) and SEQ ID NO: 186 (VL-CDR3);
3) VH region (SEQ ID NO: 338) comprising the amino acid sequences of SEQ ID NO: 187 (VH-CDR1), SEQ ID NO: 188 (VH-CDR2) and SEQ ID NO: 189 (VH-CDR3), and VL region (SEQ ID NO: 339) comprising the amino acid sequences of SEQ ID NO: 190 (VL-CDR1), SEQ ID NO: 191 (VL-CDR2) and SEQ ID NO: 192 (VL-CDR3);
4) VH region (SEQ ID NO: 340) comprising the amino acid sequences of SEQ ID NO: 193 (VH-CDR1), SEQ ID NO: 194 (VH-CDR2) and SEQ ID NO: 195 (VH-CDR3), and VL region (SEQ ID NO: 341) comprising the amino acid sequences of SEQ ID NO: 196 (VL-CDR1), SEQ ID NO: 197 (VL-CDR2) and SEQ ID NO: 198 (VL-CDR3);
5) VH region (SEQ ID NO: 342) comprising the amino acid sequences of SEQ ID NO: 199 (VH-CDR1), SEQ ID NO: 200 (VH-CDR2) and SEQ ID NO: 201 (VH-CDR3), and VL region (SEQ ID NO: 343) comprising the amino acid sequences of SEQ ID NO: 202 (VL-CDR1), SEQ ID NO: 203 (VL-CDR2) and SEQ ID NO: 204 (VL-CDR3);
6) VH region (SEQ ID NO: 344) comprising the amino acid sequences of SEQ ID NO: 199 (VH-CDR1), SEQ ID NO: 205 (VH-CDR2) and SEQ ID NO: 206 (VH-CDR3), and VL region (SEQ ID NO: 345) comprising the amino acid sequences of SEQ ID NO: 202 (VL-CDR1), SEQ ID NO: 203 (VL-CDR2) and SEQ ID NO: 204 (VL-CDR3);

### EGFRvIII:

7) VH region (SEQ ID NO: 346) comprising the amino acid sequences of SEQ ID NO: 207 (VH-CDR1), SEQ ID NO: 208 (VH-CDR2) and SEQ ID NO: 209 (VH-CDR3), and VL region (SEQ ID NO: 347) comprising the amino acid sequences of SEQ ID NO: 210 (VL-CDR1), SEQ ID NO: 211 (VL-CDR2) and SEQ ID NO: 212 (VL-CDR3);
8) VH region (SEQ ID NO: 348) comprising the amino acid sequences of SEQ ID NO: 213 (VH-CDR1), SEQ ID NO: 214 (VH-CDR2) and SEQ ID NO: 215 (VH-CDR3), and VL region (SEQ ID NO: 349) comprising the amino acid sequences of SEQ ID NO: 210 (VL-CDR1), SEQ ID NO: 216 (VL-CDR2) and SEQ ID NO: 217 (VL-CDR3);

### PD-L1:

9) VH region (SEQ ID NO: 350) comprising the amino acid sequences of SEQ ID NO: 218 (VH-CDR1), SEQ ID NO: 219 (VH-CDR2) and SEQ ID NO: 220 (VH-CDR3), and VL region (SEQ ID NO: 351) comprising the amino acid sequences of SEQ ID NO: 221 (VL-CDR1), SEQ ID NO: 222 (VL-CDR2) and SEQ ID NO: 223 (VL-CDR3);
10) VH region (SEQ ID NO: 352) comprising the amino acid sequences of SEQ ID NO: 224 (VH-CDR1), SEQ ID NO: 225 (VH-CDR2) and SEQ ID NO: 226 (VH-CDR3), and VL region (SEQ ID NO: 353) comprising the amino acid sequences of SEQ ID NO: 227 (VL-CDR1), SEQ ID NO: 228 (VL-CDR2) and SEQ ID NO: 229 (VL-CDR3);
11) VH region (SEQ ID NO: 354) comprising the amino acid sequences of SEQ ID NO: 230 (VH-CDR1), SEQ ID NO: 231 (VH-CDR2) and SEQ ID NO: 232 (VH-CDR3), and VL region (SEQ ID NO: 355) comprising the amino acid sequences of SEQ ID NO: 233 (VL-CDR1), SEQ ID NO: 234 (VL-CDR2) and SEQ ID NO: 235 (VL-CDR3);

### HER2:

12) VH region (SEQ ID NO: 356) comprising the amino acid sequences of SEQ ID NO: 21 (VH-CDR1), SEQ ID NO: 22 (VH-CDR2) and SEQ ID NO: 23 (VH-CDR3), and VL region (SEQ ID NO: 357) comprising the amino acid sequences of SEQ ID NO: 24 (VL-CDR1), SEQ ID NO: 25 (VL-CDR2) and SEQ ID NO: 26 (VL-CDR3);
13) VH region (SEQ ID NO: 27) comprising the amino acid sequences of SEQ ID NO: 33 (VH-CDR1), SEQ ID NO: 34 (VH-CDR2) and SEQ ID NO: 35 (VH-CDR3), and VL region (SEQ ID NO: 28) comprising the amino acid sequences of SEQ ID NO: 36 (VL-CDR1), SEQ ID NO: 37 (VL-CDR2) and SEQ ID NO: 38 (VL-CDR3);
   c-Met:
14) VH region (SEQ ID NO: 358) comprising the amino acid sequences of SEQ ID NO: 236 (VH-CDR1), SEQ ID NO: 237 (VH-CDR2) and SEQ ID NO: 238 (VH-CDR3), and VL region (SEQ ID NO: 359) comprising the amino acid sequences of SEQ ID NO: 239 (VL-CDR1), SEQ ID NO: 240 (VL-CDR2) and SEQ ID NO: 241 (VL-CDR3);
15) VH region (SEQ ID NO: 360) comprising the amino acid sequences of SEQ ID NO: 242 (VH-CDR1), SEQ ID NO: 243 (VH-CDR2) and SEQ ID NO: 244 (VH-CDR3), and VL region (SEQ ID NO: 361) comprising the amino acid sequences of SEQ ID NO: 245 (VL-CDR1), SEQ ID NO: 246 (VL-CDR2) and SEQ ID NO: 247 (VL-CDR3);
16) VH region (SEQ ID NO: 362) comprising the amino acid sequences of SEQ ID NO: 248 (VH-CDR1), SEQ ID NO: 249 (VH-CDR2) and SEQ ID NO: 250 (VH-CDR3), and VL region (SEQ ID NO: 363) comprising the amino acid sequences of SEQ ID NO: 251 (VL-CDR1), SEQ ID NO: 252 (VL-CDR2) and SEQ ID NO: 253 (VL-CDR3);

### Trop-2:

17) VH region (SEQ ID NO: 364) comprising the amino acid sequences of SEQ ID NO: 254 (VH-CDR1), SEQ ID NO: 255 (VH-CDR2) and SEQ ID NO: 256 (VH-CDR3), and VL region (SEQ ID NO: 365) comprising the amino acid sequences of SEQ ID NO: 257 (VL-CDR1), SEQ ID NO: 258 (VL-CDR2) and SEQ ID NO: 259 (VL-CDR3);
18) VH region (SEQ ID NO: 366) comprising the amino acid sequences of SEQ ID NO: 260 (VH-CDR1), SEQ ID NO: 261 (VH-CDR2) and SEQ ID NO: 262 (VH-CDR3), and VL region (SEQ ID NO: 367) comprising the amino acid sequences of SEQ ID NO: 263 (VL-CDR1), SEQ ID NO: 264 (VL-CDR2) and SEQ ID NO: 265 (VL-CDR3);

### DLL3:

19) VH region (SEQ ID NO: 368) comprising the amino acid sequences of SEQ ID NO: 266 (VH-CDR1), SEQ ID NO: 267 (VH-CDR2) and SEQ ID NO: 268 (VH-CDR3), and VL region (SEQ ID NO: 369) comprising the amino acid sequences of SEQ ID NO: 269 (VL-CDR1), SEQ ID NO: 270 (VL-CDR2) and SEQ ID NO: 271 (VL-CDR3);
20) VH region (SEQ ID NO: 370) comprising the amino acid sequences of SEQ ID NO: 272 (VH-CDR1), SEQ ID NO: 273 (VH-CDR2) and SEQ ID NO: 274 (VH-CDR3), and VL region (SEQ ID NO: 371) comprising the amino acid sequences of SEQ ID NO: 275 (VL-CDR1), SEQ ID NO: 276 (VL-CDR2) and SEQ ID NO: 277 (VL-CDR3);

### BCMA:

21) VH region (SEQ ID NO: 372) comprising the amino acid sequences of SEQ ID NO: 278 (VH-CDR1), SEQ ID NO: 279 (VH-CDR2) and SEQ ID NO: 280 (VH-CDR3), and VL region (SEQ ID NO: 373) comprising the amino acid sequences of SEQ ID NO: 281 (VL-CDR1), SEQ ID NO: 282 (VL-CDR2) and SEQ ID NO: 283 (VL-CDR3);

### LIV-1:

22) VH region (SEQ ID NO: 374) comprising the amino acid sequences of SEQ ID NO: 284 (VH-CDR1), SEQ ID NO: 285 (VH-CDR2) and SEQ ID NO: 286 (VH-CDR3), and VL region (SEQ ID NO: 375) comprising the amino acid sequences of SEQ ID NO: 287 (VL-CDR1), SEQ ID NO: 288 (VL-CDR2) and SEQ ID NO: 289 (VL-CDR3);

### GPC-3:

23) VH region (SEQ ID NO: 87) comprising the amino acid sequences of SEQ ID NO: 99 (VH-CDR1), SEQ ID NO: 100 (VH-CDR2) and SEQ ID NO: 101 (VH-CDR3), and VL region (SEQ ID NO: 88) comprising the amino acid sequences of SEQ ID NO: 102 (VL-CDR1), SEQ ID NO: 103 (VL-CDR2) and SEQ ID NO: 104 (VL-CDR3);

### FGFR2:

24) VH region (SEQ ID NO: 376) comprising the amino acid sequences of SEQ ID NO: 290 (VH-CDR1), SEQ ID NO: 291 (VH-CDR2) and SEQ ID NO: 292 (VH-CDR3), and VL region (SEQ ID NO: 377) comprising the amino acid sequences of SEQ ID NO: 293 (VL-CDR1), SEQ ID NO: 294 (VL-CDR2) and SEQ ID NO: 295 (VL-CDR3);
25) VH region (SEQ ID NO: 378) comprising the amino acid sequences of SEQ ID NO: 296 (VH-CDR1), SEQ ID NO: 297 (VH-CDR2) and SEQ ID NO: 298 (VH-CDR3), and VL region (SEQ ID NO: 379) comprising the amino acid sequences of SEQ ID NO: 299 (VL-CDR1), SEQ ID NO: 300 (VL-CDR2) and SEQ ID NO: 301 (VL-CDR3);

### FGFR3:

26) VH region (SEQ ID NO: 380) comprising the amino acid sequences of SEQ ID NO: 302 (VH-CDR1), SEQ ID NO: 303 (VH-CDR2) and SEQ ID NO: 304 (VH-CDR3), and VL region (SEQ ID NO: 381) comprising the amino acid sequences of SEQ ID NO: 305 (VL-CDR1), SEQ ID NO: 306 (VL-CDR2) and SEQ ID NO: 307 (VL-CDR3);

### VEGFR2:

27) VH region (SEQ ID NO: 382) comprising the amino acid sequences of SEQ ID NO: 308 (VH-CDR1), SEQ ID NO: 309 (VH-CDR2) and SEQ ID NO: 310 (VH-CDR3), and VL region (SEQ ID NO: 383) comprising the amino acid sequences of SEQ ID NO: 311 (VL-CDR1), SEQ ID NO: 312 (VL-CDR2) and SEQ ID NO: 313 (VL-CDR3);

### CD20:

28) VH region (SEQ ID NO: 384) comprising the amino acid sequences of SEQ ID NO: 314 (VH-CDR1), SEQ ID NO: 315 (VH-CDR2) and SEQ ID NO: 316 (VH-CDR3), and VL region (SEQ ID NO: 385) comprising the amino acid sequences of SEQ ID NO: 317 (VL-CDR1), SEQ ID NO: 318 (VL-CDR2) and SEQ ID NO: 319 (VL-CDR3);
29) VH region (SEQ ID NO: 386) comprising the amino acid sequences of SEQ ID NO: 320 (VH-CDR1), SEQ ID NO: 321 (VH-CDR2) and SEQ ID NO: 322 (VH-CDR3), and VL region (SEQ ID NO: 387) comprising the amino acid sequences of SEQ ID NO: 323 (VL-CDR1), SEQ ID NO: 324 (VL-CDR2) and SEQ ID NO: 325 (VL-CDR3);

### CD38:

30) VH region (SEQ ID NO: 388) comprising the amino acid sequences of SEQ ID NO: 326 (VH-CDR1), SEQ ID NO: 327 (VH-CDR2) and SEQ ID NO: 328 (VH-CDR3), and VL region (SEQ ID NO: 389) comprising the amino acid sequences of SEQ ID NO: 329 (VL-CDR1), SEQ ID NO: 330 (VL-CDR2) and SEQ ID NO: 331 (VL-CDR3);

### EphA2:

31) VH region (SEQ ID NO: 143) comprising the amino acid sequences of SEQ ID NO: 157 (VH-CDR1), SEQ ID NO: 158 (VH-CDR2) and SEQ ID NO: 159 (VH-CDR3), and VL region (SEQ ID NO: 145) comprising the amino acid sequences of SEQ ID NO: 160 (VL-CDR1), SEQ ID NO: 161 (VL-CDR2) and SEQ ID NO: 162 (VL-CDR3).

Table 2 below shows the nucleotide sequence and the polypeptide sequence of an exemplary signal sequence for efficient expression of the fusion proteins according to various embodiments. When the above antibodies are expressed in mammalian cells, SEQ ID NO: 333 may be used as the signal sequence, but is not limited thereto.

TABLE 2

| Name | Sequence | SEQ ID NO |
|---|---|---|
| Nucleotide | ATGACACGCCTCACAGTTCTTG | 332 |
| sequence | CCTTGCTGGCTGGACTTCTTGC | |
| | CTCATCCAGGGCA | |
| Polypeptide | MTRLTVLALLAGLLASSRA | 333 |
| sequence | | |

Table 3 below shows the variable region polypeptide sequences of anticancer antibodies, which are described as antigen-binding sites of various fusion proteins described herein. The fusion proteins according to exemplary embodiments may comprise or consist of these variable region polypeptides.

TABLE 3

| | | | | SEQ |
|---|---|---|---|---|
| Antigen | Antibody | Variable | | ID |
| | | Region | Polypeptide sequence of variable region | NO |
| EGFR | Cetuximab | VH | QVQLKQSGPGLVQPSQSLSITCTVSGFSLTNYGVHWVRQSPGKGLEWLGVI | 334 |
| | | | WSGGNTDYNTPFTSRLSINKDNSKSQVFFKMNSLQSNDTAIYYCARALTYY | |
| | | | DYEFAYWGQGTLVTVSA | |
| | | VL | DILLTQSPVILSVSPGERVSFSCRASQSIGTNIHWYQQRTNGSPRLLIKYA | 335 |
| | | | SESISGIPSRFSGSGSGTDFTLSINSVESEDIADYYCQQNNNWPTTFGAGT | |
| | | | KLELK | |
| | Panitumumab | VH | QVQLQESGPGLVKPSETLSLTCTVSGGSVSSGDYYWTWIRQSPGKGLEWIG | 336 |
| | | | HIYYSGNTNYNPSLKSRLTISIDTSKTQFSLKLSSVTAADTAIYYCVRDRV | |
| | | | TGAFDIWGQGTMVTVSS | |
| | | VL | DIQMTQSPSSLSASVGDRVTITCQASQDISNYLNWYQQKPGKAPkLLIYDA | 337 |
| | | | SNLETGVPSRFSGSGSGTDFTFTISSLQPEDIATYFCQHFDHLPLAFGGGT | |
| | | | KVEIK | |
| | Necitumumab | VH | QVQLQESGPGLVKPSQTLSLTCTVSGGSISSGDYYWSWIRQPPGKGLEWIG | 338 |
| | | | YIYYSGSTDYNPSLKSRVTMSVDTSKNQFSLKVNSVTAADTAVYYCARVSI | |
| | | | FGVGIFDYWGQGTLVTVSS | |
| | | VL | EIVMTQSPATLSLSPGERATLSCRASQSVSSYLAWYQQKPGQAPELLIYDA | 339 |
| | | | SNRATGIPARFSGSGSGTDFTLTISSLEPEDFAVYYCHQYGSTPLIFGGGT | |
| | | | KAEIK | |
| | Imgatuzumab | VH | QVQLVQSGAEVKKPGSSVKVSCKASGFTFTDYKIHWVRQAPGQGLEWNGYF | 340 |
| | | | NPNSGYSTYAQKFQGRYTITADKSTSTAYMELSSLRSEDTAVYYCARLSPG | |
| | | | GYYVNDAWGQGTTYTYSS | |
| | | VL | DIQMTQSPSSLSASYGDRYTITCRASQGINNYLNWYQQKPGKAPERLIYNT | 341 |
| | | | NNLQTGVPSRFSGSGSGTEFTLTISSLQPEDFATYYCLQHNSFPTFGQGTK | |
| | | | LEIK | |
| | Depatuxizumab | VH | QVQLQESGPGLVKPSQTLSLTCTVSGYSISSDFAWNWIRQPPGKGLEWMGY | 342 |
| | | | ISYSGNTRYQPSLKSRITISRDTSKNQFFLKLNSVIAADTATYYCVTAGRG | |
| | | | FPYWGQGTLVTVSS | |
| | | VL | DIQMTQSPSSMSVSVGDRVTITCHSSQDINSNIGWLQQKTGKSFKGLIYHG | 343 |
| | | | INLDDGVPSRFSGSGSGTDYTLTISSLQPEDFATYYCVQYAQFPWTFGGGT | |
| | | | ELEIK | |
| | Losatuxiszumab | VH | EVQLQESGPGLVKPSQTLSLTCTYSGYSISRDFAWNWIRQPPGKGLEWMGY | 344 |
| | | | ISYNGNTRYQPSLESRITISRDTSKNQFFLELNSVTAADTATYYCVTASRG | |
| | | | FPYWGQGTLVTVSS | |
| | | VL | DIQMTQSPSSMSVSVGDRVTITCHSSQDINSNIGWLQQKPGKSFKGLIYHG | 345 |
| | | | TNLDDGVPSRFSGSGSGTDYTLTISSLQPEDFATYYCVQYAQFPWTFGGGT | |
| | | | KLEIK | |
| EGFRv | Etevritamab | VH | QVQLVESGGGNVQSGRSLRLSCAASGFTFRHYGNHIVRQADGKCLEWYAVI | 346 |
| III | | | WYDGSDKYYADSYRGRFTISRDNSENTLYLQMNSLRAEDTAVYYCARDGYD | |
| | | | ILTGNPRDEDYWGQGILVTVSS | |
| | | VL | DTVMTQTPLSSHVTLGQPASISCRSSQSLVHSDGNTYLSWLQQRPGQPPRL | 347 |
| | | | LIYRISRRFSGVPDRFSGSGAGTDFTLEISRVEAEDVGVYYCMQSTHVPRT | |
| | | | FGCGTKVEIK | |
| | ANG-505 | VH | QVQLVESGGGVVQFGRSLRLSCAASGFTFSSYGMHNVRQAPGEGLEWVAVI | 348 |
| | | | WYDGSNEYYVDSVEGRFTISNQNSKNTLYLQMNSLRAEDTAVYYCARDGWQ | |
| | | | QLAPFDYWGQGTLVTVSS | |
| | | VL | DIVMTQTTLSSPVTLGQPASISCRSSQSLVHSDGNTYLSWLHQRFGQPPRL | 349 |
| | | | LIYKISNRFSGVPDRFSGSGAGTAFTLEISKVEAEDVGVYYCMQATQLPRI | |
| | | | FGQGIKVEIK | |
| PD-L1 | Atezolizumab | VH | EVQLVESGGGLVQPGGSLRLSCAASGFTFSDSWIHNVRQANGKGLEWYAWI | 350 |
| | | | SPYGGSTYYADSVEGRFTISADTSKNTAYLQMNSLRAEDTAVYYCARRHWP | |
| | | | GGFDYWGQGILVIVSS | |
| | | VL | DIQMTQSPSSLSASNGDKVTITCRASQDVSTAVAWYQQEFGKAPKLLIYSA | 351 |
| | | | SFLYSGVTSRESGSGSGIDFTLTISSLQPEDFATYYQQQYLYHPATFGQGT | |
| | | | EVEIK | |
| | Avelumab | VH | EVQLLESGGGLVQPGGSLRLSCAASGFTFSSYIMMWVRQAPGKGLEWVSSI | 352 |
| | | | YTSGGITFYADTVKGRFTISKDNSKNTLYLQNNSLRAEDTAVYYCARIKLG | |
| | | | TVTTVDYWGQGTLVTVSS | |
| | | VL | QSALTQPASVSGSPGQSITISCTGTSSDVGGYNYVSWYQQHPGKAPELMIY | 353 |
| | | | DVSNRPSGVSNRFSGSKSGNTASLTISGLQAEDEADYYCSSYTSSSTRVFG | |
| | | | TGTKYTYL | |
| | Durvalumab | VH | EVQLVESGGGLYQDGGSLRLSCAASGFTFSRYWMSNVRQAPGKGLEWVANT | 354 |
| | | | KQDGSEKYYVDSVEGRFTISRDNAKNSLYLQMNSLRAEDTAVYYCAREGGW | |
| | | | FGELAFDYWGQGILYTVSS | |
| | | VL | EIVLTQSPGTLSLSPGERATLSCRASQRYSSSYLAWYQQKPGQAPRLLTYD | 355 |
| | | | ASSRATGIPDRFSGSGSGTDFTLTISRLEPEDFAVYYCQQYGSLFWTFGQG | |
| | | | TKVEIK | |
| HER2 | Trastuzumab | VH | EVQLVESGGGLVQNGGSLRISCAASGFNIKDTYIHWVRQAPGKGLENVARI | 356 |
| | | | YTTNGYTEYADSVEGRFTISADTSKNTAYLQMNSLRAEDTAVYYCSRWGGD | |
| | | | GFYAMDYWGQGTLVTVSS | |
| | | VL | DIQMTQSPSSLSASVGDRVTITCRASQDVNTAVAWYQQKPGKAPELLIYSA | 357 |
| | | | SFLYSGVPSRFSGSRSGIDFTLTISSLQPEDFATYYCQCHYTITPTFGQGT | |
| | | | KVEIK | |
| | Pertuzumab | VH | EVQLVESGGGLVQPGGSLRLSCAASGFIFTDYINDNVRQAFGKGLEWVADY | 27 |
| | | | NPNSGGSIYNQRFKGRFILSVDRSKNILYLQINSLRAEDTAVYYCARNEGP | |
| | | | SFYFDYWGQGTLVTVSS | |
| | | VL | DIQMTQSPSSLSASVGDRVTITCKASQDVSIGVAWYQQKPGRAPKLLIYSA | 28 |
| | | | SYRYTGVPSRFSGSGSGIDFILTISSLQPEDFATYYCQQYYTYPYTFGQGT | |
| | | | KVEIR | |
| c-Net | Onartuzumab | VH | EVQLVESGGGLVQPGGSLELSCAASGYTFTSYWLHWVRQAPGKGLETVGNI | 358 |
| | | | DFSNSDTRFNPNFKDRFTISADTSKNTAYLQMNSLRAEDTAVYYCATYRSY | |
| | | | VIPLDYWGQGTLVTVSS | |
| | | VL | DIQMTQSPSSLSASVGDRYTITCKSSQSLLYTSSQKNYLAWYQQKPGKAPK | 359 |
| | | | LLIYWASTRESGVFSRFSGSGSGTDFTLTISSLQPEDFATYYCQQYYAYPW | |
| | | | TFGQGTKVEIK | |
| | Emibetuzumab | VH | QVQLVQSGAEVEKPGASVKVSCKASGYTFTDYYMHWVRQAPGQGLEWMGRV | 360 |
| | | | NPNRRGTTYNQKFEGRVTMTTDTSTAYMELRSLRSDDTAVYYCARANWL | |
| | | | DYWGQGTTVTVSS | |
| | | VL | DIQMTQSPSSLSASVGDRYTITCSVSSSVSSTYLHTYQQKPGKAPELLIYS | 361 |
| | | | TSNLASGVPSRFSGSGSGTDFILTISSLQPEDFATYYCQVYSGYPLTFGGG | |
| | | | TEVEIK | |
| | Telisotuzumab | VH | QVQLVQSGAEVEKPGASVKYSCKASGYIFTAYIMHWVRQAFGQGLEWMGWI | 362 |
| | | | KPNNGLANYAQKFQGRYTMIRDTSISTAYMELSRLRSDDTAVYYCARSEIT | |
| | | | TEFDYWGQGILVTVSS | |
| | | VL | DIVWTQSPDSLAVSLGERATINCKSSESVDSYANSRLHWYQQKPGQPPKLL | 363 |
| | | | IYRASTRESGVPDRFSGSGSGTDFTLTISSLQAEDVAVYYCQQSKEDPLTF | |
| | | | GGGTKVEIK | |
| TROP-2 | Datopotamab | VH | QVQLVQSGAEVKKPGASVKVSCKASGYTFITAGMQWVRQAPGQGLEWMGWI | 364 |
| | | | NTHSGVPKYAEDFKGRVTISADTSTSTAYLQLSSLKSEDTAVYYCARSGFG | |
| | | | SSYWYFDVWGQGTLVTVSS | |
| | | VL | DIQMTQSPSSLSASVGDRVTITCKASQDVSTAVAWYQQKPGKAPKLLIYSA | 365 |
| | | | SYRYTGVPSRFSGSGSGTDFTLTISSLQPEDFAVYYCQQHYITPLTFGQGT | |
| | | | KLEIK | |
| | Sac [symbol] tuzumab | VH | QVQLQQSGSELKKPGASVKVSCKASGYTFTNYGMNWVKQAPGQGLKWMGWI | 366 |
| | | | NTYTGEPTYTDDFKGRFAFSLDTSVSTAYLQISSLKADDTAVYFCARGGFG | |
| | | | SSYWYFDVWGQGSLVTVSS | |
| | | | DIQLTQSPSSLSASVGDRVSITCKASQDVSIAVAWYQQKPGKAPKLLIYSA | 367 |
| | | | SYRYTGVPDRFSGSGTDFTLTISSLQPEDFAVYYCQQHYITPLTFGAGTKV | |
| | | | EIK | |
| DLL3 | Rovalpituzumab | VH | QVQLVQSGAEVKKPGASVKVSCKASGYTFTNYGMNWVRQAPGQGLEWMGWI | 368 |
| | | | NTYTGEPTYADDFKGRVTMTIDTSTSTAYMELRSLRSDDTAVYYCARIGDS | |
| | | | SPSDYWGQGTLVTVSS | |
| | | VL | EIVMTQSPATLSVSPGERAILSCKASQSVSNDVVWYQQRIGQAPRLLIYYA | 369 |
| | | | SNRYTGIPARFSGSGSGTEFTLTISSLQSEDFAVYYCQQDYTSPWTFGQGT | |
| | | | KLEIK | |
| | Tar [symbol] atamab | VH | QVQLQESGPGLVKPSETLSLTCTVSGGSISSYYWSWIRQPPGKCLEWIGYV | 370 |
| | | | YYSGTTNYNPSLKSRVTISVDISKNQFSLKLSSVIAADTAVYYCASIAVTG | |
| | | | FYFDYWGQGILVTVSS | |
| | | | EIVLTQSPGTLSLSPGERVILSCRASQRVNNNYLAWYQQRPGQAPRLLIYG | 371 |
| | | | ASSRATGIPDRFSGSGSGTDFTLTISRLEPEDFAVYYCQQYDRSELTFGCG | |
| | | | TKLEIK | |
| BCMA | Belantamab | VH | QVQLVQSGAEVEKPGSSVKVSCKASGGTFSVYWMHWYRQAPGQGLETMGAT | 372 |
| | | | YRGHSDTYYNQKFKGRVTITADESTSTAYMELSSLRSEDTAVYYCARGAIY | |
| | | | DGYDVLDNWGQGTLVTVSS | |
| | | | DIQMTQSPSSLSASVGDRVTITCSASQDISNYLNWYQQRPGKAPELLIYYT | 373 |
| | | | SNLHSGVPSRFSGSGSGIDFTLTISSLQPEDFATYYCQQYRKLFTIFGQGI | |
| | | | KLEIK | |
| LIV-1 | Ladiratuzumab | VH | QVQLVQSGAEVEKTGASVEYSCKASGLTIEDYYMHWVRQAPGQGLETMGWI | 374 |
| | | | DRENGDTEYGPKFQGRVTMTRQTSINTAYMELSRLESDDTAVYYCAVHNAH | |
| | | | YGTWFAYWGQGTLVTVSS | |
| | | | DVVMTQSPLSLPVTLGQPASISCRSSQSLLHSSGNTYLEWYQQRPGQSPRP | 375 |
| | | | LIYKISTRFSGVPDRFSGSGSGTDFILKISRVEAEDVGVYYCFQGSHYPYT | |
| | | | FGGGTKVEIK | |
| GPC-3 | Codrituzumab | VH | QVQLVQSGAEVKKPGASVKVSCKASGYTFTDYEMHWVRQAPGQGLEWMGAL | 87 |
| | | | DPKTGDTAYSQKFKGRVTLTADKSTSTAYMELSSLTSEDTAVYYCTRFYSY | |
| | | | TYWGQGTLVTVSS | |
| | | VL | DVVMTQSPLSLPVTPGEPASISCRSSQSLVHSNRNTYLHWYLQKPGQSPQL | 88 |
| | | | LIYKVSNRFSGVPDRFSGSGSGTDFTLKISRVEAEDVGVYYCSQNTHYPPT | |
| | | | FGQGTELEIK | |
| FGFR2 | Aprutumab | VH | EVQLLESGGGLVQPGGSLRLSCAASGFTFSSYAMSWVRQAPGKGLEWVSAI | 376 |
| | | | SGSGTSTYYADSVKGRATISRDNSKNTLYLQMNSLRAEDTAVYYCARVRYN | |
| | | | WNHGDWFDPWGQGTLVTVSS | |
| | | 11 | QSVLTQPPSASGTPGQRVTISCSGSSSNIGNNYVSWYQQLPGTAPELLIYE | 377 |
| | | | NYNRPAGVPDRFSGSKSGTSASLAISGLRSEDEADYYCSSWDDSLNYWVFG | |
| | | | GGTKLTVL | |
| | Bemaritusumab | VH | QVQLVQSGAEVKKPGSSVKVSCKASGYIFTTYNVHWVRQAPGQGLEWIGSI | 378 |
| | | | YPDNGDTSYNQNFKGRATITADKSTSTAYMELSSLRSEDTAVYYCARGDFA | |
| | | | YWGQGTLVTVSS | |
| | | | DIQMTQSPSSLSASVGDRVTITCKASQGYSNDVAWYQQKPGKAPKLLIYSA | 379 |
| | | | SYRYTGVPSRFSGSGSGIDFIFTISSLQPEDIATYYCQQHSTIPYTFGQGT | |
| | | | KLEIK | |
| FGER3 | Vofatamab | VH | EVQLVESGGGLVQPGGSLRLSCAASGFTFTSTGISWVRQAPGKGLEWYGRI | 380 |
| | | | YPTSGSTNYADSVKGRFTISADTSKNTAYLQMNSLRAEDTAVYYCARTYGI | |
| | | | YDLYVDYTEYVMDYWGQGTLVTVSS | |
| | | | DIQMTQSPSSLSASVGDRYTITCRASQDVDTSLAWYKQKPGKAPELLIYSA | 381 |
| | | | SFLYSGVPSRFSGSGSGIDFILTISSLQPEDFATYYCQQSTGHPQTFGQGT | |
| | | | KVEIK | |
| VEGFR 2 | Ramucirumab | VH | EVQLVQSGGGLVKPGGSLRLSCAASGFTFSSYSMNWVRQAPGKGLEWVSSI | 382 |
| | | | SSSSSYIYYADSVKGRFTISRDNAKNSLYLQMNSLRAEDTAVYYCARVTDA | |
| | | | FDIWGQGTMVTVSS | |
| | | VL | DIQMTQSPSSVSASIGDRVTITCRASQGIDNWLGWYQQKPGKAPKLLIYDA | 383 |
| | | | SNLDTGVPSRFSGSGSGTYFTLTISSLQAEDFAVYFCQQAKAFPPTFGGGT | |
| | | | KVDIK | |
| CD20 | Rituximab | VH | QVQLQQPGAELVKPGASVKMSCKASGYTFTSYNMHWVKQTPGRGLEWIGAI | 384 |
| | | | YPGNGDTSYNQKFKGKATLTADKSSSTAYMQLSSLTSEDSAVYYCARSTYY | |
| | | | GGDWYFNVWGAGTTVTVSA | |
| | | VL | QIVLSQSPAILSASPGEKVTMTCRASSSVSYIHWFQQKPGSSPKPWIYATS | 385 |
| | | | NLASGVPVRFSGSGSGTSYSLTISRVEAEDAATYYCQQWTSNPPTFGGGTK | |
| | | | LEIK | |
| | Obinutuzumab | VH | QVQLVQSGAEVKKPGSSVKVSCKASGYAFSYSWINWYRQAPGQGLEWNGRI | 386 |
| | | | FPGDGDTDYNGKFKGRVTITADKSTSTAYMELSSLRSEDTAVYYCARNVFD | |
| | | | GYWLVYWGQGTLVTVSS | |
| | | VL | DIVMTQTPLSLPVIPGETASISCRSSKSLLHSNGITYLYWYLQKPGQSPQL | 387 |
| | | | LIYQMSNLVSGVPDRFSGSGSGTDFTLKISRVEAEDVGVYYCAQNLELPYT | |
| | | | FGGGIKVEIK | |
| CDSS | Daratimumab | VH | EVQLLESGGGLVQPGGSLRLSCAVSGFTFNSFAMSWVRQAPGKGLEWVSAI | 388 |
| | | | SGSGGGTYYADSVKGRFTISRDNSKNTLYLQMNSLRAEDTAVYFCAKDKIL | |
| | | | WFGEPVFDYWGQGTLVTVSS | |
| | | VL | EIVLTQSPATLSLSPGERATLSCRASQSVSSYLAWYQQKPGQAPRLLIYDA | 389 |
| | | | SNRATGIPARFSGSGSGTDFTLTISSLEPEDFAVYYCQQRSNWPPTFGQGT | |
| | | | KVEIK | |
| Eph42 | 1C1 | VH | EVQLLESGGGLVQPGGSLRLSCAASGFTFSHYMMAWVRQAPGKGLEWVSRI | 143 |
| | | | GPSGGPTHYADSVKGRFTISRDNSKNTLYLQMNSLRAEDTAVYYCAGYDSG | |
| | | | YDYVAVAGPAEYFQHWGQGTLVTVSS | |
| | | VL | DIQMTQSPSSLSASVGDRVTITCRASQSISTWLAWYQQKPGKAPKLLTYKA | 145 |
| | | | SNLHTGVPSRFSGSGSGTEFSLTISGLQPDDFATYYCQQYNSYSRTFGQGT | |
| | | | KVEIK | |

[symbol]

indicates data missing or illegible when filed

Table 4 below shows the nucleotide sequences encoding variable regions of anticancer antibodies, which are described as antigen-binding sites of various fusion proteins described herein.

TABLE 4

| | | | | SEQ |
|---|---|---|---|---|
| | | Variable | | ID |
| Antigen | Antibody | Region | Nucleotide sequence of variable region | NO |
| EGFR | Cetuximab | VH | CAAGTGCAGCTTAAACAGAGTGGACCAGGGCTCGTTCAGCCTTCTCAAAGTTTG | 390 |
| | | | AGCATAACATGTACCGTAAGTGGTTTTTCACTGACTAACTACGGAGTGCATTGG | |
| | | | GTTCGACAGTCCCCTGGAAAGGGTCTCGAATGGCTTGGAGTTATTTGGAGTGGC | |
| | | | GGTAACACTGACTACAACACACCATTCACCTCTCGGCTGTCTATAAACAAGGAT | |
| | | | AACTCTAAGTCACAGGTCTTTTTCAAGATGAACAGCCTCCAAAGTAACGACACC | |
| | | | GCCATCTATTACTGTGCAAGAGCCTTGACCTACTACGATTACGAGTTTGCCTAT | |
| | | | TGGGGTCAGGGAACTCAAAGTCAGTGCT | |
| | | VL | GACATCCTTCTCACTCAGTCCCCCGTAATTCTGAGCGTGTCTCCCGGCGAGCGC | 301 |
| | | | GTCAGTTTCTCATGCAGGGCTAGCCAGAGTATAGGTACTAACATTCATTGCTAT | |
| | | | CAACAGCGAACCAACGGCTCACCCCGCTTGCTTATCAAATATGCCTCTGAGAGT | |
| | | | ATCTCTGGCATTCCATCCCGTTTTAGCGGTAGTGGTTCAGGAACAGATTTCACT | |
| | | | CTTTCAATTAACTCTGTAGAATCAGAAGACATAGCCGATTATTATTGTCAGCAA | |
| | | | AACAACAATTGGCCTACTACATTTGGAGCTGCTACAAAACTGGAACTCAAG | |
| | Panitumumab | VH | CAGGTACAACTGCAAGAGAGCGGACCTGGGTTGGTAAAGCCAAGTGAGACCCTG | 392 |
| | | | TCACTTACTTGCACACTATCAGGAGGAAGTGTCTCAAGTGGCGATTACTATTGG | |
| | | | ACTTCGATTCGACAAAGCCCCGGAAAAGGATTGGAATGGATCCGCCACATATAT | |
| | | | TATTCAGGAAATACCAACTATAATCCCAGTCTTAAATCTCGGCTTACCATTAGC | |
| | | | ATTGACACTTCCAAAACCCAATTTTCACTTAAACTGAGTTCTGTGACTGCTGCC | |
| | | | GACACTCCTATATACTACTGCGTCCGCGACCGGGTCACTGGGGCTTTTGATATA | |
| | | | TGGGGACAAGGAACTATGGTTACTGTATCATCT | |
| | | | GATATTCAAATGACCCAATCTCCTTCATCACTCTCAGCATCAGTCGCGGATAGA | 393 |
| | | | GTGACAATAACTTGCCAAGCAAGCCAGGATATATCTAATTATCTCAACTCGTAT | |
| | | | CAACAAAAGCCAGGTAAAGCTCCAAAGTTGTTGACTTACGATGCCTCTAATCTC | |
| | | | GAGACCGGCGTACCAAGTAGGTTCAGTGGTTCAGGAAGTGGGACTGATTTCACT | |
| | | | TTTACCATTTCTTCTCTCCAGCCCGAAGACATACCAACTTATTTCTGTCAACAC | |
| | | | TTCGATCATCTTCCTACAAAGTTGAAATCAAG | |
| | Necitumumab | VH | CAGGTACAACTCCAGGAATCCGGTCCTGGTCTCGTAAAGCCATGCCAAACATTG | 394 |
| | | | AGTTTGACCTGCACAGTCACTGGTGGCAGTATTTCCTCAGCCGATTATTACTGG | |
| | | | AGTTGGATACGCCAGCCACCCGGTAAGGGATTGGAATGGATCGGTTATATATAC | |
| | | | TACTCTGGATCTACAGACTATAATCCTTCCCTGAAGTCCAGGGTTACCATGAGT | |
| | | | GTTGACACATCCAAAAACCAGTTCTCCTTGAAGGTCAACTCTGTGACCGCTGCC | |
| | | | GATACTGCCCTTTATTATTGTGCACCTGTATCTATCTTCGGAGTAGGCACCTTT | |
| | | | GACTATTGGGTCAAGGCACCCTGGTCACCGTGTCATCT | |
| | | | GAGATACTAATGACACAGTCACCTCCTACCCTTTCCCTCAGCCCTCGCGAACGC | 395 |
| | | | GCCACCTTGTCTTGCAGGGCTAGCCAAAGTGTATCCTCTTATTTGGCATGGTAT | |
| | | | CAGCAGAAACCAGGGCAAGCCCCACGGCTCCTGATTTATGATGCCTCCAATCGA | |
| | | | GCCACCGGGATTCCTCCCCGATTGAGCGGATCAGGCAGTCGGACTGATTTTACA | |
| | | | TTGACCATTAGCTCTCTGGAGCCTGAGGACTTTGCCGTGTATTATTGTCATCAA | |
| | | | TATGGGAGTACTCCCCTCACATTTGGCGGCGGGACAAAAGCAGAAATTAAA | |
| | Imgatuzumab | VH | CAAGTACAGTTCGTACAAAGTGGGGCCGAGGTCAAGAAACCTGGTTCTAGCGTA | 396 |
| | | | AAGGTTAGCTGCAAGGCAAGCGGCTTCACCTTCACCGATTACAAAATTCATTGG | |
| | | | GTCCCTCAAGCCCCCCGTCACGGCCTTGAATCGATGCGTTATTTCAACCCCAAC | |
| | | | TCTGGTTATTCTACCTATGCACAGAAGTTCCAGGGCCGCCTCACTATAACTGCA | |
| | | | GACAAGTCCACAAGTACAGCCTATATCGAACTGTCCAGTTTGCGTAGCGAGGAT | |
| | | | ACTGCCGTATATTACTGTGCTAGACTCTCACCCGGCGGATATTACGTTATGGAT | |
| | | | GCATGGGGCCAGGGCACCACCGTGACCGTGAGCAGC | |
| | | VL | GACATCCAAATGACACAGTCTCCTTCCTCACTCTCCGCTTCTGTAGGAGATAGA | 397 |
| | | | GTGACAATAACATGCCGTGCTTCACAAGGTATCAATAACTACCTCAATTGGTAT | |
| | | | CAACAGAAACCTGGTAAAGCTCCAAAGCGCCTTATCTATAATACCAACAATCTC | |
| | | | CAGACTGGTGTTCCAAGTAGGTTCTCCGGGAGCGGTAGTGCGACTGAGTTTACA | |
| | | | TTGACAATTTCATCATTGCAACCCGAAGATTTTGCCACCTACTATTGCTTGCAG | |
| | | | CATAATAGTTTTCCCACTTTCGGTCAGGGAACTAAGCTCGAAATAAKA | |
| | Depatuxizumab | VH | CAGGTACAACTCCAAGAATCTGGACCTGGGCTGGTAAAACCATCTCAGACCTTG | 398 |
| | | | AGTCTCACTTGCACTGTGTCCCGTTACTCTATATCTAGCGATTTT [symbol] CATGGAAT | |
| | | | TGGATTCGCCAGCCACCCGGTAAGGGACTGGAGTGGATGGGTTACATTTCATAC | |
| | | | AGTGGAAACACACGTTACCAACCCAGTCTCAAGAGCCGGATAACCATTAGTAGC | |
| | | | GACACAAGCAAAAACCAATTTTTCCTCAAACTGAATTCAGTGACTCCTGCCGAC | |
| | | | ACAGCCACTTACTATTGTGTCACAGCCGGTCGGGGGTTCCCTTACTGGGGACAG | |
| | | | GGTACTCTCCTTACTGTAAGTTCT | |
| | | | GACATACAAATGACTCAATCACCTAGCTCCATGTCTGTTTCCGTTGGCGACCGC | 399 |
| | | | GTAACCATCACCTGTCATTCCTCACAGGATATAAATTCCAATATTGGGTGGCTT | |
| | | | CAGCAGAAACCAGGTAAATCATTCAAGGCCCTTATTTATCACCGGACCAACCTT | |
| | | | GATGACGGTGTCCCCTCTCGCTTTAGCGGTTCTGGATCAGGTACAGATTATACC | |
| | | | CTGACCATCTCTTCTCTCCAGCCCGAGGATTTCGCTACTTACTACTGTGTGCAA | |
| | | | TACGCACAGTTCCCCTGGGACATTCGGCGGTGGAACTAAACTGGAGATAAAG | |
| | Losatuxizumab | VH | GAAGTTCAGTTGCAAGAAAGTGGCCCCGGACTTGTTAAACCATCCCAGACCTTG | 400 |
| | | | AGCCTGACTTGCACCGTTTCTGCCTATTCTATTTCCCGCGACTTCGCATCGAAC | |
| | | | TGGATACGCCAACCTCCCGGCAAGGGCTTGGAGTGGATGGGGTATATCTCCTAT | |
| | | | AACGGCAATACACGCTATCAACCTAGTTTGAAGAGCCGCATAACCATCAGTAGA | |
| | | | GACACAAGTAAAAACCAATTCTTCTTGAAACTGAATAGTGTGACCGCTGCAGAC | |
| | | | ACTGCAACCTATTACTGCGTCACAGCCAGTCGGGGGTTTCCATATTGCGGTCAA | |
| | | | CGTACACTTGTAACCGTGTCTTCA | |
| | | VL | GATATACAAATGACCCACTCCCCTTCCTCCATGTCTGTCAGCGTCGCTGATAGG | 401 |
| | | | GTTACCATAACTTGTCATAGCAGCCAAGACATAAACAGCAATATAGGTTCGCTG | |
| | | | CAACAAAAGCCCCGTAAATCTTTCAAGGGGTTGATTTACCACGGAACTAACETC | |
| | | | GATGATGGTGTGCCCAGTCGCTTTTCAGGTAGTGGGTCTGGAACAGACTACACC | |
| | | | CTGACAATCTCCAGTTTGCAACCAGAAGACTTTGCTACTTATTATTGTGTACAG | |
| | | | TACCCTCAATTTCCTTGGACTTTCGGGGGAGGAACCAAACTTGAGATCAAG | |
| EGFR | Etevritamab | VH | CAAGTCCAACTCGTGGAGTCTGGAGGCGGTGTGGTCCAAAGCGGGAGATCCTTG | 402 |
| VIII | | | CGGCTGTCTTGCGCAGCATCAGGGTTTACATTTAGGAACTACGGCATGCAGTGG | |
| | | | GTTAGACAGGCACCCGGTAAGTGTCTTGAATGGGTAGCAGTAATCTGGTATGAT | |
| | | | GACAACTCAAAGAACACTTTGTACCTCCAAATGAACTCTCTGAGGGCCGAGGAT | |
| | | | ACAGCTGTATATTACTCTGCTAGGGATGGCTATGACATACTCACCCGCAACCCA | |
| | | | CGGGATTTCCATTACTGGGGGCAAGGGACATTGGTAACAGTTTCATCT | |
| | | VL | GATACAGTAATGACCCAGACTCCTCTGAGTAGTCATCTTACACTCGGCCAGCCT | 403 |
| | | | GCCTCTATCTCCTGTCGTTCAAGTCAGAGTCTGGTGCACTCAGACGGAAACACC | |
| | | | TACCTTTCATGGTTGCAACAGCGTGGTGGTCAACCTCCTCGATTGTTGATATAC | |
| | | | AGAATTAGTCGGCGTTTTTCTGGCGTCCCAGATCGCTTCTCCGGCTCAGGTGCC | |
| | | | GGAACAGACTTCACCCTTGAGATCAGTCGAGTGGAGGCTGAGGATGTGGGCGTA | |
| | | | TATTACTGTATGCAAAGCACTCACGTGCCAAGGACATTCGGGTGTGGAACCAAG | |
| | | | GTAGAGATCAAA | |
| | AMG-595 | VH | CAGGTGCAGTTGGTTGAGAGCGGCGGAGGTGTGGTACAGCCTGGAAGGAGTCTG | 404 |
| | | | CGACTCTCATGTGCCGCCTCCGGTTTTACATTTTCTTCCTACGGCATGCATTGG | |
| | | | GTTCGACAAGCACCAGGCAAGGGATTGGAATGGGTCGCAGTAATTTCGTACGAC | |
| | | | GGGTCAAATAAATACTATGTTGACAGCGTGAAAGGGCGATTCACTATCAGTCGT | |
| | | | GACAATAGTAAAAACACTTTGTATCTGCAAATGAATAGTCTGAGGGCAGAGGAC | |
| | | | ACAGCAGTCTATTACTGTGCTCGTGATGGTTGGCAACAGCTTGCCCCTTTTGAC | |
| | | | TATTGGGGACAAGGAACTTTGGTAACAGTAAGCAGC | |
| | | VL | GACATTGTTATGACACAAACACCTTTGTCCTCACCCGTAACACTGGGCCAACCT | 405 |
| | | | GCCTCCATATCCTGCCGAAGCAGTCAATCACTTGTCCACTCTGATGCGAATACC | |
| | | | TACTTGTCATGGCTGCATCAGCGACCAGGTCAGCCTCCAAGGCTTCTCATATAT | |
| | | | AAAATATCCAATCGTTTTTCAGGTGTTCCCGACCGTTTCTCCGGCTCAGGGGCC | |
| | | | GGAACCGCCTTCACTCTCAAAATATCCAGAGTGGAGGCTGAAGACGTAGGTGTA | |
| | | | TATTATTGTATGCAAGCCACCCAGTTGCCACGCACCTTTGGGCAAGGGACTAAA | |
| | | | GTAGAAATAAAG | |
| PD-L1 | Atezolizumab | VH | GAAGTTCAACTGGTGGAGTCTGGAGGGGGTCTCGTCCAGCCCGGCGGGAGCTTG | 406 |
| | | | AGACTGTCTTGTGCTGCATCOGGOTTTACCTTTAGCGATTOCTGGATACACTGG | |
| | | | GTAAGACAAGCACCTGGTAAAGGGTTGGAATGGGTGGCATGOATTTOCCCATAC | |
| | | | GGGGGATCAACCTATTATGCTGATAGCGTGAAGGGCCGGTTTACTATTTCAGCC | |
| | | | GACACAAGCAAGAACACCGCCTACCTCCAAATGAATTCTCTGAGGGCCGAGGAC | |
| | | | ACAGCAGTCTATTACTGTGCTAGGAGGCATTGGCCCGGCGGATTCGACTACTGG | |
| | | | GGGCAGGGCACTCTGGTCACAGTAAGCTCA | |
| | | VL | GACATCCAAATGACACAGTCTCCAAGCTCTCTCTCCGCAAGCGTGGGCGATCGG | 407 |
| | | | GTCACAATAACATGCCGCGCCTCACAAGACGTGTCTACAGCCGTTGCTTGGTAT | |
| | | | CAGCAAAAGCCCGGAAAAGCTCCCAAACTGCTCATTTATTCAGCCAGCTTTCTC | |
| | | | TACAGCGGGGTGCCAAGCCGGTTCAGTGGGAGCGGAAGTGGCACCCACTTTACT | |
| | | | CTTACCATCTCCTCTCTGCAACCCGAAGACTTCGCCACCTATTACTGCCAGCAA | |
| | | | TACCTGTATCATCCAGCTACTTTCGGGCAAGGGACCAAGGTTGAAATCAAA | |
| | Avelumab | VH | GAGGTTCAGCTTCTGGAGTCAGGAGGTGGTCTTGTTCAACCCGGAGGGTCTCTC | 408 |
| | | | CGTTTGTCCTGCGCCGCAAGTGGGTTCACATTTTCCAGTTATATCATGATGTGG | |
| | | | GTGAGGCAAGCCCCTGGGAAAGGATTGGAGTGGGTCTCATCTATTTATCCATCT | |
| | | | GGAGGTATTACATTCTATGCCGACACAGTTAAGGGCAGGTTTACTATAAGCCGC | |
| | | | GATAATTCCAAAAACACCCTGTACCTGCAAATGAATTCACTGCGAGCCGAGGAC | |
| | | | ACTGCTGTCTACTATTGCGCCAGAATCAAACTGGGGACAGTAACAACTGTAGAC | |
| | | | TATTGGGGGCAGGGGACTTTGGTCACTGTATCATCA | |
| | | VL | CAGTCAGCTCTGACACAGCCCGCATCTGTCTCTGGGAGTCCCGGACAGTCAATA | 409 |
| | | | ACTATATCATGCACAGGGACCTCCTCTGATGTTGGGGGATACAATTACGTGTCC | |
| | | | TGGTATCACCAGCACCCOOGCAAGGOCCCCAAACTCATGATATACGACGTTTCA | |
| | | | AATCGTCCTTCAGGCGTGTCTAACAGATTTTCCGGTTCTAAATCAGGCAATACC | |
| | | | GCTTCCTTGACTATCTCAGGACTTCAAGCAGAGGATGAAGCAGACTATTACTGC | |
| | | | TCAAGCTATACCTCCAGTTCTACTAGAGTGTTCGGTACAGGAACTAAGGTAACA | |
| | | | GTGCTG | |
| | Durvalumab | VH | GAGGTTCAGCTOGTOGAGAGTGGGGGGGGACTTGTTCAGCCAGGCGGTTCATTG | 410 |
| | | | CGGTTGTCTTGTGCCGCCTCAGGATTTACATTCTCAAGATACTGGATGAGCTGG | |
| | | | GTGAGGCAAGCACCCGGTAAAGGTCTCGAGTGGGTAGCTAATATCAAACAAGAT | |
| | | | GGGAGCGAGAAGTATTATGTTGACAGCGTGAAGGGTCGCTTTACCATATCAAGG | |
| | | | GATAACGCTAAGAACTCCCTTTATCTTCAGATGAATAGTCTCCGCGCTGAGGAC | |
| | | | ACCGCAGTATATTACTGTGCTAGGGAAGGAGGGTGGTTTGGGGAATTGGCATTC | |
| | | | GATGGACTGGGGTCAGGGTACTCTCGTTACAGTCAGTTCC | |
| | | VL | GAAATCGTTTTGACACAGAGCCCTGGGACACTGTCCTTGAGCCCAGGAGAACGC | 411 |
| | | | GCAACCCTCTCCTGCCGTGCAAGTCAGCGTGTTTCCTCATCTTAGGTTGCTTGG | |
| | | | TATCAACAAAAGCCAGGGCAGGCTCCTAGACTGCTTATCTATGACGCTTCTAGC | |
| | | | AGAGCTACTGGGATACCAGATAGGTTTTCCGGGTCTGGTTCAGGCACAGACTTC | |
| | | | ACCCTCACCATATCTCGACTCGAACCTGAGGATTTTGCAGTGTACTATTGTCAA | |
| HER2 | Trastuzumab | VH | GAAGTGCAGCTGGTCGAAAGTGGCGGTGGACTTGTGCAACCTGGCGGTAGCCTC | 412 |
| | | | CGTCTCAGCTGCGCTGCAAGTGGGTTCAACATCAAGGACACTTATATTCATTGG | |
| | | | GTCCGACAGCCACCTGGGAAAGGTTTGGAGTGGGTCGCACGGATCTATCCCACT | |
| | | | AATGGTTACACAAGATATGCCGATTCAGTAAAAGGCCGGTTTACAATCAGCGCA | |
| | | | GATACTTCAAAAAACACTGCCTATCTTCAAATGAACTCACTTCGAGCAGAAGAC | |
| | | | ACAGCCGTCTATTATTGTAGTCGTTGGGGAGGCGACGGCTTTTATGCTATGGAC | |
| | | | TACTGGGGACAAGGAACTCTGGTCACAGTTTCATCA | |
| | | VL | GATATTCAGATGACTCAGAGTCCTAGTTCCCTCAGCGCCTCCGTAGGCGACAGA | 413 |
| | | | GTTACAATAACTTGCCGAGCAAGCCAAGACGTAAACACTGCAGTCGCCTGGTAC | |
| | | | CAACAGAAACCAGGCAAAGCTCCAAAACTCTTGATTTACAGTGCTTCCTTCCTT | |
| | | | TATAGTGGCGTTCCAAGCCGCTTCAGCGGCAGCCGCTCTGGCACCGACTTCACT | |
| | | | CTCACTATTTCTTCCTTGCAACCTGAAGACTTCGCCACTTATTATTGCCAGCAA | |
| | | | CACTACACAACACCCCCAACATTCGGACAGGGCACAAAGGTAGAAATAAAA | |
| | Pertazumab | VH | GAAGTGCAACTGGTGGAGTCTGGTGGTGGATTGGTGCAGCCAGGCGGTTCTCTG | 414 |
| | | | CGACTTAGTTGTGCAGCCTCCGGCTTTACCTTCACTGATTATACAATGGACTGG | |
| | | | GTTCGGCAGGCACCCGGTAAGGGGCTTGAGTCGCTCCCCGACGTCAATCCTAAT | |
| | | | TCAGGGGGAAGTATTTATAACCAAGGCTTCAAGGGTCGATTTACATTGTCCGTA | |
| | | | GATCGTAGTAMAAATACCCTCTACCTTCAAATGAACTCCCTGAGGGCAGACGAT | |
| | | | ACCGCAGTCTACTACTGCGCTCGTAACCTGGGGCCTAGTTTTTATTTCGATTAT | |
| | | | TGGGGCCAAGGCACATTGGTAACTGTGTCTTCA | |
| | | VL | GATATACAAATGACACAATCTCCTAGTTCATTGAGTGCCTCAGTCGGCGACCGA | 415 |
| | | | GTCACTATAACTTGTAAAGCAAGCCAAGATGTTAGCATTGGCGTAGCTTGGTAT | |
| | | | CAGCAGAAACCTGGAAAAGCACCAAAAGTGCTTATCTACTCCGCTAGTTACCGT | |
| | | | TACACCGGAGTTCCCTCAAGGTTTTCTGGCAGCGGAAGTGGGACTGACTTCACT | |
| | | | CTGACTATTTCTTCACTTCAGCCAGAAGACTTCGCTACTTATTACTGTCAGCAG | |
| | | | TACTATATCTATCCCTATACATTTGGACAAGGAACCAAAGTTGAGATTAAA | |
| c-Met | Onartuzumab | VH | GAAGTGCAACTGGTAGAATCTGGAGGGGGTCTTGTTCAACCTGGGGGCAGTCTC | 416 |
| | | | AGGCTGTCATGTCCAGCAAGTGGATATACATTTACTTCCTATTGGCTCCATTGG | |
| | | | GTACGACAAGCACCTGGGAAAGGGCTGGAATGGGTTGGTATGATCGACCCATCA | |
| | | | AACTCTGACACCCGCTTTAATCCAAATTTTAAAGACCGCTTTACAATATCCGCA | |
| | | | GATACAAGTAAGAACACCGCATATCTCCAGATGAACAGCCTGCGTGCAGAGGAT | |
| | | | ACCGCAGTGTACTACTGTGCAACCTACCGGTCCTATGTAACCCCTCTCGACTAT | |
| | | | TGGGGTCAAGGCACACTTGTCACCGTGAGTAGC | |
| | | VL | GACAATACAAATGACCCAATCACCAAGTTCCCTTTCTGCTTCAGTCGGTGATCG | 417 |
| | | | GTGACAATAACATGCAAATCATCTCAAAGTCTCTTGTACACAAGCAGCCAAAAA | |
| | | | AATTATCTTGCTTGGTACCAGCAGAAGCCAGGGAAAGCACCAAAACTGCTGATC | |
| | | | TACTGGGCTTCAACAAGAGAATCCGGGGTGCCCAGCCGCTTTTCCGGTTCGGGC | |
| | | | AGTGGAACTGATTTCACCCTCACTATTTCCTCATTGCAACCCGAGGACTTCGCA | |
| | | | ACCTATTACTGTCAACAGTATTACGCCTACCCTTGGACATTTGGACAAGGAACT | |
| | | | AAGGTTGAAATTAAA | |
| | Emibetuzumab | VH | CAAGTACAGCTCGTCCAATCCGGCGCTGAAGTCAAGAAGCCCGGAGCTTCCGTT | 418 |
| | | | AAAGTTTCCTCCAAAGCCAGCGGCTACACTTTCACTGATTACTATATGCACTGG | |
| | | | GTTAGACAAGCACCCGGGCAGGGTCTCGAATGGATGGGTAGGGTTAATCCAAAT | |
| | | | CGCAGGGGAACTACTTACAACCAGAAATTTGAGGGGAGGGTTACCATGACTACC | |
| | | | GATACCAGCACATCTACTGCATATATGGAGCTGCGTTCTCTGAGGAGTGATGAT | |
| | | | ACAGCAGTGTACTATTGTGCCCGCGCTAACTGGTTGGACTACTGGGGGCAAGGT | |
| | | | ACAACTGTCACAGTATCTAGC | |
| | | VL | GACATTCAAATGACACAAAAGTCCATCCTCTCTCAGTGCTTCAGTGGGGACCGA | 419 |
| | | | GTAACCATAACATCCAGTGTCTCAAGTAGCGTGAGTAGCATCTACCTCCATTGG | |
| | | | TATCAGCAGAAACCTGGCAAGGCACCCAAACTCCTCATTTATTCCACAAGTAAT | |
| | | | CTTGCTTCCGGCGTACCTTCTACGTTTAGCGGGTCCGGCTCTGGCACCGATTTC | |
| | | | ACCCTCACTATTAGCTCCTTGCAACCTGAGGACTTTGCTACTTACTATTGTCAG | |
| | | | GTTTATTCCGGTTACCCCCTCACATTCGGTGGAGGAACCAAGGTAGAGATTAAG | |
| | Telisotuzumab | VH | CAGGTCCAGTTGGTACAGTCAGGGGCAGAAGTTAAGAAACCAGGCGCTTCTGTA | 420 |
| | | | AAGGTTAGTTGCAAGGCAAGCGGGTATATATTCACAGCCTATACCATGCATTGG | |
| | | | GTGCGTCAAGCTCCTGGGCAAGGATTGGAGTGGATGGGCTGGATCAAGCCCAAC | |
| | | | AATGGTCTGGCCAACTACGCACAGAAGTTCCAAGGTCGTGTAACCATGACCAGG | |
| | | | GACACTTCAATAAGCACCGCCTACATGGAATTGAGCAGACTTCGATCAGATGAT | |
| | | | ACAGCAGTTTACTATTGCGCTAGGAGTGAAATTACCACAGAGTTTGATTACTGG | |
| | | | GGCCAAGGAACTCTGGTGACTGTTTCCAGT | |
| | | VL | GATATCGTTATGACACAGTCCCCCGACAGCCTGGCTGTCAGTCTCGGGGAGAGA | 421 |
| | | | GCAACTATAAACTGTAAAAGCAGTGAATCCGTCGATTCATACGCAAACAGTTTT | |
| | | | CTGCATTGGTATCAGCAAAAACCCGGCCAGCCACCCAAACTGCTCATATATCGG | |
| | | | GCTAGTACACGTGAGTCAGGCGTACCAGACCGCTTTAGCGGATCAGGAAGTGGG | |
| | | | ACAGACTTTACCTTGACCATTAGCTCACTTCAGGCTGAGGACGTTGCAGTTTAC | |
| | | | TACTGCCAACAAACTAAGGANGACCCACTCACATTCGGCGGAGGAACTAAGGTC | |
| | | | GAGATTAAG | |
| TROP-2 | Datopotamab | VH | CAGGTGCAGTTGGTCCAGTCCGGTGCAGAGGTAAAGATACCAGGCGCTTCCGTT | 422 |
| | | | AAAGTATCCTGTAAAGCTAGCGGTTATACTTTCACTACTGCCGGAATGCAGTGG | |
| | | | GTGCGACAGGCTCCTGGTCAGGGTCTCGAATGGATGGGATGGATCAATAGTCAC | |
| | | | TCAGGAGTGCCAAAGTATGCTGAAGATTTCAAGGGGCGTGTTACTATCTCCGCT | |
| | | | GACACTCCACATCTACTGCTTATCTTCAGCTCTCTAGCCTGAAGTCAGAAGAT | |
| | | VL | GATATACAGATGACCCAATCACCCTCTTCTTTGAGTGCATCAGTAGGTCATCGT | 493 |
| | | | GTTACAATTACCTCTAAACCTAGTCAAGACGTGTCTACTGCTGTCGCCTCGTAT | |
| | | | CAACAGAAACCAGGCAAAGCACCAAAACTTTTGATTTATTCTGCTTCTTATAGA | |
| | | | TATACAGGCGTCCCCAGCCGGTTTTCAGGTTCCGGTTCCGGGACCGACTTTACC | |
| | | | CTCACTATCAGCTCTCTTCAGCCTGAGGACTTCGCCGTTTATTATTGCCAGCAG | |
| | | | CACTATATCACCCACTGACTTTTGGGCAAGGGACTAAGCTGGAGATCAAA | |
| | Sacirazum ab | VH | CAAGTACAGCTTCAGCAATCAGGCTCTGAACTCAAGAAACCTGGTGCCAGCGTG | 424 |
| | | | AAAGTATCCTCTAAGGCATCCCGCTACACTTTTACAKACTATGGAATGAATTGG | |
| | | | GTAAAACAAGCCCCCGGACAAGGGCTTAAATGGATGGCTTGGATATATACTTAC | |
| | | | ACCGGAGAACCTACATATACAGACGACTTTAAAGGGCGATTCGCTTTCTCCCTT | |
| | | | ACCGGAGAACCTACATATACAGACGACTTTAAAGGGCGATTCGCTTTCTCCCTT | |
| | | | GACACAAGTGTAAGCACAGCCTACCTGCAAATCAGCAGTCTCAAAGCCGACGAT | |
| | | | ACAGCCGTGTACTTTTGCGCCAGGGGTCGCTTCGGCTCCACCTATTGGTACTTC | |
| | | | GATGTCTGGGGACAGGGCAGCCTTGTAACTGTATCTAGT | |
| | | VL | GATATTCAGTTGACACAGAGCCCTAGCTCACTTTCAGCCTCTGTGGGTGATAGG | 425 |
| | | | GTTAGTATTACCTGTAAGGCATCCCAAGACGTTAGCATTGCCGTCGCCTGGTAC | |
| | | | CAGCAGAAACCCGGAAAAGCTCCAAAACTGCTCATCTACTCAGCATCCTATCGT | |
| | | | TATACAGCTGTACCTGACAGGTTCTCTGCCTCCGGGAGCGGTACCGATTTTACT | |
| | | | TTGACCATTTCAAGTTTGCAACCAGAAGACTTCGCTGTTTACTACTGCCAGCAG | |
| | | | CATTACATTACTCCACTCACATTCGGAGCAGGGACAAAGGTCGTAATCAAA | |
| DLL3 | Rovalpituzumab | VH | CAAGTCCAGCTTGTTCAAAGTGGGGCTGAAGTGAAGAAACCAGGGGCTAGTGTT | 426 |
| | | | AAAGTGAGCTGTAAGGCATCAGGATACACTTTCACAAACTACGGAATGAATTGG | |
| | | | GTTCGCCAAGCACCTGGTCAAGGCTTGGAATCGATGCGTTGGATTAACACATAT | |
| | | | ACAGGTGAGCCAACTTACCCCGATGATTTCAAGGGGCGAGTTACCATGACTACC | |
| | | | GACACCTCAACATCCACTGCATACATGGAGCTTCGCTCACTCCGAAGCGATGAT | |
| | | | ACTGCAGTTTACTATTGCGCTCCCATCCGTGACTCATCACCTAGCGACTACTGG | |
| | | | GGCCAAGCTACATTGCTAACAGTTTCTTCA | |
| | | VL | GAGATCGTGATGACCCAGAGTCCCCCTACTCTCTCAGTGAGTCCTGGTGAACGT | 427 |
| | | | GCTACACTGTCTTGTAAGGCCAGTCAGTCCGTCTCAAACGATGTCGTTTGGTAT | |
| | | | CAGGAAAAGCCAGGACAAGCCCCCAGACTCCTGATATACTACGCCAGTAATCGC | |
| | | | TATACTCGAATCCCCGCTAGATTCAGTCGGAGTGGAAGCGGAACTGAATTTACC | |
| | | | TTGACTATATCCTCATTGCAAAGCGAAGACTTTGCCGTTTACTATTGTCAACAA | |
| | | | GACTACACCTCTCCTTGGACCTTCGGACAAGGTACAAAACTTGAAATCAAG | |
| | Tarlatamab | VH | CAAGTACAGCTCCAAGAAACTGGCCCCCGATTGGTGAAGCCATCCGAAACACTT | 428 |
| | | | TCCCTTACCTGCACTGTCTCCGGGGGCTCCATCAGTAGTTATTACTGGAGTTGG | |
| | | | ATACGCCAACCACCCGGTAAGTGTCTGGAGTGGATAGGTTATGTGTATTACTCA | |
| | | | GGCACAACCAATTATAATCCATCCTTGAAAAGCCGGGTAACCATCTCAGTAGAT | |
| | | | ACCAGCAAAAACCAGTTCTCCCTGAAACTGTCCAGTGTTACTGCTGCTGATACC | |
| | | | GCCCTATATTATTGTGCATCCATTGCAGTGACAGGGTTTTATTTTGACTATTCG | |
| | | | GGCCAGGGTACTTTGGTAACCGGTATCTTCA | |
| | | VL | GAGATCGTCCTGACCCAAAGCCCAGGTACTCTTTCCCTCAGCCCAGGCGAAAGG | 429 |
| | | | GTCACTCTGTCATGCAGGGCTAGTCAAAGAGTCAACAATAATTACCTCGCATGG | |
| | | | TATCAACAAAGACCCGGACAGGCTCCACGCCTGCTCATATATCGAGCAAGTAGC | |
| | | | CGAGCTACTCGCATTCCCGATAGATTCAGTGGATCTGGATCTGGGACCGATTTT | |
| | | | ACTCTGACAATAAGTCGTCTTGAACCTGAAGATTTTGCAGTATACTATTGTCAG | |
| | | | CAATATGACAGGAGCCCCCTGACATTCGGGTGCGGTACTAAGCTGGAAATCAAA | |
| BCMA | Belantamab | VH | CAAGTCCAACTGGTCCAGTCAGGCGCAGAAGTTAAAAAGCCTGGCAGCAGTGTG | 430 |
| | | | AAGGTGTCTTGTAAGGCAAGCGGCGGTACATTTAGTAATTATTGGATGCACTGG | |
| | | | GTACGGCAGGCTCCCGGCCAAGGGCTTGAATGGATGGGCGCCACATACCGAGGT | |
| | | | CATTCAGACACCTATTACAACCAGAAATTCAAGGGGCGCGTGACCATTACAGCA | |
| | | | GATAAATCAACTTCTACAGCCTACATGGAACTCAGCTCCCTCCGGTATGAGGAT | |
| | | | ACAGCAGTCTACTACTGTGCTCGCGGAGCCATTTACGATGGGTATGATGTGCTG | |
| | | | GATAATTGGGGCAGGGCACACTCGTGAACCGTAAGTAGT | |
| | | VL | GATATACAGATGACCCACTCACCATCCAGCCTTAGTGCATCCGTCGCGGATCGG | 431 |
| | | | GTGACTATTACTTGCTCCGCTTCTCAAGATATTTCAAACTATCTGAATTGGTAT | |
| | | | CAGCAAAAGCCTGGGAAGGCCCCAAAATTGCTGATCTATTACACTTCAAATTTG | |
| | | | CACTCAGGGGTTCCCTCTCGCTTCAGCGGAAGCGGAAGCGGTACTGATTTTACC | |
| | | | TTGACTATCTCTAGCCTCCAGCCAGAGGACTTTGCTACCTACTACTGCCAACAC | |
| | | | TACAGGAAACTCCCATGGACTTTTGGACAAGGCACCAAGCTCGAAATTAAG | |
| LIV-1 | Ladiratuzumab | VH | CAAGTTCAGTTCGTTCAATCTGGGGCCGAAGTCAAAAAACCTGGCGCTTCAGTT | 432 |
| | | | AAAGTTAGCTGCAAAGCAAGCGGTCTCACTATAGAAGACTATTATATGCACTGG | |
| | | | GTCAGACAGGCTCCAGGGCAAGGGCTTGAGTGGATGGGGTGGATAGATCCAGAG | |
| | | | AATGGGGACACCGADTATGCACCCAAATTCCAGGGGCGTGTAACCATGACCCGA | |
| | | | GACACTTCAATAAATACTGCATACATGGAACTCTCCCGGCTCCGGAGCGACGAT | |
| | | | ACAGCCGTGTATTACTGTGCTGTCCACAACGCCCACTACCGCACATCGTTTCCA | |
| | | | TATTGGGGGCAGGGAACTCTTGTTACTGTTTCTTCA | |
| | | VL | GACGTAGTGATGACTCAGTCTCCACTGTCCCTGCCAGTGACATTGCGCCAACCT | 433 |
| | | | GCAAGTATTTCATGCAGATCAAGTCAATCTCTCCTGCACAGTAGCGGCAACACA | |
| | | | TACTTGGAGTGGTATCAACAACGCCCAGGTCAATCACCCAGGCCACTGATATAT | |
| | | | AAAATCTCAACTCGATTCAGCGGTGTTCCCGACAGGTTCTCAGGATCTGGCTCC | |
| | | | GGCACTGATTTTACCTTGAAGATCTCACGAGTGGAAGCTGAGGATGTGGGAGTA | |
| | | | TATTACTGTTTCCAAGCTTCACATGTCCCTTATACTTTTGGTGGAGGAACTAAG | |
| | | | GTAGAGATCAAG | |
| GPC-3 | Codrituzumab | VH | CAGGTGCAACTCGTTCAAAGCGGGGCCGAGGTGAAGATACCAGGGGCCTCAGTT | 434 |
| | | | AAGGTGAGTTGCAAGGCAAGTGGATACACTTTCACCGATTATGAAATGCATTGG | |
| | | | GTGCGTCAGGCCCCAGGACAAGGACTGGAGTGGATGGGCGCTCTCGATCCTAAG | |
| | | | ACTGGTGATACTGCTTACTCTCAAAAGTTCAAAGGCCGAGTCACCTTGACCGCC | |
| | | | GACAAGTCCACATCCACTGCATATATGGAATTGTCAAGTCTGACAAGCGAAGAT | |
| | | | ACAGCCGTCTACTACTGCACCGCCTTTTATAGCTATACATATTGGGGACAGGGG | |
| | | | ACCTTGGTTACTGTGTCATCT | |
| | | VL | GACGTGGTAATGACACAATCACCTTTGTCTCTTCCCGTAACCCCCGGTGAACCA | 435 |
| | | | GCCAGCATCTCATGCAGAAGCAGTCAGTCACTGGTACATTCCAACCGTAATACT | |
| | | | TATCTTCACTGGTACTTCCASAAGCCTGGGCAGTCTCCTCAACTTTTGATATAT | |
| | | | AAAGTGAGCAATCGGTTTAGCGGTGTCCCAGACCGCTTTTCTGGATCTGGAAGT | |
| | | | GGAACAGACTTTACTCTGAAAATAAGCAGAGTCGAGGCAGAAGATGTCGGAGTT | |
| | | | TACTACTGTAGCCAGAACACACACGTACCCCCAACCTTTGGACAGGGCACAAAG | |
| | | | TTGGAAATCAAG | |
| FGFR2 | Apratumab | VH | GAGGTACAACTGCTTGAATCTGGAGGAGGGTTGGTACAACCTGGTGCTTCACTG | 436 |
| | | | CGATTGTCCTGTGCACCCTCAGGCTTTACTTTCTCATCATATGCCATGTCCTGG | |
| | | | GTAAGGCAGGCACCTGGAAAAGGACTCGAATGGGTCTCAGCCATCTCCGGTTCA | |
| | | | GCCACATCAACTTACTATCCAGACTCTGTCAAAGGGCGCTTTACAATATCTAGG | |
| | | | GATAATTCAAAAAATACATTGTACTTGCAGATGAACAGTTTGCGTGCCGAAGAT | |
| | | | ACCGCAGTGTACTATTGCGCTAGGGTTCGATATAACTGGAACCATGGTGACTGG | |
| | | | TTTGACCCTTGGGGCCAAGGCACTGGTGACAGTGAGTTCC | |
| | | VL | CAGTCCGTCCTCACACAACCACCTAGTGCCTCTGGTACACCAGGACAACGTGTC | 437 |
| | | | ACAATTTCCTGCAGCGGGTCAAGTTCAAACATAGGGAATAACTATGTGTCATGG | |
| | | | TATCAACAACTTCCTGGTACTGCTCCAAAGCTCCTCATTTATGAAAACTATAAC | |
| | | | AGGCCCCCAGGTGTCCCAGATCGATTTTCACGATCAAAGTCCGGTACCTCAGCC | |
| | | | AGTTTGGCAATTAGTGGCCTTCGATCCGAAGATGAAGCAGATTACTACTGTTCA | |
| | | | TCCTGGGACGATTCTCTTAACTATTGGGTATTCGGCGGAGCCACTAAACTCACC | |
| | | | GTCCTT | |
| | Bemarituzumab | VH | CAAGTGCAGCTTGTTCAGAGTGGGGCTGAAGTCAAAAAGCCAGGCTCAAGCGTG | 438 |
| | | | AAACTCAGCTGCAAGGCCAGCCGTTACATCTTCACAACTTACAATGTTCACTGC | |
| | | | GTCAGACAAGCCCCTGGTCAGGCGCTTGAGTGGATÄGGATCAATCTACCCCGAT | |
| | | | AATGGGGACACCAGCTATAATCAAAACTTCAAAGGACGTGCAACAATCACAGCC | |
| | | | GACAAGTCAACTTCAACAGCCTACATGGAGCTTTCCAGCTTCCGATCCGAAGAT | |
| | | | ACTGCCGTATATTACTGTGCTAGAGGCGACTTCGCTTATTGGGGACAAGGTACT | |
| | | | TTGGTGACTGTTTCTTCT | |
| | | VL | GACATTCAAATGACCCAATCCCCCAGTTCCTTGAGTGCCTCCGTCGGAGATCGA | 439 |
| | | | GTTAGTATTACATGTAAAGCTAGTCAAGGGGTCAGTAACGACGTTGCTTGGTAC | |
| | | | CAGCAGAAGCCAGGTAAGGCTCCCAAGCTCCTGATATATAGCGCCTCATACCGC | |
| | | | TACACAGGTGTGCCTTCCCGGTTTAGTGGCTCAGGATCAGGGACAGATTTTACA | |
| | | | TTCACTATAAGCTCTTTGCAGCCCGAAGACATAGCCACATATTATTGCCAGCAG | |
| | | | CATTCCACTACTCCATACACATTTGGACAGGGAACAAAGCTGGAGATTAAG | |
| FGFR3 | Vofa [symbol] amab | VH | GAAGTACAGCTTGTCGAGAGCGGTGGCGGGCTTGTACAACCTGGTGGAAGCTTG | 440 |
| | | | CGGTTGAGTTGTGCAGCCAGCGGTTTCACATTTACTAGCACTGGAATATCATGG | |
| | | | GTCCGTCAAGCTCCAGGGAAAGGGCTGGAATGGGTTGGCCGAATATATCCCACC | |
| | | | AGTGGCTCTACCAACTACGCAGACTCAGTCAAGGGTCGCTTTACAATTTCTGCT | |
| | | | GACACAAGTAAGAAGACCGCATATTTGCAGATGAACTCACTGCGAGCCGAGGAT | |
| | | | ACCGCCGTTTACTACTGTGCAAGGACATACGGAATTTACGATCTTTACGTTGAT | |
| | | | TATACAGAGTATGTGATGGATTATTGGGGGCAGGGCACCCTCGTCACTGTGAGT | |
| | | | TCT | |
| | | VL | GACATCCAGATGACCCAGAGCCCCTCTTCTTTGTCAGCAAGCGTCGGAGACCGC | 441 |
| | | | GTTACCATTACTTGCCCTGCCTCTCAGGACGTCGACACCAGCCTTCCTTGCTAC | |
| | | | AAGCAGAAACCAGGAAAAGCCCCCAAGCTGCTCATCTATTCCGCTTCATTTCTC | |
| | | | TACAGCCGAGTGCCATCCCCTTTCTCCGGTTCAGGCTCTGGAACAGACTTCACT | |
| | | | CTGACTATAAGCAGTCTTCAACCCGAAGACTTCGCTACATACTATTGTCAGCAA | |
| | | | TCAACCGGACACCCACAGACATTCGGCCAGGGCACTAAAGTAGAGATTAAA | |
| VEGFR2 | Ramucirumab | VH | GAAGTTCAGCTTGTGCAGAGTGGCGGAGGGCTTGTGAAACCAGGAGGATCACTG | 442 |
| | | | AGGCTCTCCTGTGCAGCATCCGGTTTCACATTCAGCAGCTATAGTATGAACTGG | |
| | | | GTGCGTCAGGCTCCAGGGAAGGGACTGGAATGGGTCTCAAGCATTTCCTCCTCC | |
| | | | TCTTCATATATATATTACGCCGACAGTGTAAAAGGCGGCTTTACAATATCTCGG | |
| | | | GATAACGCTAAAAATAGCTTGTACCTTCAGATGAATTCACTGAGGGCTGAGGAC | |
| | | | ACTGCTGTGTACTACTGTGCAAGAGTCACCGACGCTTTTGATATTTGGGGTCAG | |
| | | | GGGACAATGGTGACCGTCTCCTCA | |
| | | VL | GATATACAAATGACCCAGTCTGGGAGTTCAGTATCCGCCAGCATAGGTGACCGC | 443 |
| | | | GTGACCATAACATGCCGGGCCAGCCAGGGAATTGATAATTGGTTGGGGTGGTAT | |
| | | | CAACAAAAGCCCGGAAAAGCTCCCAAGCTCCTTATCTATGATGCTTCTAACTTG | |
| | | | GATACAGGTGTACCCAGTCGATTTAGTGGCTCCGGGAGTGGGACTTATTTCACC | |
| | | | CTCACTATATCTTCTCTGCAAGCAGAAGACTTTGCAGTATATTTCTGTCAACAG | |
| | | | GCCAAAGCATTCCCTCCAACCTTCGCTGGGGGGACAAAGGTAGACATTAAA | |
| CD20 | Rituximab | VH | CAGGTCCAGCTCCAACAACCTGGAGCAGAATTGGTCAAGCCAGGGGCAAGCGTG | 444 |
| | | | AAGATGAGCTGCAAGGCAAGCGCCTATACTTTCACCTCCTACAATATGCATTGG | |
| | | | GTCAAACAAACTCCAGGTCGTGGGCTTGAGTGGATCGGGGCCATTTACCCAGGC | |
| | | | AACGCCGACACCTCATATAACCAAAAGTTTAAGGGAAAAGCCACTTTGACAGCA | |
| | | | GATAAAAGTAGTAGCACTCCATACATGCAACTGTCAAGTCTGACTAGCGAAGAT | |
| | | | AGTGCCGTATATTATTGCGCTAGGTCCACATATTACGCCGCTGATTGGTACTTC | |
| | | | AATGTTTGGGGAGCCGGGACTACAGTCACCGTATCCGCT | |
| | | VL | CAAATCGTCCTGTCTCAATCACCAGCAATTCTGAGTCCTAGTCCCGGAGAAAAA | 445 |
| | | | GTCACTATGACCTGTAGAGCCTCATCATCTGTTTCCTATATACATTGGTTTCAG | |
| | | | CAGAAACCTGGATCTTCTCCCAAGCCCTGGATTTATCCAACCTCTAACCTCGCA | |
| | | | AGTGGAGTCCCCGTGCGGTTTTCAGGCAGCGGTTCCGGTACAAGTTATTCCCTG | |
| | | | ACCATCAGCCGTGTGGAAGCAGAAGACGCCGCCACATACTACTGCCAACAGTGG | |
| | | | ACCTCARATCCTCCCACCTTTGGGGGAGGTACTAAACTTGAAATAAAA | |
| | Obinutuzumab | VH | CAAGTGCAACTGGTTCAAAGTGGAGCCGAGGTCAAAAAACCTGGTTCCTCCGTC | 446 |
| | | | AAAGTGTCTTGTAAAGCTTCAGGGTACGCATTCTCCTACTCCTCGATAAACTGG | |
| | | | GTGCGTCAGCCTCCTGGGCAAGCTCTGGAATGGATGGGCCGGATTTTCCCAGGA | |
| | | | GATGGCGACACAGACTACAATGGGAAGTTTAAGCGTCGGGTAACCATCACCGCT | |
| | | | GACAAGAGTACATCTACCCCCTATATGGAGCTTTCTTCACTTAGGACTGAGGAC | |
| | | | ACAGCAGTCTACTATTGTGCTCGAAATGTGTTTGACCGGTATTGCCTGGTGTAT | |
| | | | TGGGGCCAGGGTACCCTCGTAACACTATCATCA | |
| | | VL | GATATTGTTATGACACAAACACCACTGTCCCTCCCTGTTACACCCGGAGAGCCT | 447 |
| | | | GCTTCCATAAGTTGTCGATCCTCCAAATCACTTCTCCACTCAAATGGAATCACT | |
| | | | TATCTTTATTGGTATCTTCAGAAGCCAGGACAGTCCCCTCAACTGTTGATTTAT | |
| | | | CAGATGTCAAATTTGGTGAGTGGGGTGCCAGATAGGTTTTCTGGATCCGGTTCC | |
| | | | GGTACTGACTTTACATTGAAAATATCCCGAGTCGAAGCCGAAGACGTGGGCGTG | |
| | | | TACTATTGCGCTCAGAACCTTGAGCTGCCTTACACCTTTGGCGGTGGGACTAAA | |
| | | | GTGGAAATTAAG | |
| CD38 | Daratumumab | VH | GAGGTTCAGTTGCTGGAGAGCGGAGGGGGGCTTGTCCAACCAGGCGGTTCTCTG | 448 |
| | | | CGACTTTCTTGTGCAGTGTCTGGGTTTACCTTCAACAGCTTTGCCATGTCCTGG | |
| | | | GTGCGCCAAGCACCCGGAAAGGGACTGGAGTGGGTTAGCGCAATCTCTGGGTCA | |
| | | | GGGGGAGGGACTTATTATGCTGACTCTGTTAAGGGTAGATTTACAATCAGTCGC | |
| | | | GATAATAGTAAAAATACACTGTATCTTCAGATGAACTCTCTCAGAGCCGAGGAT | |
| | | | ACAGCCGTGTATTTCTGTGCCAAAGACAAGATCCTTTGGTTCGGAGAGCCTGTT | |
| | | | TTCGACTATTGGGGTCAAGGGACATTGGTGACAGTAAGCTCT | |
| | | VL | GAAATTGTTCTCACCCAGAGTCCAGCTACCCTGTCCCTGAGCCCCGGCGAGAGA | 449 |
| | | | GCAACCTTGAGTTGCCGAGCCTCTCAATCCGTCTCCTCCTATCTGGCCTGGTAC | |
| | | | CAACAAAAACCAGGCCAACCCCCCCGTTTGCTGATATACGACGCCAGTAACCGA | |
| | | | GCTACCGGCATACCCGCCCGCTTTAGCGGCTCTGGATCTGGTACAGATTTCACA | |
| | | | CTCACTATATCAAGTCTGGAACCTGAAGATTTCGCAGTCTATTATTGCCAACAA | |
| | | | CGGTCAAATTGGCCCCCTACATTTGGACAAGGGACCAAAGTGGAGATTAAG | |
| EphA2 | 1C1 | VH | GAGGTACAGTTGCTGGAGTCAGGAGGTGGATTGGTCCAACCCGGAGGATCTCTT | 450 |
| | | | CGTCTGTCCTGCGCCGCCTCAGGATTTACCTTCTCTCATTATATGATGGCATGG | |
| | | | GTACGTCAGGCTCCAGGCAAAGGTCTGGAATGGGTTAGTCGGATTGGTCCCTCA | |
| | | | GGGCGTCCTACCCATTATGCCGATTCTGTAAAGGGCCGTTTTACCATAAGCAGA | |
| | | | GACAACTCTAAGAACACCCTTTACCTTCAGATGAATAGCCTGAGGGCTGAGGAT | |
| | | | ACCGCAGTGTATTACTGCGCAGGCTATGACTCTCGGTACGACTATGTCGCCGTA | |
| | | | GCAGGACCTGCCGAGTATTTTCAACACTGGGGACAGGGGACCCTTGTCACAGTT | |
| | | | TCTAGT | |
| | | VL | GATATTCAAATGACACHAAGCCCAAGTTCCTTGTCCGCCTCAGTTGGTGATCGT | 451 |
| | | | GTGACAATAACCTGTCGGGCTTCACAATCCATATCTACATGGCTGGCTTGGTAC | |
| | | | CAGCAAAAGCCAGGTAAAGCCCCAAAACTCCTGATTTACAAGGCAAGTAACTTG | |
| | | | CATACTGGGCTACCCAGCCGTTTCTCTGCGTCACGCTCTCGGACAGAGTTTAGT | |
| | | | CTTACAATTTCTGGTCTGCAACCCGATGACTTCGCTACCTATTACTATCAACAA | |
| | | | TATAATAGTTATTCTCGAACATTTGGTCAGGGAACAAAAGTGGAAATCHAA | |

[symbol]

indicates data missing or illegible when filed

### Fc Region or Fragment Thereof

Here, the above-described first Fc domain and the second Fc domain may each be an Fc region of an immunoglobulin. The Fc region of an immunoglobulin may be an Fc domain variant as well as a wild type Fc domain. Here, the Fc region may be an Fc region of IgG, IgA, IgE, IgD), or IgM.

As used herein, the term “Fc domain variant” may refer to a form which is different from the wild type Fc domain in terms of glycosylation pattern, has a high level of specific glycan species as compared with the wild type Fc domain, a low level of specific glycan species as compared with the wild type Fc domain, or a deglycosylated form. In addition, an aglycosylated Fc domain is included therein. The Fc domain or a variant thereof may be adapted to have an adjusted number of sialic acids, fucosylations, or other types of glycosylations, through modulation of culture conditions or genetic manipulation of a host cell.

In addition, glycosylation of the Fc domain of an immunoglobulin may be modified by conventional methods such as chemical methods, enzymatic methods, and genetic engineering methods using microorganisms. In addition, the Fc domain variant may be in a mixed form of respective Fc regions of immunoglobulin IgG, IgA, IgE, IgD, or IgM. In addition, the Fc domain variant may be a form in which some amino acids of the Fc domain are substituted with other amino acids.

An “amino acid” introduced by the substitution and/or addition may be any one selected from the group consisting of lysine (K), alanine (A), arginine (R), asparagine (N), aspartic acid (D), cysteine (C), glutamine (Q), glutamic acid (E), glycine (G), histidine (H), isoleucine (I), leucine (L), methionine (M), phenylalanine (F), proline (P), serine (S), threonine (T), tryptophan (W), tyrosine (Y), and valine (V).

In one embodiment, the variant of the Fc region may be a form in which amino acids 239 and/or 332 of the CH2 region are substituted with other amino acids (see Kabat numbering system). Specifically, S239 may be substituted with an amino acid other than S, and specifically, S239 may be substituted with S239D. In addition, 1332 may be substituted with an amino acid other than I, and specifically, I332 may be substituted with I332E.

In addition, the Fc region may include a variant or structure of a knob, or a variant or structure of a hole.

As used herein, the term “knob-into-hole” refers to an Fc heterodimerization strategy for producing antibodies that specifically bind to different regions, such as bispecific antibodies, multispecific antibodies, or heterodimeric antibodies. Generally, this technique involves introducing a knob mutation at the interface of a first polypeptide (e.g., the first CH3 domain of a first antibody heavy chain) and a corresponding hole mutation at the interface of a second polypeptide (e.g., the second CH3 domain of a second antibody heavy chain), such that a knob may be placed within the hole to promote heterodimer formation and prevent homodimer formation.

The ‘knob’ variant is constructed by replacing small amino acid side chains from the interface of the first polypeptide (e.g., the first CH3 domain of the first antibody heavy chain) with larger side chains (e.g., arginine, phenylalanine, tyrosine, or tryptophan). The complementary ‘hole’ variant of the same or similar size to the knob is created by replacing large amino acid side chains at the interface of the second polypeptide (e.g., the second CH3 domain of the second antibody heavy chain) with smaller side chains (e.g., alanine, serine, valine, or threonine). The knob and hole may be created by altering the nucleic acid encoding the polypeptide, for example, by site-directed mutagenesis, or by peptide synthesis.

Examples of variants of the Fc region that promote the formation of a heterodimer may include those described in WO2014084607A1 and WO2018059502A1, etc. The disclosures of WO2014084607A1 and WO2018059502A1 are incorporated herein by reference. WO2014084607A1 describes, for example, mutations in the CH3 domain that may comprise (a-1) tryptophan (W) substituted at Lys409 of one CH3 domain that interacts with valine (V) substituted at Asp399 and threonine (T) substituted at Phe405 of another CH3 domain; and (a-2) serine (S) substituted at Tyr349 of one CH3 domain that interacts with tryptophan (W) substituted at Glu357 of another CH3 domain, and in addition, may further comprise (b-1) glutamic acid (E) substituted at Lys360 of one CH3 domain that interacts with arginine (R) substituted at Gln347 of another CH3 domain; and (b-2) glutamic acid (E) substituted at Gln347 and glutamic acid substituted at Lys360 of one CH3 domain that interact with arginine (R) substituted at Gln347 of another CH3 domain. Here, the position of the amino acid residue follows the EU index. WO2018059502A1, for example, describes mutations in the Fc domain including one or more mutations selected from a)-e), respectively: a) L351G, L351Y, L351V, L351P, L351D, L351E, L351K, or L351W; b) T366L, T366P, T366W, or T366V; c) D399C, D399N, D399I, D399G, D399R, D399T, or D399A; d) Y407L, Y407A, Y407P, Y407F, Y407T, or Y407H; and e) K409C, K409P, K409S, K409F, K409V, K409Q, or K409R. Here, the position of the amino acid residue follows the EU index.

### Structure of Fusion Protein

The fusion protein may comprise polypeptide chains represented by the following structural formulas (I), (II), (III), and (IV), respectively:

N′—X-(L1)n-A-C′ (I);

N′—Y-(L2)m-B—C′ (II);

N′—C—C′ (III); and

N′-D—C′ (IV)
wherein, in the structural formulas (I), (II), (III), and (IV),
N′ is the N-terminus of each polypeptide,
C′ is the C-terminus of each polypeptide,
   refers to a linkage,
A, B, C, and D are monomeric polypeptide sequences of an Fc domain each comprising the CH2 and CH3 regions of an immunoglobulin, and optionally further comprising CH4 and/or a hinge sequence, wherein
A forms a dimer with one of C or D to form the first Fc domain (b), and
B forms a dimer with the remaining one of C or D to form the second Fc domain (c);
L1 and L2 are each peptide linker,
n and m are each independently 0 or 1,
X comprises a heavy chain variable region or a light chain variable region of an antibody that specifically binds to an antigen;
Y comprises a light chain variable region or a heavy chain variable region of an antibody that specifically binds to an antigen; and
X and Y pair with each other to form the antigen-binding site (a) that specifically binds to an antigen, and
the polypeptides of (I), (II), (III), and (IV) may be assembled into a fusion protein comprising one antigen-binding site and two Fc domains.

Specifically, X is a first polypeptide sequence of the antigen-binding site, which comprises heavy chain CDR1, CDR2, and CDR3 sequences of an antibody that specifically binds to a first antigen, or a heavy chain variable region of an antibody that specifically binds to a first antigen; Y is a second polypeptide sequence of the antigen-binding site, which comprises light chain CDR1, CDR2, and CDR3 sequences of an antibody that specifically binds to a first antigen, or a light chain variable region of an antibody that specifically binds to a first antigen; and X and Y pair with each other to form the antigen-binding site (a) that specifically binds to an antigen.

According to one embodiment, the CH3 region may be mutated to minimize the interaction between A and B, and between C and D and promote the formation of a heterodimeric Fc between A and C, and between B and D. Specifically, the Fc domain monomer comprises a knob variant or a hole variant that promotes the formation of an Fc heterodimer (heterodimeric Fc); or the Fc domain monomer may comprise a variant that promotes the formation of a heterodimer by electrostatic steering mechanism.

According to one embodiment, X in the structural formula (I) may further comprise a heavy chain CH1 region, and/or Y in the structural formula (II) may further comprise a light chain constant region.

In addition, the fusion protein may comprise polypeptide chains represented by the following structural formulas (I′), (II′), (III), and (IV):

N′—VD1-(L3)p-X-(L1)n-A-C′ (I′);

N′—VD2-(L4)q-Y-(L2)m-B—C′ (II′);

N′—C—C′ (III); and

N′-D-C′ (IV)
wherein, in the structural formulas (I′), (II′), (III), and (IV),
N′ is the N-terminus of the polypeptide chain,
C′ is the C-terminus of the polypeptide chain,
   refers to a linkage,
A, B, C, and D are monomeric polypeptide sequences of an Fc domain each comprising the CH2 and CH3 regions of an immunoglobulin, and optionally further comprising CH4 and/or a hinge sequence, wherein A forms a dimer with one of C or D to form the first Fc domain (b), and B forms a dimer with the remaining one of C or D to form the second Fc domain (c);
L1, L2, L3, and L4 are each peptide linker,
n, m, p, and q are each independently 0 or 1,
VD1 consists of a heavy chain or light chain variable region of an antibody that specifically binds to an antigen, or CDR1, CDR2, and CDR3 of an antibody heavy chain or light chain;
VD2 consists of a light chain or heavy chain variable region of an antibody that specifically binds to an antigen, or CDR1, CDR2, and CDR3 of an antibody heavy chain or light chain;
VD1 and VD2 pair with each other to form a second antibody variable region that specifically binds to a second antigen,
X comprises a heavy chain or light chain variable region of an antibody that specifically binds to an antigen, or CDR1, CDR2, and CDR3 of an antibody heavy chain or light chain;
Y comprises a light chain or heavy chain variable region of an antibody that specifically binds to an antigen, or CDR1, CDR2, and CDR3 of an antibody heavy chain or light chain; and
X and Y pair with each other to form a first antibody variable region that specifically binds to a first antigen, and
VD1-(L3)p-X forms a first polypeptide sequence of the antigen-binding site (a), and VD2-(L4)q-Y forms a second polypeptide sequence of the antigen-binding site (a).

According to one embodiment, the CH3 region may be mutated to minimize the interaction between A and B, and between C and D and promote the formation of a heterodimeric Fc between A and C, and between B and D. Specifically, the Fc domain monomer comprises a knob variant or a hole variant that promotes the formation of an Fc heterodimer (heterodimeric Fc); or the Fc domain monomer may comprise a variant that promotes the formation of a heterodimer by electrostatic steering mechanism.

According to one embodiment, the heavy chain variable region may further comprise a heavy chain CH1 region. In addition, the light chain variable region may further comprise a light chain constant region.

In the structures of the fusion proteins described herein, the binding between X and Y may be achieved i) through a disulfide bond formed by Cys present in CH1 and a light chain constant region, ii) through a disulfide bond formed by Cys present in a heavy chain variable region and a light chain variable region, or iii) through a disulfide bond formed by Cys present in CH1 and a light chain constant region, and a disulfide bond formed by Cys present in a heavy chain variable region and a light chain variable region.

Specifically, the binding between X and Y may be formed by a disulfide bond present between CH₁233 and CL214 based on Kabat numbering system. In addition, X and Y may further comprise Cys through amino acid substitution. Examples of such variants may include mutations in the variable region, and specifically may include mutations at 105C of VH and 43C of VL, or mutations at 44C of VH and 100C of VL based on Kabat numbering system. In one embodiment, the mutation may be Q105C of VH and A43C of VL. In addition, in one embodiment, the mutation may be G44C of VH and Q100C of VL. In addition, examples of variants in the constant region may include mutations at 122C of CH1 and 121C of CL based on Kabat numbering system. In one embodiment, the mutation may be F122C of CH1 and S121C of CL.

### Linker and Hinge

The hinge is a hinge region derived from immunoglobulins. In one embodiment, the antibody hinge region is an IgG hinge region. The IgG hinge region provided herein may be selected, for example, from antibody hinge regions of various IgG subtypes. The table below lists exemplary IgG subtypes with core hinge sequences that may be included in the flexible peptide regions provided herein. In addition, at least one Cys may exist within the hinge. Specifically, 1, 2, or 3 Cys may exist within the hinge.

TABLE 5

| IgG | | SEQ |
|---|---|---|
| subtype | Sequence of core hinge | ID NO |
| IgG1 | EPKSCDKTHTCPPCP | 828 |
| IgG2 | ERKCCVECPPCP | 829 |
| IgG3 | ELKTPLDTTHTCPRCP(EPKSCDTPPPCPRCP)3 | 830 |
| IgG4 | ESKYGPPCPSCP | 831 |

The hinge may be modified to delete disulfide bonds or introduce additional disulfide bonds.

In addition, the linkers L1 and L2 may each comprise 1 to about 70 amino acids. According to one exemplary embodiment, L1 and L2 may each comprise about 5 to about 60 amino acids, about 10 to about 50 amino acids, about 15 to about 40 amino acids, or about 20 to about 30 amino acids. According to another exemplary embodiment, for example, L1 and L2 may each be a peptide consisting of 1-70 amino acid residues, 2-60 amino acid residues, 2-50 amino acid residues, 2-40 amino acid residues, 2-30 amino acid residues, 3-50 amino acid residues, 3-40 amino acid residues, 3-30 amino acid residues, 2-28 amino acid residues, 2-26 amino acid residues, 2-24 amino acid residues, 2-22 amino acid residues, 2-20 amino acid residues, 2-18 amino acid residues, 2-16 amino acid residues, 2-14 amino acid residues, 2-12 amino acid residues, or 2-10 amino acid residues. Specifically, L1 and L2 may include the amino acid sequence of (G4S)o (where o is an integer of 1 to 5) in Table 6 below, but are not limited thereto. In addition, L1 and L2 may have different amino acid sequences. In addition, here, L1 and L2 may comprise at least one Cys. In addition, a disulfide bond may be formed through Cys present in L1 and L2.

TABLE 6

| Sequence of linker | SEQ ID NO |
|---|---|
| GGGGS | 832 |
| GGGGSGGGGS | 833 |
| GGGGSGGGGSGGGGS | 6 |

In addition, the linkers L3 and L4 may each comprise 1 to about 30 amino acids. According to one exemplary embodiment, L3 and L4 may each comprise about 5 to about 25 amino acids, about 10 to about 20 amino acids, or about 15 amino acids. According to another exemplary embodiment, L3 and L4 may each be a peptide consisting of 2-30 amino acid residues, 2-25 amino acid residues, 2-20 amino acid residues, 2-15 amino acid residues, 3-30 amino acid residues, 2-28 amino acid residues, 2-26 amino acid residues, 2-24 amino acid residues, 2-22 amino acid residues, 2-20 amino acid residues, 2-18 amino acid residues, 2-16 amino acid residues, 2-14 amino acid residues, 2-12 amino acid residues, or 2-10 amino acid residues. Specifically, L3 and L4 may include the amino acid sequence of (G4S)o (where o is an integer of 1 to 5) in Table 6 above, but are not limited thereto. In addition, L3 and L4 may have different amino acid sequences.

### Specific Examples of Fusion Proteins

### Fusion Protein Comprising One Antigen-Binding Site and Two Fcs

i) Fusion Protein in which Antigen-Binding Site is Fab

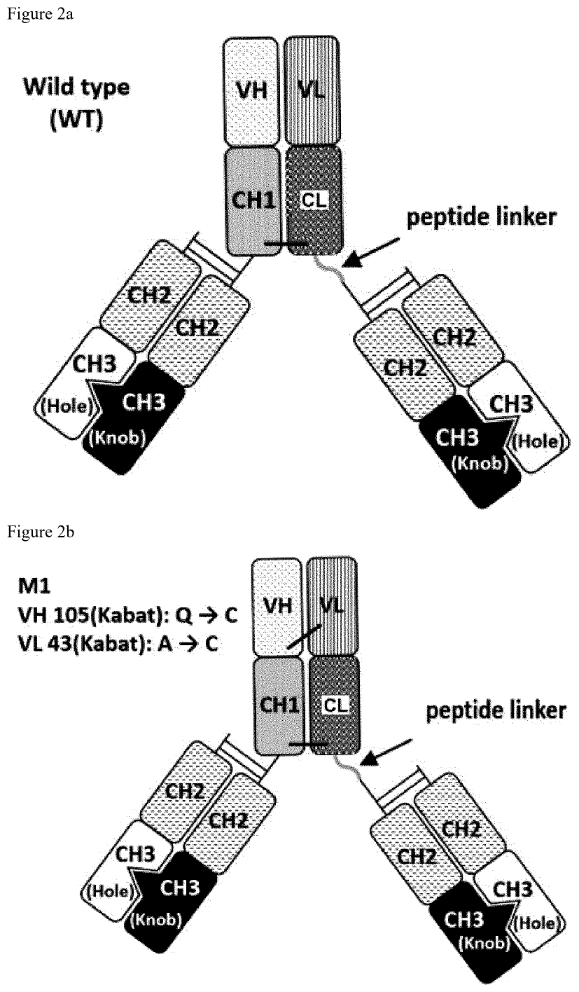

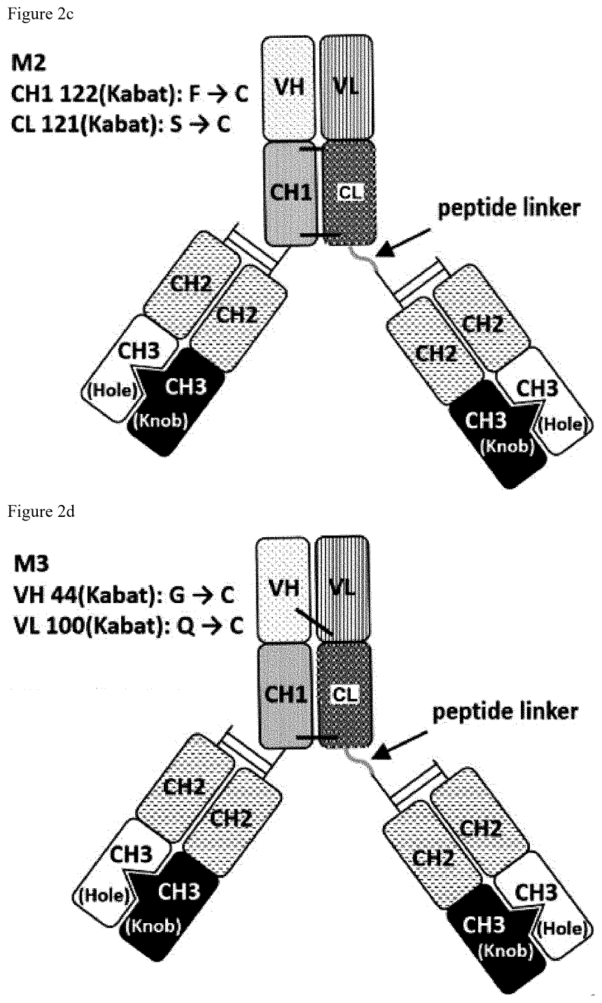

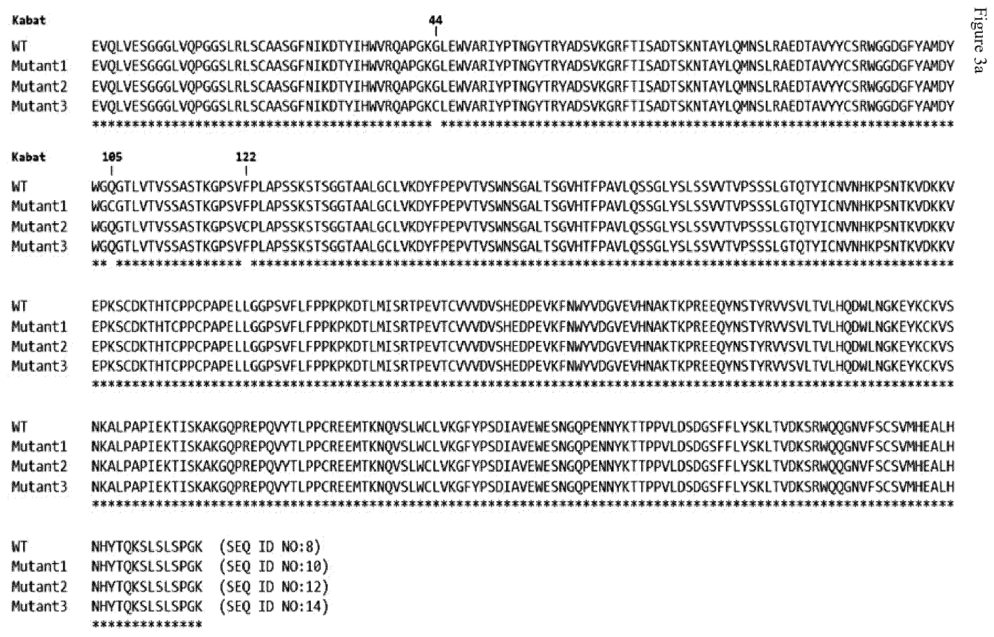

As shown in FIG. 2a, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, the antigen-binding site, antigen, hinge, linker, and Fc are as described above.

As shown in FIG. 2b, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, X comprises the mutation of 105C, and Y comprises the mutation of 43C, and a disulfide bond between Cys is formed. In addition, the antigen-binding site, antigen, hinge, linker, and Fc are as described above.

As shown in FIG. 2c, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, CH1 comprises the mutation of 122C, and the light chain constant region comprises the mutation of 121C, and a disulfide bond between Cys is formed. In addition, the antigen-binding site, antigen, hinge, linker, and Fc are as described above.

As shown in FIG. 2d, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, X comprises the mutation of 44C, and Y comprises the mutation of 100C, and a disulfide bond between Cys is formed. In addition, the antigen-binding site, antigen, hinge, linker, and Fc are as described above.

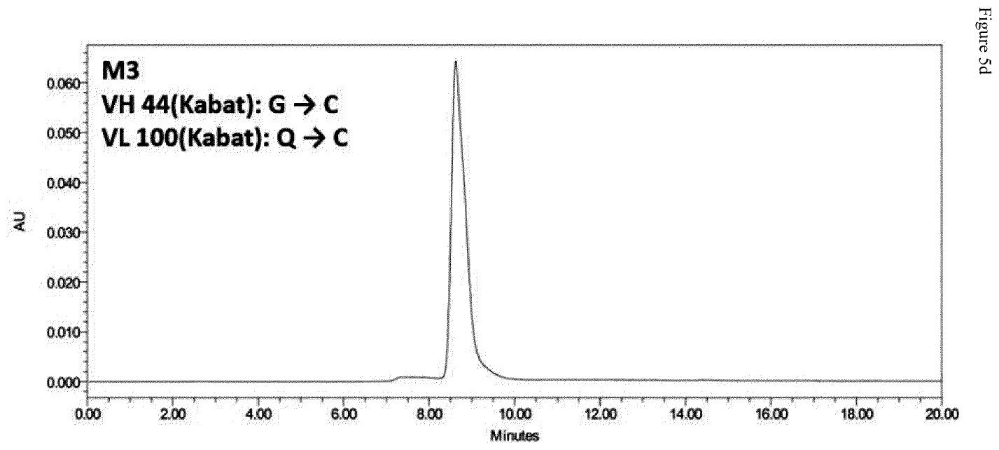

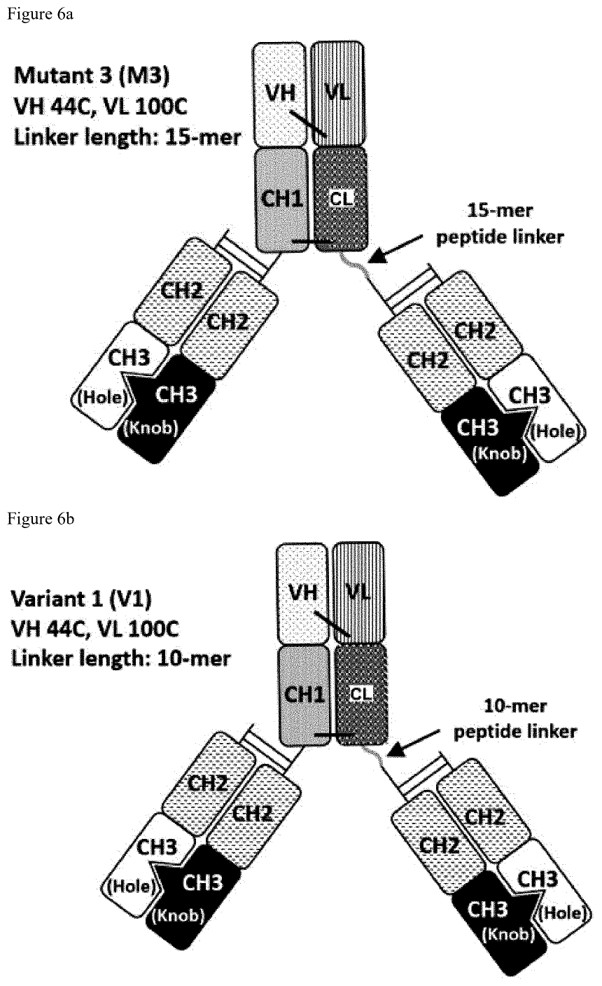

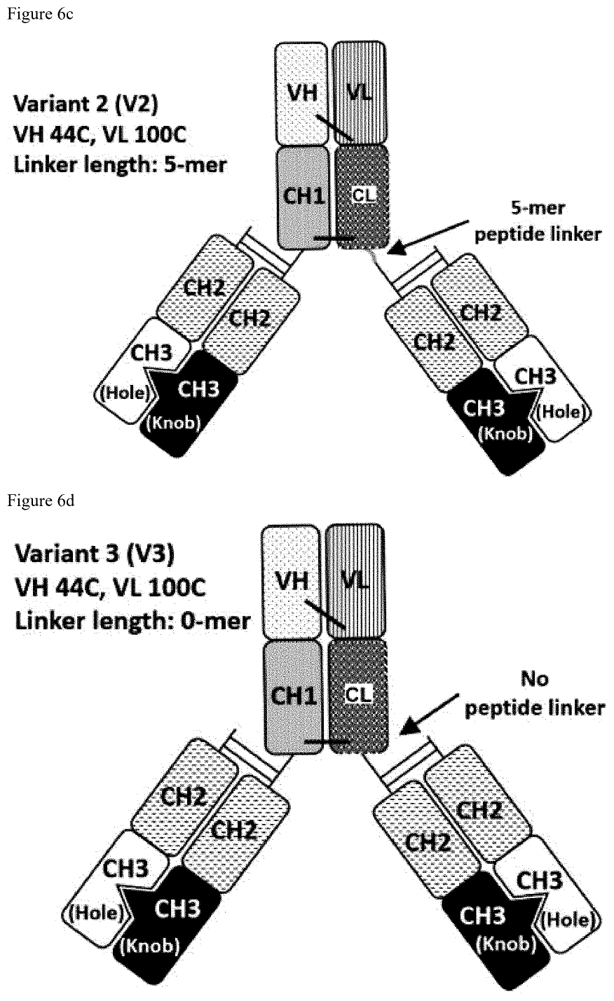

As shown in FIGS. 6a to 6d, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, and CH1 is directly linked to the hinge. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, X comprises the mutation of 44C, and Y comprises the mutation of 100C, and a disulfide bond between Cys is formed. Here, m is 0, and the light chain variable region may be directly linked to the hinge (FIG. 6d). In addition, m is 1, and L2 may include a 15-mer peptide linker (FIG. 6a), a 10-mer peptide linker (FIG. 6b), or a 5-mer peptide linker (FIG. 6c). In addition, the antigen-binding site, antigen, hinge, linker, and Fc are as described above.

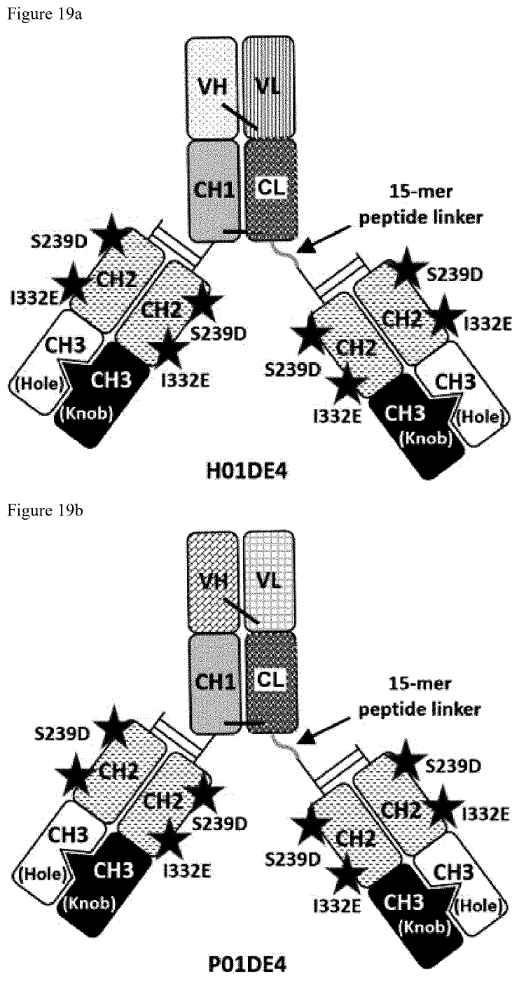

As shown in FIG. 19a, the fusion protein comprises polypeptides of the structural formulas (I), (II), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, X comprises the mutation of 44C, and Y comprises the mutation of 100C, and a disulfide bond between Cys is formed. Here, all CH2s of A, B, C, and D comprise the 239D and 332E mutations. In addition, the antigen-binding site, antigen, hinge, linker, and Fc are as described above.

ii) Fusion Protein in which Antigen-Binding Site is Fv

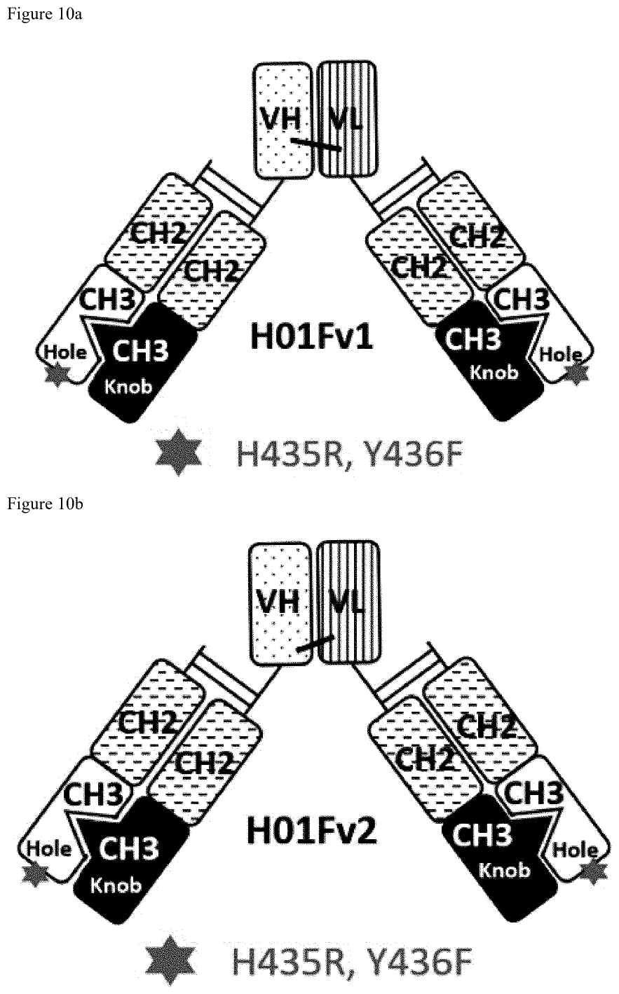

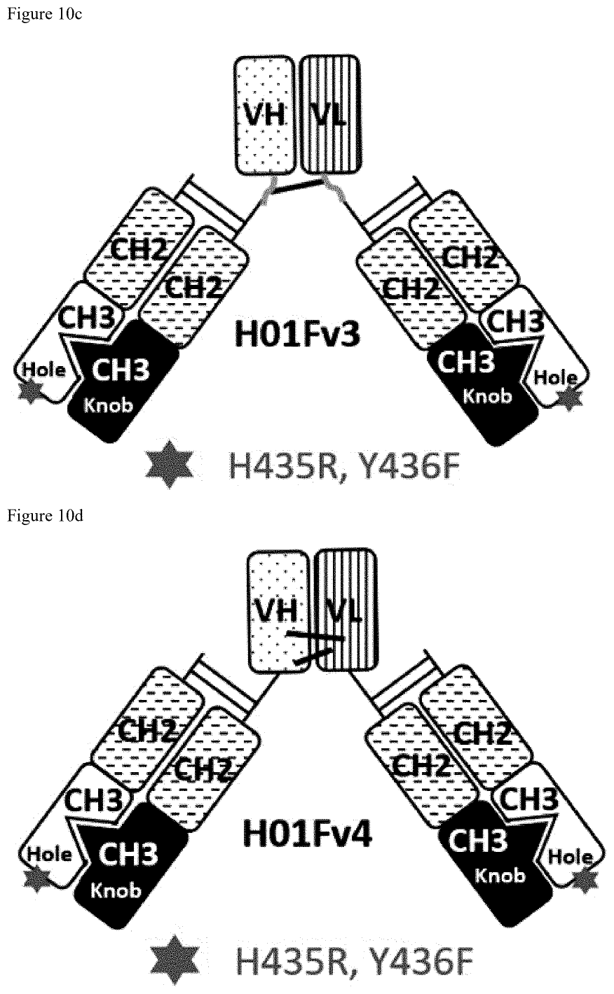

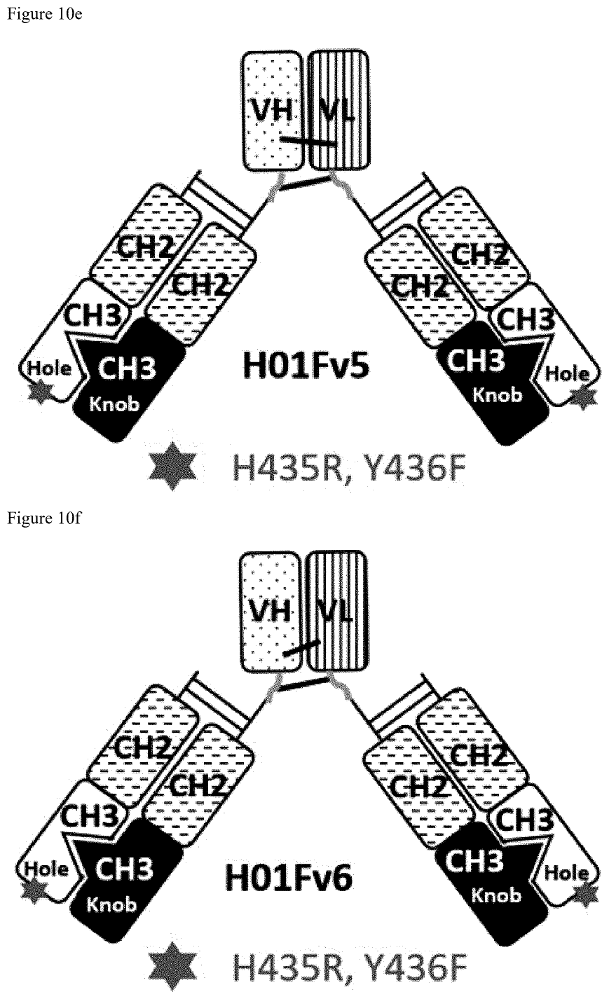

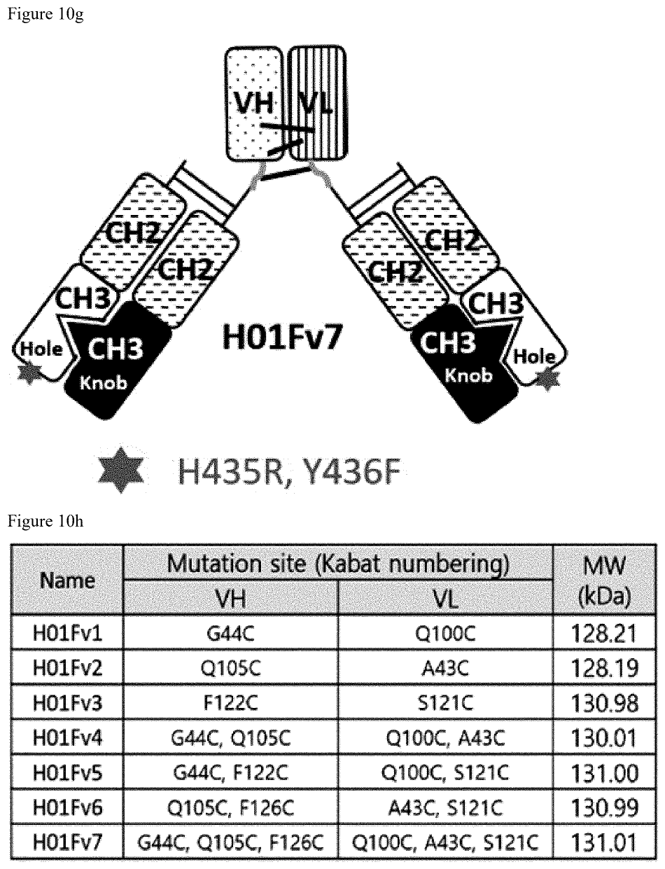

As shown in FIG. 10, the fusion protein comprises polypeptides of the structural formulas (I), (II), (III), and (IV), where X and Y are attached to each other by at least one Cys present therein to form a Fv structure. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. X may comprise the mutations of 44C, 105C, 122C, 44C/105C, 44C/122C, 105C/126C or 44C/105C/126C, and Y may comprise the mutations of 100C, 43C, 121C, 100C/43C, 100C/121C, 43C/121C, or 100C/43C/121C. In addition, a disulfide bond may be formed by Cys present in L1 and L2. In addition, the antigen-binding site, antigen, hinge, linker, and Fc are as described above.

### Fusion Protein Comprising Two Antigen-Binding Sites and Two Fcs

iii) Fusion Protein in which Antigen-Binding Site is Fab

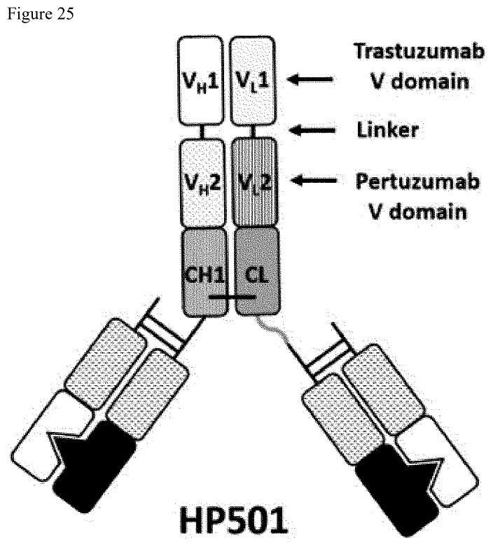

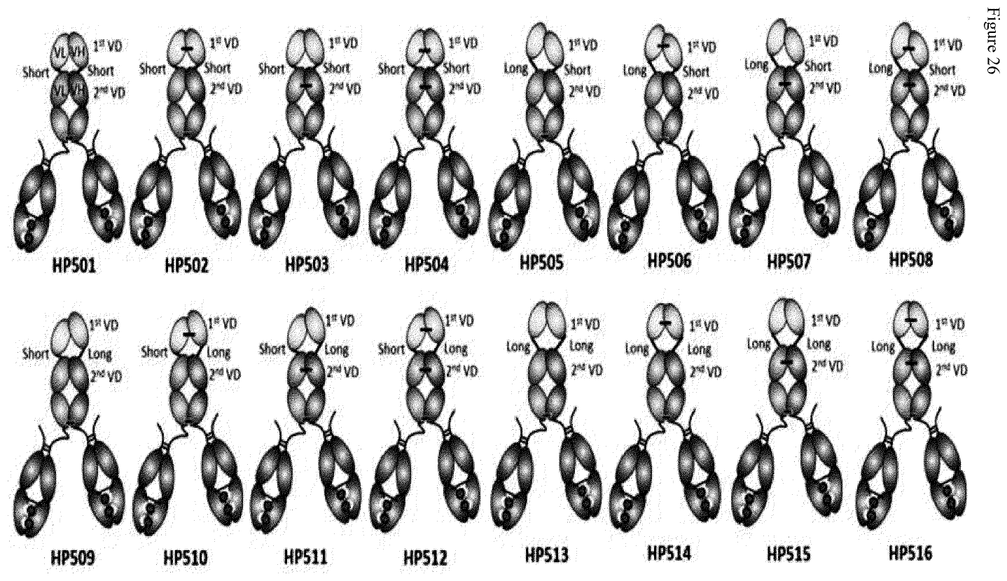

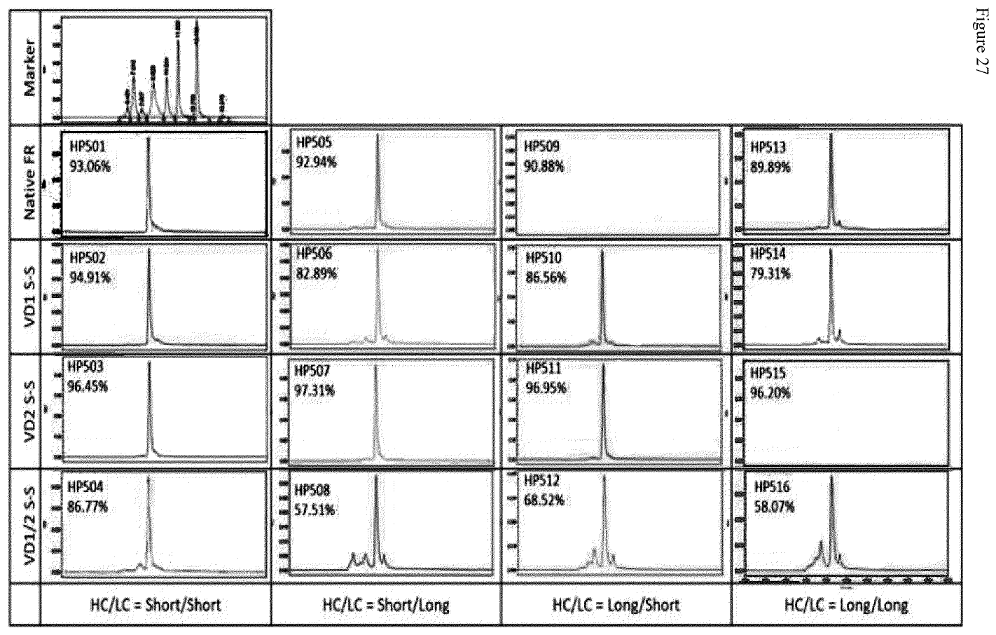

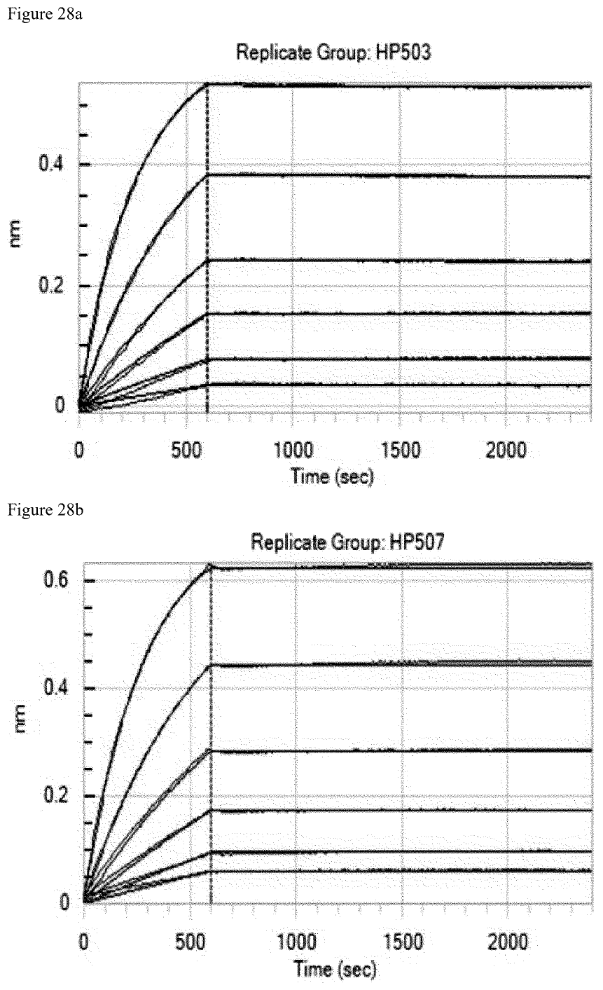

As shown in FIG. 25, the fusion protein comprises polypeptides of the structural formulas (I′), (II′), (III), and (IV), where X is a heavy chain variable region and further comprises CH1, and Y is a light chain variable region and comprises a light chain constant region. In addition, they are attached to each other by Cys in the CH1 structure and the light chain variable region to form a Fab structure. Here, n is 0, CH1 is directly linked to the hinge, m is 1, and L2 includes a peptide linker. Here, A and C are attached to each other to form the first Fc domain, and B and D are attached to each other to form the second Fc domain. In addition, the CH3 region of A comprises a hole variant, and the CH3 region of C comprises a knob variant. In addition, the CH3 region of B comprises a hole variant, and the CH3 region of D comprises a knob variant. In addition, VD1 in the structural formula (I′) is a heavy chain variable region, and VD2 in the structural formula (II′) is a light chain variable region, and VD1 and VD2 pair with each other to form Fv. In addition, p and q are each 1, and L3 and L4 are peptide linkers. As a non-limiting example, X and Y may pair with each other to form the variable region of pertuzumab, and VD1 and VD2 may pair with each other to form the variable region of trastuzumab. In addition, the antigen-binding site, antigen, hinge, linker, and Fc are as described above.

As shown in FIG. 26, the peptide linkers of L3 and L4 in the structural formulas (I′) and (II′) may be of various lengths. In addition, the first antigen-binding site formed by pairing between X and Y, and the second antigen-binding site formed by pairing between VD1 and VD2 may be the same or different. In addition, L1 and L2 may also comprise various peptide linkers.

### Use of Fusion Protein

In another aspect of the present invention, there is provided a pharmaceutical composition for preventing or treating cancer, comprising the fusion protein as an active ingredient.

Here, cancer may be any one selected from the group consisting of gastric cancer, liver cancer, lung cancer, large intestine cancer, breast cancer, prostate cancer, skin cancer, bone cancer, multiple myeloma, glioma, ovarian cancer, pancreatic cancer, cervical cancer, thyroid cancer, laryngeal cancer, acute myeloid leukemia, chronic myeloid leukemia, acute lymphoblastic leukemia, chronic lymphoblastic leukemia, brain tumor, neuroblastoma, retinoblastoma, head and neck cancer, salivary gland cancer, and lymphoma.

### Polynucleotide Encoding Fusion Protein

In another aspect of the present invention, there is provided a polynucleotide encoding a polypeptide of the structural formula (I), (II), (III), and/or (IV).

In another aspect of the present invention, there is provided a polynucleotide encoding a polypeptide of the structural formula (I′), (II′), (III), and/or (IV).

The polynucleotide may further comprise a nucleic acid encoding a signal sequence or a leader sequence. As used herein, the term “signal sequence” refers to a signal peptide that directs secretion of a target protein. The signal peptide is translated and then cleaved in a host cell. Specifically, the signal sequence is an amino acid sequence that initiates migration of a protein across the endoplasmic reticulum (ER) membrane.

The signal sequences are well known in the art for their characteristics. Such signal sequences typically comprise 16 to 30 amino acid residues, and may comprise more or fewer amino acid residues than such amino acid residues. A typical signal peptide consists of three regions, that is, a N-terminal region, a central hydrophobic region, and a more polar C-terminal region. The central hydrophobic region comprises 4 to 12 hydrophobic residues that cause the signal sequence to be immobilized during migration of an immature polypeptide through the membrane lipid bilayer.

After initiation, signal sequences are cleaved in the lumen of ER by cellular enzymes, commonly known as signal peptidases. Here, the signal sequence may be a secretory signal sequence of tPa (tissue plasminogen activator), HSV gDs (signal sequence of Herpes simplex virus glycoprotein D), or a growth hormone. Preferably, a secretory signal sequence used in higher eukaryotic cells including mammals and the like may be used. In addition, as the signal sequence, a wild type signal sequence may be used, or a signal sequence that has been substituted with a codon having high expression frequency in a host cell may be used.

Vector Loaded with Polynucleotide

In another aspect of the present invention, there is provided a vector comprising the polynucleotide. The vector may comprise a polynucleotide encoding a polypeptide of the structural formula (I), (II), (III), and/or (IV). In addition, the vector may comprise a polynucleotide encoding a polypeptide of the structural formula (I′), (II′), (III), and/or (IV).

The vector may be introduced into a host cell to be recombined with and inserted into the genome of the host cell. Alternatively, the vector is understood as nucleic acid means comprising a polynucleotide sequence which is autonomously replicable as an episome. The vectors include linear nucleic acids, plasmids, phagemids, cosmids, RNA vectors, viral vectors, and analogs thereof. Examples of the viral vector include, but are not limited to, retroviruses, adenoviruses, and adeno-associated viruses.

Specifically, the vector may include plasmid DNA, phage DNA, and the like; and commercially developed plasmids (pUC18, pBAD, pIDTSAMRT-AMP, and the like), E. coli-derived plasmids (pYG601BR322, pBR325, pUC118, pUC119, and the like), Bacillus subtilis-derived plasmids (pUB110, pTP5, and the like), yeast-derived plasmids (YEp13, YEp24, YCp50, and the like), phage DNA (Charon4A, Charon21A, EMBL3, EMBL4, λgt10, λgt11, λZAP, and the like), animal viral vectors (retroviruses, adenoviruses, vaccinia viruses, and the like), insect viral vectors (baculoviruses and the like). Since the vector exhibits different expression levels and modification of a protein depending on a host cell, it is preferred to select and use a host cell which is most suitable for the purpose.

As used herein, the term “gene expression” or “expression” of a target protein is understood to mean transcription of DNA sequences, translation of mRNA transcripts, and secretion of fusion protein products or fragments thereof. A useful expression vector may be RcCMV (Invitrogen, Carlsbad) or a variant thereof. The expression vector may comprise human cytomegalovirus (CMV) promoter for promoting continuous transcription of a target gene in mammalian cells, and a bovine growth hormone polyadenylation signal sequence for increasing the stability level of RNA after transcription.

### Transformed Cell Expressing Fusion Protein

In another aspect of the present invention, there is provided a transformed cell expressing. Specifically, the transformed cell may be one into which the vector has been introduced.

Host cells for the transformed cell may include, but are not limited to, prokaryotic cells, eukaryotic cells, and cells of mammalian, plant, insect, fungal, or cellular origin. As an example of the prokaryotic cells, E. coli may be used. In addition, as an example of the eukaryotic cells, yeast may be used. In addition, for the mammalian cells, CHO cells, F2N cells, CSO cells, BHK cells, Bowes melanoma cells, HeLa cells, 911 cells, AT1080 cells, A549 cells, HEK 293 cells, HEK293T cells, or the like may be used. However, the mammalian cells are not limited thereto, and any cells which are known to those of ordinary skill in the art to be usable as mammalian host cells may be used.

In addition, for the introduction of an expression vector into the host cell, CaCl₂) precipitation, Hanahan method whose efficiency has been increased efficiency by using a reducing agent such as dimethyl sulfoxide (DMSO) in CaCl₂) precipitation, electroporation, calcium phosphate precipitation, protoplast fusion, agitation using silicon carbide fiber, Agrobacteria-mediated transformation, transformation using PEG, dextran sulfate-, Lipofectamine-, and dry/inhibition-mediated transformation, and the like may be used.

As described above, for optimization of properties of a fusion protein as a therapeutic agent or for any other purpose, glycosylation pattern of the fusion protein (for example, sialic acids, fucosylations, glycosylations) may be adjusted by manipulating, through methods known to those of ordinary skill in the art, glycosylation-related genes possessed by host cells.

### Method for Producing a Fusion Protein

In another aspect of the present invention, there is provided a method for producing a fusion protein comprising an antigen-binding site, a first Fc domain or a variant thereof, and a second Fc domain or a variant thereof, the method comprising the steps of: i) culturing the transformed cells; and ii) collecting the produced fusion proteins.

The step of culturing the transformed cells may be carried out using methods well known in the art. Specifically, the culture may be carried out in a batch process, or carried out continuously in a fed batch or repeated fed batch process.

### Composition or Preparation Containing Fusion Protein

In another aspect of the present invention, there is provided a pharmaceutical composition comprising the fusion protein as an active ingredient.

The pharmaceutical composition may be used for the prevention or treatment of cancer, such as any one cancer selected from the group consisting of gastric cancer, liver cancer, lung cancer, large intestine cancer, breast cancer, prostate cancer, gallbladder cancer, bladder cancer, kidney cancer, esophageal cancer, skin cancer, rectal cancer, osteosarcoma, multiple myeloma, glioma, ovarian cancer, pancreatic cancer, cervical cancer, endometrial cancer, thyroid cancer, laryngeal cancer, testicular cancer, mesothelioma, acute myeloid leukemia, chronic myeloid leukemia, acute lymphoblastic leukemia, chronic lymphoblastic leukemia, brain tumor, neuroblastoma, retinoblastoma, head and neck cancer, salivary gland cancer, and lymphoma.

A preferred dosage of the pharmaceutical composition varies depending on the patient's condition and body weight, severity of disease, form of drug, route and duration of administration and may be appropriately selected by those of ordinary skill in the art. In the pharmaceutical composition for treating or preventing tumor of the present invention, the active ingredient may be contained in any amount (effective amount) depending on application, dosage form, blending purpose, and the like, as long as the active ingredient may exhibit therapeutic activity against tumor or, in particular, may exhibit a therapeutic effect on cancer. A conventional effective amount thereof will be determined within a range of 0.001% to 20.0% by weight, based on the total weight of the composition. Here, the term “effective amount” refers to an amount of an active ingredient that may induce an effect of improving or treating the condition of a disease, especially an effect of improving or treating the condition of cancer. Such an effective amount may be experimentally determined within the scope of common knowledge of those of ordinary skill in the art.

As used herein, the term “treatment” may be used to mean both therapeutic and prophylactic treatment. Here, prophylaxis may be used to mean that a pathological condition or disease of a subject is alleviated or mitigated. In one embodiment, the term “treatment” includes both application or any form of administration for treating a disease in a mammal, including a human. In addition, the term includes inhibiting or slowing down the progression of a disease; and includes meanings of restoring or repairing impaired or lost function so that a disease is partially or completely alleviated; stimulating inefficient processes; or alleviating a serious disease.

Pharmacokinetic parameters such as bioavailability and underlying parameters such as clearance rate may also affect efficacy. Therefore, “improved efficacy” (for example, improvement in efficacy) may be due to improved pharmacokinetic parameters and improved efficacy, which may be measured by comparing clearance rate in test animals or human subjects, and parameters such as tumor treatment or improvement.

As used herein, the term “therapeutically effective amount” or “pharmaceutically effective amount” refers to an amount of a compound or composition effective to prevent or treat the disease in question, which is sufficient to treat the disease at a reasonable benefit/risk ratio applicable to medical treatment and does not cause adverse effects. A level of the effective amount may be determined depending on factors including the patient's health condition, type and severity of disease, activity of drug, the patient's sensitivity to drug, mode of administration, time of administration, route of administration and excretion rate, duration of treatment, combined or simultaneously used drugs, and other factors well known in the medical field. In one embodiment, the therapeutically effective amount means an amount of drug effective to treat cancer.

Here, the pharmaceutical composition may further comprise a pharmaceutically acceptable carrier. The pharmaceutically acceptable carrier may be any carrier as long as the carrier is a non-toxic substance suitable for delivery to a patient. Distilled water, alcohol, fat, wax, and inert solid may be contained as the carrier. A pharmaceutically acceptable adjuvant (buffer, dispersant) may also be contained in the pharmaceutical composition.

Specifically, by including a pharmaceutically acceptable carrier in addition to the active ingredient, the pharmaceutical composition may be prepared into a parenteral formulation depending on its route of administration using conventional methods known in the art. Here, the term “pharmaceutically acceptable” means that the carrier does not have more toxicity than the subject to be applied (prescribed) may adapt while not inhibiting activity of the active ingredient.

When the pharmaceutical composition is prepared into a parenteral formulation, it may be made into preparations in the form of injections, transdermal patches, nasal inhalants, or suppositories with suitable carriers according to methods known in the art. In a case of being made into injections, sterile water, ethanol, polyol such as glycerol or propylene glycol, or a mixture thereof may be used as a suitable carrier; and an isotonic solution, such as Ringer's solution, phosphate buffered saline (PBS) containing triethanol amine or sterile water for injection, and 5% dextrose, or the like may preferably be used. Formulation of pharmaceutical compositions is known in the art, and reference may specifically be made to Remington's Pharmaceutical Sciences (19th ed., 1995) and the like. This document is considered part of the present specification.

A preferred dosage of the pharmaceutical composition may range from 0.01 μg/kg to 10 g/kg, or 0.01 mg/kg to 1 g/kg, per day, depending on the patient's condition, body weight, sex, age, severity of the patient, and route of administration. The dosage may be administered once a day or may be divided into several times a day. Such a dosage should not be construed as limiting the scope of the present invention in any aspect.

Subjects to which the pharmaceutical composition may be applied (prescribed) are mammals including dogs, cats, humans, etc., with humans being particularly preferred. In addition to the active ingredient, the pharmaceutical composition of the present invention may further comprise any compound or natural extract, which is known to have a therapeutic effect on tumor.

### Treatment Method Using Fusion Protein

In another aspect of the present invention, there is provided a method for treating or preventing cancer, comprising administering a fusion protein comprising an antigen-binding site, a first Fc domain or a variant thereof, and a second Fc domain or a variant thereof administering to a subject.

In another aspect of the present invention, there is provided a use of a fusion protein comprising an antigen-binding site, a first Fc domain or a variant thereof, and a second Fc domain or a variant thereof for the treatment of cancer.

Here, the subject may be an individual suffering from cancer. In addition, the subject may be a mammal, preferably a human.

Route of administration, dosage, and frequency of administration of the fusion protein may vary depending on the patient's condition and the presence or absence of side effects, and thus the fusion protein may be administered to a subject in various ways and amounts. The optimal administration method, dosage, and frequency of administration may be selected in an appropriate range by those of ordinary skill in the art. In addition, the fusion protein may be administered in combination with other drugs or physiologically active substances whose therapeutic effect is known with respect to a disease to be treated, or may be formulated in the form of combination preparations with other drugs.

### MODE FOR CARRYING OUT THE INVENTION

Hereinafter, the present invention will be described in more detail by way of the following examples. However, the following examples are only for illustrating the present invention, and the scope of the present invention is not limited only to these examples.

### Example 1. Design, Preparation and Analysis of a Novel Antibody with Two Fc Domains

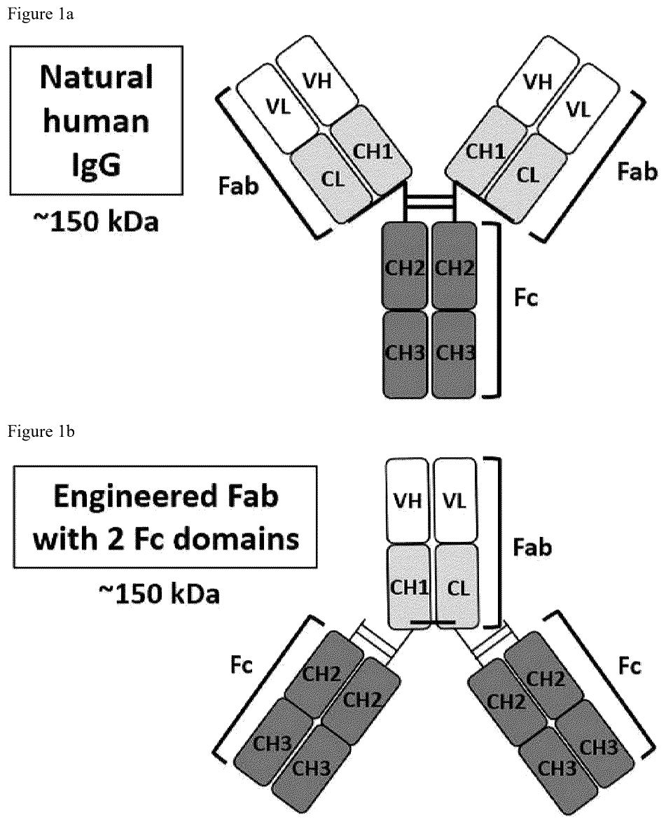

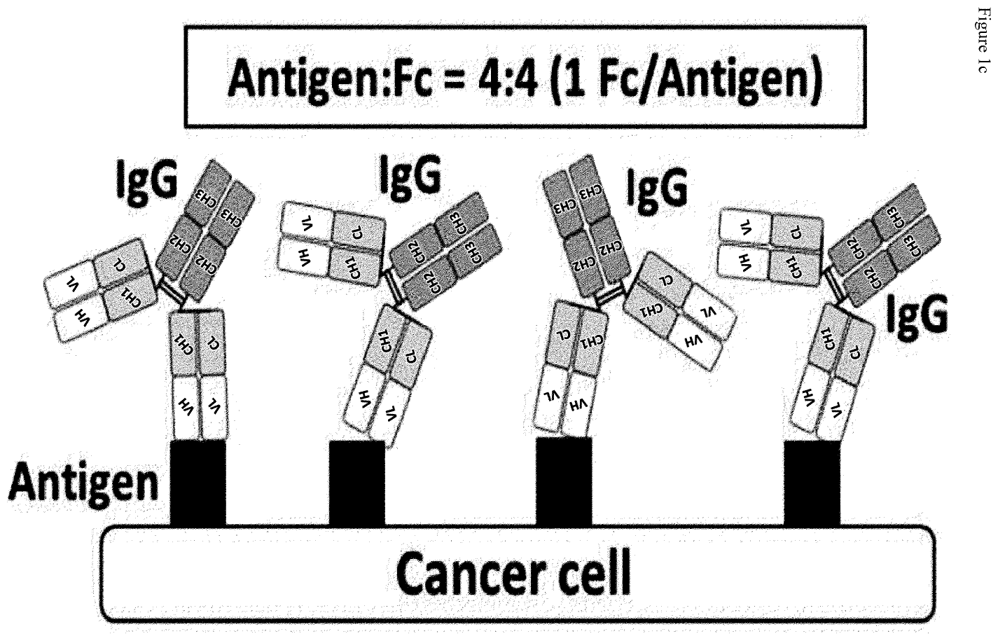

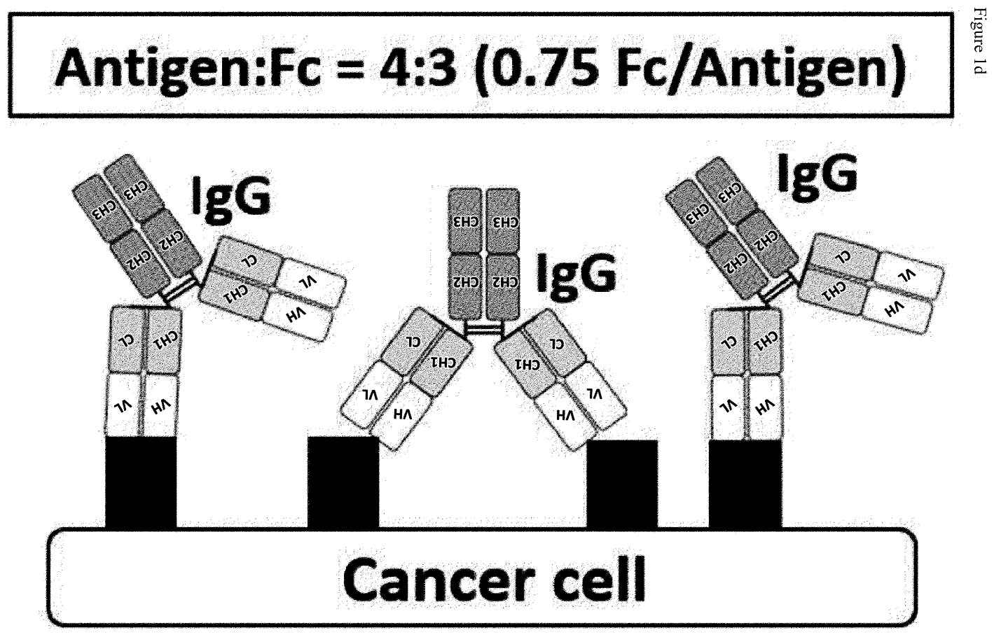

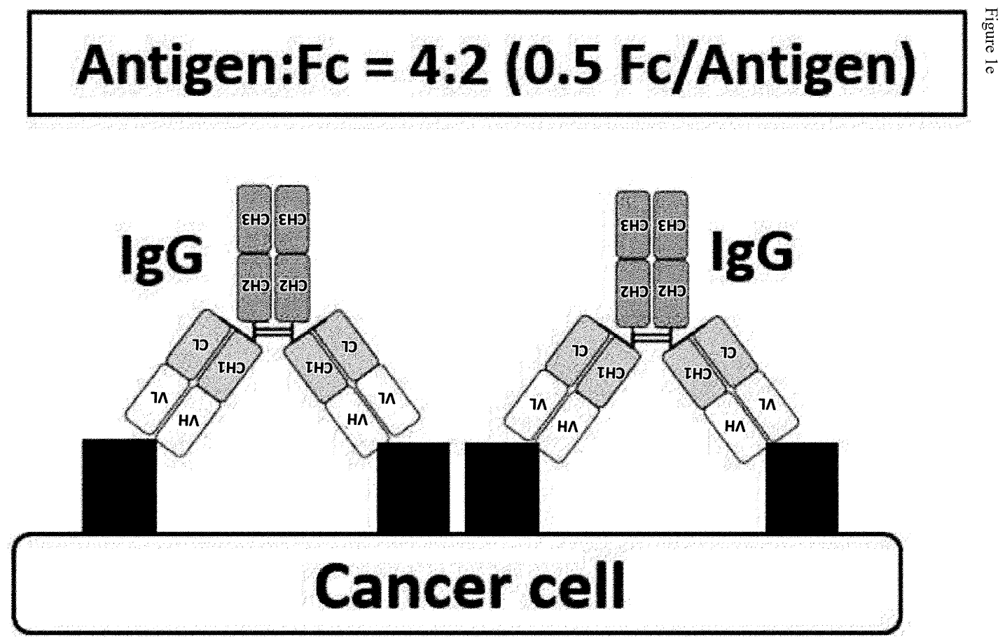

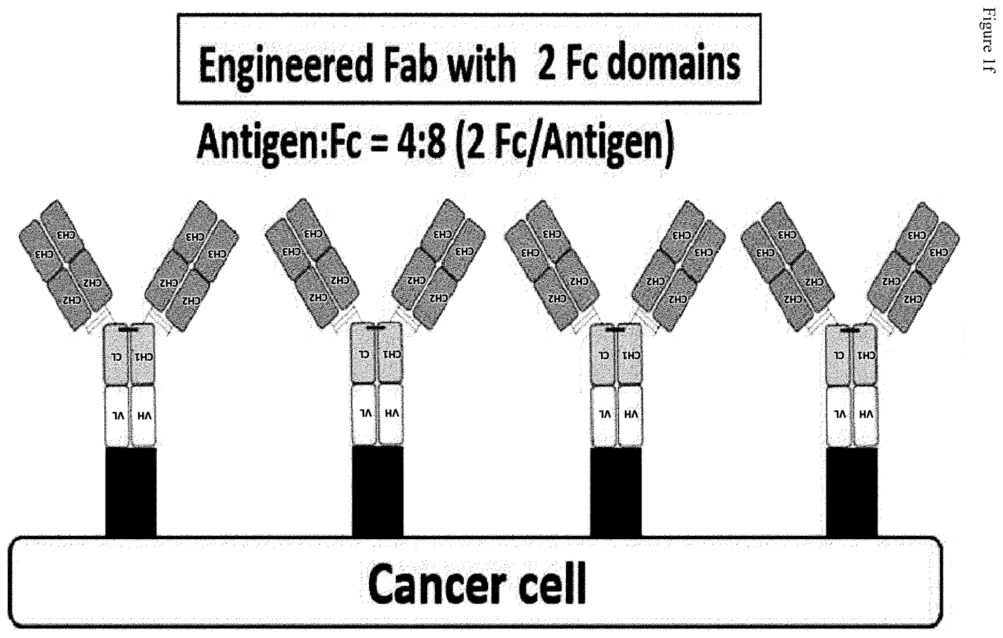

Naturally occurring human immunoglobulin G (IgG) consists of two fragment antigen-binding (Fab) regions and one fragment crystallizable (Fc) region (FIG. 1a). Human IgG binds to the target antigen in a monovalent or bivalent manner, and in some cases has 0.5 to 1 Fc region per target antigen (FIGS. 1c, 1d, and 1e).

The object of the present invention is to improve effector function by increasing the amount of Fc region present per antigen while having a molecular weight similar to that of an antibody and having a homogeneous composition. Therefore, we designed a novel antibody format with two Fc regions that has a molecular weight similar to that of a natural human IgG antibody (approximately 150 kDa) (FIG. 1b). This form binds to a cancer cell surface antigen and enables Fc regions to be present on a cancer cell surface a maximum of four times compared to conventional antibodies (FIGS. 1b and 1f).

In order to implement the novel antibody format mentioned above, trastuzumab was used as a template (FIG. 2a). In order to improve pairing between the VH-CH1 region and the VL-CL region of the trastuzumab Fab region, a specific amino acid was substituted with cysteine to introduce an artificial disulfide bond (FIGS. 2b, 2c, and 2d).

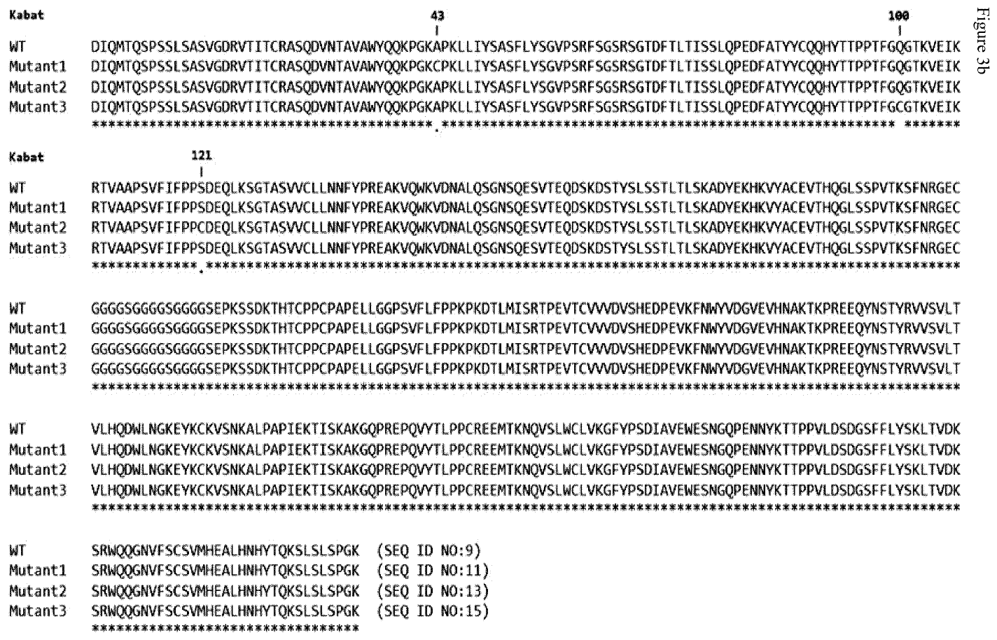

Fab in which glutamine (Q) at number 105 of the heavy chain and alanine (A) at number 43 of the light chain are substituted with cysteines, Fab in which phenylalanine (F) at number 122 of the heavy chain and serine (S) at number 121 of the light chain are substituted with cysteines, and Fab in which glycine (G) at number 44 of the heavy chain and glutamine (Q) at number 100 of the light chain are substituted with cysteines were designed, and they were referred to as Mutant 1, Mutant 2, and Mutant 3, respectively (FIGS. 2b, 2c, 2d, 3a, and 3b). Based on the above, the Fab-(Fc)₂ structure with trastuzumab as a template was referred to as wild type (WT) (FIG. 2a), and the Fab-(Fc)₂ structures with Fab corresponding to Mutants 1 to 3 were referred to as respective M1, M2, and M3 (FIGS. 2b, 2c, 2d, 3a, and 3b).

The notation of the positions in amino acids constituting an antibody follows the Kabat numbering system. In order to minimize unwanted Fc-related byproducts, knob-into-hole mutation technology (Merchant et al., Nat. Biotechnol. 1998) was applied to the Fc domain (SEQ ID NO: 3) of human immunoglobulin G1 (IgG1) to design polypeptides of Fc with knob mutation (S354C and T366W; SEQ ID NO: 4) and Fc with hole mutation (Y349C, T366S, L368A, and Y407V; SEQ ID NO: 5). In order to provide additional flexibility between the CL domain and hinge region, a (G4S)₃ linker was introduced (SEQ ID NO: 6; FIG. 2). The expression of WT was performed by co-transfection of vectors capable of expressing polypeptides corresponding to Fc-Hole (SEQ ID NO: 7), TraH-WT-Knob (SEQ ID NO: 8), and TraL-WT-Knob (SEQ ID NO: 9) into the EXPICHO-S™ (Gibco, A29127) cell line.

M1 consists of Fc-Hole (SEQ ID NO: 7), TraH-Q105C-Knob (SEQ ID NO: 10), and TraL-A43C-Knob (SEQ ID NO: 11), M2 consists of Fc-Hole (SEQ ID NO: 7), TraH-F122C-Knob (SEQ ID NO: 12), and TraL-S121C-Knob (SEQ ID NO: 13), and M3 consists of Fc-Hole (SEQ ID NO: 7), TraH-G44C-Knob (SEQ ID NO: 14), and TraL-Q100C-Knob (SEQ ID NO: 15). All of M1, M2, and M3 were expressed in the EXPICHO-S™ (Gibco, A29127) cell line. They were purified using an AKTA pure 25 (Cytiva) or AKTA avant 150 (Cytiva) protein isolation and purification system equipped with a CAPTURESELECT™ CH1-XL Pre-packed Column (Thermo Scientific, 494346205) purification column, and the purified product was further subjected to affinity chromatography using KappaSelect resin (Cytiva, 17545801), and the sample was concentrated using an Amicon Ultra-15 Centrifugal Filter Unit (Merck millipore). For the final purified product, the absorbance of the sample at 280 nm was measured using a NanoDrop One trace spectrophotometer (Thermo Fisher Scientific), and the concentration was quantified based on the sample's intrinsic extinction coefficient and molecular weight.

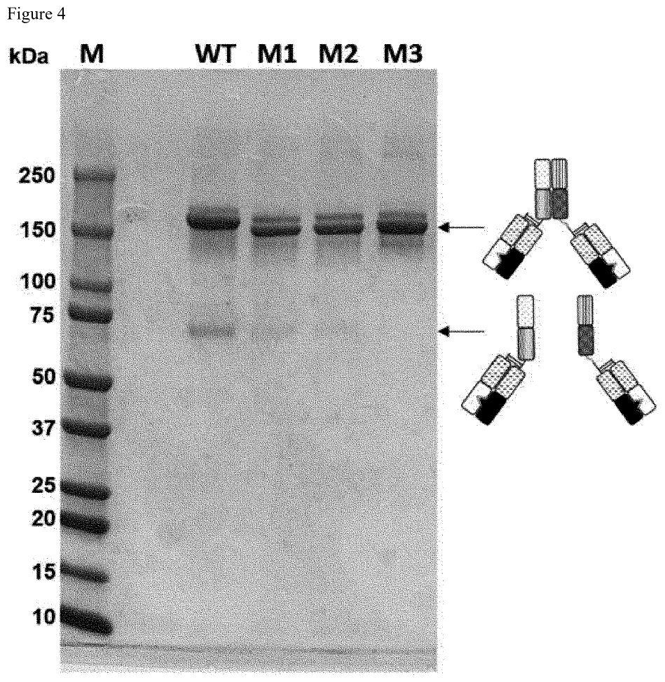

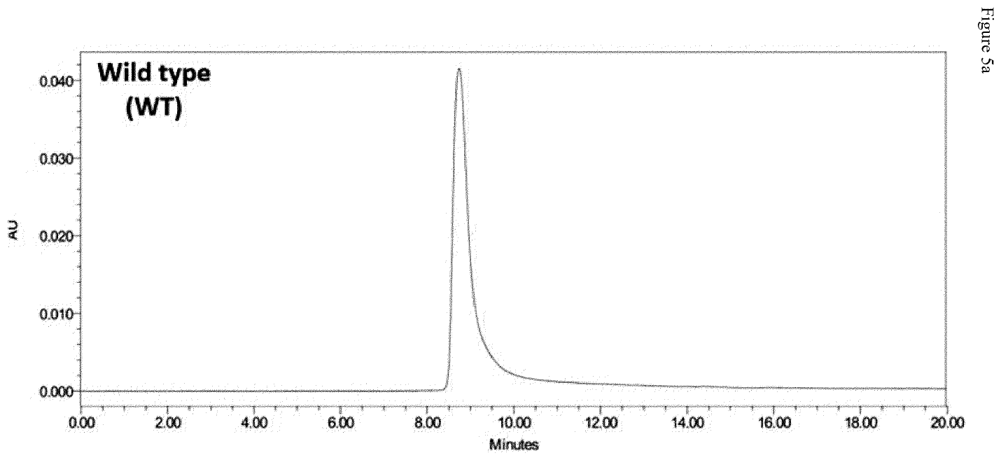

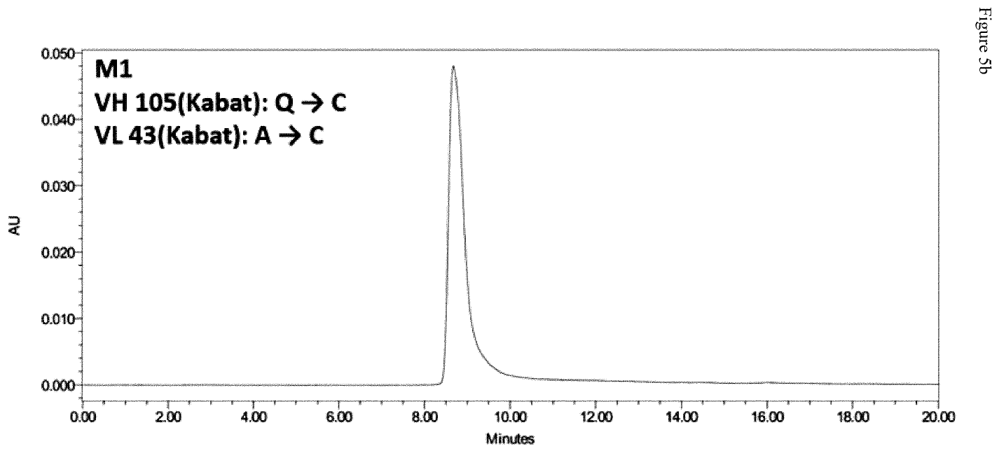

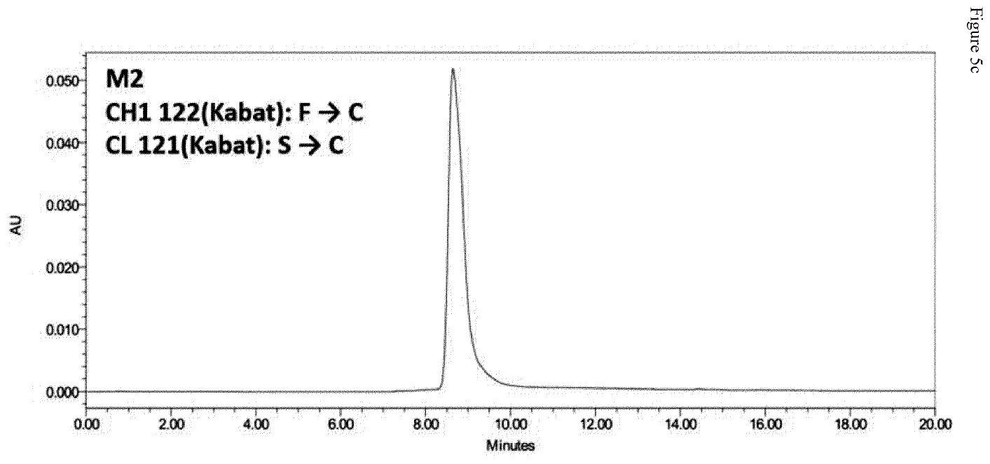

The purified product was analyzed using sodium dodecyl sulfate polyacrylamide gel electrophoresis (SDS-PAGE) and size exclusion chromatography (SEC) (FIGS. 4 and 5a to 5d). Bio-rad's electrophoresis gel and system were used for SDS-PAGE analysis, and samples were analyzed under non-reducing conditions, and size of each band were identified using Coomassie Brilliant Blue staining (FIG. 4). WT, M1, M2, and M3 were identified at about 150 kDa, and monomers were identified at about 75 kDa in WT which has no additional disulfide bond introduced into the Fab interface (FIG. 4). Similarly, for M1 and M2, a trace amount of monomer was identified at 75 kDa. For M3, almost no monomers were identified probably because most of the monomers were easily paired through the formation of disulfide bonds (FIG. 4).

For size exclusion chromatography analysis, an ALLIANCE® HPLC-e2695 Separations Module (Waters, 2695) equipped with an Agilent Bio SEC-3 HPLC column (Agilent, 5190-2511) was used. The analysis showed that the main product was identified at a retention time of 8.6 to 8.8 minutes (FIGS. 5a, 5b, 5c, and 5d).

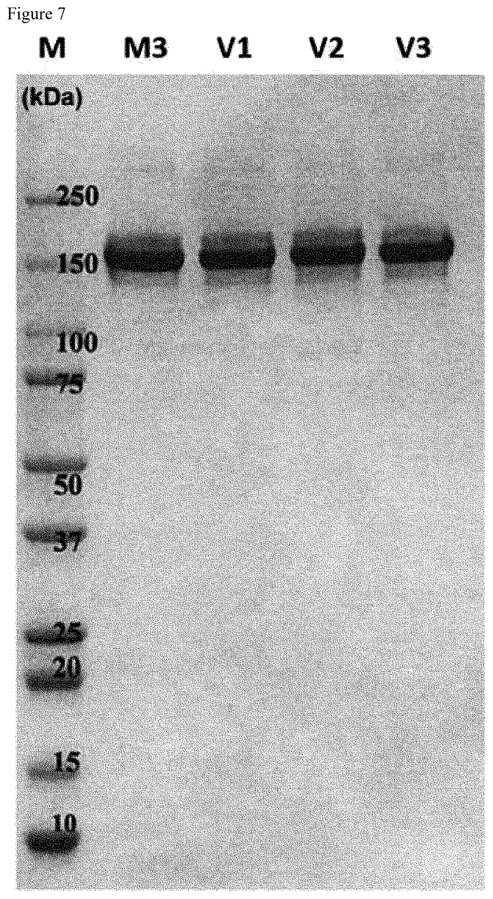

Based on the M3 structure, the impact of the linker that connects the CL domain with the hinge region on the structural integrity of the antibody were analyzed. M3 has a 15-mer polypeptide linker consisting of (G₄S)₃, and V1 (SEQ ID NO: 7, 14, and 16) and V2 (SEQ ID NO: 7, 14, and 17) have polypeptide linkers of (G4S)₂ and G4S, respectively, and V3 (SEQ ID NO: 7, 14, and 18) directly linked the CL domain and the hinge region without a linker (FIG. 6). By SDS-PAGE analysis, the main product was identified at about 150 kDa, and the byproduct was not identified (FIG. 7). It was found that the presence or absence of a linker between the CL domain and the hinge region had no significant effect on the formation of the byproduct.

Based on these results, it was found that the Fab-(Fc)₂ structure was stably formed when the VH 44 and VL 100 positions of Fab were substituted with respective cysteines.

TABLE 7

| Name | Polypeptide sequence | SEQ ID NO |
|---|---|---|
| Trastuzumab VH- | EVQLVESGGGLVQPGGSLRLSCAASGFNIKDTYIHWVRQAPGKGLEW | 1 |
| CH1 domain | VARIYPTNGYTRYADSVKGRFTISADTSKNTAYLQMNSLRAEDTAVY | |
| | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKV | |
| Trastuzumab VL- | DIQMTQSPSSLSASVGDRVTITCRASQDVNTAVAWYQQKPGKAPKLL | 2 |
| CL domain | IYSASFLYSGVPSRFSGSRSGTDFTLTISSLQPEDFATYYCQQHYTTPPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| IgG1 Fc(CH2- | APELLGGPSVFLFPPKPKDTLMISRTPEVTCVVVDVSHEDPEVKFNWY | 3 |
| CH3) | VDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQ | |
| | QGNVFSCSVMHEALHNHYTQKSLSLSPGK | |
| IgG1 Fc(Knob; | APELLGGPSVFLFPPKPKDTLMISRTPEVTCVVVDVSHEDPEVKFNWY | 4 |
| S354C, T366W) | VDGVEVHNAKTKPREEQYNSTYRVVSVLTVLHQDWLNGKEYKCKV | |
| | SNKALPAPIEKTISKAKGQPREPQVYTLPPCREEMTKNQVSLWCLVKG | |
| | FYPSDIAVEWESNGQPENNYKTTPPVLDSDGSFFLYSKLTVDKSRWQ | |
| | QGNVFSCSVMHEALHNHYTQKSLSLSPGK | |
| IgG1 Fc(Hole; | APELLGGPSVFLFPPKPKDTLMISRTPEVTCVVVDVSHEDPEVKFNWY | 5 |
| Y349C, T366S, | VDGVEVHNAKTKPREEQYNSTYRVVSVLTVLHQDWLNGKEYKCKV | |
| L368A, Y407V) | SNKALPAPIEKTISKAKGQPREPQVCTLPPSREEMTKNQVSLSCAVKG | |
| | FYPSDIAVEWESNGQPENNYKTTPPVLDSDGSFFLVSKLTVDKSRWQ | |
| | QGNVFSCSVMHEALHNHYTQKSLSLSPGK | |
| 15-mer Linker | GGGGSGGGGSGGGGS | 6 |
| Fc-Hole | DKTHTCPPCPAPELLGGPSVFLFPPKPKDTLMISRTPEVTCVVVDVSHE | 7 |
| | DPEVKFNWYVDGVEVHNAKTKPREEQYNSTYRVVSVLTVLHQDWL | |
| | NGKEYKCKVSNKALPAPIEKTISKAKGQPREPQVCTLPPSREEMTKNQ | |
| | VSLSCAVKGFYPSDIAVEWESNGQPENNYKTTPPVLDSDGSFFLVSKL | |
| | TVDKSRWQQGNVFSCSVMHEALHNHYTQKSLSLSPGK | |
| TraH-WT-Knob | EVQLVESGGGLVQPGGSLRLSCAASGFNIKDTYIHWVRQAPGKGLEW | 8 |
| | VARIYPTNGYTRYADSVKGRFTISADTSKNTAYLQMNSLRAEDTAVY | |
| | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| TraL-WT-Knob | DIQMTQSPSSLSASVGDRVTITCRASQDVNTAVAWYQQKPGKAPKLL | 9 |
| | IYSASFLYSGVPSRFSGSRSGTDFTLTISSLQPEDFATYYCQQHYTTPPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGECGGGGGGGGSGGGGSEPKSSDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPCREEMTKNQVSL | |
| | WCLVKGFYPSDIAVEWESNGQPENNYKTTPPVLDSDGSFFLYSKLTV | |
| | DKSRWQQGNVFSCSVMHEALHNHYTQKSLSLSPGK | |
| TraH-Q105C- | EVQLVESGGGLVQPGGSLRLSCAASGFNIKDTYIHWVRQAPGKGLEW | 10 |
| Knob | VARIYPTNGYTRYADSVKGRFTISADTSKNTAYLQMNSLRAEDTAVY | |
| | YCSRWGGDGFYAMDYWGCGTLVTVSSASTKGPSVFPLAPSSKSTSGG | |
| | TAALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCPAPE | |
| | LLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| TraL-A43C-Knob | DIQMTQSPSSLSASVGDRVTITCRASQDVNTAVAWYQQKPGKCPKLLI | 11 |
| | YSASFLYSGVPSRFSGSRSGTDFTLTISSLQPEDFATYYCQQHYTTPPTF | |
| | GQGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECGGGGSGGGGSGGGGSEPKSSDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKS | |
| | RWQQGNVFSCSVMHEALHNHYTQKSLSLSPGK | |
| TraH-F122C- | EVQLVESGGGLVQPGGSLRLSCAASGFNIKDTYIHWVRQAPGKGLEW | 12 |
| Knob | VARIYPTNGYTRYADSVKGRFTISADTSKNTAYLQMNSLRAEDTAVY | |
| | YCSRWGGDGFYAMDYWGQGTLVTVSSASTKGPSVCPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| TraL-S121C- | DIQMTQSPSSLSASVGDRVTITCRASQDVNTAVAWYQQKPGKAPKLL | 13 |
| Knob | IYSASFLYSGVPSRFSGSRSGTDFTLTISSLQPEDFATYYCQQHYTTPPT | |
| | FGQGTKVEIKRTVAAPSVFIFPPCDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGECGGGGSGGGGSGGGGSEPKSSDKTH | |
| | TCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALPAPIEKTISKAKGQPREPQVYTLPPCREEMTKNQVSL | |
| | WCLVKGFYPSDIAVEWESNGQPENNYKTTPPVLDSDGSFFLYSKLTV | |
| | DKSRWQQGNVFSCSVMHEALHNRFTQKSLSLSPGK | |
| TraH-G44C-Knob | EVQLVESGGGLVQPGGSLRLSCAASGFNIKDTYIHWVRQAPGKCLEW | 14 |
| | VARIYPTNGYTRYADSVKGRFTISADTSKNTAYLQMNSLRAEDTAVY | |
| | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| TraL-Q100C- | DIQMTQSPSSLSASVGDRVTITCRASQDVNTAVAWYQQKPGKAPKLL | 15 |
| Knob | IYSASFLYSGVPSRFSGSRSGTDFTLTISSLQPEDFATYYCQQHYTTPPT | |
| | FGCGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECGGGGSGGGGSGGGGSEPKSSDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKS | |
| | RWQQGNVFSCSVMHEALHNHYTQKSLSLSPGK | |
| TraL-Q100C- | DIQMTQSPSSLSASVGDRVTITCRASQDVNTAVAWYQQKPGKAPKLL | 16 |
| Knob1 | IYSASFLYSGVPSRFSGSRSGTDFTLTISSLQPEDFATYYCQQHYTTPPT | |
| | FGCGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECGGGGSGGGGSEPKSSDKTHTCPPCPA | |
| | PELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVS | |
| | NKALPAPIEKTISKAKGQPREPQVYTLPPCREEMTKNQVSLWCL VKGF | |
| | YPSDIAVEWESNGQPENNYKTTPPVLDSDGSFFLYSKLTVDKSRWQQ | |
| | GNVFSCSVMHEALHNHYTQKSLSLSPGK | |
| TraL-Q100C- | DIQMTQSPSSLSASVGDRVTITCRASQDVNTAVAWYQQKPGKAPKLL | 17 |
| Knob2 | IYSASFLYSGVPSRFSGSRSGTDFTLTISSLQPEDFATYYCQQHYTTPPT | |
| | FGCGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECGGGGSEPKSSDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPCREEMTKNQVSLWCL VKGFYPSDI | |
| | AVEWESNGQPENNYKTTPPVLDSDGSFFLYSKLTVDKSRWQQGNVFS | |
| | CSVMHEALHNHYTQKSLSLSPGK | |
| TraL-Q100C- | DIQMTQSPSSLSASVGDRVTITCRASQDVNTAVAWYQQKPGKAPKLL | 18 |
| Knob3 | IYSASFLYSGVPSRFSGSRSGTDFTLTISSLQPEDFATYYCQQHYTTPPT | |
| | FGCGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECEPKSSDKTHTCPPCPAPELLGGPSVFL | |
| | FPPKPKDTLMISRTPEVTCVVVDVSHEDPEVKFNWYVDGVEVHNAKT | |
| | KPREEQYNSTYRVVSVLTVLHQDWLNGKEYKCKVSNKALPAPIEKTI | |
| | SKAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMHE | |
| | ALHNHYTQKSLSLSPGK | |

TABLE 8

| Name | Nucleotide sequence | SEQ ID NO |
|---|---|---|
| Trastuzumab VH- | GAAGTGCAGCTGGTCGAAAGTGGCGGTGGACTTGTGCAACCTGGCGG | 452 |
| CH1 domain | TAGCCTCCGTCTCAGCTGCGCTGCAAGTGGGTTCAACATCAAGGACA | |
| | CTTATATTCATTGGGTCCGACAGGCACCTGGGAAAGGTTTGGAGTGG | |
| | GTCGCACGGATCTATCCCACTAATGGTTACACAAGATATGCCGATTC | |
| | AGTAAAAGGCCGGTTTACAATCAGCGCAGATACTTCAAAAAACACTG | |
| | CCTATCTTCAAATGAACTCACTTCGAGCAGAAGACACAGCCGTCTAT | |
| | TATTGTAGTCGTTGGGGAGGCGACGGCTTTTATGCTATGGACTACTGG | |
| | GGACAAGGAACTCTGGTCACAGTTTCATCAGCTAGCACCAAAGGACC | |
| | TAGTGTTTTTCCTCTTGCCCCTTCCTCAAAGTCTACCTCTGGGGGGAC | |
| | AGCCGCTCTGGGCTGCCTGGTCAAGGATTATTTCCCAGAGCCTGTCAC | |
| | TGTCAGTTGGAACTCTGGAGCCTTGACTTCTGGTGTTCATACATTTCC | |
| | TGCTGTCCTTCAAAGCAGCGGCTTGTACTCATTGTCTTCTGTTGTGAC | |
| | AGTACCCTCAAGCAGCCTCGGCACTCAGACATACATCTGCAATGTCA | |
| | ACCACAAACCCTCAAATACAAAGGTAGATAAAAAAGTC | |
| Trastuzumab VL- | GATATTCAGATGACTCAGAGTCCTAGTTCCCTCAGCGCCTCCGTAGGC | 453 |
| CL domain | GACAGAGTTACAATAACTTGCCGAGCAAGCCAAGACGTAAACACTGC | |
| | AGTCGCCTGGTACCAACAGAAACCAGGCAAAGCTCCAAAACTCTTGA | |
| | TTTACAGTGCTTCCTTCCTTTATAGTGGCGTTCCAAGCCGCTTCAGCG | |
| | GCAGCCGCTCTGGCACCGACTTCACTCTCACTATTTCTTCCTTGCAAC | |
| | CTGAAGACTTCGCCACTTATTATTGCCAGCAACACTACACAACACCC | |
| | CCAACATTCGGACAGGGCACAAAGGTAGAAATAAAACGTACGGTGG | |
| | CAGCTCCCAGCGTTTTTATCTTTCCCCCATCCGACGAGCAGCTCAAGA | |
| | GTGGCACTGCCTCTGTAGTTTGTTTGCTGAATAACTTCTATCCACGTG | |
| | AAGCAAAAGTACAGTGGAAGGTCGATAATGCCCTTCAGAGCGGTAA | |
| | CAGCCAAGAAAGTGTTACCGAGCAAGATTCCAAAGATTCCACTTACA | |
| | GTCTGTCCAGCACATTGACACTGAGTAAGGCTGATTACGAAAAACAC | |
| | AAGGTGTACGCATGCGAGGTGACACACCAAGGTCTTTCATCTCCTGT | |
| | AACTAAGAGCTTTAACCGGGGAGAATGT | |
| IgG1 Fc(CH2- | GCACCTGAACTCCTGGGGGGACCGTCAGTCTTCCTCTTCCCCCCAAAA | 454 |
| CH3) | CCCAAGGACACCCTCATGATCTCCCGGACCCCTGAGGTCACATGCGT | |
| | GGTGGTGGACGTGAGCCACGAAGACCCTGAGGTCAAGTTCAACTGGT | |
| | ACGTGGACGGCGTGGAGGTGCATAATGCCAAGACAAAGCCGCGGGA | |
| | GGAGCAGTACAACAGCACGTACCGTGTGGTCAGCGTCCTCACCGTCC | |
| | TGCACCAGGACTGGCTGAATGGCAAGGAGTACAAGTGCAAGGTCTCC | |
| | AACAAAGCCCTCCCAGCCCCCATCGAGAAAACCATCTCCAAAGCCAA | |
| | AGGGCAGCCCCGAGAACCACAGGTGTACACCCTGCCCCCAAGCCGG | |
| | GAGGAGATGACCAAGAACCAGGTCAGCCTGACGTGCCTGGTCAAAG | |
| | GCTTCTATCCCAGCGACATCGCCGTGGAGTGGGAGAGCAATGGGCAG | |
| | CCGGAGAACAACTACAAGACCACGCCTCCCGTGCTGGACTCCGACGG | |
| | CTCCTTCTTCCTCTACAGCAAGCTCACCGTGGACAAGAGCAGGTGGC | |
| | AGCAGGGGAACGTCTTCTCATGCTCCGTGATGCATGAGGCTCTGCAC | |
| | AACCACTACACGCAGAAGAGCCTCTCCCTGTCTCCGGGTAAA | |
| IgG1 Fc(Knob; | GCACCTGAACTCCTGGGGGGACCGTCAGTCTTCCTCTTCCCCCCAAAA | 455 |
| S354C, T366W) | CCCAAGGACACCCTCATGATCTCCCGGACCCCTGAGGTCACATGCGT | |
| | GGTGGTGGACGTGAGCCACGAAGACCCTGAGGTCAAGTTCAACTGGT | |
| | ACGTGGACGGCGTGGAGGTGCATAATGCCAAGACAAAGCCGCGGGA | |
| | GGAGCAGTACAACAGCACGTACCGTGTGGTCAGCGTCCTCACCGTCC | |
| | TGCACCAGGACTGGCTGAATGGCAAGGAGTACAAGTGCAAGGTCTCC | |
| | AACAAAGCCCTCCCAGCCCCCATCGAGAAAACCATCTCCAAAGCCAA | |
| | AGGGCAGCCCCGAGAACCACAGGTGTACACCCTGCCCCCATGCCGGG | |
| | AGGAGATGACCAAGAACCAGGTCAGCCTGTGGTGCCTGGTCAAAGG | |
| | CTTCTATCCCAGCGACATCGCCGTGGAGTGGGAGAGCAATGGGCAGC | |
| | CGGAGAACAACTACAAGACCACGCCTCCCGTGCTGGACTCCGACGGC | |
| | TCCTTCTTCCTCTACAGCAAGCTCACCGTGGACAAGAGCAGGTGGCA | |
| | GCAGGGGAACGTCTTCTCATGCTCCGTGATGCATGAGGCTCTGCACA | |
| | ACCACTACACGCAGAAGAGCCTCTCCCTGTCTCCGGGTAAA | |
| IgG1 Fc(Hole; | GCACCTGAACTCCTGGGGGGACCGTCAGTCTTCCTCTTCCCCCCAAAA | 456 |
| Y349C, T366S, | CCCAAGGACACCCTCATGATCTCCCGGACCCCTGAGGTCACATGCGT | |
| L368A, Y407V) | GGTGGTGGACGTGAGCCACGAAGACCCTGAGGTCAAGTTCAACTGGT | |
| | ACGTGGACGGCGTGGAGGTGCATAATGCCAAGACAAAGCCGCGGGA | |
| | GGAGCAGTACAACAGCACGTACCGTGTGGTCAGCGTCCTCACCGTCC | |
| | TGCACCAGGACTGGCTGAATGGCAAGGAGTACAAGTGCAAGGTCTCC | |
| | AACAAAGCCCTCCCAGCCCCCATCGAGAAAACCATCTCCAAAGCCAA | |
| | AGGGCAGCCCCGAGAACCACAGGTGTGCACCCTGCCCCCATCCCGGG | |
| | AGGAGATGACCAAGAACCAGGTCAGCCTGAGCTGCGCGGTCAAAGG | |
| | CTTCTATCCCAGCGACATCGCCGTGGAGTGGGAGAGCAATGGGCAGC | |
| | CGGAGAACAACTACAAGACCACGCCTCCCGTGCTGGACTCCGACGGC | |
| | TCCTTCTTCCTCGTGAGCAAGCTCACCGTGGACAAGAGCAGGTGGCA | |
| | GCAGGGGAACGTCTTCTCATGCTCCGTGATGCATGAGGCTCTGCACA | |
| | ACCACTACACGCAGAAGAGCCTCTCCCTGTCTCCGGGTAAA | |
| 15-mer Linker | GGTGGTGGGGGCAGCGGGGGCGGAGGTAGTGGAGGCGGCGGTAGT | 457 |
| Fc-Hole | GACAAAACTCACACATGCCCACCGTGCCCAGCACCTGAACTCCTGGG | 458 |
| | GGGACCGTCAGTCTTCCTCTTCCCCCCAAAACCCAAGGACACCCTCAT | |
| | GATCTCCCGGACCCCTGAGGTCACATGCGTGGTGGTGGACGTGAGCC | |
| | ACGAAGACCCTGAGGTCAAGTTCAACTGGTACGTGGACGGCGTGGAG | |
| | GTGCATAATGCCAAGACAAAGCCGCGGGAGGAGCAGTACAACAGCA | |
| | CGTACCGTGTGGTCAGCGTCCTCACCGTCCTGCACCAGGACTGGCTG | |
| | AATGGCAAGGAGTACAAGTGCAAGGTCTCCAACAAAGCCCTCCCAGC | |
| | CCCCATCGAGAAAACCATCTCCAAAGCCAAAGGGCAGCCCCGAGAA | |
| | CCACAGGTGTGCACCCTGCCCCCATCCCGGGAGGAGATGACCAAGAA | |
| | CCAGGTCAGCCTGAGCTGCGCGGTCAAAGGCTTCTATCCCAGCGACA | |
| | TCGCCGTGGAGTGGGAGAGCAATGGGCAGCCGGAGAACAACTACAA | |
| | GACCACGCCTCCCGTGCTGGACTCCGACGGCTCCTTCTTCCTCGTGAG | |
| | CAAGCTCACCGTGGACAAGAGCAGGTGGCAGCAGGGGAACGTCTTCT | |
| | CATGCTCCGTGATGCATGAGGCTCTGCACAACCACTACACGCAGAAG | |
| | AGCCTCTCCCTGTCTCCGGGTAAA | |
| TraH-WT-Knob | GAAGTTCAACTTGTGGAGTCTGGAGGCGGTTTGGTACAACCTGGAGG | 459 |
| | TTCACTTCGTTTGTCATGTGCTGCCTCTGGGTTTAATATCAAGGATAC | |
| | TTATATTCATTGGGTTCGCCAGGCACCCGGAAAAGGGCTGGAATGGG | |
| | TGGCCCGTATATATCCCACTAACGGTTACACTCGATATGCTGACTCTG | |
| | TAAAGGGTCGATTTACTATATCCGCTGATACTAGCAAAAACACTGCC | |
| | TACCTCCAGATGAACAGTCTGCGCGCCGAGGATACAGCAGTTTATTA | |
| | TTGTTCCCGATGGGGAGGTGACGGGTTCTATGCCATGGACTACTGGG | |
| | GTCAAGGGACACTGGTAACCGTTTCTTCTGCTAGCACCAAAGGACCT | |
| | AGTGTTTTTCCTCTTGCCCCTTCCTCAAAGTCTACCTCTGGGGGGACA | |
| | GCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCT | |
| | GCTGTCCTTCAAAGCAGCGGCTTGTACTCATTGTCTTCTGTTGTGACA | |
| | GTACCCTCAAGCAGCCTCGGCACTCAGACATACATCTGCAATGTCAA | |
| | CCACAAACCCTCAAATACAAAGGTAGATAAAAAAGTCGAACCAAAG | |
| | TCTTGTGACAAAACTCACACGTGCCCACCGTGCCCAGCACCTGAACT | |
| | CCTGGGGGGACCGTCAGTCTTCCTCTTCCCCCCAAAACCCAAGGACA | |
| | CCCTCATGATCTCCCGGACCCCTGAGGTCACATGCGTGGTGGTGGAC | |
| | GTGAGCCACGAAGACCCTGAGGTCAAGTTCAACTGGTACGTGGACGG | |
| | CGTGGAGGTGCATAATGCCAAGACAAAGCCGCGGGAGGAGCAGTAC | |
| | AACAGCACGTACCGTGTGGTCAGCGTCCTCACCGTCCTGCACCAGGA | |
| | CTGGCTGAATGGCAAGGAGTACAAGTGCAAGGTCTCCAACAAAGCCC | |
| | TCCCAGCCCCCATCGAGAAAACCATCTCCAAAGCCAAAGGGCAGCCC | |
| | CGAGAACCACAGGTGTACACCCTGCCCCCATGCCGGGAGGAGATGAC | |
| | CAAGAACCAGGTCAGCCTGTGGTGCCTGGTCAAAGGCTTCTATCCCA | |
| | GCGACATCGCCGTGGAGTGGGAGAGCAATGGGCAGCCGGAGAACAA | |
| | CTACAAGACCACGCCTCCCGTGCTGGACTCCGACGGCTCCTTCTTCCT | |
| | CTACAGCAAGCTCACCGTGGACAAGAGCAGGTGGCAGCAGGGGAAC | |
| | GTCTTCTCATGCTCCGTGATGCATGAGGCTCTGCACAACCACTACACG | |
| | CAGAAGAGCCTCTCCCTGTCTCCGGGTAAA | |
| TraL-WT-Knob | GACATTCAGATGACCCAATCCCCCTCTAGCCTTTCAGCCTCCGTAGGG | 460 |
| | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGACAGGGCACAAAAGTAGAGATTAAGCGTACGGTGGC | |
| | AGCTCCCAGCGTTTTTATCTTTCCCCCATCCGACGAGCAGCTCAAGAG | |
| | TGGCACTGCCTCTGTAGTTTGTTTGCTGAATAACTTCTATCCACGTGA | |
| | AGCAAAAGTACAGTGGAAGGTCGATAATGCCCTTCAGAGCGGTAAC | |
| | AGCCAAGAAAGTGTTACCGAGCAAGATTCCAAAGATTCCACTTACAG | |
| | TCTGTCCAGCACATTGACACTGAGTAAGGCTGATTACGAAAAACACA | |
| | AGGTGTACGCATGCGAGGTGACACACCAAGGTCTTTCATCTCCTGTA | |
| | ACTAAGAGCTTTAACCGGGGAGAATGTGGTGGTGGGGGCAGCGGGG | |
| | GCGGAGGTAGTGGAGGCGGCGGTAGTGAACCAAAGAGTAGTGACAA | |
| | AACTCACACGTGCCCACCGTGCCCAGCACCTGAACTCCTGGGGGGAC | |
| | CGTCAGTCTTCCTCTTCCCCCCAAAACCCAAGGACACCCTCATGATCT | |
| | CCCGGACCCCTGAGGTCACATGCGTGGTGGTGGACGTGAGCCACGAA | |
| | GACCCTGAGGTCAAGTTCAACTGGTACGTGGACGGCGTGGAGGTGCA | |
| | TAATGCCAAGACAAAGCCGCGGGAGGAGCAGTACAACAGCACGTAC | |
| | CGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCA | |
| | TCGAGAAAACCATCTCCAAAGCCAAAGGGCAGCCCCGAGAACCACA | |
| | GGTGTACACCCTGCCCCCATGCCGGGAGGAGATGACCAAGAACCAG | |
| | GTCAGCCTGTGGTGCCTGGTCAAAGGCTTCTATCCCAGCGACATCGC | |
| | CGTGGAGTGGGAGAGCAATGGGCAGCCGGAGAACAACTACAAGACC | |
| | ACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATG | |
| | CTCCGTGATGCATGAGGCTCTGCACAACCACTACACGCAGAAGAGCC | |
| | TCTCCCTGTCTCCGGGTAAA | |
| TraH-Q105C- | GAAGTTCAACTTGTGGAGTCTGGAGGCGGTTTGGTACAACCTGGAGG | 461 |
| Knob | TTCACTTCGTTTGTCATGTGCTGCCTCTGGGTTTAATATCAAGGATAC | |
| | TTATATTCATTGGGTTCGCCAGGCACCCGGAAAAGGGCTGGAATGGG | |
| | TGGCCCGTATATATCCCACTAACGGTTACACTCGATATGCTGACTCTG | |
| | TAAAGGGTCGATTTACTATATCCGCTGATACTAGCAAAAACACTGCC | |
| | TACCTCCAGATGAACAGTCTGCGCGCCGAGGATACAGCAGTTTATTA | |
| | TTGTTCCCGATGGGGAGGTGACGGGTTCTATGCCATGGACTACTGGG | |
| | GTTGTGGGACACTGGTAACCGTTTCTTCTGCTAGCACCAAAGGACCT | |
| | AGTGTTTTTCCTCTTGCCCCTTCCTCAAAGTCTACCTCTGGGGGGACA | |
| | GCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCT | |
| | GCTGTCCTTCAAAGCAGCGGCTTGTACTCATTGTCTTCTGTTGTGACA | |
| | GTACCCTCAAGCAGCCTCGGCACTCAGACATACATCTGCAATGTCAA | |
| | CCACAAACCCTCAAATACAAAGGTAGATAAAAAAGTCGAACCAAAG | |
| | TCTTGTGACAAAACTCACACGTGCCCACCGTGCCCAGCACCTGAACT | |
| | CCTGGGGGGACCGTCAGTCTTCCTCTTCCCCCCAAAACCCAAGGACA | |
| | CCCTCATGATCTCCCGGACCCCTGAGGTCACATGCGTGGTGGTGGAC | |
| | GTGAGCCACGAAGACCCTGAGGTCAAGTTCAACTGGTACGTGGACGG | |
| | CGTGGAGGTGCATAATGCCAAGACAAAGCCGCGGGAGGAGCAGTAC | |
| | AACAGCACGTACCGTGTGGTCAGCGTCCTCACCGTCCTGCACCAGGA | |
| | CTGGCTGAATGGCAAGGAGTACAAGTGCAAGGTCTCCAACAAAGCCC | |
| | TCCCAGCCCCCATCGAGAAAACCATCTCCAAAGCCAAAGGGCAGCCC | |
| | CGAGAACCACAGGTGTACACCCTGCCCCCATGCCGGGAGGAGATGAC | |
| | CAAGAACCAGGTCAGCCTGTGGTGCCTGGTCAAAGGCTTCTATCCCA | |
| | GCGACATCGCCGTGGAGTGGGAGAGCAATGGGCAGCCGGAGAACAA | |
| | CTACAAGACCACGCCTCCCGTGCTGGACTCCGACGGCTCCTTCTTCCT | |
| | CTACAGCAAGCTCACCGTGGACAAGAGCAGGTGGCAGCAGGGGAAC | |
| | GTCTTCTCATGCTCCGTGATGCATGAGGCTCTGCACAACCACTACACG | |
| | CAGAAGAGCCTCTCCCTGTCTCCGGGTAAA | |
| TraL-A43C-Knob | GACATTCAGATGACCCAATCCCCCTCTAGCCTTTCAGCCTCCGTAGGG | 462 |
| | GACAGAGTTACAATAACCTGTAGGGCTAGTCAGGACGTTAATACAGC | |
| | AGTCGCATGGTACCAGCAGAAGCCCGGTAAGTGTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGACAGGGCACAAAAGTAGAGATTAAGCGTACGGTGGC | |
| | AGCTCCCAGCGTTTTTATCTTTCCCCCATCCGACGAGCAGCTCAAGAG | |
| | TGGCACTGCCTCTGTAGTTTGTTTGCTGAATAACTTCTATCCACGTGA | |
| | AGCAAAAGTACAGTGGAAGGTCGATAATGCCCTTCAGAGCGGTAAC | |
| | AGCCAAGAAAGTGTTACCGAGCAAGATTCCAAAGATTCCACTTACAG | |
| | TCTGTCCAGCACATTGACACTGAGTAAGGCTGATTACGAAAAACACA | |
| | AGGTGTACGCATGCGAGGTGACACACCAAGGTCTTTCATCTCCTGTA | |
| | ACTAAGAGCTTTAACCGGGGAGAATGTGGTGGTGGGGGCAGCGGGG | |
| | GCGGAGGTAGTGGAGGCGGCGGTAGTGAACCAAAGAGTAGTGACAA | |
| | AACTCACACGTGCCCACCGTGCCCAGCACCTGAACTCCTGGGGGGAC | |
| | CGTCAGTCTTCCTCTTCCCCCCAAAACCCAAGGACACCCTCATGATCT | |
| | CCCGGACCCCTGAGGTCACATGCGTGGTGGTGGACGTGAGCCACGAA | |
| | GACCCTGAGGTCAAGTTCAACTGGTACGTGGACGGCGTGGAGGTGCA | |
| | TAATGCCAAGACAAAGCCGCGGGAGGAGCAGTACAACAGCACGTAC | |
| | CGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCA | |
| | TCGAGAAAACCATCTCCAAAGCCAAAGGGCAGCCCCGAGAACCACA | |
| | GGTGTACACCCTGCCCCCATGCCGGGAGGAGATGACCAAGAACCAG | |
| | GTCAGCCTGTGGTGCCTGGTCAAAGGCTTCTATCCCAGCGACATCGC | |
| | CGTGGAGTGGGAGAGCAATGGGCAGCCGGAGAACAACTACAAGACC | |
| | ACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATG | |
| | CTCCGTGATGCATGAGGCTCTGCACAACCACTACACGCAGAAGAGCC | |
| | TCTCCCTGTCTCCGGGTAAA | |
| TraH-F122C- | GAAGTTCAACTTGTGGAGTCTGGAGGCGGTTTGGTACAACCTGGAGG | 463 |
| Knob | TTCACTTCGTTTGTCATGTGCTGCCTCTGGGTTTAATATCAAGGATAC | |
| | TTATATTCATTGGGTTCGCCAGGCACCCGGAAAAGGGCTGGAATGGG | |
| | TGGCCCGTATATATCCCACTAACGGTTACACTCGATATGCTGACTCTG | |
| | TAAAGGGTCGATTTACTATATCCGCTGATACTAGCAAAAACACTGCC | |
| | TACCTCCAGATGAACAGTCTGCGCGCCGAGGATACAGCAGTTTATTA | |
| | TTGTTCCCGATGGGGAGGTGACGGGTTCTATGCCATGGACTACTGGG | |
| | GTCAAGGGACACTGGTAACCGTTTCTTCTGCTAGCACCAAAGGACCT | |
| | AGTGTTTGTCCTCTTGCCCCTTCCTCAAAGTCTACCTCTGGGGGGACA | |
| | GCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCT | |
| | GCTGTCCTTCAAAGCAGCGGCTTGTACTCATTGTCTTCTGTTGTGACA | |
| | GTACCCTCAAGCAGCCTCGGCACTCAGACATACATCTGCAATGTCAA | |
| | CCACAAACCCTCAAATACAAAGGTAGATAAAAAAGTCGAACCAAAG | |
| | TCTTGTGACAAAACTCACACGTGCCCACCGTGCCCAGCACCTGAACT | |
| | CCTGGGGGGACCGTCAGTCTTCCTCTTCCCCCCAAAACCCAAGGACA | |
| | CCCTCATGATCTCCCGGACCCCTGAGGTCACATGCGTGGTGGTGGAC | |
| | GTGAGCCACGAAGACCCTGAGGTCAAGTTCAACTGGTACGTGGACGG | |
| | CGTGGAGGTGCATAATGCCAAGACAAAGCCGCGGGAGGAGCAGTAC | |
| | AACAGCACGTACCGTGTGGTCAGCGTCCTCACCGTCCTGCACCAGGA | |
| | CTGGCTGAATGGCAAGGAGTACAAGTGCAAGGTCTCCAACAAAGCCC | |
| | TCCCAGCCCCCATCGAGAAAACCATCTCCAAAGCCAAAGGGCAGCCC | |
| | CGAGAACCACAGGTGTACACCCTGCCCCCATGCCGGGAGGAGATGAC | |
| | CAAGAACCAGGTCAGCCTGTGGTGCCTGGTCAAAGGCTTCTATCCCA | |
| | GCGACATCGCCGTGGAGTGGGAGAGCAATGGGCAGCCGGAGAACAA | |
| | CTACAAGACCACGCCTCCCGTGCTGGACTCCGACGGCTCCTTCTTCCT | |
| | CTACAGCAAGCTCACCGTGGACAAGAGCAGGTGGCAGCAGGGGAAC | |
| | GTCTTCTCATGCTCCGTGATGCATGAGGCTCTGCACAACCACTACACG | |
| | CAGAAGAGCCTCTCCCTGTCTCCGGGTAAA | |
| TraL-S121C- | GACATTCAGATGACCCAATCCCCCTCTAGCCTTTCAGCCTCCGTAGGG | 464 |
| Knob | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGACAGGGCACAAAAGTAGAGATTAAGCGTACGGTGGC | |
| | AGCTCCCAGCGTTTTTATCTTTCCCCCATGTGACGAGCAGCTCAAGAG | |
| | TGGCACTGCCTCTGTAGTTTGTTTGCTGAATAACTTCTATCCACGTGA | |
| | AGCAAAAGTACAGTGGAAGGTCGATAATGCCCTTCAGAGCGGTAAC | |
| | AGCCAAGAAAGTGTTACCGAGCAAGATTCCAAAGATTCCACTTACAG | |
| | TCTGTCCAGCACATTGACACTGAGTAAGGCTGATTACGAAAAACACA | |
| | AGGTGTACGCATGCGAGGTGACACACCAAGGTCTTTCATCTCCTGTA | |
| | ACTAAGAGCTTTAACCGGGGAGAATGTGGTGGTGGGGGCAGCGGGG | |
| | GCGGAGGTAGTGGAGGCGGCGGTAGTGAACCAAAGAGTAGTGACAA | |
| | AACTCACACGTGCCCACCGTGCCCAGCACCTGAACTCCTGGGGGGAC | |
| | CGTCAGTCTTCCTCTTCCCCCCAAAACCCAAGGACACCCTCATGATCT | |
| | CCCGGACCCCTGAGGTCACATGCGTGGTGGTGGACGTGAGCCACGAA | |
| | GACCCTGAGGTCAAGTTCAACTGGTACGTGGACGGCGTGGAGGTGCA | |
| | TAATGCCAAGACAAAGCCGCGGGAGGAGCAGTACAACAGCACGTAC | |
| | CGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCA | |
| | TCGAGAAAACCATCTCCAAAGCCAAAGGGCAGCCCCGAGAACCACA | |
| | GGTGTACACCCTGCCCCCATGCCGGGAGGAGATGACCAAGAACCAG | |
| | GTCAGCCTGTGGTGCCTGGTCAAAGGCTTCTATCCCAGCGACATCGC | |
| | CGTGGAGTGGGAGAGCAATGGGCAGCCGGAGAACAACTACAAGACC | |
| | ACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATG | |
| | CTCCGTGATGCATGAGGCTCTGCACAACAGATTTACGCAGAAGAGCC | |
| | TCTCCCTGTCTCCGGGTAAA | |
| TraH-G44C-Knob | GAAGTTCAACTTGTGGAGTCTGGAGGCGGTTTGGTACAACCTGGAGG | 465 |
| | TTCACTTCGTTTGTCATGTGCTGCCTCTGGGTTTAATATCAAGGATAC | |
| | TTATATTCATTGGGTTCGCCAGGCACCCGGAAAATGCCTGGAATGGG | |
| | TGGCCCGTATATATCCCACTAACGGTTACACTCGATATGCTGACTCTG | |
| | TAAAGGGTCGATTTACTATATCCGCTGATACTAGCAAAAACACTGCC | |
| | TACCTCCAGATGAACAGTCTGCGCGCCGAGGATACAGCAGTTTATTA | |
| | TTGTTCCCGATGGGGAGGTGACGGGTTCTATGCCATGGACTACTGGG | |
| | GTCAAGGGACACTGGTAACCGTTTCTTCTGCTAGCACCAAAGGACCT | |
| | AGTGTTTTTCCTCTTGCCCCTTCCTCAAAGTCTACCTCTGGGGGGACA | |
| | GCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCT | |
| | GCTGTCCTTCAAAGCAGCGGCTTGTACTCATTGTCTTCTGTTGTGACA | |
| | GTACCCTCAAGCAGCCTCGGCACTCAGACATACATCTGCAATGTCAA | |
| | CCACAAACCCTCAAATACAAAGGTAGATAAAAAAGTCGAACCAAAG | |
| | TCTTGTGACAAAACTCACACGTGCCCACCGTGCCCAGCACCTGAACT | |
| | CCTGGGGGGACCGTCAGTCTTCCTCTTCCCCCCAAAACCCAAGGACA | |
| | CCCTCATGATCTCCCGGACCCCTGAGGTCACATGCGTGGTGGTGGAC | |
| | GTGAGCCACGAAGACCCTGAGGTCAAGTTCAACTGGTACGTGGACGG | |
| | CGTGGAGGTGCATAATGCCAAGACAAAGCCGCGGGAGGAGCAGTAC | |
| | AACAGCACGTACCGTGTGGTCAGCGTCCTCACCGTCCTGCACCAGGA | |
| | CTGGCTGAATGGCAAGGAGTACAAGTGCAAGGTCTCCAACAAAGCCC | |
| | TCCCAGCCCCCATCGAGAAAACCATCTCCAAAGCCAAAGGGCAGCCC | |
| | CGAGAACCACAGGTGTACACCCTGCCCCCATGCCGGGAGGAGATGAC | |
| | CAAGAACCAGGTCAGCCTGTGGTGCCTGGTCAAAGGCTTCTATCCCA | |
| | GCGACATCGCCGTGGAGTGGGAGAGCAATGGGCAGCCGGAGAACAA | |
| | CTACAAGACCACGCCTCCCGTGCTGGACTCCGACGGCTCCTTCTTCCT | |
| | CTACAGCAAGCTCACCGTGGACAAGAGCAGGTGGCAGCAGGGGAAC | |
| | GTCTTCTCATGCTCCGTGATGCATGAGGCTCTGCACAACCACTACACG | |
| | CAGAAGAGCCTCTCCCTGTCTCCGGGTAAA | |
| TraL-Q100C- | GACATTCAGATGACCCAATCCCCCTCTAGCCTTTCAGCCTCCGTAGGG | 466 |
| Knob | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGATGTGGCACAAAAGTAGAGATTAAGCGTACGGTGGCA | |
| | GCTCCCAGCGTTTTTATCTTTCCCCCATCCGACGAGCAGCTCAAGAGT | |
| | GGCACTGCCTCTGTAGTTTGTTTGCTGAATAACTTCTATCCACGTGAA | |
| | GCAAAAGTACAGTGGAAGGTCGATAATGCCCTTCAGAGCGGTAACA | |
| | GCCAAGAAAGTGTTACCGAGCAAGATTCCAAAGATTCCACTTACAGT | |
| | CTGTCCAGCACATTGACACTGAGTAAGGCTGATTACGAAAAACACAA | |
| | GGTGTACGCATGCGAGGTGACACACCAAGGTCTTTCATCTCCTGTAA | |
| | CTAAGAGCTTTAACCGGGGAGAATGTGGTGGTGGGGGCAGCGGGGG | |
| | CGGAGGTAGTGGAGGCGGCGGTAGTGAACCAAAGAGTAGTGACAAA | |
| | ACTCACACGTGCCCACCGTGCCCAGCACCTGAACTCCTGGGGGGACC | |
| | GTCAGTCTTCCTCTTCCCCCCAAAACCCAAGGACACCCTCATGATCTC | |
| | CCGGACCCCTGAGGTCACATGCGTGGTGGTGGACGTGAGCCACGAAG | |
| | ACCCTGAGGTCAAGTTCAACTGGTACGTGGACGGCGTGGAGGTGCAT | |
| | AATGCCAAGACAAAGCCGCGGGAGGAGCAGTACAACAGCACGTACC | |
| | GTGTGGTCAGCGTCCTCACCGTCCTGCACCAGGACTGGCTGAATGGC | |
| | AAGGAGTACAAGTGCAAGGTCTCCAACAAAGCCCTCCCAGCCCCCAT | |
| | CGAGAAAACCATCTCCAAAGCCAAAGGGCAGCCCCGAGAACCACAG | |
| | GTGTACACCCTGCCCCCATGCCGGGAGGAGATGACCAAGAACCAGGT | |
| | CAGCCTGTGGTGCCTGGTCAAAGGCTTCTATCCCAGCGACATCGCCG | |
| | TGGAGTGGGAGAGCAATGGGCAGCCGGAGAACAACTACAAGACCAC | |
| | GCCTCCCGTGCTGGACTCCGACGGCTCCTTCTTCCTCTACAGCAAGCT | |
| | CACCGTGGACAAGAGCAGGTGGCAGCAGGGGAACGTCTTCTCATGCT | |
| | CCGTGATGCATGAGGCTCTGCACAACCACTACACGCAGAAGAGCCTC | |
| | TCCCTGTCTCCGGGTAAA | |
| TraL-Q100C- | GACATTCAGATGACCCAATCCCCCTCTAGCCTTTCAGCCTCCGTAGGG | 467 |
| Knob1 | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGATGTGGCACAAAAGTAGAGATTAAGCGTACGGTGGCA | |
| | GCTCCCAGCGTTTTTATCTTTCCCCCATCCGACGAGCAGCTCAAGAGT | |
| | GGCACTGCCTCTGTAGTTTGTTTGCTGAATAACTTCTATCCACGTGAA | |
| | GCAAAAGTACAGTGGAAGGTCGATAATGCCCTTCAGAGCGGTAACA | |
| | GCCAAGAAAGTGTTACCGAGCAAGATTCCAAAGATTCCACTTACAGT | |
| | CTGTCCAGCACATTGACACTGAGTAAGGCTGATTACGAAAAACACAA | |
| | GGTGTACGCATGCGAGGTGACACACCAAGGTCTTTCATCTCCTGTAA | |
| | CTAAGAGCTTTAACCGGGGAGAATGTGGTGGTGGGGGCAGCGGGGG | |
| | CGGAGGTAGTGAACCAAAGAGTAGTGACAAAACTCACACGTGCCCA | |
| | CCGTGCCCAGCACCTGAACTCCTGGGGGGACCGTCAGTCTTCCTCTTC | |
| | CCCCCAAAACCCAAGGACACCCTCATGATCTCCCGGACCCCTGAGGT | |
| | CACATGCGTGGTGGTGGACGTGAGCCACGAAGACCCTGAGGTCAAGT | |
| | TCAACTGGTACGTGGACGGCGTGGAGGTGCATAATGCCAAGACAAA | |
| | GCCGCGGGAGGAGCAGTACAACAGCACGTACCGTGTGGTCAGCGTCC | |
| | TCACCGTCCTGCACCAGGACTGGCTGAATGGCAAGGAGTACAAGTGC | |
| | AAGGTCTCCAACAAAGCCCTCCCAGCCCCCATCGAGAAAACCATCTC | |
| | CAAAGCCAAAGGGCAGCCCCGAGAACCACAGGTGTACACCCTGCCC | |
| | CCATGCCGGGAGGAGATGACCAAGAACCAGGTCAGCCTGTGGTGCCT | |
| | GGTCAAAGGCTTCTATCCCAGCGACATCGCCGTGGAGTGGGAGAGCA | |
| | ATGGGCAGCCGGAGAACAACTACAAGACCACGCCTCCCGTGCTGGAC | |
| | TCCGACGGCTCCTTCTTCCTCTACAGCAAGCTCACCGTGGACAAGAG | |
| | CAGGTGGCAGCAGGGGAACGTCTTCTCATGCTCCGTGATGCATGAGG | |
| | CTCTGCACAACCACTACACGCAGAAGAGCCTCTCCCTGTCTCCGGGT | |
| | AAA | |
| TraL-Q100C- | GACATTCAGATGACCCAATCCCCCTCTAGCCTTTCAGCCTCCGTAGGG | 468 |
| Knob2 | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGATGTGGCACAAAAGTAGAGATTAAGCGTACGGTGGCA | |
| | GCTCCCAGCGTTTTTATCTTTCCCCCATCCGACGAGCAGCTCAAGAGT | |
| | GGCACTGCCTCTGTAGTTTGTTTGCTGAATAACTTCTATCCACGTGAA | |
| | GCAAAAGTACAGTGGAAGGTCGATAATGCCCTTCAGAGCGGTAACA | |
| | GCCAAGAAAGTGTTACCGAGCAAGATTCCAAAGATTCCACTTACAGT | |
| | CTGTCCAGCACATTGACACTGAGTAAGGCTGATTACGAAAAACACAA | |
| | GGTGTACGCATGCGAGGTGACACACCAAGGTCTTTCATCTCCTGTAA | |
| | CTAAGAGCTTTAACCGGGGAGAATGTGGTGGTGGGGGCAGCGAACC | |
| | AAAGAGTAGTGACAAAACTCACACGTGCCCACCGTGCCCAGCACCTG | |
| | AACTCCTGGGGGGACCGTCAGTCTTCCTCTTCCCCCCAAAACCCAAG | |
| | GACACCCTCATGATCTCCCGGACCCCTGAGGTCACATGCGTGGTGGT | |
| | GGACGTGAGCCACGAAGACCCTGAGGTCAAGTTCAACTGGTACGTGG | |
| | ACGGCGTGGAGGTGCATAATGCCAAGACAAAGCCGCGGGAGGAGCA | |
| | GTACAACAGCACGTACCGTGTGGTCAGCGTCCTCACCGTCCTGCACC | |
| | AGGACTGGCTGAATGGCAAGGAGTACAAGTGCAAGGTCTCCAACAA | |
| | AGCCCTCCCAGCCCCCATCGAGAAAACCATCTCCAAAGCCAAAGGGC | |
| | AGCCCCGAGAACCACAGGTGTACACCCTGCCCCCATGCCGGGAGGAG | |
| | ATGACCAAGAACCAGGTCAGCCTGTGGTGCCTGGTCAAAGGCTTCTA | |
| | TCCCAGCGACATCGCCGTGGAGTGGGAGAGCAATGGGCAGCCGGAG | |
| | AACAACTACAAGACCACGCCTCCCGTGCTGGACTCCGACGGCTCCTT | |
| | CTTCCTCTACAGCAAGCTCACCGTGGACAAGAGCAGGTGGCAGCAGG | |
| | GGAACGTCTTCTCATGCTCCGTGATGCATGAGGCTCTGCACAACCACT | |
| | ACACGCAGAAGAGCCTCTCCCTGTCTCCGGGTAAA | |
| TraL-Q100C- | GACATTCAGATGACCCAATCCCCCTCTAGCCTTTCAGCCTCCGTAGGG | 469 |
| Knob3 | GACAGAGTTACAATAACCTGTAGGGCTAGTCAGGACGTTAATACAGC | |
| | AGTCGCATGGTACCAGCAGAAGCCCGGTAAGGCTCCTAAGCTTTTGA | |
| | TCTACAGTGCTTCTTTCCTGTATTCTGGTGTACCTAGCCGATTCTCAG | |
| | GCTCTCGGAGTGGCACAGACTTCACTCTGACAATTTCAAGTCTCCAGC | |
| | CAGAAGATTTTGCAACCTACTACTGCCAACAACACTACACTACTCCTC | |
| | CAACCTTCGGATGTGGCACAAAAGTAGAGATTAAGCGTACGGTGGCA | |
| | GCTCCCAGCGTTTTTATCTTTCCCCCATCCGACGAGCAGCTCAAGAGT | |
| | GGCACTGCCTCTGTAGTTTGTTTGCTGAATAACTTCTATCCACGTGAA | |
| | GCAAAAGTACAGTGGAAGGTCGATAATGCCCTTCAGAGCGGTAACA | |
| | GCCAAGAAAGTGTTACCGAGCAAGATTCCAAAGATTCCACTTACAGT | |
| | CTGTCCAGCACATTGACACTGAGTAAGGCTGATTACGAAAAACACAA | |
| | GGTGTACGCATGCGAGGTGACACACCAAGGTCTTTCATCTCCTGTAA | |
| | CTAAGAGCTTTAACCGGGGAGAATGTGAACCAAAGAGTAGTGACAA | |
| | AACTCACACGTGCCCACCGTGCCCAGCACCTGAACTCCTGGGGGGAC | |
| | CGTCAGTCTTCCTCTTCCCCCCAAAACCCAAGGACACCCTCATGATCT | |
| | CCCGGACCCCTGAGGTCACATGCGTGGTGGTGGACGTGAGCCACGAA | |
| | GACCCTGAGGTCAAGTTCAACTGGTACGTGGACGGCGTGGAGGTGCA | |
| | TAATGCCAAGACAAAGCCGCGGGAGGAGCAGTACAACAGCACGTAC | |
| | CGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCA | |
| | TCGAGAAAACCATCTCCAAAGCCAAAGGGCAGCCCCGAGAACCACA | |
| | GGTGTACACCCTGCCCCCATGCCGGGAGGAGATGACCAAGAACCAG | |
| | GTCAGCCTGTGGTGCCTGGTCAAAGGCTTCTATCCCAGCGACATCGC | |
| | CGTGGAGTGGGAGAGCAATGGGCAGCCGGAGAACAACTACAAGACC | |
| | ACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATG | |
| | CTCCGTGATGCATGAGGCTCTGCACAACCACTACACGCAGAAGAGCC | |
| | TCTCCCTGTCTCCGGGTAAA | |

Table 9 below shows the polypeptide sequences of the heavy chain variable region (VH) and light chain variable region (VL) of H01. Table 10 below shows the nucleotide sequences of the heavy chain variable region (VH) and light chain variable region (VL) of H01.

TABLE 9

| Name | Sequence | SEQ ID NO |
|---|---|---|
| H01 | EVQLVESGGGLVQPGGSLRLSCAASGFNIKDTYI | 19 |
| VH | HWVRQAPGKCLEWVARIYPTNGYTRYADSVKGRF | |
| | TISADTSKNTAYLQMNSLRAEDTAVYYCSRWGGD | |
| | GFYAMDYWGQGTLVTVSS | |
| H01 | DIQMTQSPSSLSASVGDRVTITCRASQDVNTAVA | 20 |
| VL | WYQQKPGKAPKLLIYSASFLYSGVPSRFSGSRSG | |
| | TDFTLTISSLQPEDFATYYCQQHYTTPPTFGCGT | |
| | KVEIK | |

TABLE 10

| Name | Sequence | SEQ ID NO |
|---|---|---|
| H01VH | GAAGTTCAACTTGTGGAGTCTGGAGGCGGTTTGGTACAACCTGGAG | 470 |
| | GTTCACTTCGTTTGTCATGTGCTGCCTCTGGGTTTAATATCAAGGAT | |
| | ACTTATATTCATTGGGTTCGCCAGGCACCCGGAAAATGCCTGGAAT | |
| | GGGTGGCCCGTATATATCCCACTAACGGTTACACTCGATATGCTGA | |
| | CTCTGTAAAGGGTCGATTTACTATATCCGCTGATACTAGCAAAAAC | |
| | ACTGCCTACCTCCAGATGAACAGTCTGCGCGCCGAGGATACAGCAG | |
| | TTTATTATTGTTCCCGATGGGGAGGTGACGGGTTCTATGCCATGGAC | |
| | TACTGGGGTCAAGGGACACTGGTAACCGTTTCTTCT | |
| H01VL | GACATTCAGATGACCCAATCCCCCTCTAGCCTTTCAGCCTCCGTAGG | 471 |
| | GGACAGAGTTACAATAACCTGTAGGGCTAGTCAGGACGTTAATACA | |
| | GCAGTCGCATGGTACCAGCAGAAGCCCGGTAAGGCTCCTAAGCTTT | |
| | TGATCTACAGTGCTTCTTTCCTGTATTCTGGTGTACCTAGCCGATTC | |
| | TCAGGCTCTCGGAGTGGCACAGACTTCACTCTGACAATTTCAAGTCT | |
| | CCAGCCAGAAGATTTTGCAACCTACTACTGCCAACAACACTACACT | |
| | ACTCCTCCAACCTTCGGATGTGGCACAAAAGTAGAGATTAAG | |

Table 11 below shows the H01 heavy chain and light chain CDR sequences.

TABLE 11

| Name | CDR region | Sequence | SEQ ID NO |
|---|---|---|---|
| H01 VH | CDR-H1 | DTYIH | 21 |
| | CDR-H2 | RIYPTNGYTRYADSVKG | 22 |
| | CDR-H3 | WGGDGFYAMDY | 23 |
| H01 VL | CDR-L1 | RASQDVNTAVA | 24 |
| | CDR-L2 | SASFLYS | 25 |
| | CDR-L3 | QQHYTTPPT | 26 |

### Example 2. Preparation of a Novel Antibody with Two Fc Domains Using Pertuzumab as a Template

M3 is characterized by a (trastuzumab Fab)-(Fc)₂ structure with mutations of VH G44C and VL Q100C, hereinafter referred to as H01. Similarly, based on the VH and VL regions of pertuzumab (SEQ ID NOs: 27 and 28), the (pertuzumab Fab)-(Fc)₂ structure with the mutations VH G44C and VL Q100C is hereinafter referred to as P01. For P01, expression vectors containing the sequences corresponding to Fc-Hole (SEQ ID NO: 7), PerH-G44C-Knob (SEQ ID NO: 29), and PerL-Q100C-Knob (SEQ ID NO: 30) were co-transfected into EXPICHO-S™ (Gibco, A29127), and purification and analysis were performed in the same manner as described in Example 1.

TABLE 12

| | | SEQ |
|---|---|---|
| Name | Sequence | ID NO |
| Pertuzumab | EVQLVESGGGLVQPGGSLRLSCAASGFTFTDYTMDWVRQAPGKGLE | 27 |
| VH domain | WVADVNPNSGGSIYNQRFKGRFTLSVDRSKNTLYLQMNSLRAEDTAV | |
| | YYCARNLGPSFYFDYWGQGTLVTVSS | |
| Pertuzumab | DIQMTQSPSSLSASVGDRVTITCKASQDVSIGVAWYQQKPGKAPKLLIY | 28 |
| VL domain | SASYRYTGVPSRFSGSGSGTDFTLTISSLQPEDFATYYCQQYYIYPYTFG | |
| | QGTKVEIK | |
| PerH-G44C- | EVQLVESGGGLVQPGGSLRLSCAASGFTFTDYTMDWVRQAPGKCLEW | 29 |
| Knob | VADVNPNSGGSIYNQRFKGRFTLSVDRSKNTLYLQMNSLRAEDTAVY | |
| | YCARNLGPSFYFDYWGQGTLVTVSSASTKGPSVFPLAPSSKSTSGGTA | |
| | ALGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPI | |
| | EKTISKAKGQPREPQVYTLPPCREEMTKNQVSLWCLVKGFYPSDIAVE | |
| | WESNGQPENNYKTTPPVLDSDGSFFLYSKLTVDKSRWQQGNVFSCSV | |
| | MHEALHNHYTQKSLSLSPGK | |
| PerL-Q100C- | DIQMTQSPSSLSASVGDRVTITCKASQDVSIGVAWYQQKPGKAPKLLIY | 30 |
| Knob | SASYRYTGVPSRFSGSGSGTDFTLTISSLQPEDFATYYCQQYYIYPYTFG | |
| | CGTKVEIKRTVAAPSVFIFPPSDEQLKSGTASVVCLLNNFYPREAKVQW | |
| | KVDNALQSGNSQESVTEQDSKDSTYSLSSTLTLSKADYEKHKVYACEV | |
| | THQGLSSPVTKSFNRGECGGGGSGGGGSGGGGSEPKSSDKTHTCPPCP | |
| | APELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVS | |
| | NKALPAPIEKTISKAKGQPREPQVYTLPPCREEMTKNQVSLWCLVKGF | |
| | YPSDIAVEWESNGQPENNYKTTPPVLDSDGSFFLYSKLTVDKSRWQQG | |
| | NVFSCSVMHEALHNHYTQKSLSLSPGK | |

TABLE 13

| Name | Sequence | SEQ ID NO |
|---|---|---|
| Pertuzumab VH | GAAGTGCAACTGGTGGAGTCTGGTGGTGGATTGGTGCAGCCAGGCG | 414 |
| domain | GTTCTCTGCGACTTAGTTGTGCAGCCTCCGGCTTTACCTTCACTGAT | |
| | TATACAATGGACTGGGTTCGGCAGGCACCCGGTAAGGGGCTTGAGT | |
| | GGGTCGCCGACGTCAATCCTAATTCAGGGGGAAGTATTTATAACCA | |
| | AAGGTTCAAGGGTCGATTTACATTGTCCGTAGATCGTAGTAAAAAT | |
| | ACCCTCTACCTTCAAATGAACTCCCTGAGGGCAGAGGATACCGCAG | |
| | TCTACTACTGCGCTCGTAACCTGGGGCCTAGTTTTTATTTCGATTAT | |
| | TGGGGCCAAGGCACATTGGTAACTGTGTCTTCA | |
| Pertuzumab VL | GATATACAAATGACACAATCTCCTAGTTCATTGAGTGCCTCAGTCG | 415 |
| domain | GCGACCGAGTCACTATAACTTGTAAAGCAAGCCAAGATGTTAGCAT | |
| | TGGCGTAGCTTGGTATCAGCAGAAACCTGGAAAAGCACCAAAACTG | |
| | CTTATCTACTCCGCTAGTTACCGTTACACCGGAGTTCCCTCAAGGTT | |
| | TTCTGGCAGCGGAAGTGGGACTGACTTCACTCTGACTATTTCTTCAC | |
| | TTCAGCCAGAAGACTTCGCTACTTATTACTGTCAGCAGTACTATATC | |
| | TATCCCTATACATTTGGACAAGGAACCAAAGTTGAGATTAAA | |
| PerH-G44C-Knob | GAAGTACAGTTGGTGGAGTCTGGTGGAGGTCTTGTACAACCTGGCG | 472 |
| | GGAGTTTGCGGCTTTCCTGCGCTGCAAGCGGGTTTACCTTCACCGAT | |
| | TATACCATGGATTGGGTACGCCAAGCCCCTGGTAAGTGCCTTGAGT | |
| | GGGTTGCCGATGTAAACCCTAATTCCGGAGGAAGTATCTATAATCA | |
| | ACGCTTCAAGGGCCGATTCACTCTGAGTGTGGATCGAAGCAAGAAC | |
| | ACCTTGTACTTGCAGATGAATTCCTTGCGGGCTGAAGACACAGCCG | |
| | TCTATTACTGCGCCCGAAATTTGGGGCCTTCATTCTATTTTGACTAT | |
| | TGGGGTCAGGGAACTCTGGTAACTGTTTCAAGTGCTAGCACCAAAG | |
| | GACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAGC | |
| | CTGTCACTGTCAGTTGGAACTCTGGAGCCTTGACTTCTGGTGTTCAT | |
| | ACATTTCCTGCTGTCCTTCAAAGCAGCGGCTTGTACTCATTGTCTTC | |
| | TGTTGTGACAGTACCCTCAAGCAGCCTCGGCACTCAGACATACATC | |
| | TGCAATGTCAACCACAAACCCTCAAATACAAAGGTAGATAAAAAA | |
| | GTCGAACCAAAGTCTTGTGACAAAACTCACACGTGCCCACCGTGCC | |
| | CAGCACCTGAACTCCTGGGGGGACCGTCAGTCTTCCTCTTCCCCCCA | |
| | AAACCCAAGGACACCCTCATGATCTCCCGGACCCCTGAGGTCACAT | |
| | GCGTGGTGGTGGACGTGAGCCACGAAGACCCTGAGGTCAAGTTCAA | |
| | CTGGTACGTGGACGGCGTGGAGGTGCATAATGCCAAGACAAAGCC | |
| | GCGGGAGGAGCAGTACAACAGCACGTACCGTGTGGTCAGCGTCCTC | |
| | ACCGTCCTGCACCAGGACTGGCTGAATGGCAAGGAGTACAAGTGCA | |
| | AGGTCTCCAACAAAGCCCTCCCAGCCCCCATCGAGAAAACCATCTC | |
| | CAAAGCCAAAGGGCAGCCCCGAGAACCACAGGTGTACACCCTGCC | |
| | CCCATGCCGGGAGGAGATGACCAAGAACCAGGTCAGCCTGTGGTG | |
| | CCTGGTCAAAGGCTTCTATCCCAGCGACATCGCCGTGGAGTGGGAG | |
| | AGCAATGGGCAGCCGGAGAACAACTACAAGACCACGCCTCCCGTG | |
| | CTGGACTCCGACGGCTCCTTCTTCCTCTACAGCAAGCTCACCGTGGA | |
| | CAAGAGCAGGTGGCAGCAGGGGAACGTCTTCTCATGCTCCGTGATG | |
| | CATGAGGCTCTGCACAACCACTACACGCAGAAGAGCCTCTCCCTGT | |
| | CTCCGGGTAAA | |
| PerL-Q100C- | GATATTCAAATGACCCAAAGTCCAAGTTCCCTTTCAGCATCTGTCGG | 473 |
| Knob | TGATCGAGTCACTATAACTTGTAAGGCCAGCCAAGACGTTAGCATA | |
| | GGAGTAGCATGGTACCAACAAAAGCCTGGGAAGGCTCCCAAACTTC | |
| | TCATTTATTCTGCTTCCTACCGATATACTGGTGTCCCAAGTAGATTT | |
| | TCTGGCAGCGGATCTGGAACTGATTTTACATTGACTATCAGCTCCCT | |
| | CCAGCCTGAGGACTTCGCTACTTATTACTGCCAACAGTACTACATTT | |
| | ATCCCTATACATTCGGTTGTGGGACCAAAGTAGAGATCAAACGTAC | |
| | GGTGGCAGCTCCCAGCGTTTTTATCTTTCCCCCATCCGACGAGCAGC | |
| | TCAAGAGTGGCACTGCCTCTGTAGTTTGTTTGCTGAATAACTTCTAT | |
| | CCACGTGAAGCAAAAGTACAGTGGAAGGTCGATAATGCCCTTCAGA | |
| | GCGGTAACAGCCAAGAAAGTGTTACCGAGCAAGATTCCAAAGATTC | |
| | CACTTACAGTCTGTCCAGCACATTGACACTGAGTAAGGCTGATTAC | |
| | GAAAAACACAAGGTGTACGCATGCGAGGTGACACACCAAGGTCTTT | |
| | CATCTCCTGTAACTAAGAGCTTTAACCGGGGAGAATGTGGTGGTGG | |
| | GGGCAGCGGGGGCGGAGGTAGTGGAGGCGGCGGTAGTGAACCAAA | |
| | GAGTAGTGACAAAACTCACACGTGCCCACCGTGCCCAGCACCTGAA | |
| | CTCCTGGGGGGACCGTCAGTCTTCCTCTTCCCCCCAAAACCCAAGG | |
| | ACACCCTCATGATCTCCCGGACCCCTGAGGTCACATGCGTGGTGGT | |
| | GGACGTGAGCCACGAAGACCCTGAGGTCAAGTTCAACTGGTACGTG | |
| | GACGGCGTGGAGGTGCATAATGCCAAGACAAAGCCGCGGGAGGAG | |
| | CAGTACAACAGCACGTACCGTGTGGTCAGCGTCCTCACCGTCCTGC | |
| | ACCAGGACTGGCTGAATGGCAAGGAGTACAAGTGCAAGGTCTCCA | |
| | ACAAAGCCCTCCCAGCCCCCATCGAGAAAACCATCTCCAAAGCCAA | |
| | AGGGCAGCCCCGAGAACCACAGGTGTACACCCTGCCCCCATGCCGG | |
| | GAGGAGATGACCAAGAACCAGGTCAGCCTGTGGTGCCTGGTCAAA | |
| | GGCTTCTATCCCAGCGACATCGCCGTGGAGTGGGAGAGCAATGGGC | |
| | AGCCGGAGAACAACTACAAGACCACGCCTCCCGTGCTGGACTCCGA | |
| | CGGCTCCTTCTTCCTCTACAGCAAGCTCACCGTGGACAAGAGCAGG | |
| | TGGCAGCAGGGGAACGTCTTCTCATGCTCCGTGATGCATGAGGCTC | |
| | TGCACAACCACTACACGCAGAAGAGCCTCTCCCTGTCTCCGGGTAA | |
| | A | |

Table 14 below shows the polypeptide sequences of the heavy chain variable region (VH) and light chain variable region (VL) of P01. Table 15 below shows the nucleotide sequences of the heavy chain variable region (VH) and light chain variable region (VL) of P01.

TABLE 14

| Name | Sequence | SEQ ID NO |
|---|---|---|
| P01 VH | EVQLVESGGGLVQPGGSLRLSCAASGFTFTDYTMDWVRQAPGKCLE | 31 |
| | WVADVNPNSGGSIYNQRFKGRFTLSVDRSKNTLYLQMNSLRAEDTA | |
| | VYYCARNLGPSFYFDYWGQGTLVTVSS | |
| P01 VL | DIQMTQSPSSLSASVGDRVTITCKASQDVSIGVAWYQQKPGKAPKLLI | 32 |
| | YSASYRYTGVPSRFSGSGSGTDFTLTISSLQPEDFATYYCQQYYIYPYT | |
| | FGCGTKVEIK | |

TABLE 15

| Name | Sequence | SEQ ID NO |
|---|---|---|
| P01 VH | GAAGTACAGTTGGTGGAGTCTGGTGGAGGTCTTGTACAACCTGGC | 474 |
| | GGGAGTTTGCGGCTTTCCTGCGCTGCAAGCGGGTTTACCTTCACCG | |
| | ATTATACCATGGATTGGGTACGCCAAGCCCCTGGTAAGTGCCTTGA | |
| | GTGGGTTGCCGATGTAAACCCTAATTCCGGAGGAAGTATCTATAAT | |
| | CAACGCTTCAAGGGCCGATTCACTCTGAGTGTGGATCGAAGCAAG | |
| | AACACCTTGTACTTGCAGATGAATTCCTTGCGGGCTGAAGACACAG | |
| | CCGTCTATTACTGCGCCCGAAATTTGGGGCCTTCATTCTATTTTGA | |
| | CTATTGGGGTCAGGGAACTCTGGTAACTGTTTCAAGT | |
| P01 VL | GATATTCAAATGACCCAAAGTCCAAGTTCCCTTTCAGCATCTGTCG | 475 |
| | GTGATCGAGTCACTATAACTTGTAAGGCCAGCCAAGACGTTAGCA | |
| | TAGGAGTAGCATGGTACCAACAAAAGCCTGGGAAGGCTCCCAAACT | |
| | TCTCATTTATTCTGCTTCCTACCGATATACTGGTGTCCCAAGTAGA | |
| | TTTTCTGGCAGCGGATCTGGAACTGATTTTACATTGACTATCAGCT | |
| | CCCTCCAGCCTGAGGACTTCGCTACTTATTACTGCCAACAGTACTA | |
| | CATTTATCCCTATACATTCGGTTGTGGGACCAAAGTAGAGATCAAA | |

Table 16 below shows CDR sequences in heavy and light chains of the P01.

TABLE 16

| Name | | Sequence | SEQ ID NO |
|---|---|---|---|
| P01 VH | CDR-H1 | DYTMD | 33 |
| | CDR-H2 | DVNPNSGGSIYNQRFKG | 34 |
| | CDR-H3 | NLGPSFYFDY | 35 |
| P01 VL | CDR-L1 | KASQDVSIGVA | 36 |
| | CDR-L2 | SASYRYT | 37 |
| | CDR-L3 | QQYYIYPYT | 38 |

### Example 3. Analysis of H01 and P01 Byproducts Through Papain Digestion

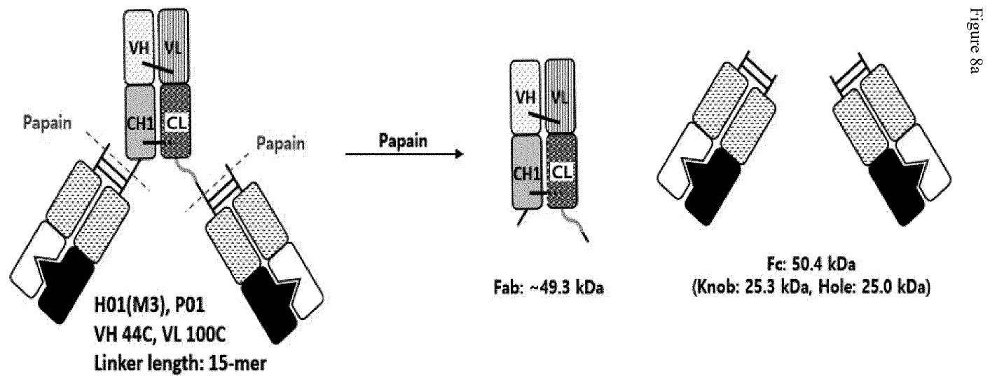

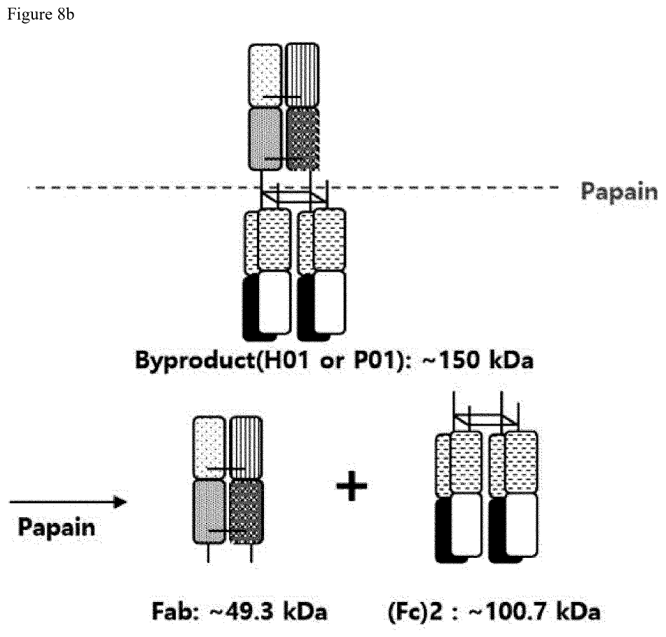

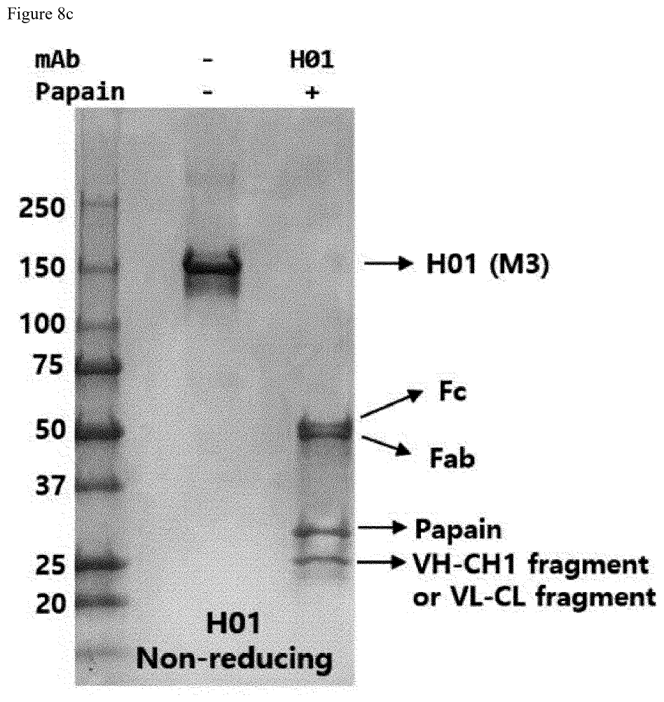

Papain recognizes specific sequences in the hinge region and induces antibody digestion. In the case of the Fab-(Fc)₂ structure, when papain digestion is performed, it is cleaved into a Fab portion of approximately 49.3 kDa and two Fc domains of approximately 50.4 kDa (FIG. 8a). However, if abnormal disulfide bonds are formed in the hinge region, unwanted inter-chain disulfide bond byproducts could be observed (FIG. 8b). In this case, a Fab fragment of approximately 49.3 kDa and an abnormal (Fc)₂ product of approximately 100.7 kDa could be observed (FIG. 8b).

To verify this, papain digestion of H01 and P01 was performed. Papain (Sigma, P3125) was used by diluting it to 0.1 mg/mL in digestion buffer (20 mM EDTA+10 mM Cys-HCl in PBS pH 7.4). 200 μg of H01 and P01 were digested at 37° C. for 2 hours, and then SDS-PAGE was performed. As a result of SDS-PAGE performed under non-reducing conditions, abnormal (Fc)₂ at about 100 kDa was not identified (FIGS. 8c and 8d).

### Example 4. Analysis of Physical Properties of H01 wt

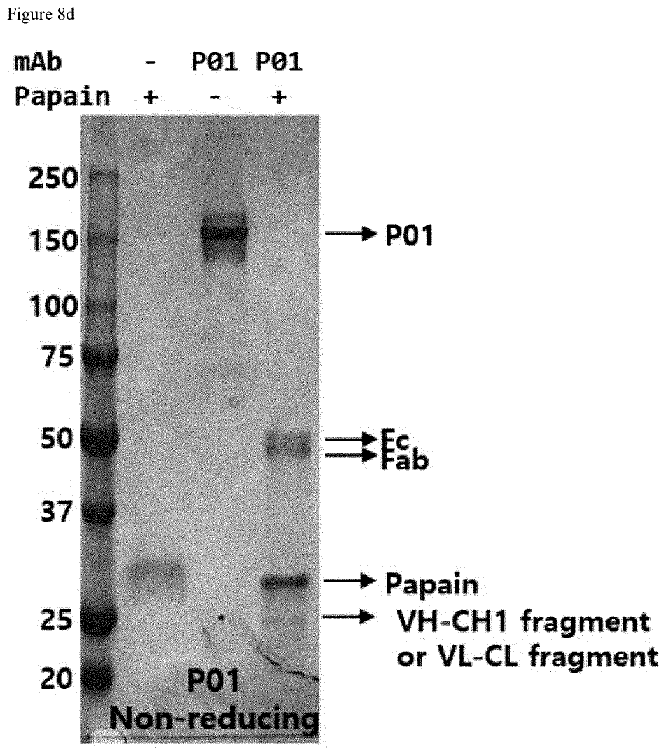

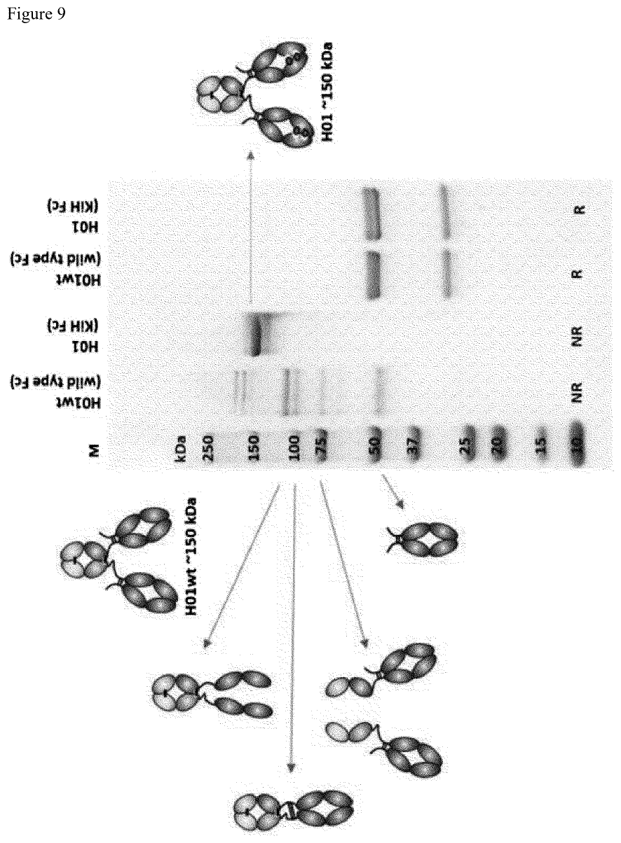

In H01, four Fc monomers are assembled into two Fc dimers due to knob-into-hole mutations, resulting in a structure as shown in FIG. 6a. To analyze the effect of the knob-into-hole mutations on the formation of H01 structure, the Fc hole monomer polypeptide (SEQ ID NO: 7) was substituted with a polypeptide (SEQ ID NO: 39) corresponding to the wild type IgG1 Fc monomer (Table 17). The two knob polypeptides (SEQ ID NOs: 14 and 15) constituting H01 were also substituted with polypeptides (SEQ ID NOs: 40 and 41) corresponding to the wild type IgG1 Fc monomer (Table 17). This novel antibody format consisting of two wtFc polypeptides (SEQ ID NO: 39), one TraH-G44C-wtFc polypeptide (SEQ ID NO: 40), and one TraL-Q100C-wtFc polypeptide (SEQ ID NO: 41) is referred to as H01 wt (FIG. 9).

Expression vectors containing sequences corresponding to wtFc (SEQ ID NO: 39), TraH-G44C-wtFc (SEQ ID NO: 40), and TraL-Q100C-wtFc (SEQ ID NO: 41) were co-transfected into EXPICHO-S™ (Gibco, A29127), and purification and analysis were performed in the same manner as described in Example 1. SDS-PAGE analysis under non-reducing conditions (NR) identified a small amount of H01 wt at about 150 kDa, and most of H01 wt were expressed as abnormally structured byproducts (FIG. 9). In the case of H01, which has knob-into-hole mutations in the Fc region, each polypeptide was efficiently assembled into a product that is identified at about 150 kDa (FIG. 9).

TABLE 17

| Name | Sequence | SEQ ID NO |
|---|---|---|
| wtFc | DKTHTCPPCPAPELLGGPSVFLFPPKPKDTLMISRTPEVTCVVVDVSHE | 39 |
| | DPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGK | |
| TraH- | EVQLVESGGGLVQPGGSLRLSCAASGFNIKDTYIHWVRQAPGKCLEW | 40 |
| G44C- | VARIYPTNGYTRYADSVKGRFTISADTSKNTAYLQMNSLRAEDTAVY | |
| wtFc | YCSRWGGDGFYAMDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQG | |
| | NVFSCSVMHEALHNHYTQKSLSLSPGK | |
| TraL- | DIQMTQSPSSLSASVGDRVTITCRASQDVNTAVAWYQQKPGKAPKLL | 41 |
| Q100C- | IYSASFLYSGVPSRFSGSRSGTDFTLTISSLQPEDFATYYCQQHYTTPPT | |
| wtFc | FGCGTKVEIKRTVAAPSVFIFPPSDEQLKSGTASVVCLLNNFYPREAKV | |
| | QWKVDNALQSGNSQESVTEQDSKDSTYSLSSTLTLSKADYEKHKVY | |
| | ACEVTHQGLSSPVTKSFNRGECGGGGSGGGGSGGGGSEPKSSDKTHT | |
| | CPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKS | |
| | RWQQGNVFSCSVMHEALHNHYTQKSLSLSPGK | |

Table 18 below shows the nucleotide sequences encoding wtFc, TraH-G44C-wtFc, and TraL-Q100C-wtFc of H011 wt.

TABLE 18

| Name | Sequence | SEQ ID NO |
|---|---|---|
| wtFc | GACAAAACTCACACATGCCCACCGTGCCCAGCACCTGAACTCCTG | 476 |
| | GGGGGACCGTCAGTCTTCCTCTTCCCCCCAAAACCCAAGGACACCC | |
| | TCATGATCTCCCGGACCCCTGAGGTCACATGCGTGGTGGTGGACGT | |
| | GAGCCACGAAGACCCTGAGGTCAAGTTCAACTGGTACGTGGACGG | |
| | CGTGGAGGTGCATAATGCCAAGACAAAGCCGCGGGAGGAGCAGT | |
| | ACAACAGCACGTACCGTGTGGTCAGCGTCCTCACCGTCCTGCACCA | |
| | GGACTGGCTGAATGGCAAGGAGTACAAGTGCAAGGTCTCCAACAA | |
| | AGCCCTCCCAGCCCCCATCGAGAAAACCATCTCCAAAGCCAAAGG | |
| | GCAGCCCCGAGAACCACAGGTGTACACCCTGCCCCCATCCCGGGA | |
| | GGAGATGACCAAGAACCAGGTCAGCCTGACGTGCCTGGTCAAAGG | |
| | CTTCTATCCCAGCGACATCGCCGTGGAGTGGGAGAGCAATGGGCA | |
| | GCCGGAGAACAACTACAAGACCACGCCTCCCGTGCTGGACTCCGA | |
| | CGGCTCCTTCTTCCTCTACAGCAAGCTCACCGTGGACAAGAGCAGG | |
| | TGGCAGCAGGGGAACGTCTTCTCATGCTCCGTGATGCATGAGGCTC | |
| | TGCACAACCACTACACGCAGAAGAGCCTCTCCCTGTCTCCGGGTAA | |
| | A | |
| TraH- | GAAGTTCAACTTGTGGAGTCTGGAGGCGGTTTGGTACAACCTGGA | 477 |
| G44C- | GGTTCACTTCGTTTGTCATGTGCTGCCTCTGGGTTTAATATCAAGG | |
| wtFc | ATACTTATATTCATTGGGTTCGCCAGGCACCCGGAAAATGCCTGGA | |
| | ATGGGTGGCCCGTATATATCCCACTAACGGTTACACTCGATATGCT | |
| | GACTCTGTAAAGGGTCGATTTACTATATCCGCTGATACTAGCAAAA | |
| | ACACTGCCTACCTCCAGATGAACAGTCTGCGCGCCGAGGATACAG | |
| | CAGTTTATTATTGTTCCCGATGGGGAGGTGACGGGTTCTATGCCAT | |
| | GGACTACTGGGGTCAAGGGACACTGGTAACCGTTTCTTCTGCTAGC | |
| | ACCAAAGGACCTAGTGTTTTTCCTCTTGCCCCTTCCTCAAAGTCTAC | |
| | CTCTGGGGGGACAGCCGCTCTGGGCTGCCTGGTCAAGGATTATTTC | |
| | CCAGAGCCTGTCACTGTCAGTTGGAACTCTGGAGCCTTGACTTCTG | |
| | GTGTTCATACATTTCCTGCTGTCCTTCAAAGCAGCGGCTTGTACTC | |
| | ATTGTCTTCTGTTGTGACAGTACCCTCAAGCAGCCTCGGCACTCAG | |
| | ACATACATCTGCAATGTCAACCACAAACCCTCAAATACAAAGGTA | |
| | GATAAAAAAGTCGAACCAAAGTCTTGTGACAAAACTCACACGTGC | |
| | CCACCGTGCCCAGCACCTGAACTCCTGGGGGGACCGTCAGTCTTCC | |
| | TCTTCCCCCCAAAACCCAAGGACACCCTCATGATCTCCCGGACCCC | |
| | TGAGGTCACATGCGTGGTGGTGGACGTGAGCCACGAAGACCCTGA | |
| | GGTCAAGTTCAACTGGTACGTGGACGGCGTGGAGGTGCATAATGC | |
| | CAAGACAAAGCCGCGGGAGGAGCAGTACAACAGCACGTACCGTGT | |
| | GGTCAGCGTCCTCACCGTCCTGCACCAGGACTGGCTGAATGGCAA | |
| | GGAGTACAAGTGCAAGGTCTCCAACAAAGCCCTCCCAGCCCCCAT | |
| | CGAGAAAACCATCTCCAAAGCCAAAGGGCAGCCCCGAGAACCACA | |
| | GGTGTACACCCTGCCCCCAAGCCGGGAGGAGATGACCAAGAACCA | |
| | GGTCAGCCTGACGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACACG | |
| | CAGAAGAGCCTCTCCCTGTCTCCGGGTAAA | |
| TraL- | GACATTCAGATGACCCAATCCCCCTCTAGCCTTTCAGCCTCCGTAG | 478 |
| Q100C- | GGGACAGAGTTACAATAACCTGTAGGGCTAGTCAGGACGTTAATA | |
| wtFc | CAGCAGTCGCATGGTACCAGCAGAAGCCCGGTAAGGCTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGATGTGGCACAAAAGTAGAGATTAA | |
| | GCGTACGGTGGCAGCTCCCAGCGTTTTTATCTTTCCCCCATCCGAC | |
| | GAGCAGCTCAAGAGTGGCACTGCCTCTGTAGTTTGTTTGCTGAATA | |
| | ACTTCTATCCACGTGAAGCAAAAGTACAGTGGAAGGTCGATAATG | |
| | CCCTTCAGAGCGGTAACAGCCAAGAAAGTGTTACCGAGCAAGATT | |
| | CCAAAGATTCCACTTACAGTCTGTCCAGCACATTGACACTGAGTAA | |
| | GGCTGATTACGAAAAACACAAGGTGTACGCATGCGAGGTGACACA | |
| | CCAAGGTCTTTCATCTCCTGTAACTAAGAGCTTTAACCGGGGAGAA | |
| | TGTGGTGGTGGGGGCAGCGGGGGCGGAGGTAGTGGAGGCGGCGG | |
| | TAGTGAACCAAAGAGTAGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAAG | |
| | TTCAACTGGTACGTGGACGGCGTGGAGGTGCATAATGCCAAGACA | |
| | AAGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAAA | |
| | ACCATCTCCAAAGCCAAAGGGCAGCCCCGAGAACCACAGGTGTAC | |
| | ACCCTGCCCCCAAGCCGGGAGGAGATGACCAAGAACCAGGTCAGC | |
| | CTGACGTGCCTGGTCAAAGGCTTCTATCCCAGCGACATCGCCGTGG | |
| | AGTGGGAGAGCAATGGGCAGCCGGAGAACAACTACAAGACCACG | |
| | CCTCCCGTGCTGGACTCCGACGGCTCCTTCTTCCTCTACAGCAAGC | |
| | TCACCGTGGACAAGAGCAGGTGGCAGCAGGGGAACGTCTTCTCAT | |
| | GCTCCGTGATGCATGAGGCTCTGCACAACCACTACACGCAGAAGA | |
| | GCCTCTCCCTGTCTCCGGGTAAA | |

### Example 5. Characterization of H01Fv Variant

The schematic diagram of Fv-(Fc)₂, in which two Fc domains are fused in parallel to an antibody Fv fragment, is shown in FIGS. 10a to 10g. Fv consists of a VH domain and a VL domain. In order to improve interaction of the domains at the domain interfaces, a disulfide bond was formed artificially by substituting an amino acid at a specific position with cysteine (FIGS. 10a to 10h, Table 19).

TABLE 19

| | VH Mutation site | VL Mutation site | MW |
|---|---|---|---|
| | (Kabat numbering) | (Kabat numbering) | (kDa) |
| H01Fv1 | G44C | Q100C | 128.21 |
| H01Fv2 | Q105C | A43C | 128.19 |
| H01Fv3 | F122C | S121C | 130.98 |
| H01Fv4 | G44C, Q105C | Q100C, A43C | 130.01 |
| H01Fv5 | G44C, F122C | Q100C, S121C | 130.00 |
| H01Fv6 | Q105C, F126C | A43C, S121C | 130.99 |
| H01Fv7 | G44C, Q105C, F126C | Q100C, A43C, S121C | 131.01 |

Table 20 shows the polypeptide sequences constituting H01Fv1 to H01Fv7.

TABLE 20

| | | SEQ |
|---|---|---|
| Name | Sequence | ID NO |
| Fc-Hole-RF | DKTHTCPPCPAPELLGGPSVFLFPPKPKDTLMISRTPEVTCVVVDVSHE | 789 |
| | DPEVKFNWYVDGVEVHNAKTKPREEQYNSTYRVVSVLTVLHQDWL | |
| | NGKEYKCKVSNKALPAPIEKTISKAKGQPREPQVCTLPPSREEMTKNQ | |
| | VSLSCAVKGFYPSDIAVEWESNGQPENNYKTTPPVLDSDGSFFLVSKL | |
| | TVDKSRWQQGNVFSCSVMHEALHNRFTQKSLSLSPGK | |
| H01Fv1-HC | EVQLVESGGGLVQPGGSLRLSCAASGFNIKDTYIHWVRQAPGKCLEW | 790 |
| | VARIYPTNGYTRYADSVKGRFTISADTSKNTAYLQMNSLRAEDTAVY | |
| | YCSRWGGDGFYAMDYWGQGTLVTVSSEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPSD | |
| | IAVEWESNGQPENNYKTTPPVLDSDGSFFLYSKLTVDKSRWQQGNVF | |
| | SCSVMHEALHNHYTQKSLSLSPGK | |
| H01Fv1-LC | DIQMTQSPSSLSASVGDRVTITCRASQDVNTAVAWYQQKPGKAPKLL | 791 |
| | IYSASFLYSGVPSRFSGSRSGTDFTLTISSLQPEDFATYYCQQHYTTPPT | |
| | FGCGTKVEIKREPKSCDKTHTCPPCPAPELLGGPSVFLFPPKPKDTLMI | |
| | SRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREP | |
| | QVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQK | |
| | SLSLSPGK | |
| H01Fv2-HC | EVQLVESGGGLVQPGGSLRLSCAASGFNIKDTYIHWVRQAPGKGLEW | 792 |
| | VARIYPTNGYTRYADSVKGRFTISADTSKNTAYLQMNSLRAEDTAVY | |
| | YCSRWGGDGFYAMDYWGCGTLVTVSSEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPSD | |
| | IAVEWESNGQPENNYKTTPPVLDSDGSFFLYSKLTVDKSRWQQGNVF | |
| | SCSVMHEALHNHYTQKSLSLSPGK | |
| H01Fv2-LC | DIQMTQSPSSLSASVGDRVTITCRASQDVNTAVAWYQQKPGKCPKLLI | 793 |
| | YSASFLYSGVPSRFSGSRSGTDFTLTISSLQPEDFATYYCQQHYTTPPTF | |
| | GQGTKVEIKREPKSCDKTHTCPPCPAPELLGGPSVFLFPPKPKDTLMIS | |
| | RTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQ | |
| | VYTLPPCREEMTKNQVSLWCLVKGFYPSDIAVEWESNGQPENNYKTT | |
| | PPVLDSDGSFFLYSKLTVDKSRWQQGNVFSCSVMHEALHNHYTQKSL | |
| | SLSPGK | |
| H01Fv3-HC | EVQLVESGGGLVQPGGSLRLSCAASGFNIKDTYIHWVRQAPGKGLEW | 794 |
| | VARIYPTNGYTRYADSVKGRFTISADTSKNTAYLQMNSLRAEDTAVY | |
| | YCSRWGGDGFYAMDYWGQGTLVTVSSASTKGPSVCPLAPEPKSCDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPCREEMTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| H01Fv3-LC | DIQMTQSPSSLSASVGDRVTITCRASQDVNTAVAWYQQKPGKAPKLL | 795 |
| | IYSASFLYSGVPSRFSGSRSGTDFTLTISSLQPEDFATYYCQQHYTTPPT | |
| | FGQGTKVEIKRTVAAPSVFIFPPCGGGSEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPCREEMTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGK | |
| H01Fv4-HC | EVQLVESGGGLVQPGGSLRLSCAASGFNIKDTYIHWVRQAPGKCLEW | 796 |
| | VARIYPTNGYTRYADSVKGRFTISADTSKNTAYLQMNSLRAEDTAVY | |
| | YCSRWGGDGFYAMDYWGCGTLVTVSSASTKGPSVCPLAPEPKSCDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPCREEMTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| H01Fv4-LC | DIQMTQSPSSLSASVGDRVTITCRASQDVNTAVAWYQQKPGKCPKLLI | 797 |
| | YSASFLYSGVPSRFSGSRSGTDFTLTISSLQPEDFATYYCQQHYTTPPTF | |
| | GCGTKVEIKREPKSCDKTHTCPPCPAPELLGGPSVFLFPPKPKDTLMIS | |
| | RTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQ | |
| | VYTLPPCREEMTKNQVSLWCLVKGFYPSDIAVEWESNGQPENNYKTT | |
| | PPVLDSDGSFFLYSKLTVDKSRWQQGNVFSCSVMHEALHNHYTQKSL | |
| | SLSPGK | |
| H01Fv5-HC | EVQLVESGGGLVQPGGSLRLSCAASGFNIKDTYIHWVRQAPGKCLEW | 798 |
| | VARIYPTNGYTRYADSVKGRFTISADTSKNTAYLQMNSLRAEDTAVY | |
| | YCSRWGGDGFYAMDYWGQGTLVTVSSASTKGPSVCPLAPEPKSCDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPCREEMTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| H01Fv5-LC | DIQMTQSPSSLSASVGDRVTITCRASQDVNTAVAWYQQKPGKAPKLL | 799 |
| | IYSASFLYSGVPSRFSGSRSGTDFTLTISSLQPEDFATYYCQQHYTTPPT | |
| | FGCGTKVEIKRTVAAPSVFIFPPCGGGSEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALP | |
| | APIEKTISKAKGQPREPQVYTLPPCREEMTKNQVSLWCLVKGFYPSDI | |
| | AVEWESNGQPENNYKTTPPVLDSDGSFFLYSKLTVDKSRWQQGNVFS | |
| | CSVMHEALHNHYTQKSLSLSPGK | |
| H01Fv6-HC | EVQLVESGGGLVQPGGSLRLSCAASGFNIKDTYIHWVRQAPGKGLEW | 800 |
| | VARIYPTNGYTRYADSVKGRFTISADTSKNTAYLQMNSLRAEDTAVY | |
| | YCSRWGGDGFYAMDYWGCGTLVTVSSASTKGPSVCPLAPEPKSCDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPCREEMTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| H01Fv6-LC | DIQMTQSPSSLSASVGDRVTITCRASQDVNTAVAWYQQKPGKCPKLLI | 801 |
| | YSASFLYSGVPSRFSGSRSGTDFTLTISSLQPEDFATYYCQQHYTTPPTF | |
| | GQGTKVEIKRTVAAPSVFIFPPCGGGSEPKSCDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPCREEMTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |
| H01Fv7-HC | EVQLVESGGGLVQPGGSLRLSCAASGFNIKDTYIHWVRQAPGKCLEW | 802 |
| | VARIYPTNGYTRYADSVKGRFTISADTSKNTAYLQMNSLRAEDTAVY | |
| | YCSRWGGDGFYAMDYWGCGTLVTVSSASTKGPSVCPLAPEPKSCDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNG | |
| | KEYKCKVSNKALPAPIEKTISKAKGQPREPQVYTLPPCREEMTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| H01Fv7-LC | DIQMTQSPSSLSASVGDRVTITCRASQDVNTAVAWYQQKPGKCPKLLI | 803 |
| | YSASFLYSGVPSRFSGSRSGTDFTLTISSLQPEDFATYYCQQHYTTPPTF | |
| | GCGTKVEIKRTVAAPSVFIFPPCGGGSEPKSCDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALPA | |
| | PIEKTISKAKGQPREPQVYTLPPCREEMTKNQVSLWCLVKGFYPSDIA | |
| | VEWESNGQPENNYKTTPPVLDSDGSFFLYSKLTVDKSRWQQGNVFSC | |
| | SVMHEALHNHYTQKSLSLSPGK | |

Table 21 shows the nucleotide sequences encoding polypeptides constituting H01Fv1 to H01Fv7.

TABLE 21

| | | SEQ |
|---|---|---|
| Name | Sequence | ID NO |
| Fc-Hole- | GACAAAACTCACACATGCCCACCGTGCCCAGCACCTGAACTCCTG | 804 |
| RF | GGGGGACCGTCAGTCTTCCTCTTCCCCCCAAAACCCAAGGACACCC | |
| | TCATGATCTCCCGGACCCCTGAGGTCACATGCGTGGTGGTGGACGT | |
| | GAGCCACGAAGACCCTGAGGTCAAGTTCAACTGGTACGTGGACGG | |
| | CGTGGAGGTGCATAATGCCAAGACAAAGCCGCGGGAGGAGCAGT | |
| | ACAACAGCACGTACCGTGTGGTCAGCGTCCTCACCGTCCTGCACCA | |
| | GGACTGGCTGAATGGCAAGGAGTACAAGTGCAAGGTCTCCAACAA | |
| | AGCCCTCCCAGCCCCCATCGAGAAAACCATCTCCAAAGCCAAAGG | |
| | GCAGCCCCGAGAACCACAGGTGTGCACCCTGCCCCCATCCCGGGA | |
| | GGAGATGACCAAGAACCAGGTCAGCCTGAGCTGCGCGGTCAAAGG | |
| | CTTCTATCCCAGCGACATCGCCGTGGAGTGGGAGAGCAATGGGCA | |
| | GCCGGAGAACAACTACAAGACCACGCCTCCCGTGCTGGACTCCGA | |
| | CGGCTCCTTCTTCCTCGTGAGCAAGCTCACCGTGGACAAGAGCAGG | |
| | TGGCAGCAGGGGAACGTCTTCTCATGCTCCGTGATGCATGAGGCTC | |
| | TGCACAACAGATTTACGCAGAAGAGCCTCTCCCTGTCTCCGGGTAA | |
| | A | |
| H01Fv1-HC | GAAGTTCAACTTGTGGAGTCTGGAGGCGGTTTGGTACAACCTGGA | 805 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGGA | |
| | ATGGGTGGCCCGTATATATCCCACTAACGGTTACACTCGATATGCT | |
| | GACTCTGTAAAGGGTCGATTTACTATATCCGCTGATACTAGCAAAA | |
| | ACACTGCCTACCTCCAGATGAACAGTCTGCGCGCCGAGGATACAG | |
| | CAGTTTATTATTGTTCCCGATGGGGAGGTGACGGGTTCTATGCCAT | |
| | GGACTACTGGGGTCAAGGGACACTGGTAACCGTTTCTTCTGAACCA | |
| | AAGTCTTGTGACAAAACTCACACGTGCCCACCGTGCCCAGCACCTG | |
| | AACTCCTGGGGGGACCGTCAGTCTTCCTCTTCCCCCCAAAACCCAA | |
| | GGACACCCTCATGATCTCCCGGACCCCTGAGGTCACATGCGTGGTG | |
| | GTGGACGTGAGCCACGAAGACCCTGAGGTCAAGTTCAACTGGTAC | |
| | GTGGACGGCGTGGAGGTGCATAATGCCAAGACAAAGCCGCGGGA | |
| | GGAGCAGTACAACAGCACGTACCGTGTGGTCAGCGTCCTCACCGT | |
| | CCTGCACCAGGACTGGCTGAATGGCAAGGAGTACAAGTGCAAGGT | |
| | CTCCAACAAAGCCCTCCCAGCCCCCATCGAGAAAACCATCTCCAA | |
| | AGCCAAAGGGCAGCCCCGAGAACCACAGGTGTACACCCTGCCCCC | |
| | ATGCCGGGAGGAGATGACCAAGAACCAGGTCAGCCTGTGGTGCCT | |
| | GGTCAAAGGCTTCTATCCCAGCGACATCGCCGTGGAGTGGGAGAG | |
| | CAATGGGCAGCCGGAGAACAACTACAAGACCACGCCTCCCGTGCT | |
| | GGACTCCGACGGCTCCTTCTTCCTCTACAGCAAGCTCACCGTGGAC | |
| | AAGAGCAGGTGGCAGCAGGGGAACGTCTTCTCATGCTCCGTGATG | |
| | CATGAGGCTCTGCACAACCACTACACGCAGAAGAGCCTCTCCCTGT | |
| | CTCCGGGTAAA | |
| H01Fv1-LC | GACATTCAGATGACCCAATCCCCCTCTAGCCTTTCAGCCTCCGTAG | 806 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGATGTGGCACAAAAGTAGAGATTAA | |
| | GCGTGAACCAAAGTCTTGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAAG | |
| | TTCAACTGGTACGTGGACGGCGTGGAGGTGCATAATGCCAAGACA | |
| | AAGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAAA | |
| | ACCATCTCCAAAGCCAAAGGGCAGCCCCGAGAACCACAGGTGTAC | |
| | ACCCTGCCCCCATGCCGGGAGGAGATGACCAAGAACCAGGTCAGC | |
| | CTGTGGTGCCTGGTCAAAGGCTTCTATCCCAGCGACATCGCCGTGG | |
| | AGTGGGAGAGCAATGGGCAGCCGGAGAACAACTACAAGACCACG | |
| | CCTCCCGTGCTGGACTCCGACGGCTCCTTCTTCCTCTACAGCAAGC | |
| | TCACCGTGGACAAGAGCAGGTGGCAGCAGGGGAACGTCTTCTCAT | |
| | GCTCCGTGATGCATGAGGCTCTGCACAACCACTACACGCAGAAGA | |
| | GCCTCTCCCTGTCTCCGGGTAAA | |
| H01Fv2-HC | GAAGTTCAACTTGTGGAGTCTGGAGGCGGTTTGGTACAACCTGGA | 807 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATGC | |
| | TGACTCTGTAAAGGGTCGATTTACTATATCCGCTGATACTAGCAAA | |
| | AACACTGCCTACCTCCAGATGAACAGTCTGCGCGCCGAGGATACA | |
| | GCAGTTTATTATTGTTCCCGATGGGGAGGTGACGGGTTCTATGCCA | |
| | TGGACTACTGGGGTTGTGGGACACTGGTAACCGTTTCTTCTGAACC | |
| | AAAGTCTTGTGACAAAACTCACACGTGCCCACCGTGCCCAGCACCT | |
| | GAACTCCTGGGGGGACCGTCAGTCTTCCTCTTCCCCCCAAAACCCA | |
| | AGGACACCCTCATGATCTCCCGGACCCCTGAGGTCACATGCGTGGT | |
| | GGTGGACGTGAGCCACGAAGACCCTGAGGTCAAGTTCAACTGGTA | |
| | CGTGGACGGCGTGGAGGTGCATAATGCCAAGACAAAGCCGCGGGA | |
| | GGAGCAGTACAACAGCACGTACCGTGTGGTCAGCGTCCTCACCGT | |
| | CCTGCACCAGGACTGGCTGAATGGCAAGGAGTACAAGTGCAAGGT | |
| | CTCCAACAAAGCCCTCCCAGCCCCCATCGAGAAAACCATCTCCAA | |
| | AGCCAAAGGGCAGCCCCGAGAACCACAGGTGTACACCCTGCCCCC | |
| | ATGCCGGGAGGAGATGACCAAGAACCAGGTCAGCCTGTGGTGCCT | |
| | GGTCAAAGGCTTCTATCCCAGCGACATCGCCGTGGAGTGGGAGAG | |
| | CAATGGGCAGCCGGAGAACAACTACAAGACCACGCCTCCCGTGCT | |
| | GGACTCCGACGGCTCCTTCTTCCTCTACAGCAAGCTCACCGTGGAC | |
| | AAGAGCAGGTGGCAGCAGGGGAACGTCTTCTCATGCTCCGTGATG | |
| | CATGAGGCTCTGCACAACCACTACACGCAGAAGAGCCTCTCCCTGT | |
| | CTCCGGGTAAA | |
| H01Fv2-LC | GACATTCAGATGACCCAATCCCCCTCTAGCCTTTCAGCCTCCGTAG | 808 |
| | GGGACAGAGTTACAATAACCTGTAGGGCTAGTCAGGACGTTAATA | |
| | CAGCAGTCGCATGGTACCAGCAGAAGCCCGGTAAGTGTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGACAGGGCACAAAAGTAGAGATTAA | |
| | GCGTGAACCAAAGTCTTGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAAG | |
| | TTCAACTGGTACGTGGACGGCGTGGAGGTGCATAATGCCAAGACA | |
| | AAGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAAA | |
| | ACCATCTCCAAAGCCAAAGGGCAGCCCCGAGAACCACAGGTGTAC | |
| | ACCCTGCCCCCATGCCGGGAGGAGATGACCAAGAACCAGGTCAGC | |
| | CTGTGGTGCCTGGTCAAAGGCTTCTATCCCAGCGACATCGCCGTGG | |
| | AGTGGGAGAGCAATGGGCAGCCGGAGAACAACTACAAGACCACG | |
| | CCTCCCGTGCTGGACTCCGACGGCTCCTTCTTCCTCTACAGCAAGC | |
| | TCACCGTGGACAAGAGCAGGTGGCAGCAGGGGAACGTCTTCTCAT | |
| | GCTCCGTGATGCATGAGGCTCTGCACAACCACTACACGCAGAAGA | |
| | GCCTCTCCCTGTCTCCGGGTAAA | |
| H01Fv3-HC | GAAGTTCAACTTGTGGAGTCTGGAGGCGGTTTGGTACAACCTGGA | 809 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATGC | |
| | TGACTCTGTAAAGGGTCGATTTACTATATCCGCTGATACTAGCAAA | |
| | AACACTGCCTACCTCCAGATGAACAGTCTGCGCGCCGAGGATACA | |
| | GCAGTTTATTATTGTTCCCGATGGGGAGGTGACGGGTTCTATGCCA | |
| | TGGACTACTGGGGTCAAGGGACACTGGTAACCGTTTCTTCTGCTAG | |
| | CACCAAAGGACCTAGTGTTTGTCCTCTTGCCCCTGAACCAAAGTCT | |
| | TGTGACAAAACTCACACGTGCCCACCGTGCCCAGCACCTGAACTCC | |
| | TGGGGGGACCGTCAGTCTTCCTCTTCCCCCCAAAACCCAAGGACAC | |
| | CCTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCAC | |
| | CAGGACTGGCTGAATGGCAAGGAGTACAAGTGCAAGGTCTCCAAC | |
| | AAAGCCCTCCCAGCCCCCATCGAGAAAACCATCTCCAAAGCCAAA | |
| | GGGCAGCCCCGAGAACCACAGGTGTACACCCTGCCCCCATGCCGG | |
| | GAGGAGATGACCAAGAACCAGGTCAGCCTGTGGTGCCTGGTCAAA | |
| | GGCTTCTATCCCAGCGACATCGCCGTGGAGTGGGAGAGCAATGGG | |
| | CAGCCGGAGAACAACTACAAGACCACGCCTCCCGTGCTGGACTCC | |
| | GACGGCTCCTTCTTCCTCTACAGCAAGCTCACCGTGGACAAGAGCA | |
| | GGTGGCAGCAGGGGAACGTCTTCTCATGCTCCGTGATGCATGAGG | |
| | CTCTGCACAACCACTACACGCAGAAGAGCCTCTCCCTGTCTCCGGG | |
| | TAAA | |
| H01Fv3-LC | GACATTCAGATGACCCAATCCCCCTCTAGCCTTTCAGCCTCCGTAG | 810 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGACAGGGCACAAAAGTAGAGATTAA | |
| | GCGTACGGTGGCAGCTCCCAGCGTTTTTATCTTTCCCCCATGTGGA | |
| | GGTGGAAGTGAACCAAAGTCTTGTGACAAAACTCACACGTGCCCA | |
| | CCGTGCCCAGCACCTGAACTCCTGGGGGGACCGTCAGTCTTCCTCT | |
| | TCCCCCCAAAACCCAAGGACACCCTCATGATCTCCCGGACCCCTGA | |
| | GGTCACATGCGTGGTGGTGGACGTGAGCCACGAAGACCCTGAGGT | |
| | CAAGTTCAACTGGTACGTGGACGGCGTGGAGGTGCATAATGCCAA | |
| | GACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGGT | |
| | GTACACCCTGCCCCCATGCCGGGAGGAGATGACCAAGAACCAGGT | |
| | CAGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTTC | |
| | TCATGCTCCGTGATGCATGAGGCTCTGCACAACCACTACACGCAGA | |
| | AGAGCCTCTCCCTGTCTCCGGGTAAA | |
| H01Fv4-HC | GAAGTTCAACTTGTGGAGTCTGGAGGCGGTTTGGTACAACCTGGA | 811 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGGA | |
| | ATGGGTGGCCCGTATATATCCCACTAACGGTTACACTCGATATGCT | |
| | GACTCTGTAAAGGGTCGATTTACTATATCCGCTGATACTAGCAAAA | |
| | ACACTGCCTACCTCCAGATGAACAGTCTGCGCGCCGAGGATACAG | |
| | CAGTTTATTATTGTTCCCGATGGGGAGGTGACGGGTTCTATGCCAT | |
| | GGACTACTGGGGTTGTGGGACACTGGTAACCGTTTCTTCTGCTAGC | |
| | ACCAAAGGACCTAGTGTTTGTCCTCTTGCCCCTGAACCAAAGTCTT | |
| | GTGACAAAACTCACACGTGCCCACCGTGCCCAGCACCTGAACTCCT | |
| | GGGGGGACCGTCAGTCTTCCTCTTCCCCCCAAAACCCAAGGACACC | |
| | CTCATGATCTCCCGGACCCCTGAGGTCACATGCGTGGTGGTGGACG | |
| | TGAGCCACGAAGACCCTGAGGTCAAGTTCAACTGGTACGTGGACG | |
| | GCGTGGAGGTGCATAATGCCAAGACAAAGCCGCGGGAGGAGCAG | |
| | TACAACAGCACGTACCGTGTGGTCAGCGTCCTCACCGTCCTGCACC | |
| | AGGACTGGCTGAATGGCAAGGAGTACAAGTGCAAGGTCTCCAACA | |
| | AAGCCCTCCCAGCCCCCATCGAGAAAACCATCTCCAAAGCCAAAG | |
| | GGCAGCCCCGAGAACCACAGGTGTACACCCTGCCCCCATGCCGGG | |
| | AGGAGATGACCAAGAACCAGGTCAGCCTGTGGTGCCTGGTCAAAG | |
| | GCTTCTATCCCAGCGACATCGCCGTGGAGTGGGAGAGCAATGGGC | |
| | AGCCGGAGAACAACTACAAGACCACGCCTCCCGTGCTGGACTCCG | |
| | ACGGCTCCTTCTTCCTCTACAGCAAGCTCACCGTGGACAAGAGCAG | |
| | GTGGCAGCAGGGGAACGTCTTCTCATGCTCCGTGATGCATGAGGCT | |
| | CTGCACAACCACTACACGCAGAAGAGCCTCTCCCTGTCTCCGGGTA | |
| | AA | |
| H01Fv4-LC | GACATTCAGATGACCCAATCCCCCTCTAGCCTTTCAGCCTCCGTAG | 812 |
| | GGGACAGAGTTACAATAACCTGTAGGGCTAGTCAGGACGTTAATA | |
| | CAGCAGTCGCATGGTACCAGCAGAAGCCCGGTAAGTGTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGATGTGGCACAAAAGTAGAGATTAA | |
| | GCGTGAACCAAAGTCTTGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAAG | |
| | TTCAACTGGTACGTGGACGGCGTGGAGGTGCATAATGCCAAGACA | |
| | AAGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAAA | |
| | ACCATCTCCAAAGCCAAAGGGCAGCCCCGAGAACCACAGGTGTAC | |
| | ACCCTGCCCCCATGCCGGGAGGAGATGACCAAGAACCAGGTCAGC | |
| | CTGTGGTGCCTGGTCAAAGGCTTCTATCCCAGCGACATCGCCGTGG | |
| | AGTGGGAGAGCAATGGGCAGCCGGAGAACAACTACAAGACCACG | |
| | CCTCCCGTGCTGGACTCCGACGGCTCCTTCTTCCTCTACAGCAAGC | |
| | TCACCGTGGACAAGAGCAGGTGGCAGCAGGGGAACGTCTTCTCAT | |
| | GCTCCGTGATGCATGAGGCTCTGCACAACCACTACACGCAGAAGA | |
| | GCCTCTCCCTGTCTCCGGGTAAA | |
| H01Fv5-HC | GAAGTTCAACTTGTGGAGTCTGGAGGCGGTTTGGTACAACCTGGA | 813 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGGA | |
| | ATGGGTGGCCCGTATATATCCCACTAACGGTTACACTCGATATGCT | |
| | GACTCTGTAAAGGGTCGATTTACTATATCCGCTGATACTAGCAAAA | |
| | ACACTGCCTACCTCCAGATGAACAGTCTGCGCGCCGAGGATACAG | |
| | CAGTTTATTATTGTTCCCGATGGGGAGGTGACGGGTTCTATGCCAT | |
| | GGACTACTGGGGTCAAGGGACACTGGTAACCGTTTCTTCTGCTAGC | |
| | ACCAAAGGACCTAGTGTTTGTCCTCTTGCCCCTGAACCAAAGTCTT | |
| | GTGACAAAACTCACACGTGCCCACCGTGCCCAGCACCTGAACTCCT | |
| | GGGGGGACCGTCAGTCTTCCTCTTCCCCCCAAAACCCAAGGACACC | |
| | CTCATGATCTCCCGGACCCCTGAGGTCACATGCGTGGTGGTGGACG | |
| | TGAGCCACGAAGACCCTGAGGTCAAGTTCAACTGGTACGTGGACG | |
| | GCGTGGAGGTGCATAATGCCAAGACAAAGCCGCGGGAGGAGCAG | |
| | TACAACAGCACGTACCGTGTGGTCAGCGTCCTCACCGTCCTGCACC | |
| | AGGACTGGCTGAATGGCAAGGAGTACAAGTGCAAGGTCTCCAACA | |
| | AAGCCCTCCCAGCCCCCATCGAGAAAACCATCTCCAAAGCCAAAG | |
| | GGCAGCCCCGAGAACCACAGGTGTACACCCTGCCCCCATGCCGGG | |
| | AGGAGATGACCAAGAACCAGGTCAGCCTGTGGTGCCTGGTCAAAG | |
| | GCTTCTATCCCAGCGACATCGCCGTGGAGTGGGAGAGCAATGGGC | |
| | AGCCGGAGAACAACTACAAGACCACGCCTCCCGTGCTGGACTCCG | |
| | ACGGCTCCTTCTTCCTCTACAGCAAGCTCACCGTGGACAAGAGCAG | |
| | GTGGCAGCAGGGGAACGTCTTCTCATGCTCCGTGATGCATGAGGCT | |
| | CTGCACAACCACTACACGCAGAAGAGCCTCTCCCTGTCTCCGGGTA | |
| | AA | |
| H01Fv5-LC | GACATTCAGATGACCCAATCCCCCTCTAGCCTTTCAGCCTCCGTAG | 814 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGATGTGGCACAAAAGTAGAGATTAA | |
| | GCGTACGGTGGCAGCTCCCAGCGTTTTTATCTTTCCCCCATGTGGA | |
| | GGTGGAAGTGAACCAAAGTCTTGTGACAAAACTCACACGTGCCCA | |
| | CCGTGCCCAGCACCTGAACTCCTGGGGGGACCGTCAGTCTTCCTCT | |
| | TCCCCCCAAAACCCAAGGACACCCTCATGATCTCCCGGACCCCTGA | |
| | GGTCACATGCGTGGTGGTGGACGTGAGCCACGAAGACCCTGAGGT | |
| | CAAGTTCAACTGGTACGTGGACGGCGTGGAGGTGCATAATGCCAA | |
| | GACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGGT | |
| | GTACACCCTGCCCCCATGCCGGGAGGAGATGACCAAGAACCAGGT | |
| | CAGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTTC | |
| | TCATGCTCCGTGATGCATGAGGCTCTGCACAACCACTACACGCAGA | |
| | AGAGCCTCTCCCTGTCTCCGGGTAAA | |
| H01Fv6-HC | GAAGTTCAACTTGTGGAGTCTGGAGGCGGTTTGGTACAACCTGGA | 815 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATGC | |
| | TGACTCTGTAAAGGGTCGATTTACTATATCCGCTGATACTAGCAAA | |
| | AACACTGCCTACCTCCAGATGAACAGTCTGCGCGCCGAGGATACA | |
| | GCAGTTTATTATTGTTCCCGATGGGGAGGTGACGGGTTCTATGCCA | |
| | TGGACTACTGGGGTTGTGGGACACTGGTAACCGTTTCTTCTGCTAG | |
| | CACCAAAGGACCTAGTGTTTGTCCTCTTGCCCCTGAACCAAAGTCT | |
| | TGTGACAAAACTCACACGTGCCCACCGTGCCCAGCACCTGAACTCC | |
| | TGGGGGGACCGTCAGTCTTCCTCTTCCCCCCAAAACCCAAGGACAC | |
| | CCTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCAC | |
| | CAGGACTGGCTGAATGGCAAGGAGTACAAGTGCAAGGTCTCCAAC | |
| | AAAGCCCTCCCAGCCCCCATCGAGAAAACCATCTCCAAAGCCAAA | |
| | GGGCAGCCCCGAGAACCACAGGTGTACACCCTGCCCCCATGCCGG | |
| | GAGGAGATGACCAAGAACCAGGTCAGCCTGTGGTGCCTGGTCAAA | |
| | GGCTTCTATCCCAGCGACATCGCCGTGGAGTGGGAGAGCAATGGG | |
| | CAGCCGGAGAACAACTACAAGACCACGCCTCCCGTGCTGGACTCC | |
| | GACGGCTCCTTCTTCCTCTACAGCAAGCTCACCGTGGACAAGAGCA | |
| | GGTGGCAGCAGGGGAACGTCTTCTCATGCTCCGTGATGCATGAGG | |
| | CTCTGCACAACCACTACACGCAGAAGAGCCTCTCCCTGTCTCCGGG | |
| | TAAA | |
| H01Fv6-LC | GACATTCAGATGACCCAATCCCCCTCTAGCCTTTCAGCCTCCGTAG | 816 |
| | GGGACAGAGTTACAATAACCTGTAGGGCTAGTCAGGACGTTAATA | |
| | CAGCAGTCGCATGGTACCAGCAGAAGCCCGGTAAGTGTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGACAGGGCACAAAAGTAGAGATTAA | |
| | GCGTACGGTGGCAGCTCCCAGCGTTTTTATCTTTCCCCCATGTGGA | |
| | GGTGGAAGTGAACCAAAGTCTTGTGACAAAACTCACACGTGCCCA | |
| | CCGTGCCCAGCACCTGAACTCCTGGGGGGACCGTCAGTCTTCCTCT | |
| | TCCCCCCAAAACCCAAGGACACCCTCATGATCTCCCGGACCCCTGA | |
| | GGTCACATGCGTGGTGGTGGACGTGAGCCACGAAGACCCTGAGGT | |
| | CAAGTTCAACTGGTACGTGGACGGCGTGGAGGTGCATAATGCCAA | |
| | GACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGGT | |
| | GTACACCCTGCCCCCATGCCGGGAGGAGATGACCAAGAACCAGGT | |
| | CAGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTTC | |
| | TCATGCTCCGTGATGCATGAGGCTCTGCACAACCACTACACGCAGA | |
| | AGAGCCTCTCCCTGTCTCCGGGTAAA | |
| H01Fv7-HC | GAAGTTCAACTTGTGGAGTCTGGAGGCGGTTTGGTACAACCTGGA | 817 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGGA | |
| | ATGGGTGGCCCGTATATATCCCACTAACGGTTACACTCGATATGCT | |
| | GACTCTGTAAAGGGTCGATTTACTATATCCGCTGATACTAGCAAAA | |
| | ACACTGCCTACCTCCAGATGAACAGTCTGCGCGCCGAGGATACAG | |
| | CAGTTTATTATTGTTCCCGATGGGGAGGTGACGGGTTCTATGCCAT | |
| | GGACTACTGGGGTTGTGGGACACTGGTAACCGTTTCTTCTGCTAGC | |
| | ACCAAAGGACCTAGTGTTTGTCCTCTTGCCCCTGAACCAAAGTCTT | |
| | GTGACAAAACTCACACGTGCCCACCGTGCCCAGCACCTGAACTCCT | |
| | GGGGGGACCGTCAGTCTTCCTCTTCCCCCCAAAACCCAAGGACACC | |
| | CTCATGATCTCCCGGACCCCTGAGGTCACATGCGTGGTGGTGGACG | |
| | TGAGCCACGAAGACCCTGAGGTCAAGTTCAACTGGTACGTGGACG | |
| | GCGTGGAGGTGCATAATGCCAAGACAAAGCCGCGGGAGGAGCAG | |
| | TACAACAGCACGTACCGTGTGGTCAGCGTCCTCACCGTCCTGCACC | |
| | AGGACTGGCTGAATGGCAAGGAGTACAAGTGCAAGGTCTCCAACA | |
| | AAGCCCTCCCAGCCCCCATCGAGAAAACCATCTCCAAAGCCAAAG | |
| | GGCAGCCCCGAGAACCACAGGTGTACACCCTGCCCCCATGCCGGG | |
| | AGGAGATGACCAAGAACCAGGTCAGCCTGTGGTGCCTGGTCAAAG | |
| | GCTTCTATCCCAGCGACATCGCCGTGGAGTGGGAGAGCAATGGGC | |
| | AGCCGGAGAACAACTACAAGACCACGCCTCCCGTGCTGGACTCCG | |
| | ACGGCTCCTTCTTCCTCTACAGCAAGCTCACCGTGGACAAGAGCAG | |
| | GTGGCAGCAGGGGAACGTCTTCTCATGCTCCGTGATGCATGAGGCT | |
| | CTGCACAACCACTACACGCAGAAGAGCCTCTCCCTGTCTCCGGGTA | |
| | AA | |
| H01Fv7-LC | GACATTCAGATGACCCAATCCCCCTCTAGCCTTTCAGCCTCCGTAG | 818 |
| | GGGACAGAGTTACAATAACCTGTAGGGCTAGTCAGGACGTTAATA | |
| | CAGCAGTCGCATGGTACCAGCAGAAGCCCGGTAAGTGTCCTAAGC | |
| | TTTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACTA | |
| | CACTACTCCTCCAACCTTCGGATGTGGCACAAAAGTAGAGATTAA | |
| | GCGTACGGTGGCAGCTCCCAGCGTTTTTATCTTTCCCCCATGTGGA | |
| | GGTGGAAGTGAACCAAAGTCTTGTGACAAAACTCACACGTGCCCA | |
| | CCGTGCCCAGCACCTGAACTCCTGGGGGGACCGTCAGTCTTCCTCT | |
| | TCCCCCCAAAACCCAAGGACACCCTCATGATCTCCCGGACCCCTGA | |
| | GGTCACATGCGTGGTGGTGGACGTGAGCCACGAAGACCCTGAGGT | |
| | CAAGTTCAACTGGTACGTGGACGGCGTGGAGGTGCATAATGCCAA | |
| | GACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGGT | |
| | GTACACCCTGCCCCCATGCCGGGAGGAGATGACCAAGAACCAGGT | |
| | CAGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTTC | |
| | TCATGCTCCGTGATGCATGAGGCTCTGCACAACCACTACACGCAGA | |
| | AGAGCCTCTCCCTGTCTCCGGGTAAA | |

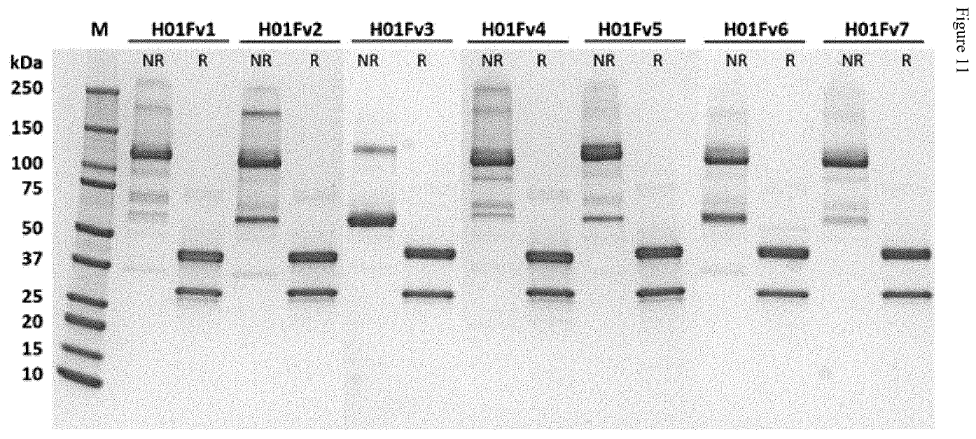

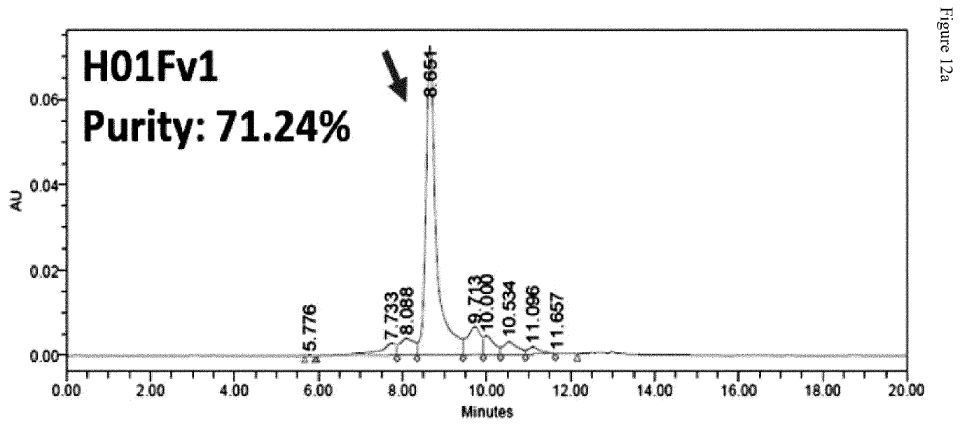

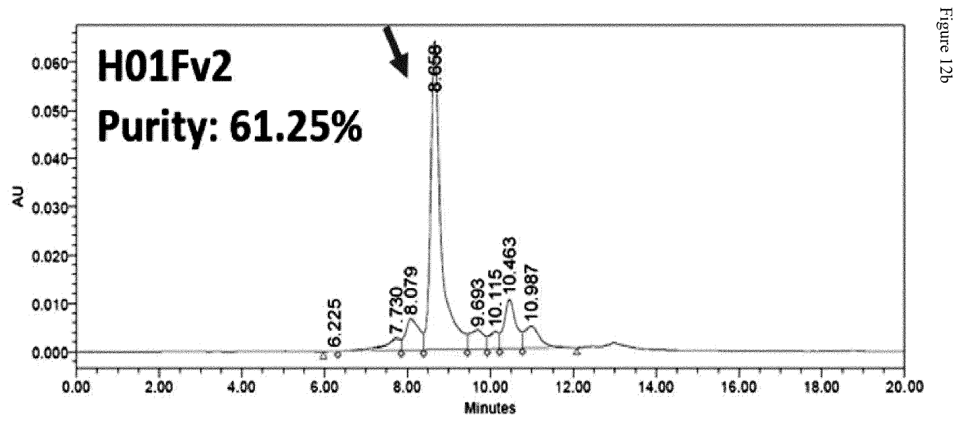

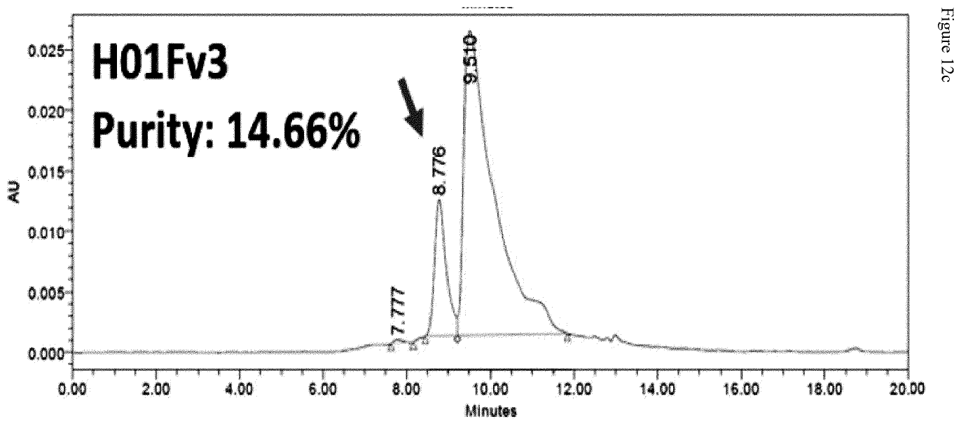

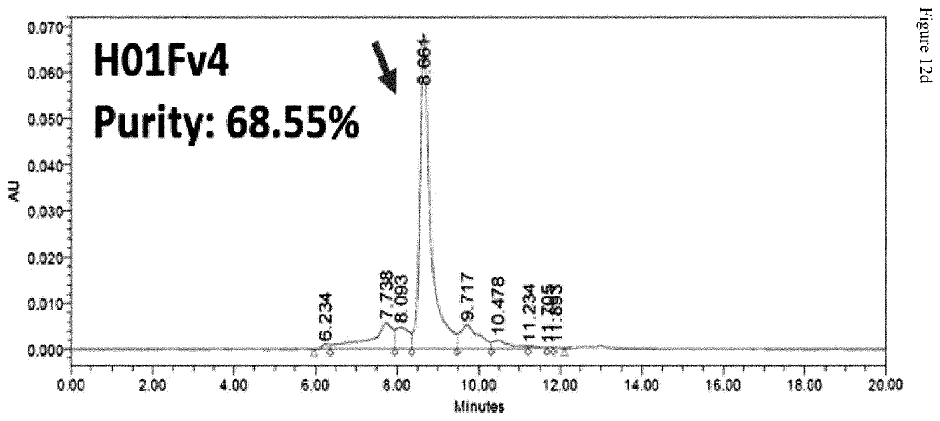

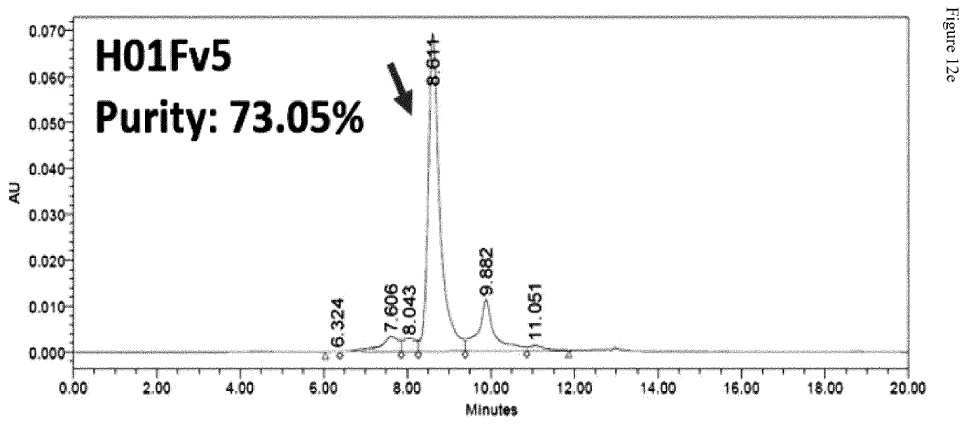

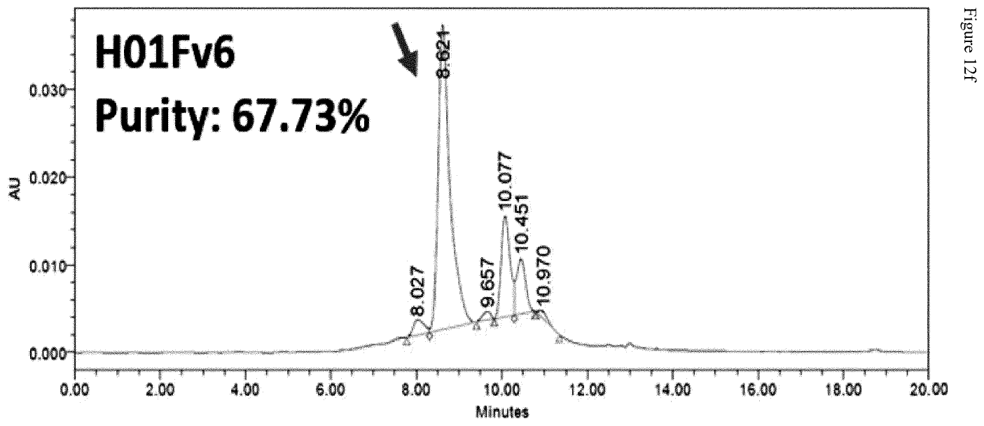

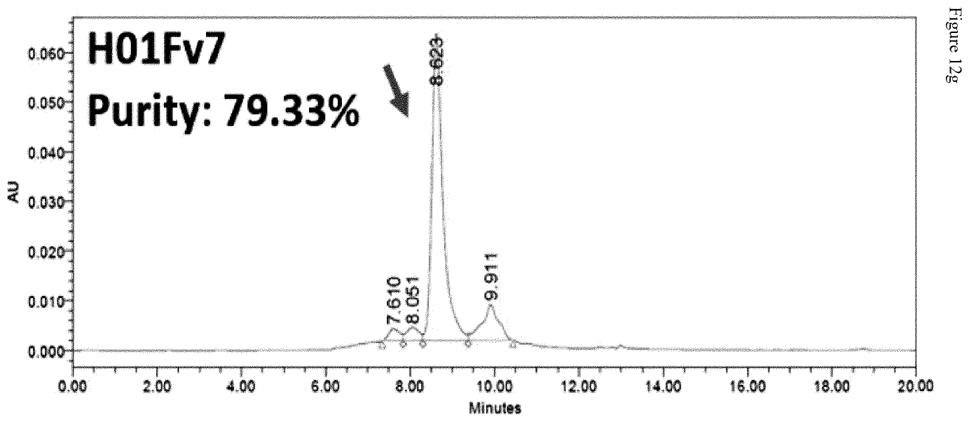

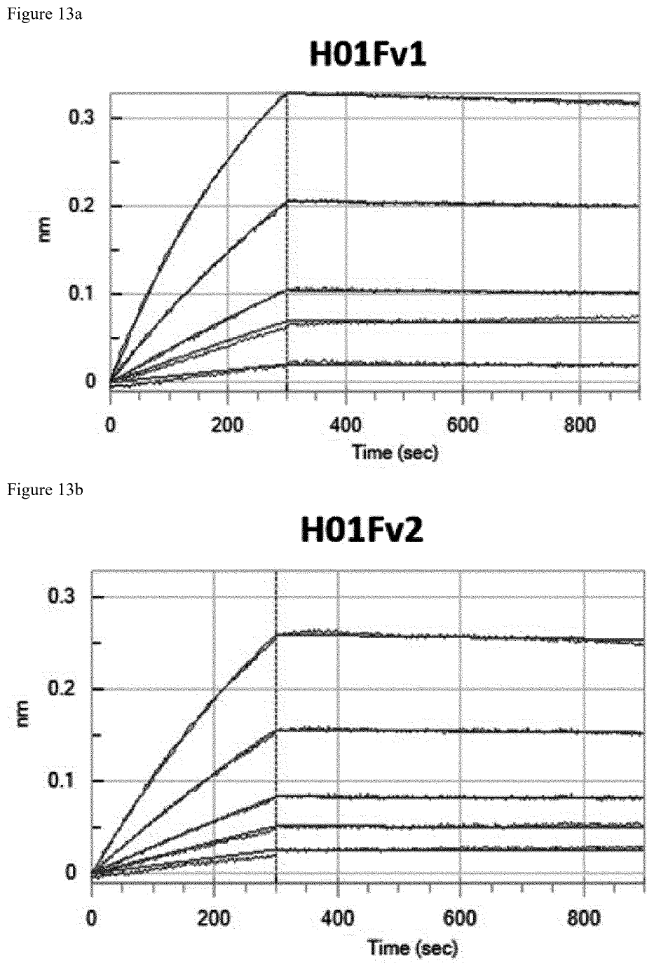

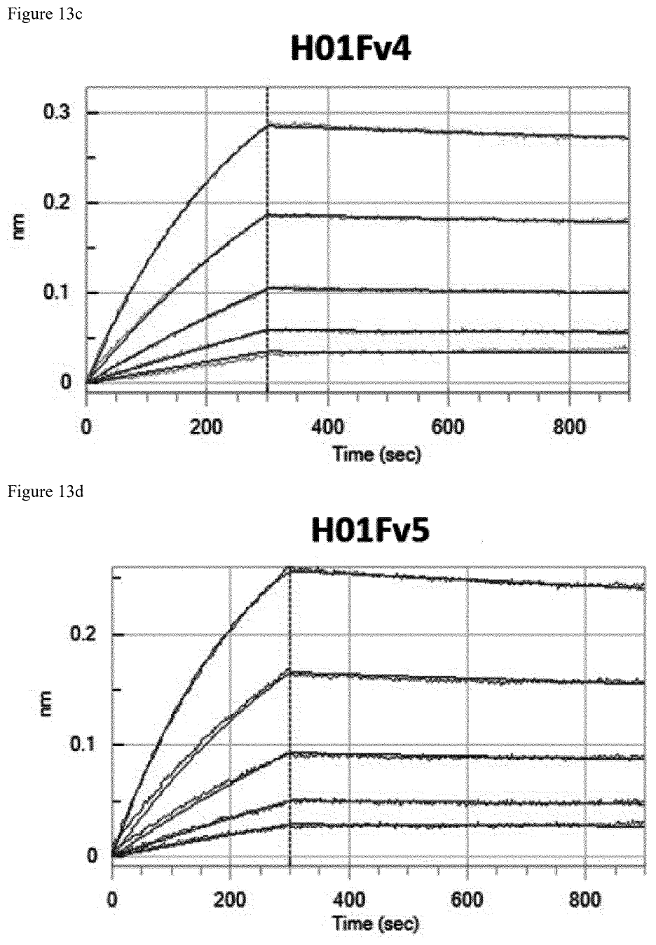

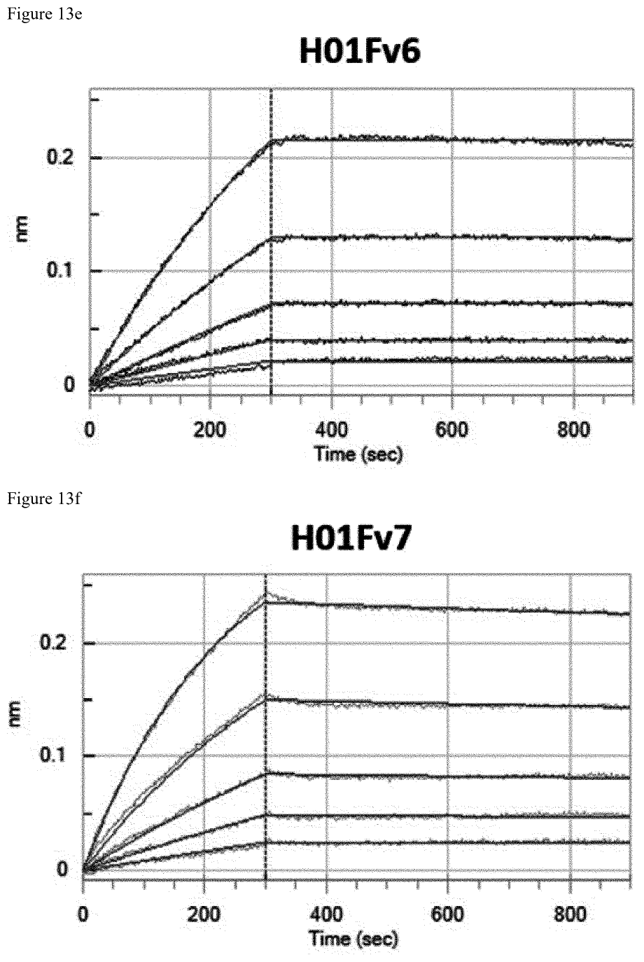

When expression vectors containing the sequences corresponding to Fc-Hole-F (SEQ ID NO: 789), H01Fv1-HC (SEQ ID NO: 790), and H01Fv1-LC (SEQ ID NO: 791) were co-transfect into EXPICHO-S™ (Gibco, A29127), H01Fv1 was formed (FIG. 10, Tables 20 and 21). Thereafter, it was purified through affinity chromatography using MABSELECT™ PrismA (Cytiva, 17549853). The Fc-Hole polypeptide (SEQ ID NO: 7) can form an Fc-Hole/Fc-Hole dimer. In order to remove this Fc-Hole/Fc-Hole dimer, H435R and Y436F mutations were introduced in the Fc-Hole polypeptide sequence (SEQ ID NO: 7) to produce the Fc-Hole-RF polypeptide (SEQ ID NO: 789) (FIG. 10, Tables 20 and 21). This prevents the Fc-Hole/Fc-Hole dimer from binding to Protein A resin and removes the mispaired Fc-Hole/Fc-Hole dimer (FIGS. 10 and 11). SDS-PAGE analysis identified the main product at about 130 kDa under non-reducing conditions (NR) (FIG. 11), and monomer purity was determined by SEC (FIG. 12). H01Fv3 exists mostly in an unpaired form of about 65 kDa, and the monomer purity of fully assembled form was determined to be 14.66% (FIGS. 11 and 12c). It was found that H01Fv1, H01Fv2, H01Fv4, H01Fv5, H01Fv6, and H01Fv7 have monomer purities of 71.24%, 61.25%, 68.55%, 73.05%, 67.73%, and 79.33%, respectively (FIG. 12). The binding characteristics of H01Fv1, H01Fv2, H01Fv4, H01Fv5, H01Fv6, and H01Fv7 were analyzed using Octet Red96e (Sartorius), a bio-layer interferometry (BLI) (FIG. 13). The human HER2 recombinant protein (R&D systems, 1129-ER) was loaded onto the Anti-Penta-HIS (HIS1K) biosensor (Sartorius, 18-5120), and then the binding constants of H01Fv1, H01Fv2, H01Fv4, H01Fv5, H01Fv6, and H01Fv7 were calculated (FIG. 13, Table 22).

TABLE 22

| Clone | KD(M) | kon(1/Ms) | kdis(1/s) |
|---|---|---|---|
| H01Fv1 | 2.44E−10 | 2.10E+05 | 5.11E−05 |
| H01Fv2 | 3.24E−10 | 1.17E+05 | 3.78E−05 |
| H01Fv3 | Not Determined | Not Determined | Not Determined |
| H01Fv4 | 3.26E−10 | 2.37E+05 | 7.71E−05 |
| H01Fv5 | 3.93E−10 | 2.73E+05 | 1.07E−04 |
| H01Fv6 | 4.87E−11 | 1.36E+05 | 6.61E−06 |
| H01Fv7 | 2.79E−10 | 2.76E+05 | 7.70E−05 |

### Example 6. Analysis of Thermal Stability

Analysis of thermal stability was performed using the PROTEIN THERMAL SHIFT™ Dye Kit (Applied biosystems, 4461146) according to the manufacturer's manual. Briefly, 5 μL of reaction buffer and 2.5 μL of dye included in the kit were mixed with 5 μg of trastuzumab, pertuzumab, H01, or P01, and the final volume was adjusted to 20 μL using PBS.

The mixture was incubated at 20° C. for 30 seconds in a C1000 thermal cycler (Bio-Rad, 1841000) equipped with a CFX96 optical reaction module (Bio-Rad, 1845096), and the fluorescence intensity of the plate was measured while increasing the temperature from 20° C. to 95° C. at 1° C./min, and the reaction was stopped after incubation at 95° C. for 30 seconds.

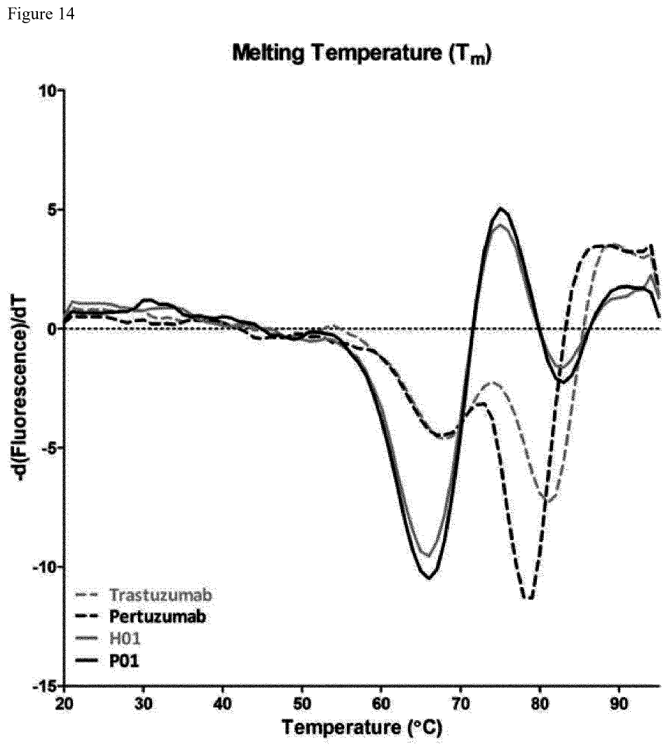

After the reaction, the median value of relative fluorescence unit (RFU) values was taken, and analysis of melting temperature (Tₘ) was performed. The Tₘ₁ values were found to be 68, 68, 66, and 66° C. and the Tₘ₂ values were found to be 81, 79, 83, and 83° C. for trastuzumab, pertuzumab, H01, and P01, respectively, indicating that H01 and P01 have Tₘ values similar to those of commercialized therapeutic antibodies (FIG. 14, Table 23).

TABLE 23

| Antibody | Tₘ₁ | Tₘ₂ |
|---|---|---|
| Trastuzumab | 68 | 81 |
| Pertuzumab | 68 | 79 |
| H01 | 66 | 83 |
| P01 | 66 | 83 |

### Example 7. Identification of Competitive Binding of H01 and P01

In order to identify whether H01 and P01 bind to different epitopes or compete for binding, Octet Red96e (Sartorius), a bio-layer interferometry (BLI), was used.

The human HER2 recombinant protein (R&D systems, 1129-ER) was loaded onto the Anti-Penta-HIS (HIS1K) biosensor (Sartorius, 18-5120). 100 nM human IgG1 (Bio X cell, BE0297) or 100 nM H01 or 100 nM trastuzumab was first bound to each biosensor loaded with HER2 antigen, followed by 100 nM human IgG1, 100 nM P01, or 100 nM pertuzumab to determine whether they bind competitively (FIG. 15). The binding signals (nm shift from baseline) measured at equilibrium after completion of Her2 recombinant protein loading were 0.620, 0.625, and 0.672 nm, respectively (FIG. 15, Table 24).

The first and second analytes were sequentially bound with binding time and dissociation time of 900 seconds. When the human IgG1 antibody was sequentially bound, it did not bind to HER2 (FIG. 15, Table 24). Sequential binding of H01 and P01 or sequential binding of trastuzumab and pertuzumab was observed, indicating that they bind to different epitopes (FIG. 15, Table 24). The binding signals (nm shift from baseline) of H01+P01 on the HER2-loaded sensor and the binding signals (nm shift from baseline) of trastuzumab+pertuzumab were measured to be 1.477 nm (=y-axis value at 4140 s−y-axis value at 1260 s), 0.923 nm (=y-axis value at 4140 s−y-axis value at 1260 s), respectively. The binding signals (nm shift from baseline) tend to increase as more proteins bind to the surface of the biosensor. Therefore, this indicates that to the same amount of HER2, H01 and P01 cause a greater amount of antibody binding than trastuzumab and pertuzumab (FIG. 15, Table 24).

TABLE 24

| | | HER2 | | |
|---|---|---|---|---|
| 1^{st} analyte - | | Loading | 1^{st} Analyte | 2^{nd} Analyte |
| 2^{nd} analyte | Baseline(0 s) | (1260 s) | binding(2160 s) | binding(4140 s) |
| hIgG1 - hIgG1 | 0 | 0.620 | 0.635 | 0.647 |
| H01- P01 | 0 | 0.625 | 1.407 | 2.102 |
| Trastuzumab - | 0 | 0.679 | 1.157 | 1.595 |
| Pertuzumab | | | | |

### Example 8. Quantification of Fc Loads

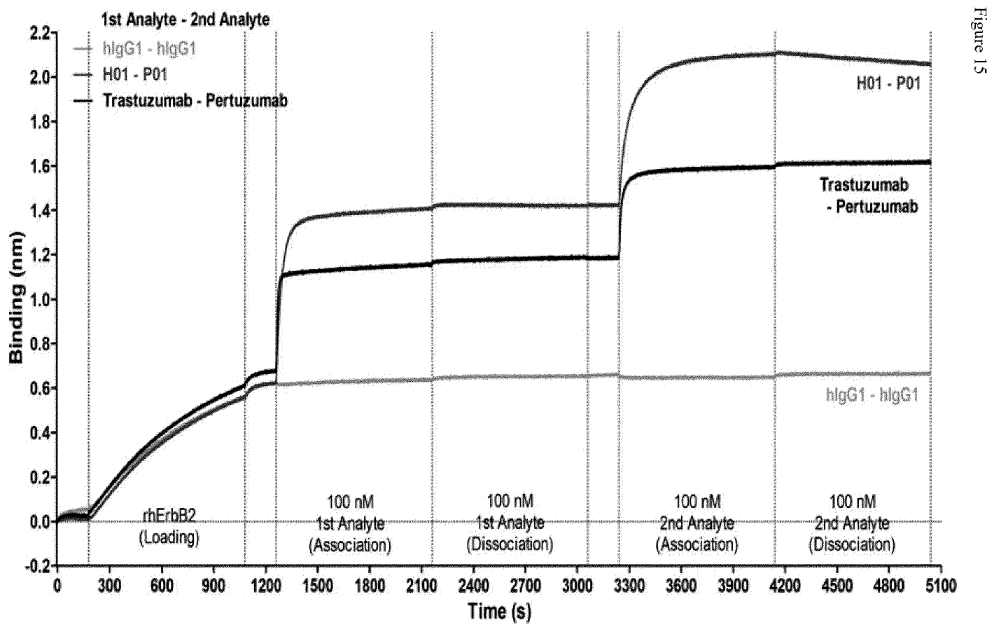

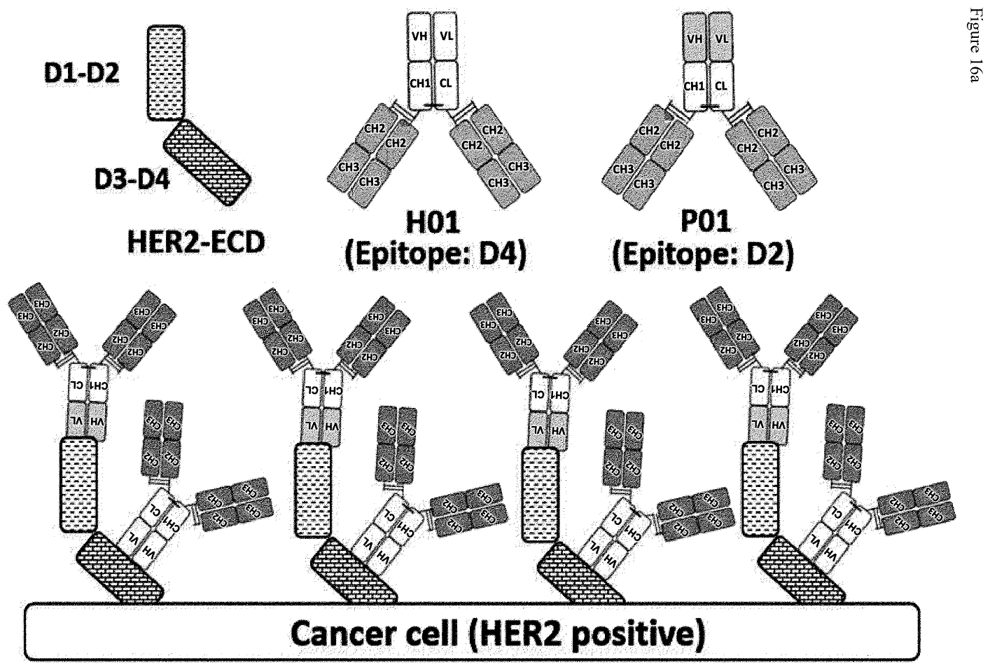

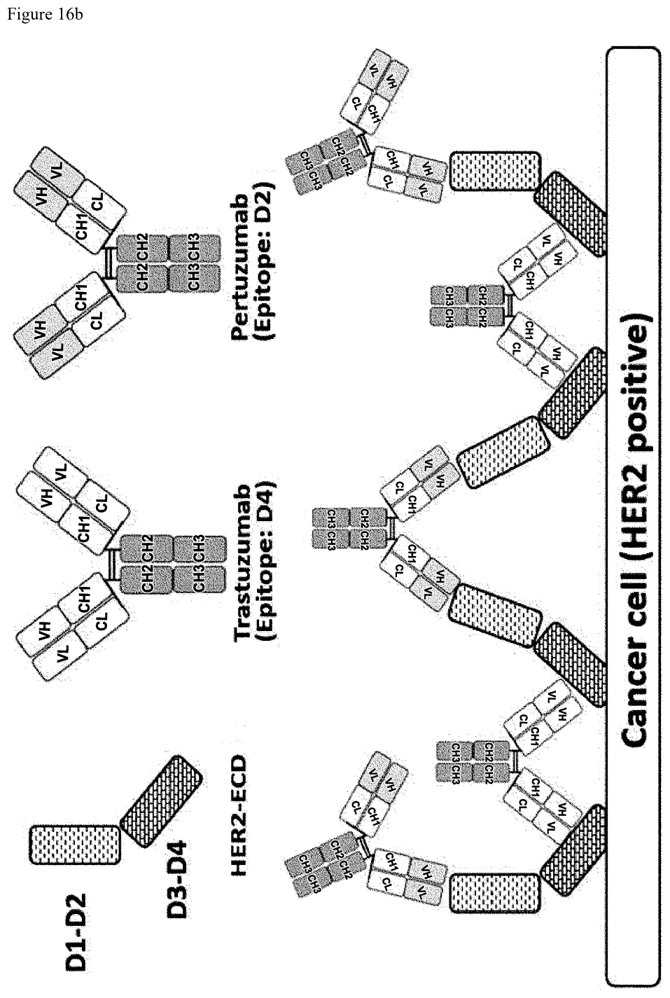

When H01 and P01 are treated in combination, a total of 16 Fc domains bind to four HER2 antigens present on the surface of cancer cells (FIG. 16a). When trastuzumab and pertuzumab are treated in combination, eight Fc domains bind to four HER2 antigens present on the surface of cancer cells if the binding is in monovalent mode, and fewer Fc domains can be bound if the binding is in bivalent mode (FIG. 16b). The combination of H01 and P01 should result in increased Fc loads on the surface of HER2-positive cancer cells than the combination of trastuzumab and pertuzumab and thereby should lead to a stronger effector function.

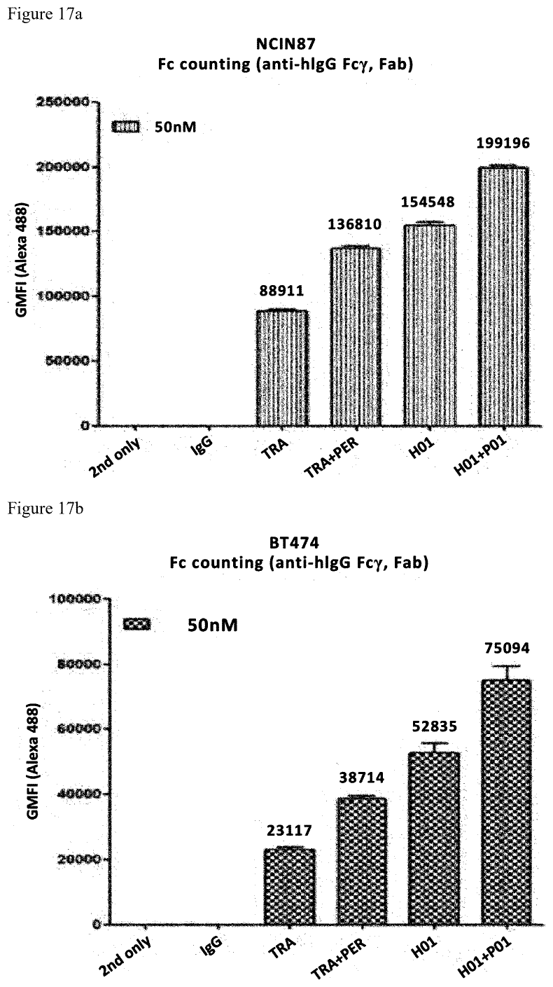

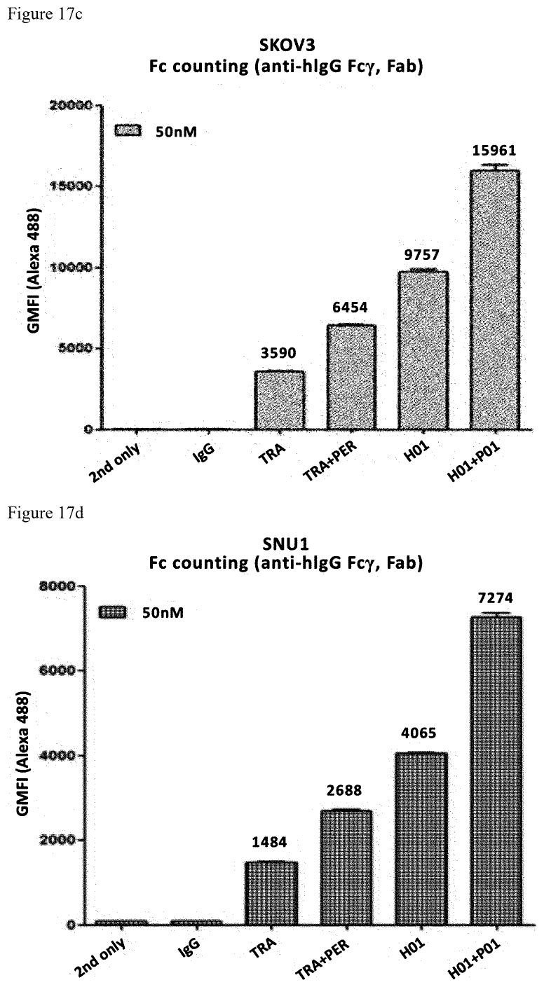

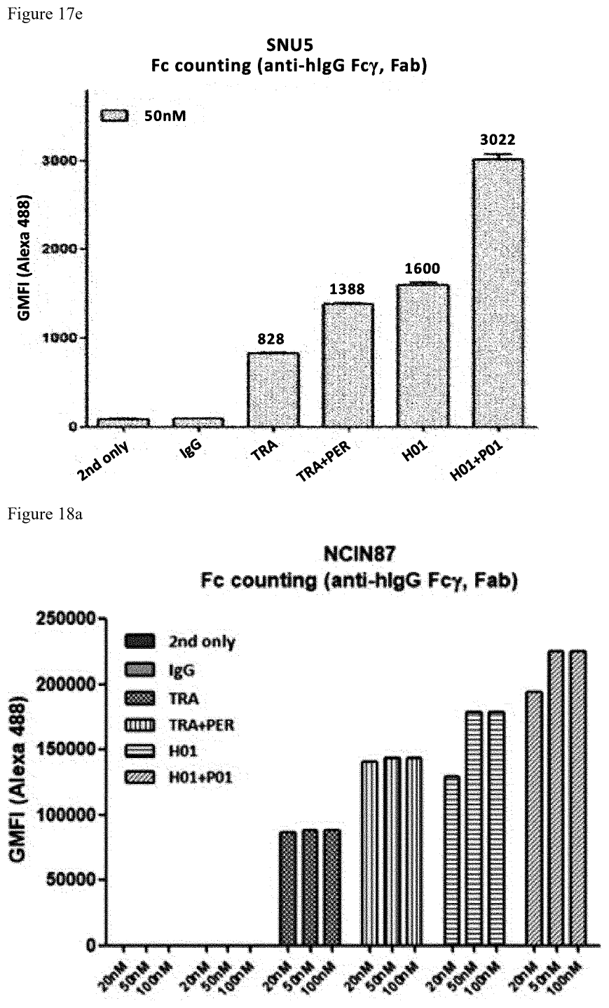

NCI-N87, BT474, SK-OV-3, SNU1, and SNU5 cancer cell lines used to quantify Fc loads of the antibodies on the surface of HER2-expressing cells were cultured in RPMI-1640+10% FBS medium. The cancer cell lines were treated with 50 nM human IgG1 (Bio X cell, BE0297), 50 nM trastuzumab (TRA), or 50 nM trastuzumab+50 nM pertuzumab (TRA+PER), 50 nM H01, 50 nM H01+50 nM P01 antibody at 4° C. for 30 minutes in a 96-well plate. Thereafter, they were treated with the Alexa 488 fluorescence-conjugated anti-human IgG Fcγ Fab antibody (Jackson ImmunoResearch, 109-547-008), and the antibody Fc bound to the cells was quantified using a flow cytometer (BD biosciences, FACSverse) (FIGS. 17a to 17e, Table 25). It was found that the fluorescence intensities of the 50 nM H01 alone group were higher than those of the 50 nM trastuzumab+50 nM pertuzumab (TRA+PER) combination group in the five cancer cell lines analyzed (FIGS. 17a to 17e, Table 25). Compared to treatment of 50 nM H01 alone, an additional increase in the Fc loads on the cancer cell surface was observed in the NCI-N87, BT474, SK-OV-3, SNU1, and SNU5 cancer cells when 50 nM P01 was treated in combination with 50 nM H01 (FIGS. 17a to 17e, Table 25).

TABLE 25

| | NCI- | | | | |
|---|---|---|---|---|---|
| GMFI | N87 | BT474 | SK-OV-3 | SNU-1 | SNU-5 |
| 50 nM Trastuzumab | 88911 | 23117 | 3590 | 1484 | 828 |
| 50 nM Trastuzumab + | 136810 | 38714 | 6454 | 2688 | 1388 |
| 50 nM Pertuzumab | | | | | |
| 50 nM H01 | 154548 | 52835 | 9757 | 4065 | 1600 |
| 50 nM H01 + 50 nM | 199196 | 75094 | 15961 | 7274 | 3022 |
| P01 | | | | | |

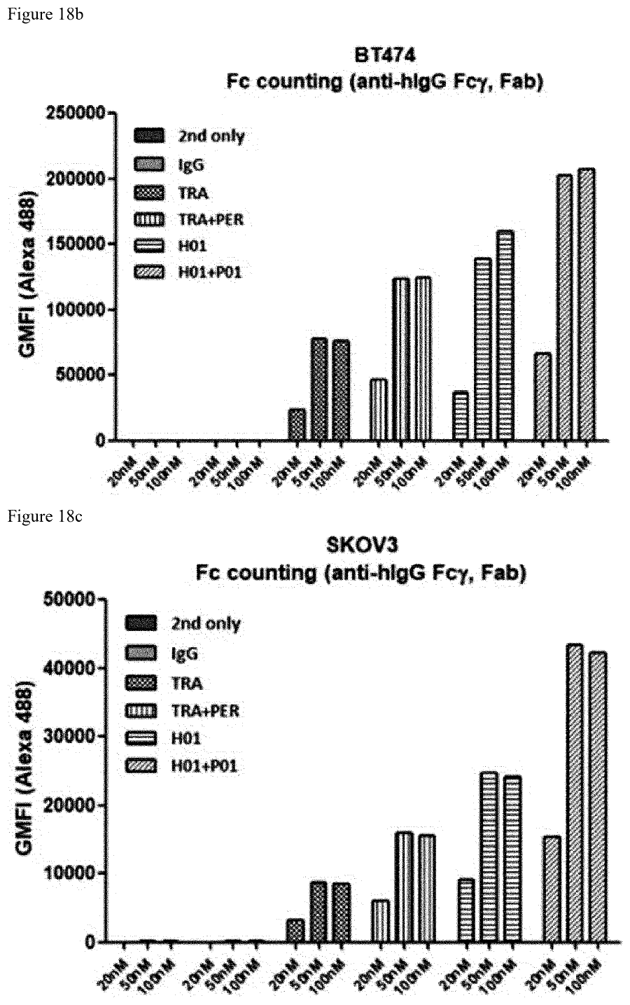

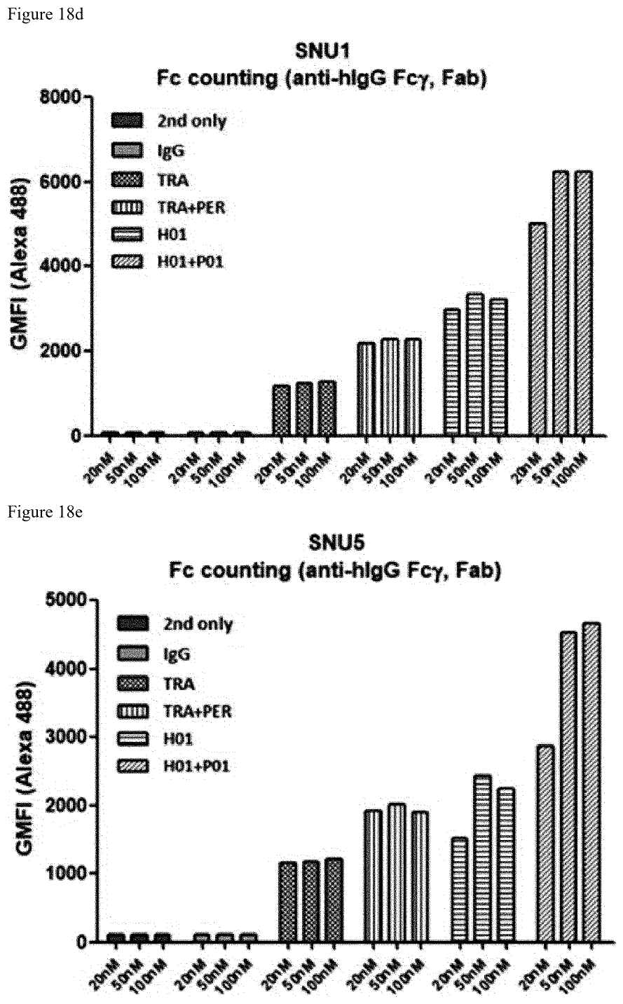

In order to determine the saturation concentration of antibodies binding to the cell surface, each test antibody was allowed to bind to a final concentration of 20, 50, and 100 nM, and the subsequent sampling process was carried out in the same manner as the above experimental conditions (FIGS. 18a to 18e). It was found that the saturation concentration of the antibody in the NCI-N87, BT474, SK-OV-3, SNU1, and SNU5 cancer cell lines was 50 nM (FIGS. 18a to 18e). It was found that treatment of 50 nM H01 alone results in more Fc loads on the surface of the five cell lines than treatment of 50 nM trastuzumab and 50 nM pertuzumab in combination (FIGS. 18a to 18e).

### Example 9. Analysis of Antibody Binding Affinity to HER2

The S239D and I332E mutations in the antibody Fc domain improve the affinity of the antibody for Fcγ receptors, which leads to improved effector function (Greg A. Lazar et al., PNAS, 2006). H01DE4 and P01DE4 were designed by introducing the S239D and I332E mutations in the H01 and P01 Fc domains (FIGS. 19a and 19b). H01DE4 was prepared by co-transfection of vectors capable of expressing the polypeptides corresponding to Fc-Hole-S239D-I332E (Table 26, SEQ ID NO: 42), TraH-G44C-Knob-S239D-I332E (Table 26, SEQ ID NO: 43), and TraL-Q100C-Knob-S239D-I332E (Table 26, SEQ ID NO: 44) into EXPICHO-S™ (Gibco, A29127). P01DE4 was prepared by co-transfection of vectors capable of expressing the polypeptides corresponding to Fc-Hole-S239D-I332E (Table 26, Table 27, and SEQ ID NO: 42), PerH-G44C-Knob-S239D-I332E (Table 26, Table 27, and SEQ ID NO: 45), and PerL-Q100C-Knob-S239D-I332E (Table 26, Table 27, and SEQ ID NO: 46) into EXPICHO-S™ (Gibco, A29127).

TABLE 26

| | | SEQ |
|---|---|---|
| Name | Sequence | ID NO |
| Fc-Hole- | DKTHTCPPCPAPELLGGPDVFLFPPKPKDTLMISRTPEVTCVVVDVSH | 42 |
| S239D-I332E | EDPEVKFNWYVDGVEVHNAKTKPREEQYNSTYRVVSVLTVLHQDW | |
| | LNGKEYKCKVSNKALPAPEEKTISKAKGQPREPQVCTLPPSREEMTK | |
| | NQVSLSCAVKGFYPSDIAVEWESNGQPENNYKTTPPVLDSDGSFFLV | |
| | SKLTVDKSRWQQGNVFSCSVMHEALHNHYTQKSLSLSPGK | |
| TraH-G44C- | EVQLVESGGGLVQPGGSLRLSCAASGFNIKDTYIHWVRQAPGKCLE | 43 |
| Knob-S239D- | WVARIYPTNGYTRYADSVKGRFTISADTSKNTAYLQMNSLRAEDTA | |
| I332E | VYYCSRWGGDGFYAMDYWGQGTLVTVSSASTKGPSVFPLAPSSKST | |
| | SGGTAALGCLVKDYFPEPVTVSWNSGALTSGVHTFPAVLQSSGLYSL | |
| | SSVVTVPSSSLGTQTYICNVNHKPSNTKVDKKVEPKSCDKTHTCPPCP | |
| | APELLGGPDVFLFPPKPKDTLMISRTPEVTCVVVDVSHEDPEVKFNW | |
| | YVDGVEVHNAKTKPREEQYNSTYRVVSVLTVLHQDWLNGKEYKCK | |
| | VSNKALPAPEEKTISKAKGQPREPQVYTLPPCREEMTKNQVSLWCLV | |
| | KGFYPSDIAVEWESNGQPENNYKTTPPVLDSDGSFFLYSKLTVDKSR | |
| | WQQGNVFSCSVMHEALHNHYTQKSLSLSPGK | |
| TraL-Q100C- | DIQMTQSPSSLSASVGDRVTITCRASQDVNTAVAWYQQKPGKAPKL | 44 |
| Knob-S239D- | LIYSASFLYSGVPSRFSGSRSGTDFTLTISSLQPEDFATYYCQQHYTTP | |
| I332E | PTFGCGTKVEIKRTVAAPSVFIFPPSDEQLKSGTASVVCLLNNFYPRE | |
| | AKVQWKVDNALQSGNSQESVTEQDSKDSTYSLSSTLTLSKADYEKH | |
| | KVYACEVTHQGLSSPVTKSFNRGECGGGGSGGGGSGGGGSEPKSSD | |
| | KTHTCPPCPAPELLGGPDVFLFPPKPKDTLMISRTPEVTCVVVDVSHE | |
| | DPEVKFNWYVDGVEVHNAKTKPREEQYNSTYRVVSVLTVLHQDWL | |
| | NGKEYKCKVSNKALPAPEEKTISKAKGQPREPQVYTLPPCREEMTKN | |
| | QVSLWCLVKGFYPSDIAVEWESNGQPENNYKTTPPVLDSDGSFFLYS | |
| | KLTVDKSRWQQGNVFSCSVMHEALHNHYTQKSLSLSPGK | |
| PerH-G44C- | EVQLVESGGGLVQPGGSLRLSCAASGFTFTDYTMDWVRQAPGKCLE | 45 |
| Knob-S239D- | WVADVNPNSGGSIYNQRFKGRFTLSVDRSKNTLYLQMNSLRAEDTA | |
| I332E | VYYCARNLGPSFYFDYWGQGTLVTVSSASTKGPSVFPLAPSSKSTSG | |
| | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PELLGGPDVFLFPPKPKDTLMISRTPEVTCVVVDVSHEDPEVKFNWY | |
| | VDGVEVHNAKTKPREEQYNSTYRVVSVLTVLHQDWLNGKEYKCKV | |
| | SNKALPAPEEKTISKAKGQPREPQVYTLPPCREEMTKNQVSLWCLVK | |
| | GFYPSDIAVEWESNGQPENNYKTTPPVLDSDGSFFLYSKLTVDKSRW | |
| | QQGNVFSCSVMHEALHNHYTQKSLSLSPGK | |
| PerL-Q100C- | DIQMTQSPSSLSASVGDRVTITCKASQDVSIGVAWYQQKPGKAPKLLI | 46 |
| Knob-S239D- | YSASYRYTGVPSRFSGSGSGTDFTLTISSLQPEDFATYYCQQYYIYPY | |
| I332E | TFGCGTKVEIKRTVAAPSVFIFPPSDEQLKSGTASVVCLLNNFYPREA | |
| | KVQWKVDNALQSGNSQESVTEQDSKDSTYSLSSTLTLSKADYEKHK | |
| | VYACEVTHQGLSSPVTKSFNRGECGGGGSGGGGSGGGGSEPKSSDK | |
| | THTCPPCPAPELLGGPDVFLFPPKPKDTLMISRTPEVTCVVVDVSHED | |
| | PEVKFNWYVDGVEVHNAKTKPREEQYNSTYRVVSVLTVLHQDWLN | |
| | GKEYKCKVSNKALPAPEEKTISKAKGQPREPQVYTLPPCREEMTKNQ | |
| | VSLWCLVKGFYPSDIAVEWESNGQPENNYKTTPPVLDSDGSFFLYSK | |
| | LTVDKSRWQQGNVFSCSVMHEALHNHYTQKSLSLSPGK | |

TABLE 27

| Name | Sequence | SEQ ID NO |
|---|---|---|
| Fc-Hole- | GACAAAACTCACACATGCCCACCGTGCCCAGCACCTGAACTCCTG | 479 |
| S239D-I332E | GGGGGACCGGATGTCTTCCTCTTCCCCCCAAAACCCAAGGACACC | 480 |
| | CTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCA | |
| | CCAGGACTGGCTGAATGGCAAGGAGTACAAGTGCAAGGTCTCCA | |
| | ACAAAGCCCTCCCAGCCCCCGAAGAGAAAACCATCTCCAAAGCC | |
| | AAAGGGCAGCCCCGAGAACCACAGGTGTGCACCCTGCCCCCATCC | |
| | CGGGAGGAGATGACCAAGAACCAGGTCAGCCTGAGCTGCGCGGT | |
| | CAAAGGCTTCTATCCCAGCGACATCGCCGTGGAGTGGGAGAGCAA | |
| | TGGGCAGCCGGAGAACAACTACAAGACCACGCCTCCCGTGCTGG | |
| | ACTCCGACGGCTCCTTCTTCCTCGTGAGCAAGCTCACCGTGGACA | |
| | AGAGCAGGTGGCAGCAGGGGAACGTCTTCTCATGCTCCGTGATGC | |
| | ATGAGGCTCTGCACAACCACTACACGCAGAAGAGCCTCTCCCTGT | |
| | CTCCGGGTAAA | |
| TraH-G44C- | GAAGTTCAACTTGTGGAGTCTGGAGGCGGTTTGGTACAACCTGGA | |
| Knob-S239D- | GGTTCACTTCGTTTGTCATGTGCTGCCTCTGGGTTTAATATCAAGG | |
| I332E | ATACTTATATTCATTGGGTTCGCCAGGCACCCGGAAAATGCCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACCAAAGGACCTAGTGTTTTTCCTCTTGCCCCTTCCTCAAAGT | |
| | CTACCTCTGGGGGGACAGCCGCTCTGGGCTGCCTGGTCAAGGATT | |
| | ATTTCCCAGAGCCTGTCACTGTCAGTTGGAACTCTGGAGCCTTGAC | |
| | TTCTGGTGTTCATACATTTCCTGCTGTCCTTCAAAGCAGCGGCTTG | |
| | TACTCATTGTCTTCTGTTGTGACAGTACCCTCAAGCAGCCTCGGCA | |
| | CTCAGACATACATCTGCAATGTCAACCACAAACCCTCAAATACAA | |
| | AGGTAGATAAAAAAGTCGAACCAAAGTCTTGTGACAAAACTCAC | |
| | ACGTGCCCACCGTGCCCAGCACCTGAACTCCTGGGGGGACCGGAT | |
| | GTCTTCCTCTTCCCCCCAAAACCCAAGGACACCCTCATGATCTCCC | |
| | GGACCCCTGAGGTCACATGCGTGGTGGTGGACGTGAGCCACGAA | |
| | GACCCTGAGGTCAAGTTCAACTGGTACGTGGACGGCGTGGAGGTG | |
| | CATAATGCCAAGACAAAGCCGCGGGAGGAGCAGTACAACAGCAC | |
| | GTACCGTGTGGTCAGCGTCCTCACCGTCCTGCACCAGGACTGGCT | |
| | GAATGGCAAGGAGTACAAGTGCAAGGTCTCCAACAAAGCCCTCC | |
| | CAGCCCCCGAAGAGAAAACCATCTCCAAAGCCAAAGGGCAGCCC | |
| | CGAGAACCACAGGTGTACACCCTGCCCCCATGCCGGGAGGAGAT | |
| | GACCAAGAACCAGGTCAGCCTGTGGTGCCTGGTCAAAGGCTTCTA | |
| | TCCCAGCGACATCGCCGTGGAGTGGGAGAGCAATGGGCAGCCGG | |
| | AGAACAACTACAAGACCACGCCTCCCGTGCTGGACTCCGACGGCT | |
| | CCTTCTTCCTCTACAGCAAGCTCACCGTGGACAAGAGCAGGTGGC | |
| | AGCAGGGGAACGTCTTCTCATGCTCCGTGATGCATGAGGCTCTGC | |
| | ACAACCACTACACGCAGAAGAGCCTCTCCCTGTCTCCGGGTAAA | |
| TraL-Q100C- | GACATTCAGATGACCCAATCCCCCTCTAGCCTTTCAGCCTCCGTAG | 481 |
| Knob-S239D- | GGGACAGAGTTACAATAACCTGTAGGGCTAGTCAGGACGTTAATA | |
| I332E | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGATGTGGCACAAAAGTAGAGATTA | |
| | AGCGTACGGTGGCAGCTCCCAGCGTTTTTATCTTTCCCCCATCCGA | |
| | CGAGCAGCTCAAGAGTGGCACTGCCTCTGTAGTTTGTTTGCTGAAT | |
| | AACTTCTATCCACGTGAAGCAAAAGTACAGTGGAAGGTCGATAAT | |
| | GCCCTTCAGAGCGGTAACAGCCAAGAAAGTGTTACCGAGCAAGA | |
| | TTCCAAAGATTCCACTTACAGTCTGTCCAGCACATTGACACTGAGT | |
| | AAGGCTGATTACGAAAAACACAAGGTGTACGCATGCGAGGTGAC | |
| | ACACCAAGGTCTTTCATCTCCTGTAACTAAGAGCTTTAACCGGGG | |
| | AGAATGTGGTGGTGGGGGCAGCGGGGGCGGAGGTAGTGGAGGCG | |
| | GCGGTAGTGAACCAAAGAGTAGTGACAAAACTCACACGTGCCCA | |
| | CCGTGCCCAGCACCTGAACTCCTGGGGGGACCGGATGTCTTCCTC | |
| | TTCCCCCCAAAACCCAAGGACACCCTCATGATCTCCCGGACCCCT | |
| | GAGGTCACATGCGTGGTGGTGGACGTGAGCCACGAAGACCCTGA | |
| | GGTCAAGTTCAACTGGTACGTGGACGGCGTGGAGGTGCATAATGC | |
| | CAAGACAAAGCCGCGGGAGGAGCAGTACAACAGCACGTACCGTG | |
| | TGGTCAGCGTCCTCACCGTCCTGCACCAGGACTGGCTGAATGGCA | |
| | AGGAGTACAAGTGCAAGGTCTCCAACAAAGCCCTCCCAGCCCCCG | |
| | AAGAGAAAACCATCTCCAAAGCCAAAGGGCAGCCCCGAGAACCA | |
| | CAGGTGTACACCCTGCCCCCATGCCGGGAGGAGATGACCAAGAA | |
| | CCAGGTCAGCCTGTGGTGCCTGGTCAAAGGCTTCTATCCCAGCGA | |
| | CATCGCCGTGGAGTGGGAGAGCAATGGGCAGCCGGAGAACAACT | |
| | ACAAGACCACGCCTCCCGTGCTGGACTCCGACGGCTCCTTCTTCCT | |
| | CTACAGCAAGCTCACCGTGGACAAGAGCAGGTGGCAGCAGGGGA | |
| | ACGTCTTCTCATGCTCCGTGATGCATGAGGCTCTGCACAACCACTA | |
| | CACGCAGAAGAGCCTCTCCCTGTCTCCGGGTAAA | |
| PerH-G44C- | GAAGTACAGTTGGTGGAGTCTGGTGGAGGTCTTGTACAACCTGGC | 482 |
| Knob-S239D- | GGGAGTTTGCGGCTTTCCTGCGCTGCAAGCGGGTTTACCTTCACCG | |
| I332E | ATTATACCATGGATTGGGTACGCCAAGCCCCTGGTAAGTGCCTTG | |
| | AGTGGGTTGCCGATGTAAACCCTAATTCCGGAGGAAGTATCTATA | |
| | ATCAACGCTTCAAGGGCCGATTCACTCTGAGTGTGGATCGAAGCA | |
| | AGAACACCTTGTACTTGCAGATGAATTCCTTGCGGGCTGAAGACA | |
| | CAGCCGTCTATTACTGCGCCCGAAATTTGGGGCCTTCATTCTATTT | |
| | TGACTATTGGGGTCAGGGAACTCTGGTAACTGTTTCAAGTGCTAG | |
| | CACCAAAGGACCTAGTGTTTTTCCTCTTGCCCCTTCCTCAAAGTCT | |
| | ACCTCTGGGGGGACAGCCGCTCTGGGCTGCCTGGTCAAGGATTAT | |
| | TTCCCAGAGCCTGTCACTGTCAGTTGGAACTCTGGAGCCTTGACTT | |
| | CTGGTGTTCATACATTTCCTGCTGTCCTTCAAAGCAGCGGCTTGTA | |
| | CTCATTGTCTTCTGTTGTGACAGTACCCTCAAGCAGCCTCGGCACT | |
| | CAGACATACATCTGCAATGTCAACCACAAACCCTCAAATACAAAG | |
| | GTAGATAAAAAAGTCGAACCAAAGTCTTGTGACAAAACTCACAC | |
| | GTGCCCACCGTGCCCAGCACCTGAACTCCTGGGGGGACCGGATGT | |
| | CTTCCTCTTCCCCCCAAAACCCAAGGACACCCTCATGATCTCCCGG | |
| | ACCCCTGAGGTCACATGCGTGGTGGTGGACGTGAGCCACGAAGAC | |
| | CCTGAGGTCAAGTTCAACTGGTACGTGGACGGCGTGGAGGTGCAT | |
| | AATGCCAAGACAAAGCCGCGGGAGGAGCAGTACAACAGCACGTA | |
| | CCGTGTGGTCAGCGTCCTCACCGTCCTGCACCAGGACTGGCTGAA | |
| | TGGCAAGGAGTACAAGTGCAAGGTCTCCAACAAAGCCCTCCCAGC | |
| | CCCCGAAGAGAAAACCATCTCCAAAGCCAAAGGGCAGCCCCGAG | |
| | AACCACAGGTGTACACCCTGCCCCCATGCCGGGAGGAGATGACCA | |
| | AGAACCAGGTCAGCCTGTGGTGCCTGGTCAAAGGCTTCTATCCCA | |
| | GCGACATCGCCGTGGAGTGGGAGAGCAATGGGCAGCCGGAGAAC | |
| | AACTACAAGACCACGCCTCCCGTGCTGGACTCCGACGGCTCCTTC | |
| | TTCCTCTACAGCAAGCTCACCGTGGACAAGAGCAGGTGGCAGCAG | |
| | GGGAACGTCTTCTCATGCTCCGTGATGCATGAGGCTCTGCACAAC | |
| | CACTACACGCAGAAGAGCCTCTCCCTGTCTCCGGGTAAA | |
| PerL-Q100C- | GATATTCAAATGACCCAAAGTCCAAGTTCCCTTTCAGCATCTGTCG | 483 |
| Knob-S239D- | GTGATCGAGTCACTATAACTTGTAAGGCCAGCCAAGACGTTAGCA | |
| I332E | TAGGAGTAGCATGGTACCAACAAAAGCCTGGGAAGGCTCCCAAA | |
| | CTTCTCATTTATTCTGCTTCCTACCGATATACTGGTGTCCCAAGTA | |
| | GATTTTCTGGCAGCGGATCTGGAACTGATTTTACATTGACTATCAG | |
| | CTCCCTCCAGCCTGAGGACTTCGCTACTTATTACTGCCAACAGTAC | |
| | TACATTTATCCCTATACATTCGGTTGTGGGACCAAAGTAGAGATC | |
| | AAACGTACGGTGGCAGCTCCCAGCGTTTTTATCTTTCCCCCATCCG | |
| | ACGAGCAGCTCAAGAGTGGCACTGCCTCTGTAGTTTGTTTGCTGA | |
| | ATAACTTCTATCCACGTGAAGCAAAAGTACAGTGGAAGGTCGATA | |
| | ATGCCCTTCAGAGCGGTAACAGCCAAGAAAGTGTTACCGAGCAA | |
| | GATTCCAAAGATTCCACTTACAGTCTGTCCAGCACATTGACACTG | |
| | AGTAAGGCTGATTACGAAAAACACAAGGTGTACGCATGCGAGGT | |
| | GACACACCAAGGTCTTTCATCTCCTGTAACTAAGAGCTTTAACCG | |
| | GGGAGAATGTGGTGGTGGGGGCAGCGGGGGCGGAGGTAGTGGAG | |
| | GCGGCGGTAGTGAACCAAAGAGTAGTGACAAAACTCACACGTGC | |
| | CCACCGTGCCCAGCACCTGAACTCCTGGGGGGACCGGATGTCTTC | |
| | CTCTTCCCCCCAAAACCCAAGGACACCCTCATGATCTCCCGGACC | |
| | CCTGAGGTCACATGCGTGGTGGTGGACGTGAGCCACGAAGACCCT | |
| | GAGGTCAAGTTCAACTGGTACGTGGACGGCGTGGAGGTGCATAAT | |
| | GCCAAGACAAAGCCGCGGGAGGAGCAGTACAACAGCACGTACCG | |
| | TGTGGTCAGCGTCCTCACCGTCCTGCACCAGGACTGGCTGAATGG | |
| | CAAGGAGTACAAGTGCAAGGTCTCCAACAAAGCCCTCCCAGCCCC | |
| | CGAAGAGAAAACCATCTCCAAAGCCAAAGGGCAGCCCCGAGAAC | |
| | CACAGGTGTACACCCTGCCCCCATGCCGGGAGGAGATGACCAAG | |
| | AACCAGGTCAGCCTGTGGTGCCTGGTCAAAGGCTTCTATCCCAGC | |
| | GACATCGCCGTGGAGTGGGAGAGCAATGGGCAGCCGGAGAACAA | |
| | CTACAAGACCACGCCTCCCGTGCTGGACTCCGACGGCTCCTTCTTC | |
| | CTCTACAGCAAGCTCACCGTGGACAAGAGCAGGTGGCAGCAGGG | |
| | GAACGTCTTCTCATGCTCCGTGATGCATGAGGCTCTGCACAACCA | |
| | CTACACGCAGAAGAGCCTCTCCCTGTCTCCGGGTAAA | |

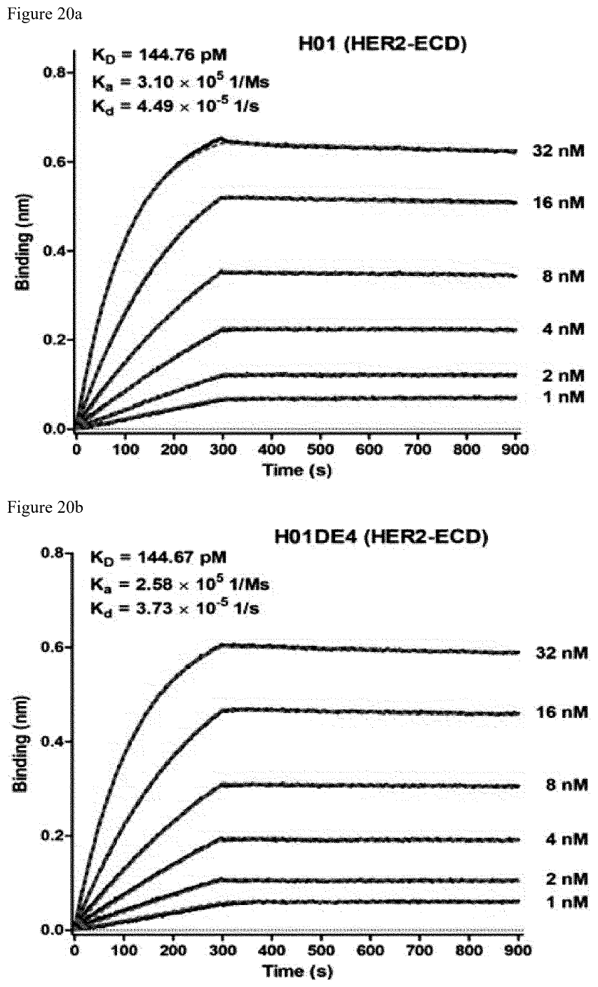

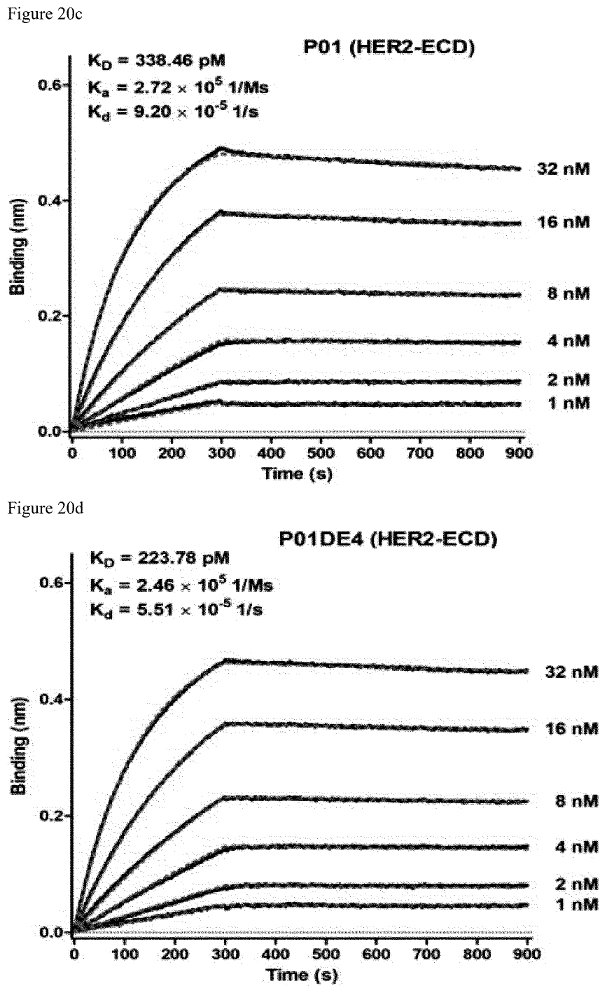

Binding characteristics were analyzed at 25° C. using Octet Red96e (Sartorius), a bio-layer interferometry (BLI). The buffer used for analysis was 10× Kinetics Buffer (Sartorius, 18-1042) diluted in PBS pH 7.4 (Gibco, 10010), and analysis plate was agitated at 1,000 rpm. The human HER2 recombinant protein (R&D systems, 1129-ER) was loaded onto the Anti-Penta-HIS (HIS1K) biosensor (Sartorius, 18-5120). To measure the binding rate constant (Kₐ), 1 to 32 nM of H01, H01DE4, P01, and P01DE4 were allowed to bind to loaded antigen for 300 seconds and then the dissociation rate constant (K_{d}) was determined after 600 seconds of dissociation in Kinetics Buffer. Kₐ and K_{d} values were measured through a 1:1 binding model in Octet analysis software (Sartorius), and the equilibrium dissociation constant (K_{D}) value was determined (FIGS. 20a to 20d, Table 28).

TABLE 28

| | K_{D}(pM) | Kₐ(1/Ms) | K_{d}(1/s) |
|---|---|---|---|
| H01 | 144.76 | 3.10 × 10⁵ | 4.49 × 10⁻⁵ |
| H01DE4 | 144.67 | 2.58 × 10⁵ | 3.73 × 10⁻⁵ |
| P01 | 338.46 | 2.72 × 10⁵ | 9.20 × 10⁻⁵ |
| P01DE4 | 223.78 | 2.46 × 10⁵ | 5.51 × 10⁻⁵ |

### Example 10. Analysis of Antibody Binding Affinity to Fcγ Receptors

The binding constants of each antibody to Fcγ receptors at 25° C. were analyzed using Octet Red96e (Sartorius). Anti-Penta-HIS (HIS1K) biosensor (Sartorius, 18-5120) was used, and human FcγRI (R&D systems, 1257-FC) or human FcγIIA (R&D systems, 1330-CD) or human FcγRIIIA (176V isoform, R&D systems, 4325-FC) containing a His tag were loaded onto the biosensor.

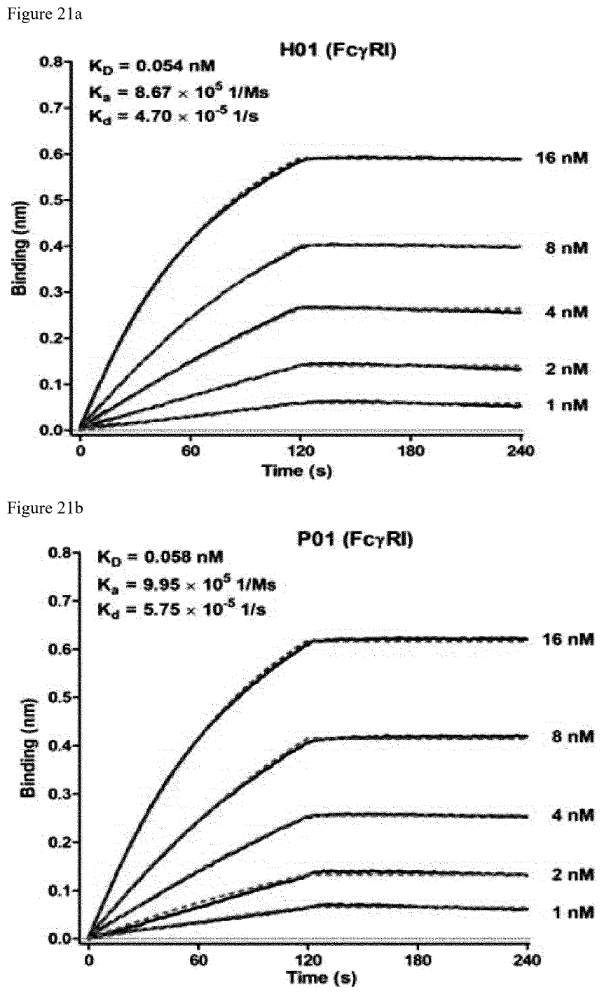

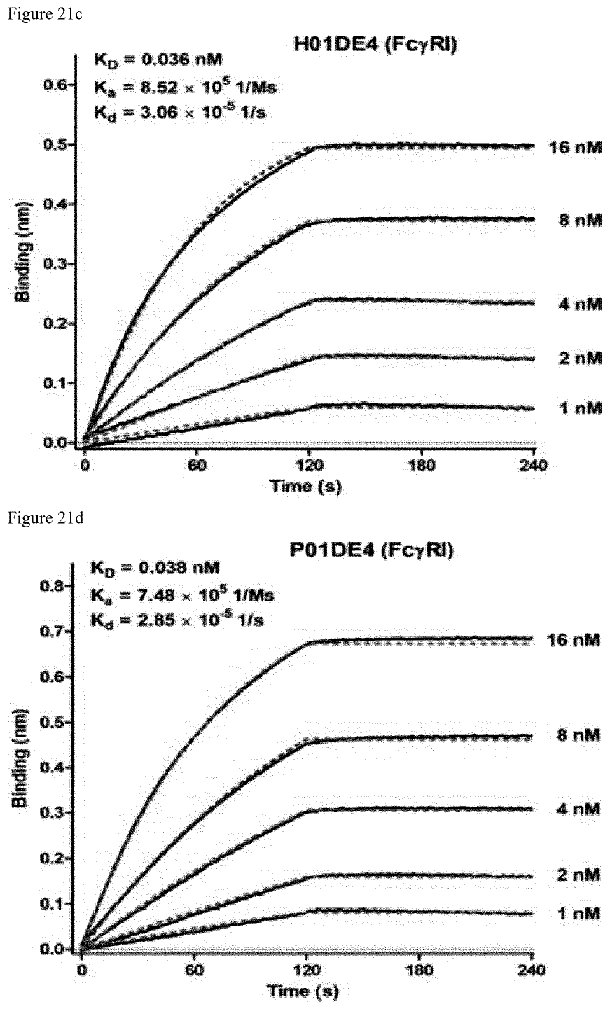

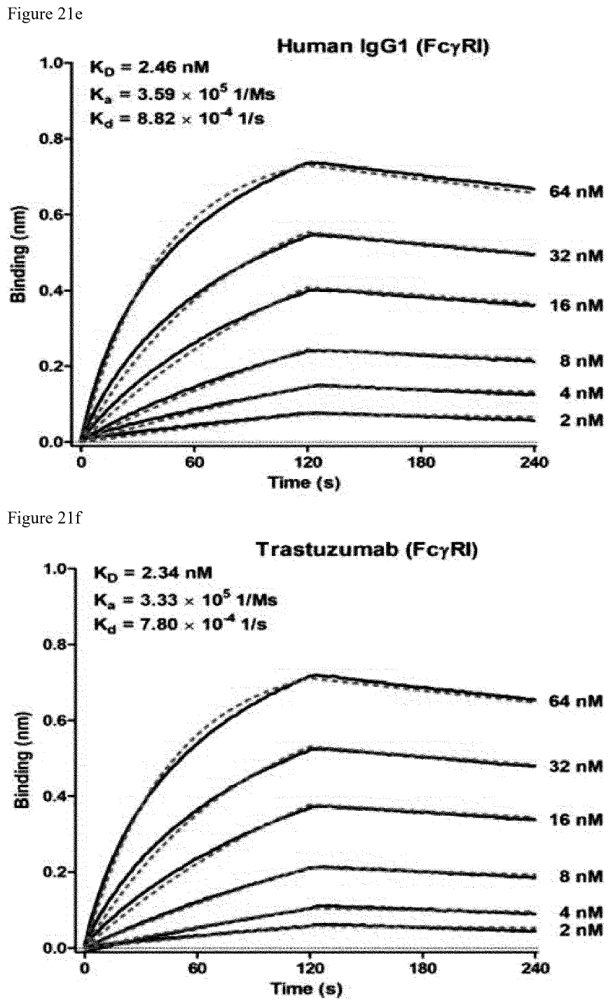

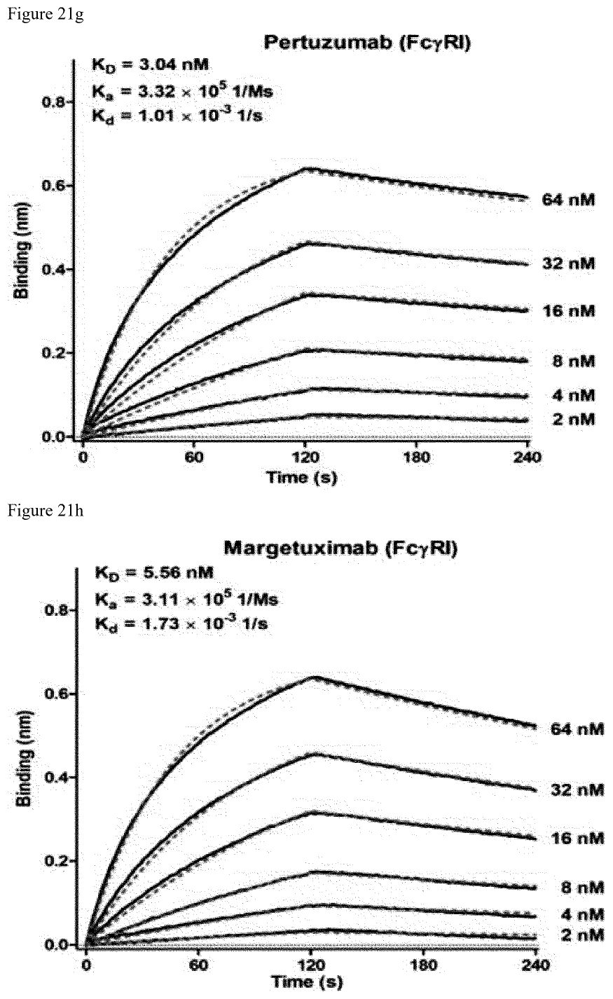

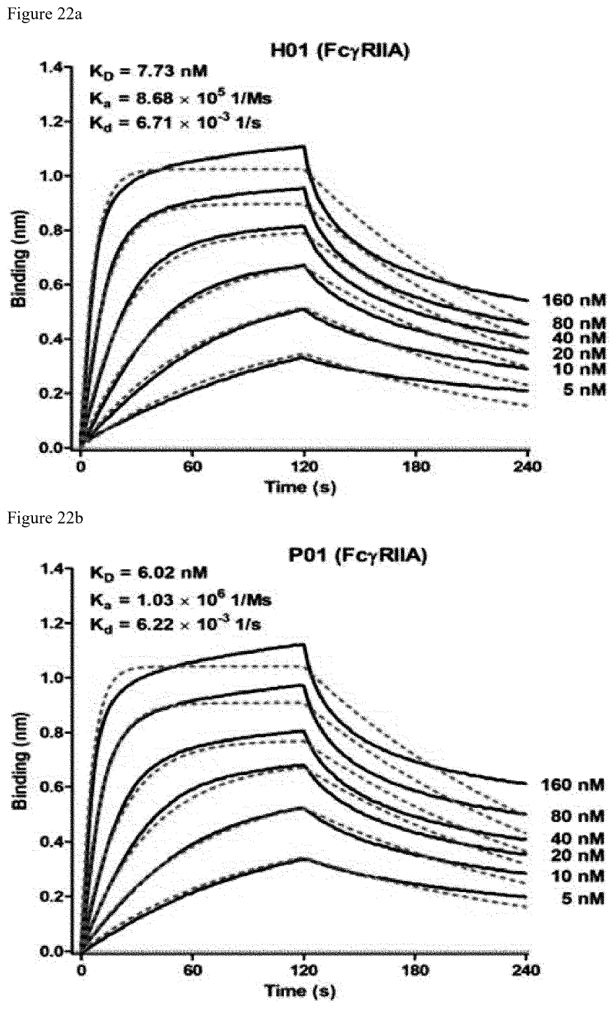

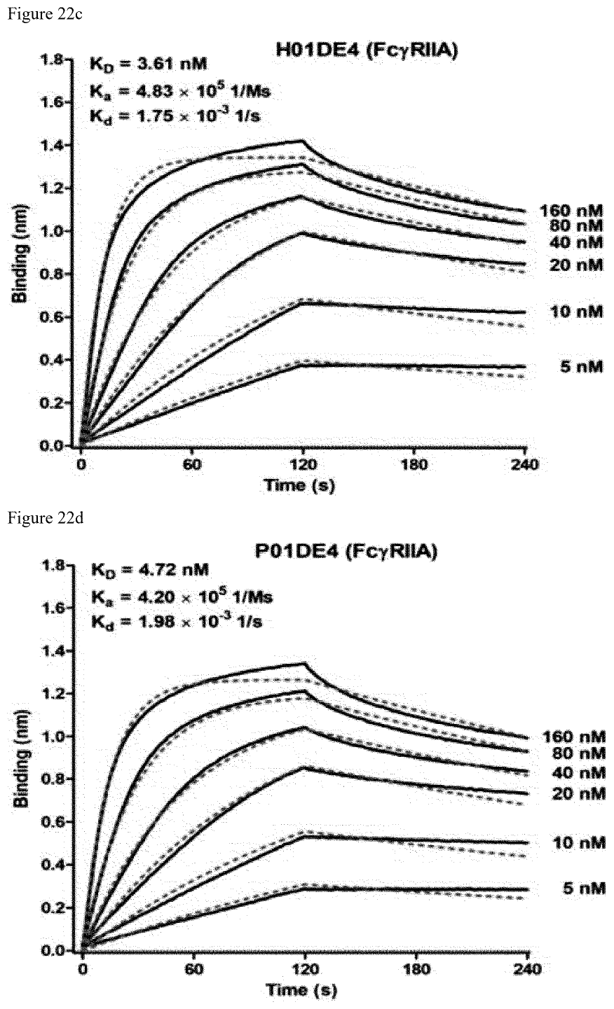

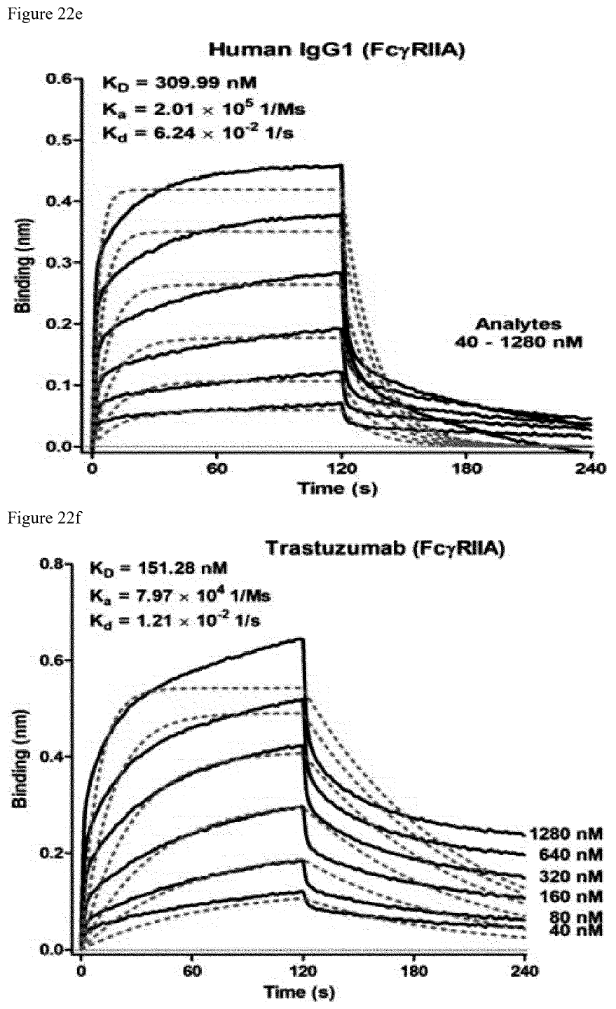

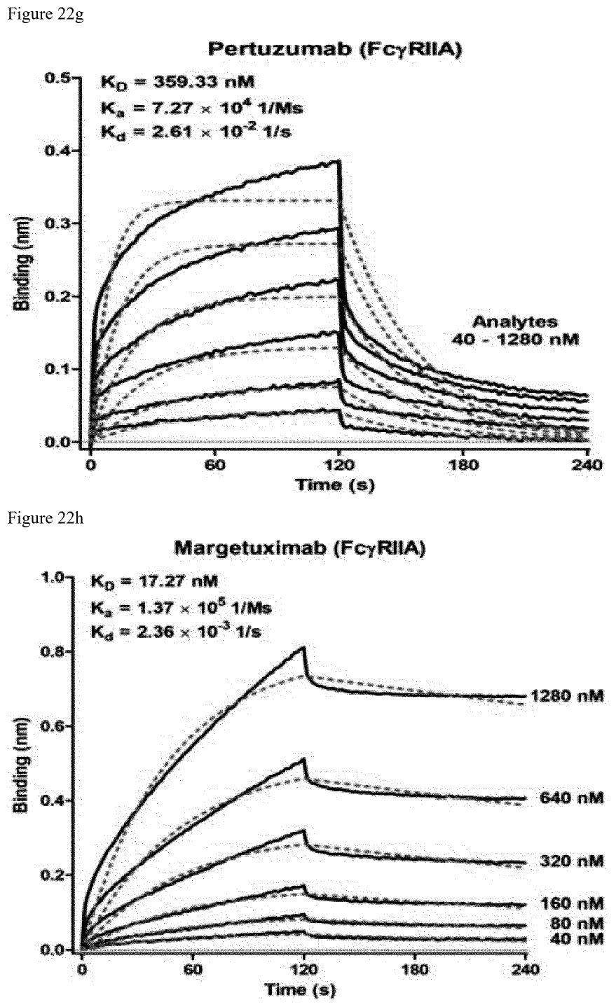

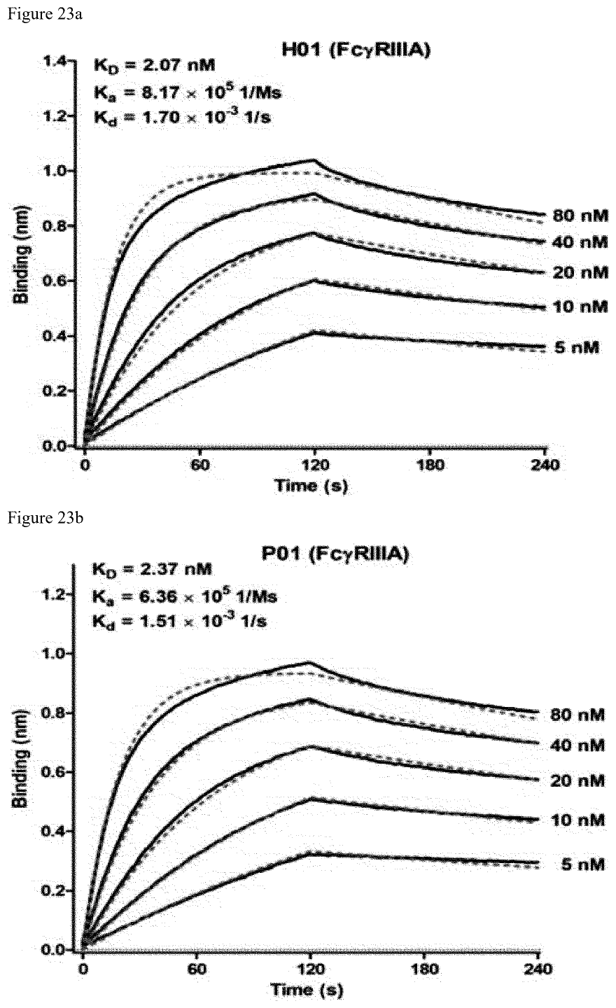

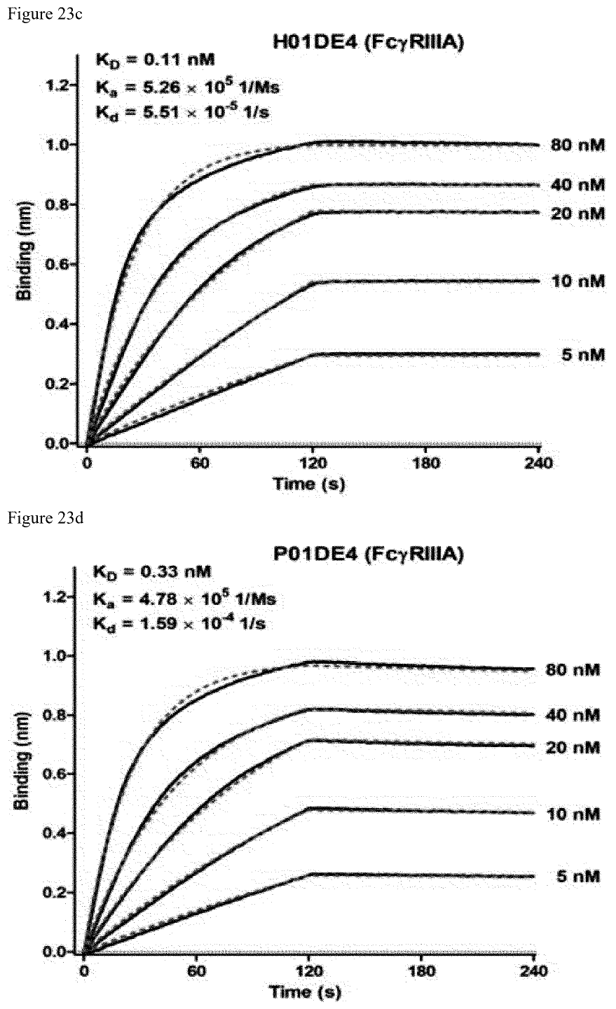

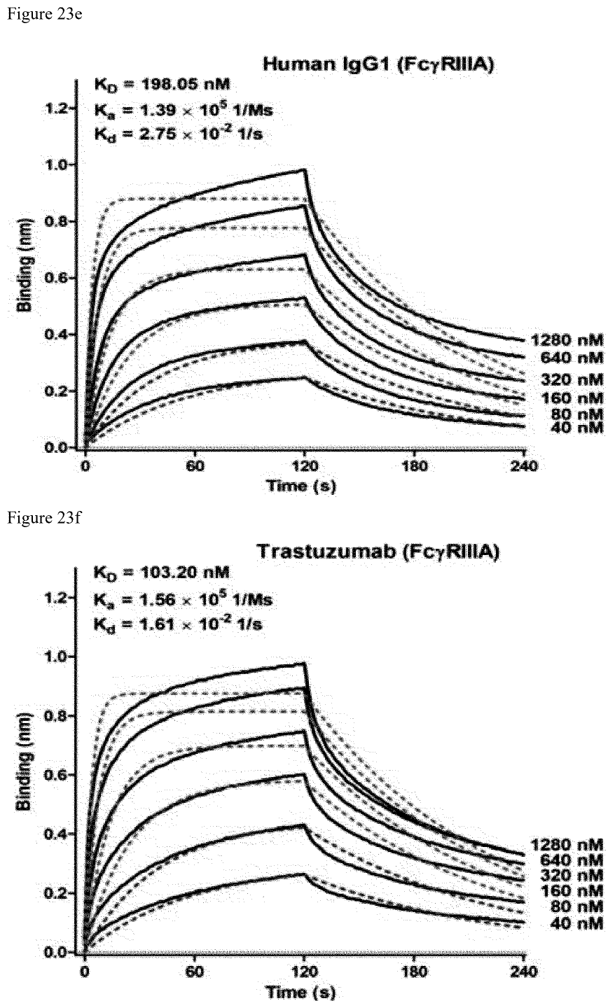

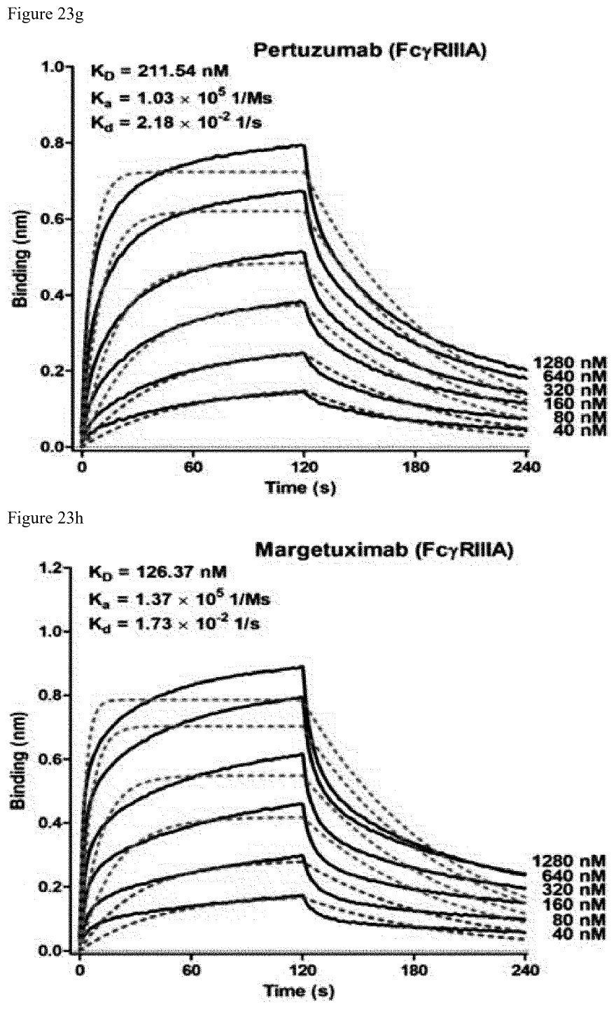

The association rate constants (Kₐ) and the dissociation rate constants (K_{d}) of H01, P01, H01DE4, P01DE4, human IgG1 (Bio X cell, BE0297), trastuzumab, pertuzumab, and margetuximab to the biosensor loaded with an antigen was determined. Kₐ and K_{d} values were calculated through a 1:1 binding model in Octet analysis software (Sartorius), and the equilibrium dissociation constant (K_{D}) value was determined (FIGS. 21a to 21h, FIGS. 22a to 22h, FIGS. 23a to 23h, Table 29).

TABLE 29

| | FcγRI(CD64) | | | FcγRIIA(CD32A, 131R) | | | FcγRIIIA(CD16A, 176V) | | |
|---|---|---|---|---|---|---|---|---|---|
| Antigen | KD | Ka | Kd | KD | Ka | Kd | KD | Ka | Kd |
| Antibody | (nM) | (1/Ms) | (1/s) | (nM) | (1/Ms) | (1/s) | (nM) | (1/Ms) | (1/s) |
| H01 | 0.054 | 8.67E+05 | 4.70E−05 | 7.73 | 8.68E+05 | 6.71E−03 | 2.08 | 8.17E+05 | 1.70E−03 |
| P01 | 0.058 | 9.95E+05 | 5.75E−05 | 6.04 | 1.03E+06 | 6.22E−03 | 2.37 | 6.36E+05 | 1.51E−03 |
| H01DE4 | 0.036 | 8.52E+05 | 3.06E−05 | 3.62 | 4.83E+05 | 1.75E−03 | 0.10 | 5.26E+05 | 5.51E−05 |
| P01DE4 | 0.038 | 7.48E+05 | 2.85E−05 | 4.71 | 4.20E+05 | 1.98E−03 | 0.33 | 4.78E+05 | 1.59E−04 |
| Human IgG1 | 2.457 | 3.59E+05 | 8.82E−04 | 310.45 | 2.01E+05 | 6.24E−02 | 197.84 | 1.39E+05 | 2.75E−02 |
| Trastuzumab | 2.342 | 3.33E+05 | 7.80E−04 | 151.82 | 7.97E+04 | 1.21E−02 | 103.21 | 1.56E+05 | 1.61E−02 |
| Pertuzumab | 3.042 | 3.32E+05 | 1.01E−03 | 359.01 | 7.27E+04 | 2.61E−02 | 211.65 | 1.03E+05 | 2.18E−02 |
| Margetuximab | 5.563 | 3.11E+05 | 1.73E−03 | 17.23 | 1.37E+05 | 2.36E−03 | 126.28 | 1.37E+05 | 1.73E−02 |

### Example 11. Pharmacokinetic (PK) Analysis in Rats

H01, P01 trastuzumab, and pertuzumab were administered at 10 mg/kg to 7-week-old male Sprague-Dawley rats (ORIENT BIO INC.) via the intravenous (i.v.) route. Blood samples were collected in 5 minutes, 15 minutes, 30 minutes, 1 hour, 2 hours, 4 hours, 8 hours, 1 day, 2 days, 3 days, 7 days, 10 days, 14 days, 21 days, 28 days, 35 days, and 42 days after administration. Thereafter, only the serum was separated from the blood for analysis. The antibody concentration in serum was measured by ELISA.

Briefly, a 96-well ELISA plate (Corning, 3590) was coated with human HER2 recombinant protein (R&D systems, 1129-ER) and stored overnight at 4° C., and the sera obtained at each time were appropriately diluted, and allowed to bind to the coated human HER2. Peroxidase-conjugated anti-human Fab goat antibody (Invitrogen, 31482) was used to detect H01, P01 trastuzumab, and pertuzumab.

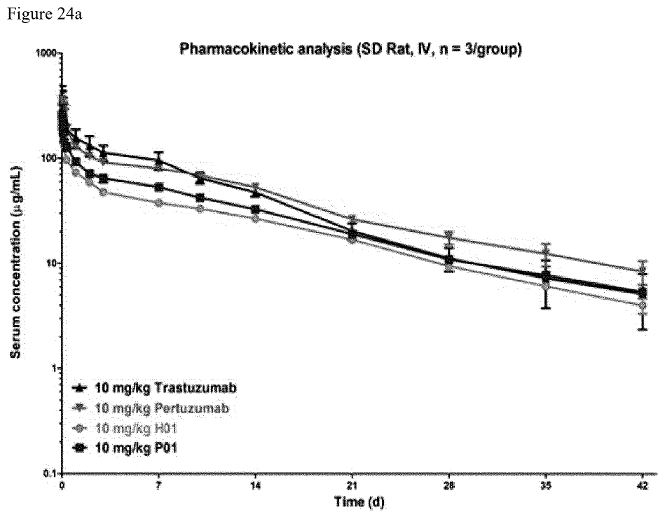

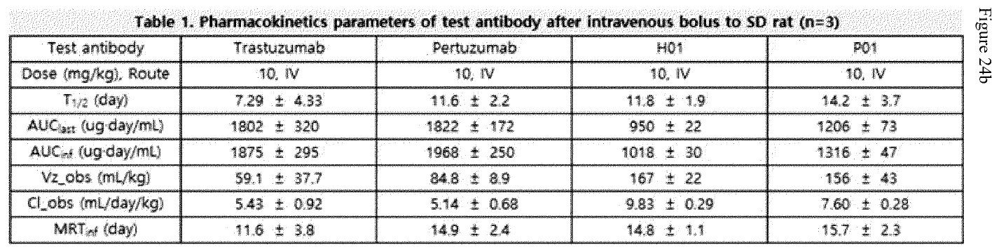

Standard samples of H01, P01, trastuzumab, and pertuzumab were prepared, and the concentrations of the analytes at each time point were quantified based on a standard curve created from naive rat serum containing different concentrations of the antibody standards. The half-lives of intravenously administered H01, P01, trastuzumab, and pertuzumab were determined to be approximately 11.8 days, 14.2 days, 7.3 days, and 11.6 days, respectively (FIGS. 24a and 24b, Table 30). The engineered novel antibodies, H01 and P01, were found to have similar PK parameters to the parental humanized antibodies trastuzumab and pertuzumab.

TABLE 30

| | Trastuzumab | Pertuzumab | H01 | P01 |
|---|---|---|---|---|
| Dose (mg/kg), | 10, IV | 10, IV | 10, IV | 10, IV |
| Route | | | | |
| T_{1/2} (day) | 7.3 ± 4.3 | 11.6 ± 2.2 | 11.8 ± 1.9 | 14.2 ± 3.7 |
| AUCₗₐₛₜ | 1802 ± 320 | 1822 ± 172 | 950 ± 22 | 1206 ± 73 |
| (μg*day/mL) | | | | |
| AUC_{inf} | 1875 ± 295 | 1968 ± 250 | 1018 ± 30 | 1316 ± 47 |
| (μg*day/mL) | | | | |
| Vz_obs | 59.1 ± 37.7 | 84.8 ± 8.9 | 167 ± 22 | 156 ± 43 |
| (mL/kg) | | | | |
| Cl_obs | 5.4 ± 0.9 | 5.1 ± 0.7 | 9.8 ± 0.3 | 7.6 ± 0.3 |
| (mL/day/kg) | | | | |
| MRT_{inf} (day) | 11.6 ± 3.8 | 14.9 ± 2.4 | 14.8 ± 1.1 | 15.7 ± 2.3 |

### Example 12. Design, Preparation and Analysis of Novel Antibody that Recognizes Two Epitopes of HER2

In order to construct an antibody that recognizes two epitopes of HER2 protein, a biparatopic antibody HP51 was designed by connecting the V domains of trastuzumab and pertuzumab via a linker (FIG. 25). In order to minimize the decrease in binding affinity due to interference between different V domains, linkers with variable lengths connecting the V domains were tested. At the same time, as an attempt to improve the physical integrity of the antibody, 16 variants were designed in which Cys substitution mutations capable of forming a disulfide bond in the V domain were introduced (FIG. 26, Table 31). According to Kabat numbering system, mutations were introduced only at position 44 for the heavy chain and position 100 for the light chain (Table 31). In Table 31, the VH linker with 6 amino acid residues was designated as VH-S-Linker, the VH linker with 13 amino acid residues was designated as VH-L-Linker, the VL linker with 6 amino acid residues was designated as VL-S-Linker, and the VL linker with 13 amino acid residues was designated as VL-L-Linker.

TABLE 31

| | VH domain | | | VH domain | | |
|---|---|---|---|---|---|---|
| Clone | VH1 44 | Linker | VH2 44 | VL1 100 | Linker | VL2 100 |
| name | (Upper) | length | (Lower) | (Upper) | length | (Lower) |
| HP501 | Gly (G) | 6 | Gly (G) | Gln (Q) | 6 | Gln (Q) |
| HP502 | Cys (C) | 6 | Gly (G) | Cys (C) | 6 | Gln (Q) |
| HP503 | Gly (G) | 6 | Cys (C) | Gln (Q) | 6 | Cys (C) |
| HP504 | Cys (C) | 6 | Cys (C) | Cys (C) | 6 | Cys (C) |
| HP505 | Gly (G) | 6 | Gly (G) | Gln (Q) | 13 | Gln (Q) |
| HP506 | Cys (C) | 6 | Gly (G) | Cys (C) | 13 | Gln (Q) |
| HP507 | Gly (G) | 6 | Cys (C) | Gln (Q) | 13 | Cys (C) |
| HP508 | Cys (C) | 6 | Cys (C) | Cys (C) | 13 | Cys (C) |
| HP509 | Gly (G) | 13 | Gly (G) | Gln (Q) | 6 | Gln (Q) |
| HP510 | Cys (C) | 13 | Gly (G) | Cys (C) | 6 | Gln (Q) |
| HP511 | Gly (G) | 13 | Cys (C) | Gln (Q) | 6 | Cys (C) |
| HP512 | Cys (C) | 13 | Cys (C) | Cys (C) | 6 | Cys (C) |
| HP513 | Gly (G) | 13 | Gly (G) | Gln (Q) | 13 | Gln (Q) |
| HP514 | Cys (C) | 13 | Gly (G) | Cys (C) | 13 | Gln (Q) |
| HP515 | Gly (G) | 13 | Cys (C) | Gln (Q) | 13 | Cys (C) |
| HP516 | Cys (C) | 13 | Cys (C) | Cys (C) | 13 | Cys (C) |

For HP51, expression vectors consisting of the sequences corresponding to Fc-Hole (SEQ ID NO: 7), TN-S-PH-Knob (SEQ ID NO: 51), and TL-S-PL-Knob (SEQ ID NO: 59) were co-transfected into EXPICHO-S™ (Gibco, A29127) (Tables 31, 32, 33, and 34), and purification and analysis were performed in the same manner as described in Example 1. Expression, purification and analysis were performed for HP502 to HP516 in the same manner as mentioned above, and the composition of the expression vector is shown in detail

TABLE 32

| | | SEQ |
|---|---|---|
| Name | Sequence | ID NO |
| VH-S-Linker | ASTKGP | 47 |
| VH-L-Linker | ASTKGPSVFPLAP | 48 |
| VL-S-Linker | RTVAAP | 49 |
| VL-L-Linker | RTVAAPSVFIFPP | 50 |
| TH-S- | EVQLVESGGGLVQPGGSLRLSCAASGFNIKDTYIHWVRQAPGKGLE | 51 |
| PH-Knob | WVARIYPTNGYTRYADSVKGRFTISADTSKNTAYLQMNSLRAEDTA | |
| | VYYCSRWGGDGFYAMDYWGQGTLVTVSSASTKGPEVQLVESGGGL | |
| | VQPGGSLRLSCAASGFTFTDYTMDWVRQAPGKGLEWVADVNPNSG | |
| | GSIYNQRFKGRFTLSVDRSKNTLYLQMNSLRAEDTAVYYCARNLGPS | |
| | FYFDYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQ | |
| | TYICNVNHKPSNTKVDKKVEPKSCDKTHTCPPCPAPELLGGPSVFLFP | |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMH | |
| | EALHNHYTQKSLSLSPGK | |
| THC-S- | EVQLVESGGGLVQPGGSLRLSCAASGFNIKDTYIHWVRQAPGKCLE | 52 |
| PH-Knob | WVARIYPTNGYTRYADSVKGRFTISADTSKNTAYLQMNSLRAEDTA | |
| | VYYCSRWGGDGFYAMDYWGQGTLVTVSSASTKGPEVQLVESGGGL | |
| | VQPGGSLRLSCAASGFTFTDYTMDWVRQAPGKGLEWVADVNPNSG | |
| | GSIYNQRFKGRFTLSVDRSKNTLYLQMNSLRAEDTAVYYCARNLGPS | |
| | FYFDYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQ | |
| | TYICNVNHKPSNTKVDKKVEPKSCDKTHTCPPCPAPELLGGPSVFLFP | |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMH | |
| | EALHNHYTQKSLSLSPGK | |
| TH-S- | EVQLVESGGGLVQPGGSLRLSCAASGFNIKDTYIHWVRQAPGKGLE | 53 |
| PHC-Knob | WVARIYPTNGYTRYADSVKGRFTISADTSKNTAYLQMNSLRAEDTA | |
| | VYYCSRWGGDGFYAMDYWGQGTLVTVSSASTKGPEVQLVESGGGL | |
| | VQPGGSLRLSCAASGFTFTDYTMDWVRQAPGKCLEWVADVNPNSG | |
| | GSIYNQRFKGRFTLSVDRSKNTLYLQMNSLRAEDTAVYYCARNLGPS | |
| | FYFDYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQ | |
| | TYICNVNHKPSNTKVDKKVEPKSCDKTHTCPPCPAPELLGGPSVFLFP | |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMH | |
| | EALHNHYTQKSLSLSPGK | |
| THC-S- | EVQLVESGGGLVQPGGSLRLSCAASGFNIKDTYIHWVRQAPGKCLE | 54 |
| PHC-Knob | WVARIYPTNGYTRYADSVKGRFTISADTSKNTAYLQMNSLRAEDTA | |
| | VYYCSRWGGDGFYAMDYWGQGTLVTVSSASTKGPEVQLVESGGGL | |
| | VQPGGSLRLSCAASGFTFTDYTMDWVRQAPGKCLEWVADVNPNSG | |
| | GSIYNQRFKGRFTLSVDRSKNTLYLQMNSLRAEDTAVYYCARNLGPS | |
| | FYFDYWGQGTLVTVSSASTKGPSVFPLAPSSKSTSGGTAALGCLVKD | |
| | YFPEPVTVSWNSGALTSGVHTFPAVLQSSGLYSLSSVVTVPSSSLGTQ | |
| | TYICNVNHKPSNTKVDKKVEPKSCDKTHTCPPCPAPELLGGPSVFLFP | |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMH | |
| | EALHNHYTQKSLSLSPGK | |
| TH-L- | EVQLVESGGGLVQPGGSLRLSCAASGFNIKDTYIHWVRQAPGKGLE | 55 |
| PH-Knob | WVARIYPTNGYTRYADSVKGRFTISADTSKNTAYLQMNSLRAEDTA | |
| | VYYCSRWGGDGFYAMDYWGQGTLVTVSSASTKGPSVFPLAPEVQL | |
| | VESGGGLVQPGGSLRLSCAASGFTFTDYTMDWVRQAPGKGLEWVA | |
| | DVNPNSGGSIYNQRFKGRFTLSVDRSKNTLYLQMNSLRAEDTAVYY | |
| | CARNLGPSFYFDYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| THC-L- | EVQLVESGGGLVQPGGSLRLSCAASGFNIKDTYIHWVRQAPGKCLE | 56 |
| PH-Knob | WVARIYPTNGYTRYADSVKGRFTISADTSKNTAYLQMNSLRAEDTA | |
| | VYYCSRWGGDGFYAMDYWGQGTLVTVSSASTKGPSVFPLAPEVQL | |
| | VESGGGLVQPGGSLRLSCAASGFTFTDYTMDWVRQAPGKGLEWVA | |
| | DVNPNSGGSIYNQRFKGRFTLSVDRSKNTLYLQMNSLRAEDTAVYY | |
| | CARNLGPSFYFDYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| TH-L- | EVQLVESGGGLVQPGGSLRLSCAASGFNIKDTYIHWVRQAPGKGLE | 57 |
| PHC-Knob | WVARIYPTNGYTRYADSVKGRFTISADTSKNTAYLQMNSLRAEDTA | |
| | VYYCSRWGGDGFYAMDYWGQGTLVTVSSASTKGPSVFPLAPEVQL | |
| | VESGGGLVQPGGSLRLSCAASGFTFTDYTMDWVRQAPGKCLEWVA | |
| | DVNPNSGGSIYNQRFKGRFTLSVDRSKNTLYLQMNSLRAEDTAVYY | |
| | CARNLGPSFYFDYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| THC-L- | EVQLVESGGGLVQPGGSLRLSCAASGFNIKDTYIHWVRQAPGKCLE | 58 |
| PHC-Knob | WVARIYPTNGYTRYADSVKGRFTISADTSKNTAYLQMNSLRAEDTA | |
| | VYYCSRWGGDGFYAMDYWGQGTLVTVSSASTKGPSVFPLAPEVQL | |
| | VESGGGLVQPGGSLRLSCAASGFTFTDYTMDWVRQAPGKCLEWVA | |
| | DVNPNSGGSIYNQRFKGRFTLSVDRSKNTLYLQMNSLRAEDTAVYY | |
| | CARNLGPSFYFDYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| TL-S- | DIQMTQSPSSLSASVGDRVTITCRASQDVNTAVAWYQQKPGKAPKL | 59 |
| PL-Knob | LIYSASFLYSGVPSRFSGSRSGTDFTLTISSLQPEDFATYYCQQHYTTP | |
| | PTFGQGTKVEIKRTVAAPDIQMTQSPSSLSASVGDRVTITCKASQDVS | |
| | IGVAWYQQKPGKAPKLLIYSASYRYTGVPSRFSGSGSGTDFTLTISSL | |
| | QPEDFATYYCQQYYIYPYTFGQGTKVEIKRTVAAPSVFIFPPSDEQLK | |
| | SGTASVVCLLNNFYPREAKVQWKVDNALQSGNSQESVTEQDSKDST | |
| | YSLSSTLTLSKADYEKHKVYACEVTHQGLSSPVTKSFNRGECGGGGS | |
| | GGGGSGGGGSEPKSSDKTHTCPPCPAPELLGGPSVFLFPPKPKDTLMI | |
| | SRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREP | |
| | QVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQ | |
| | KSLSLSPGK | |
| TLC-S- | DIQMTQSPSSLSASVGDRVTITCRASQDVNTAVAWYQQKPGKAPKL | 60 |
| PL-Knob | LIYSASFLYSGVPSRFSGSRSGTDFTLTISSLQPEDFATYYCQQHYTTP | |
| | PTFGCGTKVEIKRTVAAPDIQMTQSPSSLSASVGDRVTITCKASQDVSI | |
| | GVAWYQQKPGKAPKLLIYSASYRYTGVPSRFSGSGSGTDFTLTISSLQ | |
| | PEDFATYYCQQYYIYPYTFGQGTKVEIKRTVAAPSVFIFPPSDEQLKS | |
| | GTASVVCLLNNFYPREAKVQWKVDNALQSGNSQESVTEQDSKDSTY | |
| | SLSSTLTLSKADYEKHKVYACEVTHQGLSSPVTKSFNRGECGGGGSG | |
| | GGGSGGGGSEPKSSDKTHTCPPCPAPELLGGPSVFLFPPKPKDTLMIS | |
| | RTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREP | |
| | QVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQ | |
| | KSLSLSPGK | |
| TL-S- | DIQMTQSPSSLSASVGDRVTITCRASQDVNTAVAWYQQKPGKAPKL | 61 |
| PLC-Knob | LIYSASFLYSGVPSRFSGSRSGTDFTLTISSLQPEDFATYYCQQHYTTP | |
| | PTFGQGTKVEIKRTVAAPDIQMTQSPSSLSASVGDRVTITCKASQDVS | |
| | IGVAWYQQKPGKAPKLLIYSASYRYTGVPSRFSGSGSGTDFTLTISSL | |
| | QPEDFATYYCQQYYIYPYTFGCGTKVEIKRTVAAPSVFIFPPSDEQLK | |
| | SGTASVVCLLNNFYPREAKVQWKVDNALQSGNSQESVTEQDSKDST | |
| | YSLSSTLTLSKADYEKHKVYACEVTHQGLSSPVTKSFNRGECGGGGS | |
| | GGGGSGGGGSEPKSSDKTHTCPPCPAPELLGGPSVFLFPPKPKDTLMI | |
| | SRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREP | |
| | QVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQ | |
| | KSLSLSPGK | |
| TLC-S- | DIQMTQSPSSLSASVGDRVTITCRASQDVNTAVAWYQQKPGKAPKL | 62 |
| PLC-Knob | LIYSASFLYSGVPSRFSGSRSGTDFTLTISSLQPEDFATYYCQQHYTTP | |
| | PTFGCGTKVEIKRTVAAPDIQMTQSPSSLSASVGDRVTITCKASQDVSI | |
| | GVAWYQQKPGKAPKLLIYSASYRYTGVPSRFSGSGSGTDFTLTISSLQ | |
| | PEDFATYYCQQYYIYPYTFGCGTKVEIKRTVAAPSVFIFPPSDEQLKS | |
| | GTASVVCLLNNFYPREAKVQWKVDNALQSGNSQESVTEQDSKDSTY | |
| | SLSSTLTLSKADYEKHKVYACEVTHQGLSSPVTKSFNRGECGGGGSG | |
| | GGGSGGGGSEPKSSDKTHTCPPCPAPELLGGPSVFLFPPKPKDTLMIS | |
| | RTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREP | |
| | QVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQ | |
| | KSLSLSPGK | |
| TL-L- | DIQMTQSPSSLSASVGDRVTITCRASQDVNTAVAWYQQKPGKAPKL | 63 |
| PL-Knob | LIYSASFLYSGVPSRFSGSRSGTDFTLTISSLQPEDFATYYCQQHYTTP | |
| | PTFGQGTKVEIKRTVAAPSVFIFPPDIQMTQSPSSLSASVGDRVTITCK | |
| | ASQDVSIGVAWYQQKPGKAPKLLIYSASYRYTGVPSRFSGSGSGTDF | |
| | TLTISSLQPEDFATYYCQQYYIYPYTFGQGTKVEIKRTVAAPSVFIFPP | |
| | SDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGE | |
| | CGGGGSGGGGSGGGGSEPKSSDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| TLC-L- | DIQMTQSPSSLSASVGDRVTITCRASQDVNTAVAWYQQKPGKAPKL | 64 |
| PL-Knob | LIYSASFLYSGVPSRFSGSRSGTDFTLTISSLQPEDFATYYCQQHYTTP | |
| | PTFGCGTKVEIKRTVAAPSVFIFPPDIQMTQSPSSLSASVGDRVTITCK | |
| | ASQDVSIGVAWYQQKPGKAPKLLIYSASYRYTGVPSRFSGSGSGTDF | |
| | TLTISSLQPEDFATYYCQQYYIYPYTFGQGTKVEIKRTVAAPSVFIFPP | |
| | SDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGE | |
| | CGGGGSGGGGSGGGGSEPKSSDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| TL-L- | DIQMTQSPSSLSASVGDRVTITCRASQDVNTAVAWYQQKPGKAPKL | 65 |
| PLC-Knob | LIYSASFLYSGVPSRFSGSRSGTDFTLTISSLQPEDFATYYCQQHYTTP | |
| | PTFGCGTKVEIKRTVAAPSVFIFPPDIQMTQSPSSLSASVGDRVTITCK | |
| | ASQDVSIGVAWYQQKPGKAPKLLIYSASYRYTGVPSRFSGSGSGTDF | |
| | TLTISSLQPEDFATYYCQQYYIYPYTFGQGTKVEIKRTVAAPSVFIFPP | |
| | SDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGE | |
| | CGGGGSGGGGSGGGGSEPKSSDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| TLC-L- | DIQMTQSPSSLSASVGDRVTITCRASQDVNTAVAWYQQKPGKAPKL | 66 |
| PLC-Knob | LIYSASFLYSGVPSRFSGSRSGTDFTLTISSLQPEDFATYYCQQHYTTP | |
| | PTFGCGTKVEIKRTVAAPSVFIFPPDIQMTQSPSSLSASVGDRVTITCK | |
| | ASQDVSIGVAWYQQKPGKAPKLLIYSASYRYTGVPSRFSGSGSGTDF | |
| | TLTISSLQPEDFATYYCQQYYIYPYTFGCGTKVEIKRTVAAPSVFIFPP | |
| | SDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGE | |
| | CGGGGSGGGGSGGGGSEPKSSDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |

TABLE 33

| | | SEQ |
|---|---|---|
| Name | Sequence | ID NO |
| VH-S-Linker | GCTAGCACAAAAGGACCT | 484 |
| VH-L-Linker | GCTAGCACAAAAGGACCTAGTGTTTTCCCACTGGCTCCA | 485 |
| VL-S-Linker | CGTACGGTGGCTGCTCCA | 486 |
| VL-L-Linker | CGTACGGTGGCTGCTCCATCCGTTTTTATCTTTCCCCCA | 487 |
| TH-S-PH-Knob | GAAGTTCAACTTGTGGAGTCTGGAGGCGGTTTGGTACAACCTGGA | 488 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTGAAGTACAGTTGGTGGAGTCTGGTGGAGG | |
| | TCTTGTACAACCTGGCGGGAGTTTGCGGCTTTCCTGCGCTGCAAGC | |
| | GGGTTTACCTTCACCGATTATACCATGGATTGGGTACGCCAAGCC | |
| | CCTGGTAAGGGCCTTGAGTGGGTTGCCGATGTAAACCCTAATTCC | |
| | GGAGGAAGTATCTATAATCAACGCTTCAAGGGCCGATTCACTCTG | |
| | AGTGTGGATCGAAGCAAGAACACCTTGTACTTGCAGATGAATTCC | |
| | TTGCGGGCTGAAGACACAGCCGTCTATTACTGCGCCCGAAATTTG | |
| | GGGCCTTCATTCTATTTTGACTATTGGGGTCAGGGAACTCTGGTAA | |
| | CTGTTTCAAGTGCTAGCACCAAAGGACCTAGTGTTTTTCCTCTTGC | |
| | CCCTTCCTCAAAGTCTACCTCTGGGGGGACAGCCGCTCTGGGCTG | |
| | CCTGGTCAAGGATTATTTCCCAGAGCCTGTCACTGTCAGTTGGAAC | |
| | TCTGGAGCCTTGACTTCTGGTGTTCATACATTTCCTGCTGTCCTTC | |
| | AAAGCAGCGGCTTGTACTCATTGTCTTCTGTTGTGACAGTACCCTC | |
| | AAGCAGCCTCGGCACTCAGACATACATCTGCAATGTCAACCACAA | |
| | ACCCTCAAATACAAAGGTAGATAAAAAAGTCGAACCAAAGTCTT | |
| | GTGACAAAACTCACACGTGCCCACCGTGCCCAGCACCTGAACTCC | |
| | TGGGGGGACCGTCAGTCTTCCTCTTCCCCCCAAAACCCAAGGACA | |
| | CCCTCATGATCTCCCGGACCCCTGAGGTCACATGCGTGGTGGTGG | |
| | ACGTGAGCCACGAAGACCCTGAGGTCAAGTTCAACTGGTACGTGG | |
| | ACGGCGTGGAGGTGCATAATGCCAAGACAAAGCCGCGGGAGGAG | |
| | CAGTACAACAGCACGTACCGTGTGGTCAGCGTCCTCACCGTCCTG | |
| | CACCAGGACTGGCTGAATGGCAAGGAGTACAAGTGCAAGGTCTC | |
| | CAACAAAGCCCTCCCAGCCCCCATCGAGAAAACCATCTCCAAAGC | |
| | CAAAGGGCAGCCCCGAGAACCACAGGTGTACACCCTGCCCCCATG | |
| | CCGGGAGGAGATGACCAAGAACCAGGTCAGCCTGTGGTGCCTGG | |
| | TCAAAGGCTTCTATCCCAGCGACATCGCCGTGGAGTGGGAGAGCA | |
| | ATGGGCAGCCGGAGAACAACTACAAGACCACGCCTCCCGTGCTG | |
| | GACTCCGACGGCTCCTTCTTCCTCTACAGCAAGCTCACCGTGGAC | |
| | AAGAGCAGGTGGCAGCAGGGGAACGTCTTCTCATGCTCCGTGATG | |
| | CATGAGGCTCTGCACAACCACTACACGCAGAAGAGCCTCTCCCTG | |
| | TCTCCGGGTAAA | |
| THC-S-PH-Knob | GAAGTTCAACTTGTGGAGTCTGGAGGCGGTTTGGTACAACCTGGA | 489 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTGAAGTACAGTTGGTGGAGTCTGGTGGAGG | |
| | TCTTGTACAACCTGGCGGGAGTTTGCGGCTTTCCTGCGCTGCAAGC | |
| | GGGTTTACCTTCACCGATTATACCATGGATTGGGTACGCCAAGCC | |
| | CCTGGTAAGGGCCTTGAGTGGGTTGCCGATGTAAACCCTAATTCC | |
| | GGAGGAAGTATCTATAATCAACGCTTCAAGGGCCGATTCACTCTG | |
| | AGTGTGGATCGAAGCAAGAACACCTTGTACTTGCAGATGAATTCC | |
| | TTGCGGGCTGAAGACACAGCCGTCTATTACTGCGCCCGAAATTTG | |
| | GGGCCTTCATTCTATTTTGACTATTGGGGTCAGGGAACTCTGGTAA | |
| | CTGTTTCAAGTGCTAGCACCAAAGGACCTAGTGTTTTTCCTCTTGC | |
| | CCCTTCCTCAAAGTCTACCTCTGGGGGGACAGCCGCTCTGGGCTG | |
| | CCTGGTCAAGGATTATTTCCCAGAGCCTGTCACTGTCAGTTGGAAC | |
| | TCTGGAGCCTTGACTTCTGGTGTTCATACATTTCCTGCTGTCCTTC | |
| | AAAGCAGCGGCTTGTACTCATTGTCTTCTGTTGTGACAGTACCCTC | |
| | AAGCAGCCTCGGCACTCAGACATACATCTGCAATGTCAACCACAA | |
| | ACCCTCAAATACAAAGGTAGATAAAAAAGTCGAACCAAAGTCTT | |
| | GTGACAAAACTCACACGTGCCCACCGTGCCCAGCACCTGAACTCC | |
| | TGGGGGGACCGTCAGTCTTCCTCTTCCCCCCAAAACCCAAGGACA | |
| | CCCTCATGATCTCCCGGACCCCTGAGGTCACATGCGTGGTGGTGG | |
| | ACGTGAGCCACGAAGACCCTGAGGTCAAGTTCAACTGGTACGTGG | |
| | ACGGCGTGGAGGTGCATAATGCCAAGACAAAGCCGCGGGAGGAG | |
| | CAGTACAACAGCACGTACCGTGTGGTCAGCGTCCTCACCGTCCTG | |
| | CACCAGGACTGGCTGAATGGCAAGGAGTACAAGTGCAAGGTCTC | |
| | CAACAAAGCCCTCCCAGCCCCCATCGAGAAAACCATCTCCAAAGC | |
| | CAAAGGGCAGCCCCGAGAACCACAGGTGTACACCCTGCCCCCATG | |
| | CCGGGAGGAGATGACCAAGAACCAGGTCAGCCTGTGGTGCCTGG | |
| | TCAAAGGCTTCTATCCCAGCGACATCGCCGTGGAGTGGGAGAGCA | |
| | ATGGGCAGCCGGAGAACAACTACAAGACCACGCCTCCCGTGCTG | |
| | GACTCCGACGGCTCCTTCTTCCTCTACAGCAAGCTCACCGTGGAC | |
| | AAGAGCAGGTGGCAGCAGGGGAACGTCTTCTCATGCTCCGTGATG | |
| | CATGAGGCTCTGCACAACCACTACACGCAGAAGAGCCTCTCCCTG | |
| | TCTCCGGGTAAA | |
| TH-S-PHC-Knob | GAAGTTCAACTTGTGGAGTCTGGAGGCGGTTTGGTACAACCTGGA | 490 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTGAAGTACAGTTGGTGGAGTCTGGTGGAGG | |
| | TCTTGTACAACCTGGCGGGAGTTTGCGGCTTTCCTGCGCTGCAAGC | |
| | GGGTTTACCTTCACCGATTATACCATGGATTGGGTACGCCAAGCC | |
| | CCTGGTAAGTGCCTTGAGTGGGTTGCCGATGTAAACCCTAATTCC | |
| | GGAGGAAGTATCTATAATCAACGCTTCAAGGGCCGATTCACTCTG | |
| | AGTGTGGATCGAAGCAAGAACACCTTGTACTTGCAGATGAATTCC | |
| | TTGCGGGCTGAAGACACAGCCGTCTATTACTGCGCCCGAAATTTG | |
| | GGGCCTTCATTCTATTTTGACTATTGGGGTCAGGGAACTCTGGTAA | |
| | CTGTTTCAAGTGCTAGCACCAAAGGACCTAGTGTTTTTCCTCTTGC | |
| | CCCTTCCTCAAAGTCTACCTCTGGGGGGACAGCCGCTCTGGGCTG | |
| | CCTGGTCAAGGATTATTTCCCAGAGCCTGTCACTGTCAGTTGGAAC | |
| | TCTGGAGCCTTGACTTCTGGTGTTCATACATTTCCTGCTGTCCTTC | |
| | AAAGCAGCGGCTTGTACTCATTGTCTTCTGTTGTGACAGTACCCTC | |
| | AAGCAGCCTCGGCACTCAGACATACATCTGCAATGTCAACCACAA | |
| | ACCCTCAAATACAAAGGTAGATAAAAAAGTCGAACCAAAGTCTT | |
| | GTGACAAAACTCACACGTGCCCACCGTGCCCAGCACCTGAACTCC | |
| | TGGGGGGACCGTCAGTCTTCCTCTTCCCCCCAAAACCCAAGGACA | |
| | CCCTCATGATCTCCCGGACCCCTGAGGTCACATGCGTGGTGGTGG | |
| | ACGTGAGCCACGAAGACCCTGAGGTCAAGTTCAACTGGTACGTGG | |
| | ACGGCGTGGAGGTGCATAATGCCAAGACAAAGCCGCGGGAGGAG | |
| | CAGTACAACAGCACGTACCGTGTGGTCAGCGTCCTCACCGTCCTG | |
| | CACCAGGACTGGCTGAATGGCAAGGAGTACAAGTGCAAGGTCTC | |
| | CAACAAAGCCCTCCCAGCCCCCATCGAGAAAACCATCTCCAAAGC | |
| | CAAAGGGCAGCCCCGAGAACCACAGGTGTACACCCTGCCCCCATG | |
| | CCGGGAGGAGATGACCAAGAACCAGGTCAGCCTGTGGTGCCTGG | |
| | TCAAAGGCTTCTATCCCAGCGACATCGCCGTGGAGTGGGAGAGCA | |
| | ATGGGCAGCCGGAGAACAACTACAAGACCACGCCTCCCGTGCTG | |
| | GACTCCGACGGCTCCTTCTTCCTCTACAGCAAGCTCACCGTGGAC | |
| | AAGAGCAGGTGGCAGCAGGGGAACGTCTTCTCATGCTCCGTGATG | |
| | CATGAGGCTCTGCACAACCACTACACGCAGAAGAGCCTCTCCCTG | |
| | TCTCCGGGTAAA | |
| THC-S-PHC-Knob | GAAGTTCAACTTGTGGAGTCTGGAGGCGGTTTGGTACAACCTGGA | 491 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTAGTGTTTTCCCACTGGCTCCAGAAGTACAG | |
| | TTGGTGGAGTCTGGTGGAGGTCTTGTACAACCTGGCGGGAGTTTG | |
| | CGGCTTTCCTGCGCTGCAAGCGGGTTTACCTTCACCGATTATACCA | |
| | TGGATTGGGTACGCCAAGCCCCTGGTAAGTGCCTTGAGTGGGTTG | |
| | CCGATGTAAACCCTAATTCCGGAGGAAGTATCTATAATCAACGCT | |
| | TCAAGGGCCGATTCACTCTGAGTGTGGATCGAAGCAAGAACACCT | |
| | TGTACTTGCAGATGAATTCCTTGCGGGCTGAAGACACAGCCGTCT | |
| | ATTACTGCGCCCGAAATTTGGGGCCTTCATTCTATTTTGACTATTG | |
| | GGGTCAGGGAACTCTGGTAACTGTTTCAAGTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| TH-L-PH-Knob | GAAGTTCAACTTGTGGAGTCTGGAGGCGGTTTGGTACAACCTGGA | 492 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTAGTGTTTTCCCACTGGCTCCAGAAGTACAG | |
| | TTGGTGGAGTCTGGTGGAGGTCTTGTACAACCTGGCGGGAGTTTG | |
| | CGGCTTTCCTGCGCTGCAAGCGGGTTTACCTTCACCGATTATACCA | |
| | TGGATTGGGTACGCCAAGCCCCTGGTAAGGGCCTTGAGTGGGTTG | |
| | CCGATGTAAACCCTAATTCCGGAGGAAGTATCTATAATCAACGCT | |
| | TCAAGGGCCGATTCACTCTGAGTGTGGATCGAAGCAAGAACACCT | |
| | TGTACTTGCAGATGAATTCCTTGCGGGCTGAAGACACAGCCGTCT | |
| | ATTACTGCGCCCGAAATTTGGGGCCTTCATTCTATTTTGACTATTG | |
| | GGGTCAGGGAACTCTGGTAACTGTTTCAAGTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| THC-L-PH-Knob | GAAGTTCAACTTGTGGAGTCTGGAGGCGGTTTGGTACAACCTGGA | 493 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTAGTGTTTTCCCACTGGCTCCAGAAGTACAG | |
| | TTGGTGGAGTCTGGTGGAGGTCTTGTACAACCTGGCGGGAGTTTG | |
| | CGGCTTTCCTGCGCTGCAAGCGGGTTTACCTTCACCGATTATACCA | |
| | TGGATTGGGTACGCCAAGCCCCTGGTAAGGGCCTTGAGTGGGTTG | |
| | CCGATGTAAACCCTAATTCCGGAGGAAGTATCTATAATCAACGCT | |
| | TCAAGGGCCGATTCACTCTGAGTGTGGATCGAAGCAAGAACACCT | |
| | TGTACTTGCAGATGAATTCCTTGCGGGCTGAAGACACAGCCGTCT | |
| | ATTACTGCGCCCGAAATTTGGGGCCTTCATTCTATTTTGACTATTG | |
| | GGGTCAGGGAACTCTGGTAACTGTTTCAAGTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| TH-L-PHC-Knob | GAAGTTCAACTTGTGGAGTCTGGAGGCGGTTTGGTACAACCTGGA | 494 |
| | GGTTCACTTCGTTTGTCATGTGCTGCCTCTGGGTTTAATATCAAGG | |
| | ATACTTATATTCATTGGGTTCGCCAGGCACCCGGAAAAGGGCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTAGTGTTTTCCCACTGGCTCCAGAAGTACAG | |
| | TTGGTGGAGTCTGGTGGAGGTCTTGTACAACCTGGCGGGAGTTTG | |
| | CGGCTTTCCTGCGCTGCAAGCGGGTTTACCTTCACCGATTATACCA | |
| | TGGATTGGGTACGCCAAGCCCCTGGTAAGTGCCTTGAGTGGGTTG | |
| | CCGATGTAAACCCTAATTCCGGAGGAAGTATCTATAATCAACGCT | |
| | TCAAGGGCCGATTCACTCTGAGTGTGGATCGAAGCAAGAACACCT | |
| | TGTACTTGCAGATGAATTCCTTGCGGGCTGAAGACACAGCCGTCT | |
| | ATTACTGCGCCCGAAATTTGGGGCCTTCATTCTATTTTGACTATTG | |
| | GGGTCAGGGAACTCTGGTAACTGTTTCAAGTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| THC-L-PHC-Knob | GAAGTTCAACTTGTGGAGTCTGGAGGCGGTTTGGTACAACCTGGA | 495 |
| | GGTTCACTTCGTTTGTCATGTGCTGCCTCTGGGTTTAATATCAAGG | |
| | ATACTTATATTCATTGGGTTCGCCAGGCACCCGGAAAATGCCTGG | |
| | AATGGGTGGCCCGTATATATCCCACTAACGGTTACACTCGATATG | |
| | CTGACTCTGTAAAGGGTCGATTTACTATATCCGCTGATACTAGCAA | |
| | AAACACTGCCTACCTCCAGATGAACAGTCTGCGCGCCGAGGATAC | |
| | AGCAGTTTATTATTGTTCCCGATGGGGAGGTGACGGGTTCTATGCC | |
| | ATGGACTACTGGGGTCAAGGGACACTGGTAACCGTTTCTTCTGCT | |
| | AGCACAAAAGGACCTAGTGTTTTCCCACTGGCTCCAGAAGTACAG | |
| | TTGGTGGAGTCTGGTGGAGGTCTTGTACAACCTGGCGGGAGTTTG | |
| | CGGCTTTCCTGCGCTGCAAGCGGGTTTACCTTCACCGATTATACCA | |
| | TGGATTGGGTACGCCAAGCCCCTGGTAAGTGCCTTGAGTGGGTTG | |
| | CCGATGTAAACCCTAATTCCGGAGGAAGTATCTATAATCAACGCT | |
| | TCAAGGGCCGATTCACTCTGAGTGTGGATCGAAGCAAGAACACCT | |
| | TGTACTTGCAGATGAATTCCTTGCGGGCTGAAGACACAGCCGTCT | |
| | ATTACTGCGCCCGAAATTTGGGGCCTTCATTCTATTTTGACTATTG | |
| | GGGTCAGGGAACTCTGGTAACTGTTTCAAGTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| TL-S-PL-Knob | GACATTCAGATGACCCAATCCCCCTCTAGCCTTTCAGCCTCCGTAG | 496 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGACAGGGCACAAAAGTAGAGATTA | |
| | AGCGTACGGTGGCTGCTCCAGATATTCAAATGACCCAAAGTCCAA | |
| | GTTCCCTTTCAGCATCTGTCGGTGATCGAGTCACTATAACTTGTAA | |
| | GGCCAGCCAAGACGTTAGCATAGGAGTAGCATGGTACCAACAAA | |
| | AGCCTGGGAAGGCTCCCAAACTTCTCATTTATTCTGCTTCCTACCG | |
| | ATATACTGGTGTCCCAAGTAGATTTTCTGGCAGCGGATCTGGAAC | |
| | TGATTTTACATTGACTATCAGCTCCCTCCAGCCTGAGGACTTCGCT | |
| | ACTTATTACTGCCAACAGTACTACATTTATCCCTATACATTCGGTC | |
| | AAGGGACCAAAGTAGAGATCAAACGTACGGTGGCAGCTCCCAGC | |
| | GTTTTTATCTTTCCCCCATCCGACGAGCAGCTCAAGAGTGGCACTG | |
| | CCTCTGTAGTTTGTTTGCTGAATAACTTCTATCCACGTGAAGCAAA | |
| | AGTACAGTGGAAGGTCGATAATGCCCTTCAGAGCGGTAACAGCCA | |
| | AGAAAGTGTTACCGAGCAAGATTCCAAAGATTCCACTTACAGTCT | |
| | GTCCAGCACATTGACACTGAGTAAGGCTGATTACGAAAAACACAA | |
| | GGTGTACGCATGCGAGGTGACACACCAAGGTCTTTCATCTCCTGT | |
| | AACTAAGAGCTTTAACCGGGGAGAATGTGGTGGTGGGGGCAGCG | |
| | GGGGCGGAGGTAGTGGAGGCGGCGGTAGTGAACCAAAGAGTAGT | |
| | GACAAAACTCACACGTGCCCACCGTGCCCAGCACCTGAACTCCTG | |
| | GGGGGACCGTCAGTCTTCCTCTTCCCCCCAAAACCCAAGGACACC | |
| | CTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCA | |
| | CCAGGACTGGCTGAATGGCAAGGAGTACAAGTGCAAGGTCTCCA | |
| | ACAAAGCCCTCCCAGCCCCCATCGAGAAAACCATCTCCAAAGCCA | |
| | AAGGGCAGCCCCGAGAACCACAGGTGTACACCCTGCCCCCATGCC | |
| | GGGAGGAGATGACCAAGAACCAGGTCAGCCTGTGGTGCCTGGTC | |
| | AAAGGCTTCTATCCCAGCGACATCGCCGTGGAGTGGGAGAGCAAT | |
| | GGGCAGCCGGAGAACAACTACAAGACCACGCCTCCCGTGCTGGA | |
| | CTCCGACGGCTCCTTCTTCCTCTACAGCAAGCTCACCGTGGACAA | |
| | GAGCAGGTGGCAGCAGGGGAACGTCTTCTCATGCTCCGTGATGCA | |
| | TGAGGCTCTGCACAACCACTACACGCAGAAGAGCCTCTCCCTGTC | |
| | TCCGGGTAAA | |
| TLC-S-PL-Knob | GACATTCAGATGACCCAATCCCCCTCTAGCCTTTCAGCCTCCGTAG | 497 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGATGTGGCACAAAAGTAGAGATTA | |
| | AGCGTACGGTGGCTGCTCCAGATATTCAAATGACCCAAAGTCCAA | |
| | GTTCCCTTTCAGCATCTGTCGGTGATCGAGTCACTATAACTTGTAA | |
| | GGCCAGCCAAGACGTTAGCATAGGAGTAGCATGGTACCAACAAA | |
| | AGCCTGGGAAGGCTCCCAAACTTCTCATTTATTCTGCTTCCTACCG | |
| | ATATACTGGTGTCCCAAGTAGATTTTCTGGCAGCGGATCTGGAAC | |
| | TGATTTTACATTGACTATCAGCTCCCTCCAGCCTGAGGACTTCGCT | |
| | ACTTATTACTGCCAACAGTACTACATTTATCCCTATACATTCGGTC | |
| | AAGGGACCAAAGTAGAGATCAAACGTACGGTGGCAGCTCCCAGC | |
| | GTTTTTATCTTTCCCCCATCCGACGAGCAGCTCAAGAGTGGCACTG | |
| | CCTCTGTAGTTTGTTTGCTGAATAACTTCTATCCACGTGAAGCAAA | |
| | AGTACAGTGGAAGGTCGATAATGCCCTTCAGAGCGGTAACAGCCA | |
| | AGAAAGTGTTACCGAGCAAGATTCCAAAGATTCCACTTACAGTCT | |
| | GTCCAGCACATTGACACTGAGTAAGGCTGATTACGAAAAACACAA | |
| | GGTGTACGCATGCGAGGTGACACACCAAGGTCTTTCATCTCCTGT | |
| | AACTAAGAGCTTTAACCGGGGAGAATGTGGTGGTGGGGGCAGCG | |
| | GGGGCGGAGGTAGTGGAGGCGGCGGTAGTGAACCAAAGAGTAGT | |
| | GACAAAACTCACACGTGCCCACCGTGCCCAGCACCTGAACTCCTG | |
| | GGGGGACCGTCAGTCTTCCTCTTCCCCCCAAAACCCAAGGACACC | |
| | CTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCA | |
| | CCAGGACTGGCTGAATGGCAAGGAGTACAAGTGCAAGGTCTCCA | |
| | ACAAAGCCCTCCCAGCCCCCATCGAGAAAACCATCTCCAAAGCCA | |
| | AAGGGCAGCCCCGAGAACCACAGGTGTACACCCTGCCCCCATGCC | |
| | GGGAGGAGATGACCAAGAACCAGGTCAGCCTGTGGTGCCTGGTC | |
| | AAAGGCTTCTATCCCAGCGACATCGCCGTGGAGTGGGAGAGCAAT | |
| | GGGCAGCCGGAGAACAACTACAAGACCACGCCTCCCGTGCTGGA | |
| | CTCCGACGGCTCCTTCTTCCTCTACAGCAAGCTCACCGTGGACAA | |
| | GAGCAGGTGGCAGCAGGGGAACGTCTTCTCATGCTCCGTGATGCA | |
| | TGAGGCTCTGCACAACCACTACACGCAGAAGAGCCTCTCCCTGTC | |
| | TCCGGGTAAA | |
| TL-S-PLC-Knob | GACATTCAGATGACCCAATCCCCCTCTAGCCTTTCAGCCTCCGTAG | 498 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGACAGGGCACAAAAGTAGAGATTA | |
| | AGCGTACGGTGGCTGCTCCAGATATTCAAATGACCCAAAGTCCAA | |
| | GTTCCCTTTCAGCATCTGTCGGTGATCGAGTCACTATAACTTGTAA | |
| | GGCCAGCCAAGACGTTAGCATAGGAGTAGCATGGTACCAACAAA | |
| | AGCCTGGGAAGGCTCCCAAACTTCTCATTTATTCTGCTTCCTACCG | |
| | ATATACTGGTGTCCCAAGTAGATTTTCTGGCAGCGGATCTGGAAC | |
| | TGATTTTACATTGACTATCAGCTCCCTCCAGCCTGAGGACTTCGCT | |
| | ACTTATTACTGCCAACAGTACTACATTTATCCCTATACATTCGGTT | |
| | GTGGGACCAAAGTAGAGATCAAACGTACGGTGGCAGCTCCCAGC | |
| | GTTTTTATCTTTCCCCCATCCGACGAGCAGCTCAAGAGTGGCACTG | |
| | CCTCTGTAGTTTGTTTGCTGAATAACTTCTATCCACGTGAAGCAAA | |
| | AGTACAGTGGAAGGTCGATAATGCCCTTCAGAGCGGTAACAGCCA | |
| | AGAAAGTGTTACCGAGCAAGATTCCAAAGATTCCACTTACAGTCT | |
| | GTCCAGCACATTGACACTGAGTAAGGCTGATTACGAAAAACACAA | |
| | GGTGTACGCATGCGAGGTGACACACCAAGGTCTTTCATCTCCTGT | |
| | AACTAAGAGCTTTAACCGGGGAGAATGTGGTGGTGGGGGCAGCG | |
| | GGGGCGGAGGTAGTGGAGGCGGCGGTAGTGAACCAAAGAGTAGT | |
| | GACAAAACTCACACGTGCCCACCGTGCCCAGCACCTGAACTCCTG | |
| | GGGGGACCGTCAGTCTTCCTCTTCCCCCCAAAACCCAAGGACACC | |
| | CTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCA | |
| | CCAGGACTGGCTGAATGGCAAGGAGTACAAGTGCAAGGTCTCCA | |
| | ACAAAGCCCTCCCAGCCCCCATCGAGAAAACCATCTCCAAAGCCA | |
| | AAGGGCAGCCCCGAGAACCACAGGTGTACACCCTGCCCCCATGCC | |
| | GGGAGGAGATGACCAAGAACCAGGTCAGCCTGTGGTGCCTGGTC | |
| | AAAGGCTTCTATCCCAGCGACATCGCCGTGGAGTGGGAGAGCAAT | |
| | GGGCAGCCGGAGAACAACTACAAGACCACGCCTCCCGTGCTGGA | |
| | CTCCGACGGCTCCTTCTTCCTCTACAGCAAGCTCACCGTGGACAA | |
| | GAGCAGGTGGCAGCAGGGGAACGTCTTCTCATGCTCCGTGATGCA | |
| | TGAGGCTCTGCACAACcactacACGCAGAAGAGCCTCTCCCTGTCT | |
| | CCGGGTAAA | |
| TLC-S-PLC-Knob | GACATTCAGATGACCCAATCCCCCTCTAGCCTTTCAGCCTCCGTAG | 499 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGATGTGGCACAAAAGTAGAGATTA | |
| | AGCGTACGGTGGCTGCTCCAGATATTCAAATGACCCAAAGTCCAA | |
| | GTTCCCTTTCAGCATCTGTCGGTGATCGAGTCACTATAACTTGTAA | |
| | GGCCAGCCAAGACGTTAGCATAGGAGTAGCATGGTACCAACAAA | |
| | AGCCTGGGAAGGCTCCCAAACTTCTCATTTATTCTGCTTCCTACCG | |
| | ATATACTGGTGTCCCAAGTAGATTTTCTGGCAGCGGATCTGGAAC | |
| | TGATTTTACATTGACTATCAGCTCCCTCCAGCCTGAGGACTTCGCT | |
| | ACTTATTACTGCCAACAGTACTACATTTATCCCTATACATTCGGTT | |
| | GTGGGACCAAAGTAGAGATCAAACGTACGGTGGCAGCTCCCAGC | |
| | GTTTTTATCTTTCCCCCATCCGACGAGCAGCTCAAGAGTGGCACTG | |
| | CCTCTGTAGTTTGTTTGCTGAATAACTTCTATCCACGTGAAGCAAA | |
| | AGTACAGTGGAAGGTCGATAATGCCCTTCAGAGCGGTAACAGCCA | |
| | AGAAAGTGTTACCGAGCAAGATTCCAAAGATTCCACTTACAGTCT | |
| | GTCCAGCACATTGACACTGAGTAAGGCTGATTACGAAAAACACAA | |
| | GGTGTACGCATGCGAGGTGACACACCAAGGTCTTTCATCTCCTGT | |
| | AACTAAGAGCTTTAACCGGGGAGAATGTGGTGGTGGGGGCAGCG | |
| | GGGGCGGAGGTAGTGGAGGCGGCGGTAGTGAACCAAAGAGTAGT | |
| | GACAAAACTCACACGTGCCCACCGTGCCCAGCACCTGAACTCCTG | |
| | GGGGGACCGTCAGTCTTCCTCTTCCCCCCAAAACCCAAGGACACC | |
| | CTCATGATCTCCCGGACCCCTGAGGTCACATGCGTGGTGGTGGAC | |
| | GTGAGCCACGAAGACCCTGAGGTCAAGTTCAACTGGTACGTGGAC | |
| | GGCGTGGAGGTGCATAATGCCAAGACAAAGCCGCGGGAGGAGCA | |
| | GTACAACAGCACGTACCGTGTGGTCAGCGTCCTCACCGTCCTGCA | |
| | CCAGGACTGGCTGAATGGCAAGGAGTACAAGTGCAAGGTCTCCA | |
| | ACAAAGCCCTCCCAGCCCCCATCGAGAAAACCATCTCCAAAGCCA | |
| | AAGGGCAGCCCCGAGAACCACAGGTGTACACCCTGCCCCCATGCC | |
| | GGGAGGAGATGACCAAGAACCAGGTCAGCCTGTGGTGCCTGGTC | |
| | AAAGGCTTCTATCCCAGCGACATCGCCGTGGAGTGGGAGAGCAAT | |
| | GGGCAGCCGGAGAACAACTACAAGACCACGCCTCCCGTGCTGGA | |
| | CTCCGACGGCTCCTTCTTCCTCTACAGCAAGCTCACCGTGGACAA | |
| | GAGCAGGTGGCAGCAGGGGAACGTCTTCTCATGCTCCGTGATGCA | |
| | TGAGGCTCTGCACAACCACTACACGCAGAAGAGCCTCTCCCTGTC | |
| | TCCGGGTAAA | |
| TL-L-PL-Knob | GACATTCAGATGACCCAATCCCCCTCTAGCCTTTCAGCCTCCGTAG | 500 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGACAGGGCACAAAAGTAGAGATTA | |
| | AGCGTACGGTGGCTGCTCCATCCGTTTTTATCTTTCCCCCAGATAT | |
| | TCAAATGACCCAAAGTCCAAGTTCCCTTTCAGCATCTGTCGGTGAT | |
| | CGAGTCACTATAACTTGTAAGGCCAGCCAAGACGTTAGCATAGGA | |
| | GTAGCATGGTACCAACAAAAGCCTGGGAAGGCTCCCAAACTTCTC | |
| | ATTTATTCTGCTTCCTACCGATATACTGGTGTCCCAAGTAGATTTT | |
| | CTGGCAGCGGATCTGGAACTGATTTTACATTGACTATCAGCTCCCT | |
| | CCAGCCTGAGGACTTCGCTACTTATTACTGCCAACAGTACTACATT | |
| | TATCCCTATACATTCGGTCAAGGGACCAAAGTAGAGATCAAACGT | |
| | ACGGTGGCAGCTCCCAGCGTTTTTATCTTTCCCCCATCCGACGAGC | |
| | AGCTCAAGAGTGGCACTGCCTCTGTAGTTTGTTTGCTGAATAACTT | |
| | CTATCCACGTGAAGCAAAAGTACAGTGGAAGGTCGATAATGCCCT | |
| | TCAGAGCGGTAACAGCCAAGAAAGTGTTACCGAGCAAGATTCCA | |
| | AAGATTCCACTTACAGTCTGTCCAGCACATTGACACTGAGTAAGG | |
| | CTGATTACGAAAAACACAAGGTGTACGCATGCGAGGTGACACAC | |
| | CAAGGTCTTTCATCTCCTGTAACTAAGAGCTTTAACCGGGGAGAA | |
| | TGTGGTGGTGGGGGCAGCGGGGGCGGAGGTAGTGGAGGCGGCGG | |
| | TAGTGAACCAAAGAGTAGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAA | |
| | GTTCAACTGGTACGTGGACGGCGTGGAGGTGCATAATGCCAAGAC | |
| | AAAGCCGCGGGAGGAGCAGTACAACAGCACGTACCGTGTGGTCA | |
| | GCGTCCTCACCGTCCTGCACCAGGACTGGCTGAATGGCAAGGAGT | |
| | ACAAGTGCAAGGTCTCCAACAAAGCCCTCCCAGCCCCCATCGAGA | |
| | AAACCATCTCCAAAGCCAAAGGGCAGCCCCGAGAACCACAGGTG | |
| | TACACCCTGCCCCCATGCCGGGAGGAGATGACCAAGAACCAGGTC | |
| | AGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAG | |
| | CAAGCTCACCGTGGACAAGAGCAGGTGGCAGCAGGGGAACGTCTT | |
| | CTCATGCTCCGTGATGCATGAGGCTCTGCACAACCACTACACGCA | |
| | GAAGAGCCTCTCCCTGTCTCCGGGTAAA | |
| TLC-L-PL-Knob | GACATTCAGATGACCCAATCCCCCTCTAGCCTTTCAGCCTCCGTAG | 501 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGATGTGGCACAAAAGTAGAGATTA | |
| | AGCGTACGGTGGCTGCTCCATCCGTTTTTATCTTTCCCCCAGATAT | |
| | TCAAATGACCCAAAGTCCAAGTTCCCTTTCAGCATCTGTCGGTGAT | |
| | CGAGTCACTATAACTTGTAAGGCCAGCCAAGACGTTAGCATAGGA | |
| | GTAGCATGGTACCAACAAAAGCCTGGGAAGGCTCCCAAACTTCTC | |
| | ATTTATTCTGCTTCCTACCGATATACTGGTGTCCCAAGTAGATTTT | |
| | CTGGCAGCGGATCTGGAACTGATTTTACATTGACTATCAGCTCCCT | |
| | CCAGCCTGAGGACTTCGCTACTTATTACTGCCAACAGTACTACATT | |
| | TATCCCTATACATTCGGTCAAGGGACCAAAGTAGAGATCAAACGT | |
| | ACGGTGGCAGCTCCCAGCGTTTTTATCTTTCCCCCATCCGACGAGC | |
| | AGCTCAAGAGTGGCACTGCCTCTGTAGTTTGTTTGCTGAATAACTT | |
| | CTATCCACGTGAAGCAAAAGTACAGTGGAAGGTCGATAATGCCCT | |
| | TCAGAGCGGTAACAGCCAAGAAAGTGTTACCGAGCAAGATTCCA | |
| | AAGATTCCACTTACAGTCTGTCCAGCACATTGACACTGAGTAAGG | |
| | CTGATTACGAAAAACACAAGGTGTACGCATGCGAGGTGACACAC | |
| | CAAGGTCTTTCATCTCCTGTAACTAAGAGCTTTAACCGGGGAGAA | |
| | TGTGGTGGTGGGGGCAGCGGGGGCGGAGGTAGTGGAGGCGGCGG | |
| | TAGTGAACCAAAGAGTAGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAA | |
| | GTTCAACTGGTACGTGGACGGCGTGGAGGTGCATAATGCCAAGAC | |
| | AAAGCCGCGGGAGGAGCAGTACAACAGCACGTACCGTGTGGTCA | |
| | GCGTCCTCACCGTCCTGCACCAGGACTGGCTGAATGGCAAGGAGT | |
| | ACAAGTGCAAGGTCTCCAACAAAGCCCTCCCAGCCCCCATCGAGA | |
| | AAACCATCTCCAAAGCCAAAGGGCAGCCCCGAGAACCACAGGTG | |
| | TACACCCTGCCCCCATGCCGGGAGGAGATGACCAAGAACCAGGTC | |
| | AGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTT | |
| | CTCATGCTCCGTGATGCATGAGGCTCTGCACAACCACTACACGCA | |
| | GAAGAGCCTCTCCCTGTCTCCGGGTAAA | |
| TL-L-PLC-Knob | GACATTCAGATGACCCAATCCCCCTCTAGCCTTTCAGCCTCCGTAG | 502 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGACAGGGCACAAAAGTAGAGATTA | |
| | AGCGTACGGTGGCTGCTCCATCCGTTTTTATCTTTCCCCCAGATAT | |
| | TCAAATGACCCAAAGTCCAAGTTCCCTTTCAGCATCTGTCGGTGAT | |
| | CGAGTCACTATAACTTGTAAGGCCAGCCAAGACGTTAGCATAGGA | |
| | GTAGCATGGTACCAACAAAAGCCTGGGAAGGCTCCCAAACTTCTC | |
| | ATTTATTCTGCTTCCTACCGATATACTGGTGTCCCAAGTAGATTTT | |
| | CTGGCAGCGGATCTGGAACTGATTTTACATTGACTATCAGCTCCCT | |
| | CCAGCCTGAGGACTTCGCTACTTATTACTGCCAACAGTACTACATT | |
| | TATCCCTATACATTCGGTTGTGGGACCAAAGTAGAGATCAAACGT | |
| | ACGGTGGCAGCTCCCAGCGTTTTTATCTTTCCCCCATCCGACGAGC | |
| | AGCTCAAGAGTGGCACTGCCTCTGTAGTTTGTTTGCTGAATAACTT | |
| | CTATCCACGTGAAGCAAAAGTACAGTGGAAGGTCGATAATGCCCT | |
| | TCAGAGCGGTAACAGCCAAGAAAGTGTTACCGAGCAAGATTCCA | |
| | AAGATTCCACTTACAGTCTGTCCAGCACATTGACACTGAGTAAGG | |
| | CTGATTACGAAAAACACAAGGTGTACGCATGCGAGGTGACACAC | |
| | CAAGGTCTTTCATCTCCTGTAACTAAGAGCTTTAACCGGGGAGAA | |
| | TGTGGTGGTGGGGGCAGCGGGGGCGGAGGTAGTGGAGGCGGCGG | |
| | TAGTGAACCAAAGAGTAGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAA | |
| | GTTCAACTGGTACGTGGACGGCGTGGAGGTGCATAATGCCAAGAC | |
| | AAAGCCGCGGGAGGAGCAGTACAACAGCACGTACCGTGTGGTCA | |
| | GCGTCCTCACCGTCCTGCACCAGGACTGGCTGAATGGCAAGGAGT | |
| | ACAAGTGCAAGGTCTCCAACAAAGCCCTCCCAGCCCCCATCGAGA | |
| | AAACCATCTCCAAAGCCAAAGGGCAGCCCCGAGAACCACAGGTG | |
| | TACACCCTGCCCCCATGCCGGGAGGAGATGACCAAGAACCAGGTC | |
| | AGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAGC | |
| | AAGCTCACCGTGGACAAGAGCAGGTGGCAGCAGGGGAACGTCTT | |
| | CTCATGCTCCGTGATGCATGAGGCTCTGCACAACCACTACACGCA | |
| | GAAGAGCCTCTCCCTGTCTCCGGGTAAA | |
| TLC-L-PLC-Knob | GACATTCAGATGACCCAATCCCCCTCTAGCCTTTCAGCCTCCGTAG | 503 |
| | GGGACAGAGTTACAATAACCTGTAGGGCTAGTCAGGACGTTAATA | |
| | CAGCAGTCGCATGGTACCAGCAGAAGCCCGGTAAGGCTCCTAAGCT | |
| | TTTGATCTACAGTGCTTCTTTCCTGTATTCTGGTGTACCTAGCCGA | |
| | TTCTCAGGCTCTCGGAGTGGCACAGACTTCACTCTGACAATTTCAA | |
| | GTCTCCAGCCAGAAGATTTTGCAACCTACTACTGCCAACAACACT | |
| | ACACTACTCCTCCAACCTTCGGATGTGGCACAAAAGTAGAGATTA | |
| | AGCGTACGGTGGCTGCTCCATCCGTTTTTATCTTTCCCCCAGATAT | |
| | TCAAATGACCCAAAGTCCAAGTTCCCTTTCAGCATCTGTCGGTGAT | |
| | CGAGTCACTATAACTTGTAAGGCCAGCCAAGACGTTAGCATAGGA | |
| | GTAGCATGGTACCAACAAAAGCCTGGGAAGGCTCCCAAACTTCTC | |
| | ATTTATTCTGCTTCCTACCGATATACTGGTGTCCCAAGTAGATTTT | |
| | CTGGCAGCGGATCTGGAACTGATTTTACATTGACTATCAGCTCCCT | |
| | CCAGCCTGAGGACTTCGCTACTTATTACTGCCAACAGTACTACATT | |
| | TATCCCTATACATTCGGTTGTGGGACCAAAGTAGAGATCAAACGT | |
| | ACGGTGGCAGCTCCCAGCGTTTTTATCTTTCCCCCATCCGACGAGC | |
| | AGCTCAAGAGTGGCACTGCCTCTGTAGTTTGTTTGCTGAATAACTT | |
| | CTATCCACGTGAAGCAAAAGTACAGTGGAAGGTCGATAATGCCCT | |
| | TCAGAGCGGTAACAGCCAAGAAAGTGTTACCGAGCAAGATTCCA | |
| | AAGATTCCACTTACAGTCTGTCCAGCACATTGACACTGAGTAAGG | |
| | CTGATTACGAAAAACACAAGGTGTACGCATGCGAGGTGACACAC | |
| | CAAGGTCTTTCATCTCCTGTAACTAAGAGCTTTAACCGGGGAGAA | |
| | TGTGGTGGTGGGGGCAGCGGGGGCGGAGGTAGTGGAGGCGGCGG | |
| | TAGTGAACCAAAGAGTAGTGACAAAACTCACACGTGCCCACCGTG | |
| | CCCAGCACCTGAACTCCTGGGGGGACCGTCAGTCTTCCTCTTCCCC | |
| | CCAAAACCCAAGGACACCCTCATGATCTCCCGGACCCCTGAGGTC | |
| | ACATGCGTGGTGGTGGACGTGAGCCACGAAGACCCTGAGGTCAA | |
| | GTTCAACTGGTACGTGGACGGCGTGGAGGTGCATAATGCCAAGAC | |
| | AAAGCCGCGGGAGGAGCAGTACAACAGCACGTACCGTGTGGTCA | |
| | GCGTCCTCACCGTCCTGCACCAGGACTGGCTGAATGGCAAGGAGT | |
| | ACAAGTGCAAGGTCTCCAACAAAGCCCTCCCAGCCCCCATCGAGA | |
| | AAACCATCTCCAAAGCCAAAGGGCAGCCCCGAGAACCACAGGTG | |
| | TACACCCTGCCCCCATGCCGGGAGGAGATGACCAAGAACCAGGTC | |
| | AGCCTGTGGTGCCTGGTCAAAGGCTTCTATCCCAGCGACATCGCC | |
| | GTGGAGTGGGAGAGCAATGGGCAGCCGGAGAACAACTACAAGAC | |
| | CACGCCTCCCGTGCTGGACTCCGACGGCTCCTTCTTCCTCTACAG | |
| | CAAGCTCACCGTGGACAAGAGCAGGTGGCAGCAGGGGAACGTCTT | |
| | CTCATGCTCCGTGATGCATGAGGCTCTGCACAACCACTACACGCA | |
| | GAAGAGCCTCTCCCTGTCTCCGGGTAAA | |

TABLE 34

| | VH1-linker-VH2- | VL1-linker-VL2- | |
|---|---|---|---|
| | CH1-Fc(Knob) | CL-linker-Fc(Knob) | Fc(Hole) |
| HP501 | SEQ ID NO: 51 | SEQ ID NO: 59 | SEQ ID NO: 7 |
| HP502 | SEQ ID NO: 52 | SEQ ID NO: 60 | SEQ ID NO: 7 |
| HP503 | SEQ ID NO: 53 | SEQ ID NO: 61 | SEQ ID NO: 7 |
| HP504 | SEQ ID NO: 54 | SEQ ID NO: 62 | SEQ ID NO: 7 |
| HP505 | SEQ ID NO: 51 | SEQ ID NO: 63 | SEQ ID NO: 7 |
| HP506 | SEQ ID NO: 52 | SEQ ID NO: 64 | SEQ ID NO: 7 |
| HP507 | SEQ ID NO: 53 | SEQ ID NO: 65 | SEQ ID NO: 7 |
| HP508 | SEQ ID NO: 54 | SEQ ID NO: 66 | SEQ ID NO: 7 |
| HP509 | SEQ ID NO: 55 | SEQ ID NO: 59 | SEQ ID NO: 7 |
| HP510 | SEQ ID NO: 56 | SEQ ID NO: 60 | SEQ ID NO: 7 |
| HP511 | SEQ ID NO: 57 | SEQ ID NO: 61 | SEQ ID NO: 7 |
| HP512 | SEQ ID NO: 58 | SEQ ID NO: 62 | SEQ ID NO: 7 |
| HP513 | SEQ ID NO: 55 | SEQ ID NO: 63 | SEQ ID NO: 7 |
| HP514 | SEQ ID NO: 56 | SEQ ID NO: 64 | SEQ ID NO: 7 |
| HP515 | SEQ ID NO: 57 | SEQ ID NO: 65 | SEQ ID NO: 7 |
| HP516 | SEQ ID NO: 58 | SEQ ID NO: 66 | SEQ ID NO: 7 |

Purity was analyzed by size exclusion chromatography in the same manner as described in Example 1 (FIG. 27, Table 35). The analysis showed that HP503, HP507, HP511, and HP515, in which the first V domain is wild type and the second V domain is Cys substituted variant (VH 44C, VL 100C), have an excellent purity (FIG. 27, Table 35).

TABLE 35

| Clone name | Monomer purity (%) |
|---|---|
| HP501 | 93.06 |
| HP502 | 94.91 |
| HP503 | 96.45 |
| HP504 | 86.77 |
| HP505 | 92.94 |
| HP506 | 82.89 |
| HP507 | 97.31 |
| HP508 | 57.51 |
| HP509 | 90.88 |
| HP510 | 86.56 |
| HP511 | 96.95 |
| HP512 | 68.52 |
| HP513 | 89.89 |
| HP514 | 79.31 |
| HP515 | 96.20 |
| HP516 | 58.07 |

The binding constants of HP501, HP502, HP503, HP504, HP505, HP506, HP507, HP508, HP509, HP510, HP511, HP512, HP513, HP514, HP515, and HP516 to D2 region and D4 region in HER2 protein were determined using the Octet Red96e (Sartorius). In order to analyze the binding constants of the sixteen antibodies to the D2 region, the human HER2 recombinant protein (R&D systems, 1129-ER) was loaded onto the Anti-Penta-HIS (HIS1K) biosensor (Sartorius, 18-5120) and then saturated with 100 nM trastuzumab which targets the D4 region. Thereafter, the sixteen antibodies were added in a binding reaction (300 seconds) and a dissociation reaction (600 seconds) at a concentration of 100 nM, and their affinities for the D2 region were calculated (Table 36). In order to analyze the binding constants of the sixteen antibodies to the D4 region, the human HER2 recombinant protein (R&D systems, 1129-ER) was loaded onto the Anti-Penta-HIS (HIS1K) biosensor (Sartorius, 18-5120), and then saturated with 100 nM pertuzumab which targets the D2 region. Thereafter, the sixteen antibodies were added in a binding reaction (300 seconds) and a dissociation reaction (600 seconds) at a concentration of 100 nM, and their affinities for the D4 region were calculated (Table 36). The binding constants of HP507, HP511, and HP515 to D2 region were 2.285, 3.267, and 2.012 nM, respectively, showing excellent binding affinities to the D2 region compared to other clones (Table 36). HP503 has a binding constant of 8.098 nM to the D2 region and shows a relatively low binding ability to the D2 region compared to HP507, HP511, and HP515. However, it was found that when measuring the binding constant to the D4 region, the binding constants of HP503, HP507, HP511, and HP515 were 0.181, 0.228, 0.162, and 0.227 nM, respectively, demonstrating strong binding affinity Table 36).

TABLE 36

| 100 nM | | | | | | |
|---|---|---|---|---|---|---|
| Single | Binding kinetics of mAbs to D2 epitope | | | Binding kinetics of mAbs to D4 epitope | | |
| Kinetics | K_{D} (nM) | Kₐ (1/Ms) | K_{d} (1/s) | K_{D} (nM) | Kₐ (1/Ms) | K_{d} (1/s) |
| HP501 | 13.009 | 8.05E+03 | 1.05E−04 | 0.218 | 2.00E+05 | 4.36E−05 |
| HP502 | 8.433 | 8.97E+03 | 7.56E−05 | 0.531 | 1.47E+05 | 7.82E−05 |
| HP503 | 8.098 | 4.69E+03 | 3.80E−05 | 0.181 | 2.14E+05 | 3.87E−05 |
| HP504 | 7.304 | 2.53E+04 | 1.85E−04 | 0.788 | 1.13E+05 | 8.94E−05 |
| HP505 | 5.882 | 1.41E+04 | 8.30E−05 | 0.586 | 1.50E+05 | 8.79E−05 |
| HP506 | 5.682 | 1.21E+04 | 6.89E−05 | 0.611 | 1.37E+05 | 8.37E−05 |
| HP507 | 2.285 | 1.85E+04 | 4.23E−05 | 0.228 | 1.99E+05 | 4.53E−05 |
| HP508 | 6.490 | 2.36E+04 | 1.53E−04 | 1.219 | 8.44E+04 | 1.03E−04 |
| HP509 | 7.962 | 1.33E+04 | 1.06E−04 | 0.393 | 1.72E+05 | 6.77E−05 |
| HP510 | 10.736 | 9.86E+03 | 1.06E−04 | 0.336 | 1.97E+05 | 6.60E−05 |
| HP511 | 3.267 | 1.47E+04 | 4.81E−05 | 0.162 | 2.27E+05 | 3.67E−05 |
| HP512 | 7.615 | 2.80E+04 | 2.13E−04 | 0.667 | 1.12E+05 | 7.47E−05 |
| HP513 | 4.739 | 2.17E+04 | 1.03E−04 | 0.381 | 1.95E+05 | 7.43E−05 |
| HP514 | 5.920 | 1.78E+04 | 1.06E−04 | 0.451 | 1.74E+05 | 7.82E−05 |
| HP515 | 2.012 | 2.89E+04 | 5.81E−05 | 0.227 | 2.14E+05 | 4.87E−05 |
| HP516 | 5.094 | 3.29E+04 | 1.67E−04 | 0.607 | 1.23E+05 | 7.46E−05 |

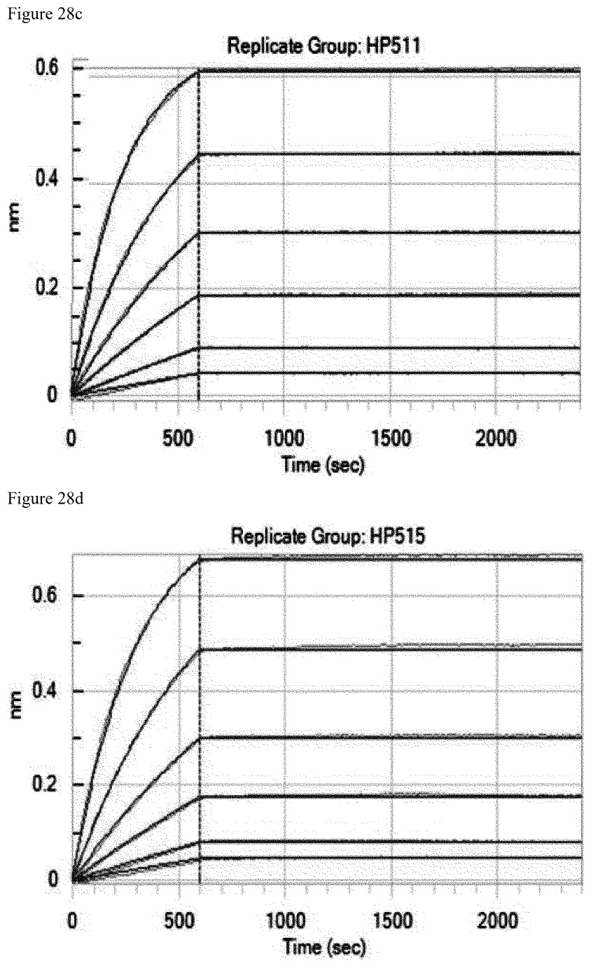

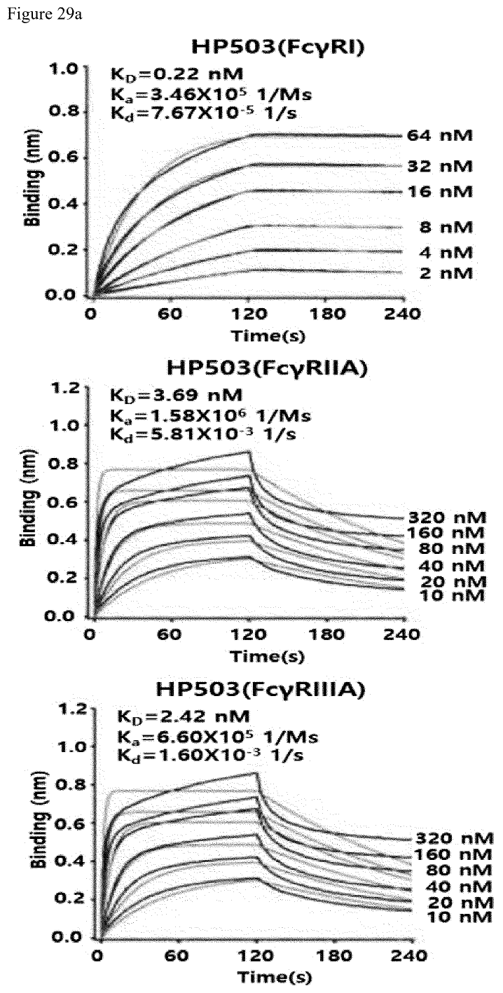

The binding constants of HP503, HP507, HP511, and P515 to the HER2 extracellular domain (ECD) were measured using Octet Red96e (Sartorius). The human HER2 recombinant protein (R&D systems, 1129-ER) was loaded onto the Anti-Penta-HIS (HIS1K) biosensor (Sartorius, 18-5120). HP503 or P57 or P5 or) P515 at a concentration of 0.25, 0.5, 1, 2, 4, or 8 nM were added in a binding reaction (600 seconds) and a dissociation reaction (1,800 seconds) in the sensor loaded with the HER2 protein, and the binding constants were calculated (FIGS. 28a to 28d, Table 37). It was found that HP507, HP511, and HP515 had a dissociation constant (<1.0E-07 1/s) that exceeded the measurement limit of the equipment under the following analysis conditions (FIGS. 28a to 28d, Table 37).

TABLE 37

| | K_{D} (nM) | Kₐ (1/Ms) | K_{d} (1/s) |
|---|---|---|---|
| HP503 | 6.57E−12 | 4.69E+05 | 3.08E−06 |
| HP507 | <1.0E−12 | 4.58E+05 | <1.0E−07 |
| HP511 | <1.0E−12 | 5.20E+05 | <1.0E−07 |
| HP515 | <1.0E−12 | 4.14E+05 | <1.0E−07 |

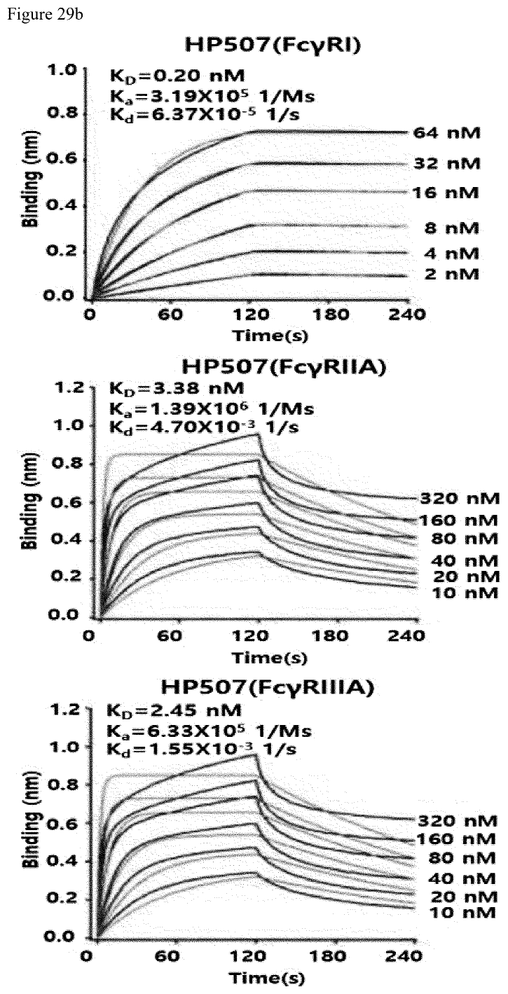

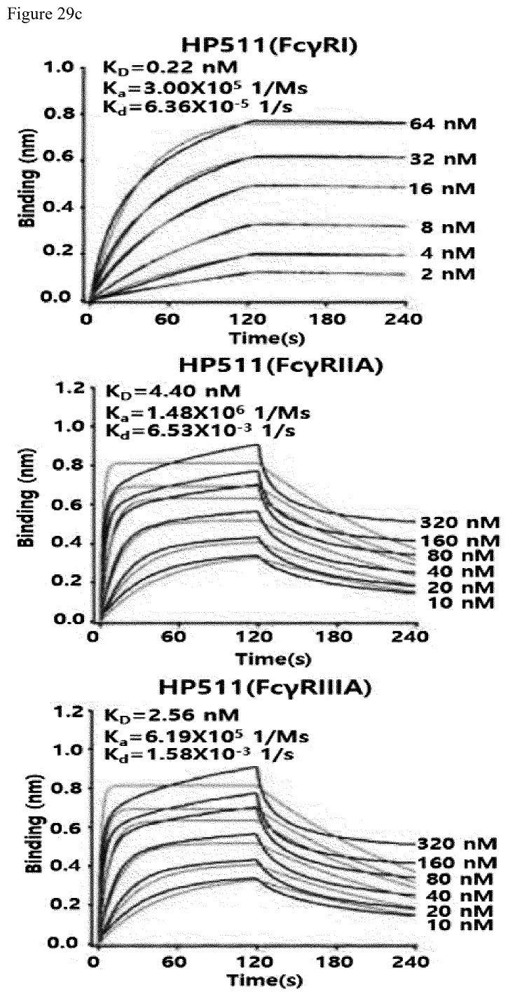

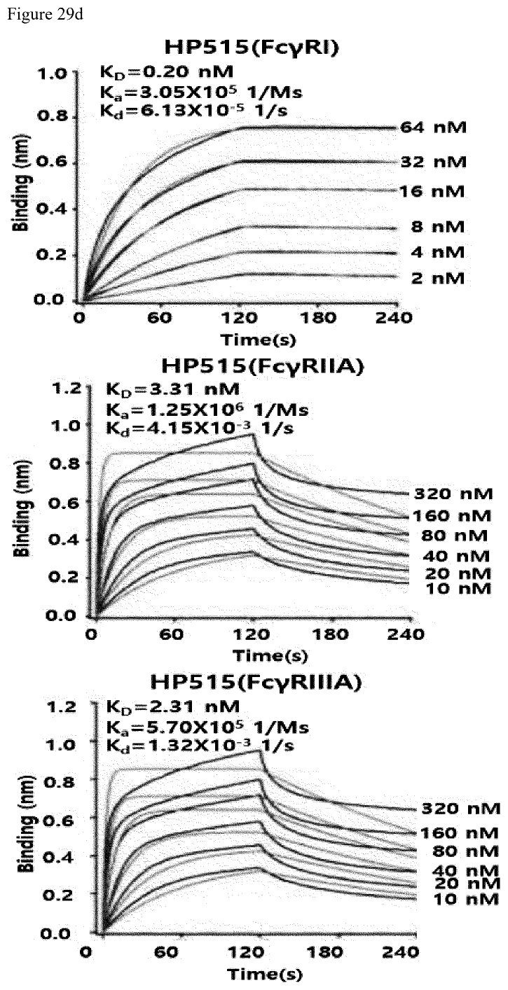

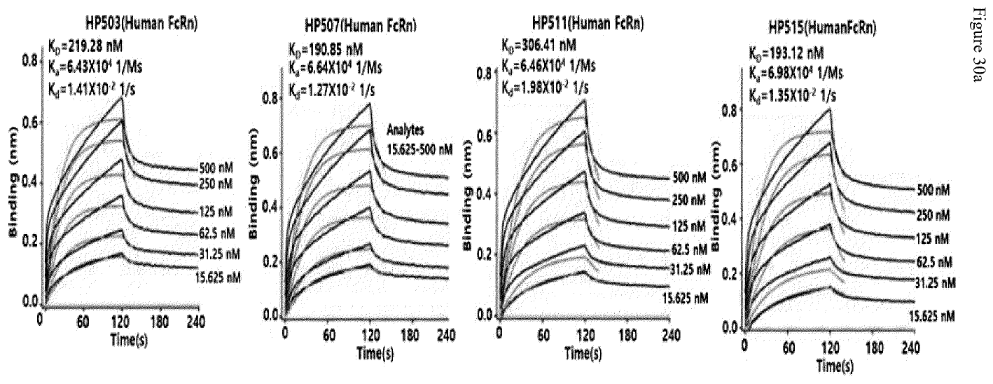

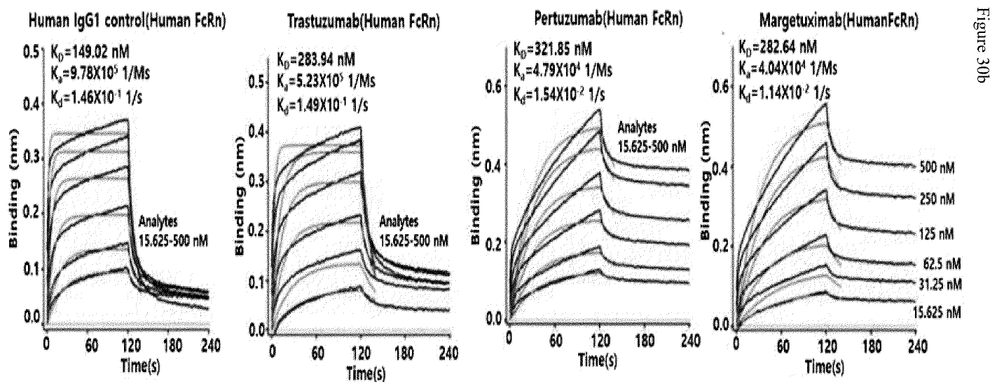

The measured binding constants of HP503, HP507, HP511, and BP515 to Fcγ receptors were analyzed using Octet Red96e (Sartorius) in the same manner as described in Example 10 (FIGS. 29a to 29d, Table 38). The analysis showed that HP503, HP507, HP511, and BP515 have excellent binding affinities to FcγRI (CD64), FcγRIIA (CD32A, 131R), and FcγRIIIA (CD16A, 176V) compared to human IgG1, trastuzumab, pertuzumab, and margetuximab (FIGS. 21a to 21h, FIGS. 22a to 22h, FIGS. 23a to 23h, FIGS. 29a to 29d, and Tables 29 and 38).

TABLE 38

| | FcγRI (CD64) | | | FcγRIIA (CD32A, 131R) | | | FcγRIIIA (CD16A, 176V) | | |
|---|---|---|---|---|---|---|---|---|---|
| Antigen | KD | Ka | Kd | KD | Ka | Kd | KD | Ka | Kd |
| Antibody | (nM) | (1/Ms) | (1/s) | (nM) | (1/Ms) | (1/s) | (nM) | (1/Ms) | (1/s) |
| HP503 | 0.22 | 3.46E+05 | 7.67E−05 | 3.69 | 1.58E+06 | 5.81E−03 | 2.42 | 6.60E+05 | 1.60E−03 |
| HP507 | 0.20 | 3.19E+05 | 6.37E−05 | 3.38 | 1.39E+06 | 4.70E−03 | 2.45 | 6.33E+05 | 1.55E−03 |
| HP511 | 0.22 | 3.00E+05 | 6.63E−05 | 4.40 | 1.48E+06 | 6.53E−03 | 2.56 | 6.19E+05 | 1.58E−03 |
| HP515 | 0.20 | 3.05E+05 | 6.13E−05 | 13.31 | 1.25E+06 | 4.15E−03 | 2.31 | 5.70E+05 | 1.32E−03 |

The binding constants of HP503, HP507, BP511, and BP515 to the neonatal Fc receptor (FcRn) were measured using Octet Red96e (Sartorius). HP503, HP507, BP511, HP515, human IgG1 (Bio X cell, BE0297), trastuzumab, pertuzumab, and margetuximab were loaded onto the anti-human Fab-CH1 2nd generation (FAB2G) biosensor (Sartorius, 18-5126), and a binding and a dissociation time were set to be 120 seconds, respectively (FIGS. 30a to 30b, Table 39). For analysis, Kinetics Buffer (Sartorius, 18-1105) was used at pH 6.0.

TABLE 39

| Antibodies | K_{D} (nM) | Kₐ (1/Ms) | K_{d} (1/s) |
|---|---|---|---|
| HP503 | 219.28 | 6.43E+04 | 1.41E−02 |
| HP507 | 190.85 | 6.64E+04 | 1.27E−02 |
| HP511 | 306.41 | 6.46E+04 | 1.98E−02 |
| HP515 | 193.12 | 6.98E+04 | 1.35E−02 |
| Human IgG1 | 149.02 | 9.78E+05 | 1.46E−01 |
| Trastuzumab | 283.94 | 5.23E+05 | 1.49E−01 |
| Pertuzumab | 321.85 | 4.79E+04 | 1.54E−02 |
| Margetuximab | 282.64 | 4.04E+04 | 1.14E−02 |

### Example 13. Analysis of Complement-Dependent Cytotoxicity

For the analysis of complement-dependent cytotoxicity, BT474 (HER2 3+; high) breast cancer cell line and NCI-N87 (HER2 3+; high) gastric cancer cell line were used. The cells were diluted in cell culture medium and dispensed in a 96-well plate at 10,000 cells per well. The cells, antibodies, and human serum (Sigma, H4522) were each reacted at a volume ratio of 1:1:1.

In order to identify a dose-response relationship, human IgG1, trastuzumab (TRA), trastuzumab+pertuzumab (TRA+PER), H01, and H01+P01 were serially diluted by a factor of two six times from initial concentration of 1200 nM, and the reaction was carried out from 400 nM (another three-fold dilution when dispensed). The human serum was diluted and dispensed in the culture medium to a final concentration of 25%, and the mixture of the cells, antibodies, and human serum was incubated for 5 hours in a humidified incubator at 37° C. and 5% (v/v) C02 conditions.

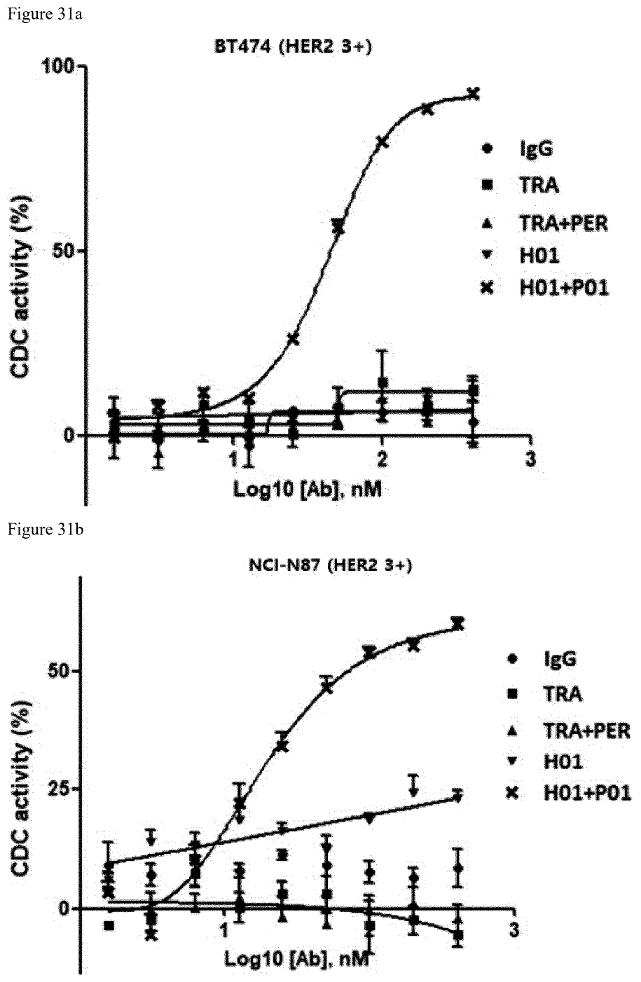

Cell Titer Glo-Reagent (Promega, G9243) previously dissolved at 4° C. was dispensed into each well in an equal volume of the mixed culture medium, and then the cell lysis was induced using a plate shaker (Allsheng, MX100-4A) with agitation at 500 rpm for 2 minutes. In order to stabilize the luminescence signal, the mixture was incubated at room temperature for 10 minutes and then analyzed using a plate reader equipment (Envision; PerkinElmer, 2105-0010) (FIGS. 31a to 31b). The complement-dependent cytotoxicity activity (CDC activity) was calculated as follows.

$$C D C activity ( \% ) = 100 \times [ 1 - ( luminescence with experimental antibody / luminiscence without antibody ) ] \qquad 〈 Equation 1 〉$$

Human IgG1, trastuzumab (TRA), trastuzumab+pertuzumab (TRA+PER), and H01 did not induce CDC responses in BT474 and NCI-N87 cell lines (FIGS. 31a to 31b). It was shown that CDC is induced in both cell lines only when H01 is treated in combination with P01 (FIGS. 31a to 31b).

### Example 14. Analysis of Antibody-Dependent Cytotoxicity

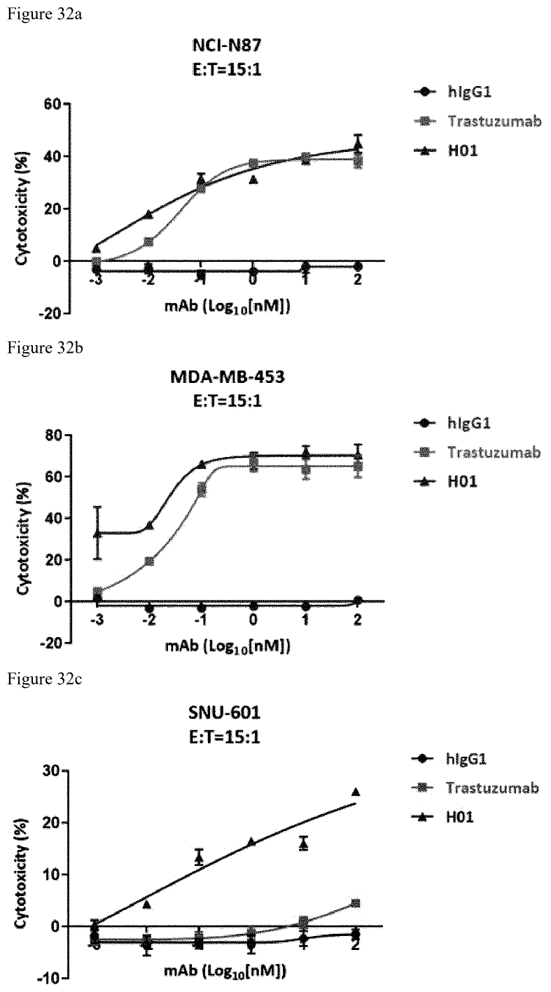

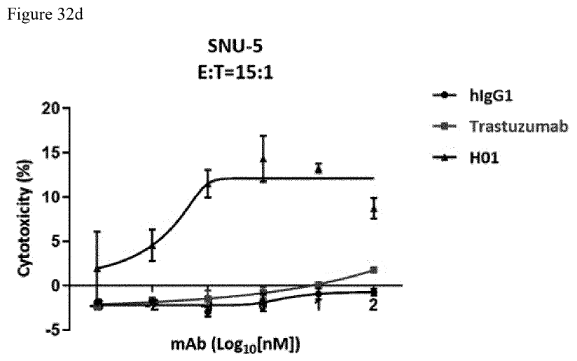

NCI-N87 (HER2 3+; high), MDA-MB-453(HER2 2+; Mid), SNU-601 (HER2 1+; low), and SNU-5 (HER2 1+; low) cancer cell lines were used for antibody-dependent cell-mediated cytotoxicity analysis (FIGS. 32a, 32b, 32c, and 32d). Each cancer cell line was seeded at 1.0×10⁴ cells/well in a 96-well plate. Thereafter, each antibody was diluted and treated in culture medium to an appropriate concentration. Peripheral blood mononuclear cells (PBMC) isolated on the same day were used as effector cells and treated at 1.5×10⁵ cells/well to make the number of PBMC 15 times more than the number of target cells (E:T ratio=15:1).

After treatment, the cells were incubated for 18 hours in a humidified incubator at 37° C. and 5% (v/v) C02 conditions, and then cytotoxicity was measured using a cytotoxicity detection kit (LDH) (Roche, 11644793001) (FIGS. 32a, 32b, 32c, and 32d). Cytotoxicity was calculated using the following formula for antibody-dependent cytotoxicity.

Cytotoxicity (%)=[(Test release-spontaneous release)/(Maximum release-spontaneous release)]×100 <Equation 2>

H01 showed excellent cytotoxicity at a low concentration compared to trastuzumab in NCI-N87 (HER2 3+; high) and MDA-MB-453 (HER2 2+; Mid) cancer cell lines (FIGS. 32a and 32b). Cytotoxicity analysis in SNU-601 (HER2 1+; low) and SNU-5 (HER2 1+; low) cancer cell lines H01 showed excellent cytotoxicity of H01 compared to that of trastuzumab (FIGS. 32c and 32d).

### Example 15. Evaluation of Efficacy in Xenograft Mouse Models

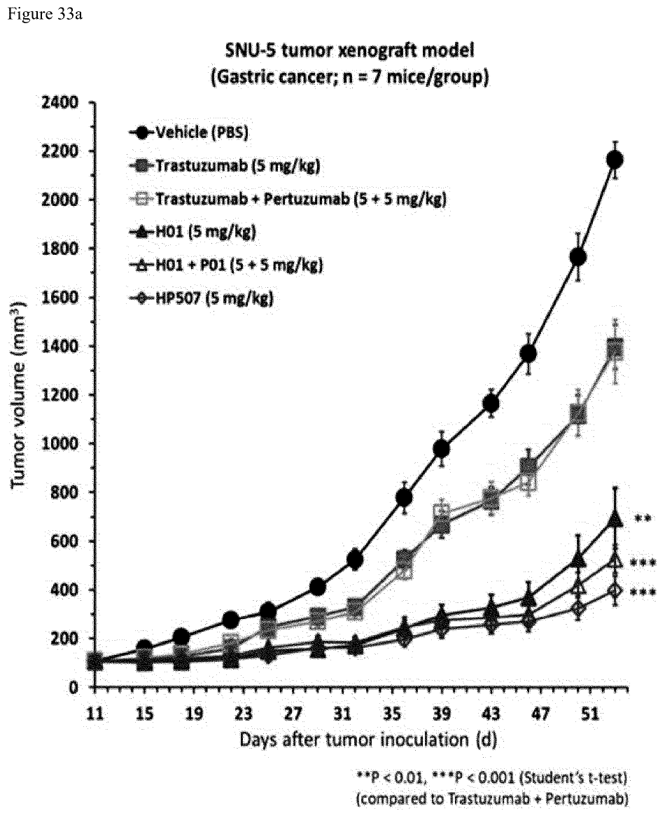

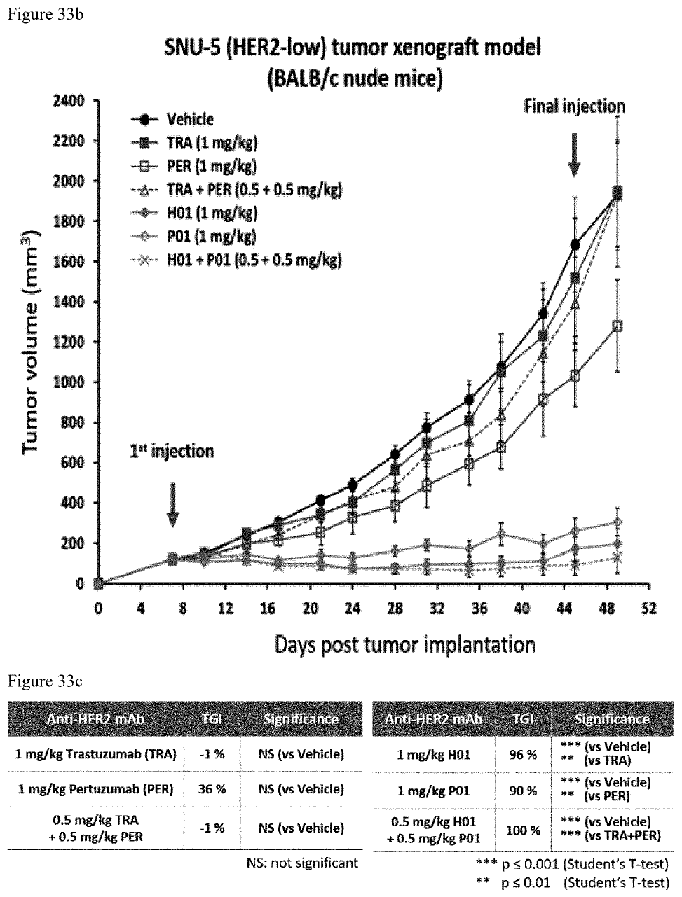

Efficacy in the SNU-5 (HER2 1+; low) gastric cancer cell line-derived xenograft model was evaluated using 6-week-old female SCID mice (C.B-17/NcrKoat-Prkdc^{scid}, Koatech) (FIG. 33a). The SNU-5 cancer cell line was diluted in PBS at 1×10⁷ cells/100 μL and mixed with MATRIGEL® Growth Factor Reduced (GFR) Basement Membrane Matrix (Corning, 354230) at a ratio of 1:1, and 100 μL of the mixture was transplanted subcutaneously into the right flank, and tumor growth was monitored. Mice were regrouped so that the average tumor volume was about 107 mm³, and PBS (vehicle), 5 mg/kg H01, 5 mg/kg H01+5 mg/kg P01, 5 mg/kg HP507, 5 mg/kg trastuzumab, 5 mg/kg trastuzumab+5 mg/kg pertuzumab were administered intravenously (I.V.) once a week for a total of 6 weeks (FIG. 33a). According to the analysis, H01 alone showed superior antitumor activity compared to trastuzumab and trastuzumab+pertuzumab (FIG. 33a). It was shown that H01+P01 induces improved antitumor activity compared to H01 alone, and HP507 induces the strongest antitumor activity in the SNU-5 gastric cancer xenograft model (FIG. 33a).

In addition, efficacy in the SNU-5 (HER2 1+; low) gastric cancer cell line-derived xenograft model was evaluated using 6-week-old female BALB/c-nu mice (ORIENT BIO INC.) (FIG. 33b). The SNU-5 cancer cell line was diluted in PBS at 1×10⁷ cells/100 μL and mixed with MATRIGEL® Growth Factor Reduced (GFR) Basement Membrane Matrix (Corning, 354230) at a ratio of 1:1, and 100 μL of the mixture was transplanted subcutaneously into the right flank, and tumor growth was observed. Mice were regrouped so that the average tumor volume was about 122 mm³, and 50 mg/kg Intravenous Immunoglobulin (IVIG; LIV-r, SK Plasma) was administered to all mice twice a week for 6 weeks (FIG. 33b). PBS (vehicle), 1 mg/kg trastuzumab, 1 mg/kg pertuzumab, 0.5 mg/kg trastuzumab+0.5 mg/kg pertuzumab, 1 mg/kg H01, 1 mg/kg P01, and 0.5 mg/kg H01+0.5 mg/kg P01 were administered intraperitoneally (I.P.) twice a week for a total of 6 weeks (FIG. 33b). The analysis showed that H01, P01, and H01+P01 induce superior antitumor activity compared to trastuzumab, pertuzumab, and trastuzumab+pertuzumab (FIG. 33b).

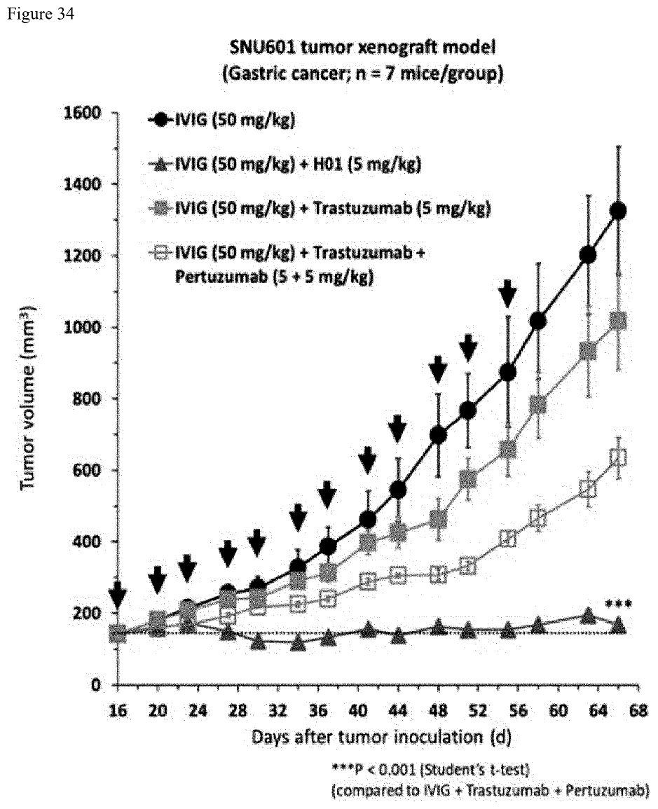

Efficacy in the SNU-601 (HER2 1+; low) gastric cancer cell line-derived xenograft model was evaluated using 6-week-old female SCID mice (C.B-17/NcrKoat-Prkdc^{scid}, Koatech). The SNU-5 cancer cell line was diluted in PBS at 1×10⁷ cells/100 μL and mixed with MATRIGEL® Growth Factor Reduced (GFR) Basement Membrane Matrix (Corning, 354230) at a ratio of 1:1, and 100 μL of the mixture was transplanted subcutaneously into the right flank, and tumor growth was observed. Mice were regrouped so that the average tumor volume was about 142 mm³, and PBS (vehicle), 5 mg/kg H01, 5 mg/kg trastuzumab, and 5 mg/kg trastuzumab+5 mg/kg pertuzumab were administered intraperitoneally (I.P.) twice a week for a total of 6 weeks (FIG. 34). Since there are no antibodies present in the blood of the SCID mice, 50 mg/kg Intravenous Immunoglobulin (IVIG; LIV-r, SK Plasma) was administered to all mice twice a week for 6 weeks to simulate the actual human blood environment (FIG. 34). In the SNU-601 gastric cancer xenograft model, H01 alone induced most superior antitumor activity (FIG. 34).

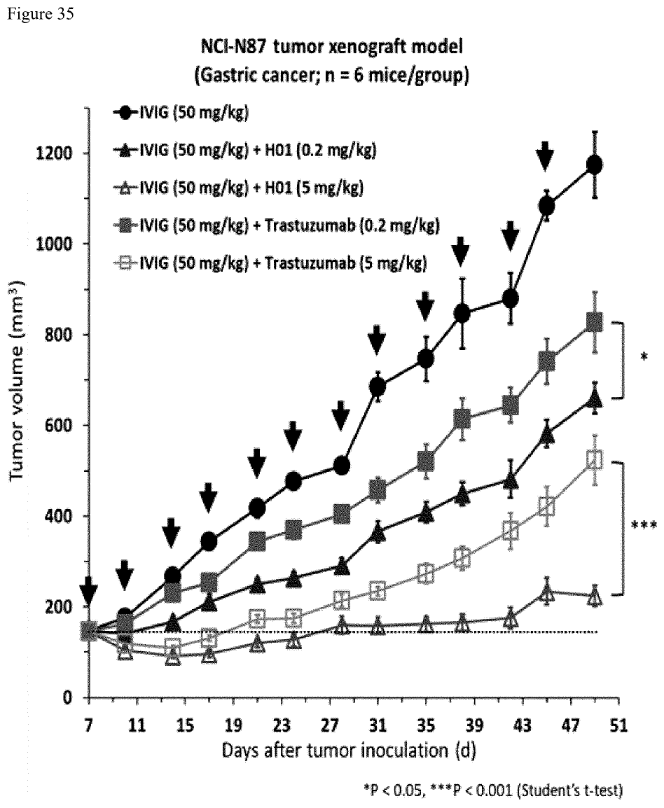

Efficacy in the NCI-N87 (HER2 3+; high) gastric cancer cell line-derived xenograft model was evaluated using 6-week-old female SCID mice (C.B-17/NcrKoat-Prkdc^{scid}, Koatech). The NCI-N87 cancer cell line was diluted in PBS at 5×10⁶ cells/100 μL and mixed with MATRIGEL® Growth Factor Reduced (GFR) Basement Membrane Matrix (Corning, 354230) at a ratio of 1:1, and 100 μL of the mixture was transplanted subcutaneously into the right flank, and tumor growth was observed. Mice were regrouped so that the average tumor volume was about 146 mm³, and PBS (vehicle), 0.2 mg/kg H01, 5 mg/kg H01, 0.2 mg/kg trastuzumab, and 5 mg/kg trastuzumab were administered intraperitoneally (I.P.) twice a week for a total of 6 weeks (FIG. 35). Since there are no antibodies present in the blood of the SCID mice, 50 mg/kg Intravenous Immunoglobulin (IVIG; LIV-r, SK Plasma) was administered to all mice twice a week for 6 weeks to simulate the actual human blood environment (FIG. 35). The analysis showed that H01 induces superior antitumor activity compared to trastuzumab when the antibodies were administered at 5 mg/kg and at 0.2 mg/kg (FIG. 35).

### Example 16. Evaluation of Antitumor Activity in CT26-HER2 Syngeneic Mouse Model

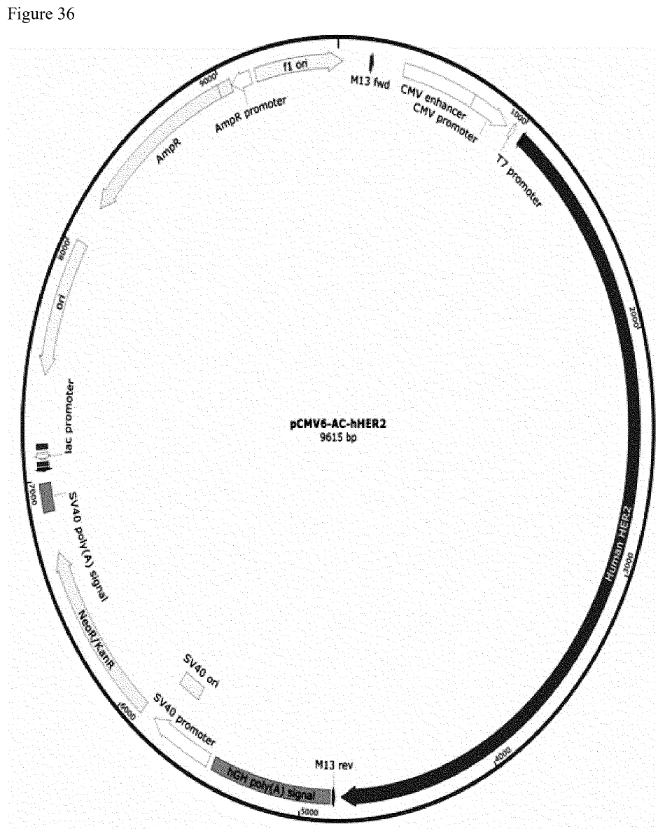

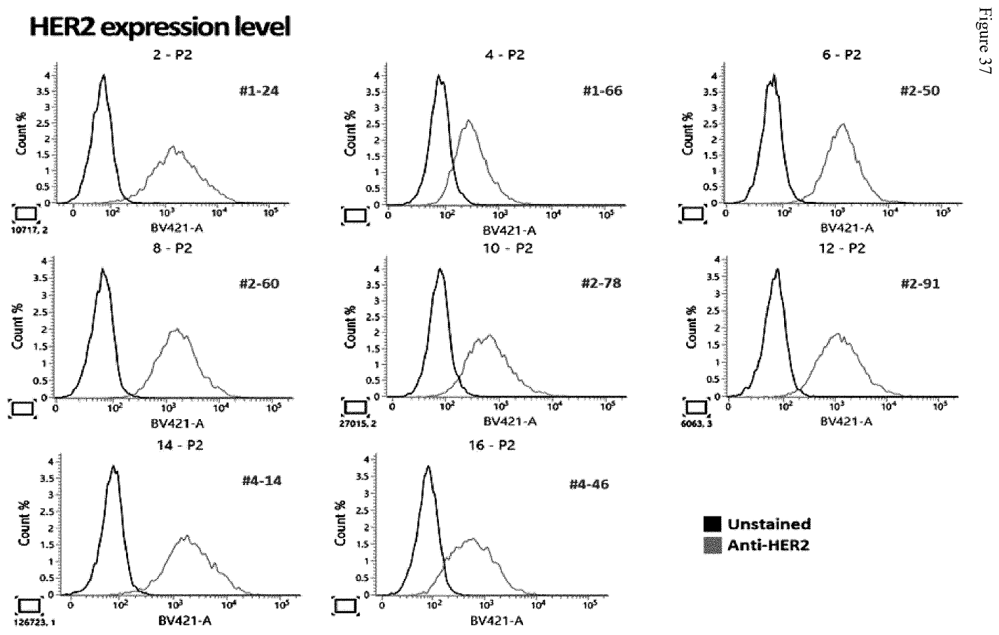

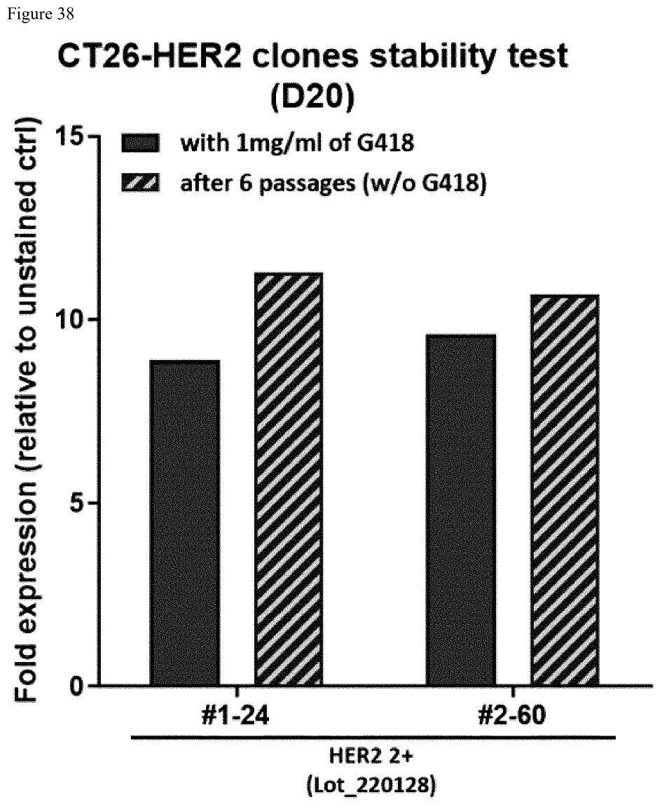

The nucleotide (SEQ ID NO: 566, Table 40) encoding the human HER2 protein (SEQ ID NO: 567, Table 40) was cloned into a protein expression vector (ORIGENE, PS100020) containing a neomycin-resistance gene to construct the human HER2 expression vector pCMV6-AC-hHER2 (FIG. 36, Table 40).

TABLE 40

| Name | Sequence | SEQ ID NO |
|---|---|---|
| Human HER2 | ATGGAGCTGGCGCCTTGTGCCGCTGGGGGCTCCTCCTCGCCCTCTTGCCCCCCGGAGC | 566 |
| nucleotide | CGCGAGCACCCAAGTGTGCACCGGCACAGACATGAAGCTGCGGCTCCCTGCCAGTCCCG | |
| sequence | AGACCCACCTGGACATGCTCCGCCACCTCTACCAGGGCTGCCAGGTGGTGCAGGGAAAC | |
| | CTGGAACTCACCTACCTGCCCACCAATGCCAGCCTGTCCTTCCTGCAGGATATCCAGGA | |
| | GGTGCAGGGCTACGTGCTCATCGCTCACAACCAAGTGAGGCAGGTCCCACTGCAGAGGC | |
| | TGCGGATTGTGCGAGGCACCCAGCTCTTTGAGGACAACTATGCCCTGGCCGTGCTAGAC | |
| | AATGGAGACCCGCTGAACAATACCACCCCTGTCACAGGGGCCTCCCCAGGAGGCCTGCG | |
| | GGAGCTGCAGCTTCGAAGCCTCACAGAGTCTTGAAAGGAGGGGTCTTGATCCAGCGGA | |
| | ACCCCCAGCTCTGCTACCAGGACACGATTTTGTGGAAGGACATCTTCCACAAGAACAAC | |
| | CAGCTGGCTCTCACACTGATAGACACCAACCGCTCTCGGGCCTGCCACCCCTGTTCTCC | |
| | GATGTGTAAGGGCTCCCGCTGCTGGGGAGAGAGTTCTGAGGATTGTCAGAGCCTGACGC | |
| | GCACTGTCTGTGCCGGTGGCTGTGCCCGCTGCAAGGGGCCAGCCCACTGACTGCTGC | |
| | CATGAGCAGTGTGCTGCCGGCTGCACGGGCCCCAAGCACTCTGACTGCCTGGCCTGCCT | |
| | CCACTTCAACCACAGTGGCATCTGTGAGCTGCACTGCCCAGCCCTGGTCACCTACAACA | |
| | CAGACACGTTTGAGTCCATGCCCAATCCCGAGGGCCGGTATACATTCGGCGCCAGCTGT | |
| | GTGACTGCCTGTCCCTACAACTACCTTTCTACGGACGTGGGATCCTGCACCCTCGTCTG | |
| | CCCCCTGCACAACCAAGAGGTGACAGCAGAGGATGGAACACAGCGGTGTGAGAAGTGCA | |
| | GCAAGCCCTGTGCCCGAGTGTGCTATGGTCTGGGCATGGAGCACTTGCGAGAGGTGAGG | |
| | GCAGTTACCAGTGCCAATATCCAGGAGTTTGCTGGCTGCAAGAAGATCTTTGGGAGCCT | |
| | GGCATTTCTGCCGGAGAGCTTTGATGGGGACCCAGCCTCCAACACTGCCCCGCTCCAGC | |
| | CAGAGCAGCTCCAAGTGTTTGAGACTCTGGAAGAGATCACAGGTTACCTATACATCTCA | |
| | GCATGGCCGGACAGCCTGCCTGACCTCAGCGTCTTCCAGAACCTGCAAGTAATCCGGGG | |
| | ACGAATTCTGCACAATGGCGCCTACTCGCTGACCCTGCAAGGGCTGGGCATCAGCTGGC | |
| | TGGGGCTGCGCTCACTGAGGGAACTGGGCAGTGGACTGGCCCTCATCCACCATAACACC | |
| | CACCTCTGCTTCGTGCACACGGTGCCCTGGGACCAGCTCTTTCGGAACCCGCACCAAGC | |
| | TCTGCTCCACACTGCCAACCGGCCAGAGGACGAGTGTGTGGGCGAGGGCCTGGCCTGCC | |
| | ACCAGCTGTGCGCCCGAGGGCACTGCTGGGGTCCAGGGCCCACCCAGTGTGTCAACTGC | |
| | AGCCAGTTCCTTCGGGGCCAGGAGTGCGTGGAGGAATGCCGAGTACTGCAGGGGCTCCC | |
| | CAGGGAGTATGTGAATGCCAGGCACTGTTTGCCGTGCCACCCTGAGTGTCAGCCCCAGA | |
| | ATGGCTCAGTGACCTGTTTTGGACCGGAGGCTGACCAGTGTGTGGCCTGTGCCCACTAT | |
| | AAGGACCCTCCCTTCTGCGTGGCCCGCTGCCCCAGCGGTGTGAAACCTGACCTCTCCTA | |
| | CATGCCCATCTGGAAGTTTCCAGATGAGGAGGGCGCATGCCAGCCTTGCCCCATCAACT | |
| | GCACCCACTCCTGTGTGGACCTGGATGACAAGGGCTGCCCCGCCGAGCAGAGAGCCAGC | |
| | CCTCTGACGTCCATCATCTCTGCGGTGGTTGGCATTCTGCTGGTCGTGGTCTTGGGGGT | |
| | GGTCTTTGGGATCCTCATCAAGCGACGGCAGCAGAAGATCCGGAAGTACACGATGCGGA | |
| | GACTGCTGCAGGAAACGGAGCTGGTGGAGCCGCTGACACCTAGCGGAGCGATGCCCAAC | |
| | CAGGCGCAGATGCGGATCCTGAAAGAGACGGAGCTGAGGAAGGTGAAGGTGCTTGGATC | |
| | TGGCGCTTTTGGCACAGTCTACAAGGGCATCTGGATCCCTGATGGGGAGAATGTGAAAA | |
| | TTCCAGTGGCCATCAAAGTGTTGAGGGAAAACACATCCCCCAAAGCCAACAAAGAAATC | |
| | TTAGACGAAGCATACGTGATGGCTGGTGTGGGCTCCCCATATGTCTCCCGCCTTCTGGG | |
| | CATCTGCCTGACATCCACGGTGCAGCTGGTGACACAGCTTATGCCCTATGGCTGCCTCT | |
| | TAGACCATGTCCGGGAAAACCGCGGACGCCTGGGCTCCCAGGACCTGCTGAACTGGTGT | |
| | ATGCAGATTGCCAAGGGGATGAGCTACCTGGAGGATGTGCGGCTCGTACACAGGGACTT | |
| | GGCCGCTCGGAACGTGCTGGTCAAGAGTCCCAACCATGTCAAAATTACAGACTTCGGGC | |
| | TGGCTCGGCTGCTGGACATTGACGAGACAGAGTACCATGCAGATGGGGGCAAGGTGCCC | |
| | ATCAAGTGGATGGCGCTGGAGTCCATTCTCCGCCGGCGGTTCACCCACCAGAGTGATGT | |
| | GTGGAGTTATGGTGTGACTGTGTGGGAGCTGATGACTTTTGGGGCCAAACCTTACGATG | |
| | GGATCCCAGCCCGGGAGATCCCTGACCTGCTGGAAAAGGGGGAGCGGCTGCCCCAGCCC | |
| | CCCATCTGCACCATTGATGTCTACATGATCATGGTCAAATGTTGGATGATTGACTCTGA | |
| | ATGTCGGCCAAGATTCCGGGAGTTGGTGTCTGAATTCTCCCGCATGGCCAGGGACCCCC | |
| | AGCGCTTTGTGGTCATCCAGAATGAGGACTTGGGCCCAGCCAGTCCCTTGGACAGCACC | |
| | TTCTACCGCTCACTGCTGGAGGACGATGACATGGGGGACCTGGTGGATGCTGAGGAGTA | |
| | TCTGGTACCCCAGCAGGGCTTCTTCTGTCCAGACCCTGCCCCGGGCGCTGGGGGCATGG | |
| | TCCACCACAGGCACCGCAGCTCATCTACCAGGAGTGGCGGTGGGGACCTGACACTAGGG | |
| | CTGGAGCCCTCTGAAGAGGAGGCCCCCAGGTCTCCACTGGCACCCTCCGAAGGGGCTGG | |
| | CTCCGATGTATTTGATGGTGACCTGGGAATGGGGGCAGCCAAGGGGCTGCAAAGCCTCC | |
| | CCACACATGACCCCAGCCCTCTACAGCGGTACAGTGAGGACCCCACAGTACCCCTGCCC | |
| | TCTGAGACTGATGGCTACGTTGCCCCCCTGACCTGCAGCCCCCAGCCTGAATATGTGAA | |
| | CCAGCCAGATGTTCGGCCCCAGCCCCCTTCGCCCCGAGAGGGCCCTCTGCCTGCTGCCC | |
| | GACCTGCTGGTGCCACTCTGGAAAGGCCCAAGACTCTCTCCCCAGGGAAGAATGGGGTC | |
| | GTCAAAGACGTTTTTGCCTTTGGGGGTGCCGTGGAGAACCCCGAGTACTTGACACCCCA | |
| | GGGAGGAGCTGCCCCTCAGCCCCACCCTCCTCCTGCCTTCAGCCCAGCCTTCGACAACC | |
| | TCTATTACTGGGACCAGGACCCACCAGAGCGGGGGGCTCCACCCAGCACCTTCAAAGGG | |
| | ACACCTACGGCAGAGAACCCAGAGTACCTGGGTCTGGACGTGCCAGTG | |
| Human HER2 | MELAALCRWGLLLALLPPGAASTQVCTGTDMKLRLPASPETHLDMLRHLYQGCQVVGN | 567 |
| protein | LELTYLPTNASLSFLQDIQEVQGYVLIAHNQVRQVPLQRLRIVRGTQLFEDNYALAVLD | |
| sequence | NGDPLNNTTPVTGASPGGLRELQLRSLTEILKGGVLIQRNPQLCYQDTILWKDIFHKNN | |
| | QLALTLIDTNRSRACHPCSPMCKGSRCWGESSEDCQSLTRTVCAGGCARCKGPLPTDCC | |
| | HEQCAAGCTGPKHSDCLACLHFNHSGICELHCPALVTYNTDTFESMPNPEGRYTFGASC | |
| | VTACPYNYLSTDVGSCTLVCPLHNQEVTAEDGTQRCEKCSKPCARVCYGLGMEHLREVR | |
| | AVTSANIQEFAGCKKIFGSLAFLPESFDGDPASNTAPLQPEQLQVFETLEEITGYLYIS | |
| | AWPDSLPDLSVFQNLQVIRGRILHNGAYSLTLQGLGISWLGLRSLRELGSGLALIHHNT | |
| | HLCFVHTVPWDQLFRNPHQALLHTANRPEDECVGEGLACHQLCARGHCWGPGPTQCVNC | |
| | SQFLRGQECVEECRVLQGLPREYVNARHCLPCHPECQPQNGSVTCFGPEADQCVACAHY | |
| | KDPPFCVARCPSGVKPDLSYMPIWKFPDEEGACQPCPINCTHSCVDLDDKGCPAEQRAS | |
| | PLTSIISAVVGILLVVVLGVVFGILIKRRQQKIRKYTMRRLLQETELVEPLTPSGAMPN | |
| | QAQMRILKETELRKVKVLGSGAFGTVYKGIWIPDGENVKIPVAIKVLRENTSPKANKEI | |
| | LDEAYVMAGVGSPYYSRLLGICLTSTVQLVTQLMPYGCLLDHVRENRGRLGSQDLLNWC | |
| | MQIAKGMSYLEDVRLVHRDLAARNVLVKSPNHVKITDFGLARLLDIDETEYHADGGKVP | |
| | IKWMALESILRRRFTHQSDVWSYGVTVWELMTFGAKPYDGIPAREIPDLLEKGERLPQP | |
| | PICTIDVYMIMVKCWMIDSECRPRFRELVSEFSRMARDPQRFVVIQNEDLGPASPLDST | |
| | FYRSLLEDDDMGDLVDAEEYLVPQQGFFCPDPAPGAGGMVHHRHRSSSTRSGGGDLTLG | |
| | LEPSEEEAPRSPLAPSEGAGSDVFDGDLGMGAAKGLQSLPTHDPSPLQRYSEDPTVPLP | |
| | SETDGYVAPLTCSPQPEYVNQPDVRPQPPSPREGPLPAARPAGATLERPKTLSPGKNGV | |
| | VKDVFAFGGAVENPEYLTPQGGAAPQPHPPPAFSPAFDNLYYWDQDPPERGAPPSTFKG | |
| | TPTAENPEYLGLDVPV | |

The mouse large intestine-derived CT26 cancer cells were transfected with the pCMV6-AC-hHER2 human HER2 expression vector using the lipofectamine 2000 transfection reagent (Invitrogen, 11668-019). Only cells transfected with the pCMV6-AC-hHER2 human HER2 expression vector were selected by incubating the transfected cells in culture medium containing 1 mg/mL G418 (Invivogen, ant-gn-5) for 14 days. The top 3% clones in terms of HER2 expression was sorted into a 96-well plate (ThermoFisher, 167008) with 1 cell per well using SH800S Cell Sorter (SONY). Selection was performed by incubation in G418-containing medium for 21 days, and a total of 8 CT26 mouse large intestine cancer cell line clones expressing human HER2 were obtained, and human HER2 expression in these cells was monitored by flow cytometer (BD Biosciences, FACSverse) analysis after staining with the anti-human HER2-BV421 (BD, 744811) (FIG. 37, Table 41).

TABLE 41
ΔGMFI (of unstained control)

| | |
|---|---|
| #1-24 | 27.3 |
| #1-66 | 3.7 |
| #2-50 | 19.1 |
| #2-60 | 25.9 |
| #2-78 | 7.5 |
| #2-91 | 17.5 |
| #4-14 | 26.8 |
| #4-46 | 6.5 |

Alter subculturing six times tor 20 days in a (G418-free environment, the cells were stained with the anti-human HER2-BV421 (BD, 744811), and the level of human HER2 expression was measured. It was shown that the level of human HER2 expression was not reduced in cells grown without G418 compared to cells grown with G418 (FIG. 38, Table 42).

TABLE 42
ΔGMFI (of unstained control)

| | |
|---|---|
| #1-24 | 27.3 |
| #1-66 | 3.7 |
| #2-50 | 19.1 |
| #2-60 | 25.9 |
| #2-78 | 7.5 |
| #2-91 | 17.5 |
| #4-14 | 26.8 |
| #4-46 | 6.5 |

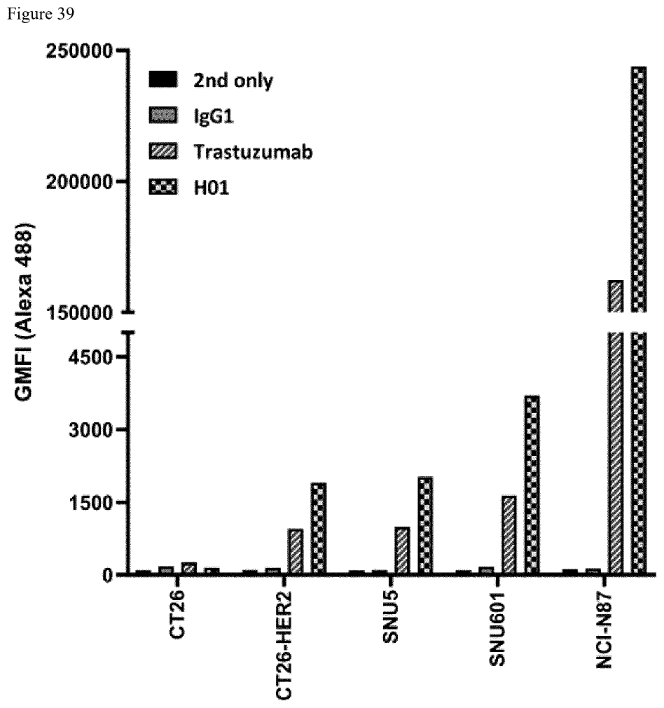

To compare the cell surface Fc loads among the parental CT26, CT26-HER2 cell line (Clone #2-60) and human cancer cell lines (SNU5, SNU601, and NCI-N87), each cell was allowed to bind to 100 nM human IgG1 (Bio X cell, BE0297), 100 nM trastuzumab (TRA), and 100 nM H01 antibody at 4° C. for 30 minutes in a 96-well v-bottom plate (Corning, 3363). Thereafter, they were treated with the Alexa 488 fluorescence-conjugated anti-human IgG Fcγ Fab antibody (Jackson ImmunoResearch, 109-547-008), and the Fc loads on the cells were quantified using a flow cytometer (FIG. 39, Table 43). CT26-HER2 cell line (Clone #2-60) was shown to express human HER2 at a level similar to that of SNU5 (FIG. 39, Table 43). In addition, it was shown that in the CT26-HER2 cell line, treatment of 100 nM H01 results in increased Fc loads on the cell surface compared to that of 100 nM trastuzumab (TRA).

TABLE 43

| | | CT26-HER2 | | | |
|---|---|---|---|---|---|
| GMFI | CT26 | (#2-60) | SNU5 | SNU601 | NCI-N87 |
| 2nd only | 99 | 102 | 94 | 97 | 116 |
| 100 nM IgG1 | 176 | 150 | 101 | 173 | 132 |
| isotype | | | | | |
| 100 nM | 252 | 959 | 991 | 1642 | 162433 |
| Trastuzumab | | | | | |
| 100 nM H01 | 147 | 1907 | 2028 | 3703 | 243897 |

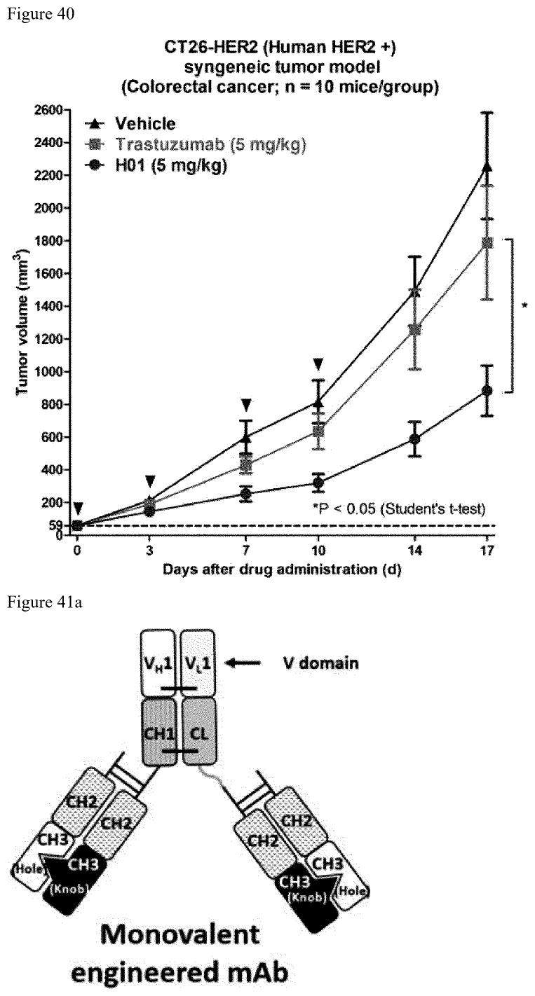

Efficacy in the CT26-HER2 (Clone #2-60) syngeneic mouse model was evaluated using 6-week-old female Balb/c mice (ORIENT BIO INC.). PBS (vehicle), 5 mg/kg trastuzumab, and 5 mg/kg H01 were administered intraperitoneally (I.P.) twice a week for a total of 2 weeks (FIG. 40). The analysis showed that H01 induces superior antitumor activity compared to trastuzumab (FIG. 40).

### Example 17. Design, Preparation and Analysis of Novel Antibody Targeting Glypican-3 (GPC3)

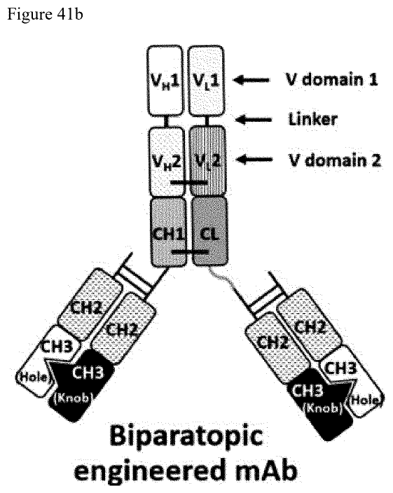

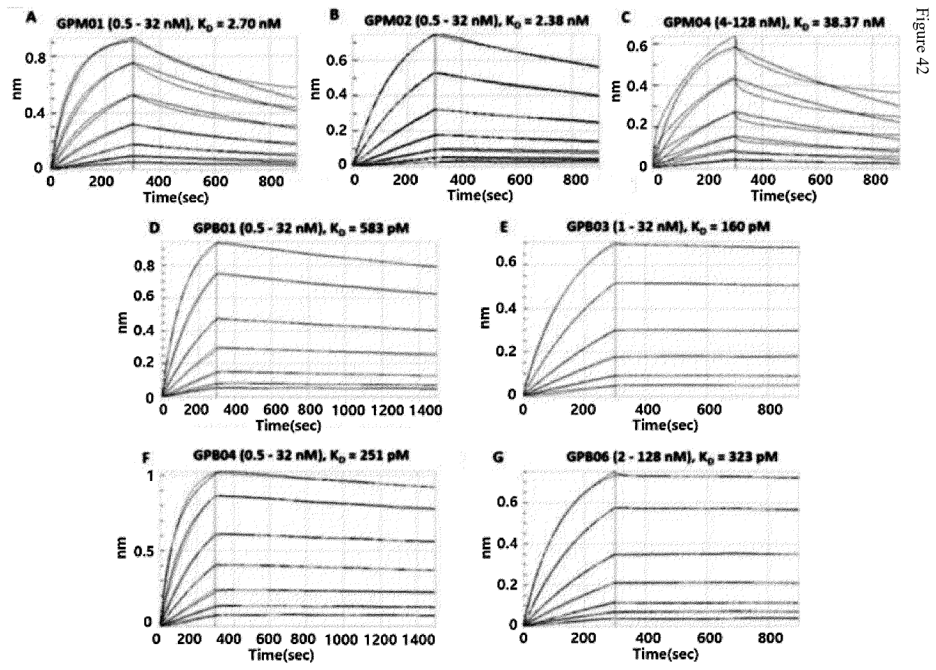

The variant light chain and heavy chain polypeptide sequences of the antibodies that specifically recognize the glypican-3 (GPC-3) protein are shown in Table 44. For GPM01, expression vectors consisting of the sequences corresponding to Fc-Hole (SEQ ID NO: 7), GPM01 HC (SEQ ID NO: 67), and GPM01 LC (SEQ ID NO: 68) were co-transfected into EXPICHO-S™ (Gibco, A29127) (FIG. 41a, Table 44), and purification and analysis were performed in the same manner as described in Example 1. Expression, purification and analysis were performed for GPM02, GPM04, GPB01, GPB03, GPB04, and GPB06 in the same manner as mentioned above (FIGS. 41a to 41b, Table 44). GPM01, GPM02, and GPM04 bind monovalently to different epitopes of the antigen and have structures consisting of two Fc domains (FIG. 41a). GPB01, GPB03, GPB04, and GPB06 have structures in which the variable regions of GPM01, GPM02, and GPM04 are linked with a polypeptide linker (SEQ ID NO: 48, SEQ ID NO: 50), and bind biparatopically to GPC-3, and have two Fc domains (FIG. 41b).

TABLE 44

| Name | Sequence | SEQ ID NO |
|---|---|---|
| GPM01 HC | EVQLVESGGGLVKPGGSLKLSCAASGFTFSRYAMSWVRQIPEKCLE | 67 |
| | WVAAIDSSGGDTYYLDTVKDRFTISRDNANNTLHLQMRSLRSEDTA | |
| | LYYCVRQGGAYWGQGTLVTVSSASTKGPSVFPLAPSSKSTSGGTAAL | |
| | GCLVKDYFPEPVTVSWNSGALTSGVHTFPAVLQSSGLYSLSSVVTVP | |
| | SSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| GPM01 LC | DVVMTQTPLTLSVTIGQPASISCKSSQSLLDSDGKTYLNWLLQRPGQS | 68 |
| | PKRLIYLVSKLDSGAPDRFTGSGSGTDFTLKISRVEAEDLGIYYCWQG | |
| | THFPLTFGCGTKLEIKRTVAAPSVFIFPPSDEQLKSGTASVVCLLNNFY | |
| | PREAKVQWKVDNALQSGNSQESVTEQDSKDSTYSLSSTLTLSKADY | |
| | EKHKVYACEVTHQGLSSPVTKSFNRGECGGGGSGGGGSGGGGSEPK | |
| | SSDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQD | |
| | WLNGKEYKCKVSNKALPAPIEKTISKAKGQPREPQVYTLPPCREEMT | |
| | KNQVSLWCLVKGFYPSDIAVEWESNGQPENNYKTTPPVLDSDGSFFL | |
| | YSKLTVDKSRWQQGNVFSCSVMHEALHNHYTQKSLSLSPGK | |
| GPM02 HC | QVQLVQSGAEVKKPGASVKVSCKASGYTFTDYEMHWVRQAPGQCL | 69 |
| | EWMGALDPKTGDTAYSQKFKGRVTLTADKSTSTAYMELSSLTSEDT | |
| | AVYYCTRFYSYTYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| GPM02 LC | DVVMTQSPLSLPVTPGEPASISCRSSQSLVHSNRNTYLHWYLQKPGQ | 70 |
| | SPQLLIYKVSNRFSGVPDRFSGSGSGTDFTLKISRVEAEDVGVYYCSQ | |
| | NTHVPPTFGCGTKLEIKRTVAAPSVFIFPPSDEQLKSGTASVVCLLNNF | |
| | YPREAKVQWKVDNALQSGNSQESVTEQDSKDSTYSLSSTLTLSKAD | |
| | YEKHKVYACEVTHQGLSSPVTKSFNRGECGGGGSGGGGSGGGGSEP | |
| | KSSDKTHTCPPCPAPELLGGPSVFLFPPKPKDTLMISRTPEVTCVVVD | |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPCREE | |
| | MTKNQVSLWCLVKGFYPSDIAVEWESNGQPENNYKTTPPVLDSDGS | |
| | FFLYSKLTVDKSRWQQGNVFSCSVMHEALHNHYTQKSLSLSPGK | |
| GPM04 HC | EVQLVETGGGLVQPKGSLKLSCAASGFTFNASAMNWVRQAPGKCLE | 71 |
| | WVARIRSKSNNYAIYYADSVKDRFTISRDDSQSMLYLQMNNLKTED | |
| | TAMYYCVRDPGYYGNPWFAYWGQGTLVTVSSASTKGPSVFPLAPSS | |
| | KSTSGGTAALGCLVKDYFPEPVTVSWNSGALTSGVHTFPAVLQSSGL | |
| | YSLSSVVTVPSSSLGTQTYICNVNHKPSNTKVDKKVEPKSCDKTHTC | |
| | PPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLW | |
| | CLVKGFYPSDIAVEWESNGQPENNYKTTPPVLDSDGSFFLYSKLTVD | |
| | KSRWQQGNVFSCSVMHEALHNHYTQKSLSLSPGK | |
| GPM04 LC | QIVLTQSPAIMSAFPGEKVTMTCSASSSVSYMYWYQQKSGSSPRLLIY | 72 |
| | DTSNLASGVPVRFSGSGSGTSYSLTISRMEAEDAATYYCQQWSSYPL | |
| | TFGCGTKLEIKRTVAAPSVFIFPPSDEQLKSGTASVVCLLNNFYPREA | |
| | KVQWKVDNALQSGNSQESVTEQDSKDSTYSLSSTLTLSKADYEKHK | |
| | VYACEVTHQGLSSPVTKSFNRGECGGGGSGGGGSGGGGSEPKSSDK | |
| | THTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLN | |
| | GKEYKCKVSNKALPAPIEKTISKAKGQPREPQVYTLPPCREEMTKNQ | |
| | VSLWCLVKGFYPSDIAVEWESNGQPENNYKTTPPVLDSDGSFFLYSK | |
| | LTVDKSRWQQGNVFSCSVMHEALHNHYTQKSLSLSPGK | |
| GPB01 HC | QVQLVQSGAEVKKPGASVKVSCKASGYTFTDYEMHWVRQAPGQGL | 73 |
| | EWMGALDPKTGDTAYSQKFKGRVTLTADKSTSTAYMELSSLTSEDT | |
| | AVYYCTRFYSYTYWGQGTLVTVSSASTKGPSVFPLAPEVQLVESGG | |
| | GLVKPGGSLKLSCAASGFTFSRYAMSWVRQIPEKCLEWVAAIDSSGG | |
| | DTYYLDTVKDRFTISRDNANNTLHLQMRSLRSEDTALYYCVRQGGA | |
| | YWGQGTLVTVSSASTKGPSVFPLAPSSKSTSGGTAALGCLVKDYFPE | |
| | PVTVSWNSGALTSGVHTFPAVLQSSGLYSLSSVVTVPSSSLGTQTYIC | |
| | NVNHKPSNTKVDKKVEPKSCDKTHTCPPCPAPELLGGPSVFLFPPKP | |
| | KDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEA | |
| | LHNHYTQKSLSLSPGK | |
| GPB01 LC | DVVMTQSPLSLPVTPGEPASISCRSSQSLVHSNRNTYLHWYLQKPGQ | 74 |
| | SPQLLIYKVSNRFSGVPDRFSGSGSGTDFTLKISRVEAEDVGVYYCSQ | |
| | NTHVPPTFGQGTKLEIKRTVAAPSVFIFPPDVVMTQTPLTLSVTIGQPA | |
| | SISCKSSQSLLDSDGKTYLNWLLQRPGQSPKRLIYLVSKLDSGAPDRF | |
| | TGSGSGTDFTLKISRVEAEDLGIYYCWQGTHFPLTFGCGTKLEIKRTV | |
| | AAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSP | |
| | VTKSFNRGECGGGGSGGGGSGGGGSEPKSSDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALP | |
| | APIEKTISKAKGQPREPQVYTLPPCREEMTKNQVSLWCLVKGFYPSDI | |
| | AVEWESNGQPENNYKTTPPVLDSDGSFFLYSKLTVDKSRWQQGNVF | |
| | SCSVMHEALHNHYTQKSLSLSPGK | |
| GPB03 HC | QVQLVQSGAEVKKPGASVKVSCKASGYTFTDYEMHWVRQAPGQGL | 75 |
| | EWMGALDPKTGDTAYSQKFKGRVTLTADKSTSTAYMELSSLTSEDT | |
| | AVYYCTRFYSYTYWGQGTLVTVSSASTKGPSVFPLAPEVQLVETGG | |
| | GLVQPKGSLKLSCAASGFTFNASAMNWVRQAPGKCLEWVARIRSKS | |
| | NNYAIYYADSVKDRFTISRDDSQSMLYLQMNNLKTEDTAMYYCVR | |
| | DPGYYGNPWFAYWGQGTLVTVSSASTKGPSVFPLAPSSKSTSGGTA | |
| | ALGCLVKDYFPEPVTVSWNSGALTSGVHTFPAVLQSSGLYSLSSVVT | |
| | VPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| GPB03 LC | DVVMTQSPLSLPVTPGEPASISCRSSQSLVHSNRNTYLHWYLQKPGQ | 76 |
| | SPQLLIYKVSNRFSGVPDRFSGSGSGTDFTLKISRVEAEDVGVYYCSQ | |
| | NTHVPPTFGQGTKLEIKRTVAAPSVFIFPPQIVLTQSPAIMSAFPGEKV | |
| | TMTCSASSSVSYMYWYQQKSGSSPRLLIYDTSNLASGVPVRFSGSGS | |
| | GTSYSLTISRMEAEDAATYYCQQWSSYPLTFGCGTKLEIKRTVAAPS | |
| | VFIFPPSDEQLKSGTASVVCLLNNFYPREAKVQWKVDNALQSGNSQE | |
| | SVTEQDSKDSTYSLSSTLTLSKADYEKHKVYACEVTHQGLSSPVTKS | |
| | FNRGECGGGGSGGGGSGGGGSEPKSSDKTHTCPPCPAPELLGGPSVF | |
| | LFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIE | |
| | KTISKAKGQPREPQVYTLPPCREEMTKNQVSLWCLVKGFYPSDIAVE | |
| | WESNGQPENNYKTTPPVLDSDGSFFLYSKLTVDKSRWQQGNVFSCS | |
| | VMHEALHNHYTQKSLSLSPGK | |
| GPB04 HC | EVQLVESGGGLVKPGGSLKLSCAASGFTFSRYAMSWVRQIPEKILEW | 77 |
| | VAAIDSSGGDTYYLDTVKDRFTISRDNANNTLHLQMRSLRSEDTALY | |
| | YCVRQGGAYWGQGTLVTVSSASTKGPSVFPLAPQVQLVQSGAEVKK | |
| | PGASVKVSCKASGYTFTDYEMHWVRQAPGQCLEWMGALDPKTGDT | |
| | AYSQKFKGRVTLTADKSTSTAYMELSSLTSEDTAVYYCTRFYSYTY | |
| | WGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPK | |
| | DTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAK | |
| | GQPREPQVYTLPPCREEMTKNQVSLWCLVKGFYPSDIAVEWESNGQ | |
| | PENNYKTTPPVLDSDGSFFLYSKLTVDKSRWQQGNVFSCSVMHEAL | |
| | HNHYTQKSLSLSPGK | |
| GPB04 LC | DVVMTQTPLTLSVTIGQPASISCKSSQSLLDSDGKTYLNWLLQRPGQS | 78 |
| | PKRLIYLVSKLDSGAPDRFTGSGSGTDFTLKISRVEAEDLGIYYCWQG | |
| | THFPLTFGAGTKLEIKRTVAAPSVFIFPPDVVMTQSPLSLPVTPGEPASI | |
| | SCRSSQSLVHSNRNTYLHWYLQKPGQSPQLLIYKVSNRFSGVPDRFS | |
| | GSGSGTDFTLKISRVEAEDVGVYYCSQNTHVPPTFGCGTKLEIKRTV | |
| | AAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSP | |
| | VTKSFNRGECGGGGSGGGGSGGGGSEPKSSDKTHTCPPCPAPELLGG | |
| | PSVFLFPPKPKDTLMISRTPEVTCVVVDVSHEDPEVKFNWYVDGVEV | |
| | HNAKTKPREEQYNSTYRVVSVLTVLHQDWLNGKEYKCKVSNKALP | |
| | APIEKTISKAKGQPREPQVYTLPPCREEMTKNQVSLWCLVKGFYPSDI | |
| | AVEWESNGQPENNYKTTPPVLDSDGSFFLYSKLTVDKSRWQQGNVF | |
| | SCSVMHEALHNHYTQKSLSLSPGK | |
| GPB06 HC | EVQLVETGGGLVQPKGSLKLSCAASGFTFNASAMNWVRQAPGKGLE | 79 |
| | WVARIRSKSNNYAIYYADSVKDRFTISRDDSQSMLYLQMNNLKTED | |
| | TAMYYCVRDPGYYGNPWFAYWGQGTLVTVSSASTKGPSVFPLAPQ | |
| | VQLVQSGAEVKKPGASVKVSCKASGYTFTDYEMHWVRQAPGQCLE | |
| | WMGALDPKTGDTAYSQKFKGRVTLTADKSTSTAYMELSSLTSEDTA | |
| | VYYCTRFYSYTYWGQGTLVTVSSASTKGPSVFPLAPSSKSTSGGTAA | |
| | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPELLG | |
| | GPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPS | |
| | DIAVEWESNGQPENNYKTTPPVLDSDGSFFLYSKLTVDKSRWQQGN | |
| | VFSCSVMHEALHNHYTQKSLSLSPGK | |
| GPB06 LC | QIVLTQSPAIMSAFPGEKVTMTCSASSSVSYMYWYQQKSGSSPRLLIY | 80 |
| | DTSNLASGVPVRFSGSGSGTSYSLTISRMEAEDAATYYCQQWSSYPL | |
| | TFGGGTKLEIKRTVAAPSVFIFPPDVVMTQSPLSLPVTPGEPASISCRSS | |
| | QSLVHSNRNTYLHWYLQKPGQSPQLLIYKVSNRFSGVPDRFSGSGSG | |
| | TDFTLKISRVEAEDVGVYYCSQNTHVPPTFGCGTKLEIKRTVAAPSVF | |
| | IFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSFN | |
| | RGECGGGGSGGGGSGGGGSEPKSSDKTHTCPPCPAPELLGGPSVFLFP | |
| | PKPKDTLMISRTPEVTCVVVDVSHEDPEVKFNWYVDGVEVHNAKTK | |
| | PREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWES | |
| | NGQPENNYKTTPPVLDSDGSFFLYSKLTVDKSRWQQGNVFSCSVMH | |
| | EALHNHYTQKSLSLSPGK | |

TABLE 45

| Name | Sequence | SEQ ID NO |
|---|---|---|
| GPM01 HC | GAAGTTCAGCTTGTCGAAAGTGGCGGAGGTCTTGTAAAGCCAGGA | 504 |
| | GGGTCCCTGAAGTTGAGTTGTGCTGCCTCTGGCTTCACCTTTTCAC | |
| | GGTATGCCATGTCCTGGGTTCGACAGATACCTGAGAAGTGTCTTG | |
| | AATGGGTGGCCGCAATAGACAGTAGCGGTGGTGACACCTATTACC | |
| | TCGACACAGTCAAAGACCGCTTTACTATCTCACGCGATAACGCCA | |
| | ACAATACCCTGCACTTGCAGATGCGATCACTTCGTTCAGAAGACA | |
| | CTGCTCTTTACTATTGTGTACGCCAAGGGGGAGCATACTGGGGTC | |
| | AGGGAACACTGGTTACCGTGTCTTCAGCTAGCACCAAAGGACCTA | |
| | GTGTTTTTCCTCTTGCCCCTTCCTCAAAGTCTACCTCTGGGGGGAC | |
| | AGCCGCTCTGGGCTGCCTGGTCAAGGATTATTTCCCAGAGCCTGT | |
| | CACTGTCAGTTGGAACTCTGGAGCCTTGACTTCTGGTGTTCATACA | |
| | TTTCCTGCTGTCCTTCAAAGCAGCGGCTTGTACTCATTGTCTTCTGT | |
| | TGTGACAGTACCCTCAAGCAGCCTCGGCACTCAGACATACATCTG | |
| | CAATGTCAACCACAAACCCTCAAATACAAAGGTAGATAAAAAAG | |
| | TCGAACCAAAGTCTTGTGACAAAACTCACACGTGCCCACCGTGCC | |
| | CAGCACCTGAACTCCTGGGGGGACCGTCAGTCTTCCTCTTCCCCCC | |
| | AAAACCCAAGGACACCCTCATGATCTCCCGGACCCCTGAGGTCAC | |
| | ATGCGTGGTGGTGGACGTGAGCCACGAAGACCCTGAGGTCAAGTT | |
| | CAACTGGTACGTGGACGGCGTGGAGGTGCATAATGCCAAGACAA | |
| | AGCCGCGGGAGGAGCAGTACAACAGCACGTACCGTGTGGTCAGC | |
| | GTCCTCACCGTCCTGCACCAGGACTGGCTGAATGGCAAGGAGTAC | |
| | AAGTGCAAGGTCTCCAACAAAGCCCTCCCAGCCCCCATCGAGAAA | |
| | ACCATCTCCAAAGCCAAAGGGCAGCCCCGAGAACCACAGGTGTA | |
| | CACCCTGCCCCCATGCCGGGAGGAGATGACCAAGAACCAGGTCA | |
| | GCCTGTGGTGCCTGGTCAAAGGCTTCTATCCCAGCGACATCGCCG | |
| | TGGAGTGGGAGAGCAATGGGCAGCCGGAGAACAACTACAAGACC | |
| | ACGCCTCCCGTGCTGGACTCCGACGGCTCCTTCTTCCTCTACAGCA | |
| | AGCTCACCGTGGACAAGAGCAGGTGGCAGCAGGGGAACGTCTTC | |
| | TCATGCTCCGTGATGCATGAGGCTCTGCACAACCACTACACGCAG | |
| | AAGAGCCTCTCCCTGTCTCCGGGTAAA | |
| GPM01 LC | GACGTGGTTATGACTCAGACTCCTCTGACTCTCTCCGTCACTATTG | 505 |
| | GTCAGCCCGCTTCCATATCATGTAAATCATCACAATCTCTTCTTGA | |
| | TAGCGATGGCAAGACTTATTTGAACTGGTTGTTGCAACGCCCAGG | |
| | TCAGAGCCCTAAGAGACTTATCTATTTGGTGAGCAAACTCGACAG | |
| | CGGTGCACCCGATCGTTTTACCGGAAGCGGCAGCGGCACCGATTT | |
| | CACACTGAAGATCAGTAGGGTCGAAGCTGAAGACCTGGGAATCT | |
| | ACTACTGCTGGCAAGGTACTCACTTTCCCCTGACTTTCGGCTGCGG | |
| | TACTAAACTTGAGATCAAACGTACGGTGGCAGCTCCCAGCGTTTT | |
| | TATCTTTCCCCCATCCGACGAGCAGCTCAAGAGTGGCACTGCCTCT | |
| | GTAGTTTGTTTGCTGAATAACTTCTATCCACGTGAAGCAAAAGTAC | |
| | AGTGGAAGGTCGATAATGCCCTTCAGAGCGGTAACAGCCAAGAA | |
| | AGTGTTACCGAGCAAGATTCCAAAGATTCCACTTACAGTCTGTCC | |
| | AGCACATTGACACTGAGTAAGGCTGATTACGAAAAACACAAGGT | |
| | GTACGCATGCGAGGTGACACACCAAGGTCTTTCATCTCCTGTAAC | |
| | TAAGAGCTTTAACCGGGGAGAATGTGGTGGTGGGGGCAGCGGGG | |
| | GCGGAGGTAGTGGAGGCGGCGGTAGTGAACCAAAGAGTAGTGAC | |
| | AAAACTCACACGTGCCCACCGTGCCCAGCACCTGAACTCCTGGGG | |
| | GGACCGTCAGTCTTCCTCTTCCCCCCAAAACCCAAGGACACCCTC | |
| | ATGATCTCCCGGACCCCTGAGGTCACATGCGTGGTGGTGGACGTG | |
| | AGCCACGAAGACCCTGAGGTCAAGTTCAACTGGTACGTGGACGGC | |
| | GTGGAGGTGCATAATGCCAAGACAAAGCCGCGGGAGGAGCAGTA | |
| | CAACAGCACGTACCGTGTGGTCAGCGTCCTCACCGTCCTGCACCA | |
| | GGACTGGCTGAATGGCAAGGAGTACAAGTGCAAGGTCTCCAACA | |
| | AAGCCCTCCCAGCCCCCATCGAGAAAACCATCTCCAAAGCCAAAG | |
| | GGCAGCCCCGAGAACCACAGGTGTACACCCTGCCCCCATGCCGGG | |
| | AGGAGATGACCAAGAACCAGGTCAGCCTGTGGTGCCTGGTCAAA | |
| | GGCTTCTATCCCAGCGACATCGCCGTGGAGTGGGAGAGCAATGGG | |
| | CAGCCGGAGAACAACTACAAGACCACGCCTCCCGTGCTGGACTCC | |
| | GACGGCTCCTTCTTCCTCTACAGCAAGCTCACCGTGGACAAGAGC | |
| | AGGTGGCAGCAGGGGAACGTCTTCTCATGCTCCGTGATGCATGAG | |
| | GCTCTGCACAACCACTACACGCAGAAGAGCCTCTCCCTGTCTCCG | |
| | GGTAAA | |
| GPM02 HC | CAGGTTCAGCTTGTGCAATCAGGCGCTGAAGTCAAAAAACCAGGA | 506 |
| | GCAAGCGTGAAAGTAAGCTGTAAGGCTTCCGGTTACACTTTCACC | |
| | GATTACGAAATGCACTGGGTACGCCAGGCTCCTGGACAATGTCTG | |
| | GAATGGATGGGCGCACTTGATCCAAAAACAGGTGACACTGCTTAC | |
| | AGTCAAAAATTCAAAGGTCGTGTCACTCTTACAGCAGATAAGTCC | |
| | ACCAGTACCGCTTATATGGAGCTTAGCTCCCTGACTTCCGAGGAC | |
| | ACCGCCGTGTATTATTGTACAAGATTCTACTCATATACTTACTGGG | |
| | GCCAAGGAACCCTGGTGACAGTGTCATCTGCTAGCACCAAAGGAC | |
| | CTAGTGTTTTTCCTCTTGCCCCTTCCTCAAAGTCTACCTCTGGGGG | |
| | GACAGCCGCTCTGGGCTGCCTGGTCAAGGATTATTTCCCAGAGCC | |
| | TGTCACTGTCAGTTGGAACTCTGGAGCCTTGACTTCTGGTGTTCAT | |
| | ACATTTCCTGCTGTCCTTCAAAGCAGCGGCTTGTACTCATTGTCTT | |
| | CTGTTGTGACAGTACCCTCAAGCAGCCTCGGCACTCAGACATACA | |
| | TCTGCAATGTCAACCACAAACCCTCAAATACAAAGGTAGATAAAA | |
| | AAGTCGAACCAAAGTCTTGTGACAAAACTCACACGTGCCCACCGT | |
| | GCCCAGCACCTGAACTCCTGGGGGGACCGTCAGTCTTCCTCTTCCC | |
| | CCCAAAACCCAAGGACACCCTCATGATCTCCCGGACCCCTGAGGT | |
| | CACATGCGTGGTGGTGGACGTGAGCCACGAAGACCCTGAGGTCA | |
| | AGTTCAACTGGTACGTGGACGGCGTGGAGGTGCATAATGCCAAGA | |
| | CAAAGCCGCGGGAGGAGCAGTACAACAGCACGTACCGTGTGGTC | |
| | AGCGTCCTCACCGTCCTGCACCAGGACTGGCTGAATGGCAAGGAG | |
| | TACAAGTGCAAGGTCTCCAACAAAGCCCTCCCAGCCCCCATCGAG | |
| | AAAACCATCTCCAAAGCCAAAGGGCAGCCCCGAGAACCACAGGT | |
| | GTACACCCTGCCCCCATGCCGGGAGGAGATGACCAAGAACCAGG | |
| | TCAGCCTGTGGTGCCTGGTCAAAGGCTTCTATCCCAGCGACATCG | |
| | CCGTGGAGTGGGAGAGCAATGGGCAGCCGGAGAACAACTACAAG | |
| | ACCACGCCTCCCGTGCTGGACTCCGACGGCTCCTTCTTCCTCTACA | |
| | GCAAGCTCACCGTGGACAAGAGCAGGTGGCAGCAGGGGAACGTC | |
| | TTCTCATGCTCCGTGATGCATGAGGCTCTGCACAACCACTACACG | |
| | CAGAAGAGCCTCTCCCTGTCTCCGGGTAAA | |
| GPM02 LC | GATGTTGTTATGACTCAGTCACCTCTCTCACTTCCTGTAACCCCTG | 507 |
| | GTGAACCAGCCTCTATAAGCTGCCGGTCAAGTCAAAGCCTGGTTC | |
| | ATAGCAACCGTAACACTTACCTTCACTGGTACTTGCAAAAACCTG | |
| | GTCAGTCCCCACAACTCTTGATCTACAAAGTCTCCAATCGCTTCTC | |
| | TGGAGTCCCTGACAGGTTTTCTGGTAGTGGATCAGGTACAGACTT | |
| | CACTTTGAAAATCAGCCGCGTTGAGGCCGAGGACGTGGGAGTGTA | |
| | TTATTGCTCTCAGAATACCCATGTACCCCCAACCTTTGGCTGTGGG | |
| | ACTAAACTCGAGATTAAACGTACGGTGGCAGCTCCCAGCGTTTTT | |
| | ATCTTTCCCCCATCCGACGAGCAGCTCAAGAGTGGCACTGCCTCT | |
| | GTAGTTTGTTTGCTGAATAACTTCTATCCACGTGAAGCAAAAGTAC | |
| | AGTGGAAGGTCGATAATGCCCTTCAGAGCGGTAACAGCCAAGAA | |
| | AGTGTTACCGAGCAAGATTCCAAAGATTCCACTTACAGTCTGTCC | |
| | AGCACATTGACACTGAGTAAGGCTGATTACGAAAAACACAAGGT | |
| | GTACGCATGCGAGGTGACACACCAAGGTCTTTCATCTCCTGTAAC | |
| | TAAGAGCTTTAACCGGGGAGAATGTGGTGGTGGGGGCAGCGGGG | |
| | GCGGAGGTAGTGGAGGCGGCGGTAGTGAACCAAAGAGTAGTGAC | |
| | AAAACTCACACGTGCCCACCGTGCCCAGCACCTGAACTCCTGGGG | |
| | GGACCGTCAGTCTTCCTCTTCCCCCCAAAACCCAAGGACACCCTC | |
| | ATGATCTCCCGGACCCCTGAGGTCACATGCGTGGTGGTGGACGTG | |
| | AGCCACGAAGACCCTGAGGTCAAGTTCAACTGGTACGTGGACGGC | |
| | GTGGAGGTGCATAATGCCAAGACAAAGCCGCGGGAGGAGCAGTA | |
| | CAACAGCACGTACCGTGTGGTCAGCGTCCTCACCGTCCTGCACCA | |
| | GGACTGGCTGAATGGCAAGGAGTACAAGTGCAAGGTCTCCAACA | |
| | AAGCCCTCCCAGCCCCCATCGAGAAAACCATCTCCAAAGCCAAAG | |
| | GGCAGCCCCGAGAACCACAGGTGTACACCCTGCCCCCATGCCGGG | |
| | AGGAGATGACCAAGAACCAGGTCAGCCTGTGGTGCCTGGTCAAA | |
| | GGCTTCTATCCCAGCGACATCGCCGTGGAGTGGGAGAGCAATGGG | |
| | CAGCCGGAGAACAACTACAAGACCACGCCTCCCGTGCTGGACTCC | |
| | GACGGCTCCTTCTTCCTCTACAGCAAGCTCACCGTGGACAAGAGC | |
| | AGGTGGCAGCAGGGGAACGTCTTCTCATGCTCCGTGATGCATGAG | |
| | GCTCTGCACAACCACTACACGCAGAAGAGCCTCTCCCTGTCTCCG | |
| | GGTAAA | |
| GPM04 HC | GAAGTGCAACTGGTCGAAACAGGCGGGGGACTGGTACAGCCCAA | 508 |
| | GGGATCTTTGAAACTTAGTTGTGCTGCTAGTGGGTTTACATTCAAT | |
| | GCCTCCGCAATGAACTGGGTAAGACAAGCTCCTGGCAAGTGCCTG | |
| | GAATGGGTGGCCCGTATTCGCTCTAAAAGTAATAACTACGCTATT | |
| | TATTACGCTGATTCTGTAAAGGATCGGTTTACAATAAGTCGGGAC | |
| | GACAGCCAATCCATGCTGTATCTCCAAATGAATAACCTGAAAACA | |
| | GAGGATACTGCCATGTACTATTGTGTGCGGGACCCAGGCTATTAC | |
| | GGGAATCCCTGGTTCGCCTATTGGGGACAGGGCACTCTGGTTACC | |
| | GTATCATCAGCTAGCACCAAAGGACCTAGTGTTTTTCCTCTTGCCC | |
| | CTTCCTCAAAGTCTACCTCTGGGGGGACAGCCGCTCTGGGCTGCC | |
| | TGGTCAAGGATTATTTCCCAGAGCCTGTCACTGTCAGTTGGAACTC | |
| | TGGAGCCTTGACTTCTGGTGTTCATACATTTCCTGCTGTCCTTCAA | |
| | AGCAGCGGCTTGTACTCATTGTCTTCTGTTGTGACAGTACCCTCAA | |
| | GCAGCCTCGGCACTCAGACATACATCTGCAATGTCAACCACAAAC | |
| | CCTCAAATACAAAGGTAGATAAAAAAGTCGAACCAAAGTCTTGTG | |
| | ACAAAACTCACACGTGCCCACCGTGCCCAGCACCTGAACTCCTGG | |
| | GGGGACCGTCAGTCTTCCTCTTCCCCCCAAAACCCAAGGACACCC | |
| | TCATGATCTCCCGGACCCCTGAGGTCACATGCGTGGTGGTGGACG | |
| | TGAGCCACGAAGACCCTGAGGTCAAGTTCAACTGGTACGTGGACG | |
| | GCGTGGAGGTGCATAATGCCAAGACAAAGCCGCGGGAGGAGCAG | |
| | TACAACAGCACGTACCGTGTGGTCAGCGTCCTCACCGTCCTGCAC | |
| | CAGGACTGGCTGAATGGCAAGGAGTACAAGTGCAAGGTCTCCAA | |
| | CAAAGCCCTCCCAGCCCCCATCGAGAAAACCATCTCCAAAGCCAA | |
| | AGGGCAGCCCCGAGAACCACAGGTGTACACCCTGCCCCCATGCCG | |
| | GGAGGAGATGACCAAGAACCAGGTCAGCCTGTGGTGCCTGGTCA | |
| | AAGGCTTCTATCCCAGCGACATCGCCGTGGAGTGGGAGAGCAATG | |
| | GGCAGCCGGAGAACAACTACAAGACCACGCCTCCCGTGCTGGACT | |
| | CCGACGGCTCCTTCTTCCTCTACAGCAAGCTCACCGTGGACAAGA | |
| | GCAGGTGGCAGCAGGGGAACGTCTTCTCATGCTCCGTGATGCATG | |
| | AGGCTCTGCACAACCACTACACGCAGAAGAGCCTCTCCCTGTCTC | |
| | CGGGTAAA | |
| GPM04 LC | CAAATTGTCCTCACCCAATCACCAGCTATAATGTCTGCTTTTCCCG | 509 |
| | GCGAGAAAGTAACCATGACTTGTAGCGCCTCTAGTAGCGTGTCAT | |
| | ATATGTATTGGTATCAACAAAAGAGCGGTAGTTCACCTCGACTCC | |
| | TTATCTACGACACAAGTAATCTCGCTAGTGGTGTCCCAGTCCGTTT | |
| | CTCCGGGAGCGGCAGCGGCACATCATACTCCCTGACCATCTCCAG | |
| | AATGGAGGCCGAGGACGCTGCCACATACTATTGTCAGCAGTGGAG | |
| | CTCATATCCTTTGACATTCGGTTGCGGTACTAAACTCGAAATCAAG | |
| | CGTACGGTGGCAGCTCCCAGCGTTTTTATCTTTCCCCCATCCGACG | |
| | AGCAGCTCAAGAGTGGCACTGCCTCTGTAGTTTGTTTGCTGAATA | |
| | ACTTCTATCCACGTGAAGCAAAAGTACAGTGGAAGGTCGATAATG | |
| | CCCTTCAGAGCGGTAACAGCCAAGAAAGTGTTACCGAGCAAGATT | |
| | CCAAAGATTCCACTTACAGTCTGTCCAGCACATTGACACTGAGTA | |
| | AGGCTGATTACGAAAAACACAAGGTGTACGCATGCGAGGTGACA | |
| | CACCAAGGTCTTTCATCTCCTGTAACTAAGAGCTTTAACCGGGGA | |
| | GAATGTGGTGGTGGGGGCAGCGGGGGCGGAGGTAGTGGAGGCGG | |
| | CGGTAGTGAACCAAAGAGTAGTGACAAAACTCACACGTGCCCAC | |
| | CGTGCCCAGCACCTGAACTCCTGGGGGGACCGTCAGTCTTCCTCTT | |
| | CCCCCCAAAACCCAAGGACACCCTCATGATCTCCCGGACCCCTGA | |
| | GGTCACATGCGTGGTGGTGGACGTGAGCCACGAAGACCCTGAGGT | |
| | CAAGTTCAACTGGTACGTGGACGGCGTGGAGGTGCATAATGCCAA | |
| | GACAAAGCCGCGGGAGGAGCAGTACAACAGCACGTACCGTGTGG | |
| | TCAGCGTCCTCACCGTCCTGCACCAGGACTGGCTGAATGGCAAGG | |
| | AGTACAAGTGCAAGGTCTCCAACAAAGCCCTCCCAGCCCCCATCG | |
| | AGAAAACCATCTCCAAAGCCAAAGGGCAGCCCCGAGAACCACAG | |
| | GTGTACACCCTGCCCCCATGCCGGGAGGAGATGACCAAGAACCA | |
| | GGTCAGCCTGTGGTGCCTGGTCAAAGGCTTCTATCCCAGCGACAT | |
| | CGCCGTGGAGTGGGAGAGCAATGGGCAGCCGGAGAACAACTACA | |
| | AGACCACGCCTCCCGTGCTGGACTCCGACGGCTCCTTCTTCCTCTA | |
| | CAGCAAGCTCACCGTGGACAAGAGCAGGTGGCAGCAGGGGAACG | |
| | TCTTCTCATGCTCCGTGATGCATGAGGCTCTGCACAACCACTACAC | |
| | GCAGAAGAGCCTCTCCCTGTCTCCGGGTAAA | |
| GPB01 HC | CAGGTTCAGCTTGTGCAATCAGGCGCTGAAGTCAAAAAACCAGGA | 510 |
| | GCAAGCGTGAAAGTAAGCTGTAAGGCTTCCGGTTACACTTTCACC | |
| | GATTACGAAATGCACTGGGTACGCCAGGCTCCTGGACAAGGCCTG | |
| | GAATGGATGGGCGCACTTGATCCAAAAACAGGTGACACTGCTTAC | |
| | AGTCAAAAATTCAAAGGTCGTGTCACTCTTACAGCAGATAAGTCC | |
| | ACCAGTACCGCTTATATGGAGCTTAGCTCCCTGACTTCCGAGGAC | |
| | ACCGCCGTGTATTATTGTACAAGATTCTACTCATATACTTACTGGG | |
| | GCCAAGGAACCCTGGTGACAGTGTCATCTGCTAGCACCAAAGGAC | |
| | CTAGTGTTTTTCCTCTTGCCCCTGAAGTTCAGCTTGTCGAAAGTGG | |
| | CGGAGGTCTTGTAAAGCCAGGAGGGTCCCTGAAGTTGAGTTGTGC | |
| | TGCCTCTGGCTTCACCTTTTCACGGTATGCCATGTCCTGGGTTCGA | |
| | CAGATACCTGAGAAGTGTCTTGAATGGGTGGCCGCAATAGACAGT | |
| | AGCGGTGGTGACACCTATTACCTCGACACAGTCAAAGACCGCTTT | |
| | ACTATCTCACGCGATAACGCCAACAATACCCTGCACTTGCAGATG | |
| | CGATCACTTCGTTCAGAAGACACTGCTCTTTACTATTGTGTACGCC | |
| | AAGGGGGAGCATACTGGGGTCAGGGAACACTGGTTACCGTGTCTT | |
| | CAGCTAGCACCAAAGGACCTAGTGTTTTTCCTCTTGCCCCTTCCTC | |
| | AAAGTCTACCTCTGGGGGGACAGCCGCTCTGGGCTGCCTGGTCAA | |
| | GGATTATTTCCCAGAGCCTGTCACTGTCAGTTGGAACTCTGGAGC | |
| | CTTGACTTCTGGTGTTCATACATTTCCTGCTGTCCTTCAAAGCAGC | |
| | GGCTTGTACTCATTGTCTTCTGTTGTGACAGTACCCTCAAGCAGCC | |
| | TCGGCACTCAGACATACATCTGCAATGTCAACCACAAACCCTCAA | |
| | ATACAAAGGTAGATAAAAAAGTCGAACCAAAGTCTTGTGACAAA | |
| | ACTCACACGTGCCCACCGTGCCCAGCACCTGAACTCCTGGGGGGA | |
| | CCGTCAGTCTTCCTCTTCCCCCCAAAACCCAAGGACACCCTCATGA | |
| | TCTCCCGGACCCCTGAGGTCACATGCGTGGTGGTGGACGTGAGCC | |
| | ACGAAGACCCTGAGGTCAAGTTCAACTGGTACGTGGACGGCGTGG | |
| | AGGTGCATAATGCCAAGACAAAGCCGCGGGAGGAGCAGTACAAC | |
| | AGCACGTACCGTGTGGTCAGCGTCCTCACCGTCCTGCACCAGGAC | |
| | TGGCTGAATGGCAAGGAGTACAAGTGCAAGGTCTCCAACAAAGC | |
| | CCTCCCAGCCCCCATCGAGAAAACCATCTCCAAAGCCAAAGGGCA | |
| | GCCCCGAGAACCACAGGTGTACACCCTGCCCCCATGCCGGGAGGA | |
| | GATGACCAAGAACCAGGTCAGCCTGTGGTGCCTGGTCAAAGGCTT | |
| | CTATCCCAGCGACATCGCCGTGGAGTGGGAGAGCAATGGGCAGC | |
| | CGGAGAACAACTACAAGACCACGCCTCCCGTGCTGGACTCCGACG | |
| | GCTCCTTCTTCCTCTACAGCAAGCTCACCGTGGACAAGAGCAGGT | |
| | GGCAGCAGGGGAACGTCTTCTCATGCTCCGTGATGCATGAGGCTC | |
| | TGCACAACCACTACACGCAGAAGAGCCTCTCCCTGTCTCCGGGTA | |
| | AA | |
| GPB01 LC | GATGTTGTTATGACTCAGTCACCTCTCTCACTTCCTGTAACCCCTG | 511 |
| | GTGAACCAGCCTCTATAAGCTGCCGGTCAAGTCAAAGCCTGGTTC | |
| | ATAGCAACCGTAACACTTACCTTCACTGGTACTTGCAAAAACCTG | |
| | GTCAGTCCCCACAACTCTTGATCTACAAAGTCTCCAATCGCTTCTC | |
| | TGGAGTCCCTGACAGGTTTTCTGGTAGTGGATCAGGTACAGACTT | |
| | CACTTTGAAAATCAGCCGCGTTGAGGCCGAGGACGTGGGAGTGTA | |
| | TTATTGCTCTCAGAATACCCATGTACCCCCAACCTTTGGCCAAGGG | |
| | ACTAAACTCGAGATTAAACGTACGGTGGCCGCTCCCTCCGTGTTC | |
| | ATCTTCCCACCCGACGTGGTTATGACTCAGACTCCTCTGACTCTCT | |
| | CCGTCACTATTGGTCAGCCCGCTTCCATATCATGTAAATCATCACA | |
| | ATCTCTTCTTGATAGCGATGGCAAGACTTATTTGAACTGGTTGTTG | |
| | CAACGCCCAGGTCAGAGCCCTAAGAGACTTATCTATTTGGTGAGC | |
| | AAACTCGACAGCGGTGCACCCGATCGTTTTACCGGAAGCGGCAGC | |
| | GGCACCGATTTCACACTGAAGATCAGTAGGGTCGAAGCTGAAGAC | |
| | CTGGGAATCTACTACTGCTGGCAAGGTACTCACTTTCCCCTGACTT | |
| | TCGGCTGCGGTACTAAACTTGAGATCAAACGTACGGTGGCAGCTC | |
| | CCAGCGTTTTTATCTTTCCCCCATCCGACGAGCAGCTCAAGAGTGG | |
| | CACTGCCTCTGTAGTTTGTTTGCTGAATAACTTCTATCCACGTGAA | |
| | GCAAAAGTACAGTGGAAGGTCGATAATGCCCTTCAGAGCGGTAA | |
| | CAGCCAAGAAAGTGTTACCGAGCAAGATTCCAAAGATTCCACTTA | |
| | CAGTCTGTCCAGCACATTGACACTGAGTAAGGCTGATTACGAAAA | |
| | ACACAAGGTGTACGCATGCGAGGTGACACACCAAGGTCTTTCATC | |
| | TCCTGTAACTAAGAGCTTTAACCGGGGAGAATGTGGTGGTGGGGG | |
| | CAGCGGGGGCGGAGGTAGTGGAGGCGGCGGTAGTGAACCAAAGA | |
| | GTAGTGACAAAACTCACACGTGCCCACCGTGCCCAGCACCTGAAC | |
| | TCCTGGGGGGACCGTCAGTCTTCCTCTTCCCCCCAAAACCCAAGG | |
| | ACACCCTCATGATCTCCCGGACCCCTGAGGTCACATGCGTGGTGG | |
| | TGGACGTGAGCCACGAAGACCCTGAGGTCAAGTTCAACTGGTACG | |
| | TGGACGGCGTGGAGGTGCATAATGCCAAGACAAAGCCGCGGGAG | |
| | GAGCAGTACAACAGCACGTACCGTGTGGTCAGCGTCCTCACCGTC | |
| | CTGCACCAGGACTGGCTGAATGGCAAGGAGTACAAGTGCAAGGT | |
| | CTCCAACAAAGCCCTCCCAGCCCCCATCGAGAAAACCATCTCCAA | |
| | AGCCAAAGGGCAGCCCCGAGAACCACAGGTGTACACCCTGCCCC | |
| | CATGCCGGGAGGAGATGACCAAGAACCAGGTCAGCCTGTGGTGC | |
| | CTGGTCAAAGGCTTCTATCCCAGCGACATCGCCGTGGAGTGGGAG | |
| | AGCAATGGGCAGCCGGAGAACAACTACAAGACCACGCCTCCCGT | |
| | GCTGGACTCCGACGGCTCCTTCTTCCTCTACAGCAAGCTCACCGTG | |
| | GACAAGAGCAGGTGGCAGCAGGGGAACGTCTTCTCATGCTCCGTG | |
| | ATGCATGAGGCTCTGCACAACCACTACACGCAGAAGAGCCTCTCC | |
| | CTGTCTCCGGGTAAA | |
| GPB03 HC | CAGGTTCAGCTTGTGCAATCAGGCGCTGAAGTCAAAAAACCAGGA | 512 |
| | GCAAGCGTGAAAGTAAGCTGTAAGGCTTCCGGTTACACTTTCACC | |
| | GATTACGAAATGCACTGGGTACGCCAGGCTCCTGGACAAGGCCTG | |
| | GAATGGATGGGCGCACTTGATCCAAAAACAGGTGACACTGCTTAC | |
| | AGTCAAAAATTCAAAGGTCGTGTCACTCTTACAGCAGATAAGTCC | |
| | ACCAGTACCGCTTATATGGAGCTTAGCTCCCTGACTTCCGAGGAC | |
| | ACCGCCGTGTATTATTGTACAAGATTCTACTCATATACTTACTGGG | |
| | GCCAAGGAACCCTGGTGACAGTGTCATCTGCTAGCACCAAAGGAC | |
| | CTAGTGTTTTTCCTCTTGCCCCTGAAGTGCAACTGGTCGAAACAGG | |
| | CGGGGGACTGGTACAGCCCAAGGGATCTTTGAAACTTAGTTGTGC | |
| | TGCTAGTGGGTTTACATTCAATGCCTCCGCAATGAACTGGGTAAG | |
| | ACAAGCTCCTGGCAAGTGCCTGGAATGGGTGGCCCGTATTCGCTC | |
| | TAAAAGTAATAACTACGCTATTTATTACGCTGATTCTGTAAAGGAT | |
| | CGGTTTACAATAAGTCGGGACGACAGCCAATCCATGCTGTATCTC | |
| | CAAATGAATAACCTGAAAACAGAGGATACTGCCATGTACTATTGT | |
| | GTGCGGGACCCAGGCTATTACGGGAATCCCTGGTTCGCCTATTGG | |
| | GGACAGGGCACTCTGGTTACCGTATCATCAGCTAGCACCAAAGGA | |
| | CCTAGTGTTTTTCCTCTTGCCCCTTCCTCAAAGTCTACCTCTGGGG | |
| | GGACAGCCGCTCTGGGCTGCCTGGTCAAGGATTATTTCCCAGAGC | |
| | CTGTCACTGTCAGTTGGAACTCTGGAGCCTTGACTTCTGGTGTTCA | |
| | TACATTTCCTGCTGTCCTTCAAAGCAGCGGCTTGTACTCATTGTCT | |
| | TCTGTTGTGACAGTACCCTCAAGCAGCCTCGGCACTCAGACATAC | |
| | ATCTGCAATGTCAACCACAAACCCTCAAATACAAAGGTAGATAAA | |
| | AAAGTCGAACCAAAGTCTTGTGACAAAACTCACACGTGCCCACCG | |
| | TGCCCAGCACCTGAACTCCTGGGGGGACCGTCAGTCTTCCTCTTCC | |
| | CCCCAAAACCCAAGGACACCCTCATGATCTCCCGGACCCCTGAGG | |
| | TCACATGCGTGGTGGTGGACGTGAGCCACGAAGACCCTGAGGTCA | |
| | AGTTCAACTGGTACGTGGACGGCGTGGAGGTGCATAATGCCAAGA | |
| | CAAAGCCGCGGGAGGAGCAGTACAACAGCACGTACCGTGTGGTC | |
| | AGCGTCCTCACCGTCCTGCACCAGGACTGGCTGAATGGCAAGGAG | |
| | TACAAGTGCAAGGTCTCCAACAAAGCCCTCCCAGCCCCCATCGAG | |
| | AAAACCATCTCCAAAGCCAAAGGGCAGCCCCGAGAACCACAGGT | |
| | GTACACCCTGCCCCCATGCCGGGAGGAGATGACCAAGAACCAGG | |
| | TCAGCCTGTGGTGCCTGGTCAAAGGCTTCTATCCCAGCGACATCG | |
| | CCGTGGAGTGGGAGAGCAATGGGCAGCCGGAGAACAACTACAAG | |
| | ACCACGCCTCCCGTGCTGGACTCCGACGGCTCCTTCTTCCTCTACA | |
| | GCAAGCTCACCGTGGACAAGAGCAGGTGGCAGCAGGGGAACGTC | |
| | TTCTCATGCTCCGTGATGCATGAGGCTCTGCACAACCACTACACG | |
| | CAGAAGAGCCTCTCCCTGTCTCCGGGTAAA | |
| GPB03 LC | GATGTTGTTATGACTCAGTCACCTCTCTCACTTCCTGTAACCCCTG | 513 |
| | GTGAACCAGCCTCTATAAGCTGCCGGTCAAGTCAAAGCCTGGTTC | |
| | ATAGCAACCGTAACACTTACCTTCACTGGTACTTGCAAAAACCTG | |
| | GTCAGTCCCCACAACTCTTGATCTACAAAGTCTCCAATCGCTTCTC | |
| | TGGAGTCCCTGACAGGTTTTCTGGTAGTGGATCAGGTACAGACTT | |
| | CACTTTGAAAATCAGCCGCGTTGAGGCCGAGGACGTGGGAGTGTA | |
| | TTATTGCTCTCAGAATACCCATGTACCCCCAACCTTTGGCCAAGGG | |
| | ACTAAACTCGAGATTAAACGTACGGTGGCCGCTCCCTCCGTGTTC | |
| | ATCTTCCCACCCCAAATTGTCCTCACCCAATCACCAGCTATAATGT | |
| | CTGCTTTTCCCGGCGAGAAAGTAACCATGACTTGTAGCGCCTCTA | |
| | GTAGCGTGTCATATATGTATTGGTATCAACAAAAGAGCGGTAGTT | |
| | CACCTCGACTCCTTATCTACGACACAAGTAATCTCGCTAGTGGTGT | |
| | CCCAGTCCGTTTCTCCGGGAGCGGCAGCGGCACATCATACTCCCT | |
| | GACCATCTCCAGAATGGAGGCCGAGGACGCTGCCACATACTATTG | |
| | TCAGCAGTGGAGCTCATATCCTTTGACATTCGGTTGCGGTACTAAA | |
| | CTCGAAATCAAGCGTACGGTGGCAGCTCCCAGCGTTTTTATCTTTC | |
| | CCCCATCCGACGAGCAGCTCAAGAGTGGCACTGCCTCTGTAGTTT | |
| | GTTTGCTGAATAACTTCTATCCACGTGAAGCAAAAGTACAGTGGA | |
| | AGGTCGATAATGCCCTTCAGAGCGGTAACAGCCAAGAAAGTGTTA | |
| | CCGAGCAAGATTCCAAAGATTCCACTTACAGTCTGTCCAGCACAT | |
| | TGACACTGAGTAAGGCTGATTACGAAAAACACAAGGTGTACGCAT | |
| | GCGAGGTGACACACCAAGGTCTTTCATCTCCTGTAACTAAGAGCT | |
| | TTAACCGGGGAGAATGTGGTGGTGGGGGCAGCGGGGGCGGAGGT | |
| | AGTGGAGGCGGCGGTAGTGAACCAAAGAGTAGTGACAAAACTCA | |
| | CACGTGCCCACCGTGCCCAGCACCTGAACTCCTGGGGGGACCGTC | |
| | AGTCTTCCTCTTCCCCCCAAAACCCAAGGACACCCTCATGATCTCC | |
| | CGGACCCCTGAGGTCACATGCGTGGTGGTGGACGTGAGCCACGAA | |
| | GACCCTGAGGTCAAGTTCAACTGGTACGTGGACGGCGTGGAGGTG | |
| | CATAATGCCAAGACAAAGCCGCGGGAGGAGCAGTACAACAGCAC | |
| | GTACCGTGTGGTCAGCGTCCTCACCGTCCTGCACCAGGACTGGCT | |
| | GAATGGCAAGGAGTACAAGTGCAAGGTCTCCAACAAAGCCCTCC | |
| | CAGCCCCCATCGAGAAAACCATCTCCAAAGCCAAAGGGCAGCCC | |
| | CGAGAACCACAGGTGTACACCCTGCCCCCATGCCGGGAGGAGAT | |
| | GACCAAGAACCAGGTCAGCCTGTGGTGCCTGGTCAAAGGCTTCTA | |
| | TCCCAGCGACATCGCCGTGGAGTGGGAGAGCAATGGGCAGCCGG | |
| | AGAACAACTACAAGACCACGCCTCCCGTGCTGGACTCCGACGGCT | |
| | CCTTCTTCCTCTACAGCAAGCTCACCGTGGACAAGAGCAGGTGGC | |
| | AGCAGGGGAACGTCTTCTCATGCTCCGTGATGCATGAGGCTCTGC | |
| | ACAACCACTACACGCAGAAGAGCCTCTCCCTGTCTCCGGGTAAA | |
| GPB04 HC | GAAGTTCAGCTTGTCGAAAGTGGCGGAGGTCTTGTAAAGCCAGGA | 514 |
| | GGGTCCCTGAAGTTGAGTTGTGCTGCCTCTGGCTTCACCTTTTCAC | |
| | GGTATGCCATGTCCTGGGTTCGACAGATACCTGAGAAGATTCTTG | |
| | AATGGGTGGCCGCAATAGACAGTAGCGGTGGTGACACCTATTACC | |
| | TCGACACAGTCAAAGACCGCTTTACTATCTCACGCGATAACGCCA | |
| | ACAATACCCTGCACTTGCAGATGCGATCACTTCGTTCAGAAGACA | |
| | CTGCTCTTTACTATTGTGTACGCCAAGGGGGAGCATACTGGGGTC | |
| | AGGGAACACTGGTTACCGTGTCTTCAGCTAGCACCAAAGGACCTA | |
| | GTGTTTTTCCTCTTGCCCCTCAGGTTCAGCTTGTGCAATCAGGCGC | |
| | TGAAGTCAAAAAACCAGGAGCAAGCGTGAAAGTAAGCTGTAAGG | |
| | CTTCCGGTTACACTTTCACCGATTACGAAATGCACTGGGTACGCC | |
| | AGGCTCCTGGACAATGTCTGGAATGGATGGGCGCACTTGATCCAA | |
| | AAACAGGTGACACTGCTTACAGTCAAAAATTCAAAGGTCGTGTCA | |
| | CTCTTACAGCAGATAAGTCCACCAGTACCGCTTATATGGAGCTTA | |
| | GCTCCCTGACTTCCGAGGACACCGCCGTGTATTATTGTACAAGATT | |
| | CTACTCATATACTTACTGGGGCCAAGGAACCCTGGTGACAGTGTC | |
| | ATCTGCTAGCACCAAAGGACCTAGTGTTTTTCCTCTTGCCCCTTCC | |
| | TCAAAGTCTACCTCTGGGGGGACAGCCGCTCTGGGCTGCCTGGTC | |
| | AAGGATTATTTCCCAGAGCCTGTCACTGTCAGTTGGAACTCTGGA | |
| | GCCTTGACTTCTGGTGTTCATACATTTCCTGCTGTCCTTCAAAGCA | |
| | GCGGCTTGTACTCATTGTCTTCTGTTGTGACAGTACCCTCAAGCAG | |
| | CCTCGGCACTCAGACATACATCTGCAATGTCAACCACAAACCCTC | |
| | AAATACAAAGGTAGATAAAAAAGTCGAACCAAAGTCTTGTGACA | |
| | AAACTCACACGTGCCCACCGTGCCCAGCACCTGAACTCCTGGGGG | |
| | GACCGTCAGTCTTCCTCTTCCCCCCAAAACCCAAGGACACCCTCAT | |
| | GATCTCCCGGACCCCTGAGGTCACATGCGTGGTGGTGGACGTGAG | |
| | CCACGAAGACCCTGAGGTCAAGTTCAACTGGTACGTGGACGGCGT | |
| | GGAGGTGCATAATGCCAAGACAAAGCCGCGGGAGGAGCAGTACA | |
| | ACAGCACGTACCGTGTGGTCAGCGTCCTCACCGTCCTGCACCAGG | |
| | ACTGGCTGAATGGCAAGGAGTACAAGTGCAAGGTCTCCAACAAA | |
| | GCCCTCCCAGCCCCCATCGAGAAAACCATCTCCAAAGCCAAAGGG | |
| | CAGCCCCGAGAACCACAGGTGTACACCCTGCCCCCATGCCGGGAG | |
| | GAGATGACCAAGAACCAGGTCAGCCTGTGGTGCCTGGTCAAAGG | |
| | CTTCTATCCCAGCGACATCGCCGTGGAGTGGGAGAGCAATGGGCA | |
| | GCCGGAGAACAACTACAAGACCACGCCTCCCGTGCTGGACTCCGA | |
| | CGGCTCCTTCTTCCTCTACAGCAAGCTCACCGTGGACAAGAGCAG | |
| | GTGGCAGCAGGGGAACGTCTTCTCATGCTCCGTGATGCATGAGGC | |
| | TCTGCACAACCACTACACGCAGAAGAGCCTCTCCCTGTCTCCGGG | |
| | TAAA | |
| GPB04 LC | GACGTGGTTATGACTCAGACTCCTCTGACTCTCTCCGTCACTATTG | 515 |
| | GTCAGCCCGCTTCCATATCATGTAAATCATCACAATCTCTTCTTGA | |
| | TAGCGATGGCAAGACTTATTTGAACTGGTTGTTGCAACGCCCAGG | |
| | TCAGAGCCCTAAGAGACTTATCTATTTGGTGAGCAAACTCGACAG | |
| | CGGTGCACCCGATCGTTTTACCGGAAGCGGCAGCGGCACCGATTT | |
| | CACACTGAAGATCAGTAGGGTCGAAGCTGAAGACCTGGGAATCT | |
| | ACTACTGCTGGCAAGGTACTCACTTTCCCCTGACTTTCGGCGCCGG | |
| | TACTAAACTTGAGATCAAACGTACGGTGGCAGCTCCCAGCGTTTT | |
| | TATCTTTCCCCCAGATGTTGTTATGACTCAGTCACCTCTCTCACTTC | |
| | CTGTAACCCCTGGTGAACCAGCCTCTATAAGCTGCCGGTCAAGTC | |
| | AAAGCCTGGTTCATAGCAACCGTAACACTTACCTTCACTGGTACTT | |
| | GCAAAAACCTGGTCAGTCCCCACAACTCTTGATCTACAAAGTCTC | |
| | CAATCGCTTCTCTGGAGTCCCTGACAGGTTTTCTGGTAGTGGATCA | |
| | GGTACAGACTTCACTTTGAAAATCAGCCGCGTTGAGGCCGAGGAC | |
| | GTGGGAGTGTATTATTGCTCTCAGAATACCCATGTACCCCCAACCT | |
| | TTGGCTGTGGGACTAAACTCGAGATTAAACGTACGGTGGCAGCTC | |
| | CCAGCGTTTTTATCTTTCCCCCATCCGACGAGCAGCTCAAGAGTGG | |
| | CACTGCCTCTGTAGTTTGTTTGCTGAATAACTTCTATCCACGTGAA | |
| | GCAAAAGTACAGTGGAAGGTCGATAATGCCCTTCAGAGCGGTAA | |
| | CAGCCAAGAAAGTGTTACCGAGCAAGATTCCAAAGATTCCACTTA | |
| | CAGTCTGTCCAGCACATTGACACTGAGTAAGGCTGATTACGAAAA | |
| | ACACAAGGTGTACGCATGCGAGGTGACACACCAAGGTCTTTCATC | |
| | TCCTGTAACTAAGAGCTTTAACCGGGGAGAATGTGGTGGTGGGGG | |
| | CAGCGGGGGCGGAGGTAGTGGAGGCGGCGGTAGTGAACCAAAGA | |
| | GTAGTGACAAAACTCACACGTGCCCACCGTGCCCAGCACCTGAAC | |
| | TCCTGGGGGGACCGTCAGTCTTCCTCTTCCCCCCAAAACCCAAGG | |
| | ACACCCTCATGATCTCCCGGACCCCTGAGGTCACATGCGTGGTGG | |
| | TGGACGTGAGCCACGAAGACCCTGAGGTCAAGTTCAACTGGTACG | |
| | TGGACGGCGTGGAGGTGCATAATGCCAAGACAAAGCCGCGGGAG | |
| | GAGCAGTACAACAGCACGTACCGTGTGGTCAGCGTCCTCACCGTC | |
| | CTGCACCAGGACTGGCTGAATGGCAAGGAGTACAAGTGCAAGGT | |
| | CTCCAACAAAGCCCTCCCAGCCCCCATCGAGAAAACCATCTCCAA | |
| | AGCCAAAGGGCAGCCCCGAGAACCACAGGTGTACACCCTGCCCC | |
| | CATGCCGGGAGGAGATGACCAAGAACCAGGTCAGCCTGTGGTGC | |
| | CTGGTCAAAGGCTTCTATCCCAGCGACATCGCCGTGGAGTGGGAG | |
| | AGCAATGGGCAGCCGGAGAACAACTACAAGACCACGCCTCCCGT | |
| | GCTGGACTCCGACGGCTCCTTCTTCCTCTACAGCAAGCTCACCGTG | |
| | GACAAGAGCAGGTGGCAGCAGGGGAACGTCTTCTCATGCTCCGTG | |
| | ATGCATGAGGCTCTGCACAACCACTACACGCAGAAGAGCCTCTCC | |
| | CTGTCTCCGGGTAAA | |
| GPB06 HC | GAAGTGCAACTGGTCGAAACAGGCGGGGGACTGGTACAGCCCAA | 516 |
| | GGGATCTTTGAAACTTAGTTGTGCTGCTAGTGGGTTTACATTCAAT | |
| | GCCTCCGCAATGAACTGGGTAAGACAAGCTCCTGGCAAGGGCCTG | |
| | GAATGGGTGGCCCGTATTCGCTCTAAAAGTAATAACTACGCTATT | |
| | TATTACGCTGATTCTGTAAAGGATCGGTTTACAATAAGTCGGGAC | |
| | GACAGCCAATCCATGCTGTATCTCCAAATGAATAACCTGAAAACA | |
| | GAGGATACTGCCATGTACTATTGTGTGCGGGACCCAGGCTATTAC | |
| | GGGAATCCCTGGTTCGCCTATTGGGGACAGGGCACTCTGGTTACC | |
| | GTATCATCAGCTAGCACCAAAGGACCTAGTGTTTTTCCTCTTGCCC | |
| | CTCAGGTTCAGCTTGTGCAATCAGGCGCTGAAGTCAAAAAACCAG | |
| | GAGCAAGCGTGAAAGTAAGCTGTAAGGCTTCCGGTTACACTTTCA | |
| | CCGATTACGAAATGCACTGGGTACGCCAGGCTCCTGGACAATGTC | |
| | TGGAATGGATGGGCGCACTTGATCCAAAAACAGGTGACACTGCTT | |
| | ACAGTCAAAAATTCAAAGGTCGTGTCACTCTTACAGCAGATAAGT | |
| | CCACCAGTACCGCTTATATGGAGCTTAGCTCCCTGACTTCCGAGG | |
| | ACACCGCCGTGTATTATTGTACAAGATTCTACTCATATACTTACTG | |
| | GGGCCAAGGAACCCTGGTGACAGTGTCATCTGCTAGCACCAAAGG | |
| | ACCTAGTGTTTTTCCTCTTGCCCCTTCCTCAAAGTCTACCTCTGGG | |
| | GGGACAGCCGCTCTGGGCTGCCTGGTCAAGGATTATTTCCCAGAG | |
| | CCTGTCACTGTCAGTTGGAACTCTGGAGCCTTGACTTCTGGTGTTC | |
| | ATACATTTCCTGCTGTCCTTCAAAGCAGCGGCTTGTACTCATTGTC | |
| | TTCTGTTGTGACAGTACCCTCAAGCAGCCTCGGCACTCAGACATA | |
| | CATCTGCAATGTCAACCACAAACCCTCAAATACAAAGGTAGATAA | |
| | AAAAGTCGAACCAAAGTCTTGTGACAAAACTCACACGTGCCCACC | |
| | GTGCCCAGCACCTGAACTCCTGGGGGGACCGTCAGTCTTCCTCTTC | |
| | CCCCCAAAACCCAAGGACACCCTCATGATCTCCCGGACCCCTGAG | |
| | GTCACATGCGTGGTGGTGGACGTGAGCCACGAAGACCCTGAGGTC | |
| | AAGTTCAACTGGTACGTGGACGGCGTGGAGGTGCATAATGCCAAG | |
| | ACAAAGCCGCGGGAGGAGCAGTACAACAGCACGTACCGTGTGGT | |
| | CAGCGTCCTCACCGTCCTGCACCAGGACTGGCTGAATGGCAAGGA | |
| | GTACAAGTGCAAGGTCTCCAACAAAGCCCTCCCAGCCCCCATCGA | |
| | GAAAACCATCTCCAAAGCCAAAGGGCAGCCCCGAGAACCACAGG | |
| | TGTACACCCTGCCCCCATGCCGGGAGGAGATGACCAAGAACCAG | |
| | GTCAGCCTGTGGTGCCTGGTCAAAGGCTTCTATCCCAGCGACATC | |
| | GCCGTGGAGTGGGAGAGCAATGGGCAGCCGGAGAACAACTACAA | |
| | GACCACGCCTCCCGTGCTGGACTCCGACGGCTCCTTCTTCCTCTAC | |
| | AGCAAGCTCACCGTGGACAAGAGCAGGTGGCAGCAGGGGAACGT | |
| | CTTCTCATGCTCCGTGATGCATGAGGCTCTGCACAACCACTACAC | |
| | GCAGAAGAGCCTCTCCCTGTCTCCGGGTAAA | |
| GPB06 LC | CAAATTGTCCTCACCCAATCACCAGCTATAATGTCTGCTTTTCCCG | 517 |
| | GCGAGAAAGTAACCATGACTTGTAGCGCCTCTAGTAGCGTGTCAT | |
| | ATATGTATTGGTATCAACAAAAGAGCGGTAGTTCACCTCGACTCC | |
| | TTATCTACGACACAAGTAATCTCGCTAGTGGTGTCCCAGTCCGTTT | |
| | CTCCGGGAGCGGCAGCGGCACATCATACTCCCTGACCATCTCCAG | |
| | AATGGAGGCCGAGGACGCTGCCACATACTATTGTCAGCAGTGGAG | |
| | CTCATATCCTTTGACATTCGGTGGAGGTACTAAACTCGAAATCAA | |
| | GCGTACGGTGGCAGCTCCCAGCGTTTTTATCTTTCCCCCAGATGTT | |
| | GTTATGACTCAGTCACCTCTCTCACTTCCTGTAACCCCTGGTGAAC | |
| | CAGCCTCTATAAGCTGCCGGTCAAGTCAAAGCCTGGTTCATAGCA | |
| | ACCGTAACACTTACCTTCACTGGTACTTGCAAAAACCTGGTCAGT | |
| | CCCCACAACTCTTGATCTACAAAGTCTCCAATCGCTTCTCTGGAGT | |
| | CCCTGACAGGTTTTCTGGTAGTGGATCAGGTACAGACTTCACTTTG | |
| | AAAATCAGCCGCGTTGAGGCCGAGGACGTGGGAGTGTATTATTGC | |
| | TCTCAGAATACCCATGTACCCCCAACCTTTGGCTGTGGGACTAAA | |
| | CTCGAGATTAAACGTACGGTGGCAGCTCCCAGCGTTTTTATCTTTC | |
| | CCCCATCCGACGAGCAGCTCAAGAGTGGCACTGCCTCTGTAGTTT | |
| | GTTTGCTGAATAACTTCTATCCACGTGAAGCAAAAGTACAGTGGA | |
| | AGGTCGATAATGCCCTTCAGAGCGGTAACAGCCAAGAAAGTGTTA | |
| | CCGAGCAAGATTCCAAAGATTCCACTTACAGTCTGTCCAGCACAT | |
| | TGACACTGAGTAAGGCTGATTACGAAAAACACAAGGTGTACGCAT | |
| | GCGAGGTGACACACCAAGGTCTTTCATCTCCTGTAACTAAGAGCT | |
| | TTAACCGGGGAGAATGTGGTGGTGGGGGCAGCGGGGGCGGAGGT | |
| | AGTGGAGGCGGCGGTAGTGAACCAAAGAGTAGTGACAAAACTCA | |
| | CACGTGCCCACCGTGCCCAGCACCTGAACTCCTGGGGGGACCGTC | |
| | AGTCTTCCTCTTCCCCCCAAAACCCAAGGACACCCTCATGATCTCC | |
| | CGGACCCCTGAGGTCACATGCGTGGTGGTGGACGTGAGCCACGAA | |
| | GACCCTGAGGTCAAGTTCAACTGGTACGTGGACGGCGTGGAGGTG | |
| | CATAATGCCAAGACAAAGCCGCGGGAGGAGCAGTACAACAGCAC | |
| | GTACCGTGTGGTCAGCGTCCTCACCGTCCTGCACCAGGACTGGCT | |
| | GAATGGCAAGGAGTACAAGTGCAAGGTCTCCAACAAAGCCCTCC | |
| | CAGCCCCCATCGAGAAAACCATCTCCAAAGCCAAAGGGCAGCCC | |
| | CGAGAACCACAGGTGTACACCCTGCCCCCATGCCGGGAGGAGAT | |
| | GACCAAGAACCAGGTCAGCCTGTGGTGCCTGGTCAAAGGCTTCTA | |
| | TCCCAGCGACATCGCCGTGGAGTGGGAGAGCAATGGGCAGCCGG | |
| | AGAACAACTACAAGACCACGCCTCCCGTGCTGGACTCCGACGGCT | |
| | CCTTCTTCCTCTACAGCAAGCTCACCGTGGACAAGAGCAGGTGGC | |
| | AGCAGGGGAACGTCTTCTCATGCTCCGTGATGCATGAGGCTCTGC | |
| | ACAACCACTACACGCAGAAGAGCCTCTCCCTGTCTCCGGGTAAA | |

Table 46 below shows the polypeptide sequences of the heavy chain variable region (VH) and light chain variable region (VL) of the engineered antibodies targeting GPC-3. Table 47 below shows the nucleotide sequences of the heavy chain variable region (VH) and light chain variable region (VL) of the engineered antibodies targeting GPC-3.

TABLE 46

| | | | SEQ |
|---|---|---|---|
| Name | | Sequence | ID NO |
| GPM01 | VH | EVQLVESGGGLVKPGGSLKLSCAASGFTFSRYAMSWVRQIPEKCLEW | 81 |
| | | VAAIDSSGGDTYYLDTVKDRFTTSRDNANNTLHLQMRSLRSEDTALY | |
| | | YCVRQGGAYWGQGTLVTVSS | |
| | VL | DVVMTQTPLTLSVTTGQPASISCKSSQSLLDSDGKTYLNWLLQRPGQS | 82 |
| | | PKRLIYLVSKLDSGAPDRFTGSGSGTDFTLKISRVEAEDLGIYYCWQG | |
| | | THFPLTFGCGTKLEIK | |
| GPM02 | VH | QVQLVQSGAEVKKPGASVKVSCKASGYTFTDYEMHWVRQAPGQCL | 83 |
| | | EWMGALDPKTGDTAYSQKFKGRVTLTADKSTSTAYMELSSLTSEDT | |
| | | AVYYCTRFYSYTYWGQGTLVTVSS | |
| | VL | DVVMTQSPLSLPVTPGEPASISCRSSQSLVHSNRNTYLHWYLQKPGQS | 84 |
| | | PQLLIYKVSNRFSGVPDRFSGSGSGTDFTLKISRVEAEDVGVYYCSQN | |
| | | THVPPTFGCGTKLEIK | |
| GPM04 | VH | EVQLVETGGGLVQPKGSLKLSCAASGFTFNASAMNWVRQAPGKCLE | 85 |
| | | WVARIRSKSNNYAIYYADSVKDRFTTSRDDSQSMLYLQMNNLKTEDT | |
| | | AMYYCVRDPGYYGNPWFAYWGQGTLVTVSS | |
| | VL | QIVLTQSPAIMSAFPGEKVTMTCSASSSVSYMYWYQQKSGSSPRLLIY | 86 |
| | | DTSNLASGVPVRFSGSGSGTSYSLTTSRMEAEDAATYYCQQWSSYPLT | |
| | | FGCGTKLEIK | |
| GPB01 | VH1 | QVQLVQSGAEVKKPGASVKVSCKASGYTFTDYEMHWVRQAPGQGL | 87 |
| | | EWMGALDPKTGDTAYSQKFKGRVTLTADKSTSTAYMELSSLTSEDT | |
| | | AVYYCTRFYSYTYWGQGTLVTVSS | |
| | VH2 | EVQLVESGGGLVKPGGSLKLSCAASGFTFSRYAMSWVRQIPEKCLEW | 81 |
| | | VAAIDSSGGDTYYLDTVKDRFTTSRDNANNTLHLQMRSLRSEDTALY | |
| | | YCVRQGGAYWGQGTLVTVSS | |
| | VL1 | DVVMTQSPLSLPVTPGEPASISCRSSQSLVHSNRNTYLHWYLQKPGQS | 88 |
| | | PQLLIYKVSNRFSGVPDRFSGSGSGTDFTLKISRVEAEDVGVYYCSQN | |
| | | THVPPTFGQGTKLEIK | |
| | VL2 | DVVMTQTPLTLSVTTGQPASISCKSSQSLLDSDGKTYLNWLLQRPGQS | 82 |
| | | PKRLIYLVSKLDSGAPDRFTGSGSGTDFTLKISRVEAEDLGIYYCWQG | |
| | | THFPLTFGCGTKLEIK | |
| GPB03 | VH1 | QVQLVQSGAEVKKPGASVKVSCKASGYTFTDYEMHWVRQAPGQGL | 87 |
| | | EWMGALDPKTGDTAYSQKFKGRVTLTADKSTSTAYMELSSLTSEDT | |
| | | AVYYCTRFYSYTYWGQGTLVTVSS | |
| | VH2 | EVQLVETGGGLVQPKGSLKLSCAASGFTFNASAMNWVRQAPGKCLE | 85 |
| | | WVARIRSKSNNYAIYYADSVKDRFTTSRDDSQSMLYLQMNNLKTEDT | |
| | | AMYYCVRDPGYYGNPWFAYWGQGTLVTVSS | |
| | VL1 | DVVMTQSPLSLPVTPGEPASISCRSSQSLVHSNRNTYLHWYLQKPGQS | 88 |
| | | PQLLIYKVSNRFSGVPDRFSGSGSGTDFTLKISRVEAEDVGVYYCSQN | |
| | | THVPPTFGQGTKLEIK | |
| | VL2 | QIVLTQSPAIMSAFPGEKVTMTCSASSSVSYMYWYQQKSGSSPRLLIY | 86 |
| | | DTSNLASGVPVRFSGSGSGTSYSLTTSRMEAEDAATYYCQQWSSYPLT | |
| | | FGCGTKLEIK | |
| GPB04 | VH1 | EVQLVESGGGLVKPGGSLKLSCAASGFTFSRYAMSWVRQIPEKILEW | 89 |
| | | VAAIDSSGGDTYYLDTVKDRFTTSRDNANNTLHLQMRSLRSEDTALY | |
| | | YCVRQGGAYWGQGTLVTVSS | |
| | VH2 | QVQLVQSGAEVKKPGASVKVSCKASGYTFTDYEMHWVRQAPGQCL | 83 |
| | | EWMGALDPKTGDTAYSQKFKGRVTLTADKSTSTAYMELSSLTSEDT | |
| | | AVYYCTRFYSYTYWGQGTLVTVSS | |
| | VL1 | DVVMTQTPLTLSVTTGQPASISCKSSQSLLDSDGKTYLNWLLQRPGQS | 90 |
| | | PKRLIYLVSKLDSGAPDRFTGSGSGTDFTLKISRVEAEDLGIYYCWQG | |
| | | THFPLTFGAGTKLEIK | |
| | VL2 | DVVMTQSPLSLPVTPGEPASISCRSSQSLVHSNRNTYLHWYLQKPGQS | \|84 |
| | | PQLLIYKVSNRFSGVPDRFSGSGSGTDFTLKISRVEAEDVGVYYCSQN | |
| | | THVPPTFGCGTKLEIK | |
| GPB06 | VH1 | EVQLVETGGGLVQPKGSLKLSCAASGFTFNASAMNWVRQAPGKGLE | 91 |
| | | WVARIRSKSNNYAIYYADSVKDRFTTSRDDSQSMLYLQMNNLKTEDT | |
| | | AMYYCVRDPGYYGNPWFAYWGQGTLVTVSS | |
| | VH2 | QVQLVQSGAEVKKPGASVKVSCKASGYTFTDYEMHWVRQAPGQCL | 83 |
| | | EWMGALDPKTGDTAYSQKFKGRVTLTADKSTSTAYMELSSLTSEDT | |
| | | AVYYCTRFYSYTYWGQGTLVTVSS | |
| | VL1 | QIVLTQSPAIMSAFPGEKVTMTCSASSSVSYMYWYQQKSGSSPRLLIY | 92 |
| | | DTSNLASGVPVRFSGSGSGTSYSLTTSRMEAEDAATYYCQQWSSYPLT | |
| | | FGGGTKLEIK | |
| | VL2 | DVVMTQSPLSLPVTPGEPASISCRSSQSLVHSNRNTYLHWYLQKPGQS | 84 |
| | | PQLLIYKVSNRFSGVPDRFSGSGSGTDFTLKISRVEAEDVGVYYCSQN | |
| | | THVPPTFGCGTKLEIK | |

TABLE 47

| | | | SEQ |
|---|---|---|---|
| Name | | Sequence | ID NO |
| GPM01 | VH | GAAGTTCAGCTTGTCGAAAGTGGCGGAGGTCTTGTAAAGCCAGGAGGGTCCCTGAAGTTGA | 518 |
| | | GTTGTGCTGCCTCTGGCTTCACCTTTTCACGGTATGCCATGTCCTGGGTTCGACAGATACC | |
| | | TGAGAAGTGTCTTGAATGGGTGGCCGCAATAGACAGTAGCGGTGGTGACACCTATTACCTC | |
| | | GACACAGTCAAAGACCGCTTTACTATCTCACGCGATAACGCCAACAATACCCTGCACTTGC | |
| | | AGATGCGATCACTTCGTTCAGAAGACACTGCTCTTTACTATTGTGTACGCCAAGGGGGAGC | |
| | | ATACTGGGGTCAGGGAACACTGGTTACCGTGTCTTCA | |
| | VL | GACGTGGTTATGACTCAGACTCCTCTGACTCTCTCCGTCACTATTGGTCAGCCCGCTTCCA | 519 |
| | | TATCATGTAAATCATCACAATCTCTTCTTGATAGCGATGGCAAGACTTATTTGAACTGGTT | |
| | | GTTGCAACGCCCAGGTCAGAGCCCTAAGAGACTTATCTATTTGGTGAGCAAACTCGACAGC | |
| | | GGTGCACCCGATCGTTTTACCGGAAGCGGCAGCGGCACCGATTTCACACTGAAGATCAGTA | |
| | | GGGTCGAAGCTGAAGACCTGGGAATCTACTACTGCTGGCAAGGTACTCACTTTCCCCTGAC | |
| | | TTTCGGCTGCGGTACTAAACTTGAGATCAAA | |
| GPM02 | VH | CAGGTTCAGCTTGTGCAATCAGGCGCTGAAGTCAAAAAACCAGGAGCAAGCGTGAAAGTAA | 520 |
| | | GCTGTAAGGCTTCCGGTTACACTTTCACCGATTACGAAATGCACTGGGTACGCCAGGCTCC | |
| | | TGGACAATGTCTGGAATGGATGGGCGCACTTGATCCAAAAACAGGTGACACTGCTTACAGT | |
| | | CAAAAATTCAAAGGTCGTGTCACTCTTACAGCAGATAAGTCCACCAGTACCGCTTATATGG | |
| | | AGCTTAGCTCCCTGACTTCCGAGGACACCGCCGTGTATTATTGTACAAGATTCTACTCATA | |
| | | TACTTACTGGGGCCAAGGAACCCTGGTGACAGTGTCATCT | |
| | VL | GATGTTGTTATGACTCAGTCACCTCTCTCACTTCCTGTAACCCCTGGTGAACCAGCCTCTA | 521 |
| | | TAAGCTGCCGGTCAAGTCAAAGCCTGGTTCATAGCAACCGTAACACTTACCTTCACTGGTA | |
| | | CTTGCAAAAACCTGGTCAGTCCCCACAACTCTTGATCTACAAAGTCTCCAATCGCTTCTCT | |
| | | GGAGTCCCTGACAGGTTTTCTGGTAGTGGATCAGGTACAGACTTCACTTTGAAAATCAGCC | |
| | | GCGTTGAGGCCGAGGACGTGGGAGTGTATTATTGCTCTCAGAATACCCATGTACCCCCAAC | |
| | | CTTTGGCTGTGGGACTAAACTCGAGATTAAA | |
| GPM04 | VH | GAAGTGCAACTGGTCGAAACAGGCGGGGGACTGGTACAGCCCAAGGGATCTTTGAAACTTA | 522 |
| | | GTTGTGCTGCTAGTGGGTTTACATTCAATGCCTCCGCAATGAACTGGGTAAGACAAGCTCC | |
| | | TGGCAAGTGCCTGGAATGGGTGGCCCGTATTCGCTCTAAAAGTAATAACTACGCTATTTAT | |
| | | TACGCTGATTCTGTAAAGGATCGGTTTACAATAAGTCGGGACGACAGCCAATCCATGCTGT | |
| | | ATCTCCAAATGAATAACCTGAAAACAGAGGATACTGCCATGTACTATTGTGTGCGGGACCC | |
| | | AGGCTATTACGGGAATCCCTGGTTCGCCTATTGGGGACAGGGCACTCTGGTTACCGTATCA | |
| | | TCA | |
| | VL | CAAATTGTCCTCACCCAATCACCAGCTATAATGTCTGCTTTTCCCGGCGAGAAAGTAACCA | 523 |
| | | TGACTTGTAGCGCCTCTAGTAGCGTGTCATATATGTATTGGTATCAACAAAAGAGCGGTAG | |
| | | TTCACCTCGACTCCTTATCTACGACACAAGTAATCTCGCTAGTGGTGTCCCAGTCCGTTTC | |
| | | TCCGGGAGCGGCAGCGGCACATCATACTCCCTGACCATCTCCAGAATGGAGGCCGAGGACG | |
| | | CTGCCACATACTATTGTCAGCAGTGGAGCTCATATCCTTTGACATTCGGTTGCGGTACTAA | |
| | | ACTCGAAATCAAG | |
| GPB01 | VH1 | CAGGTGCAACTCGTTCAAAGCGGGGCCGAGGTGAAGAAACCAGGGGCCTCAGTTAAGGTGA | 434 |
| | | GTTGCAAGGCAAGTGGATACACTTTCACCGATTATGAAATGCATTGGGTGCGTCAGGCCCC | |
| | | AGGACAAGGACTGGAGTGGATGGGCGCTCTCGATCCTAAGACTGGTGATACTGCTTACTCT | |
| | | CAAAAGTTCAAAGGCCGAGTCACCTTGACCGCCGACAAGTCCACATCCACTGCATATATGG | |
| | | AATTGTCAAGTCTGACAAGCGAAGATACAGCCGTCTACTACTGCACCCGCTTTTATAGCTA | |
| | | TACATATTGGGGACAGGGGACCTTGGTTACTGTGTCATCT | |
| | VH2 | GAAGTTCAGCTTGTCGAAAGTGGCGGAGGTCTTGTAAAGCCAGGAGGGTCCCTGAAGTTGA | 518 |
| | | GTTGTGCTGCCTCTGGCTTCACCTTTTCACGGTATGCCATGTCCTGGGTTCGACAGATACC | |
| | | TGAGAAGTGTCTTGAATGGGTGGCCGCAATAGACAGTAGCGGTGGTGACACCTATTACCTC | |
| | | GACACAGTCAAAGACCGCTTTACTATCTCACGCGATAACGCCAACAATACCCTGCACTTGC | |
| | | AGATGCGATCACTTCGTTCAGAAGACACTGCTCTTTACTATTGTGTACGCCAAGGGGGAGC | |
| | | ATACTGGGGTCAGGGAACACTGGTTACCGTGTCTTCA | |
| | VL1 | GACGTGGTAATGACACAATCACCTTTGTCTCTTCCCGTAACCCCCGGTGAACCAGCCAGCA | 435 |
| | | TCTCATGCAGAAGCAGTCAGTCACTGGTACATTCCAACCGTAATACTTATCTTCACTGGTA | |
| | | CTTGCAGAAGCCTGGGCAGTCTCCTCAACTTTTGATATATAAAGTGAGCAATCGGTTTAGC | |
| | | GGTGTCCCAGACCGCTTTTCTGGATCTGGAAGTGGAACAGACTTTACTCTGAAAATAAGCA | |
| | | GAGTCGAGGCAGAAGATGTCGGAGTTTACTACTGTAGCCAGAACACACACGTACCCCCAAC | |
| | | CTTTGGACAGGGCACAAAGTTGGAAATCAAG | |
| | VL2 | GACGTGGTTATGACTCAGACTCCTCTGACTCTCTCCGTCACTATTGGTCAGCCCGCTTCCA | 519 |
| | | TATCATGTAAATCATCACAATCTCTTCTTGATAGCGATGGCAAGACTTATTTGAACTGGTT | |
| | | GTTGCAACGCCCAGGTCAGAGCCCTAAGAGACTTATCTATTTGGTGAGCAAACTCGACAGC | |
| | | GGTGCACCCGATCGTTTTACCGGAAGCGGCAGCGGCACCGATTTCACACTGAAGATCAGTA | |
| | | GGGTCGAAGCTGAAGACCTGGGAATCTACTACTGCTGGCAAGGTACTCACTTTCCCCTGAC | |
| | | TTTCGGCTGCGGTACTAAACTTGAGATCAAA | |
| GPB03 | VH1 | CAGGTGCAACTCGTTCAAAGCGGGGCCGAGGTGAAGAAACCAGGGGCCTCAGTTAAGGTGA | 434 |
| | | GTTGCAAGGCAAGTGGATACACTTTCACCGATTATGAAATGCATTGGGTGCGTCAGGCCCC | |
| | | AGGACAAGGACTGGAGTGGATGGGCGCTCTCGATCCTAAGACTGGTGATACTGCTTACTCT | |
| | | CAAAAGTTCAAAGGCCGAGTCACCTTGACCGCCGACAAGTCCACATCCACTGCATATATGG | |
| | | AATTGTCAAGTCTGACAAGCGAAGATACAGCCGTCTACTACTGCACCCGCTTTTATAGCTA | |
| | | TACATATTGGGGACAGGGGACCTTGGTTACTGTGTCATCT | |
| | VH2 | GAAGTGCAACTGGTCGAAACAGGCGGGGGACTGGTACAGCCCAAGGGATCTTTGAAACTTA | 522 |
| | | GTTGTGCTGCTAGTGGGTTTACATTCAATGCCTCCGCAATGAACTGGGTAAGACAAGCTCC | |
| | | TGGCAAGTGCCTGGAATGGGTGGCCCGTATTCGCTCTAAAAGTAATAACTACGCTATTTAT | |
| | | TACGCTGATTCTGTAAAGGATCGGTTTACAATAAGTCGGGACGACAGCCAATCCATGCTGT | |
| | | ATCTCCAAATGAATAACCTGAAAACAGAGGATACTGCCATGTACTATTGTGTGCGGGACCC | |
| | | AGGCTATTACGGGAATCCCTGGTTCGCCTATTGGGGACAGGGCACTCTGGTTACCGTATCA | |
| | | TCA | |
| | VL1 | GACGTGGTAATGACACAATCACCTTTGTCTCTTCCCGTAACCCCCGGTGAACCAGCCAGCA | 435 |
| | | TCTCATGCAGAAGCAGTCAGTCACTGGTACATTCCAACCGTAATACTTATCTTCACTGGTA | |
| | | CTTGCAGAAGCCTGGGCAGTCTCCTCAACTTTTGATATATAAAGTGAGCAATCGGTTTAGC | |
| | | GGTGTCCCAGACCGCTTTTCTGGATCTGGAAGTGGAACAGACTTTACTCTGAAAATAAGCA | |
| | | GAGTCGAGGCAGAAGATGTCGGAGTTTACTACTGTAGCCAGAACACACACGTACCCCCAAC | |
| | | CTTTGGACAGGGCACAAAGTTGGAAATCAAG | |
| | VL2 | CAAATTGTCCTCACCCAATCACCAGCTATAATGTCTGCTTTTCCCGGCGAGAAAGTAACCA | 523 |
| | | TGACTTGTAGCGCCTCTAGTAGCGTGTCATATATGTATTGGTATCAACAAAAGAGCGGTAG | |
| | | TTCACCTCGACTCCTTATCTACGACACAAGTAATCTCGCTAGTGGTGTCCCAGTCCGTTTC | |
| | | TCCGGGAGCGGCAGCGGCACATCATACTCCCTGACCATCTCCAGAATGGAGGCCGAGGACG | |
| | | CTGCCACATACTATTGTCAGCAGTGGAGCTCATATCCTTTGACATTCGGTTGCGGTACTAA | |
| | | ACTCGAAATCAAG | |
| GPB04 | VH1 | GAAGTTCAGCTTGTCGAAAGTGGCGGAGGTCTTGTAAAGCCAGGAGGGTCCCTGAAGTTGA | 524 |
| | | GTTGTGCTGCCTCTGGCTTCACCTTTTCACGGTATGCCATGTCCTGGGTTCGACAGATACC | |
| | | TGAGAAGATTCTTGAATGGGTGGCCGCAATAGACAGTAGCGGTGGTGACACCTATTACCTC | |
| | | GACACAGTCAAAGACCGCTTTACTATCTCACGCGATAACGCCAACAATACCCTGCACTTGC | |
| | | AGATGCGATCACTTCGTTCAGAAGACACTGCTCTTTACTATTGTGTACGCCAAGGGGGAGC | |
| | | ATACTGGGGTCAGGGAACACTGGTTACCGTGTCTTCA | |
| | VH2 | CAGGTTCAGCTTGTGCAATCAGGCGCTGAAGTCAAAAAACCAGGAGCAAGCGTGAAAGTAA | 520 |
| | | GCTGTAAGGCTTCCGGTTACACTTTCACCGATTACGAAATGCACTGGGTACGCCAGGCTCC | |
| | | TGGACAATGTCTGGAATGGATGGGCGCACTTGATCCAAAAACAGGTGACACTGCTTACAGT | |
| | | CAAAAATTCAAAGGTCGTGTCACTCTTACAGCAGATAAGTCCACCAGTACCGCTTATATGG | |
| | | AGCTTAGCTCCCTGACTTCCGAGGACACCGCCGTGTATTATTGTACAAGATTCTACTCATA | |
| | | TACTTACTGGGGCCAAGGAACCCTGGTGACAGTGTCATCT | |
| | VL1 | GACGTGGTTATGACTCAGACTCCTCTGACTCTCTCCGTCACTATTGGTCAGCCCGCTTCCA | 525 |
| | | TATCATGTAAATCATCACAATCTCTTCTTGATAGCGATGGCAAGACTTATTTGAACTGGTT | |
| | | GTTGCAACGCCCAGGTCAGAGCCCTAAGAGACTTATCTATTTGGTGAGCAAACTCGACAGC | |
| | | GGTGCACCCGATCGTTTTACCGGAAGCGGCAGCGGCACCGATTTCACACTGAAGATCAGTA | |
| | | GGGTCGAAGCTGAAGACCTGGGAATCTACTACTGCTGGCAAGGTACTCACTTTCCCCTGAC | |
| | | TTTCGGCGCCGGTACTAAACTTGAGATCAAA | |
| | VL2 | GATGTTGTTATGACTCAGTCACCTCTCTCACTTCCTGTAACCCCTGGTGAACCAGCCTCTA | 521 |
| | | TAAGCTGCCGGTCAAGTCAAAGCCTGGTTCATAGCAACCGTAACACTTACCTTCACTGGTA | |
| | | CTTGCAAAAACCTGGTCAGTCCCCACAACTCTTGATCTACAAAGTCTCCAATCGCTTCTCT | |
| | | GGAGTCCCTGACAGGTTTTCTGGTAGTGGATCAGGTACAGACTTCACTTTGAAAATCAGCC | |
| | | GCGTTGAGGCCGAGGACGTGGGAGTGTATTATTGCTCTCAGAATACCCATGTACCCCCAAC | |
| | | CTTTGGCTGTGGGACTAAACTCGAGATTAAA | |
| GPB06 | VH1 | GAAGTGCAACTGGTCGAAACAGGCGGGGGACTGGTACAGCCCAAGGGATCTTTGAAACTTA | 526 |
| | | GTTGTGCTGCTAGTGGGTTTACATTCAATGCCTCCGCAATGAACTGGGTAAGACAAGCTCC | |
| | | TGGCAAGGGCCTGGAATGGGTGGCCCGTATTCGCTCTAAAAGTAATAACTACGCTATTTAT | |
| | | TACGCTGATTCTGTAAAGGATCGGTTTACAATAAGTCGGGACGACAGCCAATCCATGCTGT | |
| | | ATCTCCAAATGAATAACCTGAAAACAGAGGATACTGCCATGTACTATTGTGTGCGGGACCC | |
| | | AGGCTATTACGGGAATCCCTGGTTCGCCTATTGGGGACAGGGCACTCTGGTTACCGTATCA | |
| | | TCA | |
| | VH2 | CAGGTTCAGCTTGTGCAATCAGGCGCTGAAGTCAAAAAACCAGGAGCAAGCGTGAAAGTAA | 520 |
| | | GCTGTAAGGCTTCCGGTTACACTTTCACCGATTACGAAATGCACTGGGTACGCCAGGCTCC | |
| | | TGGACAATGTCTGGAATGGATGGGCGCACTTGATCCAAAAACAGGTGACACTGCTTACAGT | |
| | | CAAAAATTCAAAGGTCGTGTCACTCTTACAGCAGATAAGTCCACCAGTACCGCTTATATGG | |
| | | AGCTTAGCTCCCTGACTTCCGAGGACACCGCCGTGTATTATTGTACAAGATTCTACTCATA | |
| | | TACTTACTGGGGCCAAGGAACCCTGGTGACAGTGTCATCT | |
| | VL1 | CAAATTGTCCTCACCCAATCACCAGCTATAATGTCTGCTTTTCCCGGCGAGAAAGTAACCA | 527 |
| | | TGACTTGTAGCGCCTCTAGTAGCGTGTCATATATGTATTGGTATCAACAAAAGAGCGGTAG | |
| | | TTCACCTCGACTCCTTATCTACGACACAAGTAATCTCGCTAGTGGTGTCCCAGTCCGTTTC | |
| | | TCCGGGAGCGGCAGCGGCACATCATACTCCCTGACCATCTCCAGAATGGAGGCCGAGGACG | |
| | | CTGCCACATACTATTGTCAGCAGTGGAGCTCATATCCTTTGACATTCGGTGGAGGTACTCC | |
| | | ACTCGAAATCAAG | |
| | VL2 | GATGTTGTTATGACTCAGTCACCTCTCTCACTTCCTGTAACCCCTGGTGAACCAGCCTCTA | 521 |
| | | TAAGCTGCCGGTCAAGTCAAAGCCTGGTTCATAGCAACCGTAACACTTACCTTCACTGGTA | |
| | | CTTGCAAAAACCTGGTCAGTCCCCACAACTCTTGATCTACAAAGTCTCCAATCGCTTCTCT | |
| | | GGAGTCCCTGACAGGTTTTCTGGTAGTGGATCAGGTACAGACTTCACTTTGAAAATCAGCC | |
| | | GCGTTGAGGCCGAGGACGTGGGAGTGTATTATTGCTCTCAGAATACCCATGTACCCCCAAC | |
| | | CTTTGGCTGTGGGACTAAACTCGAGATTAAA | |

Table 48 below shows the heavy chain and light chain CDR sequences of the engineered antibodies targeting GPC-3.

TABLE 48

| Name | CDR | Sequence | SEQ ID NO |
|---|---|---|---|
| GPM01 | CDR-H1 | RYAMS | 93 |
| | CDR-H2 | AIDSSGGDTYYLDTVKD | 94 |
| | CDR-H3 | QGGAY | 95 |
| | CDR-L1 | KSSQSLLDSDGKTYLN | 96 |
| | CDR-L2 | LVSKLDS | 97 |
| | CDR-L3 | WQGTHFPLT | 98 |
| GPM02 | CDR-H1 | DYEMH | 99 |
| | CDR-H2 | ALDPKTGDTAYSQKFKG | 100 |
| | CDR-H3 | FYSYTY | 101 |
| | CDR-L1 | RSSQSLVHSNRNTYLH | 102 |
| | CDR-L2 | KVSNRFS | 103 |
| | CDR-L3 | SQNTHYPPT | 104 |
| GPM04 | CDR-H1 | ASAMN | 105 |
| | CDR-H2 | RIRSKSNNYAIYYAADSVKD | 106 |
| | CDR-H3 | DPGYYGNPWFAY | 107 |
| | CDR-L1 | SASSSYSWY | 108 |
| | CDR-L2 | DTSNLAS | 109 |
| | CDR-L3 | QQWSSYPLT | 110 |
| GPB01 | V1 CDR-H1 | DYEMH | 99 |
| | V1 CDR-H2 | ALDPKTGDTAYSQKFKG | 100 |
| | V1 CDR-H3 | FYSYTY | 101 |
| | V1 CDR-L1 | RSSQSLVHSNRNTYLH | 102 |
| | V1 CDR-L2 | KVSNRFS | 103 |
| | V1 CDR-L3 | SQNTHVPPT | 104 |
| | V2 CDR-H1 | RYAMS | 93 |
| | V2 CDR-H2 | AIDSSGGDTYYLDTVKD | 94 |
| | V2 CDR-H3 | QGGQY | 95 |
| | V2 CDR-L1 | KSSQSLLDSDGKTYLN | 96 |
| | V2 CDR-L2 | LVSKLDS | 97 |
| | V2 CDR-L3 | WQGTHFPLT | 98 |
| GPB03 | V1 CDR-H1 | DYEMH | 99 |
| | V1 CDR-H2 | ALDPKTGDTAYSQKFKG | 100 |
| | V1 CDR-H3 | FYSYTY | 101 |
| | V1 CDR-L1 | RSSQSLVHSNRNTYLH | 102 |
| | V1 CDR-L2 | KVSNRFS | 103 |
| | V1 CDR-L3 | SQNTHVPPT | 104 |
| | V2 CDR-H1 | ASAMN | 105 |
| | V2 CDR-H2 | RIRSKSNNYAIYYADSVKD | 106 |
| | V2 CDR-H3 | DPGYYGNPWFAY | 107 |
| | V2 CDR-L1 | SASSSVSYMY | 108 |
| | V2 CDR-L2 | DTSNLAS | 100 |
| | V2 CDR-L3 | QQWSSYPLT | 110 |
| GPB04 | V1 CDR-H1 | RYAMS | 93 |
| | V1 CDR-H2 | AIDSSGGDTYYLDTVKD | 94 |
| | V1 CDR-H3 | QGGAY | 95 |
| | V1 CDR-L1 | KSSQSLLDSDGKTYLN | 96 |
| | V1 CDR-L2 | LVSKLDS | 97 |
| | V1 CDR-L3 | WQGTHFPLT | 98 |
| | V2 CDR-H1 | DYEMH | 99 |
| | V2 CDR-H2 | ALDPKTGDTAYSQKFKG | 100 |
| | V2 CDR-H3 | FYSYTY | 101 |
| | V2 CDR-L1 | RSSQSLVHSNRNTYLH | 102 |
| | V2 CDR-L2 | KVSNRFS | 103 |
| | V2 CDR-L3 | SQNTHVPPT | 104 |
| GPB06 | V1 CDR-H1 | ASAMN | 105 |
| | V1 CDR-H2 | RIRSKSMMYAIIADSVKD | 106 |
| | V1 CDR-H3 | DPGYYGNPWFAY | 107 |
| | V1 CDR-L1 | SASSSVSYMY | 108 |
| | V1 CDR-L2 | DTSNLAS | 100 |
| | V1 CDR-L3 | QQWSSYPLT | 110 |
| | V2 CDR-H1 | DYENH | 99 |
| | V2 CDR-H2 | ALDFKTGDTAYSQKFKG | 100 |
| | V2 CDR-H3 | FYSYTY | 101 |
| | V2 CDR-L1 | RSSQSLVHSNRNTYLH | 102 |
| | V2 CDR-L2 | KVSNRFS | 103 |
| | V2 CDR-L3 | SQNTHVPPT | 104 |

The GPC-3 protein binding constants of GPM01, GPM02, GPM04, GPB01, GPB03, GPB04, and GPB06 were determined using the Octet Red96e (Sartorius). In order to analyze the binding constants of the seven antibodies, the human GPC-3 recombinant protein (Sino Biologicals, 10088-H08H) was loaded onto the Anti-Penta-HIS (HIS1K) biosensor (Sartorius, 18-5120). Then the seven antibodies were added in a binding reaction (300 seconds) and a dissociation reaction (1,200 seconds) at various concentrations, and their affinities for GPC-3 were calculated (FIG. 42, Table 49). Table 49 below illustrates the binding constants of the engineered antibodies targeting GPC-3.

TABLE 49

| Antibodies | Antigen | Binding mode | K_{D} (nM) | Kₐ (1/Ms) | K_{d} (1/s) |
|---|---|---|---|---|---|
| GPM01 | rhGPC3 | Monovalent | 2.7048 | 3.72E+05 | 1.01E−03 |
| GPM02 | rhGPC3 | Monovalent | 2.3799 | 1.92E+05 | 4.57E−04 |
| GPM04 | rhGPC3 | Monovalent | 38.3721 | 2.92E+04 | 1.12E−03 |
| GPB01 | rhGPC3 | Biparatopic | 0.5827 | 2.49E+05 | 1.45E−04 |
| GPB03 | rhGPC3 | Biparatopic | 0.1601 | 2.03E+05 | 3.25E−05 |
| GPB04 | rhGPC3 | Biparatopic | 0.2510 | 3.32E+05 | 8.33E−05 |
| GPB06 | rhGPC3 | Biparatopic | 0.3427 | 6.64E+04 | 2.28E−05 |

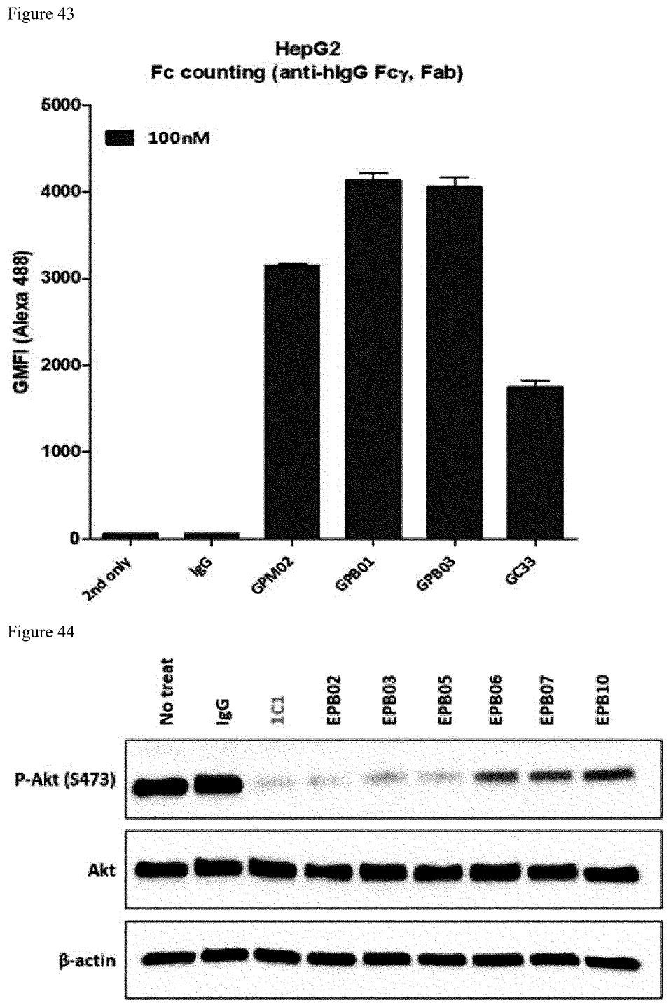

HepG2 liver cancer cell line was used to quantify the Fc loads on the surface of GPC-3 expressing cells. 100 nM human IgG1, GPM02, GPB01, GPB03, and GC33 were allowed to bind to the HepG2 cell line at 4° C. for 30 minutes, and the Fc loads were quantified using the Alexa 488 fluorescence-conjugated anti-human IgG Fcγ Fab antibody (Jackson ImmunoResearch, 109-547-008) (FIG. 43). GC33, which is a humanized antibody targeting GPC-3, was used as a positive control (Nakano et al., U.S. Pat. No. 7,919,086 B2). It was shown that higher Fc loads on the surface of cancer cells are induced by treatment of GPM02, GPB01, and GPB03 compared to GC33 (FIG. 43).

### Example 18. Design, Preparation and Analysis of Antibody Structure Targeting EPH Receptor A2 (EphA2)

The variant light chain and heavy chain polypeptide sequences of the antibodies that specifically bind to the EPH receptor A2 (EphA2) protein are shown in Table 46. For EPB01, expression vectors consisting of the sequences corresponding to Fc-Hole (SEQ ID NO: 7), EPB01 HC (SEQ ID NO: 111), and EPB01 LC (SEQ ID NO: 112) were co-transfected into EXPICHO-S™ (Gibco, A29127) (FIG. 41b, Table 50), and purification and analysis were performed in the same manner as described in Example 1. Expression, purification and analysis were performed for EPB02, EPB03, EPB04, EPB05, EPB06, EPB07, EPB08, EPB09, EPB10, EPB11, and EPB12 in the same manner as mentioned above (Table 50). The 12 antibodies have structures in which the variable regions that bind to two different epitopes of EphA2 are linked with a polypeptide linker (SEQ ID NO: 48, SEQ ID NO: 50), bind biparatopically to EphA2, and have two Fc domains (FIG. 41b, Table 50).

TABLE 50

| | | SEQ |
|---|---|---|
| | | ID |
| Name | Sequence | NO |
| EPB01 HC | EVQLLESGGGLVQPGGSLRLSCAASGFTFSRYQMMWVRQAPGKGLEWVSSISPSGGVTL | 111 |
| | YADSVKGRFTISRDNSKNTLYLQMNSLRAEDTAVYYCTRELLGTVVVPVAWKMRGYFDY | |
| | WGQLTLVTVSSASTKGPSVFPLAPEVQLLESGGGLVQPGGSLRLSCAASGFTFSHYMMA | |
| | WVRQAPGKCLEWVSRIGPSGGPTHYADSVKGRFTISRDNSKNTLYLQMNSLRAEDTAVY | |
| | YCAGYDSGYDYVAVAGPAEYFQHWGQGTLVTVSSASTKGPSVFPLAPSSKSTSGGTAAL | |
| | GCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSLVTVLHQDWLNGKEY | |
| | KCKVSNKALPAPIEKTISKAKGQPREPQVYTLPPCREEMTKNQVSLWCLVKGFYPSDIA | |
| | VEWESNGQPENNYKTTPPVLDSDGSFFLYSKLTVDKSRWQQGNVFSCSVMHEALHNHYT | |
| | QKSLSLSPGK | |
| EPB01 LC | DIQMTQSPGTLSVSPGERATLSCRASQSVSSNLAWYQQKPGQAPRLLIYGASTRATGIP | 112 |
| | ARFSGSGSGTEFTLTISSMQSEDFAVYYCQQYNNWPPLTFGGGTKVEIKRTVAAPSVFI | |
| | FPPDIQMTQSPSSLSASVGDRVTITCRASQSISTWLAWYQQKPGKAPKLLIYKASNLHT | |
| | GVPSRFSGSGSGTEFSLTISGLQPDDFATYYCQQYNSYSRTFGCGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPLPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB02 HC | EVQLVESGGGVVRPGGSLRLSCAASGFTVSDYSMNWVRQAPGKGLEWIGFIRNKANAYT | 113 |
| | TEYSASVKGRFTISRDDSKNTLYLQMNSLKTEDTAVYYCTTYPRYHAMDSWGQGTMVTV | |
| | SSASTKGPSVFPLAPEVQLLESGGGLVQPGGSLRLSCAASGFTFSHYMMAWVRQAPGKC | |
| | LEWVSRIGPSGGPTHYADSVKGRFTISRDNSKNTLYLQMNSLRAEDTAVYYCAGYDSGY | |
| | DYVAVAGPAEYFQHWGQGTLVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNHKPSNTK | |
| | VDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPSDIAVEWESNGQP | |
| | ENNYKTTPPVLDSDGSFFLYSKLTVDKSRWQQGNVFSCSVMHEALHNHYTQKSLSLSPG | |
| | K | |
| EPB02 LC | AIQLTQSPSSLSASVGDRVTITCRASQSISNNLHWYLQKPGQSPSLLIYYGFQSISGVP | 114 |
| | SRFSGSGSGTDFTLTISSLQPEDFATYYCQQANSWPLTFGGGTKLEIKRTVAAPSVFIF | |
| | FPDIQMTQSPSSLSASVGDRVTITCRASQSISTWLAWYQQKPGKAPKLLIYKASNLHTG | |
| | VPSRFSGSGSGTEFSLTISGLQPDDFATYYCQQYNSYSRTFGCGTKVEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| EPB03 HC | QVQLLESGGGLVQPGGSLRLSCAASGFTFSSYTMSWVRQAPGQCLEWMGTISSGGTYTY | 115 |
| | YPDSVKGRFTISRDNAKNSLYLQMNSLRAEDTAVYYCAREAIFTYWGRGTLVTVSSAST | |
| | KGPSVFPLAPEVQLLESGGGLVQPGGSLRLSCAASGFTFSHYMMAWVRQAPGKCLEWVS | |
| | RIGPSGGPTHYADSVKGRFTISRDNSKNTLYLQMNSLRAEDTAVYYCAGYDSGYDYVAV | |
| | AGPAEYFQHWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQKSLSLSPGK | |
| EPB03 LC | DIQMTQSPGTLSVSPGERVTLSCKASQDINNYLSWYQQKPDQAPKLLIKRANRLVDGVP | 116 |
| | DRFSGSGSGTDFTLKISRVEAEDVGVYYCLKYDEFPYTFGQGTRLEIKRTVAAPSVFIF | |
| | PPDIQMTQSPSSLSASVGDRVTITCRASQSISTWLAWYQQKPGKAPKLLIYKASNLHTG | |
| | VPSRFSGSGSGTEFSLTISGLQPDDFATYYCQQYNSYSRTFGCGTKVEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| EPB04 HC | EVQLLESGGGLVQPGGSLRLSCAASGFTFSHYMMAWVRQAPGKCLEWVSRIGPSGGPTH | 117 |
| | YADSVKGRFTISRDNSKNTLYLQMNSLRAEDTAVYYCAGYDSGYDYVAVAGPAEYFQHW | |
| | GQGTLVTVSSASTKGPSVFPLAPEVQLLESGGGLVQPGGSLRLSCAASGFTFSRYQMMW | |
| | VRQAPGKCLEWVSSISPSGGVTLYADSVKGRFTISRDNSKNTLYLQMNSLRAEDTAVYY | |
| | CTRELLGTVVVPVAWKMRGYFDYWGQLTLVTVSSASTKGPSVFPLAPSSKSTSGGTAAL | |
| | GCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLWCLVKGFYPSDIA | |
| | VEWESNGQPENNYKTTPPVLDSDGSFFLYSKLTVDKSRWQQGNVFSCSVMHEALHNHYT | |
| | QKSLSLSPGK | |
| EPB04 LC | DIQMTQSPGTLSVSPGERVTLSCKASQDINNYLSWYQQKPDQAPKLLIKRANRLVDGVP | 118 |
| | SRFSGSGSGTEFSLTISGLQPDDFATYYCQQYNSYSRTFGQGTRLEIKRTVAAPSVFIF | |
| | PPDIQMTQSPGTLSVSPGERATLSCRASQSVSSNLAWYQQKPGKAPKLLIYKASNLHTG | |
| | IPARFSGSGSGTEFSLTISSMQSEDFAVYYCQQYNNWPPLTFGGGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPLPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB05 HC | EVQLLESGGGLVQPGGSLRLSCAASGFTFSHYMMAWVRQAPGKGLEWVSRIGPSGGPTH | 119 |
| | YADSVKGRFTISRDNSKNTLYLQMNSLRAEDTAVYYCAGYDSGYDYVAVAGPAEYFQHW | |
| | GQGTLVTVSSASTKGPSVFPLAPEVQLLESGGGVVRPGGSLRLSCAASGFTFSRYSMMW | |
| | VRQAPGKCLEWIGFIRNKANAYTTEYSASVKGRFTISRDDSKNTLYLQMNSLKTEDTAV | |
| | YYCTTYPRYHAMDSWGQGTMVTVSSASTKGPSVFPLAPSSKSTSGGTAALGCLVKDYFP | |
| | EPVTVSWNSGALTSGVHTFPAVLQSSGLYSLSSVVTVPSSSLGTQTYICNVNHKPSNTK | |
| | VDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKAL | |
| | PAPIEKTISKAKGQPREPQVYTLPPCREEMTKNQVSLWCLVKGFYPSDIAVEWESNGQP | |
| | ENNYKTTPPVLDSDGSFFLYSKLTVDKSRWQQGNVFSCSVMHEALHNHYTQKSLSLSPG | |
| | K | |
| EPB05 LC | DIQMTQSPGTLSVSPGERVTLSCKASQDINNYLSWYQQKPDQAPKLLIKRANRLVDGVP | 120 |
| | SRFSGSGSGTEFSLTISGLQPDDFATYYCQQYNSYSRTFGQGTKVEIKRTVAAPSVFIF | |
| | PPAIQLTQSPSSLSASVGDRVTITCRASQSISNNLHWYLQKPGQSPSLLIYYGFQSISG | |
| | VPSRFSGSGSGTDFTLTISSLQPEDFATYYCQQANSWPLTFGCGTKLEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPLPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| EPB06 LC | EVQLLESGGGLVQPGGSLRLSCAASGFTFSHYMMAWVRQAPGKGLEWVSRIGPSGGPTH | 121 |
| | YADSVKGRFTISRDNSKNTLYLQMNSLRAEDTAVYYCAGYDSGYDYVAVAGPAEYFQHW | |
| | GQGTLVTVSSASTKGPSVFPLAPQVQLLESGGGLVQPGGSLRLSCAASGFTFSRYTMSW | |
| | VRQAPGQCLEWMGTISSGGTYTYYPDSVKGRFTISRDNAKNSLYLQMNSLRAEDTAVYY | |
| | CAREAIFTYWGRGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTKVDKKV | |
| | EPKSCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPCREEMTKNQVSLWCLVKGFYPSDIAVEWESNGQPENNYK | |
| | TTPPVLDSDGSFFLYSKLTVDKSRWQQGNVFSCSVMHEALHNHYTQKSLSLSPGK | |
| EPB06 HC | DIQMTQSPSSLSASVGDRVTITCRASQSISTWLAWYQQKPDQAPKLLIKRANRLVDGVP | 122 |
| | SRFSGSGSGTEFSLTISGLQPDDFATYYCQQYNSYSRTFGQGTKVEIKRTVAAPSVFIF | |
| | PPDIQLTQSPSSLSLSPGERVTLSCKASQDINNYLSWYQQKPDQAPKLLIKRANRLVDG | |
| | VPSRFSGSGSGTDFTLTISSLQPEDFATYYCQQANSWPLTFGCGTKLEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPLPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| EPB07 HC | EVQLVESGGGVVRPGGSLRLSCAASGFTVSDYSMNWVRQAPGKGLEWIGFIRNKANAYT | 123 |
| | TEYSASVKGRFTISRDDSKNTLYLQMNSLKTEDTAVYYCTTYPRYHAMDSWGQGTMVTV | |
| | SSASTKGPSVFPLAPEVQLLESGGGLVQPGGSLRLSCAASGFTFSRYQMMWVRQAPGKC | |
| | LEWVSSISPSGGVTLYADSVKGRFTISRDNSKNTLYLQMNSLRAEDTAVYYCAGYDSGY | |
| | VVVPVAWKMRGYFDYWGQLTLVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVVVDVSH | |
| | EDPEVKFNWYVDGVEVHNAKTKPREEQYNSTYRVVSVLTVLHQDWLNGKEYKCKVSNKA | |
| | LPAPIEKTISKAKGQPREPQVYTLPPCREEMTKNQVSLWCLVKGFYPSDIAVEWESNGQ | |
| | PENNYKTTPPVLDSDGSFFLYSKLTVDKSRWQQGNVFSCSVMHEALHNHYTQKSLSLSP | |
| | GK | |
| EPB07 LC | AIQLTQSPSSLSASVGDRVTITCRASQSISNNLHWYLQKPGQSPSLLIYYGFQSISGVP | 124 |
| | SRFSGSGSGTDFTLTISSLQPEDFATYYCQQANSWPLTFGGGTKLEIKRTVAAPSVFIF | |
| | PPDIQMTQSPGTLSVSPGERVTLSCKASQDINNYLSWYQQKPGQAPRLLIYGASTRATG | |
| | IPARFSGSGSGTEFTLTISSMQSEDFAVYYCQQYNNWPPLTFGCGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB08 HC | QVQLLESGGGLVQPGGSLRLSCAASGFTFSSYTMSWVRQAPGQCLEWMGTISSGGTYTY | 125 |
| | YPDSVKGRFTISRDNAKNSLYLQMNSLRAEDTAVYYCAREAIFTYWGRGTLVTVSSAST | |
| | KGPSVFPLAPEVQLLESGGGLVQPGGSLRLSCAASGFTFSHYMMAWVRQAPGKCLEWVS | |
| | SISPSGGVTLYADSVKGRFTISRDNSKNTLYLQMNSLRAEDTAVYYCTRELLGTVVVPV | |
| | AWKMRGYFDYWGQLTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALPAPI | |
| | EKTISKAKGQPREPQVYTLPPCREEMTKNQVSLWCLVKGFYPSDIAVEWESNGQPENNY | |
| | KTTPPVLDSDGSFFLYSKLTVDKSRWQQGNVFSCSVMHEALHNHYTQKSLSLSPGK | |
| EPB08 LC | DIQMTQSPGTLSVSPGERVTLSCKASQDINNYLSWYQQKPDQAPKLLIKRANRLVDGVP | 126 |
| | DRFSGSGSGTDFTLKISRVEAEDVGVYYCLKYDEFPYTFGQGTRLEIKRTVAAPSVFIF | |
| | PPDIQMTQSPGTLSVSPGERATLSCRASQSVSSNLAWYQQKPGKAPKLLIYGASTRATG | |
| | IPARFSGSGSGTEFTLTISSMQSEDFAVYYCQQYNNWPPLTFGCGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB09 HC | EVQLLESGGGLVQPGGSLRLSCAASGFTFSRYQMMWVRQAPGKGLEWVSSISPSGGVTL | 127 |
| | YADSVKGRFTISRDNSKNTLYLQMNSLRAEDTAVYYCTRELLGTVVVPVAWKMRGYFDY | |
| | WGQLTLVTVSSASTKGPSVFPLAPEVQLVESGGGVVRPGGSLRLSCAASGFTVSDYSMN | |
| | WVRQAPGKCLEWIGFIRNKANAYTTEYSASVKGRFTISRDDSKNTLYLQMNSLKTEDTA | |
| | VYYCTTYPRYHAMDSWGQGTMVTVSSASTKGPSVFPLAPSSKSTSGGTAALGCLVKDYF | |
| | PEPVTVSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVVVDVSH | |
| | EDPEVKFNWYVDGVEVHNAKTKPREEQYNSTYRVVSLVTVLHQDWLNGKEYKCKVSNKA | |
| | LPAPIEKTISKAKGQPREPQVYTLPPCREEMTKNQVSLWCLVKGFYPSDIAVEWESNGQ | |
| | PENNYKTTPPVLDSDGSFFLYSKLTVDKSRWQQGNVFSCSVMHEALHNHYTQKSLSLSP | |
| | GK | |
| EPB09 LC | DIQMTQSPGTLSVSPGERATLSCRASQSVSSNLAWYQQKPGQAPRLLIYGASTRATGIP | 128 |
| | ARFSGSGSGTEFTLTISSMQSEDFAVYYCQQYNNWPPLTFGGGTKVEIKRTVAAPSVFI | |
| | FPPDIQLTQSPSSLSASVGDRVTITCRASQSISNNLHWYLQKPGQSPSLLIYYGFQSIS | |
| | GVPSRFSGSGSGTDFSLTISSLQPEDFATYYCQQANSWPLTFGCGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPLPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB10 HC | QVQLLESGGGLVQPGGSLRLSCAASGFTFSSYTMSWVRQAPGQCLEWMGTISSGGTYTY | 129 |
| | YPDSVKGRFTISRDNAKNSLYLQMNSLRAEDTAVYYCAREAIFTYWGRGTLVTVSSAST | |
| | KGPSVFPLAPEVQLVESGGGVVRPGGSLRLSCAASGFTVSDYSMNWVRQAPGKGLEWIG | |
| | FIRNKANAYTTEYSASVKGRFTISRDDSKNTLYLQMNSLKTEDTAVYYCTTYPRYHAMD | |
| | SWGQGTMVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTISKAKG | |
| | QPREPQVYTLPPCREEMTKNQVSLWCLVKGFYPSDIAVEWESNGQPENNYKTTPPVLDS | |
| | DGSFFLYSKLTVDKSRWQQGNVFSCSVMHEALHNHYTQKSLSLSPGK | |
| EPB10 LC | DIQLTQSPSSLSLSPGERVTLSCKASQDINNYLSWYQQKPDQAPKLLIKRANRLVDGVP | 130 |
| | DRFSGSGSGTDFTLKISRVEAEDVGVYYCLKYDEFPYTFGQGTRLEIKRTVAAPSVFIF | |
| | PPAIQLTQSPSSLSASVGDRVTITCRASQSISNNLHWYLQKPGQSPSLLIYYGFQSISG | |
| | VPSRFSGSGSGTDFTLTISSLQPEDFATYYCQQANSWPLTFGCGTKLEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPLPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |
| EPB11 HC | EVQLLESGGGLVQPGGSLRLSCAASGFTFSRYQMMWVRQAPGKGLEWVSSISPSGGVTL | 131 |
| | YADSVKGRFTISRDNSKNTLYLQMNSLRAEDTAVYYCTRELLGTVVVPVAWKMRGYFDY | |
| | WGQGTMVTVSSASTKGPSVFPLAPEVQLLESGGGLVQPGGSLRLSCAASGFTFSSYTMS | |
| | WVRQAPGQCLEWMGTISSGGTYTYYPDSVKGRFTISRDNAKNSLYLQMNSLRAEDTAVY | |
| | YCAREAIFTYWGRGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNTKVDKK | |
| | VEPKSCDKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALPAPI | |
| | EKTISKAKGQPREPQVYTLPPCREEMTKNQVSLWCLVKGFYPSDIAVEWESNGQPENNY | |
| | KTTPPVLDSDGSFFLYSKLTVDKSRWQQGNVFSCSVMHEALHNHYTQKSLSLSPGK | |
| EPB11 LC | DIQMTQSPGTLSVSPGERATLSCRASQSVSSNLAWYQQKPGQAPRLLIYGASTRATGIP | 132 |
| | ARFSGSGSGTEFTLTISSMQSEDFAVYYCQQYNNWPPLTFGGGTKVEIKRTVAAPSVFI | |
| | FPPDIQLTQSPSSLSLSPGERVTLSCKASQDINNYLSWYQQKPDQAPKLLIKRANRLVD | |
| | GVPSRFSGSGSGTEFSLTISGLQPDDFATYYCQQYNSYSRTFGCGTKVEIKRTVAAPSV | |
| | FIFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGECGGGGSGGGGSGGGGSEPKS | |
| | SDKTHTCPPCPAPELLGGPSVFLFPPLPKDTLMISRTPEVTCVVVDVSHEDPEVKFNWY | |
| | VDGVEVHNAKTKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNGQPENNYKTTPP | |
| | VLDSDGSFFLYSKLTVDKSRWQQGNVFSCSVMHEALHNHYTQKSLSLSPGK | |
| EPB12 HC | EVQLVESGGGVVRPGGSLRLSCAASGFTVSDYSMNWVRQAPGKGLEWIGFIRNKANAYT | 133 |
| | TEYSASVKGRFTISRDDSKNTLYLQMNSLKTEDTAVYYCTTYPRYHAMDSWGQGTMVTV | |
| | SSASTKGPSVFPLAPEVQLLESGGGLVQPGGSLRLSCAASGFTFSSYTMSWVRQAPGQC | |
| | LEWMGTISSGGTYTYYPDSVKGRFTISRDNAKNSLYLQMNSLRAEDTAVYYCAREAIFT | |
| | YWGRGTLVTVSSASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTISKAKG | |
| | QPREPQVYTLPPCREEMTKNQVSLWCLVKGFYPSDIAVEWESNGQPENNYKTTPPVLDS | |
| | DGSFFLYSKLTVDKSRWQQGNVFSCSVMHEALHNHYTQKSLSLSPGK | |
| EPB12 LC | AIQLTQSPSSLSASVGDRVTITCRASQSISNNLHWYLQKPGQSPSLLIYYGFQSISGVP | 134 |
| | SRFSGSGSGTDFTLTISSLQPEDFATYYCQQANSWPLTFGGGTKLEIKRTVAAPSVFIF | |
| | FPDIQMTQSPSSLSASVGDRVTITCRASQSISTWLAWYQQKPGKAPKLLIYKASNLHTG | |
| | VPSRFSGSGSGTEFSLTISGLQPDDFATYYCQQYNSYSRTFGCGTKVEIKRTVAAPSVF | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGECGGGGSGGGGSGGGGSEPKSS | |
| | DKTHTCPPCPAPELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKALPAPIEKTISK | |
| | AKGQPREPQVYTLPPCREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPGK | |

TABLE 51

| Name | Sequence | SEQ ID NO |
|---|---|---|
| EPB01 HC | GAGGTGCAACTGCTTGAGTCAGGAGGAGGACTGGTTCAACCCGGCGGGAGCCTTCGACT | 528 |
| | CTCATGTGCAGCCTCTGGGTTTACATTTAGTCGTTACCAAATGATGTGGGTACGCCAGG | |
| | CCCCCGGAAAAGGTTTGGAGTGGGTGAGCAGCATTTCTCCTTCAGGAGGAGTGACACTT | |
| | TACGCAGACAGTGTTAAGGGTCGGTTCACAATCAGTCGTGATAATTCCAAGAACACTCT | |
| | CTATCTCCAAATGAACTCCCTTCGAGCTGAGGACACTGCTGTTTATTACTGTACTCGGG | |
| | AACTTCTCGGCACTGTGGTTGTTCCTGTGGCTTGGAAGATGCGTGGATACTTCGACTAT | |
| | TGGGGGCAACTCACTCTTGTTACCGTCTCAAGCGCTAGCACCAAAGGACCTAGTGTTTT | |
| | TCCTCTTGCCCCTGAGGTTCAACTCCTTGAATCCGGAGGAGGACTTGTCCAACCAGGCG | |
| | GCAGCCTTAGGCTGTCCTGCGCTGCCTCAGGCTTTACATTCAGTCATTACATGATGGCC | |
| | TGGGTTAGACAGGCACCCGGCAAATGTCTGGAATGGGTCAGCCGAATAGGACCATCAGG | |
| | AGGTCCCACTCACTATGCCGATTCTGTAAAAGGGAGGTTTACAATTTCCAGAGACAATT | |
| | CAAAGAATACCCTTTACCTCCAGATGAACTCATTGAGAGCCGAGGACACAGCCGTATAT | |
| | TATTGCGCAGGTTATGATTCCGGTTACGATTACGTTGCAGTCGCAGGCCCAGCCGAATA | |
| | CTTCCAACATTGGGGTCAGGGAACCCTCGTGACCGTGTCCAGTGCTAGCACCAAAGGAC | |
| | CTAGTGTTTTTCCTCTTGCCCCTTCCTCAAAGTCTACCTCTGGGGGGACAGCCGCTCTG | |
| | GGCTGCCTGGTCAAGGATTATTTCCCAGAGCCTGTCACTGTCAGTTGGAACTCTGGAGC | |
| | CTTGACTTCTGGTGTTCATACATTTCCTGCTGTCCTTCAAAGCAGCGGCTTGTACTCAT | |
| | TGTCTTCTGTTGTGACAGTACCCTCAAGCAGCCTCGGCACTCAGACATACATCTGCAAT | |
| | GTCAACCACAAACCCTCAAATACAAAGGTAGATAAAAAAGTCGAACCAAAGTCTTGTGA | |
| | CAAAACTCACACGTGCCCACCGTGCCCAGCACCTGAACTCCTGGGGGGACCGTCAGTCT | |
| | TCCTCTTCCCCCCAAAACCCAAGGACACCCTCATGATCTCCCGGACCCCTGAGGTCACA | |
| | TGCGTGGTGGTGGACGTGAGCCACGAAGACCCTGAGGTCAAGTTCAACTGGTACGTGGA | |
| | CGGCGTGGAGGTGCATAATGCCAAGACAAAGCCGCGGGAGGAGCAGTACAACAGCACGT | |
| | ACCGTGTGGTCAGCGTCCTCACCGTCCTGCACCAGGACTGGCTGAATGGCAAGGAGTAC | |
| | AAGTGCAAGGTCTCCAACAAAGCCCTCCCAGCCCCCATCGAGAAAACCATCTCCAAAGC | |
| | CAAAGGGCAGCCCCGAGAACCACAGGTGTACACCCTGCCCCCATGCCGGGAGGAGATGA | |
| | CCAAGAACCAGGTCAGCCTGTGGTGCCTGGTCAAAGGCTTCTATCCCAGCGACATCGCC | |
| | GTGGAGTGGGAGAGCAATGGGCAGCCGGAGAACAACTACAAGACCACGCCTCCCGTGCT | |
| | GGACTCCGACGGCTCCTTCTTCCTCTACAGCAAGCTCACCGTGGACAAGAGCAGGTGGC | |
| | AGCAGGGGAACGTCTTCTCATGCTCCGTGATGCATGAGGCTCTGCACAACCACTACACG | |
| | CAGAAGAGCCTCTCCCTGTCTCCGGGTAAA | |
| EPB01 LC | GACATACAGATGACTCAGAGCCCTGGTACACTGAGTGTATCTCCTGGGGAACGCGCCAC | 529 |
| | TCTGTCTTGCCGAGCCTCTCAAAGCGTTAGTTCCAACCTGGCTTGGTATCAGCAAAAAC | |
| | CCGGTCAAGCACCTCGCTTGCTTATCTACGGGGCCTCCACACGGGCAACCGGCATTCCT | |
| | GCACGCTTTAGTGGCTCTGGTAGCGGGACTGAATTCACTTTGACTATATCTTCCATGCA | |
| | GAGTGAGGATTTCGCCGTTTATTATTGTCAACAGTACAATAACTGGCCTCCACTCACTT | |
| | TTGGTGGTGGCACCAAAGTGGAGATAAAGCGTACGGTGGCAGCTCCCAGCGTTTTTATC | |
| | TTTCCCCCAGACATACAGATGACACAAAGTCCAAGTAGCCTTTCTGCATCCGTTGGAGA | |
| | CCGCGTAACCATAACTTGTCGAGCATCACAAAGCATAAGCACTTGGCTTGCTTGGTATC | |
| | AGCAGAAGCCAGGCAAAGCACCCAAGCTGCTGATTTATAAAGCCTCTAATCTCCATACA | |
| | GGAGTTCCCAGCCGTTTCTCAGGCAGCGGGTCTGGAACCGAATTCTCTCTGACCATCTC | |
| | TGGCTTGCAACCTGATGACTTTGCAACATATTACTGTCAACAATATAATTCATATAGTC | |
| | GTACTTTCGGGTGTGGCACTAAGGTGGAAATCAAACGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCCTCTCCCTGTCTCCGGGTAAA | |
| EPB02 HC | GAGGTCCAGCTGGTTGAGTCTGGAGGTGGTGTAGTCAGGCCAGGAGGTTCCCTGCGTCT | 530 |
| | GTCTTGCGCTGCCTCTGGGTTCACTGTGTCTGACTACAGTATGAACTGGGTTAGACAGG | |
| | CTCCAGGGAAAGGTTTGGAATGGATAGGGTTTATTCGCAATAAAGCCAATGCCTATACC | |
| | ACCGAATACAGCGCAAGCGTTAAAGGCAGGTTCACCATCAGTAGGGACGACTCTAAGAA | |
| | TACCCTCTACCTGCAGATGAATAGCTTGAAGACAGAGGATACCGCTGTTTATTACTGCA | |
| | CTACCTACCCCAGGTATCATGCTATGGACAGTTGGGGTCAAGGAACTATGGTCACCGTC | |
| | TCCTCTGCTAGCACCAAAGGACCTAGTGTTTTTCCTCTTGCCCCTGAGGTTCAACTCCT | |
| | TGAATCCGGAGGAGGACTTGTCCAACCAGGCGGCAGCCTTAGGCTGTCCTGCGCTGCCT | |
| | CAGGCTTTACATTCAGTCATTACATGATGGCCTGGGTTAGACAGGCACCCGGCAAATGT | |
| | CTGGAATGGGTCAGCCGAATAGGACCATCAGGAGGTCCCACTCACTATGCCGATTCTGT | |
| | AAAAGGGAGGTTTACAATTTCCAGAGACAATTCAAAGAATACCCTTTACCTCCAGATGA | |
| | ACTCATTGAGAGCCGAGGACACAGCCGTATATTATTGCGCAGGTTATGATTCCGGTTAC | |
| | GATTACGTTGCAGTCGCAGGCCCAGCCGAATACTTCCAACATTGGGGTCAGGGAACCCT | |
| | CGTGACCGTGTCCAGTGCTAGCACCAAAGGACCTAGTGTTTTTCCTCTTGCCCCTTCCT | |
| | CAAAGTCTACCTCTGGGGGGACAGCCGCTCTGGGCTGCCTGGTCAAGGATTATTTCCCA | |
| | GAGCCTGTCACTGTCAGTTGGAACTCTGGAGCCTTGACTTCTGGTGTTCATACATTTCC | |
| | TGCTGTCCTTCAAAGCAGCGGCTTGTACTCATTGTCTTCTGTTGTGACAGTACCCTCAA | |
| | GCAGCCTCGGCACTCAGACATACATCTGCAATGTCAACCACAAACCCTCAAATACAAAG | |
| | GTAGATAAAAAAGTCGAACCAAAGTCTTGTGACAAAACTCACACGTGCCCACCGTGCCC | |
| | AGCACCTGAACTCCTGGGGGGACCGTCAGTCTTCCTCTTCCCCCCAAAACCCAAGGACA | |
| | CCCTCATGATCTCCCGGACCCCTGAGGTCACATGCGTGGTGGTGGACGTGAGCCACGAA | |
| | GACCCTGAGGTCAAGTTCAACTGGTACGTGGACGGCGTGGAGGTGCATAATGCCAAGAC | |
| | AAAGCCGCGGGAGGAGCAGTACAACAGCACGTACCGTGTGGTCAGCGTCCTCACCGTCC | |
| | TGCACCAGGACTGGCTGAATGGCAAGGAGTACAAGTGCAAGGTCTCCAACAAAGCCCTC | |
| | CCAGCCCCCATCGAGAAAACCATCTCCAAAGCCAAAGGGCAGCCCCGAGAACCACAGGT | |
| | GTACACCCTGCCCCCATGCCGGGAGGAGATGACCAAGAACCAGGTCAGCCTGTGGTGCC | |
| | TGGTCAAAGGCTTCTATCCCAGCGACATCGCCGTGGAGTGGGAGAGCAATGGGCAGCCG | |
| | GAGAACAACTACAAGACCACGCCTCCCGTGCTGGACTCCGACGGCTCCTTCTTCCTCTA | |
| | CAGCAAGCTCACCGTGGACAAGAGCAGGTGGCAGCAGGGGAACGTCTTCTCATGCTCCG | |
| | TGATGCATGAGGCTCTGCACAACCACTACACGCAGAAGAGCCTCTCCCTGTCTCCGGGT | |
| | AAA | |
| EPB02 LC | GCCATACAGCTGACTCAATCACCTTCTTCTTTGAGCGCCTCTGTCGGTGATCGCGTTAC | 531 |
| | AATTACATGCCGAGCTTCTCAGTCTATCTCTAATAACCTCCACTGGTATCTCCAGAAAC | |
| | CTGGACAGTCACCACAGCTTCTGATATATTACGGCTTTCAGTCTATAAGTGGAGTCCCA | |
| | AGTAGATTTTCCGGTAGTGGATCTGGCACCGATTTTACTCTTACTATCTCTAGCCTCCA | |
| | ACCTGAAGATTTCGCAACCTATTACTGCCAGCAGGCCAATTCCTGGCCCTTGACCTTCG | |
| | GAGGTGGGACTAAGCTGGAAATCAAACGTACGGTGGCAGCTCCCAGCGTTTTTATCTTT | |
| | CCCCCAGACATACAGATGACACAAAGTCCAAGTAGCCTTTCTGCATCCGTTGGAGACCG | |
| | CGTAACCATAACTTGTCGAGCATCACAAAGCATAAGCACTTGGCTTGCTTGGTATCAGC | |
| | AGAAGCCAGGCAAAGCACCCAAGCTGCTGATTTATAAAGCCTCTAATCTCCATACAGGA | |
| | GTTCCCAGCCGTTTCTCAGGCAGCGGGTCTGGAACCGAATTCTCTCTGACCATCTCTGG | |
| | CTTGCAACCTGATGACTTTGCAACATATTACTGTCAACAATATAATTCATATAGTCGTA | |
| | CTTTCGGGTGTGGCACTAAGGTGGAAATCAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |
| EPB03 HC | CAAGTGCAACTCCTTGAAAGTGGAGGTGGCCTTGTCCAGCCAGGCGGTTCACTGCGTTT | 532 |
| | GAGCTGTGCCGCATCCGGCTTTACCTTTTCCAGTTACACAATGAGTTGGGTCCGTCAAG | |
| | CACCCGGGCAAGCTTTGGAATGGATGGGAACTATTTCATCTGGTGGGACTTACACTTAC | |
| | TATCCCGATAGCGTCAAGGGACGTTTTACCATAAGCCGGGACAACGCTAAAAATTCACT | |
| | CTATCTTCAGATGAACAGTCTTAGGGCTGAGGATACCGCTGTTTATTATTGCGCCCGAG | |
| | AAGCCATTTTCACCTACTGGGGGCGCGGTACACTTGTCACCGTTAGCAGTGCTAGCACC | |
| | AAAGGACCTAGTGTTTTTCCTCTTGCCCCTGAGGTTCAACTCCTTGAATCCGGAGGAGG | |
| | ACTTGTCCAACCAGGCGGCAGCCTTAGGCTGTCCTGCGCTGCCTCAGGCTTTACATTCA | |
| | GTCATTACATGATGGCCTGGGTTAGACAGGCACCCGGCAAATGTCTGGAATGGGTCAGC | |
| | CGAATAGGACCATCAGGAGGTCCCACTCACTATGCCGATTCTGTAAAAGGGAGGTTTAC | |
| | AATTTCCAGAGACAATTCAAAGAATACCCTTTACCTCCAGATGAACTCATTGAGAGCCG | |
| | AGGACACAGCCGTATATTATTGCGCAGGTTATGATTCCGGTTACGATTACGTTGCAGTC | |
| | GCAGGCCCAGCCGAATACTTCCAACATTGGGGTCAGGGAACCCTCGTGACCGTGTCCAG | |
| | TGCTAGCACCAAAGGACCTAGTGTTTTTCCTCTTGCCCCTTCCTCAAAGTCTACCTCTG | |
| | GGGGGACAGCCGCTCTGGGCTGCCTGGTCAAGGATTATTTCCCAGAGCCTGTCACTGTC | |
| | AGTTGGAACTCTGGAGCCTTGACTTCTGGTGTTCATACATTTCCTGCTGTCCTTCAAAG | |
| | CAGCGGCTTGTACTCATTGTCTTCTGTTGTGACAGTACCCTCAAGCAGCCTCGGCACTC | |
| | AGACATACATCTGCAATGTCAACCACAAACCCTCAAATACAAAGGTAGATAAAAAAGTC | |
| | GAACCAAAGTCTTGTGACAAAACTCACACGTGCCCACCGTGCCCAGCACCTGAACTCCT | |
| | GGGGGGACCGTCAGTCTTCCTCTTCCCCCCAAAACCCAAGGACACCCTCATGATCTCCC | |
| | GGACCCCTGAGGTCACATGCGTGGTGGTGGACGTGAGCCACGAAGACCCTGAGGTCAAG | |
| | TTCAACTGGTACGTGGACGGCGTGGAGGTGCATAATGCCAAGACAAAGCCGCGGGAGGA | |
| | GCAGTACAACAGCACGTACCGTGTGGTCAGCGTCCTCACCGTCCTGCACCAGGACTGGC | |
| | TGAATGGCAAGGAGTACAAGTGCAAGGTCTCCAACAAAGCCCTCCCAGCCCCCATCGAG | |
| | AAAACCATCTCCAAAGCCAAAGGGCAGCCCCGAGAACCACAGGTGTACACCCTGCCCCC | |
| | ATGCCGGGAGGAGATGACCAAGAACCAGGTCAGCCTGTGGTGCCTGGTCAAAGGCTTCT | |
| | ATCCCAGCGACATCGCCGTGGAGTGGGAGAGCAATGGGCAGCCGGAGAACAACTACAAG | |
| | ACCACGCCTCCCGTGCTGGACTCCGACGGCTCCTTCTTCCTCTACAGCAAGCTCACCGT | |
| | GGACAAGAGCAGGTGGCAGCAGGGGAACGTCTTCTCATGCTCCGTGATGCATGAGGCTC | |
| | TGCACAACCACTACACGCAGAAGAGCCTCTCCCTGTCTCCGGGTAAA | |
| EPB03 LC | GACATACAACTCACACAAAGTCCAAGCTCTCTTTCCCTTAGTCCAGGAGAACGCGTAAC | 533 |
| | TCTCTCATGTAAAGCCTCCCAGGATATCAATAACTATCTCTCTTGGTACCAACAAAAGC | |
| | CTGACCAAGCTCCTAAGCTGCTTATCAAACGCGCCAATCGGTTGGTAGACGGAGTTCCC | |
| | GACAGGTTCAGTGGGTCAGGCTCAGGTACAGATTTTACCTTGAAGATATCACGCGTGGA | |
| | AGCCGAAGACGTAGGGGTATATTATTGTCTGAAATATGACGAGTTCCCCTATACTTTTG | |
| | GGCAAGGAACTCGCCTGGAAATAAAACGTACGGTGGCAGCTCCCAGCGTTTTTATCTTT | |
| | CCCCCAGACATACAGATGACACAAAGTCCAAGTAGCCTTTCTGCATCCGTTGGAGACCG | |
| | CGTAACCATAACTTGTCGAGCATCACAAAGCATAAGCACTTGGCTTGCTTGGTATCAGC | |
| | AGAAGCCAGGCAAAGCACCCAAGCTGCTGATTTATAAAGCCTCTAATCTCCATACAGGA | |
| | GTTCCCAGCCGTTTCTCAGGCAGCGGGTCTGGAACCGAATTCTCTCTGACCATCTCTGG | |
| | CTTGCAACCTGATGACTTTGCAACATATTACTGTCAACAATATAATTCATATAGTCGTA | |
| | CTTTCGGGTGTGGCACTAAGGTGGAAATCAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |
| EPB04 HC | GAGGTTCAACTCCTTGAATCCGGAGGAGGACTTGTCCAACCAGGCGGCAGCCTTAGGCT | 534 |
| | GTCCTGCGCTGCCTCAGGCTTTACATTCAGTCATTACATGATGGCCTGGGTTAGACAGG | |
| | CACCCGGCAAAGGTCTGGAATGGGTCAGCCGAATAGGACCATCAGGAGGTCCCACTCAC | |
| | TATGCCGATTCTGTAAAAGGGAGGTTTACAATTTCCAGAGACAATTCAAAGAATACCCT | |
| | TTACCTCCAGATGAACTCATTGAGAGCCGAGGACACAGCCGTATATTATTGCGCAGGTT | |
| | ATGATTCCGGTTACGATTACGTTGCAGTCGCAGGCCCAGCCGAATACTTCCAACATTGG | |
| | GGTCAGGGAACCCTCGTGACCGTGTCCAGTGCTAGCACCAAAGGACCTAGTGTTTTTCC | |
| | TCTTGCCCCTGAGGTGCAACTGCTTGAGTCAGGAGGAGGACTGGTTCAACCCGGCGGGA | |
| | GCCTTCGACTCTCATGTGCAGCCTCTGGGTTTACATTTAGTCGTTACCAAATGATGTGG | |
| | GTACGCCAGGCCCCCGGAAAATGTTTGGAGTGGGTGAGCAGCATTTCTCCTTCAGGAGG | |
| | AGTGACACTTTACGCAGACAGTGTTAAGGGTCGGTTCACAATCAGTCGTGATAATTCCA | |
| | AGAACACTCTCTATCTCCAAATGAACTCCCTTCGAGCTGAGGACACTGCTGTTTATTAC | |
| | TGTACTCGGGAACTTCTCGGCACTGTGGTTGTTCCTGTGGCTTGGAAGATGCGTGGATA | |
| | CTTCGACTATTGGGGGCAACTCACTCTTGTTACCGTCTCAAGCGCTAGCACCAAAGGAC | |
| | CTAGTGTTTTTCCTCTTGCCCCTTCCTCAAAGTCTACCTCTGGGGGGACAGCCGCTCTG | |
| | GGCTGCCTGGTCAAGGATTATTTCCCAGAGCCTGTCACTGTCAGTTGGAACTCTGGAGC | |
| | CTTGACTTCTGGTGTTCATACATTTCCTGCTGTCCTTCAAAGCAGCGGCTTGTACTCAT | |
| | TGTCTTCTGTTGTGACAGTACCCTCAAGCAGCCTCGGCACTCAGACATACATCTGCAAT | |
| | GTCAACCACAAACCCTCAAATACAAAGGTAGATAAAAAAGTCGAACCAAAGTCTTGTGA | |
| | CAAAACTCACACGTGCCCACCGTGCCCAGCACCTGAACTCCTGGGGGGACCGTCAGTCT | |
| | TCCTCTTCCCCCCAAAACCCAAGGACACCCTCATGATCTCCCGGACCCCTGAGGTCACA | |
| | TGCGTGGTGGTGGACGTGAGCCACGAAGACCCTGAGGTCAAGTTCAACTGGTACGTGGA | |
| | CGGCGTGGAGGTGCATAATGCCAAGACAAAGCCGCGGGAGGAGCAGTACAACAGCACGT | |
| | ACCGTGTGGTCAGCGTCCTCACCGTCCTGCACCAGGACTGGCTGAATGGCAAGGAGTAC | |
| | AAGTGCAAGGTCTCCAACAAAGCCCTCCCAGCCCCCATCGAGAAAACCATCTCCAAAGC | |
| | CAAAGGGCAGCCCCGAGAACCACAGGTGTACACCCTGCCCCCATGCCGGGAGGAGATGA | |
| | CCAAGAACCAGGTCAGCCTGTGGTGCCTGGTCAAAGGCTTCTATCCCAGCGACATCGCC | |
| | GTGGAGTGGGAGAGCAATGGGCAGCCGGAGAACAACTACAAGACCACGCCTCCCGTGCT | |
| | GGACTCCGACGGCTCCTTCTTCCTCTACAGCAAGCTCACCGTGGACAAGAGCAGGTGGC | |
| | AGCAGGGGAACGTCTTCTCATGCTCCGTGATGCATGAGGCTCTGCACAACCACTACACG | |
| | CAGAAGAGCCTCTCCCTGTCTCCGGGTAAA | |
| EPB04 LC | GACATACAGATGACACAAAGTCCAAGTAGCCTTTCTGCATCCGTTGGAGACCGCGTAAC | 535 |
| | CATAACTTGTCGAGCATCACAAAGCATAAGCACTTGGCTTGCTTGGTATCAGCAGAAGC | |
| | CAGGCAAAGCACCCAAGCTGCTGATTTATAAAGCCTCTAATCTCCATACAGGAGTTCCC | |
| | AGCCGTTTCTCAGGCAGCGGGTCTGGAACCGAATTCTCTCTGACCATCTCTGGCTTGCA | |
| | ACCTGATGACTTTGCAACATATTACTGTCAACAATATAATTCATATAGTCGTACTTTCG | |
| | GGCAAGGCACTAAGGTGGAAATCAAACGTACGGTGGCAGCTCCCAGCGTTTTTATCTTT | |
| | CCCCCAGACATACAGATGACTCAGAGCCCTGGTACACTGAGTGTATCTCCTGGGGAACG | |
| | CGCCACTCTGTCTTGCCGAGCCTCTCAAAGCGTTAGTTCCAACCTGGCTTGGTATCAGC | |
| | AAAAACCCGGTCAAGCACCTCGCTTGCTTATCTACGGGGCCTCCACACGGGCAACCGGC | |
| | ATTCCTGCACGCTTTAGTGGCTCTGGTAGCGGGACTGAATTCACTTTGACTATATCTTC | |
| | CATGCAGAGTGAGGATTTCGCCGTTTATTATTGTCAACAGTACAATAACTGGCCTCCAC | |
| | TCACTTTTGGTTGTGGCACCAAAGTGGAGATAAAGCGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCCTCTCCCTGTCTCCGGGTAAA | |
| EPB05 HC | GAGGTTCAACTCCTTGAATCCGGAGGAGGACTTGTCCAACCAGGCGGCAGCCTTAGGCT | 536 |
| | GTCCTGCGCTGCCTCAGGCTTTACATTCAGTCATTACATGATGGCCTGGGTTAGACAGG | |
| | CACCCGGCAAAGGTCTGGAATGGGTCAGCCGAATAGGACCATCAGGAGGTCCCACTCAC | |
| | TATGCCGATTCTGTAAAAGGGAGGTTTACAATTTCCAGAGACAATTCAAAGAATACCCT | |
| | TTACCTCCAGATGAACTCATTGAGAGCCGAGGACACAGCCGTATATTATTGCGCAGGTT | |
| | ATGATTCCGGTTACGATTACGTTGCAGTCGCAGGCCCAGCCGAATACTTCCAACATTGG | |
| | GGTCAGGGAACCCTCGTGACCGTGTCCAGTGCTAGCACCAAAGGACCTAGTGTTTTTCC | |
| | TCTTGCCCCTGAGGTCCAGCTGGTTGAGTCTGGAGGTGGTGTAGTCAGGCCAGGAGGTT | |
| | CCCTGCGTCTGTCTTGCGCTGCCTCTGGGTTCACTGTGTCTGACTACAGTATGAACTGG | |
| | GTTAGACAGGCTCCAGGGAAATGTTTGGAATGGATAGGGTTTATTCGCAATAAAGCCAA | |
| | TGCCTATACCACCGAATACAGCGCAAGCGTTAAAGGCAGGTTCACCATCAGTAGGGACG | |
| | ACTCTAAGAATACCCTCTACCTGCAGATGAATAGCTTGAAGACAGAGGATACCGCTGTT | |
| | TATTACTGCACTACCTACCCCAGGTATCATGCTATGGACAGTTGGGGTCAAGGAACTAT | |
| | GGTCACCGTCTCCTCTGCTAGCACCAAAGGACCTAGTGTTTTTCCTCTTGCCCCTTCCT | |
| | CAAAGTCTACCTCTGGGGGGACAGCCGCTCTGGGCTGCCTGGTCAAGGATTATTTCCCA | |
| | GAGCCTGTCACTGTCAGTTGGAACTCTGGAGCCTTGACTTCTGGTGTTCATACATTTCC | |
| | TGCTGTCCTTCAAAGCAGCGGCTTGTACTCATTGTCTTCTGTTGTGACAGTACCCTCAA | |
| | GCAGCCTCGGCACTCAGACATACATCTGCAATGTCAACCACAAACCCTCAAATACAAAG | |
| | GTAGATAAAAAAGTCGAACCAAAGTCTTGTGACAAAACTCACACGTGCCCACCGTGCCC | |
| | AGCACCTGAACTCCTGGGGGGACCGTCAGTCTTCCTCTTCCCCCCAAAACCCAAGGACA | |
| | CCCTCATGATCTCCCGGACCCCTGAGGTCACATGCGTGGTGGTGGACGTGAGCCACGAA | |
| | GACCCTGAGGTCAAGTTCAACTGGTACGTGGACGGCGTGGAGGTGCATAATGCCAAGAC | |
| | AAAGCCGCGGGAGGAGCAGTACAACAGCACGTACCGTGTGGTCAGCGTCCTCACCGTCC | |
| | TGCACCAGGACTGGCTGAATGGCAAGGAGTACAAGTGCAAGGTCTCCAACAAAGCCCTC | |
| | CCAGCCCCCATCGAGAAAACCATCTCCAAAGCCAAAGGGCAGCCCCGAGAACCACAGGT | |
| | GTACACCCTGCCCCCATGCCGGGAGGAGATGACCAAGAACCAGGTCAGCCTGTGGTGCC | |
| | TGGTCAAAGGCTTCTATCCCAGCGACATCGCCGTGGAGTGGGAGAGCAATGGGCAGCCG | |
| | GAGAACAACTACAAGACCACGCCTCCCGTGCTGGACTCCGACGGCTCCTTCTTCCTCTA | |
| | CAGCAAGCTCACCGTGGACAAGAGCAGGTGGCAGCAGGGGAACGTCTTCTCATGCTCCG | |
| | TGATGCATGAGGCTCTGCACAACCACTACACGCAGAAGAGCCTCTCCCTGTCTCCGGGT | |
| | AAA | |
| EPB05 LC | GACATACAGATGACACAAAGTCCAAGTAGCCTTTCTGCATCCGTTGGAGACCGCGTAAC | 537 |
| | CATAACTTGTCGAGCATCACAAAGCATAAGCACTTGGCTTGCTTGGTATCAGCAGAAGC | |
| | CAGGCAAAGCACCCAAGCTGCTGATTTATAAAGCCTCTAATCTCCATACAGGAGTTCCC | |
| | AGCCGTTTCTCAGGCAGCGGGTCTGGAACCGAATTCTCTCTGACCATCTCTGGCTTGCA | |
| | ACCTGATGACTTTGCAACATATTACTGTCAACAATATAATTCATATAGTCGTACTTTCG | |
| | GGCAAGGCACTAAGGTGGAAATCAAACGTACGGTGGCAGCTCCCAGCGTTTTTATCTTT | |
| | CCCCCAGCCATACAGCTGACTCAATCACCTTCTTCTTTGAGCGCCTCTGTCGGTGATCG | |
| | CGTTACAATTACATGCCGAGCTTCTCAGTCTATCTCTAATAACCTCCACTGGTATCTCC | |
| | AGAAACCTGGACAGTCACCACAGCTTCTGATATATTACGGCTTTCAGTCTATAAGTGGA | |
| | GTCCCAAGTAGATTTTCCGGTAGTGGATCTGGCACCGATTTTACTCTTACTATCTCTAG | |
| | CCTCCAACCTGAAGATTTCGCAACCTATTACTGCCAGCAGGCCAATTCCTGGCCCTTGA | |
| | CCTTCGGATGTGGGACTAAGCTGGAAATCAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |
| EPB06 HC | GAGGTTCAACTCCTTGAATCCGGAGGAGGACTTGTCCAACCAGGCGGCAGCCTTAGGCT | 538 |
| | GTCCTGCGCTGCCTCAGGCTTTACATTCAGTCATTACATGATGGCCTGGGTTAGACAGG | |
| | CACCCGGCAAAGGTCTGGAATGGGTCAGCCGAATAGGACCATCAGGAGGTCCCACTCAC | |
| | TATGCCGATTCTGTAAAAGGGAGGTTTACAATTTCCAGAGACAATTCAAAGAATACCCT | |
| | TTACCTCCAGATGAACTCATTGAGAGCCGAGGACACAGCCGTATATTATTGCGCAGGTT | |
| | ATGATTCCGGTTACGATTACGTTGCAGTCGCAGGCCCAGCCGAATACTTCCAACATTGG | |
| | GGTCAGGGAACCCTCGTGACCGTGTCCAGTGCTAGCACCAAAGGACCTAGTGTTTTTCC | |
| | TCTTGCCCCTCAAGTGCAACTCCTTGAAAGTGGAGGTGGCCTTGTCCAGCCAGGCGGTT | |
| | CACTGCGTTTGAGCTGTGCCGCATCCGGCTTTACCTTTTCCAGTTACACAATGAGTTGG | |
| | GTCCGTCAAGCACCCGGGCAATGTTTGGAATGGATGGGAACTATTTCATCTGGTGGGAC | |
| | TTACACTTACTATCCCGATAGCGTCAAGGGACGTTTTACCATAAGCCGGGACAACGCTA | |
| | AAAATTCACTCTATCTTCAGATGAACAGTCTTAGGGCTGAGGATACCGCTGTTTATTAT | |
| | TGCGCCCGAGAAGCCATTTTCACCTACTGGGGGCGCGGTACACTTGTCACCGTTAGCAG | |
| | TGCTAGCACCAAAGGACCTAGTGTTTTTCCTCTTGCCCCTTCCTCAAAGTCTACCTCTG | |
| | GGGGGACAGCCGCTCTGGGCTGCCTGGTCAAGGATTATTTCCCAGAGCCTGTCACTGTC | |
| | AGTTGGAACTCTGGAGCCTTGACTTCTGGTGTTCATACATTTCCTGCTGTCCTTCAAAG | |
| | CAGCGGCTTGTACTCATTGTCTTCTGTTGTGACAGTACCCTCAAGCAGCCTCGGCACTC | |
| | AGACATACATCTGCAATGTCAACCACAAACCCTCAAATACAAAGGTAGATAAAAAAGTC | |
| | GAACCAAAGTCTTGTGACAAAACTCACACGTGCCCACCGTGCCCAGCACCTGAACTCCT | |
| | GGGGGGACCGTCAGTCTTCCTCTTCCCCCCAAAACCCAAGGACACCCTCATGATCTCCC | |
| | GGACCCCTGAGGTCACATGCGTGGTGGTGGACGTGAGCCACGAAGACCCTGAGGTCAAG | |
| | TTCAACTGGTACGTGGACGGCGTGGAGGTGCATAATGCCAAGACAAAGCCGCGGGAGGA | |
| | GCAGTACAACAGCACGTACCGTGTGGTCAGCGTCCTCACCGTCCTGCACCAGGACTGGC | |
| | TGAATGGCAAGGAGTACAAGTGCAAGGTCTCCAACAAAGCCCTCCCAGCCCCCATCGAG | |
| | AAAACCATCTCCAAAGCCAAAGGGCAGCCCCGAGAACCACAGGTGTACACCCTGCCCCC | |
| | ATGCCGGGAGGAGATGACCAAGAACCAGGTCAGCCTGTGGTGCCTGGTCAAAGGCTTCT | |
| | ATCCCAGCGACATCGCCGTGGAGTGGGAGAGCAATGGGCAGCCGGAGAACAACTACAAG | |
| | ACCACGCCTCCCGTGCTGGACTCCGACGGCTCCTTCTTCCTCTACAGCAAGCTCACCGT | |
| | GGACAAGAGCAGGTGGCAGCAGGGGAACGTCTTCTCATGCTCCGTGATGCATGAGGCTC | |
| | TGCACAACCACTACACGCAGAAGAGCCTCTCCCTGTCTCCGGGTAAA | |
| EPB06 LC | GACATACAGATGACACAAAGTCCAAGTAGCCTTTCTGCATCCGTTGGAGACCGCGTAAC | 539 |
| | CATAACTTGTCGAGCATCACAAAGCATAAGCACTTGGCTTGCTTGGTATCAGCAGAAGC | |
| | CAGGCAAAGCACCCAAGCTGCTGATTTATAAAGCCTCTAATCTCCATACAGGAGTTCCC | |
| | AGCCGTTTCTCAGGCAGCGGGTCTGGAACCGAATTCTCTCTGACCATCTCTGGCTTGCA | |
| | ACCTGATGACTTTGCAACATATTACTGTCAACAATATAATTCATATAGTCGTACTTTCG | |
| | GGCAAGGCACTAAGGTGGAAATCAAACGTACGGTGGCAGCTCCCAGCGTTTTTATCTTT | |
| | CCCCCAGACATACAACTCACACAAAGTCCAAGCTCTCTTTCCCTTAGTCCAGGAGAACG | |
| | CGTAACTCTCTCATGTAAAGCCTCCCAGGATATCAATAACTATCTCTCTTGGTACCAAC | |
| | AAAAGCCTGACCAAGCTCCTAAGCTGCTTATCAAACGCGCCAATCGGTTGGTAGACGGA | |
| | GTTCCCGACAGGTTCAGTGGGTCAGGCTCAGGTACAGATTTTACCTTGAAGATATCACG | |
| | CGTGGAAGCCGAAGACGTAGGGGTATATTATTGTCTGAAATATGACGAGTTCCCCTATA | |
| | CTTTTGGGTGTGGAACTCGCCTGGAAATAAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |
| EPB07 HC | GAGGTCCAGCTGGTTGAGTCTGGAGGTGGTGTAGTCAGGCCAGGAGGTTCCCTGCGTCT | 540 |
| | GTCTTGCGCTGCCTCTGGGTTCACTGTGTCTGACTACAGTATGAACTGGGTTAGACAGG | |
| | CTCCAGGGAAAGGTTTGGAATGGATAGGGTTTATTCGCAATAAAGCCAATGCCTATACC | |
| | ACCGAATACAGCGCAAGCGTTAAAGGCAGGTTCACCATCAGTAGGGACGACTCTAAGAA | |
| | TACCCTCTACCTGCAGATGAATAGCTTGAAGACAGAGGATACCGCTGTTTATTACTGCA | |
| | CTACCTACCCCAGGTATCATGCTATGGACAGTTGGGGTCAAGGAACTATGGTCACCGTC | |
| | TCCTCTGCTAGCACCAAAGGACCTAGTGTTTTTCCTCTTGCCCCTGAGGTGCAACTGCT | |
| | TGAGTCAGGAGGAGGACTGGTTCAACCCGGCGGGAGCCTTCGACTCTCATGTGCAGCCT | |
| | CTGGGTTTACATTTAGTCGTTACCAAATGATGTGGGTACGCCAGGCCCCCGGAAAATGT | |
| | TTGGAGTGGGTGAGCAGCATTTCTCCTTCAGGAGGAGTGACACTTTACGCAGACAGTGT | |
| | TAAGGGTCGGTTCACAATCAGTCGTGATAATTCCAAGAACACTCTCTATCTCCAAATGA | |
| | ACTCCCTTCGAGCTGAGGACACTGCTGTTTATTACTGTACTCGGGAACTTCTCGGCACT | |
| | GTGGTTGTTCCTGTGGCTTGGAAGATGCGTGGATACTTCGACTATTGGGGGCAACTCAC | |
| | TCTTGTTACCGTCTCAAGCGCTAGCACCAAAGGACCTAGTGTTTTTCCTCTTGCCCCTT | |
| | CCTCAAAGTCTACCTCTGGGGGGACAGCCGCTCTGGGCTGCCTGGTCAAGGATTATTTC | |
| | CCAGAGCCTGTCACTGTCAGTTGGAACTCTGGAGCCTTGACTTCTGGTGTTCATACATT | |
| | TCCTGCTGTCCTTCAAAGCAGCGGCTTGTACTCATTGTCTTCTGTTGTGACAGTACCCT | |
| | CAAGCAGCCTCGGCACTCAGACATACATCTGCAATGTCAACCACAAACCCTCAAATACA | |
| | AAGGTAGATAAAAAAGTCGAACCAAAGTCTTGTGACAAAACTCACACGTGCCCACCGTG | |
| | CCCAGCACCTGAACTCCTGGGGGGACCGTCAGTCTTCCTCTTCCCCCCAAAACCCAAGG | |
| | ACACCCTCATGATCTCCCGGACCCCTGAGGTCACATGCGTGGTGGTGGACGTGAGCCAC | |
| | GAAGACCCTGAGGTCAAGTTCAACTGGTACGTGGACGGCGTGGAGGTGCATAATGCCAA | |
| | GACAAAGCCGCGGGAGGAGCAGTACAACAGCACGTACCGTGTGGTCAGCGTCCTCACCG | |
| | TCCTGCACCAGGACTGGCTGAATGGCAAGGAGTACAAGTGCAAGGTCTCCAACAAAGCC | |
| | CTCCCAGCCCCCATCGAGAAAACCATCTCCAAAGCCAAAGGGCAGCCCCGAGAACCACA | |
| | GGTGTACACCCTGCCCCCATGCCGGGAGGAGATGACCAAGAACCAGGTCAGCCTGTGGT | |
| | GCCTGGTCAAAGGCTTCTATCCCAGCGACATCGCCGTGGAGTGGGAGAGCAATGGGCAG | |
| | CCGGAGAACAACTACAAGACCACGCCTCCCGTGCTGGACTCCGACGGCTCCTTCTTCCT | |
| | CTACAGCAAGCTCACCGTGGACAAGAGCAGGTGGCAGCAGGGGAACGTCTTCTCATGCT | |
| | CCGTGATGCATGAGGCTCTGCACAACCACTACACGCAGAAGAGCCTCTCCCTGTCTCCG | |
| | GGTAAA | |
| EPB07 LC | GCCATACAGCTGACTCAATCACCTTCTTCTTTGAGCGCCTCTGTCGGTGATCGCGTTAC | 541 |
| | AATTACATGCCGAGCTTCTCAGTCTATCTCTAATAACCTCCACTGGTATCTCCAGAAAC | |
| | CTGGACAGTCACCACAGCTTCTGATATATTACGGCTTTCAGTCTATAAGTGGAGTCCCA | |
| | AGTAGATTTTCCGGTAGTGGATCTGGCACCGATTTTACTCTTACTATCTCTAGCCTCCA | |
| | ACCTGAAGATTTCGCAACCTATTACTGCCAGCAGGCCAATTCCTGGCCCTTGACCTTCG | |
| | GAGGTGGGACTAAGCTGGAAATCAAACGTACGGTGGCAGCTCCCAGCGTTTTTATCTTT | |
| | CCCCCAGACATACAGATGACTCAGAGCCCTGGTACACTGAGTGTATCTCCTGGGGAACG | |
| | CGCCACTCTGTCTTGCCGAGCCTCTCAAAGCGTTAGTTCCAACCTGGCTTGGTATCAGC | |
| | AAAAACCCGGTCAAGCACCTCGCTTGCTTATCTACGGGGCCTCCACACGGGCAACCGGC | |
| | ATTCCTGCACGCTTTAGTGGCTCTGGTAGCGGGACTGAATTCACTTTGACTATATCTTC | |
| | CATGCAGAGTGAGGATTTCGCCGTTTATTATTGTCAACAGTACAATAACTGGCCTCCAC | |
| | TCACTTTTGGTTGTGGCACCAAAGTGGAGATAAAGCGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCCTCTCCCTGTCTCCGGGTAAA | |
| EPB08 HC | CAAGTGCAACTCCTTGAAAGTGGAGGTGGCCTTGTCCAGCCAGGCGGTTCACTGCGTTT | 542 |
| | GAGCTGTGCCGCATCCGGCTTTACCTTTTCCAGTTACACAATGAGTTGGGTCCGTCAAG | |
| | CACCCGGGCAAGCTTTGGAATGGATGGGAACTATTTCATCTGGTGGGACTTACACTTAC | |
| | TATCCCGATAGCGTCAAGGGACGTTTTACCATAAGCCGGGACAACGCTAAAAATTCACT | |
| | CTATCTTCAGATGAACAGTCTTAGGGCTGAGGATACCGCTGTTTATTATTGCGCCCGAG | |
| | AAGCCATTTTCACCTACTGGGGGCGCGGTACACTTGTCACCGTTAGCAGTGCTAGCACC | |
| | AAAGGACCTAGTGTTTTTCCTCTTGCCCCTGAGGTGCAACTGCTTGAGTCAGGAGGAGG | |
| | ACTGGTTCAACCCGGCGGGAGCCTTCGACTCTCATGTGCAGCCTCTGGGTTTACATTTA | |
| | GTCGTTACCAAATGATGTGGGTACGCCAGGCCCCCGGAAAATGTTTGGAGTGGGTGAGC | |
| | AGCATTTCTCCTTCAGGAGGAGTGACACTTTACGCAGACAGTGTTAAGGGTCGGTTCAC | |
| | AATCAGTCGTGATAATTCCAAGAACACTCTCTATCTCCAAATGAACTCCCTTCGAGCTG | |
| | AGGACACTGCTGTTTATTACTGTACTCGGGAACTTCTCGGCACTGTGGTTGTTCCTGTG | |
| | GCTTGGAAGATGCGTGGATACTTCGACTATTGGGGGCAACTCACTCTTGTTACCGTCTC | |
| | AAGCGCTAGCACCAAAGGACCTAGTGTTTTTCCTCTTGCCCCTTCCTCAAAGTCTACCT | |
| | CTGGGGGGACAGCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCTGCTGTCCTTCA | |
| | AAGCAGCGGCTTGTACTCATTGTCTTCTGTTGTGACAGTACCCTCAAGCAGCCTCGGCA | |
| | CTCAGACATACATCTGCAATGTCAACCACAAACCCTCAAATACAAAGGTAGATAAAAAA | |
| | GTCGAACCAAAGTCTTGTGACAAAACTCACACGTGCCCACCGTGCCCAGCACCTGAACT | |
| | CCTGGGGGGACCGTCAGTCTTCCTCTTCCCCCCAAAACCCAAGGACACCCTCATGATCT | |
| | CCCGGACCCCTGAGGTCACATGCGTGGTGGTGGACGTGAGCCACGAAGACCCTGAGGTC | |
| | AAGTTCAACTGGTACGTGGACGGCGTGGAGGTGCATAATGCCAAGACAAAGCCGCGGGA | |
| | GGAGCAGTACAACAGCACGTACCGTGTGGTCAGCGTCCTCACCGTCCTGCACCAGGACT | |
| | GGCTGAATGGCAAGGAGTACAAGTGCAAGGTCTCCAACAAAGCCCTCCCAGCCCCCATC | |
| | GAGAAAACCATCTCCAAAGCCAAAGGGCAGCCCCGAGAACCACAGGTGTACACCCTGCC | |
| | CCCATGCCGGGAGGAGATGACCAAGAACCAGGTCAGCCTGTGGTGCCTGGTCAAAGGCT | |
| | TCTATCCCAGCGACATCGCCGTGGAGTGGGAGAGCAATGGGCAGCCGGAGAACAACTAC | |
| | AAGACCACGCCTCCCGTGCTGGACTCCGACGGCTCCTTCTTCCTCTACAGCAAGCTCAC | |
| | CGTGGACAAGAGCAGGTGGCAGCAGGGGAACGTCTTCTCATGCTCCGTGATGCATGAGG | |
| | CTCTGCACAACCACTACACGCAGAAGAGCCTCTCCCTGTCTCCGGGTAAA | |
| EPB08 LC | GACATACAACTCACACAAAGTCCAAGCTCTCTTTCCCTTAGTCCAGGAGAACGCGTAAC | 543 |
| | TCTCTCATGTAAAGCCTCCCAGGATATCAATAACTATCTCTCTTGGTACCAACAAAAGC | |
| | CTGACCAAGCTCCTAAGCTGCTTATCAAACGCGCCAATCGGTTGGTAGACGGAGTTCCC | |
| | GACAGGTTCAGTGGGTCAGGCTCAGGTACAGATTTTACCTTGAAGATATCACGCGTGGA | |
| | AGCCGAAGACGTAGGGGTATATTATTGTCTGAAATATGACGAGTTCCCCTATACTTTTG | |
| | GGCAAGGAACTCGCCTGGAAATAAAACGTACGGTGGCAGCTCCCAGCGTTTTTATCTTT | |
| | CCCCCAGACATACAGATGACTCAGAGCCCTGGTACACTGAGTGTATCTCCTGGGGAACG | |
| | CGCCACTCTGTCTTGCCGAGCCTCTCAAAGCGTTAGTTCCAACCTGGCTTGGTATCAGC | |
| | AAAAACCCGGTCAAGCACCTCGCTTGCTTATCTACGGGGCCTCCACACGGGCAACCGGC | |
| | ATTCCTGCACGCTTTAGTGGCTCTGGTAGCGGGACTGAATTCACTTTGACTATATCTTC | |
| | CATGCAGAGTGAGGATTTCGCCGTTTATTATTGTCAACAGTACAATAACTGGCCTCCAC | |
| | TCACTTTTGGTTGTGGCACCAAAGTGGAGATAAAGCGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCCTCTCCCTGTCTCCGGGTAAA | |
| EPB09 HC | GAGGTGCAACTGCTTGAGTCAGGAGGAGGACTGGTTCAACCCGGCGGGAGCCTTCGACT | 544 |
| | CTCATGTGCAGCCTCTGGGTTTACATTTAGTCGTTACCAAATGATGTGGGTACGCCAGG | |
| | CCCCCGGAAAAGGTTTGGAGTGGGTGAGCAGCATTTCTCCTTCAGGAGGAGTGACACTT | |
| | TACGCAGACAGTGTTAAGGGTCGGTTCACAATCAGTCGTGATAATTCCAAGAACACTCT | |
| | CTATCTCCAAATGAACTCCCTTCGAGCTGAGGACACTGCTGTTTATTACTGTACTCGGG | |
| | AACTTCTCGGCACTGTGGTTGTTCCTGTGGCTTGGAAGATGCGTGGATACTTCGACTAT | |
| | TGGGGGCAACTCACTCTTGTTACCGTCTCAAGCGCTAGCACCAAAGGACCTAGTGTTTT | |
| | TCCTCTTGCCCCTGAGGTCCAGCTGGTTGAGTCTGGAGGTGGTGTAGTCAGGCCAGGAG | |
| | GTTCCCTGCGTCTGTCTTGCGCTGCCTCTGGGTTCACTGTGTCTGACTACAGTATGAAC | |
| | TGGGTTAGACAGGCTCCAGGGAAATGTTTGGAATGGATAGGGTTTATTCGCAATAAAGC | |
| | CAATGCCTATACCACCGAATACAGCGCAAGCGTTAAAGGCAGGTTCACCATCAGTAGGG | |
| | ACGACTCTAAGAATACCCTCTACCTGCAGATGAATAGCTTGAAGACAGAGGATACCGCT | |
| | GTTTATTACTGCACTACCTACCCCAGGTATCATGCTATGGACAGTTGGGGTCAAGGAAC | |
| | TATGGTCACCGTCTCCTCTGCTAGCACCAAAGGACCTAGTGTTTTTCCTCTTGCCCCTT | |
| | CCTCAAAGTCTACCTCTGGGGGGACAGCCGCTCTGGGCTGCCTGGTCAAGGATTATTTC | |
| | CCAGAGCCTGTCACTGTCAGTTGGAACTCTGGAGCCTTGACTTCTGGTGTTCATACATT | |
| | TCCTGCTGTCCTTCAAAGCAGCGGCTTGTACTCATTGTCTTCTGTTGTGACAGTACCCT | |
| | CAAGCAGCCTCGGCACTCAGACATACATCTGCAATGTCAACCACAAACCCTCAAATACA | |
| | AAGGTAGATAAAAAAGTCGAACCAAAGTCTTGTGACAAAACTCACACGTGCCCACCGTG | |
| | CCCAGCACCTGAACTCCTGGGGGGACCGTCAGTCTTCCTCTTCCCCCCAAAACCCAAGG | |
| | ACACCCTCATGATCTCCCGGACCCCTGAGGTCACATGCGTGGTGGTGGACGTGAGCCAC | |
| | GAAGACCCTGAGGTCAAGTTCAACTGGTACGTGGACGGCGTGGAGGTGCATAATGCCAA | |
| | GACAAAGCCGCGGGAGGAGCAGTACAACAGCACGTACCGTGTGGTCAGCGTCCTCACCG | |
| | TCCTGCACCAGGACTGGCTGAATGGCAAGGAGTACAAGTGCAAGGTCTCCAACAAAGCC | |
| | CTCCCAGCCCCCATCGAGAAAACCATCTCCAAAGCCAAAGGGCAGCCCCGAGAACCACA | |
| | GGTGTACACCCTGCCCCCATGCCGGGAGGAGATGACCAAGAACCAGGTCAGCCTGTGGT | |
| | GCCTGGTCAAAGGCTTCTATCCCAGCGACATCGCCGTGGAGTGGGAGAGCAATGGGCAG | |
| | CCGGAGAACAACTACAAGACCACGCCTCCCGTGCTGGACTCCGACGGCTCCTTCTTCCT | |
| | CTACAGCAAGCTCACCGTGGACAAGAGCAGGTGGCAGCAGGGGAACGTCTTCTCATGCT | |
| | CCGTGATGCATGAGGCTCTGCACAACCACTACACGCAGAAGAGCCTCTCCCTGTCTCCG | |
| | GGTAAA | |
| EPB09 LC | GACATACAGATGACTCAGAGCCCTGGTACACTGAGTGTATCTCCTGGGGAACGCGCCAC | 545 |
| | TCTGTCTTGCCGAGCCTCTCAAAGCGTTAGTTCCAACCTGGCTTGGTATCAGCAAAAAC | |
| | CCGGTCAAGCACCTCGCTTGCTTATCTACGGGGCCTCCACACGGGCAACCGGCATTCCT | |
| | GCACGCTTTAGTGGCTCTGGTAGCGGGACTGAATTCACTTTGACTATATCTTCCATGCA | |
| | GAGTGAGGATTTCGCCGTTTATTATTGTCAACAGTACAATAACTGGCCTCCACTCACTT | |
| | TTGGTGGTGGCACCAAAGTGGAGATAAAGCGTACGGTGGCAGCTCCCAGCGTTTTTATC | |
| | TTTCCCCCAGCCATACAGCTGACTCAATCACCTTCTTCTTTGAGCGCCTCTGTCGGTGA | |
| | TCGCGTTACAATTACATGCCGAGCTTCTCAGTCTATCTCTAATAACCTCCACTGGTATC | |
| | TCCAGAAACCTGGACAGTCACCACAGCTTCTGATATATTACGGCTTTCAGTCTATAAGT | |
| | GGAGTCCCAAGTAGATTTTCCGGTAGTGGATCTGGCACCGATTTTACTCTTACTATCTC | |
| | TAGCCTCCAACCTGAAGATTTCGCAACCTATTACTGCCAGCAGGCCAATTCCTGGCCCT | |
| | TGACCTTCGGATGTGGGACTAAGCTGGAAATCAAACGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCDCTCTCCCTGTCTCCGGGTAAA | |
| EPB10 HC | CAAGTGCAACTCCTTGAAAGTGGAGGTGGCCTTGTCCAGCCAGGCGGTTCACTGCGTTT | 546 |
| | GAGCTGTGCCGCATCCGGCTTTACCTTTTCCAGTTACACAATGAGTTGGGTCCGTCAAG | |
| | CACCCGGGCAAGCTTTGGAATGGATGGGAACTATTTCATCTGGTGGGACTTACACTTAC | |
| | TATCCCGATAGCGTCAAGGGACGTTTTACCATAAGCCGGGACAACGCTAAAAATTCACT | |
| | CTATCTTCAGATGAACAGTCTTAGGGCTGAGGATACCGCTGTTTATTATTGCGCCCGAG | |
| | AAGCCATTTTCACCTACTGGGGGCGCGGTACACTTGTCACCGTTAGCAGTGCTAGCACC | |
| | AAAGGACCTAGTGTTTTTCCTCTTGCCCCTGAGGTCCAGCTGGTTGAGTCTGGAGGTGG | |
| | TGTAGTCAGGCCAGGAGGTTCCCTGCGTCTGTCTTGCGCTGCCTCTGGGTTCACTGTGT | |
| | CTGACTACAGTATGAACTGGGTTAGACAGGCTCCAGGGAAATGTTTGGAATGGATAGGG | |
| | TTTATTCGCAATAAAGCCAATGCCTATACCACCGAATACAGCGCAAGCGTTAAAGGCAG | |
| | GTTCACCATCAGTAGGGACGACTCTAAGAATACCCTCTACCTGCAGATGAATAGCTTGA | |
| | AGACAGAGGATACCGCTGTTTATTACTGCACTACCTACCCCAGGTATCATGCTATGGAC | |
| | AGTTGGGGTCAAGGAACTATGGTCACCGTCTCCTCTGCTAGCACCAAAGGACCTAGTGT | |
| | TTTTCCTCTTGCCCCTTCCTCAAAGTCTACCTCTGGGGGGACAGCCGCTCTGGGCTGCC | |
| | TGGTCAAGGATTATTTCCCAGAGCCTGTCACTGTCAGTTGGAACTCTGGAGCCTTGACT | |
| | TCTGGTGTTCATACATTTCCTGCTGTCCTTCAAAGCAGCGGCTTGTACTCATTGTCTTC | |
| | TGTTGTGACAGTACCCTCAAGCAGCCTCGGCACTCAGACATACATCTGCAATGTCAACC | |
| | ACAAACCCTCAAATACAAAGGTAGATAAAAAAGTCGAACCAAAGTCTTGTGACAAAACT | |
| | CACACGTGCCCACCGTGCCCAGCACCTGAACTCCTGGGGGGACCGTCAGTCTTCCTCTT | |
| | CCCCCCAAAACCCAAGGACACCCTCATGATCTCCCGGACCCCTGAGGTCACATGCGTGG | |
| | TGGTGGACGTGAGCCACGAAGACCCTGAGGTCAAGTTCAACTGGTACGTGGACGGCGTG | |
| | GAGGTGCATAATGCCAAGACAAAGCCGCGGGAGGAGCAGTACAACAGCACGTACCGTGT | |
| | GGTCAGCGTCCTCACCGTCCTGCACCAGGACTGGCTGAATGGCAAGGAGTACAAGTGCA | |
| | AGGTCTCCAACAAAGCCCTCCCAGCCCCCATCGAGAAAACCATCTCCAAAGCCAAAGGG | |
| | CAGCCCCGAGAACCACAGGTGTACACCCTGCCCCCATGCCGGGAGGAGATGACCAAGAA | |
| | CCAGGTCAGCCTGTGGTGCCTGGTCAAAGGCTTCTATCCCAGCGACATCGCCGTGGAGT | |
| | GGGAGAGCAATGGGCAGCCGGAGAACAACTACAAGACCACGCCTCCCGTGCTGGACTCC | |
| | GACGGCTCCTTCTTCCTCTACAGCAAGCTCACCGTGGACAAGAGCAGGTGGCAGCAGGG | |
| | GAACGTCTTCTCATGCTCCGTGATGCATGAGGCTCTGCACAACCACTACACGCAGAAGA | |
| | GCCTCTCCCTGTCTCCGGGTAAA | |
| EPB10 LC | GACATACAACTCACACAAAGTCCAAGCTCTCTTTCCCTTAGTCCAGGAGAACGCGTAAC | 547 |
| | TCTCTCATGTAAAGCCTCCCAGGATATCAATAACTATCTCTCTTGGTACCAACAAAAGC | |
| | CTGACCAAGCTCCTAAGCTGCTTATCAAACGCGCCAATCGGTTGGTAGACGGAGTTCCC | |
| | GACAGGTTCAGTGGGTCAGGCTCAGGTACAGATTTTACCTTGAAGATATCACGCGTGGA | |
| | AGCCGAAGACGTAGGGGTATATTATTGTCTGAAATATGACGAGTTCCCCTATACTTTTG | |
| | GGCAAGGAACTCGCCTGGAAATAAAACGTACGGTGGCAGCTCCCAGCGTTTTTATCTTT | |
| | CCCCCAGCCATACAGCTGACTCAATCACCTTCTTCTTTGAGCGCCTCTGTCGGTGATCG | |
| | CGTTACAATTACATGCCGAGCTTCTCAGTCTATCTCTAATAACCTCCACTGGTATCTCC | |
| | AGAAACCTGGACAGTCACCACAGCTTCTGATATATTACGGCTTTCAGTCTATAAGTGGA | |
| | GTCCCAAGTAGATTTTCCGGTAGTGGATCTGGCACCGATTTTACTCTTACTATCTCTAG | |
| | CCTCCAACCTGAAGATTTCGCAACCTATTACTGCCAGCAGGCCAATTCCTGGCCCTTGA | |
| | CCTTCGGATGTGGGACTAAGCTGGAAATCAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |
| EPB11 HC | GAGGTGCAACTGCTTGAGTCAGGAGGAGGACTGGTTCAACCCGGCGGGAGCCTTCGACT | 548 |
| | CTCATGTGCAGCCTCTGGGTTTACATTTAGTCGTTACCAAATGATGTGGGTACGCCAGG | |
| | CCCCCGGAAAAGGTTTGGAGTGGGTGAGCAGCATTTCTCCTTCAGGAGGAGTGACACTT | |
| | TACGCAGACAGTGTTAAGGGTCGGTTCACAATCAGTCGTGATAATTCCAAGAACACTCT | |
| | CTATCTCCAAATGAACTCCCTTCGAGCTGAGGACACTGCTGTTTATTACTGTACTCGGG | |
| | AACTTCTCGGCACTGTGGTTGTTCCTGTGGCTTGGAAGATGCGTGGATACTTCGACTAT | |
| | TGGGGGCAACTCACTCTTGTTACCGTCTCAAGCGCTAGCACCAAAGGACCTAGTGTTTT | |
| | TCCTCTTGCCCCTCAAGTGCAACTCCTTGAAAGTGGAGGTGGCCTTGTCCAGCCAGGCG | |
| | GTTCACTGCGTTTGAGCTGTGCCGCATCCGGCTTTACCTTTTCCAGTTACACAATGAGT | |
| | TGGGTCCGTCAAGCACCCGGGCAATGTTTGGAATGGATGGGAACTATTTCATCTGGTGG | |
| | GACTTACACTTACTATCCCGATAGCGTCAAGGGACGTTTTACCATAAGCCGGGACAACG | |
| | CTAAAAATTCACTCTATCTTCAGATGAACAGTCTTAGGGCTGAGGATACCGCTGTTTAT | |
| | TATTGCGCCCGAGAAGCCATTTTCACCTACTGGGGGCGCGGTACACTTGTCACCGTTAG | |
| | CAGTGCTAGCACCAAAGGACCTAGTGTTTTTCCTCTTGCCCCTTCCTCAAAGTCTACCT | |
| | CTGGGGGGACAGCCGCTCTGGGCTGCCTGGTCAAGGATTATTTCCCAGAGCCTGTCACT | |
| | GTCAGTTGGAACTCTGGAGCCTTGACTTCTGGTGTTCATACATTTCCTGCTGTCCTTCA | |
| | AAGCAGCGGCTTGTACTCATTGTCTTCTGTTGTGACAGTACCCTCAAGCAGCCTCGGCA | |
| | CTCAGACATACATCTGCAATGTCAACCACAAACCCTCAAATACAAAGGTAGATAAAAAA | |
| | GTCGAACCAAAGTCTTGTGACAAAACTCACACGTGCCCACCGTGCCCAGCACCTGAACT | |
| | CCTGGGGGGACCGTCAGTCTTCCTCTTCCCCCCAAAACCCAAGGACACCCTCATGATCT | |
| | CCCGGACCCCTGAGGTCACATGCGTGGTGGTGGACGTGAGCCACGAAGACCCTGAGGTC | |
| | AAGTTCAACTGGTACGTGGACGGCGTGGAGGTGCATAATGCCAAGACAAAGCCGCGGGA | |
| | GGAGCAGTACAACAGCACGTACCGTGTGGTCAGCGTCCTCACCGTCCTGCACCAGGACT | |
| | GGCTGAATGGCAAGGAGTACAAGTGCAAGGTCTCCAACAAAGCCCTCCCAGCCCCCATC | |
| | GAGAAAACCATCTCCAAAGCCAAAGGGCAGCCCCGAGAACCACAGGTGTACACCCTGCC | |
| | CCCATGCCGGGAGGAGATGACCAAGAACCAGGTCAGCCTGTGGTGCCTGGTCAAAGGCT | |
| | TCTATCCCAGCGACATCGCCGTGGAGTGGGAGAGCAATGGGCAGCCGGAGAACAACTAC | |
| | AAGACCACGCCTCCCGTGCTGGACTCCGACGGCTCCTTCTTCCTCTACAGCAAGCTCAC | |
| | CGTGGACAAGAGCAGGTGGCAGCAGGGGAACGTCTTCTCATGCTCCGTGATGCATGAGG | |
| | CTCTGCACAACCACTACACGCAGAAGAGCCTCTCCCTGTCTCCGGGTAAA | |
| EPB11 LC | GACATACAGATGACTCAGAGCCCTGGTACACTGAGTGTATCTCCTGGGGAACGCGCCAC | 549 |
| | TCTGTCTTGCCGAGCCTCTCAAAGCGTTAGTTCCAACCTGGCTTGGTATCAGCAAAAAC | |
| | CCGGTCAAGCACCTCGCTTGCTTATCTACGGGGCCTCCACACGGGCAACCGGCATTCCT | |
| | GCACGCTTTAGTGGCTCTGGTAGCGGGACTGAATTCACTTTGACTATATCTTCCATGCA | |
| | GAGTGAGGATTTCGCCGTTTATTATTGTCAACAGTACAATAACTGGCCTCCACTCACTT | |
| | TTGGTGGTGGCACCAAAGTGGAGATAAAGCGTACGGTGGCAGCTCCCAGCGTTTTTATC | |
| | TTTCCCCCAGACATACAACTCACACAAAGTCCAAGCTCTCTTTCCCTTAGTCCAGGAGA | |
| | ACGCGTAACTCTCTCATGTAAAGCCTCCCAGGATATCAATAACTATCTCTCTTGGTACC | |
| | AACAAAAGCCTGACCAAGCTCCTAAGCTGCTTATCAAACGCGCCAATCGGTTGGTAGAC | |
| | GGAGTTCCCGACAGGTTCAGTGGGTCAGGCTCAGGTACAGATTTTACCTTGAAGATATC | |
| | ACGCGTGGAAGCCGAAGACGTAGGGGTATATTATTGTCTGAAATATGACGAGTTCCCCT | |
| | ATACTTTTGGGTGTGGAACTCGCCTGGAAATAAAACGTACGGTGGCAGCTCCCAGCGTT | |
| | TTTATCTTTCCCCCATCCGACGAGCAGCTCAAGAGTGGCACTGCCTCTGTAGTTTGTTT | |
| | GCTGAATAACTTCTATCCACGTGAAGCAAAAGTACAGTGGAAGGTCGATAATGCCCTTC | |
| | AGAGCGGTAACAGCCAAGAAAGTGTTACCGAGCAAGATTCCAAAGATTCCACTTACAGT | |
| | CTGTCCAGCACATTGACACTGAGTAAGGCTGATTACGAAAAACACAAGGTGTACGCATG | |
| | CGAGGTGACACACCAAGGTCTTTCATCTCCTGTAACTAAGAGCTTTAACCGGGGAGAAT | |
| | GTGGTGGTGGGGGCAGCGGGGGCGGAGGTAGTGGAGGCGGCGGTAGTGAACCAAAGAGT | |
| | AGTGACAAAACTCACACGTGCCCACCGTGCCCAGCACCTGAACTCCTGGGGGGACCGTC | |
| | AGTCTTCCTCTTCCCCCCAAAACCCAAGGACACCCTCATGATCTCCCGGACCCCTGAGG | |
| | TCACATGCGTGGTGGTGGACGTGAGCCACGAAGACCCTGAGGTCAAGTTCAACTGGTAC | |
| | GTGGACGGCGTGGAGGTGCATAATGCCAAGACAAAGCCGCGGGAGGAGCAGTACAACAG | |
| | CACGTACCGTGTGGTCAGCGTCCTCACCGTCCTGCACCAGGACTGGCTGAATGGCAAGG | |
| | AGTACAAGTGCAAGGTCTCCAACAAAGCCCTCCCAGCCCCCATCGAGAAAACCATCTCC | |
| | AAAGCCAAAGGGCAGCCCCGAGAACCACAGGTGTACACCCTGCCCCCATGCCGGGAGGA | |
| | GATGACCAAGAACCAGGTCAGCCTGTGGTGCCTGGTCAAAGGCTTCTATCCCAGCGACA | |
| | TCGCCGTGGAGTGGGAGAGCAATGGGCAGCCGGAGAACAACTACAAGACCACGCCTCCC | |
| | GTGCTGGACTCCGACGGCTCCTTCTTCCTCTACAGCAAGCTCACCGTGGACAAGAGCAG | |
| | GTGGCAGCAGGGGAACGTCTTCTCATGCTCCGTGATGCATGAGGCTCTGCACAACCACT | |
| | ACACGCAGAAGAGCCTCTCCCTGTCTCCGGGTAAA | |
| EPB12 HC | GAGGTCCAGCTGGTTGAGTCTGGAGGTGGTGTAGTCAGGCCAGGAGGTTCCCTGCGTCT | 550 |
| | GTCTTGCGCTGCCTCTGGGTTCACTGTGTCTGACTACAGTATGAACTGGGTTAGACAGG | |
| | CTCCAGGGAAAGGTTTGGAATGGATAGGGTTTATTCGCAATAAAGCCAATGCCTATACC | |
| | ACCGAATACAGCGCAAGCGTTAAAGGCAGGTTCACCATCAGTAGGGACGACTCTAAGAA | |
| | TACCCTCTACCTGCAGATGAATAGCTTGAAGACAGAGGATACCGCTGTTTATTACTGCA | |
| | CTACCTACCCCAGGTATCATGCTATGGACAGTTGGGGTCAAGGAACTATGGTCACCGTC | |
| | TCCTCTGCTAGCACCAAAGGACCTAGTGTTTTTCCTCTTGCCCCTCAAGTGCAACTCCT | |
| | TGAAAGTGGAGGTGGCCTTGTCCAGCCAGGCGGTTCACTGCGTTTGAGCTGTGCCGCAT | |
| | CCGGCTTTACCTTTTCCAGTTACACAATGAGTTGGGTCCGTCAAGCACCCGGGCAATGT | |
| | TTGGAATGGATGGGAACTATTTCATCTGGTGGGACTTACACTTACTATCCCGATAGCGT | |
| | CAAGGGACGTTTTACCATAAGCCGGGACAACGCTAAAAATTCACTCTATCTTCAGATGA | |
| | ACAGTCTTAGGGCTGAGGATACCGCTGTTTATTATTGCGCCCGAGAAGCCATTTTCACC | |
| | TACTGGGGGCGCGGTACACTTGTCACCGTTAGCAGTGCTAGCACCAAAGGACCTAGTGT | |
| | TTTTCCTCTTGCCCCTTCCTCAAAGTCTACCTCTGGGGGGACAGCCGCTCTGGGCTGCC | |
| | TGGTCAAGGATTATTTCCCAGAGCCTGTCACTGTCAGTTGGAACTCTGGAGCCTTGACT | |
| | TCTGGTGTTCATACATTTCCTGCTGTCCTTCAAAGCAGCGGCTTGTACTCATTGTCTTC | |
| | TGTTGTGACAGTACCCTCAAGCAGCCTCGGCACTCAGACATACATCTGCAATGTCAACC | |
| | ACAAACCCTCAAATACAAAGGTAGATAAAAAAGTCGAACCAAAGTCTTGTGACAAAACT | |
| | CACACGTGCCCACCGTGCCCAGCACCTGAACTCCTGGGGGGACCGTCAGTCTTCCTCTT | |
| | CCCCCCAAAACCCAAGGACACCCTCATGATCTCCCGGACCCCTGAGGTCACATGCGTGG | |
| | TGGTGGACGTGAGCCACGAAGACCCTGAGGTCAAGTTCAACTGGTACGTGGACGGCGTG | |
| | GAGGTGCATAATGCCAAGACAAAGCCGCGGGAGGAGCAGTACAACAGCACGTACCGTGT | |
| | GGTCAGCGTCCTCACCGTCCTGCACCAGGACTGGCTGAATGGCAAGGAGTACAAGTGCA | |
| | AGGTCTCCAACAAAGCCCTCCCAGCCCCCATCGAGAAAACCATCTCCAAAGCCAAAGGG | |
| | CAGCCCCGAGAACCACAGGTGTACACCCTGCCCCCATGCCGGGAGGAGATGACCAAGAA | |
| | CCAGGTCAGCCTGTGGTGCCTGGTCAAAGGCTTCTATCCCAGCGACATCGCCGTGGAGT | |
| | GGGAGAGCAATGGGCAGCCGGAGAACAACTACAAGACCACGCCTCCCGTGCTGGACTCC | |
| | GACGGCTCCTTCTTCCTCTACAGCAAGCTCACCGTGGACAAGAGCAGGTGGCAGCAGGG | |
| | GAACGTCTTCTCATGCTCCGTGATGCATGAGGCTCTGCACAACCACTACACGCAGAAGA | |
| | GCCTCTCCCTGTCTCCGGGTAAA | |
| EPB12 LC | GCCATACAGCTGACTCAATCACCTTCTTCTTTGAGCGCCTCTGTCGGTGATCGCGTTAC | 551 |
| | AATTACATGCCGAGCTTCTCAGTCTATCTCTAATAACCTCCACTGGTATCTCCAGAAAC | |
| | CTGGACAGTCACCACAGCTTCTGATATATTACGGCTTTCAGTCTATAAGTGGAGTCCCA | |
| | AGTAGATTTTCCGGTAGTGGATCTGGCACCGATTTTACTCTTACTATCTCTAGCCTCCA | |
| | ACCTGAAGATTTCGCAACCTATTACTGCCAGCAGGCCAATTCCTGGCCCTTGACCTTCG | |
| | GAGGTGGGACTAAGCTGGAAATCAAACGTACGGTGGCAGCTCCCAGCGTTTTTATCTTT | |
| | CCCCCAGACATACAACTCACACAAAGTCCAAGCTCTCTTTCCCTTAGTCCAGGAGAACG | |
| | CGTAACTCTCTCATGTAAAGCCTCCCAGGATATCAATAACTATCTCTCTTGGTACCAAC | |
| | AAAAGCCTGACCAAGCTCCTAAGCTGCTTATCAAACGCGCCAATCGGTTGGTAGACGGA | |
| | GTTCCCGACAGGTTCAGTGGGTCAGGCTCAGGTACAGATTTTACCTTGAAGATATCACG | |
| | CGTGGAAGCCGAAGACGTAGGGGTATATTATTGTCTGAAATATGACGAGTTCCCCTATA | |
| | CTTTTGGGTGTGGAACTCGCCTGGAAATAAAACGTACGGTGGCAGCTCCCAGCGTTTTT | |
| | ATCTTTCCCCCATCCGACGAGCAGCTCAAGAGTGGCACTGCCTCTGTAGTTTGTTTGCT | |
| | GAATAACTTCTATCCACGTGAAGCAAAAGTACAGTGGAAGGTCGATAATGCCCTTCAGA | |
| | GCGGTAACAGCCAAGAAAGTGTTACCGAGCAAGATTCCAAAGATTCCACTTACAGTCTG | |
| | TCCAGCACATTGACACTGAGTAAGGCTGATTACGAAAAACACAAGGTGTACGCATGCGA | |
| | GGTGACACACCAAGGTCTTTCATCTCCTGTAACTAAGAGCTTTAACCGGGGAGAATGTG | |
| | GTGGTGGGGGCAGCGGGGGCGGAGGTAGTGGAGGCGGCGGTAGTGAACCAAAGAGTAGT | |
| | GACAAAACTCACACGTGCCCACCGTGCCCAGCACCTGAACTCCTGGGGGGACCGTCAGT | |
| | CTTCCTCTTCCCCCCAAAACCCAAGGACACCCTCATGATCTCCCGGACCCCTGAGGTCA | |
| | CATGCGTGGTGGTGGACGTGAGCCACGAAGACCCTGAGGTCAAGTTCAACTGGTACGTG | |
| | GACGGCGTGGAGGTGCATAATGCCAAGACAAAGCCGCGGGAGGAGCAGTACAACAGCAC | |
| | GTACCGTGTGGTCAGCGTCCTCACCGTCCTGCACCAGGACTGGCTGAATGGCAAGGAGT | |
| | ACAAGTGCAAGGTCTCCAACAAAGCCCTCCCAGCCCCCATCGAGAAAACCATCTCCAAA | |
| | GCCAAAGGGCAGCCCCGAGAACCACAGGTGTACACCCTGCCCCCATGCCGGGAGGAGAT | |
| | GACCAAGAACCAGGTCAGCCTGTGGTGCCTGGTCAAAGGCTTCTATCCCAGCGACATCG | |
| | CCGTGGAGTGGGAGAGCAATGGGCAGCCGGAGAACAACTACAAGACCACGCCTCCCGTG | |
| | CTGGACTCCGACGGCTCCTTCTTCCTCTACAGCAAGCTCACCGTGGACAAGAGCAGGTG | |
| | GCAGCAGGGGAACGTCTTCTCATGCTCCGTGATGCATGAGGCTCTGCACAACCACTACA | |
| | CGCAGAAGAGCCTCTCCCTGTCTCCGGGTAAA | |

Table 52 below shows the polypeptide sequences of the heavy chain variable region (VH) and light chain variable region (VL) of the engineered antibodies targeting EphA2. Table 53 below shows the nucleotide sequences of the heavy chain variable region (VH) and light chain variable region (VL) of the engineered antibodies targeting EphA2.

TABLE 52

| | | | SEQ ID |
|---|---|---|---|
| Name | | Sequence | NO |
| EPB01 | VH1 | EVQLLESGGGLVQPGGSLRLSCAASGFTFSRYQMMWVRQAPGKGLEWVSSISPSGGVTLYA | 135 |
| | | DSVKGRFTISRDNSKNTLYLQMNSLRAEDTAVYYCTRELLGTVVVPVAWKMRGYFDYWGQL | |
| | | TLVTVSS | |
| | VH2 | EVQLLESGGGLVQPGGSLRLSCAASGFTFSHYMMAWVRQAPGKCLEWVSRIGPSGGPTHYA | 136 |
| | | DSVKGRFTISRDNSKNTLYLQMNSLRAEDTAVYYCAGYDSGYDYVAVAGPAEYFQHWGQGT | |
| | | LVTVSS | |
| | VL1 | DIQMTQSPGTLSVSPGERATLSCRASQSVSSNLAWYQQKPGQAPRLLIYGASTRATGIPAR | 137 |
| | | FSGSGSGTEFTLTISSMQSEDFAVYYCQQYNNWPPLTFGGGTKVEIK | |
| | VL2 | DIQMTQSPSSLSASVGDRVTITCRASQSISTWLAWYQQKPGKAPKLLIYKASNLHTGVPSR | 138 |
| | | FSGSGSGTEFSLTISGLQPDDFATYYCQQYNSYSRTFGCGTKVEIK | |
| EPB02 | VH1 | EVQLVESGGGVVRPGGSLRLSCAASGFTVSDYSMNWVRQAPGKGLEWIGFIRNKANAYTTE | 130 |
| | | YSASVKGRFTISRDDSKNTLYLQMNSLKTEDTAVYYCTTYPRYHAMDSWGQGTMVTVSS | |
| | VH2 | EVQLLESGGGLVQPGGSLRLSCAASGFTFSHYMMAWVRQAPGKCLEWVSRIGPSGGPTHYA | 136 |
| | | DSVKGRFTISRDNSKNTLYLQMNSLRAEDTAVYYCAGYDSGYDYVAVAGPAEYFQHWGQGT | |
| | | LVTVSS | |
| | VL1 | AIQLTQSPSSLSASVGDRVTITCRASQSISNNLHWYLQKPGQSPQLLIYYGFQSISGVPSR | 140 |
| | | FSGSGSGTDFTLTISSLQPEDFATYYCQQANSWPLTFGGGTKLEIK | |
| | VL2 | DIQMTQSPSSLSASVGDRVTITCRASQSISTWLAWYQQKPGKAPKLLIYKASNLHTGVPSR | 138 |
| | | FSGSGSGTEFSLTISGLQPDDFATYYCQQYNSYSRTFGCGTKVEIK | |
| EPB03 | VH1 | QVQLLESGGGLVQPGGSLRLSCAASGFTFSSYTMSWVRQAPGQALEWMGTISSGGTYTYYP | 141 |
| | | DSVKGRFTISRDNAKNSLYQMNSLRAEDTAVYYCAREAIFTYWGRGTLVTVSS | |
| | VH2 | EVQLLESGGGLVQPGGSLRLSCAASGFTFSHYMMAWVRQAPGKCLEWVSRIGPSGGPTHYA | 136 |
| | | DSVKGRFTISRDNSKNTLYLQMNSLRAEDTAVYYCAGYDSGYDYVAVAGPAEYFQHWGQGT | |
| | | LVTVSS | |
| | VL1 | DIQLTQSPSSLSLSPGERVTLSCKASQDINNYLSWYQQKPDQAPKLLIKRANRLVDGVPDR | 142 |
| | | FSGSGSGTDFTLKISRVEAEDVGVYYCLKYDEFPYTFGQGTRLEIK | |
| | VL2 | DIQMTQSPSSLSASVGDRVTITCRASQSISTWLAWYQQKPGKAPKLLIYKASNLHTGVPSR | 138 |
| | | FSGSGSGTEFSLTISGLQPDDFATYYCQQYNSYSRTFGCGTKVEIK | |
| EPB04 | VH1 | EVQLLESGGGLVQPGGSLRLSCAASGFTFSHYMMAWVRQAPGKGLEWVSRIGPSGGPTHYA | 143 |
| | | DSVKGRFTISRDNSKNTLYLQMNSLRAEDTAVYYCAGYDSGYDYVAVAGPAEYFQHWGQGT | |
| | | LVTVSS | |
| | VH2 | EVQLLESGGGLVQPGGSLRLSCAASGFTFSRYQMMWYRQAPGKCLEWVSSISPSGGVTLYA | 144 |
| | | DSVKGRFTISRDNSKNTLYLQMNSLRAEDTAVYYCTRELLGTVVVPVAWKMRGYFDYNGQL | |
| | | TLVTVSS | |
| | VL1 | DIQMTQSPSSLSASVGDRVTITCRASQSISTWLAWYQQKPGKAPKLLIYKASNLHTGVPSR | 145 |
| | | FSGSGSGTEFSLTISGLQPDDFATYYCQQYNSYSRTFGQGTKVEIK | |
| | VL2 | DIQMTQSPGTLSVSPGERATLSCRASQSVSSNLAWYQQKPDQAPRLLIYGASTRATGIPAR | 146 |
| | | FSGSGSGTEFTLTISSMQSEDFAVYYCQQYNNWPPLTFGCGTKVEIK | |
| EPB06 | VH1 | EVQLLESGGGLVQPGGSLRLSCAASGFTFSHYMMAWVRQAPGKGLEWVSRIGPSGGPTHYA | 143 |
| | | DSVKGRFTISRDNSKNTLYLQMNSLRAEDTAVYYCAGYDSGYDYVAVAGPAEYFQHWGQGT | |
| | | LVTVSS | |
| | VH2 | EVQLVESGGGVVRPGGSLRLSCAASGFTVSDYSMNWVRQAPGKCLEWIGFIRNKANAYTTE | 147 |
| | | YSASVKGRFTISRDDSKNTLYLQMNSLKTEDTAVYYCTTYPRYHAMDSWGQGTMYTVSS | |
| | VL1 | DIQMTQSPSSLSASVGDRVTITCRASQSISTWLAWYQQKPGKAPKLLIYKASNLHTGVPSR | 145 |
| | | FSGSGSGTEFSLTISGLQPDDFATYYCQQYNSYSRTFGQGTKVEIK | |
| | VL2 | AIQLTQSPSSLSASVGDRYTITCRASQSISNNLHWYLQKPGQSPQLLIYYGFQSISGVPSR | 148 |
| | | FSGSGSGTDFTLTISSLQPEDFATYYCQQANSWPLTFGCGTKLEIK | |
| EPB06 | VH1 | EVQLLESGGGLVQPGGSLRLSCAASGFTFSHYMMAWVRQAPGKGLEWVSRIGPSGGPTHYA | 143 |
| | | DSVKGRFTISRDNSKNTLYLQMNSLRAEDTAVYYCAGYDSGYDYVAVAGPAEYFQHWGQGT | |
| | | LVTVSS | |
| | VH2 | QVQLLESGGGLYQPGGSLRLSCAASGFTFSSYTMSWYRQAPGQCLEWMGTISSGGTYTYYP | 149 |
| | | DSVKGRFTISRDNAKNSLYLQMNSLRAEDTAVYYCAREAIFTYWGRGTLVTVSS | |
| | VL1 | DIQMTQSPSSLSASVGDRVTITCRASQSISTWLAWYQQKPGKAPKLLIYKASNLHTGVPSR | 145 |
| | | FSGSGSGTEFSLTISGLQPDDFATYYCQQYNSYSRTFGQGTKVEIK | |
| | VL2 | DIQLTQSPSSLSLSPGERVTLSCKASQDINNYLSWYQQKPDQAPKLLIKRANRLVDGVPDR | 150 |
| | | FSGSGSGTDFTLKISRVEARDVGVYYCLKYDEFPYTFGCGTRLEIK | |
| EPB07 | VH1 | EVQLVESGGGVVRPGGSLRSLSCAASGTFVSDYSMNWVRQAPGLGLEWIGFIRNKANATTTE | 139 |
| | | YSASVKGRFTISRDDSKNTLYLQMNSLKTEDTAVYYCTTYPKYHAMDSWGQGTMVTVSS | |
| | VH2 | EVQLLESGGGLVQPGGSLRLSCAASGFTFSRYQMMWYRQAPGKCLEWVSSISPSGGVTLYA | 144 |
| | | DSVKGRFTISRDNSKNTLYLQMNSLRAEDTAVYYCTRELLGTVVVPVAWKMRGYFDYNGQL | |
| | | TLVTVSS | |
| | VL1 | AIQLTQSPSSLSASVGDRVTITCRASQSISNNLHWYLQKPGQSPQLLIYYGFQSISGVPSR | 140 |
| | | FSGSGSGTDFTLTISSLQPEDFATYYCQQANSWPLTFGGGTKLEIK | |
| | VL3 | DIQMTQSPGTLSVSPGERATLSCRASQSVSSNLAWYQQKPDQAPRLLIYGASTRATGIPAR | 146 |
| | | FSGSGSGTEFTLTISSMQSEDFAVYYCQQYNNWPPLTFGCGTKVEIK | |
| EPB08 | VH1 | QVQLLESGGGLVQPGGSLRLSCAASGFTFSSYTMSWVRQAPGQALEWMGTISSGGTYTYYP | 141 |
| | | DSVKGRFTISRDNAKNSLYQMNSLRAEDTAVYYCAREAIFTYWGRGTLVTVSS | |
| | VH2 | EVQLLESGGGLVQPGGSLRLSCAASGFTFSRYQMMWYRQAPGKCLEWVSSISPSGGVTLYA | 144 |
| | | DSVKGRFTISRDNSKNTLYLQMNSLRAEDTAVYYCTRELLGTVVVPVAWKMRGYFDYNGQL | |
| | | TLVTVSS | |
| | VL1 | DIQLTQSPSSLSLSPGERVTLSCKASQDINNYLSWYQQKPDQAPKLLIKRANRLVDGVPDR | 142 |
| | | FSGSGSGTDFTLKISRVEAEDVGVYYCLKYDEFPYTFGQGTRLEIK | |
| | VL2 | DIQMTQSPGTLSVSPGERATLSCRASQSVSSNLAWYQQKPDQAPRLLIYGASTRATGIPAR | 146 |
| | | FSGSGSGTEFTLTISSMQSEDFAVYYCQQYNNWPPLTFGCGTKVEIK | |
| EPB09 | VH1 | EVQLLESGGGLVQPGGSLRLSCAASGFTFSRYQMMWVRQAPGKGLEWVSSISPSGGVTLYA | 135 |
| | | DSVKGRFTISRDNSKNTLYLQMNSLRAEDTAVYYCTRELLGTVVVPVAWKMRGYFDYWGQL | |
| | | TLVTVSS | |
| | VH2 | EVQLVESGGGVVRPGGSERLSCAASGFTVSDYSMNWVRQAPGKCLEWIGFIRNKANAYTTE | 147 |
| | | YSASVKGRFTISRDDSKNTLYLQMNSLKTEDTAVYYCTTYPRYHAMDSWGQGTMVTVSS | |
| | | DIQMTQSPGTLSVSPGERATLSCRASQSVSSNLAWYQQKPGQAPRLLIYGASTRATGIPAR | 137 |
| | | FSGSGSGTEFTLTISSMQSEDFAVYYCQQYNNWPPLTFGGGTKVEIK | |
| | VL2 | AIQLTQSPSSLSASVGDRYTITCRASQSISNNLHWYLQKPGQSPQLLIYYGFQSISGVPSR | 148 |
| | | FSGSGSGTDFTLTISSLQPEDFATYYCQQANSWPLTFGCGTKLEIK | |
| EPB10 | VH1 | QVQLLESGGGLVQPGGSLRLSCAASGFTFSSYTMSWVRQAPGQALEWMGTISSGGTYTYYP | 141 |
| | | DSVKGRFTISRDNAKNSLYQMNSLRAEDTAVYYCAREAIFTYWGRGTLVTVSS | |
| | VH2 | EVQLVESGGGVVRPGGSLRLSCAASGFTVSDYSMNWVRQAPGKCLEWIGFIRNKANAYTTE | 147 |
| | | YSASVKGRFTISKDDSKNTLYLQMNSLKTEDTAVYYCTTYPRYHAMDSWGQGTMVTVSS | |
| | VL1 | DIQLTQSPSSLSLSPGERVTLSCKASQDINNYLSWYQQKPDQAPKLLIKRANRLVDGVPDR | 142 |
| | | FSGSGSGTDFTLKISRVEAEDVGVYYCLKYDEFPYTFGQGTRLEIK | |
| | VL2 | AIQLTQSPSSLSASVGDRVTITCRASQSISNNLHWYLQKPGQSPQLLIYYGFQSISGVPSR | 148 |
| | | FSGSGSGTDFTLTISSLQPEDFATYYCQQANSWPLTFGCGTKLEIK | |
| EPB11 | VH1 | EVQLLESGGGLVQPGGSLRLSCAASGFTFSRYQMMWVRQAPGKGLEWVSSISPSGGVTLYA | 135 |
| | | DSVKGRFTISRDNSKNTLYLQMNSLRAEDTAVYYCTRELLGTVVVPVAWKMRGYFDYWGQL | |
| | | TLVTVSS | |
| | VH2 | QVQLLESGGGLYQPGGSLRLSCAASGFTFSSYTMSWYRQAPGQCLEWMGTISSGGTYTYYP | 149 |
| | | DSVKGRFTISRDNAKNSLYLQMNSLRAEDTAVYYCAREAIFTYWGRGTLVTVSS | |
| | VL1 | DIQMTQSPGTLSVSPGERATLSCRASQSVSSNLAWYQQKPGQAPRLLIYGASTRATGIPAR | 137 |
| | | FSGSGSGTEFTLTISSMQSEDFAVYYCQQYNNWPPLTFGGGTKVEIK | |
| | VL2 | DIQLTQSPSSLSLSPGERVTLSCKASQDINNYLSWYQQKPDQAPKLLIKRANRLVDGVPDR | 150 |
| | | FSGSGSGTDFTLKISRVEARDVGVYYCLKYDEFPYTFGCGTRLEIK | |
| EPB12 | VH1 | EVQLVESGGGVVRPGGSLRLSCAASGFTVSDYSMNWVRQAPGKGLEWIGFIRNKANAYTTE | 130 |
| | | YSASVKGRFTISRDDSKNTLYLQMNSLKTEDTAVYYCTTYPRYHAMDSWGQGTMVTVSS | |
| | VH2 | QVQLLESGGGLYQPGGSLRLSCAASGFTFSSYTMSWYRQAPGQCLEWMGTISSGGTYTYYP | 149 |
| | | DSVKGRFTISRDNAKNSLYLQMNSLRAEDTAVYYCAREAIFTYWGRGTLVTVSS | |
| | VL1 | AIQLTQSPSSLSASVGDRVTITCRASQSISNNLHWYLQKPGQSPQLLIYYGFQSISGVPSR | 140 |
| | | FSGSGSGTDFTLTISSLQPEDFATYYCQQANSWPLTFGGGTKLEIK | |
| | VL2 | DIQLTQSPSSLSLSPGERVTLSCKASQDINNYLSWYQQKPDQAPKLLIKRANRLVDGVPDR | 150 |
| | | FSGSGSGTDFTLKISRVEARDVGVYYCLKYDEFPYTFGCGTRLEIK | |

TABLE 53

| | | | SEQ |
|---|---|---|---|
| | | | ID |
| Name | | Sequence | NO |
| EPB01 | VH1 | GAGGTGCAACTGCTTGAGTCAGGAGGAGGACTGGTTCAACCCGGGGGGAGCCTTCGACTCT | 552 |
| | | CATGTGCAGCCTCTGGGTTTACATTTAGTCGTTACCAAATGATGTGGGTACGCCAGGCCCC | |
| | | CGGAAAAGGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTCTTGTTACCGTCTCAAGC | |
| | VH2 | GAGGTTCAACTCCTTGAATCCGGAGGAGGACTTGTCCAACCAGGCGGCAGCCTTAGGCTGT | 553 |
| | | CCTGCGCTGCCTCAGGCTTTACATTCAGTCATTACATGATGGCCTGGGTTAGACAGGCACC | |
| | | CGGCAAATGTCTGGAATGGGTCAGCCGAATAGGACCATCAGGAGGTCCCACTCACTATGCC | |
| | | GATTCTGTAAAAGGGAGGTTTACAATTTCCAGAGACAATTCAAAGAATACCCTTTACCTCC | |
| | | AGATGAACTCATTGAGAGCCGAGGACACAGCCGTATATTATTGCGCAGGTTATGATTCCGG | |
| | | TTACGATTACGTTGCAGTCGCAGGCCCAGCCGAATACTTCCAACATTGGGGTCAGGGAACC | |
| | | CTCGTGACCGTGTCCAGT | |
| | VL1 | GACATACAGATGACTCAGAGCCCTGGTACACTGAGTGTATCTCCTGGGGAACGCGCCACTC | 554 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCTCCACTCACTTTTGGTGGTGG | |
| | | CACCAAAGTGGAGATAAAG | |
| | VL2 | GACATACAGATGACACAAAGTCCAAGTAGCCTTTCTGCATCCGTTGGAGACCGCGTAACCA | 556 |
| | | TAACTTGTCGAGCATCACAAAGCATAAGCACTTGGCTTGCTTGGTATCAGCAGAAGCCAGG | |
| | | CAAAGCACCCAAGCTGCTGATTTATAAAGCCTCTAATCTCCATACAGGAGTTCCCAGCCGT | |
| | | TTCTCAGGCAGCGGGTCTGGAACCGAATTCTCTCTGACCATCTCTGGCTTGCAACCTGATG | |
| | | ACTTTGCAACATATTACTGTCAACAATATAATTCATATAGTCGTACTTTCGGGTGTGGCAC | |
| | | TAAGGTGGAAATCAAA | |
| EPB02 | VH1 | GAGGTCCAGCTGGTTGAGTCTGGAGGTGGTGTAGTCAGGCCAGGAGGTTCCCTGCGTCTGT | 556 |
| | | CTTGCGCTGCCTCTGGGTTCACTGTGTCTGACTACAGTATGAACTGGGTTAGACAGGCTCC | |
| | | AGGGAAAGGTTTGGAATGGATAGGGTTTATTCGCAATAAAGCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGGGTCAAGGAACTATGGTCACCGTCTCCTCT | |
| | VH2 | GAGGTTCAACTCCTTGAATCCGGAGGAGGACTTGTCCAACCAGGGGGCAGCCTTAGGCTGT | 553 |
| | | CCTGCGCTGCCTCAGGCTTTACATTCAGTCATTACATGATGGCCTGGGTTAGACAGGCACC | |
| | | CGGCAAATGTCTGGAATGGGTCAGCCGAATAGGACCATCAGGAGGTCCCACTCACTATGCC | |
| | | GATTCTGTAAAAGGGAGGTTTACAATTTCCAGAGACAATTCAAAGAATACCCTTTACCTCC | |
| | | AGATGAACTCATTGAGAGCCGAGGACACAGCCGTATATTATTGCGCAGGTTATGATTCCGG | |
| | | TTACGATTACGTTGCAGTCGCAGGCCCAGCCGAATACTTCCAACATTGGGGTCAGGGAACC | |
| | | CTCGTGACCGTGTCCAGT | |
| | VL1 | GCCATACAGCTGACTCAATCACCTTCTTCTTTGAGCGCCTCTGTCGGTGATCGCGTTACAA | 557 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGAGGTGGGAC | |
| | | TAAGCTGGAAATCAAA | |
| | VL2 | GACATACAGATGACACAAAGTCCAAGTAGCCTTTCTGCATCCGTTGGAGACCGCGTAACCA | 556 |
| | | TAACTTGTCGAGCATCACAAAGCATAAGCACTTGGCTTGCTTGGTATCAGCAGAAGCCAGG | |
| | | CAAAGCACCCAAGCTGCTGATTTATAAAGCCTCTAATCTCCATACAGGAGTTCCCAGCCGT | |
| | | TTCTCAGGCAGCGGGTCTGGAACCGAATTCTCTCTGACCATCTCTGGCTTGCAACCTGATG | |
| | | ACTTTGCAACATATTACTGTCAACAATATAATTCATATAGTCGTACTTTCGGGTGTGGCAC | |
| | | TAAGGTGGAAATCAAA | |
| EPB03 | VH1 | CAAGTGCAACTCCTTGAAAGTGGAGGTGGCCTTGTCCAGCCAGGCGGTTCACTGCGTTTGA | 558 |
| | | GCTGTGCCGCATCCGGCTTTACCTTTTCCAGTTACACAATGAGTTGGGTCCGTCAAGCACC | |
| | | CGGGCAAGCTTTGGAATGGATGGGAACTATTTCATCTGGTGGGACTTACACTTACTATCCC | |
| | | GATAGCGTCAAGGGACGTTTTACCATAAGCCGGGACAACGCTAAAAATTCACTĆTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VH2 | GAGGTTCAACTCCTTGAATCCGGAGGAGGACTTGTCCAACCAGGCGGCAGCCTTAGGCTGT | 553 |
| | | CCTGCGCTGCCTCAGGCTTTACATTCAGTCATTACATGATGGCCTGGGTTAGACAGGCACC | |
| | | CGGCAAATGTCTGGAATGGGTCAGCCGAATAGGACCATCAGGAGGTCCCACTCACTATGCC | |
| | | GATTCTGTAAAAGGGAGGTTTACAATTTCCAGAGACAATTCAAAGAATACCCTTTACCTCC | |
| | | AGATGAACTCATTGAGAGCCGAGGACACAGCCGTATATTATTGCGCAGGTTATGATTCCGG | |
| | | TTACGATTACGTTGCAGTCGCAGGCCCAGCCGAATACTTCCAACATTGGGGTCAGGGAACC | |
| | | CTCGTGACCGTGTCCAGT | |
| | VL1 | GACATACAACTCACACAAAGTCCAAGCTCTCTTTCCCTTAGTCCAGGAGAACGCGTAACTC | 559 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTCGGTCAGCCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGCAAGGAAC | |
| | | TCGCCTGGAAATAAAA | |
| | VL2 | GACATACAGATGACACAAAGTCCAAGTAGCCTTTCTGCATCCGTTGGAGACCGCGTAACCA | 556 |
| | | TAACTTGTCGAGCATCACAAAGCATAAGCACTTGGCTTGCTTGGTATCAGCAGAAGCCAGG | |
| | | CAAAGCACCCAAGCTGCTGATTTATAAAGCCTCTAATCTCCATACAGGAGTTCCCAGCCGT | |
| | | TTCTCAGGCAGCGGGTCTGGAACCGAATTCTCTCTGACCATCTCTGGCTTGCAACCTGATG | |
| | | ACTTTGCAACATATTACTGTCAACAATATAATTCATATAGTCGTACTTTCGGGTGTGGCAC | |
| | | TAAGGTGGAAATCAAA | |
| EPB04 | VH1 | GAGGTACAGTTGCTGGAGTCAGGAGGTGGATTGGTCCAACCCGGAGGATCTCTTCGTCTGT | 450 |
| | | CCTGCGCCGCCTCAGGATTTACCTTCTCTCATTATATGATGGCATGGGTACGTCAGGCTCC | |
| | | AGGCAAAGGTCTGGAATGGGTTAGTCGGATTGGTCCCTCAGGGGGTCCTACCCATTATGCC | |
| | | GATTCTGTAAAGGGCCGTTTTACCATAAGCAGAGACAACTCTAAGAACACCCTTTACCTTC | |
| | | AGATGAATAGCCTGAGGGCTGAGGATACCGCAGTGTATTACTGCGCAGGCTATGACTCTGG | |
| | | GTACGACTATGTCGCCGTAGCAGGACCTGCCGAGTATTTTCAACACTGGGGACAGGGGACC | |
| | | CTTGTCACAGTTTCTAGT | |
| | VH2 | GAGGTGCAACTGCTTGAGTCAGGAGGAGGACTGGTTCAACCCGGCGGGAGCCTTCGACTCT | 560 |
| | | CATGTGCAGCCTCTGGGTTTACATTTAGTCGTTACCAAATGATGTGGGTACGCCAGGCCCC | |
| | | CGGAAAATGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTCTTGTTACCGTCTCAAGC | |
| | VL1 | GATATTCAAATGACACAAAGCCCAAGTTCCTTGTCCGCCTCAGTTGGTGATCGTGTGACAA | 451 |
| | | TAACCTGTCGGGCTTCACAATCCATATCTACATGGCTGGCTTGGTACCAGCAAAAGCCAGG | |
| | | TAAAGCCCCAAAACTCCTGATTTACAAGGCAAGTAACTTGCATACTGGGGTACCCAGCCGT | |
| | | TTCTCTGGGTCAGGCTCTGGGACAGAGTTTAGTCTTACAATTTCTGGTCTGCAACCCGATG | |
| | | ACTTCGCTACCTATTACTGTCAACAATATAATAGTTATTCTCGAACATTTGGTCAGGGAAC | |
| | | AAAAGTGGAAATCAAA | |
| | VL2 | GACATACAGATGACTCAGAGCCCTGGTACACTGAGTGTATCTCCTGGGGAACGCGCCACTC | 561 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCTCCACTCACTTTTGGTTGTGG | |
| | | CACCAAAGTGGAGATAAAG | |
| EPB05 | VH1 | GAGGTACAGTTGCTGGAGTCAGGAGGTGGATTGGTCCAACCCGGAGGATCTCTTCGTCTGT | 450 |
| | | CCTGCGCCGCCTCAGGATTTACCTTCTCTCATTATATGATGGCATGGGTACGTCAGGCTCC | |
| | | AGGCAAAGGTCTGGAATGGGTTAGTCGGATTGGTCCCTCAGGGGGTCCTACCCATTATGCC | |
| | | GATTCTGTAAAGGGCCGTTTTACCATAAGCAGAGACAACTCTAAGAACACCCTTTACCTTC | |
| | | AGATGAATAGCCTGAGGGCTGAGGATACCGCAGTGTATTACTGCGCAGGCTATGACTCTGG | |
| | | GTACGACTATGTCGCCGTAGCAGGACCTGCCGAGTATTTTCAACACTGGGGACAGGGGACC | |
| | | CTTGTCACAGTTTCTAGT | |
| | VH2 | GAGGTCCAGCTGGTTGAGTCTGGAGGTGGTGTAGTCAGGCCAGGAGGTTCCCTGCGTCTGT | 562 |
| | | CTTGCGCTGCCTCTGGGTTCACTGTGTCTGACTACAGTATGAACTCGGTTAGACAGGCTCC | |
| | | AGGGAAATGTTTGGAATGGATAGGGTTTATTCGCAATAAAGCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGGGTCAAGGAACTATGGTCACCGTCTCCTCT | |
| | VL1 | GATATTCAAATGACACAAAGCCCAAGTTCCTTGTCCGCCTCAGTTGGTGATCGTGTGACAA | 461 |
| | | TAACCTGTCGGGCTTCACAATCCATATCTACATGGCTGGCTTGGTACCAGCAAAAGCCAGG | |
| | | TAAAGCCCCAAAACTCCTGATTTACAAGGCAAGTAACTTGCATACTGGGGTACCCAGCCGT | |
| | | TTCTCTGGGTCAGGCTCTGGGACAGAGTTTAGTCTTACAATTTCTGGTCTGCAACCCGATG | |
| | | ACTTCGCTACCTATTACTGTCAACAATATAATAGTTATTCTCGAACATTTGGTCAGGGAAC | |
| | | AAAAGTGGAAATCAAA | |
| | VL2 | GCCATACAGCTGACTCAATCACCTTCTTCTTTGAGCGCCTCTGTCGGTGATCGCGTTACAA | 563 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGATGTGGGAC | |
| | | TAAGCTGGAAATCAAA | |
| EPB06 | VH1 | GAGGTACAGTTGCTGGAGTCAGGAGGTGGATTGGTCCAACCCGGAGGATCTCTTCGTCTGT | 450 |
| | | CCTGCGCCGCCTCAGGATTTACCTTCTCTCATTATATGATGGCATGGGTACGTCAGGCTCC | |
| | | AGGCAÀAGGTCTGGAATGGGTTAGTCGGATTGGTCCCTCAGGGGGTCCTACCCATTATGCC | |
| | | GATTCTGTAAAGGGCCGTTTTACCATAAGCAGAGACAACTCTAAGAACACCCTTTACCTTC | |
| | | AGATGAATAGCCTGAGGCCTGAGGATACCGCAGTGTATTACTGCGCAGGCTATGACTCTGG | |
| | | GTACGACTATGTCGCCGTAGCAGGACCTGCCGAGTATTTCAACACTGGGGACAGGGGACC | |
| | | CTTGTCACAGTTTCTAGT | |
| | VH2 | CAAGTGCAACTCCTTGAAAGTGGAGGTGGCCTTGTCCAGCCAGGCGGTTCACTGCGTTTGA | 564 |
| | | GCTGTGCCGCATCCGGCTTTACCTTTTCCAGTTACACAATGAGTTGGGTCCGTCAAGCACC | |
| | | CGGGCAATGTTTGGAATGGATGGGAACTATTTCATCTGGTGGGACTTACACTTACTATCCC | |
| | | GATAGCGTCAAGGGACGTTTTACCATAAGCCGGGACAACGCTAAAAATTCACTCTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VL1 | GATATTCAAATGACACAAAGCCCAAGTTCCTTGTCCGCCTCAGTTGGTGATCGTGTGACAA | 451 |
| | | TAACCTGTCGGGCTTCACAATCCATATCTACATGGCTGGCTTGGTACCAGCAAAAGCCAGG | |
| | | TAAAGCCCCAAAACTCCTGATTTACAAGGCAAGTAACTTGCATACTGGGGTACCCAGCCGT | |
| | | TTCTCTGGGTCAGGCTCTGGGACAGAGTTTAGTCTTACAATTTCTGGTCTGCAACCCGATG | |
| | | ACTTCGCTACCTATTACTGTCAACAATATAATAGTTATTCTCGAACATTTGGTCAGGGAAC | |
| | | AAAAGTGGAAATCAAA | |
| | VL2 | GACATACAACTCACACAAAGTCCAAGCTCTCTTTCCCTTAGTCCAGGAGAACGCGTAACTC | 565 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTGGGTCAGGCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGTGTGGAAC | |
| | | TCGCCTGGAAATAAAA | |
| EPB07 | VH1 | GAGGTCCAGCTGGTTGAGTCTGGAGGTGGTGTAGTCAGGCCAGGAGGTTCCCTGCGTCTGT | 556 |
| | | CTTGCGCTGCCTCTGGGTTCACTGTGTCTGACTACAGTATGAACTGGGTTAGACAGGCTCC | |
| | | AGGGAAAGGTTTGGAATGGATAGGGTTTATTCGCAATAAACCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGGGTCAAGGAACTATGGTCACCGTCTCCTCT | |
| | VH2 | GAGGTGCAACTGCTTGAGTCAGGAGGAGGACTGGTTCAAACCGGGGGGAGCCTTCGACTCT | 560 |
| | | CATGTGCAGCCTCTGGGTTTACATTTAGTCGTTACCAAATGATGTGGGTACGCCAGGCCCC | |
| | | CGGAAAATGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTTCTTGTTACCGTCTCAAGC: | |
| | VL1 | GCCATACAGCTGACTCAATCACCTTCTTCTTTGAGCGCCTCTGTCGGTGATCGCGTTACAA | 577 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGAGGTGGGAC | |
| | | TAAGCTGGAAATCAAA | |
| | VL2 | GACATACAGATGACTCAGAGCCCTGGTACACTGAGTGTATCTCCTGGGGAACGCGCCACTC | 561 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCTCCACTCACTTTTGGTTGTGG | |
| | | CACCAAAGTGGAGATAAAG | |
| EPB08 | VH1 | CAAGTGCAACTCCTTGAAAGTGGAGGTGGCCTTGTCCAGCCAGGCGGTTCACTGCGTTTGA | 558 |
| | | GCTGTGCCGCATCCGGCTTTACCTTTTCCAGTTACACAATGAGTTGGGTCCGTCAAGCACC | |
| | | CGGGCAAGCTTTGGAATGGATGGGAACTATTTCATCTGGTGGGACTTACACTTACTATCCC | |
| | | GATAGCGTCAAGGGACGTTTTACCATAAGCCGGGACAACGCTAAAAATTCACTCTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VH2 | GAGGTGCAACTGCTTGAGTCAGGAGGAGGACTGGTTCAAACCGGCGGGAGCCTTCGACTCT | 560 |
| | | CATGTGCAGCCTCTGGGTTTACATTTAGTCGTTACCAAATGATGTGGGTACGCCAGGCCCC | |
| | | CGGAAAATGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTCTTGTTACCGTCTCAAGC | |
| | VL1 | GACATACAACTCACACAAAGTCCAAGCTCTCTTTCCCTTAGTCCAGGAGAACGCGTAACTC | 559 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTGGGTCAGGCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGCAAGGAAC | |
| | | TCGCCTGGAAATAAAA | |
| | VL2 | GACATACAGATGACTCAGAGCCCTGGTACACTGAGTGTATCTCCTGGGGAACGCGCCACTC | 561 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCTCCACTCACTTTTGGTTGTGG | |
| | | CACCAAAGTGGAGATAAAG | |
| EPB09 | VH1 | GAGGTGCAACTGCTTGAGTCAGGAGGAGGACTGGTTCAACCCGGCGGGAGCCTTCGACTCT | 552 |
| | | CATGTGCAGCCTCTGGGTTTACATTTAGTCGTTACCAAATGATGTGGGTACGCCÄGGCCCC | |
| | | CGGAAAAGGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTCTTGTTACCGTCTCAAGC | |
| | VH2 | GAGGTCCAGCTGGTTGAGTCTGGAGGTGGTGTAGTCAGGCCAGGAGGTTCCCTGCGTCTGT | 562 |
| | | CTTGCGCTGCCTCTGGGTTCACTGTGTCTGACTACAGTATGAACTGGGTTAGACAGGCTCC | |
| | | AGGGAAATGTTTGGAATGGATAGGGTTTATTCGCAATAAAGCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGGGTCAAGGAACTATGGTCACCGTCTCCTCT | |
| | VL1 | GACATACAGATGACTCAGAGCCCTGGTACACTGAGTGTATCTCCTGGGGAACGCGCCACTC | 554 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCTCCACTCACTTTTGGTGGTGG | |
| | | CACCAAAGTGGAGATAAAG | |
| | VL2 | GCCATACAGCTGACTCAATCACCTTCTTCTTTGAGCGCCTCTGTCGGTGATCGCGTTACAA | 563 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGATGTGGGAC | |
| | | TAAGCTGGAAATCAAA | |
| EPB10 | VH1 | CAAGTGCAACTCCTTGAAAGTGGAGGTGGCCTTGTCCAGCCAGGCGGTTCACTGCGTTTGA | 558 |
| | | GCTGTGCCGCATCCGGCTTTACCTTTTCCAGTTACACAATGAGTTGGGTCCGTCAAGCACC | |
| | | CGGGCAAGCTTTGGAATGGATGGGAACTATTTCATCTGGGGGACTTACACTTACTATCCC | |
| | | GATAGCGTCAAGGGACGTTTTACCATAAGCCGGGACAACGCTAAAAATTCACTCTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VH2 | GAGGTCCAGCTGGTTGAGTCTGGAGGTGGTGTAGTCAGGCCAGGAGGTTCCCTGCGTCTGT | 562 |
| | | CTTGCGCTGCCTCTGGGTTCACTGTGTCTGACTACAGTATGAACTGGGTTAGACAGGCTCC | |
| | | AGGGAAATGTTTGGAATGGATAGGGTTTATTCGCAATAAAGCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGCGTCAAGGAACTATGGTCACCGTCTCCTCT | |
| | VL1 | GACATACAACTCACACAAAGTCCAAGCTCTCTTTCCCTTAGTCCAGGAGAACGCGTAACTC | 550 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTGGGTCAGGCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGCAAGGAAC | |
| | | TCGCCTGGAAATAAAA | |
| | VL2 | GCCATACAGCTGACTCAATCACCTTCTTCTTTGAGCGCCTCTGTCGGTGATCGCGTTACAA | 563 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGATGTGGGAC | |
| | | TAAGCTGGAÀATCAAA | |
| EPB11 | VH1 | GAGGTGCAACTGCTTGAGTCAGGAGGAGGACTGGTTCAACCCGGCGGGAGCCTTCGACTCT | 552 |
| | | CATGTGCAGCCTCTGGGTTTACATTTAGTCGTTACCAAATGATGTGGGTACGCCAGGCCCC | |
| | | CGGAAAAGGTTTGGAGTGGGTGAGCAGCATTTCTCCTTCAGGAGGAGTGACACTTTACGCA | |
| | | GACAGTGTTAAGGGTCGGTTCACAATCAGTCGTGATAATTCCAAGAACACTCTCTATCTCC | |
| | | AAATGAACTCCCTTCGAGCTGAGGACACTGCTGTTTATTACTGTACTCGGGAACTTCTCGG | |
| | | CACTGTGGTTGTTCCTGTGGCTTGGAAGATGCGTGGATACTTCGACTATTGGGGGCAACTC | |
| | | ACTCTTGTTACCGTCTCAAGC | |
| | VH2 | CAAGTGCAACTCCTTGAAAGTGGAGGTGGCCTTGTCCAGCCAGGCGGTTCACTGCGTTTGA | 564 |
| | | GCTGTGCCGCATCCGGCTTTACCTTTTCCAGTTACACAATGAGTTGGGTCCGTCAAGCACC | |
| | | CGGGCAATGTTTGGAATGGATGGGAACTATTTCATCTGGTGGGACTTACACTTACTATCCC | |
| | | GATAGCGTCAAGGGACGTTTTACCATAAGCCGGGACAACGCTAAAAATTCACTCTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VL1 | GACATACAGATGACTCAGAGCCCTGGTACACTGAGTGTATCTCCTGGGGAACGCGCCACTC | 554 |
| | | TGTCTTGCCGAGCCTCTCAAAGCGTTAGTTCCAACCTGGCTTGGTATCAGCAAAAACCCGG | |
| | | TCAAGCACCTCGCTTGCTTATCTACGGGGCCTCCACACGGGCAACCGGCATTCCTGCACGC | |
| | | TTTAGTGGCTCTGGTAGCGGGACTGAATTCACTTTGACTATATCTTCCATGCAGAGTGAGG | |
| | | ATTTCGCCGTTTATTATTGTCAACAGTACAATAACTGGCCCCACTCACTTTTGGTGGTGG | |
| | | CACCAAAGTGGAGATAAAG | |
| | VL2 | GACATACAACTCACACAAAGTCCAAGCTCTCTTTCCCTTAGTCCAGGAGAACGCGTAACTC | 565 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTGGGTCAGGCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGTGTGGAAC | |
| | | TCGCCTGGAAATAAAA | |
| EPB12 | VH1 | GAGGTCCAGCTGGTTGAGTCTGGAGGTGGTGTAGTCAGGCCAGGAGGTTCCCTGCGTCTGT | 556 |
| | | CTTGCGCTGCCTCTGGGTTCACTGTGTCTGACTACAGTATGAACTGGGTTAGACAGGCTCC | |
| | | AGGGAAAGGTTTGGAATGGATAGGGTTTATTCGCAATAAAGCCAATGCCTATACCACCGAA | |
| | | TACAGCGCAAGCGTTAAAGGCAGGTTCACCATCAGTAGGGACGACTCTAAGAATACCCTCT | |
| | | ACCTGCAGATGAATAGCTTGAAGACAGAGGATACCGCTGTTTATTACTGCACTACCTACCC | |
| | | CAGGTATCATGCTATGGACAGTTGGGGTCAAGGAACTATGGTCACCGTCTCCTCT | |
| | VH2 | CAAGTGCAACTCCTTGAAAGTGGAGGTGGCCTTGTCCAGCCAGGCGGTTCACTGCGTTTGA | 564 |
| | | GCTGTGCCGCATCCGGCTTTACCTTTTCCAGTTACACAATGAGTTGGGTCCGTCAAGCACC | |
| | | CGGGCAATGTTTGGAATGGATGGGAACTATTTCATCTGGTGGGACTTACACTTACTATCCC | |
| | | GATAGCGTCAAGGGACGTTTTACCATAAGCCGGGACAACGCTAAAAATTCACTCTATCTTC | |
| | | AGATGAACAGTCTTAGGGCTGAGGATACCGCTGTTTATTATTGCGCCCGAGAAGCCATTTT | |
| | | CACCTACTGGGGGCGCGGTACACTTGTCACCGTTAGCAGT | |
| | VL1 | GCCATAGAGCTGACTCAATCACCTTCTTCTTTGAGCGCCTCTGTCGGTGATCGCGTTACAA | 557 |
| | | TTACATGCCGAGCTTCTCAGTCTATCTCTAATAACCTCCACTGGTATCTCCAGAAACCTGG | |
| | | ACAGTCACCACAGCTTCTGATATATTACGGCTTTCAGTCTATAAGTGGAGTCCCAAGTAGA | |
| | | TTTTCCGGTAGTGGATCTGGCACCGATTTTACTCTTACTATCTCTAGCCTCCAACCTGAAG | |
| | | ATTTCGCAACCTATTACTGCCAGCAGGCCAATTCCTGGCCCTTGACCTTCGGAGGTGGGAC | |
| | | TAAGCTGGAAATCAAA | |
| | VL2 | GACATACAACTCACACAAAGTCCAAGCTCTCTTTCCCTTAGTCCAGGAGAACGCGTAACTC | 565 |
| | | TCTCATGTAAAGCCTCCCAGGATATCAATAACTATCTCTCTTGGTACCAACAAAAGCCTGA | |
| | | CCAAGCTCCTAAGCTGCTTATCAAACGCGCCAATCGGTTGGTAGACGGAGTTCCCGACAGG | |
| | | TTCAGTGGGTCAGGCTCAGGTACAGATTTTACCTTGAAGATATCACGCGTGGAAGCCGAAG | |
| | | ACGTAGGGGTATATTATTGTCTGAAATATGACGAGTTCCCCTATACTTTTGGGTGTGGAAC | |
| | | TCGCCTGGAAATAAAA | |

Table 54 below shows the heavy chain and light chain CDR sequences of the engineered antibodies targeting EphA2.

TABLE 54

| Name | CDR | Sequence | SEQ ID NO |
|---|---|---|---|
| EPB01 | V1 CDR-H1 | RYQMM | 151 |
| | V1 CDR-H2 | SISPSGGVTLYADSVKG | 152 |
| | V1 CDR-H3 | ELLGTVVVPVAWKMRGYFDY | 153 |
| | V1 CDR-L1 | RASQSVSSNLA | 154 |
| | V1 CDR-L2 | GASTRAT | 155 |
| | V1 CDR-L3 | QQYNNWPPLT | 156 |
| | V2 CDR-H1 | HYMMA | 157 |
| | V2 CDR-H2 | RIGPSGGPTHYADSKG | 158 |
| | V2 CDR-H3 | YDSGYDYVAVAGPAEYFQH | 159 |
| | V2 CDR-L1 | RASQSISTWLA | 160 |
| | V2 CDR-L2 | KASNLHT | 161 |
| | V2 CDR-L3 | QQYNSYSRT | 162 |
| EPB02 | V1 CDR-H1 | DYSMN | 163 |
| | V1 CDR-H2 | FIRNKANAYTTEYSASVKG | 164 |
| | V1 CDR-H3 | YPRYHAMDSW | 165 |
| | V1 CDR-L1 | RASQSISNNLH | 166 |
| | V1 CDR-L2 | YGFQSIS | 167 |
| | V1 CDR-L3 | QQANSWPLT | 168 |
| | V2 CDR-H1 | HYMMA | 157 |
| | V2 CDR-H2 | RIGPSGGPTHYADSKG | 158 |
| | V2 CDR-H3 | YDSGYDYVAVAGPAEYFQH | 159 |
| | V2 CDR-L1 | RASQSISTWLA | 160 |
| | V2 CDR-L2 | KASNLHT | 161 |
| | V2 CDR-L3 | QQYNSYSRT | 162 |
| EPB03 | V1 CDR-H1 | SYTMS | 169 |
| | V1 CDR-H2 | TISSGGTYTYYPDSVKG | 170 |
| | V1 CDR-H3 | EAIFTYW | 171 |
| | V1 CDR-L1 | KASQDINNYLS | 172 |
| | V1 CDR-L2 | RANRLVD | 173 |
| | V1 CDR-L3 | LKYDEFPYT | 174 |
| | V2 CDR-H1 | HYMMA | 157 |
| | V2 CDR-H2 | RIGPSGGPTHYADSKG | 158 |
| | V2 CDR-H3 | YDSGYDYVAVAGPAEYFQH | 159 |
| | V2 CDR-L1 | RASQSISTWLA | 160 |
| | V2 CDR-L2 | KASNLHT | 161 |
| | V2 CDR-L3 | QQYNSYSRT | 162 |
| EPB04 | V1 CDR-H1 | HYMMA | 157 |
| | V1 CDR-H2 | RIGPSGGPTHYADSKG | 158 |
| | V1 CDR-H3 | YDSGYDYVAVAGPAEYFQH | 159 |
| | V1 CDR-L1 | RASQSISTWLA | 160 |
| | V1 CDR-L2 | KASNLHT | 161 |
| | V1 CDR-L3 | QQYNSYSRT | 162 |
| | V2 CDR-H1 | RYQMM | 151 |
| | V2 CDR-H2 | SISPSGGVTLYADSVKG | 152 |
| | V2 CDR-H3 | ELLGTYRGYFDY | 153 |
| | V2 CDR-L1 | RAS | 154 |
| | V2 CDR-L2 | GASTRAT | 155 |
| | V2 CDR-L3 | QQYNNWPPLT | 156 |
| EPB05 | V1 CDR-H1 | HYMMA | 157 |
| | V1 CDR-H2 | RIGPSGGPTHYADSKG | 158 |
| | V1 CDR-H3 | YDSGYDYVAVAGPAEYFQH | 159 |
| | V1 CDR-L1 | RASQSISTWLA | 160 |
| | V1 CDR-L2 | KASNLHT | 161 |
| | V1 CDR-L3 | QQYNSYSRT | 162 |
| | V2 CDR-H1 | DYSMN | 163 |
| | V2 CDR-H2 | FIRNKANAYTTEYSASVKG | 164 |
| | V2 CDR-H3 | YPRYHAMDSW | 165 |
| | V2 CDR-L1 | RASQSISNNLH | 166 |
| | V2 CDR-L2 | YGFQSIS | 167 |
| | V2 CDR-L3 | QQANSWPLT | 168 |
| EPB06 | V1 CDR-H1 | HYMMA | 157 |
| | V1 CDR-H2 | RIGPSGGPTHYADSKG | 158 |
| | V1 CDR-H3 | YDSGYDYVAVAGPAEYFQH | 159 |
| | V1 CDR-L1 | RASQSISTWLA | 160 |
| | V1 CDR-L2 | KASNLHT | 161 |
| | V1 CDR-L3 | QQYNSYSRT | 162 |
| | V2 CDR-H1 | SYTMS | 169 |
| | V2 CDR-H2 | TISSGGTYTYYPDSVKG | 170 |
| | V2 CDR-H3 | EAIFTYW | 171 |
| | V2 CDR-L1 | KASQDINNYLS | 172 |
| | V2 CDR-L2 | RANRLVD | 173 |
| | V2 CDR-L3 | LKYDEFPYT | 174 |
| EPB07 | V1 CDR-H1 | DYSMN | 163 |
| | V1 CDR-H2 | FIRNKANAYTTEYSASVKG | 164 |
| | V1 CDR-H3 | YPRYHAMDSW | 165 |
| | V1 CDR-L1 | RASQSISNNLH | 166 |
| | V1 CDR-L2 | YGFQSIS | 167 |
| | V1 CDR-L3 | QQANSWPLT | 168 |
| | V2 CDR-H1 | RYQMM | 151 |
| | V2 CDR-H2 | SISPSGGVTLYADSVKG | 152 |
| | V2 CDR-H3 | ELLGTYRGYFDY | 153 |
| | V2 CDR-L1 | RAS | 154 |
| | V2 CDR-L2 | GASTRAT | 155 |
| | V2 CDR-L3 | QQYNNWPPLT | 156 |
| EPB08 | V1 CDR-H1 | SYTMS | 169 |
| | V1 CDR-H2 | TISSGGTYTYYPDSVKG | 170 |
| | V1 CDR-H3 | EAIFTYW | 171 |
| | V1 CDR-L1 | KASQDINNYLS | 172 |
| | V1 CDR-L2 | RANRLVD | 173 |
| | V1 CDR-L3 | LKYDEFPYT | 174 |
| | V2 CDR-H1 | RYQMM | 151 |
| | V2 CDR-H2 | SISPSGGVTLYADSVKG | 152 |
| | V2 CDR-H3 | ELLGTYRGYFDY | 153 |
| | V2 CDR-L1 | RAS | 154 |
| | V2 CDR-L2 | GASTRAT | 155 |
| | V2 CDR-L3 | QQYNNWPPLT | 156 |
| EPB09 | V1 CDR-H1 | RYQMM | 151 |
| | V1 CDR-H2 | SISPSGGVTLYADSVKG | 152 |
| | V1 CDR-H3 | ELLGTYRGYFDY | 153 |
| | V1 CDR-L1 | RAS | 154 |
| | V1 CDR-L2 | GASTRAT | 155 |
| | V1 CDR-L3 | QQYNNWPPLT | 156 |
| | V2 CDR-H1 | DYSMN | 163 |
| | V2 CDR-H2 | FIRNKANAYTTEYSASVKG | 164 |
| | V2 CDR-H3 | YPRYHAMDSW | 165 |
| | V2 CDR-L1 | RASQSISNNLH | 166 |
| | V2 CDR-L2 | YGFQSIS | 167 |
| | V2 CDR-L3 | QQANSWPLT | 168 |
| EPB10 | V1 CDR-H1 | SYTMS | 169 |
| | V1 CDR-H2 | TISSGGTYTYYPDSVKG | 170 |
| | V1 CDR-H3 | EAIFTYW | 171 |
| | V1 CDR-L1 | KASQDINNYLS | 172 |
| | V1 CDR-L2 | RANRLVD | 173 |
| | V1 CDR-L3 | LKYDEFPYT | 174 |
| | V2 CDR-H1 | DYSMN | 163 |
| | V2 CDR-H2 | FIRNKANAYTTEYSASVKG | 164 |
| | V2 CDR-H3 | YPRYHAMDSW | 165 |
| | V2 CDR-L1 | RASQSISNNLH | 166 |
| | V2 CDR-L2 | YGFQSIS | 167 |
| | V2 CDR-L3 | QQANSWPLT | 168 |
| EPB11 | V1 CDR-H1 | RYQMM | 151 |
| | V1 CDR-H2 | SISPSGGVTLYADSVKG | 152 |
| | V1 CDR-H3 | ELLGTYRGYFDY | 153 |
| | V1 CDR-L1 | RAS | 154 |
| | V1 CDR-L2 | GASTRAT | 155 |
| | V1 CDR-L3 | QQYNNWPPLT | 156 |
| | V2 CDR-H1 | SYTMS | 169 |
| | V2 CDR-H2 | TISSGGTYTYYPDSVKG | 170 |
| | V2 CDR-H3 | EAIFTYW | 171 |
| | V2 CDR-L1 | KASQDINNYLS | 172 |
| | V2 CDR-L2 | RANRLVD | 173 |
| | V2 CDR-L3 | LKYDEFPYT | 174 |
| EPB12 | V1 CDR-H1 | DYSMN | 163 |
| | V1 CDR-H2 | FIRNKANAYTTEYSASVKG | 164 |
| | V1 CDR-H3 | YPRYHAMDSW | 165 |
| | V1 CDR-L1 | RASQSISNNLH | 166 |
| | V1 CDR-L2 | YGFQSIS | 167 |
| | V1 CDR-L3 | QQANSWPLT | 168 |
| | V2 CDR-H1 | SYTMS | 169 |
| | V2 CDR-H2 | TISSGGTYTYYPDSVKG | 170 |
| | V2 CDR-H3 | EAIFTYW | 171 |
| | V2 CDR-L1 | KASQDINNYLS | 172 |
| | V2 CDR-L2 | RANRLVD | 173 |
| | V2 CDR-L3 | LKYDEFPYT | 174 |

The EphA2 protein binding constants of EPB01, EPB02, EPB03, EPB04, EPB05, EPB06, EPB07, EPB08, EPB09, EPB10, EPB11, and EPB12 were determined using Octet Red96e (Sartorius). In order to analyze the binding constants of the twelve biparatopic antibodies, the human EphA2 recombinant protein (Sino Biologicals, 13926-H08H) was loaded onto the Anti-Penta-HIS (HIS1K) biosensor (Sartorius, 18-5120), and then the twelve antibodies were added in a binding reaction (300 seconds) and a dissociation reaction (1,200 seconds) at various concentrations. Based on the above, the affinity for EphA2 was calculated (Table 55). Table 55 below illustrates a result obtained by analyzing the binding constants of the engineered antibodies targeting EphA2.

TABLE 55

| Antibodies | K_{D} (nM) | Kₐ (1/Ms) | K_{d} (1/s) |
|---|---|---|---|
| EPB01 | 9.8579 | 2.32E+05 | 2.29E−03 |
| EPB02 | <0.001 | 2.15E+05 | <1.0E−07 |
| EPB03 | 0.7944 | 4.91E+05 | 3.90E−04 |
| EPB04 | 2.8644 | 4.95E+05 | 1.42E−03 |
| EPB05 | <0.001 | 3.42E+05 | <1.0E−07 |
| EPB06 | <0.001 | 3.84E+05 | <1.0E−07 |
| EPB07 | 0.0795 | 2.03E+05 | 1.62E−05 |
| EPB08 | 3.2174 | 6.58E+05 | 2.12E−03 |
| EPB09 | 2.3522 | 2.64E+05 | 6.20E−04 |
| EPB10 | 0.1878 | 3.92E+05 | 7.36E−05 |
| EPB11 | 0.5349 | 2.98E+05 | 1.59E−04 |
| EPB12 | 0.8657 | 5.44E+05 | 4.71E−04 |

PC-3 prostate cancer cell line was used to analyze the ability of antibodies to inhibit EphA2 signaling. PC-3 cancer cell lysate treated with each antibody at a concentration of 50 nM for 30 minutes was analyzed by western blot. The 1C1 humanized antibody that targets human EphA2 was used as a positive control and was prepared based on the sequence published in the literature (Kinch et al., US 20090304721 A1). Akt Rabbit mAb (Cell Signaling Technology, 9272), Phospho-Akt (Ser473) (D9E) XP® Rabbit mAb (Cell Signaling Technology, 4060), and β-Actin (13E5) Rabbit mAb (Cell Signaling Technology, 4970) were used as primary antibodies for analysis, and anti-rabbit IgG, HRP-linked antibody (Cell Signaling Technology, 7074) was used as secondary antibody. In the analysis of AKT signaling pathway inhibition, EPB02, EPB03, and EPB05 showed a similar level of inhibition as the positive control 1C1 (FIG. 44).

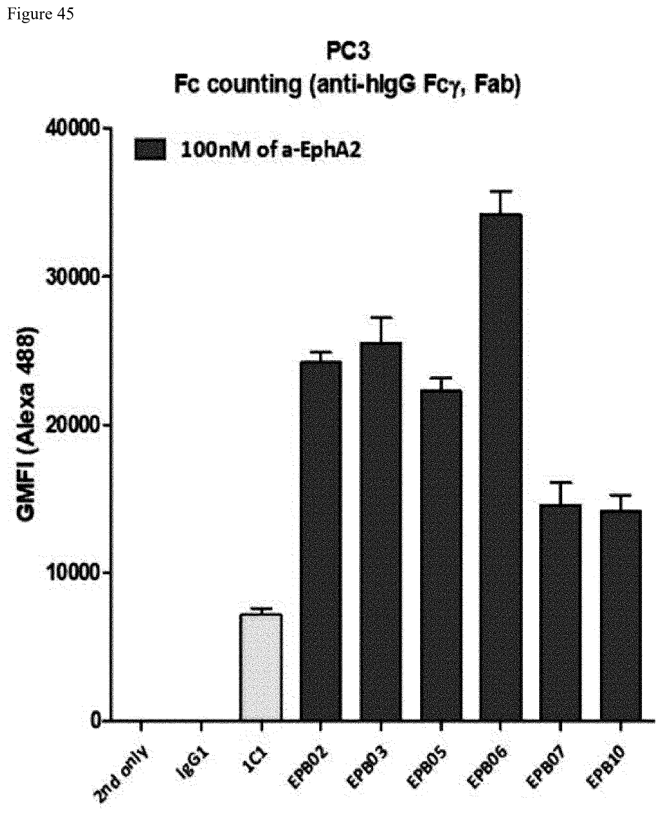

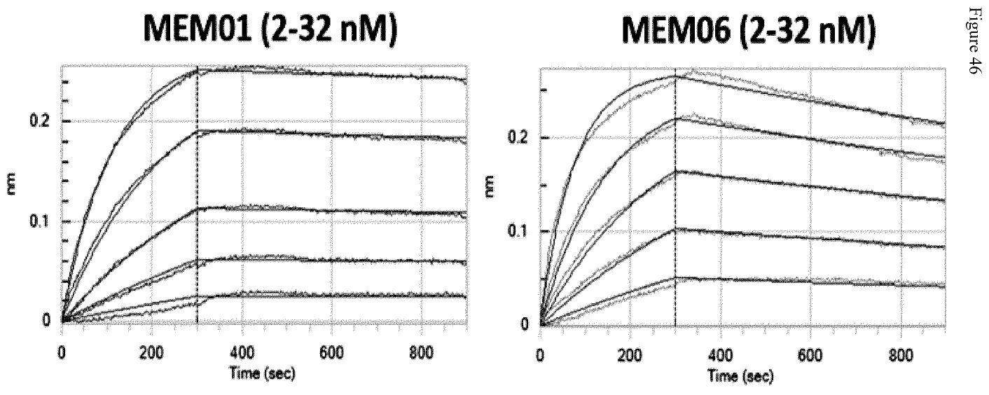

PC-3 prostate cancer cell line was used to quantify the Fc loads on the surface of EphA2 expressing cells. 100 nM antibodies were allowed to bind to the PC-3 cell line at 4° C. for 30 minutes, and the Fc loads were quantified using the Alexa 488 fluorescence-conjugated anti-human IgG Fcγ Fab antibody (Jackson ImmunoResearch, 109-547-008) (FIG. 44). It was shown that higher Fc loads on the surface of cancer cells are induced by treatment of EPB02, EPB03, EPB05, EPB06, EPB07, and EPB10 compared to treatment of 1C1 humanized antibody that targets EphA2 (FIG. 45).

### Example 19. Design, Preparation and Analysis of Antibody Structure Targeting MET

The variant light chain and heavy chain polypeptide sequences of the antibodies that specifically bind to the MET protein are shown in Table 56. For MEM01, expression vectors consisting of the sequences corresponding to Fc-Hole (SEQ ID NO: 7), MEM01 HC (SEQ ID NO: 568), and MEM01 LC (SEQ ID NO: 569) were co-transfected into EXPICHO-S™ (Gibco, A29127) (FIG. 41a, Table 56), and purification and analysis were performed in the same manner as described in Example 1. Expression, purification and analysis were performed for MEM06 in the same manner as mentioned above (Table 56, Table 57).

TABLE 56

| Name | Sequence | SEQ ID NO |
|---|---|---|
| MEM01 HC | EVQLVESGGGLVQPGGSLRLSCAASGYTFT | 568 |
| | SYWLHWVRQAPGKCLEWVGMIDPSNSDTRF | |
| | NPNFKDRFTISADTSKNTAYLQMNSLRAED | |
| | TAVYYCATYRSYVTPLDYWGQGTLVTVSSA | |
| | STKGPSVFPLAPSSKSTSGGTAALGCLVKD | |
| | YFPEPVTVSWNSGALTSGVHTFPAVLQSSG | |
| | LYSLSSVVTVPSSSLGTQTYICNVNHKPSN | |
| | TKVDKKVEPKSCDKTHTCPPCPAPELLGGP | |
| | SVFLFPPKPKDTLMISRTPEVTCVVVDVSH | |
| | EDPEVKFNWYVDGVEVHNAKTKPREEQYNS | |
| | TYRVVSVLTVLHQDWLNGKEYKCKVSNKAL | |
| | PAPIEKTISKAKGQPREPQVYTLPPCREEM | |
| | TKNQVSLWCLVKGFYPSDIAVEWESNGQPE | |
| | NNYKTTPPVLDSDGSFFLYSKLTVDKSRWQ | |
| | QGNVFSCSVMHEALHNHYTQKSLSLSPGK | |
| MEM01 LC | DIQMTQSPSSLSASVGDRVTITCKSSQSLL | 569 |
| | YTSSQKNYLAWYQQKPGKAPKLLIYWASTR | |
| | ESGVPSRFSGSGSGTDFTLTISSLQPEDFA | |
| | TYYCQQYYAYPWTFGCGTKVEIKRTVAAPS | |
| | VFIFPPSDEQLKSGTASVVCLLNNFYPREA | |
| | KVQWKVDNALQSGNSQESVTEQDSKDSTYS | |
| | LSSTLTLSKADYEKHKVYACEVTHQGLSSP | |
| | VTKSFNRGECGGGGSGGGGSGGGGSEPKSS | |
| | DKTHTCPPCPAPELLGGPSVFLFPPKPKDT | |
| | LMISRTPEVTCVVVDVSHEDPEVKFNWYVD | |
| | GVEVHNAKTKPREEQYNSTYRVVSVLTVLH | |
| | QDWLNGKEYKCKVSNKALPAPIEKTISKAK | |
| | GQPREPQVYTLPPCREEMTKNQVSLWCLVK | |
| | GFYPSDIAVEWESNGQPENNYKTTPPVLDS | |
| | DGSFFLYSKLTVDKSRWQQGNVFSCSVMHE | |
| | ALHNHYTQKSLSLSPGK | |
| MEM06 HC | QVQLVQSGAEVKKPGASVKVSCKASGYTFT | 570 |
| | SYGFSWVRQAPGQCLEWMGWISASNGNTYY | |
| | AQKLQGRVTMTTDTSTSTAYMELRSLRSDD | |
| | TAVYYCARVYADYADYWGQGTLVTVSSAST | |
| | KGPSVFPLAPSSKSTSGGTAALGCLVKDYF | |
| | PEPVTVSWNSGALTSGVHTFPAVLQSSGLY | |
| | SLSSVVTVPSSSLGTQTYICNVNHKPSNTK | |
| | VDKKVEPKSCDKTHTCPPCPAPELLGGPSV | |
| | FLFPPKPKDTLMISRTPEVTCVVVDVSHED | |
| | PEVKFNWYVDGVEVHNAKTKPREEQYNSTY | |
| | RVVSVLTVLHQDWLNGKEYKCKVSNKALPA | |
| | PIEKTISKAKGQPREPQVYTLPPCREEMTK | |
| | NQVSLWCLVKGFYPSDIAVEWESNGQPENN | |
| | YKTTPPVLDSDGSFFLYSKLTVDKSRWQQG | |
| | NVFSCSVMHEALHNHYTQKSLSLSPGK | |
| MEM06 LC | DIQMTQSPSSVSASVGDRVTITCRASQGIN | 571 |
| | TWLAWYQQKPGKAPKLLIYAASSLKSGVPS | |
| | RFSGSGSGTDFTLTISSLQPEDFATYYCQQ | |
| | ANSFPLTFGCGTKVEIKRTVAAPSVFIFPP | |
| | SDEQLKSGTASVVCLLNNFYPREAKVQWKV | |
| | DNALQSGNSQESVTEQDSKDSTYSLSSTLT | |
| | LSKADYEKHKVYACEVTHQGLSSPVTKSFN | |
| | RGECGGGGSGGGGSGGGGSEPKSSDKTHTC | |
| | PPCPAPELLGGPSVFLFPPKPKDTLMISRT | |
| | PEVTCVVVDVSHEDPEVKFNWYVDGVEVHN | |
| | AKTKPREEQYNSTYRVVSVLTVLHQDWLNG | |
| | KEYKCKVSNKALPAPIEKTISKAKGQPREP | |
| | QVYTLPPCREEMTKNQVSLWCLVKGFYPSD | |
| | IAVEWESNGQPENNYKTTPPVLDSDGSFFL | |
| | YSKLTVDKSRWQQGNVFSCSVMHEALHNHY | |
| | TQKSLSLSPGK | |

TABLE 57

| | | SEQ ID |
|---|---|---|
| Name | Sequence | NO |
| MEM01 HC | GAAGTCCAATTGGTCGAGAGTGGAGGCGGG | 572 |
| | TTGGTACAGCCAGGAGGTTCACTTAGGTTG | |
| | TCCTGCGCCGCCAGTGGTTACACTTTCACA | |
| | TCTTATTGGCTGCACTGGGTGCGCCAAGCT | |
| | CCTGGGAAGTGTCTCGAATGGGTGGGTATG | |
| | ATCGATCCATCTAATTCCGACACACGGTTT | |
| | AACCCAAATTTTAAGGATAGATTTACAATT | |
| | AGTGCTGACACTTCAAAAAACACAGCATAC | |
| | CTCCAGATGAACAGCCTGCGTGCTGAGGAT | |
| | ACTGCTGTCTACTACTGTGCAACTTACCGC | |
| | TCCTATGTCACACCTTTGGATTATTGGGGC | |
| | CAGGGGACTCTGGTGACCGTGAGTTCTGCT | |
| | AGCACCAAAGGACCTAGTGTTTTTCCTCTT | |
| | GCCCCTTCCTCAAAGTCTACCTCTGGGGGG | |
| | ACAGCCGCTCTGGGCTGCCTGGTCAAGGAT | |
| | TATTTCCCAGAGCCTGTCACTGTCAGTTGG | |
| | AACTCTGGAGCCTTGACTTCTGGTGTTCAT | |
| | ACATTTCCTGCTGTCCTTCAAAGCAGCGGC | |
| | TTGTACTCATTGTCTTCTGTTGTGACAGTA | |
| | CCCTCAAGCAGCCTCGGCACTCAGACATAC | |
| | ATCTGCAATGTCAACCACAAACCCTCAAAT | |
| | ACAAAGGTAGATAAAAAAGTCGAACCAAAG | |
| | TCTTGTGACAAAACTCACACGTGCCCACCG | |
| | TGCCCAGCACCTGAACTCCTGGGGGGACCG | |
| | TCAGTCTTCCTCTTCCCCCCAAAACCCAAG | |
| | GACACCCTCATGATCTCCCGGACCCCTGAG | |
| | GTCACATGCGTGGTGGTGGACGTGAGCCAC | |
| | GAAGACCCTGAGGTCAAGTTCAACTGGTAC | |
| | GTGGACGGCGTGGAGGTGCATAATGCCAAG | |
| | ACAAAGCCGCGGGAGGAGCAGTACAACAGC | |
| | ACGTACCGTGTGGTCAGCGTCCTCACCGTC | |
| | CTGCACCAGGACTGGCTGAATGGCAAGGAG | |
| | TACAAGTGCAAGGTCTCCAACAAAGCCCTC | |
| | CCAGCCCCCATCGAGAAAACCATCTCCAAA | |
| | GCCAAAGGGCAGCCCCGAGAACCACAGGTG | |
| | TACACCCTGCCCCCATGCCGGGAGGAGATG | |
| | ACCAAGAACCAGGTCAGCCTGTGGTGCCTG | |
| | GTCAAAGGCTTCTATCCCAGCGACATCGCC | |
| | GTGGAGTGGGAGAGCAATGGGCAGCCGGAG | |
| | AACAACTACAAGACCACGCCTCCCGTGCTG | |
| | GACTCCGACGGCTCCTTCTTCCTCTACAGC | |
| | AAGCTCACCGTGGACAAGAGCAGGTGGCAG | |
| | CAGGGGAACGTCTTCTCATGCTCCGTGATG | |
| | CATGAGGCTCTGCACAACCACTACACGCAG | |
| | AAGAGCCTCTCCCTGTCTCCGGGTAAA | |
| MEM01 LC | GATATACAGATGACACAAAGTCCCTCATCA | 573 |
| | CTTTCTGCCTCCGTTGGAGATCGTGTGACC | |
| | ATTACCTGTAAGAGTTCCCAATCACTGCTT | |
| | TATACCTCTTCACAAAAAAATTACCTCGCT | |
| | TGGTACCAGCAGAAGCCAGGTAAAGCACCT | |
| | AAGCTGTTGATCTATTGGGCCTCCACTAGA | |
| | GAGTCAGGCGTGCCCAGCCGTTTCTCCGGT | |
| | TCAGGGAGTGGGACAGACTTTACCTTGACC | |
| | ATTTCTTCTTTGCAACCTGAAGACTTCGCC | |
| | ACATACTATTGTCAGCAATATTACGCATAT | |
| | CCATGGACCTTTGGGTGTGGAACCAAAGTC | |
| | GAAATAAAACGTACGGTGGCAGCTCCCAGC | |
| | GTTTTTATCTTTCCCCCATCCGACGAGCAG | |
| | CTCAAGAGTGGCACTGCCTCTGTAGTTTGT | |
| | TTGCTGAATAACTTCTATCCACGTGAAGCA | |
| | AAAGTACAGTGGAAGGTCGATAATGCCCTT | |
| | CAGAGCGGTAACAGCCAAGAAAGTGTTACC | |
| | GAGCAAGATTCCAAAGATTCCACTTACAGT | |
| | CTGTCCAGCACATTGACACTGAGTAAGGCT | |
| | GATTACGAAAAACACAAGGTGTACGCATGC | |
| | GAGGTGACACACCAAGGTCTTTCATCTCCT | |
| | GTAACTAAGAGCTTTAACCGGGGAGAATGT | |
| | GGTGGTGGGGGCAGCGGGGGCGGAGGTAGT | |
| | GGAGGCGGCGGTAGTGAACCAAAGAGTAGT | |
| | GACAAAACTCACACGTGCCCACCGTGCCCA | |
| | GCACCTGAACTCCTGGGGGGACCGTCAGTC | |
| | TTCCTCTTCCCCCCAAAACCCAAGGACACC | |
| | CTCATGATCTCCCGGACCCCTGAGGTCACA | |
| | TGCGTGGTGGTGGACGTGAGCCACGAAGAC | |
| | CCTGAGGTCAAGTTCAACTGGTACGTGGAC | |
| | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCCCAGCC | |
| | CCCATCGAGAAAACCATCTCCAAAGCCAAA | |
| | GGGCAGCCCCGAGAACCACAGGTGTACACC | |
| | CTGCCCCCATGCCGGGAGGAGATGACCAAG | |
| | AACCAGGTCAGCCTGTGGTGCCTGGTCAAA | |
| | GGCTTCTATCCCAGCGACATCGCCGTGGAG | |
| | TGGGAGAGCAATGGGCAGCCGGAGAACAAC | |
| | TACAAGACCACGCCTCCCGTGCTGGACTCC | |
| | GACGGCTCCTTCTTCCTCTACAGCAAGCTC | |
| | ACCGTGGACAAGAGCAGGTGGCAGCAGGGG | |
| | AACGTCTTCTCATGCTCCGTGATGCATGAG | |
| | GCTCTGCACAACCACTACACGCAGAAGAGC | |
| | CTCTCCCTGTCTCCGGGTAAA | |
| MEM06 HC | CAAGTGCAGCTTGTTCAATCAGGGGCCGAG | 574 |
| | GTTAAGAAACCAGGTGCTTCCGTCAAGGTC | |
| | TCCTGCAAGGCTTCCGGCTACACTTTTACC | |
| | AGTTATGGGTTCAGTTGGGTTAGACAGGCC | |
| | CCAGGGCAGTGTCTCGAATGGATGGGATGG | |
| | ATTTCCGCATCTAACGGGAATACTTACTAT | |
| | GCCCAGAAACTTCAAGGTAGGGTTACCATG | |
| | ACTACCGATACTTCCACTAGTACAGCCTAC | |
| | ATGGAACTCAGATCACTCCGTTCAGATGAC | |
| | ACCGCAGTATATTACTGTGCAAGGGTATAT | |
| | GCTGATTATGCCGATTATTGGGGGCAAGGA | |
| | ACACTTGTCACAGTATCCAGCGCTAGCACC | |
| | AAAGGACCTAGTGTTTTTCCTCTTGCCCCT | |
| | TCCTCAAAGTCTACCTCTGGGGGGACAGCC | |
| | GCTCTGGGCTGCCTGGTCAAGGATTATTTC | |
| | CCAGAGCCTGTCACTGTCAGTTGGAACTCT | |
| | GGAGCCTTGACTTCTGGTGTTCATACATTT | |
| | CCTGCTGTCCTTCAAAGCAGCGGCTTGTAC | |
| | TCATTGTCTTCTGTTGTGACAGTACCCTCA | |
| | AGCAGCCTCGGCACTCAGACATACATCTGC | |
| | AATGTCAACCACAAACCCTCAAATACAAAG | |
| | GTAGATAAAAAAGTCGAACCAAAGTCTTGT | |
| | GACAAAACTCACACGTGCCCACCGTGCCCA | |
| | GCACCTGAACTCCTGGGGGGACCGTCAGTC | |
| | TTCCTCTTCCCCCCAAAACCCAAGGACACC | |
| | CTCATGATCTCCCGGACCCCTGAGGTCACA | |
| | TGCGTGGTGGTGGACGTGAGCCACGAAGAC | |
| | CCTGAGGTCAAGTTCAACTGGTACGTGGAC | |
| | GGCGTGGAGGTGCATAATGCCAAGACAAAG | |
| | CCGCGGGAGGAGCAGTACAACAGCACGTAC | |
| | CGTGTGGTCAGCGTCCTCACCGTCCTGCAC | |
| | CAGGACTGGCTGAATGGCAAGGAGTACAAG | |
| | TGCAAGGTCTCCAACAAAGCCCTCCCAGCC | |
| | CCCATCGAGAAAACCATCTCCAAAGCCAAA | |
| | GGGCAGCCCCGAGAACCACAGGTGTACACC | |
| | CTGCCCCCATGCCGGGAGGAGATGACCAAG | |
| | AACCAGGTCAGCCTGTGGTGCCTGGTCAAA | |
| | GGCTTCTATCCCAGCGACATCGCCGTGGAG | |
| | TGGGAGAGCAATGGGCAGCCGGAGAACAAC | |
| | TACAAGACCACGCCTCCCGTGCTGGACTCC | |
| | GACGGCTCCTTCTTCCTCTACAGCAAGCTC | |
| | ACCGTGGACAAGAGCAGGTGGCAGCAGGGG | |
| | AACGTCTTCTCATGCTCCGTGATGCATGAG | |
| | GCTCTGCACAACCACTACACGCAGAAGAGC | |
| | CTCTCCCTGTCTCCGGGTAAA | |
| MEM06 LC | GATATCCAAATGACACAGTCACCCTCAAGT | 575 |
| | GTTAGCGCAAGTGTCGGGGACAGGGTGACC | |
| | ATCACATGCAGAGCTTCCCAGGGTATCAAT | |
| | ACATGGCTGGCATGGTATCAACAGAAACCC | |
| | GGAAAAGCACCAAAATTGCTTATTTATGCA | |
| | GCTTCTAGCTTGAAGAGTGGGGTTCCCTCT | |
| | CGTTTCTCTGGTTCAGGAAGCGGTACTGAC | |
| | TTTACCTTGACCATCAGTAGCTTGCAGCCC | |
| | GAAGATTTCGCTACATATTATTGCCAACAG | |
| | GCCAACTCTTTTCCCCTGACATTCGGTTGT | |
| | GGCACTAAAGTGGAAATTAAGCGTACGGTG | |
| | GCAGCTCCCAGCGTTTTTATCTTTCCCCCA | |
| | TCCGACGAGCAGCTCAAGAGTGGCACTGCC | |
| | TCTGTAGTTTGTTTGCTGAATAACTTCTAT | |
| | CCACGTGAAGCAAAAGTACAGTGGAAGGTC | |
| | GATAATGCCCTTCAGAGCGGTAACAGCCAA | |
| | GAAAGTGTTACCGAGCAAGATTCCAAAGAT | |
| | TCCACTTACAGTCTGTCCAGCACATTGACA | |
| | CTGAGTAAGGCTGATTACGAAAAACACAAG | |
| | GTGTACGCATGCGAGGTGACACACCAAGGT | |
| | CTTTCATCTCCTGTAACTAAGAGCTTTAAC | |
| | CGGGGAGAATGTGGTGGTGGGGGCAGCGGG | |
| | GGCGGAGGTAGTGGAGGCGGCGGTAGTGAA | |
| | CCAAAGAGTAGTGACAAAACTCACACGTGC | |
| | CCACCGTGCCCAGCACCTGAACTCCTGGGG | |
| | GGACCGTCAGTCTTCCTCTTCCCCCCAAAA | |
| | CCCAAGGACACCCTCATGATCTCCCGGACC | |
| | CCTGAGGTCACATGCGTGGTGGTGGACGTG | |
| | AGCCACGAAGACCCTGAGGTCAAGTTCAAC | |
| | TGGTACGTGGACGGCGTGGAGGTGCATAAT | |
| | GCCAAGACAAAGCCGCGGGAGGAGCAGTAC | |
| | AACAGCACGTACCGTGTGGTCAGCGTCCTC | |
| | ACCGTCCTGCACCAGGACTGGCTGAATGGC | |
| | AAGGAGTACAAGTGCAAGGTCTCCAACAAA | |
| | GCCCTCCCAGCCCCCATCGAGAAAACCATC | |
| | TCCAAAGCCAAAGGGCAGCCCCGAGAACCA | |
| | CAGGTGTACACCCTGCCCCCATGCCGGGAG | |
| | GAGATGACCAAGAACCAGGTCAGCCTGTGG | |
| | TGCCTGGTCAAAGGCTTCTATCCCAGCGAC | |
| | ATCGCCGTGGAGTGGGAGAGCAATGGGCAG | |
| | CCGGAGAACAACTACAAGACCACGCCTCCC | |
| | GTGCTGGACTCCGACGGCTCCTTCTTCCTC | |
| | TACAGCAAGCTCACCGTGGACAAGAGCAGG | |
| | TGGCAGCAGGGGAACGTCTTCTCATGCTCC | |
| | GTGATGCATGAGGCTCTGCACAACCACTAC | |
| | ACGCAGAAGAGCCTCTCCCTGTCTCCGGGT | |
| | AAA | |

Table 58 below shows the polypeptide sequences of the heavy chain variable region (VH) and light chain variable region (VL) of the engineered antibodies targeting MET. Table 59 below shows the nucleotide sequences of the heavy chain variable region (VH) and light chain variable region (VL) of the engineered antibodies targeting MET.

TABLE 58

| Name | | Sequence | SEQ ID NO |
|---|---|---|---|
| MEM01 | VH | EVQLVESGGGLVQPGGSLRLSCAASGYTFT | 576 |
| | | SYWLHWVRQAPGKCLEWVGMIDPSNSDTRF | |
| | | NPNFKDRFTISADTSKNTAYLQMNSLRAED | |
| | | TAVYYCATYRSYVTPLDYWGQGTLVTVSS | |
| | VL | DIQMTQSPSSLSASVGDRVTITCKSSQSLL | 577 |
| | | YTSSQKNYLAWYQQKPGKAPKLLIYWASTR | |
| | | ESGVPSRFSGSGSGTDFTLTISSLQPEDFA | |
| | | TYYCQQYYAYPWTFGCGTKVEIK | |
| MEM06 | VH | QVQLVQSGAEVKKPGASVKVSCKASGYTFT | 578 |
| | | SYGFSWVRQAPGQCLEWMGWISASNGNTYY | |
| | | AQKLQGRVTMTTDTSTSTAYMELRSLRSDD | |
| | | TAVYYCARVYADYADYWGQGTLVTVSS | |
| | VL | DIQMTQSPSSVSASVGDRVTITCRASQGIN | 579 |
| | | TWLAWYQQKPGKAPKLLIYAASSLKSGVPS | |
| | | RFSGSGSGTDFTLTISSLQPEDFATYYCQQ | |
| | | ANSFPLTFGCGTKVEIK | |

TABLE 59

| Name | | Sequence | SEQ ID NO |
|---|---|---|---|
| MEM01 | VH | GAAGTCCAATTGGTCGAGAGTGGAGGCGGG | 580 |
| | | TTGGTACAGCCAGGAGGTTCACTTAGGTTG | |
| | | TCCTGCGCCGCCAGTGGTTACACTTTCACA | |
| | | TCTTATTGGCTGCACTGGGTGCGCCAAGCT | |
| | | CCTGGGAAGTGTCTCGAATGGGTGGGTATG | |
| | | ATCGATCCATCTAATTCCGACACACGGTTT | |
| | | AACCCAAATTTTAAGGATAGATTTACAATT | |
| | | AGTGCTGACACTTCAAAAAACACAGCATAC | |
| | | CTCCAGATGAACAGCCTGCGTGCTGAGGAT | |
| | | ACTGCTGTCTACTACTGTGCAACTTACCGC | |
| | | TCCTATGTCACACCTTTGGATTATTGGGGC | |
| | | CAGGGGACTCTGGTGACCGTGAGTTCT | |
| | VL | GATATACAGATGACACAAAGTCCCTCATCA | 581 |
| | | CTTTCTGCCTCCGTTGGAGATCGTGTGACC | |
| | | ATTACCTGTAAGAGTTCCCAATCACTGCTT | |
| | | TATACCTCTTCACAAAAAAATTACCTCGCT | |
| | | TGGTACCAGCAGAAGCCAGGTAAAGCACCT | |
| | | AAGCTGTTGATCTATTGGGCCTCCACTAGA | |
| | | GAGTCAGGCGTGCCCAGCCGTTTCTCCGGT | |
| | | TCAGGGAGTGGGACAGACTTTACCTTGACC | |
| | | ATTTCTTCTTTGCAACCTGAAGACTTCGCC | |
| | | ACATACTATTGTCAGCAATATTACGCATAT | |
| | | CCATGGACCTTTGGGTGTGGAACCAAAGTC | |
| | | GAAATAAAA | |
| MEM06 | VH | CAAGTGCAGCTTGTTCAATCAGGGGCCGAG | 582 |
| | | GTTAAGAAACCAGGTGCTTCCGTCAAGGTC | |
| | | TCCTGCAAGGCTTCCGGCTACACTTTTACC | |
| | | AGTTATGGGTTCAGTTGGGTTAGACAGGCC | |
| | | CCAGGGCAGTGTCTCGAATGGATGGGATGG | |
| | | ATTTCCGCATCTAACGGGAATACTTACTAT | |
| | | GCCCAGAAACTTCAAGGTAGGGTTACCATG | |
| | | ACTACCGATACTTCCACTAGTACAGCCTAC | |
| | | ATGGAACTCAGATCACTCCGTTCAGATGAC | |
| | | ACCGCAGTATATTACTGTGCAAGGGTATAT | |
| | | GCTGATTATGCCGATTATTGGGGGCAAGGA | |
| | | ACACTTGTCACAGTATCCAGC | |
| | VL | GATATCCAAATGACACAGTCACCCTCAAGT | 583 |
| | | GTTAGCGCAAGTGTCGGGGACAGGGTGACC | |
| | | ATCACATGCAGAGCTTCCCAGGGTATCAAT | |
| | | ACATGGCTGGCATGGTATCAACAGAAACCC | |
| | | GGAAAAGCACCAAAATTGCTTATTTATGCA | |
| | | GCTTCTAGCTTGAAGAGTGGGGTTCCCTCT | |
| | | CGTTTCTCTGGTTCAGGAAGCGGTACTGAC | |
| | | TTTACCTTGACCATCAGTAGCTTGCAGCCC | |
| | | GAAGATTTCGCTACATATTATTGCCAACAG | |
| | | GCCAACTCTTTTCCCCTGACATTCGGTTGT | |
| | | GGCACTAAAGTGGAAATTAAG | |

Table 60 below shows the heavy chain and light chain CDR sequences of the engineered antibodies targeting MET.

TABLE 60

| Name | CDR | Sequence | SEQ ID NO |
|---|---|---|---|
| MEM01 | CDR-H1 | SYWLH | 236 |
| | CDR-H2 | MIDPSNSDTRFNPNFKD | 237 |
| | CDR-H3 | YRSYVTPLDY | 238 |
| | CDR-L1 | KSSQSLLYTSSQKNYLA | 239 |
| | CDR-L2 | WASTRES | 240 |
| | CDR-L3 | QQYYAYPWT | 241 |
| MEM06 | CDR-H1 | SYGFS | 584 |
| | CDR-H2 | WISASNGNTYYAQKLQG | 585 |
| | CDR-H3 | VYADYADY | 586 |
| | CDR-L1 | QQANSFPLT | 587 |
| | CDR-L2 | AASSLKS | 588 |
| | CDR-L3 | QQANSFPLT | 589 |

The MET protein binding constants of MEM01 and MEM06 were determined using Octet Red96e (Sartorius). In order to analyze the binding constants of the antibodies, the antibodies were loaded onto the anti-human Fab-CH1 2nd generation (FAB2G) biosensor (Sartorius, 18-5125). Then, the human MET recombinant protein (Sino Biologicals, 10692-H08H) were added in a binding reaction (300 seconds) and a dissociation reaction (600 seconds) at various concentrations (FIG. 46), and the affinities of antibodies for MET was calculated (FIG. 46, Table 61). Table 61 below illustrates the binding constants of the engineered antibodies targeting MET.

TABLE 61

| Antibodies | K_{D} (nM) | Kₐ (1/Ms) | K_{d} (1/s) |
|---|---|---|---|
| MEM01 | 0.1861 | 3.31E+05 | 6.16E−05 |
| MEM06 | 0.7881 | 4.20E+05 | 3.31E−04 |

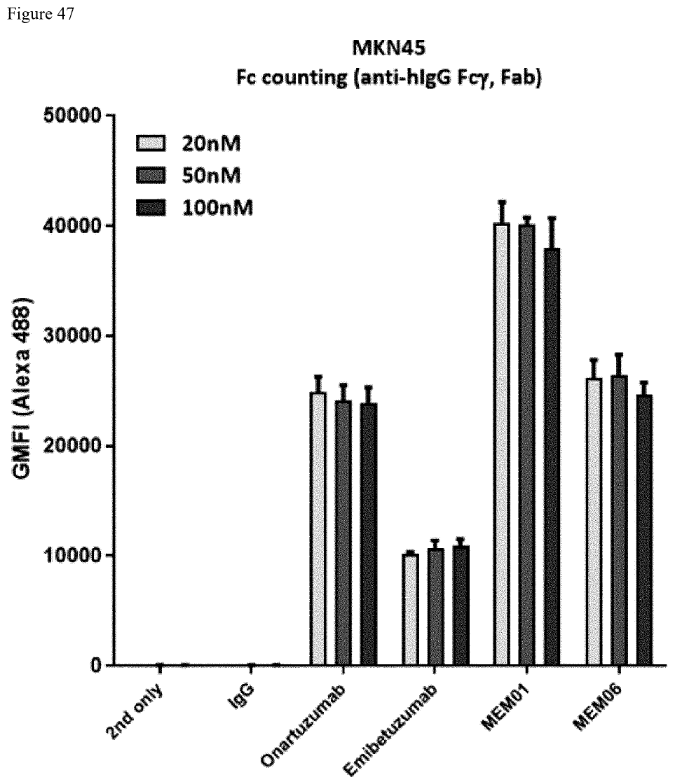

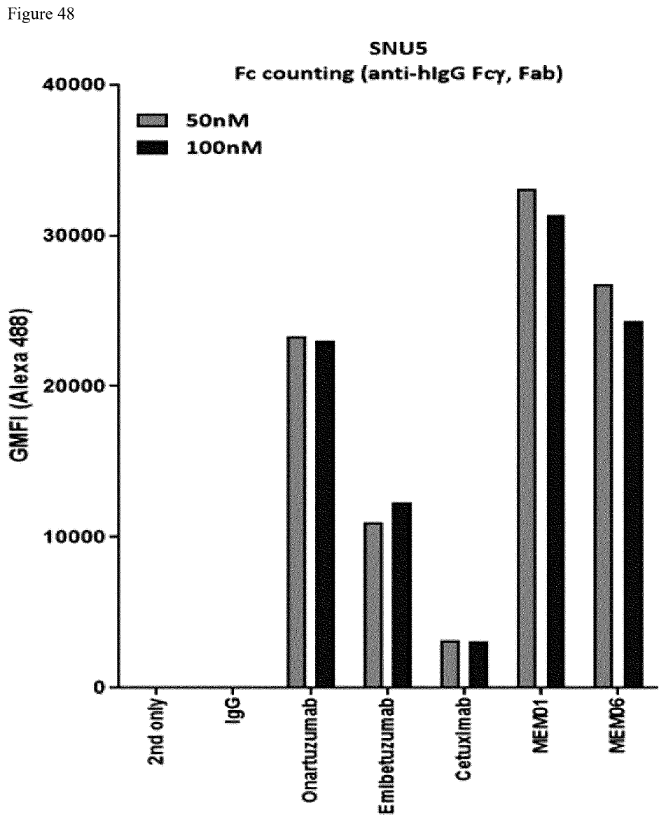

MKN45 and SNU-5 gastric cancer cell lines were used to quantify the Fc loads on the surface of MET expressing cells. The human IgG1 control, onartuzumab (produced in CHO cell line), emibetuzumab, MEM01I, and MEM06 antibodies were allowed to bind to the cell lines at 4° C. for 30 minutes, and the Fc loads were quantified using the Alexa 488 fluorescence-conjugated anti-human IgG Fcγ Fab antibody (Jackson ImmunoResearch, 109-547-008) (FIGS. 47 and 48). It was shown that high Fc loads on the surface of MET expressing cancer cells are induced by treatment of MEM01 compared to onartuzumab and emibetuzumab, which target MET (FIGS. 47 and 48). Treatment of MEM06 induced higher Fc loads on the surface of MET expressing cancer cells compared to emibetuzumab, but a similar level when compared to onartuzumab (FIGS. 47 and 48).

### Example 20. Design, Preparation and Analysis of Antibody Structure Targeting EGFR

The variant light chain and heavy chain polypeptide sequences of the antibodies that specifically bind to the EGFR protein are shown in Table 62. For EGM01, expression vectors consisting of the sequences corresponding to Fc-Hole (SEQ ID NO: 7), EGM01 HC (SEQ ID NO: 590), and EGF01 LC (SEQ ID NO: 591) were co-transfected into EXPICHOWS™ (Gibco, A29127) (FIG. 41a, Table 62), and purification and analysis were performed in the same manner as described in Example 1. Expression, purification and analysis were performed for EGM02, EGM03, EGM04, EGM05, and EGM06 in the same manner as mentioned above (Table 62).

TABLE 62

| | | SEQ ID |
|---|---|---|
| Name | Sequence | NO |
| EGM01 HC | QVQLKQSGPGLVQPSQSLSITCTVSGFSLT | 590 |
| | NYGVHWVRQSPGKCLEWLGVIWSGGNTDYN | |
| | TPFTSRLSINKDNSKSQVFFKMNSLQSNDT | |
| | AIYYCARALTYYDYEFAYWGQGTLVTVSAA | |
| | STKGPSVFPLAPSSKSTSGGTAALGCLVKD | |
| | YFPEPVTVSWNSGALTSGVHTFPAVLQSSG | |
| | LYSLSSVVTVPSSSLGTQTYICNVNHKPSN | |
| | TKVDKKVEPKSCDKTHTCPPCPAPELLGGP | |
| | SVFLFPPKPKDTLMISRTPEVTCVVVDVSH | |
| | EDPEVKFNWYVDGVEVHNAKTKPREEQYNS | |
| | TYRVVSVLTVLHQDWLNGKEYKCKVSNKAL | |
| | PAPIEKTISKAKGQPREPQVYTLPPCREEM | |
| | TKNQVSLWCLVKGFYPSDIAVEWESNGQPE | |
| | NNYKTTPPVLDSDGSFFLYSKLTVDKSRWQ | |
| | QGNVFSCSVMHEALHNHYTQKSLSLSPGK | |
| EGM01 LC | DILLTQSPVILSVSPGERVSFSCRASQSIG | 591 |
| | TNIHWYQQRTNGSPRLLIKYASESISGIPS | |
| | RFSGSGSGTDFTLSINSVESEDIADYYCQQ | |
| | NNNWPTTFGCGTKLELKRTVAAPSVFIFPP | |
| | SDEQLKSGTASVVCLLNNFYPREAKVQWKV | |
| | DNALQSGNSQESVTEQDSKDSTYSLSSTLT | |
| | LSKADYEKHKVYACEVTHQGLSSPVTKSFN | |
| | RGECGGGGSGGGGSGGGGSEPKSSDKTHTC | |
| | PPCPAPELLGGPSVFLFPPKPKDTLMISRT | |
| | PEVTCVVVDVSHEDPEVKFNWYVDGVEVHN | |
| | AKTKPREEQYNSTYRVVSVLTVLHQDWLNG | |
| | KEYKCKVSNKALPAPIEKTISKAKGQPREP | |
| | QVYTLPPCREEMTKNQVSLWCLVKGFYPSD | |
| | IAVEWESNGQPENNYKTTPPVLDSDGSFFL | |
| | YSKLTVDKSRWQQGNVFSCSVMHEALHNHY | |
| | TQKSLSLSPGK | |
| EGM02 HC | QVQLQESGPGLVKPSQTLSLTCTVSGGSIS | 592 |
| | SGDYYWSWIRQPPGKCLEWIGYIYYSGSTD | |
| | YNPSLKSRVTMSVDTSKNQFSLKVNSVTAA | |
| | DTAVYYCARVSIFGVGTFDYWGQGTLVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPELLG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPCRE | |
| | EMTKNQVSLWCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | K | |
| EGM02 LC | EIVMTQSPATLSLSPGERATLSCRASQSVS | 593 |
| | SYLAWYQQKPGQAPRLLIYDASNRATGIPA | |
| | RFSGSGSGTDFTLTISSLEPEDFAVYYCHQ | |
| | YGSTPLTFGCGTKAEIKRTVAAPSVFIFPP | |
| | SDEQLKSGTASVVCLLNNFYPREAKVQWKV | |
| | DNALQSGNSQESVTEQDSKDSTYSLSSTLT | |
| | LSKADYEKHKVYACEVTHQGLSSPVTKSFN | |
| | RGECGGGGSGGGGSGGGGSEPKSSDKTHTC | |
| | PPCPAPELLGGPSVFLFPPKPKDTLMISRT | |
| | PEVTCVVVDVSHEDPEVKFNWYVDGVEVHN | |
| | AKTKPREEQYNSTYRVVSVLTVLHQDWLNG | |
| | KEYKCKVSNKALPAPIEKTISKAKGQPREP | |
| | QVYTLPPCREEMTKNQVSLWCLVKGFYPSD | |
| | IAVEWESNGQPENNYKTTPPVLDSDGSFFL | |
| | YSKLTVDKSRWQQGNVFSCSVMHEALHNHY | |
| | TQKSLSLSPGK | |
| EGM03 HC | QVQLQESGPGLVKPSETLSLTCTVSGGSVS | 594 |
| | SGDYYWTWIRQSPGKCLEWIGHIYYSGNTN | |
| | YNPSLKSRLTISIDTSKTQFSLKLSSVTAA | |
| | DTAIYYCVRDRVTGAFDIWGQGTMVTVSSA | |
| | STKGPSVFPLAPSSKSTSGGTAALGCLVKD | |
| | YFPEPVTVSWNSGALTSGVHTFPAVLQSSG | |
| | LYSLSSVVTVPSSSLGTQTYICNVNHKPSN | |
| | TKVDKKVEPKSCDKTHTCPPCPAPELLGGP | |
| | SVFLFPPKPKDTLMISRTPEVTCVVVDVSH | |
| | EDPEVKFNWYVDGVEVHNAKTKPREEQYNS | |
| | TYRVVSVLTVLHQDWLNGKEYKCKVSNKAL | |
| | PAPIEKTISKAKGQPREPQVYTLPPCREEM | |
| | TKNQVSLWCLVKGFYPSDIAVEWESNGQPE | |
| | NNYKTTPPVLDSDGSFFLYSKLTVDKSRWQ | |
| | QGNVFSCSVMHEALHNHYTQKSLSLSPGK | |
| EGM03 LC | DIQMTQSPSSLSASVGDRVTITCQASQDIS | 595 |
| | NYLNWYQQKPGKAPKLLIYDASNLETGVPS | |
| | RFSGSGSGTDFTFTISSLQPEDIATYFCQH | |
| | FDHLPLAFGCGTKVEIKRTVAAPSVFIFPP | |
| | SDEQLKSGTASVVCLLNNFYPREAKVQWKV | |
| | DNALQSGNSQESVTEQDSKDSTYSLSSTLT | |
| | LSKADYEKHKVYACEVTHQGLSSPVTKSFN | |
| | RGECGGGGSGGGGSGGGGSEPKSSDKTHTC | |
| | PPCPAPELLGGPSVFLFPPKPKDTLMISRT | |
| | PEVTCVVVDVSHEDPEVKFNWYVDGVEVHN | |
| | AKTKPREEQYNSTYRVVSVLTVLHQDWLNG | |
| | KEYKCKVSNKALPAPIEKTISKAKGQPREP | |
| | QVYTLPPCREEMTKNQVSLWCLVKGFYPSD | |
| | IAVEWESNGQPENNYKTTPPVLDSDGSFFL | |
| | YSKLTVDKSRWQQGNVFSCSVMHEALHNHY | |
| | TQKSLSLSPGK | |
| EGM04 HC | QVQLVQSGAEVKKPGASVKVSCKASGYTFT | 596 |
| | SHWMHWVRQAPGQCLEWIGEFNPSNGRTNY | |
| | NEKFKSKATMTVDTSTNTAYMELSSLRSED | |
| | TAVYYCASRDYDYDGRYFDYWGQGTLVTVS | |
| | SASTKGPSVFPLAPSSKSTSGGTAALGCLV | |
| | KDYFPEPVTVSWNSGALTSGVHTFPAVLQS | |
| | SGLYSLSSVVTVPSSSLGTQTYICNVNHKP | |
| | SNTKVDKKVEPKSCDKTHTCPPCPAPELLG | |
| | GPSVFLFPPKPKDTLMISRTPEVTCVVVDV | |
| | SHEDPEVKFNWYVDGVEVHNAKTKPREEQY | |
| | NSTYRVVSVLTVLHQDWLNGKEYKCKVSNK | |
| | ALPAPIEKTISKAKGQPREPQVYTLPPCRE | |
| | EMTKNQVSLWCLVKGFYPSDIAVEWESNGQ | |
| | PENNYKTTPPVLDSDGSFFLYSKLTVDKSR | |
| | WQQGNVFSCSVMHEALHNHYTQKSLSLSPG | |
| | KDIQMTQSPSSLSASVGDRVTITCSASSSV | |
| EGM04 LC | TYMYWYQQKPGKAPKLLI | 597 |
| | YDTSNLASGVPSRFSGSGSGTDYTFTISSL | |
| | QPEDIATYYCQQWSSHIFTFGCGTKVEIKR | |
| | TVAAPSVFIFPPSDEQLKSGTASVVCLLNN | |
| | FYPREAKVQWKVDNALQSGNSQESVTEQDS | |
| | KDSTYSLSSTLTLSKADYEKHKVYACEVTH | |
| | QGLSSPVTKSFNRGECGGGGSGGGGSGGGG | |
| | SEPKSSDKTHTCPPCPAPELLGGPSVFLFP | |
| | PKPKDTLMISRTPEVTCVVVDVSHEDPEVK | |
| | FNWYVDGVEVHNAKTKPREEQYNSTYRVVS | |
| | VLTVLHQDWLNGKEYKCKVSNKALPAPIEK | |
| | TISKAKGQPREPQVYTLPPCREEMTKNQVS | |
| | LWCLVKGFYPSDIAVEWESNGQPENNYKTT | |
| | PPVLDSDGSFFLYSKLTVDKSRWQQGNVFS | |
| | CSVMHEALHNHYTQKSLSLSPGK | |
| EGM05 HC | QVQLVQSGAEVKKPGSSVKVSCKASGFTFT | \|598 |
| | DYKIHWVRQAPGQCLEWMGYFNPNSGYSTY | |
| | AQKFQGRVTITADKSTSTAYMELSSLRSED | |
| | TAVYYCARLSPGGYYVMDAWGQGTTVTVSS | |
| | ASTKGPSVFPLAPSSKSTSGGTAALGCLVK | |
| | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQYN | |
| | STYRVVSVLTVLHQDWLNGKEYKCKVSNKA | |
| | LPAPIEKTISKAKGQPREPQVYTLPPCREE | |
| | MTKNQVSLWCLVKGFYPSDIAVEWESNGQP | |
| | ENNYKTTPPVLDSDGSFFLYSKLTVDKSRW | |
| | QQGNVFSCSVMHEALHNHYTQKSLSLSPGK | |
| EGM05 LC | DIQMTQSPSSLSASVGDRVTITCRASQGIN | \|599 |
| | NYLNWYQQKPGKAPKRLIYNTNNLQTGVPS | |
| | RFSGSGSGTEFTLTISSLQPEDFATYYCLQ | |
| | HNSFPTFGCGTKLEIKRTVAAPSVFIFPPS | |
| | DEQLKSGTASVVCLLNNFYPREAKVQWKVD | |
| | NALQSGNSQESVTEQDSKDSTYSLSSTLTL | |
| | SKADYEKHKVYACEVTHQGLSSPVTKSFNR | |
| | GECGGGGSGGGGSGGGGSEPKSSDKTHTCP | |
| | PCPAPELLGGPSVFLFPPKPKDTLMISRTP | |
| | EVTCVVVDVSHEDPEVKFNWYVDGVEVHNA | |
| | KTKPREEQYNSTYRVVSVLTVLHQDWLNGK | |
| | EYKCKVSNKALPAPIEKTISKAKGQPREPQ | |
| | VYTLPPCREEMTKNQVSLWCLVKGFYPSDI | |
| | AVEWESNGQPENNYKTTPPVLDSDGSFFLY | |
| | SKLTVDKSRWQQGNVFSCSVMHEALHNHYT | |
| | QKSLSLSPGK | |

Table 63 below shows the heavy chain and light chain nucleotide sequences of the engineered antibodies targeting EGFR.

TABLE 63

| Name | Sequence | SEQ ID NO |
|---|---|---|
| EGM01 HC | CAAGTACAGTTGAAACAATCAGGGCCTGGTTTGGTGCAGC | 600 |
| | CTTCCCAATCACTTAGCATCACCTGCACTGTCTCAGGGTT | |
| | CAGTCTTACAAACTACGGCGTACACTGGGTACGGCAAAGC | |
| | CCTGGTAAGTGCCTGGAGTGGCTTGGGGTTATATGGTCTG | |
| | GAGGGAATACCGACTATAACACACCCTTTACCAGCAGGCT | |
| | GTCCATCAATAAAGATAACTCTAAATCCCAGGTCTTCTTT | |
| | AAAATGAACTCCCTCCAGTCTAATGACACTGCCATATATT | |
| | ACTGTGCTAGAGCATTGACTTACTACGATTATGAGTTCGC | |
| | ATATTGGGGACAGGGTACTCTGGTCACCGTATCCGCTGCT | |
| | AGCACCAAAGGACCTAGTGTTTTTCCTCTTGCCCCTTCCT | |
| | CAAAGTCTACCTCTGGGGGGACAGCCGCTCTGGGCTGCCT | |
| | GGTCAAGGATTATTTCCCAGAGCCTGTCACTGTCAGTTGG | |
| | AACTCTGGAGCCTTGACTTCTGGTGTTCATACATTTCCTG | |
| | CTGTCCTTCAAAGCAGCGGCTTGTACTCATTGTCTTCTGT | |
| | TGTGACAGTACCCTCAAGCAGCCTCGGCACTCAGACATAC | |
| | ATCTGCAATGTCAACCACAAACCCTCAAATACAAAGGTAG | |
| | ATAAAAAAGTCGAACCAAAGTCTTGTGACAAAACTCACAC | |
| | GTGCCCACCGTGCCCAGCACCTGAACTCCTGGGGGGACCG | |
| | TCAGTCTTCCTCTTCCCCCCAAAACCCAAGGACACCCTCA | |
| | TGATCTCCCGGACCCCTGAGGTCACATGCGTGGTGGTGGA | |
| | CGTGAGCCACGAAGACCCTGAGGTCAAGTTCAACTGGTAC | |
| | GTGGACGGCGTGGAGGTGCATAATGCCAAGACAAAGCCGC | |
| | GGGAGGAGCAGTACAACAGCACGTACCGTGTGGTCAGCGT | |
| | CCTCACCGTCCTGCACCAGGACTGGCTGAATGGCAAGGAG | |
| | TACAAGTGCAAGGTCTCCAACAAAGCCCTCCCAGCCCCCA | |
| | TCGAGAAAACCATCTCCAAAGCCAAAGGGCAGCCCCGAGA | |
| | ACCACAGGTGTACACCCTGCCCCCATGCCGGGAGGAGATG | |
| | ACCAAGAACCAGGTCAGCCTGTGGTGCCTGGTCAAAGGCT | |
| | TCTATCCCAGCGACATCGCCGTGGAGTGGGAGAGCAATGG | |
| | GCAGCCGGAGAACAACTACAAGACCACGCCTCCCGTGCTG | |
| | GACTCCGACGGCTCCTTCTTCCTCTACAGCAAGCTCACCG | |
| | TGGACAAGAGCAGGTGGCAGCAGGGGAACGTCTTCTCATG | |
| | CTCCGTGATGCATGAGGCTCTGCACAACCACTACACGCAG | |
| | AAGAGCCTCTCCCTGTCTCCGGGTAAA | |
| EGM01 LC | GACATCTTGCTTACTCAATCACCTGTAATACTTTCAGTTT | 601 |
| | CACCAGGTGAACGCGTTAGCTTCTCTTGTAGAGCCTCCCA | |
| | ATCTATAGGTACTAATATCCATTGGTATCAGCAGAGAACC | |
| | AACGGGTCTCCTCGTTTGCTCATTAAATATGCAAGCGAAT | |
| | CAATCTCAGGGATTCCTAGCCGTTTTAGTGGCTCTGGCAG | |
| | TGGTACTGATTTCACACTCAGCATCAATTCTGTAGAGAGC | |
| | GAAGATATTGCAGACTACTATTGCCAACAGAACAATAATT | |
| | GGCCCACAACCTTCGGGTGTGGCACAAAATTGGAACTCAA | |
| | ACGTACGGTGGCAGCTCCCAGCGTTTTTATCTTTCCCCCA | |
| | TCCGACGAGCAGCTCAAGAGTGGCACTGCCTCTGTAGTTT | |
| | GTTTGCTGAATAACTTCTATCCACGTGAAGCAAAAGTACA | |
| | GTGGAAGGTCGATAATGCCCTTCAGAGCGGTAACAGCCAA | |
| | GAAAGTGTTACCGAGCAAGATTCCAAAGATTCCACTTACA | |
| | GTCTGTCCAGCACATTGACACTGAGTAAGGCTGATTACGA | |
| | AAAACACAAGGTGTACGCATGCGAGGTGACACACCAAGGT | |
| | CTTTCATCTCCTGTAACTAAGAGCTTTAACCGGGGAGAAT | |
| | GTGGTGGTGGGGGCAGCGGGGGCGGAGGTAGTGGAGGCGG | |
| | CGGTAGTGAACCAAAGAGTAGTGACAAAACTCACACGTGC | |
| | CCACCGTGCCCAGCACCTGAACTCCTGGGGGGACCGTCAG | |
| | TCTTCCTCTTCCCCCCAAAACCCAAGGACACCCTCATGAT | |
| | CTCCCGGACCCCTGAGGTCACATGCGTGGTGGTGGACGTG | |
| | AGCCACGAAGACCCTGAGGTCAAGTTCAACTGGTACGTGG | |
| | ACGGCGTGGAGGTGCATAATGCCAAGACAAAGCCGCGGGA | |
| | GGAGCAGTACAACAGCACGTACCGTGTGGTCAGCGTCCTC | |
| | ACCGTCCTGCACCAGGACTGGCTGAATGGCAAGGAGTACA | |
| | AGTGCAAGGTCTCCAACAAAGCCCTCCCAGCCCCCATCGA | |
| | GAAAACCATCTCCAAAGCCAAAGGGCAGCCCCGAGAACCA | |
| | CAGGTGTACACCCTGCCCCCATGCCGGGAGGAGATGACCA | |
| | AGAACCAGGTCAGCCTGTGGTGCCTGGTCAAAGGCTTCTA | |
| | TCCCAGCGACATCGCCGTGGAGTGGGAGAGCAATGGGCAG | |
| | CCGGAGAACAACTACAAGACCACGCCTCCCGTGCTGGACT | |
| | CCGACGGCTCCTTCTTCCTCTACAGCAAGCTCACCGTGGA | |
| | CAAGAGCAGGTGGCAGCAGGGGAACGTCTTCTCATGCTCC | |
| | GTGATGCATGAGGCTCTGCACAACCACTACACGCAGAAGA | |
| | GCCTCTCCCTGTCTCCGGGTAAA | |
| EGM02 HC | CAAGTGCAGCTTCAAGAGTCTGGACCAGGGCTGGTCAAGC | 602 |
| | CCTCCCAAACCTTGAGCCTCACCTGTACTGTTTCCGGGGG | |
| | CAGCATAAGTTCTGGTGATTACTACTGGAGTTGGATACGC | |
| | CAACCTCCCGGAAAATGTCTGGAGTGGATTGGGTATATCT | |
| | ATTATAGTGGCTCAACAGACTACAATCCTTCTCTCAAGAG | |
| | TCGGGTAACTATGAGCGTAGATACAAGTAAAAACCAATTT | |
| | TCCCTTAAAGTCAATAGCGTTACAGCCGCTGACACTGCAG | |
| | TTTACTACTGTGCCCGTGTTTCAATCTTCGGTGTCGGCAC | |
| | TTTCGATTACTGGGGTCAGGGTACTCTGGTTACCGTGTCA | |
| | TCCGCTAGCACCAAAGGACCTAGTGTTTTTCCTCTTGCCC | |
| | CTTCCTCAAAGTCTACCTCTGGGGGGACAGCCGCTCTGGG | |
| | CTGCCTGGTCAAGGATTATTTCCCAGAGCCTGTCACTGTC | |
| | AGTTGGAACTCTGGAGCCTTGACTTCTGGTGTTCATACAT | |
| | TTCCTGCTGTCCTTCAAAGCAGCGGCTTGTACTCATTGTC | |
| | TTCTGTTGTGACAGTACCCTCAAGCAGCCTCGGCACTCAG | |
| | ACATACATCTGCAATGTCAACCACAAACCCTCAAATACAA | |
| | AGGTAGATAAAAAAGTCGAACCAAAGTCTTGTGACAAAAC | |
| | TCACACGTGCCCACCGTGCCCAGCACCTGAACTCCTGGGG | |
| | GGACCGTCAGTCTTCCTCTTCCCCCCAAAACCCAAGGACA | |
| | CCCTCATGATCTCCCGGACCCCTGAGGTCACATGCGTGGT | |
| | GGTGGACGTGAGCCACGAAGACCCTGAGGTCAAGTTCAAC | |
| | TGGTACGTGGACGGCGTGGAGGTGCATAATGCCAAGACAA | |
| | AGCCGCGGGAGGAGCAGTACAACAGCACGTACCGTGTGGT | |
| | CAGCGTCCTCACCGTCCTGCACCAGGACTGGCTGAATGGC | |
| | AAGGAGTACAAGTGCAAGGTCTCCAACAAAGCCCTCCCAG | |
| | CCCCCATCGAGAAAACCATCTCCAAAGCCAAAGGGCAGCC | |
| | CCGAGAACCACAGGTGTACACCCTGCCCCCATGCCGGGAG | |
| | GAGATGACCAAGAACCAGGTCAGCCTGTGGTGCCTGGTCA | |
| | AAGGCTTCTATCCCAGCGACATCGCCGTGGAGTGGGAGAG | |
| | CAATGGGCAGCCGGAGAACAACTACAAGACCACGCCTCCC | |
| | GTGCTGGACTCCGACGGCTCCTTCTTCCTCTACAGCAAGC | |
| | TCACCGTGGACAAGAGCAGGTGGCAGCAGGGGAACGTCTT | |
| | CTCATGCTCCGTGATGCATGAGGCTCTGCACAACCACTAC | |
| | ACGCAGAAGAGCCTCTCCCTGTCTCCGGGTAAA | |
| EGM02 LC | GAGATTGTTATGACCCAAAGCCCTGCAACACTTAGTTTGT | 603 |
| | CTCCAGGAGAGCGCGCCACCCTTTCTTGTCGTGCATCCCA | |
| | AAGCGTTAGCAGCTATCTCGCCTGGTATCAGCAGAAACCC | |
| | GGACAGGCTCCACGATTGCTGATCTACGACGCAAGTAATA | |
| | GAGCTACAGGAATACCTGCTCGTTTCTCAGGCTCTGGATC | |
| | TGGCACTGATTTCACCTTGACCATAAGCAGCCTGGAGCCC | |
| | GAGGATTTCGCTGTATATTATTGCCATCAATACGGGAGTA | |
| | CCCCCCTCACATTCGGTTGCGGGACTAAGGCCGAAATTAA | |
| | ACGTACGGTGGCAGCTCCCAGCGTTTTTATCTTTCCCCCA | |
| | TCCGACGAGCAGCTCAAGAGTGGCACTGCCTCTGTAGTTT | |
| | GTTTGCTGAATAACTTCTATCCACGTGAAGCAAAAGTACA | |
| | GTGGAAGGTCGATAATGCCCTTCAGAGCGGTAACAGCCAA | |
| | GAAAGTGTTACCGAGCAAGATTCCAAAGATTCCACTTACA | |
| | GTCTGTCCAGCACATTGACACTGAGTAAGGCTGATTACGA | |
| | AAAACACAAGGTGTACGCATGCGAGGTGACACACCAAGGT | |
| | CTTTCATCTCCTGTAACTAAGAGCTTTAACCGGGGAGAAT | |
| | GTGGTGGTGGGGGCAGCGGGGGCGGAGGTAGTGGAGGCGG | |
| | CGGTAGTGAACCAAAGAGTAGTGACAAAACTCACACGTGC | |
| | CCACCGTGCCCAGCACCTGAACTCCTGGGGGGACCGTCAG | |
| | TCTTCCTCTTCCCCCCAAAACCCAAGGACACCCTCATGAT | |
| | CTCCCGGACCCCTGAGGTCACATGCGTGGTGGTGGACGTG | |
| | AGCCACGAAGACCCTGAGGTCAAGTTCAACTGGTACGTGG | |
| | ACGGCGTGGAGGTGCATAATGCCAAGACAAAGCCGCGGGA | |
| | GGAGCAGTACAACAGCACGTACCGTGTGGTCAGCGTCCTC | |
| | ACCGTCCTGCACCAGGACTGGCTGAATGGCAAGGAGTACA | |
| | AGTGCAAGGTCTCCAACAAAGCCCTCCCAGCCCCCATCGA | |
| | GAAAACCATCTCCAAAGCCAAAGGGCAGCCCCGAGAACCA | |
| | CAGGTGTACACCCTGCCCCCATGCCGGGAGGAGATGACCA | |
| | AGAACCAGGTCAGCCTGTGGTGCCTGGTCAAAGGCTTCTA | |
| | TCCCAGCGACATCGCCGTGGAGTGGGAGAGCAATGGGCAG | |
| | CCGGAGAACAACTACAAGACCACGCCTCCCGTGCTGGACT | |
| | CCGACGGCTCCTTCTTCCTCTACAGCAAGCTCACCGTGGA | |
| | CAAGAGCAGGTGGCAGCAGGGGAACGTCTTCTCATGCTCC | |
| | GTGATGCATGAGGCTCTGCACAACCACTACACGCAGAAGA | |
| | GCCTCTCCCTGTCTCCGGGTAAA | |
| EGM03 HC | CAGGTACAACTTCAAGAAAGTGGTCCAGGTCTTGTAAAAC | 604 |
| | CCTCAGAAACTTTGAGTCTCACTTGTACCGTCAGTGGCGG | |
| | AAGTGTAAGTTCTGGCGATTACTACTGGACCTGGATACGC | |
| | CAGTCTCCAGGCAAATGTCTGGAGTGGATAGGCCACATCT | |
| | ACTACAGCGGGAACACCAATTACAATCCATCTCTTAAATC | |
| | AAGGTTGACAATTTCAATAGACACCAGTAAGACCCAGTTT | |
| | TCTCTCAAACTTAGCAGTGTAACAGCAGCAGATACTGCAA | |
| | TCTACTACTGCGTTAGAGACCGTGTTACAGGGGCTTTCGA | |
| | CATCTGGGGGCAGGGAACTATGGTTACCGTCTCTTCTGCT | |
| | AGCACCAAAGGACCTAGTGTTTTTCCTCTTGCCCCTTCCT | |
| | CAAAGTCTACCTCTGGGGGGACAGCCGCTCTGGGCTGCCT | |
| | GGTCAAGGATTATTTCCCAGAGCCTGTCACTGTCAGTTGG | |
| | AACTCTGGAGCCTTGACTTCTGGTGTTCATACATTTCCTG | |
| | CTGTCCTTCAAAGCAGCGGCTTGTACTCATTGTCTTCTGT | |
| | TGTGACAGTACCCTCAAGCAGCCTCGGCACTCAGACATAC | |
| | ATCTGCAATGTCAACCACAAACCCTCAAATACAAAGGTAG | |
| | ATAAAAAAGTCGAACCAAAGTCTTGTGACAAAACTCACAC | |
| | GTGCCCACCGTGCCCAGCACCTGAACTCCTGGGGGGACCG | |
| | TCAGTCTTCCTCTTCCCCCCAAAACCCAAGGACACCCTCA | |
| | TGATCTCCCGGACCCCTGAGGTCACATGCGTGGTGGTGGA | |
| | CGTGAGCCACGAAGACCCTGAGGTCAAGTTCAACTGGTAC | |
| | GTGGACGGCGTGGAGGTGCATAATGCCAAGACAAAGCCGC | |
| | GGGAGGAGCAGTACAACAGCACGTACCGTGTGGTCAGCGT | |
| | CCTCACCGTCCTGCACCAGGACTGGCTGAATGGCAAGGAG | |
| | TACAAGTGCAAGGTCTCCAACAAAGCCCTCCCAGCCCCCA | |
| | TCGAGAAAACCATCTCCAAAGCCAAAGGGCAGCCCCGAGA | |
| | ACCACAGGTGTACACCCTGCCCCCATGCCGGGAGGAGATG | |
| | ACCAAGAACCAGGTCAGCCTGTGGTGCCTGGTCAAAGGCT | |
| | TCTATCCCAGCGACATCGCCGTGGAGTGGGAGAGCAATGG | |
| | GCAGCCGGAGAACAACTACAAGACCACGCCTCCCGTGCTG | |
| | GACTCCGACGGCTCCTTCTTCCTCTACAGCAAGCTCACCG | |
| | TGGACAAGAGCAGGTGGCAGCAGGGGAACGTCTTCTCATG | |
| | CTCCGTGATGCATGAGGCTCTGCACAACCACTACACGCAG | |
| | AAGAGCCTCTCCCTGTCTCCGGGTAAA | |
| EGM03 LC | GACATACAGATGACACAGAGTCCTAGCTCATTGTCAGCCT | 605 |
| | CTGTCGGTGACCGCGTCACTATCACCTGCCAAGCCAGCCA | |
| | AGACATATCAAATTATCTTAACTGGTACCAGCAAAAGCCT | |
| | GGAAAGGCTCCAAAACTGCTGATTTACGACGCCTCTAATT | |
| | TGGAGACCGGGGTTCCCTCTAGGTTCAGCGGGTCTGGTTC | |
| | AGGCACCGACTTTACATTCACTATCTCAAGTCTCCAGCCA | |
| | GAGGACATCGCTACATACTTTTGCCAACATTTTGACCATC | |
| | TGCCCCTGGCATTTGGGTGTGGCACCAAGGTAGAAATTAA | |
| | GCGTACGGTGGCAGCTCCCAGCGTTTTTATCTTTCCCCCA | |
| | TCCGACGAGCAGCTCAAGAGTGGCACTGCCTCTGTAGTTT | |
| | GTTTGCTGAATAACTTCTATCCACGTGAAGCAAAAGTACA | |
| | GTGGAAGGTCGATAATGCCCTTCAGAGCGGTAACAGCCAA | |
| | GAAAGTGTTACCGAGCAAGATTCCAAAGATTCCACTTACA | |
| | GTCTGTCCAGCACATTGACACTGAGTAAGGCTGATTACGA | |
| | AAAACACAAGGTGTACGCATGCGAGGTGACACACCAAGGT | |
| | CTTTCATCTCCTGTAACTAAGAGCTTTAACCGGGGAGAAT | |
| | GTGGTGGTGGGGGCAGCGGGGGCGGAGGTAGTGGAGGCGG | |
| | CGGTAGTGAACCAAAGAGTAGTGACAAAACTCACACGTGC | |
| | CCACCGTGCCCAGCACCTGAACTCCTGGGGGGACCGTCAG | |
| | TCTTCCTCTTCCCCCCAAAACCCAAGGACACCCTCATGAT | |
| | CTCCCGGACCCCTGAGGTCACATGCGTGGTGGTGGACGTG | |
| | AGCCACGAAGACCCTGAGGTCAAGTTCAACTGGTACGTGG | |
| | ACGGCGTGGAGGTGCATAATGCCAAGACAAAGCCGCGGGA | |
| | GGAGCAGTACAACAGCACGTACCGTGTGGTCAGCGTCCTC | |
| | ACCGTCCTGCACCAGGACTGGCTGAATGGCAAGGAGTACA | |
| | AGTGCAAGGTCTCCAACAAAGCCCTCCCAGCCCCCATCGA | |
| | GAAAACCATCTCCAAAGCCAAAGGGCAGCCCCGAGAACCA | |
| | CAGGTGTACACCCTGCCCCCATGCCGGGAGGAGATGACCA | |
| | AGAACCAGGTCAGCCTGTGGTGCCTGGTCAAAGGCTTCTA | |
| | TCCCAGCGACATCGCCGTGGAGTGGGAGAGCAATGGGCAG | |
| | CCGGAGAACAACTACAAGACCACGCCTCCCGTGCTGGACT | |
| | CCGACGGCTCCTTCTTCCTCTACAGCAAGCTCACCGTGGA | |
| | CAAGAGCAGGTGGCAGCAGGGGAACGTCTTCTCATGCTCC | |
| | GTGATGCATGAGGCTCTGCACAACCACTACACGCAGAAGA | |
| | GCCTCTCCCTGTCTCCGGGTAAA | |
| EGM04 HC | CAAGTCCAACTGGTCCAATCTGGGGCAGAGGTCAAGAAAC | 606 |
| | CTGGCGCAAGCGTAAAGGTATCCTGTAAAGCATCTGGCTA | |
| | CACATTCACTTCACATTGGATGCACTGGGTTCGGCAGGCA | |
| | CCTGGGCAATGTCTTGAATGGATTGGGGAGTTTAACCCCA | |
| | GTAACGGGAGGACTAACTACAATGAAAAGTTCAAGTCCAA | |
| | AGCAACCATGACCGTCGATACCAGCACAAACACTGCCTAC | |
| | ATGGAACTTTCATCATTGCGATCTGAAGACACAGCAGTAT | |
| | ATTACTGTGCCAGTAGGGATTACGACTACGACGGTCGCTA | |
| | CTTCGACTATTGGGGGCAAGGTACTTTGGTAACAGTGAGT | |
| | AGTGCTAGCACCAAAGGACCTAGTGTTTTTCCTCTTGCCC | |
| | CTTCCTCAAAGTCTACCTCTGGGGGGACAGCCGCTCTGGG | |
| | CTGCCTGGTCAAGGATTATTTCCCAGAGCCTGTCACTGTC | |
| | AGTTGGAACTCTGGAGCCTTGACTTCTGGTGTTCATACAT | |
| | TTCCTGCTGTCCTTCAAAGCAGCGGCTTGTACTCATTGTC | |
| | TTCTGTTGTGACAGTACCCTCAAGCAGCCTCGGCACTCAG | |
| | ACATACATCTGCAATGTCAACCACAAACCCTCAAATACAA | |
| | AGGTAGATAAAAAAGTCGAACCAAAGTCTTGTGACAAAAC | |
| | TCACACGTGCCCACCGTGCCCAGCACCTGAACTCCTGGGG | |
| | GGACCGTCAGTCTTCCTCTTCCCCCCAAAACCCAAGGACA | |
| | CCCTCATGATCTCCCGGACCCCTGAGGTCACATGCGTGGT | |
| | GGTGGACGTGAGCCACGAAGACCCTGAGGTCAAGTTCAAC | |
| | TGGTACGTGGACGGCGTGGAGGTGCATAATGCCAAGACAA | |
| | AGCCGCGGGAGGAGCAGTACAACAGCACGTACCGTGTGGT | |
| | CAGCGTCCTCACCGTCCTGCACCAGGACTGGCTGAATGGC | |
| | AAGGAGTACAAGTGCAAGGTCTCCAACAAAGCCCTCCCAG | |
| | CCCCCATCGAGAAAACCATCTCCAAAGCCAAAGGGCAGCC | |
| | CCGAGAACCACAGGTGTACACCCTGCCCCCATGCCGGGAG | |
| | GAGATGACCAAGAACCAGGTCAGCCTGTGGTGCCTGGTCA | |
| | AAGGCTTCTATCCCAGCGACATCGCCGTGGAGTGGGAGAG | |
| | CAATGGGCAGCCGGAGAACAACTACAAGACCACGCCTCCC | |
| | GTGCTGGACTCCGACGGCTCCTTCTTCCTCTACAGCAAGC | |
| | TCACCGTGGACAAGAGCAGGTGGCAGCAGGGGAACGTCTT | |
| | CTCATGCTCCGTGATGCATGAGGCTCTGCACAACCACTAC | |
| | ACGCAGAAGAGCCTCTCCCTGTCTCCGGGTAAA | |
| EGM04 LC | GACATACAGATGACACAATCCCCATCTAGCCTGTCCGCAA | 607 |
| | GTGTTGGGGACCGTGTCACTATAACATGCTCAGCATCATC | |
| | ATCAGTGACTTATATGTACTGGTACCAGCAAAAGCCCGGA | |
| | AAGGCACCTAAACTGCTCATTTATGACACCAGCAATCTTG | |
| | CTTCCGGGGTTCCTTCTCGATTTTCCGGTTCTGGCAGCGG | |
| | TACTGACTATACTTTTACTATCAGTTCTCTGCAACCTGAA | |
| | GACATCGCAACTTACTATTGTCAACAATGGTCCAGCCACA | |
| | TCTTTACTTTTGGATGTGGGACAAAAGTCGAAATTAAACG | |
| | TACGGTGGCAGCTCCCAGCGTTTTTATCTTTCCCCCATCC | |
| | GACGAGCAGCTCAAGAGTGGCACTGCCTCTGTAGTTTGTT | |
| | TGCTGAATAACTTCTATCCACGTGAAGCAAAAGTACAGTG | |
| | GAAGGTCGATAATGCCCTTCAGAGCGGTAACAGCCAAGAA | |
| | AGTGTTACCGAGCAAGATTCCAAAGATTCCACTTACAGTC | |
| | TGTCCAGCACATTGACACTGAGTAAGGCTGATTACGAAAA | |
| | ACACAAGGTGTACGCATGCGAGGTGACACACCAAGGTCTT | |
| | TCATCTCCTGTAACTAAGAGCTTTAACCGGGGAGAATGTG | |
| | GTGGTGGGGGCAGCGGGGGCGGAGGTAGTGGAGGCGGCGG | |
| | TAGTGAACCAAAGAGTAGTGACAAAACTCACACGTGCCCA | |
| | CCGTGCCCAGCACCTGAACTCCTGGGGGGACCGTCAGTCT | |
| | TCCTCTTCCCCCCAAAACCCAAGGACACCCTCATGATCTC | |
| | CCGGACCCCTGAGGTCACATGCGTGGTGGTGGACGTGAGC | |
| | CACGAAGACCCTGAGGTCAAGTTCAACTGGTACGTGGACG | |
| | GCGTGGAGGTGCATAATGCCAAGACAAAGCCGCGGGAGGA | |
| | GCAGTACAACAGCACGTACCGTGTGGTCAGCGTCCTCACC | |
| | GTCCTGCACCAGGACTGGCTGAATGGCAAGGAGTACAAGT | |
| | GCAAGGTCTCCAACAAAGCCCTCCCAGCCCCCATCGAGAA | |
| | AACCATCTCCAAAGCCAAAGGGCAGCCCCGAGAACCACAG | |
| | GTGTACACCCTGCCCCCATGCCGGGAGGAGATGACCAAGA | |
| | ACCAGGTCAGCCTGTGGTGCCTGGTCAAAGGCTTCTATCC | |
| | CAGCGACATCGCCGTGGAGTGGGAGAGCAATGGGCAGCCG | |
| | GAGAACAACTACAAGACCACGCCTCCCGTGCTGGACTCCG | |
| | ACGGCTCCTTCTTCCTCTACAGCAAGCTCACCGTGGACAA | |
| | GAGCAGGTGGCAGCAGGGGAACGTCTTCTCATGCTCCGTG | |
| | ATGCATGAGGCTCTGCACAACCACTACACGCAGAAGAGCC | |
| | TCTCCCTGTCTCCGGGTAAA | |
| EGM05 HC | CAAGTACAACTTGTTCAATCTGGAGCAGAGGTTAAGAAAC | 608 |
| | CCGGATCTTCCGTTAAGGTTAGTTGCAAGGCCTCCGGGTT | |
| | TACTTTCACTGACTATAAAATCCATTGGGTCCGGCAGGCC | |
| | CCTGGGCAGTGTCTTGAATGGATGGGCTACTTCAACCCAA | |
| | ATTCTGGTTATTCCACTTATGCCCAGAAGTTTCAAGGCAG | |
| | GGTCACCATCACTGCCGACAAGTCTACTTCAACCGCCTAT | |
| | ATGGAACTTAGCAGTCTGCGATCAGAGGATACAGCAGTCT | |
| | ACTACTGCGCCAGACTGTCACCCGGCGGTTATTATGTGAT | |
| | GGATGCTTGGGGCCAGGGCACCACTGTTACAGTATCCTCT | |
| | GCTAGCACCAAAGGACCTAGTGTTTTTCCTCTTGCCCCTT | |
| | CCTCAAAGTCTACCTCTGGGGGGACAGCCGCTCTGGGCTG | |
| | CCTGGTCAAGGATTATTTCCCAGAGCCTGTCACTGTCAGT | |
| | TGGAACTCTGGAGCCTTGACTTCTGGTGTTCATACATTTC | |
| | CTGCTGTCCTTCAAAGCAGCGGCTTGTACTCATTGTCTTC | |
| | TGTTGTGACAGTACCCTCAAGCAGCCTCGGCACTCAGACA | |
| | TACATCTGCAATGTCAACCACAAACCCTCAAATACAAAGG | |
| | TAGATAAAAAAGTCGAACCAAAGTCTTGTGACAAAACTCA | |
| | CACGTGCCCACCGTGCCCAGCACCTGAACTCCTGGGGGGA | |
| | CCGTCAGTCTTCCTCTTCCCCCCAAAACCCAAGGACACCC | |
| | TCATGATCTCCCGGACCCCTGAGGTCACATGCGTGGTGGT | |
| | GGACGTGAGCCACGAAGACCCTGAGGTCAAGTTCAACTGG | |
| | TACGTGGACGGCGTGGAGGTGCATAATGCCAAGACAAAGC | |
| | CGCGGGAGGAGCAGTACAACAGCACGTACCGTGTGGTCAG | |
| | CGTCCTCACCGTCCTGCACCAGGACTGGCTGAATGGCAAG | |
| | GAGTACAAGTGCAAGGTCTCCAACAAAGCCCTCCCAGCCC | |
| | CCATCGAGAAAACCATCTCCAAAGCCAAAGGGCAGCCCCG | |
| | AGAACCACAGGTGTACACCCTGCCCCCATGCCGGGAGGAG | |
| | ATGACCAAGAACCAGGTCAGCCTGTGGTGCCTGGTCAAAG | |
| | GCTTCTATCCCAGCGACATCGCCGTGGAGTGGGAGAGCAA | |
| | TGGGCAGCCGGAGAACAACTACAAGACCACGCCTCCCGTG | |
| | CTGGACTCCGACGGCTCCTTCTTCCTCTACAGCAAGCTCA | |
| | CCGTGGACAAGAGCAGGTGGCAGCAGGGGAACGTCTTCTC | |
| | ATGCTCCGTGATGCATGAGGCTCTGCACAACCACTACACG | |
| | CAGAAGAGCCTCTCCCTGTCTCCGGGTAAA | |
| EGM05 LC | GACATTCAGATGACTCAGTCCCCATCCTCTCTGTCTGCCA | 609 |
| | GCGTGGGAGATAGGGTCACCATAACTTGTCGGGCATCCCA | |
| | AGGGATCAATAACTACCTCAATTGGTATCAACAAAAACCT | |
| | GGCAAGGCTCCTAAAAGGCTGATTTATAACACTAACAATC | |
| | TCCAAACCGGGGTGCCAAGTCGCTTTAGTGGGTCAGGGAG | |
| | TGGAACAGAGTTTACTCTTACTATCTCCAGCCTCCAGCCC | |
| | GAGGACTTTGCCACTTACTATTGCCTCCAACACAACTCAT | |
| | TTCCAACATTTGGTTGTGGCACTAAACTTGAAATTAAACG | |
| | TACGGTGGCAGCTCCCAGCGTTTTTATCTTTCCCCCATCC | |
| | GACGAGCAGCTCAAGAGTGGCACTGCCTCTGTAGTTTGTT | |
| | TGCTGAATAACTTCTATCCACGTGAAGCAAAAGTACAGTG | |
| | GAAGGTCGATAATGCCCTTCAGAGCGGTAACAGCCAAGAA | |
| | AGTGTTACCGAGCAAGATTCCAAAGATTCCACTTACAGTC | |
| | TGTCCAGCACATTGACACTGAGTAAGGCTGATTACGAAAA | |
| | ACACAAGGTGTACGCATGCGAGGTGACACACCAAGGTCTT | |
| | TCATCTCCTGTAACTAAGAGCTTTAACCGGGGAGAATGTG | |
| | GTGGTGGGGGCAGCGGGGGCGGAGGTAGTGGAGGCGGCGG | |
| | TAGTGAACCAAAGAGTAGTGACAAAACTCACACGTGCCCA | |
| | CCGTGCCCAGCACCTGAACTCCTGGGGGGACCGTCAGTCT | |
| | TCCTCTTCCCCCCAAAACCCAAGGACACCCTCATGATCTC | |
| | CCGGACCCCTGAGGTCACATGCGTGGTGGTGGACGTGAGC | |
| | CACGAAGACCCTGAGGTCAAGTTCAACTGGTACGTGGACG | |
| | GCGTGGAGGTGCATAATGCCAAGACAAAGCCGCGGGAGGA | |
| | GCAGTACAACAGCACGTACCGTGTGGTCAGCGTCCTCACC | |
| | GTCCTGCACCAGGACTGGCTGAATGGCAAGGAGTACAAGT | |
| | GCAAGGTCTCCAACAAAGCCCTCCCAGCCCCCATCGAGAA | |
| | AACCATCTCCAAAGCCAAAGGGCAGCCCCGAGAACCACAG | |
| | GTGTACACCCTGCCCCCATGCCGGGAGGAGATGACCAAGA | |
| | ACCAGGTCAGCCTGTGGTGCCTGGTCAAAGGCTTCTATCC | |
| | CAGCGACATCGCCGTGGAGTGGGAGAGCAATGGGCAGCCG | |
| | GAGAACAACTACAAGACCACGCCTCCCGTGCTGGACTCCG | |
| | ACGGCTCCTTCTTCCTCTACAGCAAGCTCACCGTGGACAA | |
| | GAGCAGGTGGCAGCAGGGGAACGTCTTCTCATGCTCCGTG | |
| | ATGCATGAGGCTCTGCACAACCACTACACGCAGAAGAGCC | |
| | TCTCCCTGTCTCCGGGTAAA | |

Table 64 below shows the polypeptide sequences of the heavy chain variable region (VH) and light chain variable region (VL) of the engineered antibodies targeting EGFR. Table 65 below shows the nucleotide sequences of the heavy chain variable region (VH) and light chain variable region (VL) of the engineered antibodies targeting EGFR.

TABLE 64

| | | | SEQ ID |
|---|---|---|---|
| Name | | Sequence | NO |
| EGM01 | VH | QVQLKQSGPGLVQPSQSLSITCTVS | 610 |
| | | GFSLTNYGVHWVRQSPGKCLEWLGV | |
| | | IWSGGNTDYNTPFTSRLSINKDNSK | |
| | | SQVFFKMNSLQSNDTAIYYCARALT | |
| | | YYDYEFAYWGQGTLVTVSA | |
| | VL | DILLTQSPVILSVSIGERVSFSCRA | 611 |
| | | SQSIGTNIHWYQQRTNGSPRLLIKY | |
| | | ASESISGIPSRFSGSGSGTDFTLSI | |
| | | NSVESEDIADYYCQQNNNWPTTFGC | |
| | | GTKLELK | |
| EGM02 | VH | QVQLQESGPGLVKPSQTLSLTCTVS | 612 |
| | | GGSISSGDYYWSWIRQPPGKCLEWI | |
| | | GYIYYSGSTDYNPSLKSRVTMSVDT | |
| | | SKNQFSLKVNSVTAADTAVYYCARV | |
| | | SIFGVGTFDYWGQGTLVTVSS | |
| | VL | EIVMTQSPATLSLSPGERATLSCRA | 613 |
| | | SQSVSSYLAWYQQKPGQAPRLLIYD | |
| | | ASNRATGIPARFSGSGSGTDFTLTI | |
| | | SSLEPEDFAVYYCHQYGSTPLTFGC | |
| | | GTKAEIK | |
| EGM03 | VH | QVQLQESGPGLVKPSETLSLTCTVS | 614 |
| | | GGSVSSGDYYWTWIRQSPGKCLEWI | |
| | | GHIYYSGNTNYNPSLKSRLTISIDT | |
| | | SKTQFSLKLSSVTAADTAIYYCVRD | |
| | | RVTGAFDIWGQGTMVTVSS | |
| | VL | DIQMTQSPSSLSASVGDRVTITCQA | 615 |
| | | SQDISNYLNWYQQKPGKAPKLLIYD | |
| | | ASNLETGVPSRFSGSGSGTDFTFTI | |
| | | SSLQPEDIATYFCQHFDHLPLAFGC | |
| | | GTKVEIK | |
| EGM04 | VH | QVQLVQSGAEVKKPGASVKVSCKAS | 616 |
| | | GYTFTSHWMHWVRQAPGQCLEWIGE | |
| | | FNPSNGRTNYNEKFKSKATMTVDTS | |
| | | TNTAVYNELSSLRSEDTAVYYCASR | |
| | | DYDYDGRYFDYWGQGTLVTVSS | |
| | VL | DIQMTQSPSSLSASVGDRVTITCSA | 617 |
| | | SSSVTYMYWYQQKPGKAPKLLIYDT | |
| | | SNLASGVPSRFSGSGSGTDYTFTIS | |
| | | SLQPEDIATTYCQQWSSHIFTFGQG | |
| | | TKVEIK | |
| EGM05 | VH | QVQLVQSGAEVKKPGSSVKVSCKAS | 618 |
| | | GFTFTDVKIHWVRQAPGQCLEWMGY | |
| | | FNPNGSYSTYAQKFQGVTITADKST | |
| | | STAYMELSSLRSEDTAVYYCARLSP | |
| | | GGYYVMDAWGQGTTVTVSS | |
| | VL | DIQMTQSPSSLSASVGDRVTITCRA | 619 |
| | | SQGINNYLNWYQQKPGKAPKRLIYN | |
| | | TNNLQTGVPSRFSGSGSGTEFTLTI | |
| | | SSLQPEDFATYYCLQHNSFPTFGQG | |
| | | TKLEIK | |

TABLE 65

| Name | | Sequence | SEQ ID NO |
|---|---|---|---|
| EGM01 | VH | CAAGTACAGTTGAAACAATCAGGGCCTGGTTTGGTGCAGCCTTCCCAATCACTTAGCATCA | 620 |
| | | CCTGCACTGTCTCAGGGTTCAGTCTTACAAACTACGGCGTACACTGGGTACGGCAAAGCCC | |
| | | TGGTAAGTGCCTGGAGTGGCTTGGGGTTATATGGTCTGGAGGGAATACCGACTATAACACA | |
| | | CCCTTTACCAGCAGGCTGTCCATCAATAAAGATAACTCTAAATCCCAGGTCTTCTTTAAAA | |
| | | TGAACTCCCTCCAGTCTAATGACACTGCCATATATTACTGTGCTAGAGCATTGACTTACTA | |
| | | CCCTTTACCAGCAGGCTGTCCATCAATAAAGATAACTCTAAATCCCAGGTCTTCTTTAAAA | |
| | | TGAACTCCCTCCAGTCTAATGACACTGCCATATATTACTGTGCTAGAGCATTGACTTACTA | |
| | | CGATTATGAGTTCGCATATTGGGGACAGGGTACTCTGGTCACCGTATCCGCT | |
| | VL | GACATCTTGCTTACTCAATCACCTGTAATACTTTCAGTTTCACCAGGTGAACGCGTTAGCT | 621 |
| | | TCTCTTGTAGAGCCTCCCAATCTATAGGTACTAATATCCATTGGTATCAGCAGAGAACCAA | |
| | | CGGGTCTCCTCGTTTGCTCATTAAATATGCAAGCGAATCAATCTCAGGGATTCCTAGCCGT | |
| | | TTTAGTGGCTCTGGCAGTGGTACTGATTTCACACTCAGCATCAATTCTGTAGAGAGCGAAG | |
| | | ATATTGCAGACTACTATTGCCAACAGAACAATAATTGGCCCACAACCTTCGGGTGTGGCAC | |
| | | AAAATTGGAACTCAAA | |
| EMG02 | VH | CAAGTGCAGCTTCAAGAGTCTGGACCAGGGCTGGTCAAGCCCTCCCAAACCTTGAGCCTCA | 622 |
| | | CCTGTACTGTTTCCGGGGGCAGCATAAGTTCTGGTGATTACTACTGGAGTTGGATACGCCA | |
| | | ACCTCCCGGAAAATGTCTGGAGTGGATTGGGTATATCTATTATAGTGGCTCAACAGACTAC | |
| | | AATCCTTCTCTCAAGAGTCGGGTAACTATGAGCGTAGATACAAGTAAAAACCAATTTTCCC | |
| | | TTAAAGTCAATAGCGTTACAGCCGCTGACACTGCAGTTTACTACTGTGCCCGTGTTTCAAT | |
| | | CTTCGGTGTCGGCACTTTCGATTACTGGGGTCAGGGTACTCTGGTTACCGTGTCATCC | |
| | VL | GAGATTGTTATGACCCAAAGCCCTGCAACACTTAGTTTGTCTCCAGGAGAGCGCGCCACCC | 623 |
| | | TTTCTTGTCGTGCATCCCAAAGCGTTAGCAGCTATCTCGCCTGGTATCAGCAGAAACCCGG | |
| | | ACAGGCTCCACGATTGCTGATCTACGACGCAAGTAATAGAGCTACAGGAATACCTGCTCGT | |
| | | TTCTCAGGCTCTGGATCTGGCACTGATTTCACCTTGACCATAAGCAGCCTGGAGCCCGAGG | |
| | | ATTTCGCTGTATATTATTGCCATCAATACGGGAGTACCCCCCTCACATTCGGTTGCGGGAC | |
| | | TAAGGCCGAAATTAAA | |
| EMG03 | VH | GACATACAGATGACACAGAGTCCTAGCTCATTGTCAGCCTCTGTCGGTGACCGCGTCACTA | 624 |
| | | TCACCTGCCAAGCCAGCCAAGACATATCAAATTATCTTAACTGGTACCAGCAAAAGCCTGG | |
| | | AAAGGCTCCAAAACTGCTGATTTACGACGCCTCTAATTTGGAGACCGGGGTTCCCTCTAGG | |
| | | TTCAGCGGGTCTGGTTCAGGCACCGACTTTACATTCACTATCTCAAGTCTCCAGCCAGAGG | |
| | | ACATCGCTACATACTTTTGCCAACATTTTGACCATCTGCCCCTGGCATTTGGGTGTGGCAC | |
| | | CAAGGTAGAAATTAAG | |
| | VL | CAGGTACAACTTCAAGAAAGTGGTCCAGGTCTTGTAAAACCCTCAGAAACTTTGAGTCTCA | 625 |
| | | CTTGTACCGTCAGTGGCGGAAGTGTAAGTTCTGGCGATTACTACTGGACCTGGATACGCCA | |
| | | GTCTCCAGGCAAATGTCTGGAGTGGATAGGCCACATCTACTACAGCGGGAACACCAATTAC | |
| | | AATCCATCTCTTAAATCAAGGTTGACAATTTCAATAGACACCAGTAAGACCCAGTTTTCTC | |
| | | TCAAACTTAGCAGTGTAACAGCAGCAGATACTGCAATCTACTACTGCGTTAGAGACCGTGT | |
| | | TACAGGGGCTTTCGACATCTGGGGGCAGGGAACTATGGTTACCGTCTCTTCT | |
| EGM04 | VH | CAAGTCCAACTGGTCCAATCTGGGGCAGAGGTCAAGAAACCTGGCGCAAGCGTAAAGGTAT | 626 |
| | | CCTGTAAAGCATCTGGCTACACATTCACTTCACATTGGATGCACTGGGTTCGGCAGGCACC | |
| | | TGGGCAATGTCTTGAATGGATTGGGGAGTTTAACCCCAGTAACGGGAGGACTAACTACAAT | |
| | | GAAAAGTTCAAGTCCAAAGCAACCATGACCGTCGATACCAGCACAAACACTGCCTACATGG | |
| | | AACTTTCATCATTGCGATCTGAAGACACAGCAGTATATTACTGTGCCAGTAGGGATTACGA | |
| | | CTACGACGGTCGCTACTTCGACTATTGGGGGCAAGGTACTTTGGTAACAGTGAGTAGT | |
| | VL | GACATACAGATGACACAATCCCCATCTAGCCTGTCCGCAAGTGTTGGGGACCGTGTCACTA | 627 |
| | | TAACATGCTCAGCATCATCATCAGTGACTTATATGTACTGGTACCAGCAAAAGCCCGGAAA | |
| | | GGCACCTAAACTGCTCATTTATGACACCAGCAATCTTGCTTCCGGGGTTCCTTCTCGATTT | |
| | | TCCGGTTCTGGCAGCGGTACTGACTATACTTTTACTATCAGTTCTCTGCAACCTGAAGACA | |
| | | TCGCAACTTACTATTGTCAACAATGGTCCAGCCACATCTTTACTTTTGGATGTGGGACAAA | |
| | | AGTCGAAATTAAA | |
| EMG05 | VH | CAAGTACAACTTGTTCAATCTGGAGCAGAGGTTAAGAAACCCGGATCTTCCGTTAAGGTTA | 628 |
| | | GTTGCAAGGCCTCCGGGTTTACTTTCACTGACTATAAAATCCATTGGGTCCGGCAGGCCCC | |
| | | TGGGCAGTGTCTTGAATGGATGGGCTACTTCAACCCAAATTCTGGTTATTCCACTTATGCC | |
| | | CAGAAGTTTCAAGGCAGGGTCACCATCACTGCCGACAAGTCTACTTCAACCGCCTATATGG | |
| | | AACTTAGCAGTCTGCGATCAGAGGATACAGCAGTCTACTACTGCGCCAGACTGTCACCCGG | |
| | | CGGTTATTATGTGATGGATGCTTGGGGCCAGGGCACCACTGTTACAGTATCCTCT | |
| | VL | GACATTCAGATGACTCAGTCCCCATCCTCTCTGTCTGCCAGCGTGGGAGATAGGGTCACCA | 629 |
| | | TAACTTGTCGGGCATCCCAAGGGATCAATAACTACCTCAATTGGTATCAACAAAAACCTGG | |
| | | CAAGGCTCCTAAAAGGCTGATTTATAACACTAACAATCTCCAAACCGGGGTGCCAAGTCGC | |
| | | TTTAGTGGGTCAGGGAGTGGAACAGAGTTTACTCTTACTATCTCCAGCCTCCAGCCCGAGG | |
| | | ACTTTGCCACTTACTATTGCCTCCAACACAACTCATTTCCAACATTTGGTTGTGGCACTAA | |
| | | ACTTGAAATTAAA | |

Table 66 below shows the heavy chain and light chain CDR sequences of the engineered antibodies targeting EGFR.

TABLE 66

| Name | CDR | Sequence | SEQ ID NO |
|---|---|---|---|
| EGM01 | CDR-H1 | NYGVH | 175 |
| | CDR-H2 | VIWSGGNTDYNTPFTS | 176 |
| | CDR-H3 | ALTYYDYEFAY | 177 |
| | CDR-L1 | RASQSIGTNIH | 178 |
| | CDR-L2 | YASESIS | 179 |
| | CDR-L3 | QQNNNWPTT | 180 |
| EGM02 | CDR-H1 | DYYWS | 187 |
| | CDR-H2 | YIYYSGSTDYNPSLKS | 188 |
| | CDR-H3 | VSIFGVGTFDY | 189 |
| | CDR-L1 | RASQSVSSYLA | 190 |
| | CDR-L2 | DASNRAT | 191 |
| | CDR-L3 | HQYGSTPLT | 192 |
| EGM03 | CDR-H1 | DYYWT | 181 |
| | CDR-H2 | HIYYSGNTNYNPSLKS | 182 |
| | CDR-H3 | DRVTGAFDI | 183 |
| | CDR-L1 | QASQDISNYLN | 184 |
| | CDR-L2 | DASNLET | 185 |
| | CDR-L3 | QHFDHLPLA | 186 |
| EGM04 | CDR-H1 | SHWMH | 630 |
| | CDR-H2 | EFNPSNGRTNYNEKFKS | 631 |
| | CDR-H3 | RDYDYDGRYFDY | 632 |
| | CDR-L1 | SASSSVTYMY | 633 |
| | CDR-L2 | DTSNLAS | 634 |
| | CDR-L3 | QQWSSHIFT | 635 |
| EGM05 | CDR-H1 | DYKIH | 193 |
| | CDR-H2 | YFNPNSGYSTYAQKFQG | 194 |
| | CDR-H3 | LSPGGYYVMDA | 195 |
| | CDR-L1 | RASQGINNYLN | 196 |
| | CDR-L2 | NTNNLQT | 197 |
| | CDR-L3 | LQHNSFPT | 198 |

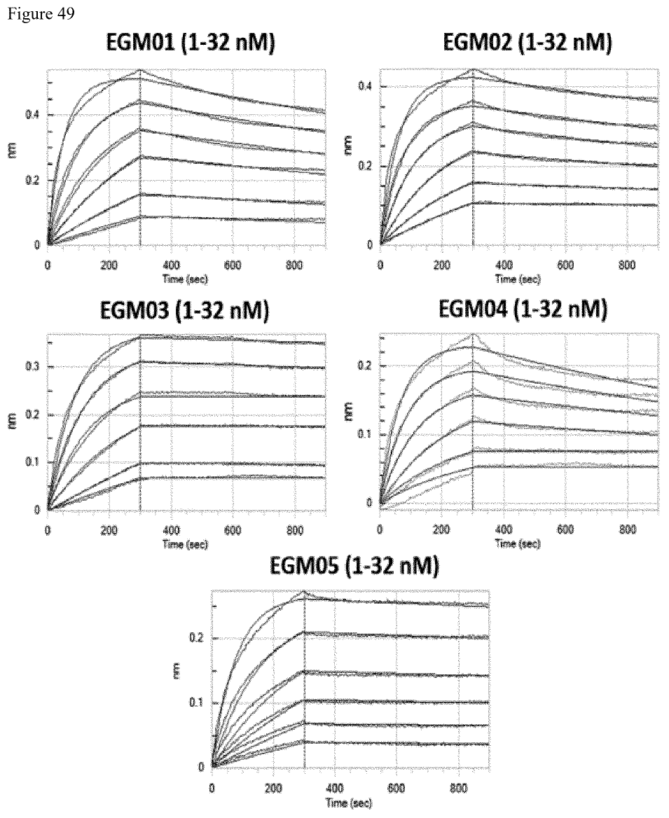

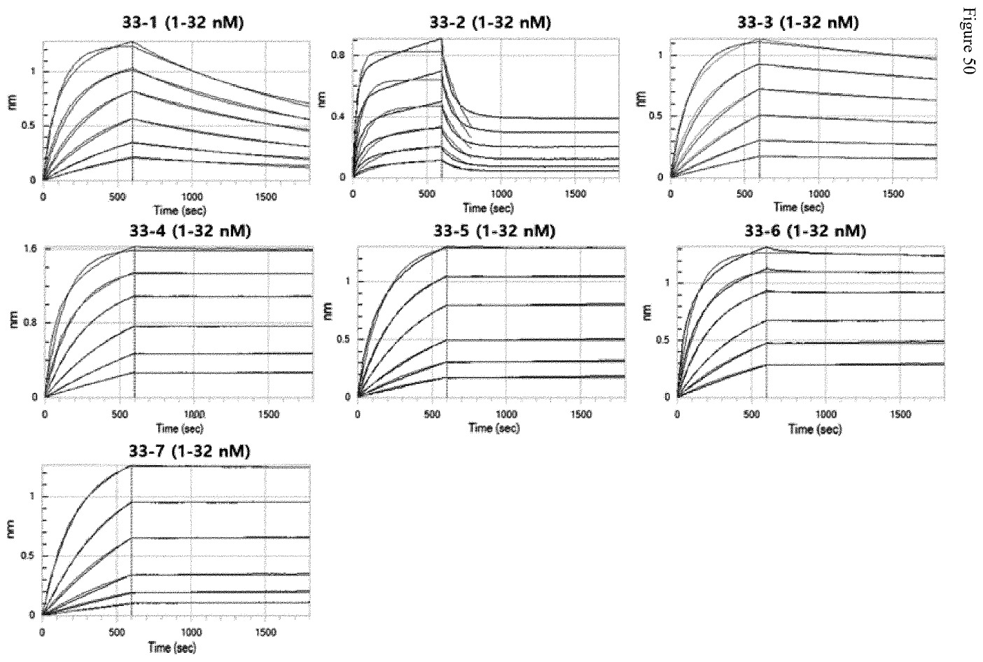

The EGFR protein binding constants of EGM01 to EGM05 were determined using the Octet Red96e (Sartorius). In order to analyze the binding constants of the antibodies, the antibodies were loaded onto the anti-human Fab-CH1 2nd generation (FAB2G) biosensor (Sartorius, 18-5125). Then, the human EGFR recombinant proteins (Sino Biologicals, 10692-H08H) were added in a binding reaction (300 seconds) and a dissociation reaction (600 seconds) at various concentrations (FIG. 49), and the affinities of antibodies for EGFR were calculated (FIG. 49, Table 67). Table 67 below illustrates the binding constants of the engineered antibodies targeting EGFR.

TABLE 67

| Antibodies | K_{D} (nM) | Kₐ (1/Ms) | K_{d} (1/s) |
|---|---|---|---|
| EGM01 | 0.6446 | 6.00E+05 | 3.87E−04 |
| EGM02 | 0.3946 | 7.30E+05 | 2.88E−04 |
| EGM03 | 0.1518 | 4.28E+05 | 6.51E−05 |
| EGM04 | 0.6421 | 6.31E+05 | 4.05E−04 |
| EGM05 | 0.1989 | 4.08E+05 | 8.11E−05 |

### Example 21. Design, Preparation and Analysis of Novel Antibody Targeting CD33

The variant light chain and heavy chain polypeptide sequences of the antibodies that specifically recognize the CD33 protein are shown in Table 68. For GPM01, expression vector consisting of the sequences corresponding to Fc-Hole (SEQ ID NO: 7), 33-1 HC (SEQ ID NO: 636), and 33-1 LC (SEQ ID NO: 637) were co-transfected into EXPICHO-S™ (Gibco, A29127) (FIG. 41a, Table 64), and purification and analysis were performed in the same manner as described in Example 1. Expression, purification and analysis were performed for 33-2, 33-3, 33-4, 33-5, 33-6, and 33-7 in the same manner as mentioned above (FIGS. 41a to 41b, and Tables 68 and Table 69). 33-1, 33-2, and 33-3 bind monovalently to different epitopes of the antigen and have structures consisting of two Fc domains (FIG. 41a). 33-4, 33-5, 33-6, and 33-7 have structures in which the variable regions of the CD33 antibody are linked with a polypeptide linker (SEQ ID NO: 48, SEQ ID NO: 50), and bind biparatopically to CD33, and have two Fc domains (FIG. 41b).

TABLE 68

| Name | Sequence | SEQ ID NO |
|---|---|---|
| 33-1 HC | EVQLVQSGAEVKKPGSSVKVSCKASGYTITDSNIHWVRQAPGQCLEWIGYIYPYNGGTD | 636 |
| | YNQKFKNRATLTVDNPTNTAYEMLSSLRSEDTAFYYCVNGNPWLAYWGQGTLVTVSSAS | |
| | TKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVHNAKTKPREEQY | |
| | NSTRYVVSVLTVLHQDWLNGKEYKCKVSNKALPAPIEKTISKAKGQPREPQVYTLPPCR | |
| | EEMTKNQVSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 33-1 LC | DIQLTQSPSTLSASVGDRVTITCRASESLDNYGIRFLTWFQQKPGKAPKLLMYAASNQG | 637 |
| | SGVPSRFSGSGSGTEFTLTISSLQPDDFATYYCQQTKEVPWSFGCGTKVEVKRTVAAPS | |
| | VFIFPPSDEQLKSGTASVVCLLNNFYPREAKVQWKVDNALQSGNSQESVTEQDSKDSTY | |
| | SLSSTLTLSKADYEKHKVVACEVTHQGLSSPVTKSFNRGECGGGGSGGGGSGGGGSEPK | |
| | SSDKTHTCPPCPAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCKVSNKALPAPIEKTI | |
| | SKAKGQPREPQVYTLPPCREEMTKNQVSLWCLVKGFYPSDIAVEWESNGQPENNYKTTP | |
| | PVLDSDGSFFLYSKLTVDKSRWQQGNVFSCSVMHEALHNHYTQKSLSLSPGK | |
| 33-2 HC | QVQLVQSGAEVKKPGSSVKVSCKASGYTFTDYNMHWVRQAPGQCLEWIGYIYPYNGGTG | 638 |
| | YNQKFNSKATITADESTNTAYMELSSLRSEDTAVYYCARGRPAMDYWGQGTLVTVSSAS | |
| | TKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVHNAKTKPREEQY | |
| | NSTRYVVSVLTVLHQDWLNGKEYKCKVSNKALPAPIEKTISKAKGQPREPQVYTLPPCR | |
| | EEMTKNQVSLWCLVKGFYPSDIAVEWESNGQPENNYKTTPPVLDSDGSFFLYSKLTVDK | |
| | SRWQQGNVFSCSVMHEALHNHYTQKSLSLSPGK | |
| 32-2 LC | DIQMTQSPSSLSASVGDRVTITCRASESVDNYGISFMNWFQQKPGKAPKLLIYAASNQG | 639 |
| | SGVPSRFSGSGSGTDFTLTISSLQPDDFATYYCQQSKEVPWTFGQCTKVEIKRTVAAPS | |
| | VFIFPPSDEQLKSGTASVVCLLNNFYPREAKVQWKVDNALQSGNSQESVTEQDSKDSTY | |
| | SLSSTLTLSKADYEKHKVYACEVTHQGLSSPVTKSFNRGECGGGGSGGGGSGGGGSEPK | |
| | SSDKTHTCPPCPAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCKVSNAKLPAPIEKTI | |
| | SKAKGQPREPQVYTLPPCREEMTKNQVSLWCLVKGFYPSDIAVEWESNGQPENNYKTTP | |
| | PVLDSDGSFFLYSKLTVDKSRWQQGNVFSCSVMHEALHNHYTQKSLSLSPGK | |
| 33-3 HC | QVQLQQPGAEVVKPGASVKMSCKASGYTFTSYYIHWIKQTPGQCLEWVGVIYPGNDDIS | 640 |
| | YNQKFQGKATLTADKSSTTAYMQLSSLTSEDSAVYYCAREVRLRYFDVWGQGTTVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCDKTHTCPPCPAPELL | |
| | GGPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPREPQVYTLPP | |
| | CREEMTKNQVSLWCLVKGFYPSDIAVEWESNGQPENNYKTTPPVLDSDGSFFLYSKLTV | |
| | DKSRWQQGNVFSCSVMHEALHNHYTQKSLSLSPGK | |
| 33-3 LC | EIVLTQSPGSLAVSPGERVTMSCKSSQSVFFSSSQKNYLAWYQQIPGQSPRLLIYWAST | 641 |
| | RESGVPDRFTGSGSGTDFTLTISSVQPEDLAIYYCHQYLSSRTFGCGTKLEIKRTVAAP | |
| | SVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGECGGGGSGGGGSGGGGSEP | |
| | KSSDKTHTCPPCPAPELLGGPSVFLFPPKPKDTLMISRTPEVTCVVVDVSHEDPEVKFN | |
| | WYVDGVEVHNAKTKPREEQYNSTYRVVSVLTVLHQDWLNGKEYKCKVSNKALPAPIEKT | |
| | ISKAKGQPREPQVYTLPPCREEMTKNQVSLWCLVKGFYPSDIAVEWESNGQPENNYKTT | |
| | PPVLDSDGSFFLYSKLTVDKSRWQQGNVFSCSVMHEALHNHYTQKSLSLSPGK | |
| 33-4 HC | EVQLVQSGAEVKKPGSSVKVSCKASGYTITDSNIHWVRQAPGQSLEWIGYIYPYNGGTD | 642 |
| | YNQKFKNRATLTVDNPTNTAYMELSSLRSEDTAFYYCVNGNPWLAYWGQGTLVTVSSAS | |
| | TKGPSVFPLAPQVQLVQSGAEVKKPGSSVKVSCKASGYTFTDYNMHWVRQAPGQCLEWI | |
| | GYIYPYNGGTGYNQKFKSKATITADESTNTAYMELSSLRSEDTAVYYCARGRPAMDYWG | |
| | QGTLVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKTHTC | |
| | PPCPAPELLGGPSVFLFPPKPKDTLMISRTPEVTCVVVDVSHEDPEVKFNWYVDGVEVH | |
| | NAKTKPREEQYNSTRYVVSVLTVLHQDWLNGKEYCKCVSNKALPAPIEKTISKAKGQPR | |
| | EPQVYTLPPCREEMTKNQVSLWCLVKGFYPSDIAVEWESNGQPENNYKTTPPVLDSDGS | |
| | FFLYSKLTVDKSRWQQGNVFSCSVMHEALHNHYTQKSLSLSPGK | |
| 33-4 LC | DIQLTQSPSTLSASVGDRVTITCRASESLDNYGIRFLTWFQQKPGKAPKLLMYAASNQG | 643 |
| | SGVPSRFSGSGSGTEFTLTISSLQPDDFATYYCQQTKEVPWSFGQGTKVEVKRTVAAPS | |
| | VFIFPPDIQMTQSPSSLSASVGDRVTITCRASESVDNYGISFMNWFQQKPGAKPKLLIY | |
| | AASNQGSGVPSRFSGSGSGTDFTLTISSLQPDDFATYYCQQSKEVPWTFGCGTKVEIKR | |
| | TVAAPSVFIFPPSDEQLKSGTASVVCLLNNFYPREAKVQWKVDNALQSGNSQESVTEQD | |
| | SKDSTYSLSSTLTLSKADYEKHKVYACEVTHQGLSSPVTKSFNRGECGGGGSGGGGSGG | |
| | GGSEPKSSDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPA | |
| | PIEKTISKAKGQPREPQVYTLPPCREEMTKNQVSLWCLVKGFYPSDIAVEWESNGQPEN | |
| | NYKTIPPVLDSDGSFFLYSKLTVDKSRWQQGNVFSCSVMHEALHNHYTQKSLSLSPGK | |
| 33-5 HC | QVQLQQPGAEVKKPGASVKMSCKASGYTFTSYYIHWIKQTPGQGLEWVGVIYPGNDDIS | 644 |
| | YNQKFQGKATLTADKSSTTAYMQLSSLTSEDSAVYYCAREVRLRYFDVWGQGTTVTVSS | |
| | ASKTGPSVFPLAPQVQLVQSGAEVKKPGSSVKVSCKASGYTFTDYNMHWVRQAPGQCLE | |
| | WIGYIYPYNGGTGYNQKFKSKATITADESTNTAYMELSSLRSEDTAVYYCARGRPAMDY | |
| | WGQGTLVTVSSASTKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVDKKVEPKSCDKTH | |
| | TCPPCPAPELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTISKAKGQ | |
| | PREPQVYTLPPCREEMTKNQVSLWCLVKGFYPSDIAVEWESNGQPENNYKTTPPVLDSD | |
| | GSFFLYSKLTVDKSRWQQGNVFSCSVMHEALHNHYTQKSLSLSPGK | |
| 33-5 LC | EIVLTQSPGSLAVSPGERVTMSCKSSQSVFFSSSQKNYLAWYQQIFGQSPRLLIYWAST | 645 |
| | RESGVPDRFTGSGSGTDFTLTISSVQPEDLAIYYCHQYLSSRTFGQGTKLEIKRTVAAP | |
| | SVFIFPPDIQMTQSPSSLSASVGDRVTITCRASESVDNYGISFMNWFQQKPGKAPKLLI | |
| | YAASNQGSGVPSRFSGSGSGTDFTLTISSLQPDDFATYYCQQSKEVPWTFGCGTKVEIK | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGECGGGGSGGGGSG | |
| | GGGSEPKSSDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEYKCKVSNKALP | |
| | APIEKTISKAKGQPREPQVYTLPPCREEMTKNQVSLWCLVKGFYPSDIAVEWESNGQPE | |
| | NNYKTTPPVLDSDGSFFLYSKLTVDKSRWQQGNVFSCSVMHEALHNHYTQKSLSLSPGK | |
| 33-6 HC | QVQLVQSGAEVKKPGSSVKVSCKASGYTFTDYNMHWVRQAPGQGLEWIGYIYPYNGGTG | 646 |
| | YNQKFKSKATITADESTNTAYMELSSLRSEDTAVYYCARGRPAMDYWGQGTLVTVSSAS | |
| | TKGPSVFPLAPEVQLVQSGAEVKKPGSSVKVSCKASGYTITDSNIHWVRQAPGQCLEWI | |
| | GYIYPYNGGTDYNQKFKNRATLTVDNPTNTAYMELSSLRSEDTAFYYCVNGNPWLAYWG | |
| | QGTLVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKTHTC | |
| | PPCPAPELLGGPSVFLFPPKPKDTLMISRTPEVTCVVVDVSHEDPEVKFNWYVDGVEVH | |
| | NAKTKPREEQYNSTYRVVSVLTVLHQDWLNGKEYKCKVSNKALPAPIEKTISKAKGQPR | |
| | EPQVYTLPPCREEMTKNQVSLWCLVKGFYPSDIAVEWESNGQPENNYKTTPPVLDSDGS | |
| | FFLYSKLTVDKSRWQQGNVFSCSVMHEALHNHYTQKSLSLSPGK | |
| 33-6 LC | DIQMTQSPSSLSASVGDRVTITCRASESVDNYGISFMNWFQQKPGKAPKLLIYAASNQG | 647 |
| | SGVPSRFSGSGSGTDFTLTISSLQPDDFATYYCQQSKEVPWTFGQGTKVEIKRTVAAPS | |
| | VFIFPPSDEQLKSGTASVVCLLNNFYPREAKVQWKVDNALQSGNSQESVTEQDSKDSTY | |
| | SLSSTLTLSKADYEKHKVYACEVTHQGLSSPVTKSFNRGECGGGGSGGGGSGGGGSEPK | |
| | SSDKTHTCPPCPAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCKVSNKALPAPIEKTI | |
| | SKAKGQPREPQVYTLPPCREEMTKNQVSLWCLVKGFYPSDIAVEWESNGQPENNYKTTP | |
| | PVLDSDGSFFLYSKLTVDKSRWQQGNVFSCSVMHEALHNHYTQKSLSLSPGK | |
| 33-7 HC | QVQLVQSGAEVKKPGSSVTVSCKASGYTFTDYNMHWVRQAPGQGLEWIGYIYPYNGGTG | 648 |
| | YNQKFKSKATITADESTNTAYMELSSLRSEDTAVYYCARGRPAMDYWGQGTLVTVSSAS | |
| | TKGPSVFPLAPQVQLQQPGAEVKKPGASVKMSCKASGYTFTSYYIHWIKQTPGQGLEWV | |
| | GVIYPGNDDISYNQKFQGKATLTADKSSTTAYMQLSSLTSEDSAVYYCAREVRLRYFDV | |
| | WGQGTTVTVSSASTKGPSVFPLAPSSKSTSGGTAALGCLVKDYFPEPVTVSWNSGALTS | |
| | GVHTFPAVLQSSGLYSLSSVVTVPSSSLGTYTYICNVNHKPSNTKVDKKVEPKSCDKTH | |
| | TCPPCPAPELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTISKAKGQ | |
| | PREPQVYTLPPCREEMTKNQVSLWCLVKGFYPSDIAVEWESNGQPENNYKTTPPVLDSD | |
| | GSFFLYSKLTVDKSRWQQGNVFSCSVMHEALHNHYTQKSLSLSPGK | |
| 33-7 LC | DIQMTQSPSSLSASVGDRVTITCRASESVDNYGISFMNWFQQKPGKAPKLLIYAASNQG | 649 |
| | SGVPSRFSGSGSGTDFTLTISSLQPDDFATYYCQQSKEVPWTFGQGTKVEIKRTVAAPS | |
| | VFIFPPEIVLTQSPGSLAVSPGERVTMSCKSSQSVFFSSSQKNYLAWYQQIPGQSPRLL | |
| | IYWASTRESGVPDRFTGSGSGTDFTLTISSVQPEDLAIYYCHQYLSSRTFGCGTKLEIK | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGECGGGGSGGGGSG | |
| | GGGSEPKSSDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEYKCKVSNKALP | |
| | APEIKTISKAKGQPREPQVYTLPPCREEMTKNQVSLWCLVKGFYPSDIAVEWESNGQPE | |
| | NNYKTTPPVLDSDGSFFLYSKLTVDKSRWQQGNVFSCSVMHEALHNHYTQKSLSLSPGK | |

TABLE 69

| Name | Sequence | SEQ ID NO |
|---|---|---|
| 33-1 HC | GAGGTTCAGTTGGTTCAGTCAGGAGCAGAGGTCAAAAAACCTGGAAGCTCTGTCAAAGT | 650 |
| | TAGTTGTAAGGCCAGTGGATACACCATAACCGATTCAAATATACATTGGGTTAGGCAAG | |
| | CACCAGGACAGTGCTTGGAATGGATCGGGTACATCTATCCATATAATGGGGGCACCGAT | |
| | TACAACCAAAAGTTTAAGAATCGCGCCACACTCACTGTTGATAATCCAACCAATACAGC | |
| | ATACATGGAGTTGAGCAGTCTTCGGTCCGAGGACACTGCTTTTTACTATTGTGTGAACG | |
| | GTAACCCATGGTTGGCCTATTGGGGCCAAGGTACACTTGTAACAGTTTCATCTGCTAGC | |
| | ACCAAAGGACCTAGTGTTTTTCCTCTTGCCCCTTCCTCAAAGTCTACCTCTGGGGGGAC | |
| | AGCCGCTCTGGGCTGCCTGGTCAAGGATTATTTCCCAGAGCCTGTCACTGTCAGTTGGA | |
| | ACTCTGGAGCCTTGACTTCTGGTGTTCATACATTTCCTGCTGTCCTTCAAAGCAGCGGC | |
| | TTGTACTCATTGTCTTCTGTTGTGACAGTACCCTCAAGCAGCCTCGGCACTCAGACATA | |
| | CATCTGCAATGTCAACCACAAACCCTCAAATACAAAGGTAGATAAAAAAGTCGAACCAA | |
| | AGTCTTGTGACAAAACTCACACGTGCCCACCGTGCCCAGCACCTGAACTCCTGGGGGGA | |
| | CCGTCAGTCTTCCTCTTCCCCCCAAAACCCAAGGACACCCTCATGATCTCCCGGACCCC | |
| | TGAGGTCACATGCGTGGTGGTGGACGTGAGCCACGAAGACCCTGAGGTCAAGTTCAACT | |
| | GGTACGTGGACGGCGTGGAGGTGCATAATGCCAAGACAAAGCCGCGGGAGGAGCAGTAC | |
| | AACAGCACGTACCGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCATCGAGAAAACCA | |
| | TCTCCAAAGCCAAAGGGCAGCCCCGAGAACCACAGGTGTACACCCTGCCCCCATGCCGG | |
| | GAGGAGATGACCAAGAACCAGGTCAGCCTGTGGTGCCTGGTCAAAGGCTTCTATCCCAG | |
| | CGACATCGCCGTGGAGTGGGAGAGCAATGGGCAGCCGGAGAACAACTACAAGACCACGC | |
| | CTCCCGTGCTGGACTCCGACGGCTCCTTCTTCCTCTACAGCAAGCTCACCGTGGACAAG | |
| | AGCAGGTGGCAGCAGGGGAACGTCTTCTCATGCTCCGTGATGCATGAGGCTCTGCACAA | |
| | CCACTACACGCAGAAGAGCCTCTCCCTGTCTCCGGGTAAA | |
| 33-1 LC | GATATACAACTGACTCAGAGTCCCAGCACTCTCAGTGCAAGTGTAGGCGATAGAGTAAC | 651 |
| | TATAACCTGTCGCGCCTCAGAATCTCTTGATAATTATGGGATCCGATTTCTTACTTGGT | |
| | TTCAGCAAAAGCCTGGTAAAGCTCCTAAATTGCTCATGTATGCCGCCAGTAATCAGGGT | |
| | TCAGGAGTTCCTAGTCGTTTCTCTGGGTCAGGAAGCGGCACAGAATTTACCCTTACAAT | |
| | TTCCAGCCTCCAGCCCGACGATTTCGCCACTTACTATTGCCAACAAACTAAAGAGGTTC | |
| | CTTGGAGTTTTGGGTGTGGCACCAAGGTAGAAGTAAAACGTACGGTGGCAGCTCCCAGC | |
| | GTTTTTATCTTTCCCCCATCCGACGAGCAGCTCAAGAGTGGCACTGCCTCTGTAGTTTG | |
| | TTTGCTGAATAACTTCTATCCACGTGAAGCAAAAGTACAGTGGAAGGTCGATAATGCCC | |
| | TTCAGAGCGGTAACAGCCAAGAAAGTGTTACCGAGCAAGATTCCAAAGATTCCACTTAC | |
| | AGTCTGTCCAGCACATTGACACTGAGTAAGGCTGATTACGAAAAACACAAGGTGTACGC | |
| | ATGCGAGGTGACACACCAAGGTCTTTCATCTCCTGTAACTAAGAGCTTTAACCGGGGAG | |
| | AATGTGGTGGTGGGGGCAGCGGGGGCGGAGGTAGTGGAGGCGGCGGTAGTGAACCAAAG | |
| | AGTAGTGACAAAACTCACACGTGCCCACCGTGCCCAGCACCTGAACTCCTGGGGGGACC | |
| | GTCAGTCTTCCTCTTCCCCCCAAAACCCAAGGACACCCTCATGATCTCCCGGACCCCTG | |
| | AGGTCACATGCGTGGTGGTGGACGTGAGCCACGAAGACCCTGAGGTCAAGTTCAACTGG | |
| | TACGTGGACGGCGTGGAGGTGCATAATGCCAAGACAAAGCCGCGGGAGGAGCAGTACAA | |
| | CAGCACGTACCGTGTGGTCAGCGTCCTCACCGTCCTGCACCAGGACTGGCTGAATGGCA | |
| | AGGAGTACAAGTGCAAGGTCTCCAACAAAGCCCTCCCAGCCCCCATCGAGAAAACCATC | |
| | TCCAAAGCCAAAGGGCAGCCCCGAGAACCACAGGTGTACACCCTGCCCCCATGCCGGGA | |
| | GGAGATGACCAAGAACCAGGTCAGCCTGTGGTGCCTGGTCAAAGGCTTCTATCCCAGCG | |
| | ACATCGCCGTGGAGTGGGAGAGCAATGGGCAGCCGGAGAACAACTACAAGACCACGCCT | |
| | CCCGTGCTGGACTCCGACGGCTCCTTCTTCCTCTACAGCAAGCTCACCGTGGACAAGAG | |
| | CAGGTGGCAGCAGGGGAACGTCTTCTCATGCTCCGTGATGCATGAGGCTCTGCACAACC | |
| | ACTACACGCAGAAGAGCCTCTCCCTGTCTCCGGGTAAA | |
| 33-2 HC | CAGGTACAGCTTGTGCAATCTGGAGCTGAGGTCAAAAAACCAGGAAGTTCTGTAAAGGT | 652 |
| | GTCTTGTAAGGCATCTGGTTACACATTTACCGATTACAACATGCATTGGGTACGTCAAG | |
| | CCCCTGGCCAGTGCCTGGAATGGATCGGATATATATACCCCTACAACGGTGGAACAGGT | |
| | TACAACCAAAAATTCAAAAGCAAGGCTACTATCACTGCCGACGAGAGCACTAACACCGC | |
| | TTACATGGAACTGTCTTCCTTGCGTTCAGAAGACACTGCCGTGTATTATTGTGCTCGGG | |
| | GGCGACCCGCAATGGACTATTGGGGCCAAGGTACATTGGTGACTGTCAGTTCCGCTAGC | |
| | ACCAAAGGACCTAGTGTTTTTCCTCTTGCCCCTTCCTCAAAGTCTACCTCTGGGGGGAC | |
| | AGCCGCTCTGGGCTGCCTGGTCAAGGATTATTTCCCAGAGCCTGTCACTGTCAGTTGGA | |
| | ACTCTGGAGCCTTGACTTCTGGTGTTCATACATTTCCTGCTGTCCTTCAAAGCAGCGGC | |
| | TTGTACTCATTGTCTTCTGTTGTGACAGTACCCTCAAGCAGCCTCGGCACTCAGACATA | |
| | CATCTGCAATGTCAACCACAAACCCTCAAATACAAAGGTAGATAAAAAAGTCGAACCAA | |
| | AGTCTTGTGACAAAACTCACACGTGCCCACCGTGCCCAGCACCTGAACTCCTGGGGGGA | |
| | CCGTCAGTCTTCCTCTTCCCCCCAAAACCCAAGGACACCCTCATGATCTCCCGGACCCC | |
| | TGAGGTCACATGCGTGGTGGTGGACGTGAGCCACGAAGACCCTGAGGTCAAGTTCAACT | |
| | GGTACGTGGACGGCGTGGAGGTGCATAATGCCAAGACAAAGCCGCGGGAGGAGCAGTAC | |
| | AACAGCACGTACCGTGTGGTCAGCGTCCTCACCGTCCTGCACCAGGACTGGCTGAATGG | |
| | CAAGGAGTACAAGTGCAAGGTCTCCAACAAAGCCCTCCCAGCCCCCATCGAGAAAACCA | |
| | TCTCCAAAGCCAAAGGGCAGCCCCGAGAACCACAGGTGTACACCCTGCCCCCATGCCGG | |
| | GAGGAGATGACCAAGAACCAGGTCAGCCTGTGGTGCCTGGTCAAAGGCTTCTATCCCAG | |
| | CGACATCGCCGTGGAGTGGGAGAGCAATGGGCAGCCGGAGAACAACTACAAGACCACGC | |
| | CTCCCGTGCTGGACTCCGACGGCTCCTTCTTCCTCTACAGCAAGCTCACCGTGGACAAG | |
| | AGCAGGTGGCAGCAGGGGAACGTCTTCTCATGCTCCGTGATGCATGAGGCTCTGCACAA | |
| | CCACTACACGCAGAAGAGCCTCTCCCTGTCTCCGGGTAAA | |
| 33-2 LC | GACATCCAAATGACACAGTCCCCTAGCTCACTTTCAGCAAGCGTGGGGGACCGAGTTAC | 653 |
| | TATCACATGCCGCGCAAGTGAGTCTGTGGACAACTATGGAATATCATTCATGAACTGGT | |
| | TCCAGCAGAAACCTGGGAAAGCACCCAAGCTGCTTATCTACGCAGCAAGTAATCAGGGT | |
| | AGTGGCGTCCCTTCTCGATTCAGTGGGAGCGGTAGCGGCACCGACTTCACCCTTACCAT | |
| | CTCATCACTTCAACCCGATGATTTTGCTACCTACTATTGCCAGCAATCCAAGGAAGTTC | |
| | CTTGGACCTTCGGGTGTGGGACAAAGGTAGAGATTAAACGTACGGTGGCAGCTCCCAGC | |
| | GTTTTTATCTTTCCCCCATCCGACGAGCAGCTCAAGAGTGGCACTGCCTCTGTAGTTTG | |
| | TTTGCTGAATAACTTCTATCCACGTGAAGCAAAAGTACAGTGGAAGGTCGATAATGCCC | |
| | TTCAGAGCGGTAACAGCCAAGAAAGTGTTACCGAGCAAGATTCCAAAGATTCCACTTAC | |
| | AGTCTGTCCAGCACATTGACACTGAGTAAGGCTGATTACGAAAAACACAAGGTGTACGC | |
| | ATGCGAGGTGACACACCAAGGTCTTTCATCTCCTGTAACTAAGAGCTTTAACCGGGGAG | |
| | AATGTGGTGGTGGGGGCAGCGGGGGCGGAGGTAGTGGAGGCGGCGGTAGTGAACCAAAG | |
| | AGTAGTGACAAAACTCACACGTGCCCACCGTGCCCAGCACCTGAACTCCTGGGGGGACC | |
| | GTCAGTCTTCCTCTTCCCCCCAAAACCCAAGGACACCCTCATGATCTCCCGGACCCCTG | |
| | AGGTCACATGCGTGGTGGTGGACGTGAGCCACGAAGACCCTGAGGTCAAGTTCAACTGG | |
| | TACGTGGACGGCGTGGAGGTGCATAATGCCAAGACAAAGCCGCGGGAGGAGCAGTACAA | |
| | CAGCACGTACCGTGTGGTCAGCGTCCTCACCGTCCTGCACCAGGACTGGCTGAATGGCA | |
| | AGGAGTACAAGTGCAAGGTCTCCAACAAAGCCCTCCCAGCCCCCATCGAGAAAACCATC | |
| | TCCAAAGCCAAAGGGCAGCCCCGAGAACCACAGGTGTACACCCTGCCCCCATGCCGGGA | |
| | GGAGATGACCAAGAACCAGGTCAGCCTGTGGTGCCTGGTCAAAGGCTTCTATCCCAGCG | |
| | ACATCGCCGTGGAGTGGGAGAGCAATGGGCAGCCGGAGAACAACTACAAGACCACGCCT | |
| | CCCGTGCTGGACTCCGACGGCTCCTTCTTCCTCTACAGCAAGCTCACCGTGGACAAGAG | |
| | CAGGTGGCAGCAGGGGAACGTCTTCTCATGCTCCGTGATGCATGAGGCTCTGCACAACC | |
| | ACTACACGCAGAAGAGCCTCTCCCTGTCTCCGGGTAAA | |
| 33-3 HC | CAAGTCCAACTTCAGCAGCCTGGAGCTGAGGTAGTGAAACCCGGCGCATCTGTAAAAAT | 654 |
| | GAGCTGCAAAGCATCAGGTTACACATTTACATCCTACTACATCCATTGGATCAAGCAAA | |
| | CACCAGGCCAATGTCTTGAGTGGGTTGGCGTCATTTACCCAGGAAACGATGATATATCT | |
| | TACAATCAGAAATTTCAAGGGAAAGCCACACTTACAGCCGACAAGAGTTCCACAACTGC | |
| | ATATATGCAACTCTCCTCCCTGACATCTGAAGACAGTGCCGTATACTATTGTGCTCGTG | |
| | AAGTCAGGCTCAGATACTTTGACGTGTGGGGTCAAGGCACAACCGTCACCGTCAGTAGC | |
| | GCTAGCACCAAAGGACCTAGTGTTTTTCCTCTTGCCCCTTCCTCAAAGTCTACCTCTGG | |
| | GGGGACAGCCGCTCTGGGCTGCCTGGTCAAGGATTATTTCCCAGAGCCTGTCACTGTCA | |
| | GTTGGAACTCTGGAGCCTTGACTTCTGGTGTTCATACATTTCCTGCTGTCCTTCAAAGC | |
| | AGCGGCTTGTACTCATTGTCTTCTGTTGTGACAGTACCCTCAAGCAGCCTCGGCACTCA | |
| | GACATACATCTGCAATGTCAACCACAAACCCTCAAATACAAAGGTAGATAAAAAAGTCG | |
| | AACCAAAGTCTTGTGACAAAACTCACACGTGCCCACCGTGCCCAGCACCTGAACTCCTG | |
| | GGGGGACCGTCAGTCTTCCTCTTCCCCCCAAAACCCAAGGACACCCTCATGATCTCCCG | |
| | GACCCCTGAGGTCACATGCGTGGTGGTGGACGTGAGCCACGAAGACCCTGAGGTCAAGT | |
| | TCAACTGGTACGTGGACGGCGTGGAGGTGCATAATGCCAAGACAAAGCCGCGGGAGGAG | |
| | CAGTACAACAGCACGTACCGTGTGGTCAGCGTCCTCACCGTCCTGCACCAGGACTGGCT | |
| | GAATGGCAAGGAGTACAAGTGCAAGGTCTCCAACAAAGCCCTCCCAGCCCCCATCGAGA | |
| | AAACCATCTCCAAAGCCAAAGGGCAGCCCCGAGAACCACAGGTGTACACCCTGCCCCCA | |
| | TGCCGGGAGGAGATGACCAAGAACCAGGTCAGCCTGTGGTGCCTGGTCAAAGGCTTCTA | |
| | TCCCAGCGACATCGCCGTGGAGTGGGAGAGCAATGGGCAGCCGGAGAACAACTACAAGA | |
| | CCACGCCTCCCGTGCTGGACTCCGACGGCTCCTTCTTCCTCTACAGCAAGCTCACCGTG | |
| | GACAAGAGCAGGTGGCAGCAGGGGAACGTCTTCTCATGCTCCGTGATGCATGAGGCTCT | |
| | GCACAACCACTACACGCAGAAGAGCCTCTCCCTGTCTCCGGGTAAA | |
| 33-3 LC | GAGATAGTCCTCACCCAATCACCAGGTAGTCTGGCTGTTAGCCCAGGTGAGCGAGTTAC | 655 |
| | TATGTCTTGCAAGTCCTCTCAATCTGTGTTCTTTTCATCAAGTCAGAAGAATTATCTTG | |
| | CCTGGTATCAGCAGATTCCTGGTCAATCTCCCCGACTGCTTATTTACTGGGCTTCAACT | |
| | CGGGAGTCAGGGGTTCCCGACCGATTCACAGGGTCTGGGTCAGGAACTGACTTTACCCT | |
| | TACTATCAGCTCTGTGCAGCCTGAAGACCTCGCAATATATTATTGTCATCAGTACCTCT | |
| | CTTCTCGCACTTTTGGATGTGGCACCAAATTGGAGATTAAGCGTACGGTGGCAGCTCCC | |
| | AGCGTTTTTATCTTTCCCCCATCCGACGAGCAGCTCAAGAGTGGCACTGCCTCTGTAGT | |
| | TTGTTTGCTGAATAACTTCTATCCACGTGAAGCAAAAGTACAGTGGAAGGTCGATAATG | |
| | CCCTTCAGAGCGGTAACAGCCAAGAAAGTGTTACCGAGCAAGATTCCAAAGATTCCACT | |
| | TACAGTCTGTCCAGCACATTGACACTGAGTAAGGCTGATTACGAAAAACACAAGGTGTA | |
| | CGCATGCGAGGTGACACACCAAGGTCTTTCATCTCCTGTAACTAAGAGCTTTAACCGGG | |
| | GAGAATGTGGTGGTGGGGGCAGCGGGGGCGGAGGTAGTGGAGGCGGCGGTAGTGAACCA | |
| | AAGAGTAGTGACAAAACTCACACGTGCCCACCGTGCCCAGCACCTGAACTCCTGGGGGG | |
| | ACCGTCAGTCTTCCTCTTCCCCCCAAAACCCAAGGACACCCTCATGATCTCCCGGACCC | |
| | CTGAGGTCACATGCGTGGTGGTGGACGTGAGCCACGAAGACCCTGAGGTCAAGTTCAAC | |
| | TGGTACGTGGACGGCGTGGAGGTGCATAATGCCAAGACAAAGCCGCGGGAGGAGCAGTA | |
| | CAACAGCACGTACCGTGTGGTCAGCGTCCTCACCGTCCTGCACCAGGACTGGCTGAATG | |
| | GCAAGGAGTACAAGTGCAAGGTCTCCAACAAAGCCCTCCCAGCCCCCATCGAGAAAACC | |
| | ATCTCCAAAGCCAAAGGGCAGCCCCGAGAACCACAGGTGTACACCCTGCCCCCATGCCG | |
| | GGAGGAGATGACCAAGAACCAGGTCAGCCTGTGGTGCCTGGTCAAAGGCTTCTATCCCA | |
| | GCGACATCGCCGTGGAGTGGGAGAGCAATGGGCAGCCGGAGAACAACTACAAGACCACG | |
| | CCTCCCGTGCTGGACTCCGACGGCTCCTTCTTCCTCTACAGCAAGCTCACCGTGGACAA | |
| | GAGCAGGTGGCAGCAGGGGAACGTCTTCTCATGCTCCGTGATGCATGAGGCTCTGCACA | |
| | ACCACTACACGCAGAAGAGCCTCTCCCTGTCTCCGGGTAAA | |
| 33-4 HC | GAGGTTCAGTTGGTTCAGTCAGGAGCAGAGGTCAAAAAACCTGGAAGCTCTGTCAAAGT | 656 |
| | TAGTTGTAAGGCCAGTGGATACACCATAACCGATTCAAATATACATTGGGTTAGGCAAG | |
| | CACCAGGCACAGTCTTGGAATGGATCGGGTACATCTATCCATATAATGGGGGCACCGAT | |
| | TACAACCAAAAGTTTAAGAATCGCGCCACACTCACTGTTGATAATCCAACCAATACAGC | |
| | ATACATGGAGTTGAGCAGTCTTCGGTCCGAGGACACTGCTTTTTACTATTGTGTGAACG | |
| | GTAACCCATGGTTGGCCTATTGGGGCCAAGGTACACTTGTAACAGTTTCATCCGCTAGC | |
| | ACCAAAGGACCTAGTGTTTTTCCTCTTGCCCCTCAGGTACAGCTTGTGCAATCTGGAGC | |
| | TGAGGTCAAAAAACCAGGAAGTTCTGTAAAGGTGTCTTGTAAGGCATCTGGTTACACAT | |
| | TTACCGATTACAACATGCATTGGGTACGTCAAGCCCCTGGCCAGTGCCTGGAATGGATC | |
| | GGATATATATACCCCTACAACGGTGGAACAGGTTACAACCAAAAATTCAAAAGCAAGGC | |
| | TACTATCACTGCCGACGAGAGCACTAACACCGCTTACATGGAACTGTCTTCCTTGCGTT | |
| | CAGAAGACACTGCCGTGTATTATTGTGCTCGGGGGCGACCCGCAATGGACTATTGGGGC | |
| | CAAGGTACATTGGTGACTGTCAGTTCCGCTAGCACCAAAGGACCTAGTGTTTTTCCTCT | |
| | TGCCCCTTCCTCAAAGTCTACCTCTGGGGGGACAGCCGCTCTGGGCTGCCTGGTCAAGG | |
| | ATTATTTCCCAGAGCCTGTCACTGTCAGTTGGAACTCTGGAGCCTTGACTTCTGGTGTT | |
| | CATACATTTCCTGCTGTCCTTCAAAGCAGCGGCTTGTACTCATTGTCTTCTGTTGTGAC | |
| | AGTACCCTCAAGCAGCCTCGGCACTCAGACATACATCTGCAATGTCAACCACAAACCCT | |
| | CAAATACAAAGGTAGATAAAAAAGTCGAACCAAAGTCTTGTGACAAAACTCACACGTGC | |
| | CCACCGTGCCCAGCACCTGAACTCCTGGGGGGACCGTCAGTCTTCCTCTTCCCCCCAAA | |
| | ACCCAAGGACACCCTCATGATCTCCCGGACCCCTGAGGTCACATGCGTGGTGGTGGACG | |
| | TGAGCCACGAAGACCCTGAGGTCAAGTTCAACTGGTACGTGGACGGCGTGGAGGTGCAT | |
| | AATGCCAAGACAAAGCCGCGGGAGGAGCAGTACAACAGCACGTACCGTGTGGTCAGCGT | |
| | CCTCACCGTCCTGCACCAGGACTGGCTGAATGGCAAGGAGTACAAGTGCAAGGTCTCCA | |
| | ACAAAGCCCTCCCAGCCCCCATCGAGAAAACCATCTCCAAAGCCAAAGGGCAGCCCCGA | |
| | GAACCACAGGTGTACACCCTGCCCCCATGCCGGGAGGAGATGACCAAGAACCAGGTCAG | |
| | CCTGTGGTGCCTGGTCAAAGGCTTCTATCCCAGCGACATCGCCGTGGAGTGGGAGAGCA | |
| | ATGGGCAGCCGGAGAACAACTACAAGACCACGCCTCCCGTGCTGGACTCCGACGGCTCC | |
| | TTCTTCCTCTACAGCAAGCTCACCGTGGACAAGAGCAGGTGGCAGCAGGGGAACGTCTT | |
| | CTCATGCTCCGTGATGCATGAGGCTCTGCACAACCACTACACGCAGAAGAGCCTCTCCC | |
| | TGTCTCCGGGTAAA | |
| 33-4 LC | GATATACAACTGACTCAGAGTCCCAGCACTCTCAGTGCAAGTGTAGGCGATAGAGTAAC | 657 |
| | TATAACCTGTCGCGCCTCAGAATCTCTTGATAATTATGGGATCCGATTTCTTACTTGGT | |
| | TTCAGCAAAAGCCTGGTAAAGCTCCTAAATTGCTCATGTATGCCGCCAGTAATCAGGGT | |
| | TCAGGAGTTCCTAGTCGTTTCTCTGGGTCAGGAAGCGGCACAGAATTTACCCTTACAAT | |
| | TTCCAGCCTCCAGCCCGACGATTTCGCCACTTACTATTGCCAACAAACTAAAGAGGTTC | |
| | CTTGGAGTTTTGGGCAAGGCACCAAGGTAGAAGTAAAACGTACGGTGGCCGCTCCCTCC | |
| | GTTTTTATCTTTCCCCCAGACATCCAAATGACACAGTCCCCTAGCTCACTTTCAGCAAG | |
| | CGTGGGGGACCGAGTTACTATCACATGCCGCGCAAGTGAGTCTGTGGACAACTATGGAA | |
| | TATCATTCATGAACTGGTTCCAGCAGAAACCTGGGAAAGCACCCAAGCTGCTTATCTAC | |
| | GCAGCAAGTAATCAGGGTAGTGGCGTCCCTTCTCGATTCAGTGGGAGCGGTAGCGGCAC | |
| | CGACTTCACCCTTACCATCTCATCACTTCAACCCGATGATTTTGCTACCTACTATTGCC | |
| | AGCAATCCAAGGAAGTTCCTTGGACCTTCGGGTGTGGGACAAAGGTAGAGATTAAACGT | |
| | ACGGTGGCAGCTCCCAGCGTTTTTATCTTTCCCCCATCCGACGAGCAGCTCAAGAGTGG | |
| | CACTGCCTCTGTAGTTTGTTTGCTGAATAACTTCTATCCACGTGAAGCAAAAGTACAGT | |
| | GGAAGGTCGATAATGCCCTTCAGAGCGGTAACAGCCAAGAAAGTGTTACCGAGCAAGAT | |
| | TCCAAAGATTCCACTTACAGTCTGTCCAGCACATTGACACTGAGTAAGGCTGATTACGA | |
| | AAAACACAAGGTGTACGCATGCGAGGTGACACACCAAGGTCTTTCATCTCCTGTAACTA | |
| | AGAGCTTTAACCGGGGAGAATGTGGTGGTGGGGGCAGCGGGGGCGGAGGTAGTGGAGGC | |
| | GGCGGTAGTGAACCAAAGAGTAGTGACAAAACTCACACGTGCCCACCGTGCCCAGCACC | |
| | TGAACTCCTGGGGGGACCGTCAGTCTTCCTCTTCCCCCCAAAACCCAAGGACACCCTCA | |
| | TGATCTCCCGGACCCCTGAGGTCACATGCGTGGTGGTGGACGTGAGCCACGAAGACCCT | |
| | GAGGTCAAGTTCAACTGGTACGTGGACGGCGTGGAGGTGCATAATGCCAAGACAAAGCC | |
| | GCGGGAGGAGCAGTACAACAGCACGTACCGTGTGGTCAGCGTCCTCACCGTCCTGCACC | |
| | AGGACTGGCTGAATGGCAAGGAGTACAAGTGCAAGGTCTCCAACAAAGCCCTCCCAGCC | |
| | CCCATCGAGAAAACCATCTCCAAAGCCAAAGGGCAGCCCCGAGAACCACAGGTGTACAC | |
| | CCTGCCCCCATGCCGGGAGGAGATGACCAAGAACCAGGTCAGCCTGTGGTGCCTGGTCA | |
| | AAGGCTTCTATCCCAGCGACATCGCCGTGGAGTGGGAGAGCAATGGGCAGCCGGAGAAC | |
| | AACTACAAGACCACGCCTCCCGTGCTGGACTCCGACGGCTCCTTCTTCCTCTACAGCAA | |
| | GCTCACCGTGGACAAGAGCAGGTGGCAGCAGGGGAACGTCTTCTCATGCTCCGTGATGC | |
| | ATGAGGCTCTGCACAACCACTACACGCAGAAGAGCCTCTCCCTGTCTCCGGGTAAA | |
| 33-5 HC | CAAGTCCAACTTCAGCAGCCTGGAGCTGAGGTAGTGAAACCCGGCGCATCTGTAAAAAT | 658 |
| | GAGCTGCAAAGCATCAGGTTACACATTTACATCCTACTACATCCATTGGATCAAGCAAA | |
| | CACCAGGCCAAGGTCTTGAGTGGGTTGGCGTCATTTACCCAGGAAACGATGATATATCT | |
| | TACAATCAGAAATTTCAAGGGAAAGCCACACTTACAGCCGACAAGAGTTCCACAACTGC | |
| | ATATATGCAACTCTCCTCCCTGACATCTGAAGACAGTGCCGTATACTATTGTGCTCGTG | |
| | AAGTCAGGCTCAGATACTTTGACGTGTGGGGTCAAGGCACAACCGTCACCGTCAGTAGC | |
| | GCTAGCACCAAAGGACCTAGTGTTTTTCCTCTTGCCCCTCAGGTACAGCTTGTGCAATC | |
| | TGGAGCTGAGGTCAAAAAACCAGGAAGTTCTGTAAAGGTGTCTTGTAAGGCATCTGGTT | |
| | ACACATTTACCGATTACAACATGCATTGGGTACGTCAAGCCCCTGGCCAGTGCCTGGAA | |
| | TGGATCGGATATATATACCCCTACAACGGTGGAACAGGTTACAACCAAAAATTCAAAAG | |
| | CAAGGCTACTATCACTGCCGACGAGAGCACTAACACCGCTTACATGGAACTGTCTTCCT | |
| | TGCGTTCAGAAGACACTGCCGTGTATTATTGTGCTCGGGGGCGACCCGCAATGGACTAT | |
| | TGGGGCCAAGGTACATTGGTGACTGTCAGTTCCGCTAGCACCAAAGGACCTAGTGTTTT | |
| | TCCTCTTGCCCCTTCCTCAAAGTCTACCTCTGGGGGGACAGCCGCTCTGGGCTGCCTGG | |
| | TCAAGGATTATTTCCCAGAGCCTGTCACTGTCAGTTGGAACTCTGGAGCCTTGACTTCT | |
| | GGTGTTCATACATTTCCTGCTGTCCTTCAAAGCAGCGGCTTGTACTCATTGTCTTCTGT | |
| | TGTGACAGTACCCTCAAGCAGCCTCGGCACTCAGACATACATCTGCAATGTCAACCACA | |
| | AACCCTCAAATACAAAGGTAGATAAAAAAGTCGAACCAAAGTCTTGTGACAAAACTCAC | |
| | ACGTGCCCACCGTGCCCAGCACCTGAACTCCTGGGGGGACCGTCAGTCTTCCTCTTCCC | |
| | CCCAAAACCCAAGGACACCCTCATGATCTCCCGGACCCCTGAGGTCACATGCGTGGTGG | |
| | TGGACGTGAGCCACGAAGACCCTGAGGTCAAGTTCAACTGGTACGTGGACGGCGTGGAG | |
| | GTGCATAATGCCAAGACAAAGCCGCGGGAGGAGCAGTACAACAGCACGTACCGTGTGGT | |
| | CAGCGTCCTCACCGTCCTGCACCAGGACTGGCTGAATGGCAAGGAGTACAAGTGCAAGG | |
| | TCTCCAACAAAGCCCTCCCAGCCCCCATCGAGAAAACCATCTCCAAAGCCAAAGGGCAG | |
| | CCCCGAGAACCACAGGTGTACACCCTGCCCCCATGCCGGGAGGAGATGACCAAGAACCA | |
| | GGTCAGCCTGTGGTGCCTGGTCAAAGGCTTCTATCCCAGCGACATCGCCGTGGAGTGGG | |
| | AGAGCAATGGGCAGCCGGAGAACAACTACAAGACCACGCCTCCCGTGCTGGACTCCGAC | |
| | GGCTCCTTCTTCCTCTACAGCAAGCTCACCGTGGACAAGAGCAGGTGGCAGCAGGGGAA | |
| | CGTCTTCTCATGCTCCGTGATGCATGAGGCTCTGCACAACCACTACACGCAGAAGAGCC | |
| | TCTCCCTGTCTCCGGGTAAA | |
| 33-5 LC | GAGATAGTCCTCACCCAATCACCAGGTAGTCTGGCTGTTAGCCCAGGTGAGCGAGTTAC | 659 |
| | TATGTCTTGCAAGTCCTCTCAATCTGTGTTCTTTTCATCAAGTCAGAAGAATTATCTTG | |
| | CCTGGTATCAGCAGATTCCTGGTCAATCTCCCCGACTGCTTATTTACTGGGCTTCAACT | |
| | CGGGAGTCAGGGGTTCCCGACCGATTCACAGGGTCTGGGTCAGGAACTGACTTTACCCT | |
| | TACTATCAGCTCTGTGCAGCCTGAAGACCTCGCAATATATTATTGTCATCAGTACCTCT | |
| | CTTCTCGCACTTTTGGACAGGGCACCAAATTGGAGATTAAGCGTACGGTGGCCGCTCCC | |
| | TCCGTTTTTATCTTTCCCCCAGACATCCAAATGACACAGTCCCCTAGCTCACTTTCAGC | |
| | AAGCGTGGGGGACCGAGTTACTATCACATGCCGCGCAAGTGAGTCTGTGGACAACTATG | |
| | GAATATCATTCATGAACTGGTTCCAGCAGAAACCTGGGAAAGCACCCAAGCTGCTTATC | |
| | TACGCAGCAAGTAATCAGGGTAGTGGCGTCCCTTCTCGATTCAGTGGGAGCGGTAGCGG | |
| | CACCGACTTCACCCTTACCATCTCATCACTTCAACCCGATGATTTTGCTACCTACTATT | |
| | GCCAGCAATCCAAGGAAGTTCCTTGGACCTTCGGGTGTGGGACAAAGGTAGAGATTAAA | |
| | CGTACGGTGGCAGCTCCCAGCGTTTTTATCTTTCCCCCATCCGACGAGCAGCTCAAGAG | |
| | TGGCACTGCCTCTGTAGTTTGTTTGCTGAATAACTTCTATCCACGTGAAGCAAAAGTAC | |
| | AGTGGAAGGTCGATAATGCCCTTCAGAGCGGTAACAGCCAAGAAAGTGTTACCGAGCAA | |
| | GATTCCAAAGATTCCACTTACAGTCTGTCCAGCACATTGACACTGAGTAAGGCTGATTA | |
| | CGAAAAACACAAGGTGTACGCATGCGAGGTGACACACCAAGGTCTTTCATCTCCTGTAA | |
| | CTAAGAGCTTTAACCGGGGAGAATGTGGTGGTGGGGGCAGCGGGGGCGGAGGTAGTGGA | |
| | GGCGGCGGTAGTGAACCAAAGAGTAGTGACAAAACTCACACGTGCCCACCGTGCCCAGC | |
| | ACCTGAACTCCTGGGGGGACCGTCAGTCTTCCTCTTCCCCCCAAAACCCAAGGACACCC | |
| | TCATGATCTCCCGGACCCCTGAGGTCACATGCGTGGTGGTGGACGTGAGCCACGAAGAC | |
| | CCTGAGGTCAAGTTCAACTGGTACGTGGACGGCGTGGAGGTGCATAATGCCAAGACAAA | |
| | GCCGCGGGAGGAGCAGTACAACAGCACGTACCGTGTGGTCAGCGTCCTCACCGTCCTGC | |
| | ACCAGGACTGGCTGAATGGCAAGGAGTACAAGTGCAAGGTCTCCAACAAAGCCCTCCCA | |
| | GCCCCCATCGAGAAAACCATCTCCAAAGCCAAAGGGCAGCCCCGAGAACCACAGGTGTA | |
| | CACCCTGCCCCCATGCCGGGAGGAGATGACCAAGAACCAGGTCAGCCTGTGGTGCCTGG | |
| | TCAAAGGCTTCTATCCCAGCGACATCGCCGTGGAGTGGGAGAGCAATGGGCAGCCGGAG | |
| | AACAACTACAAGACCACGCCTCCCGTGCTGGACTCCGACGGCTCCTTCTTCCTCTACAG | |
| | CAAGCTCACCGTGGACAAGAGCAGGTGGCAGCAGGGGAACGTCTTCTCATGCTCCGTGA | |
| | TGCATGAGGCTCTGCACAACCACTACACGCAGAAGAGCCTCTCCCTGTCTCCGGGTAAA | |
| 33-6 HC | CAGGTACAGCTTGTGCAATCTGGAGCTGAGGTCAAAAAACCAGGAAGTTCTGTAAAGGT | 660 |
| | GTCTTGTAAGGCATCTGGTTACACATTTACCGATTACAACATGCATTGGGTACGTCAAG | |
| | CCCCTGGCCAGGGCCTGGAATGGATCGGATATATATACCCCTACAACGGTGGAACAGGT | |
| | TACAACCAAAAATTCAAAAGCAAGGCTACTATCACTGCCGACGAGAGCACTAACACCGC | |
| | TTACATGGAACTGTCTTCCTTGCGTTCAGAAGACACTGCCGTGTATTATTGTGCTCGGG | |
| | GGCGACCCGCAATGGACTATTGGGGCCAAGGTACATTGGTGACTGTCAGTTCCGCTAGC | |
| | ACCAAAGGACCTAGTGTTTTTCCTCTTGCCCCTGAGGTTCAGTTGGTTCAGTCAGGAGC | |
| | AGAGGTCAAAAAACCTGGAAGCTCTGTCAAAGTTAGTTGTAAGGCCAGTGGATACACCA | |
| | TAACCGATTCAAATATACATTGGGTTAGGCAAGCACCAGGACAGTGCTTGGAATGGATC | |
| | GGGTACATCTATCCATATAATGGGGGCACCGATTACAACCAAAAGTTTAAGAATCGCGC | |
| | CACACTCACTGTTGATAATCCAACCAATACAGCATACATGGAGTTGAGCAGTCTTCGGT | |
| | CCGAGGACACTGCTTTTTACTATTGTGTGAACGGTAACCCATGGTTGGCCTATTGGGGC | |
| | CAAGGTACACTTGTAACAGTTTCATCTGCTAGCACCAAAGGACCTAGTGTTTTTCCTCT | |
| | TGCCCCTTCCTCAAAGTCTACCTCTGGGGGGACAGCCGCTCTGGGCTGCCTGGTCAAGG | |
| | ATTATTTCCCAGAGCCTGTCACTGTCAGTTGGAACTCTGGAGCCTTGACTTCTGGTGTT | |
| | CATACATTTCCTGCTGTCCTTCAAAGCAGCGGCTTGTACTCATTGTCTTCTGTTGTGAC | |
| | AGTACCCTCAAGCAGCCTCGGCACTCAGACATACATCTGCAATGTCAACCACAAACCCT | |
| | CAAATACAAAGGTAGATAAAAAAGTCGAACCAAAGTCTTGTGACAAAACTCACACGTGC | |
| | CCACCGTGCCCAGCACCTGAACTCCTGGGGGGACCGTCAGTCTTCCTCTTCCCCCCAAA | |
| | ACCCAAGGACACCCTCATGATCTCCCGGACCCCTGAGGTCACATGCGTGGTGGTGGACG | |
| | TGAGCCACGAAGACCCTGAGGTCAAGTTCAACTGGTACGTGGACGGCGTGGAGGTGCAT | |
| | AATGCCAAGACAAAGCCGCGGGAGGAGCAGTACAACAGCACGTACCGTGTGGTCAGCGT | |
| | CCTCACCGTCCTGCACCAGGACTGGCTGAATGGCAAGGAGTACAAGTGCAAGGTCTCCA | |
| | ACAAAGCCCTCCCAGCCCCCATCGAGAAAACCATCTCCAAAGCCAAAGGGCAGCCCCGA | |
| | GAACCACAGGTGTACACCCTGCCCCCATGCCGGGAGGAGATGACCAAGAACCAGGTCAG | |
| | CCTGTGGTGCCTGGTCAAAGGCTTCTATCCCAGCGACATCGCCGTGGAGTGGGAGAGCA | |
| | ATGGGCAGCCGGAGAACAACTACAAGACCACGCCTCCCGTGCTGGACTCCGACGGCTCC | |
| | TTCTTCCTCTACAGCAAGCTCACCGTGGACAAGAGCAGGTGGCAGCAGGGGAACGTCTT | |
| | CTCATGCTCCGTGATGCATGAGGCTCTGCACAACCACTACACGCAGAAGAGCCTCTCCC | |
| | TGTCTCCGGGTAAA | |
| 33-6 LC | GACATCCAAATGACACAGTCCCCTAGCTCACTTTCAGCAAGCGTGGGGGACCGAGTTAC | 661 |
| | TATCACATGCCGCGCAAGTGAGTCTGTGGACAACTATGGAATATCATTCATGAACTGGT | |
| | TCCAGCAGAAACCTGGGAAAGCACCCAAGCTGCTTATCTACGCAGCAAGTAATCAGGGT | |
| | AGTGGCGTCCCTTCTCGATTCAGTGGGAGCGGTAGCGGCACCGACTTCACCCTTACCAT | |
| | CTCATCACTTCAACCCGATGATTTTGCTACCTACTATTGCCAGCAATCCAAGGAAGTTC | |
| | CTTGGACCTTCGGGCAAGGGACAAAGGTAGAGATTAAACGTACGGTGGCAGCTCCCAGC | |
| | GTTTTTATCTTTCCCCCATCCGACGAGCAGCTCAAGAGTGGCACTGCCTCTGTAGTTTG | |
| | TTTGCTGAATAACTTCTATCCACGTGAAGCAAAAGTACAGTGGAAGGTCGATAATGCCC | |
| | TTCAGAGCGGTAACAGCCAAGAAAGTGTTACCGAGCAAGATTCCAAAGATTCCACTTAC | |
| | AGTCTGTCCAGCACATTGACACTGAGTAAGGCTGATTACGAAAAACACAAGGTGTACGC | |
| | ATGCGAGGTGACACACCAAGGTCTTTCATCTCCTGTAACTAAGAGCTTTAACCGGGGAG | |
| | AATGTGGTGGTGGGGGCAGCGGGGGCGGAGGTAGTGGAGGCGGCGGTAGTGAACCAAAG | |
| | AGTAGTGACAAAACTCACACGTGCCCACCGTGCCCAGCACCTGAACTCCTGGGGGGACC | |
| | GTCAGTCTTCCTCTTCCCCCCAAAACCCAAGGACACCCTCATGATCTCCCGGACCCCTG | |
| | AGGTCACATGCGTGGTGGTGGACGTGAGCCACGAAGACCCTGAGGTCAAGTTCAACTGG | |
| | TACGTGGACGGCGTGGAGGTGCATAATGCCAAGACAAAGCCGCGGGAGGAGCAGTACAA | |
| | CAGCACGTACCGTGTGGTCAGCGTCCTCACCGTCCTGCACCAGGACTGGCTGAATGGCA | |
| | AGGAGTACAAGTGCAAGGTCTCCAACAAAGCCCTCCCAGCCCCCATCGAGAAAACCATC | |
| | TCCAAAGCCAAAGGGCAGCCCCGAGAACCACAGGTGTACACCCTGCCCCCATGCCGGGA | |
| | GGAGATGACCAAGAACCAGGTCAGCCTGTGGTGCCTGGTCAAAGGCTTCTATCCCAGCG | |
| | ACATCGCCGTGGAGTGGGAGAGCAATGGGCAGCCGGAGAACAACTACAAGACCACGCCT | |
| | CCCGTGCTGGACTCCGACGGCTCCTTCTTCCTCTACAGCAAGCTCACCGTGGACAAGAG | |
| | CAGGTGGCAGCAGGGGAACGTCTTCTCATGCTCCGTGATGCATGAGGCTCTGCACAACC | |
| | ACTACACGCAGAAGAGCCTCTCCCTGTCTCCGGGTAAA | |
| 33-7 HC | CAGGTACAGCTTGTGCAATCTGGAGCTGAGGTCAAAAAACCAGGAAGTTCTGTAAAGGT | 662 |
| | GTCTTGTAAGGCATCTGGTTACACATTTACCGATTACAACATGCATTGGGTACGTCAAG | |
| | CCCCTGGCCAGGGCCTGGAATGGATCGGATATATATACCCCTACAACGGTGGAACAGGT | |
| | TACAACCAAAAATTCAAAAGCAAGGCTACTATCACTGCCGACGAGAGCACTAACACCGC | |
| | TTACATGGAACTGTCTTCCTTGCGTTCAGAAGACACTGCCGTGTATTATTGTGCTCGGG | |
| | GGCGACCCGCAATGGACTATTGGGGCCAAGGTACATTGGTGACTGTCAGTTCCGCTAGC | |
| | ACCAAAGGACCTAGTGTTTTTCCTCTTGCCCCTCAAGTCCAACTTCAGCAGCCTGGAGC | |
| | TGAGGTAGTGAAACCCGGCGCATCTGTAAAAATGAGCTGCAAAGCATCAGGTTACACAT | |
| | TTACATCCTACTACATCCATTGGATCAAGCAAACACCAGGCCAATGTCTTGAGTGGGTT | |
| | GGCGTCATTTACCCAGGAAACGATGATATATCTTACAATCAGAAATTTCAAGGGAAAGC | |
| | CACACTTACAGCCGACAAGAGTTCCACAACTGCATATATGCAACTCTCCTCCCTGACAT | |
| | CTGAAGACAGTGCCGTATACTATTGTGCTCGTGAAGTCAGGCTCAGATACTTTGACGTG | |
| | TGGGGTCAAGGCACAACCGTCACCGTCAGTAGCGCTAGCACCAAAGGACCTAGTGTTTT | |
| | TCCTCTTGCCCCTTCCTCAAAGTCTACCTCTGGGGGGACAGCCGCTCTGGGCTGCCTGG | |
| | TCAAGGATTATTTCCCAGAGCCTGTCACTGTCAGTTGGAACTCTGGAGCCTTGACTTCT | |
| | GGTGTTCATACATTTCCTGCTGTCCTTCAAAGCAGCGGCTTGTACTCATTGTCTTCTGT | |
| | TGTGACAGTACCCTCAAGCAGCCTCGGCACTCAGACATACATCTGCAATGTCAACCACA | |
| | AACCCTCAAATACAAAGGTAGATAAAAAAGTCGAACCAAAGTCTTGTGACAAAACTCAC | |
| | ACGTGCCCACCGTGCCCAGCACCTGAACTCCTGGGGGGACCGTCAGTCTTCCTCTTCCC | |
| | CCCAAAACCCAAGGACACCCTCATGATCTCCCGGACCCCTGAGGTCACATGCGTGGTGG | |
| | TGGACGTGAGCCACGAAGACCCTGAGGTCAAGTTCAACTGGTACGTGGACGGCGTGGAG | |
| | GTGCATAATGCCAAGACAAAGCCGCGGGAGGAGCAGTACAACAGCACGTACCGTGTGGT | |
| | CAGCGTCCTCACCGTCCTGCACCAGGACTGGCTGAATGGCAAGGAGTACAAGTGCAAGG | |
| | TCTCCAACAAAGCCCTCCCAGCCCCCATCGAGAAAACCATCTCCAAAGCCAAAGGGCAG | |
| | CCCCGAGAACCACAGGTGTACACCCTGCCCCCATGCCGGGAGGAGATGACCAAGAACCA | |
| | GGTCAGCCTGTGGTGCCTGGTCAAAGGCTTCTATCCCAGCGACATCGCCGTGGAGTGGG | |
| | AGAGCAATGGGCAGCCGGAGAACAACTACAAGACCACGCCTCCCGTGCTGGACTCCGAC | |
| | GGCTCCTTCTTCCTCTACAGCAAGCTCACCGTGGACAAGAGCAGGTGGCAGCAGGGGAA | |
| | CGTCTTCTCATGCTCCGTGATGCATGAGGCTCTGCACAACCACTACACGCAGAAGAGCC | |
| | TCTCCCTGTCTCCGGGTAAA | |
| 33-7 LC | GACATCCAAATGACACAGTCCCCTAGCTCACTTTCAGCAAGCGTGGGGGACCGAGTTAC | 663 |
| | TATCACATGCCGCGCAAGTGAGTCTGTGGACAACTATGGAATATCATTCATGAACTGGT | |
| | TCCAGCAGAAACCTGGGAAAGCACCCAAGCTGCTTATCTACGCAGCAAGTAATCAGGGT | |
| | AGTGGCGTCCCTTCTCGATTCAGTGGGAGCGGTAGCGGCACCGACTTCACCCTTACCAT | |
| | CTCATCACTTCAACCCGATGATTTTGCTACCTACTATTGCCAGCAATCCAAGGAAGTTC | |
| | CTTGGACCTTCGGGCAAGGGACAAAGGTAGAGATTAAACGTACGGTGGCCGCTCCCTCC | |
| | GTTTTTATCTTTCCCCCAGAGATAGTCCTCACCCAATCACCAGGTAGTCTGGCTGTTAG | |
| | CCCAGGTGAGCGAGTTACTATGTCTTGCAAGTCCTCTCAATCTGTGTTCTTTTCATCAA | |
| | GTCAGAAGAATTATCTTGCCTGGTATCAGCAGATTCCTGGTCAATCTCCCCGACTGCTT | |
| | ATTTACTGGGCTTCAACTCGGGAGTCAGGGGTTCCCGACCGATTCACAGGGTCTGGGTC | |
| | AGGAACTGACTTTACCCTTACTATCAGCTCTGTGCAGCCTGAAGACCTCGCAATATATT | |
| | ATTGTCATCAGTACCTCTCTTCTCGCACTTTTGGATGTGGCACCAAATTGGAGATTAAG | |
| | CGTACGGTGGCAGCTCCCAGCGTTTTTATCTTTCCCCCATCCGACGAGCAGCTCAAGAG | |
| | TGGCACTGCCTCTGTAGTTTGTTTGCTGAATAACTTCTATCCACGTGAAGCAAAAGTAC | |
| | AGTGGAAGGTCGATAATGCCCTTCAGAGCGGTAACAGCCAAGAAAGTGTTACCGAGCAA | |
| | GATTCCAAAGATTCCACTTACAGTCTGTCCAGCACATTGACACTGAGTAAGGCTGATTA | |
| | CGAAAAACACAAGGTGTACGCATGCGAGGTGACACACCAAGGTCTTTCATCTCCTGTAA | |
| | CTAAGAGCTTTAACCGGGGAGAATGTGGTGGTGGGGGCAGCGGGGGCGGAGGTAGTGGA | |
| | GGCGGCGGTAGTGAACCAAAGAGTAGTGACAAAACTCACACGTGCCCACCGTGCCCAGC | |
| | ACCTGAACTCCTGGGGGGACCGTCAGTCTTCCTCTTCCCCCCAAAACCCAAGGACACCC | |
| | TCATGATCTCCCGGACCCCTGAGGTCACATGCGTGGTGGTGGACGTGAGCCACGAAGAC | |
| | CCTGAGGTCAAGTTCAACTGGTACGTGGACGGCGTGGAGGTGCATAATGCCAAGACAAA | |
| | GCCGCGGGAGGAGCAGTACAACAGCACGTACCGTGTGGTCAGCGTCCTCACCGTCCTGC | |
| | ACCAGGACTGGCTGAATGGCAAGGAGTACAAGTGCAAGGTCTCCAACAAAGCCCTCCCA | |
| | GCCCCCATCGAGAAAACCATCTCCAAAGCCAAAGGGCAGCCCCGAGAACCACAGGTGTA | |
| | CACCCTGCCCCCATGCCGGGAGGAGATGACCAAGAACCAGGTCAGCCTGTGGTGCCTGG | |
| | TCAAAGGCTTCTATCCCAGCGACATCGCCGTGGAGTGGGAGAGCAATGGGCAGCCGGAG | |
| | AACAACTACAAGACCACGCCTCCCGTGCTGGACTCCGACGGCTCCTTCTTCCTCTACAG | |
| | CAAGCTCACCGTGGACAAGAGCAGGTGGCAGCAGGGGAACGTCTTCTCATGCTCCGTGA | |
| | TGCATGAGGCTCTGCACAACCACTACACGCAGAAGAGCCTCTCCCTGTCTCCGGGTAAA | |

Table 70 below shows the polypeptide sequences of the heavy chain variable region (VH) and light chain variable region (VL) of the engineered antibodies targeting CD33. Table 71 below shows the nucleotide sequences of the heavy chain variable region (VH) and light chain variable region (VL) of the engineered antibodies targeting CD33.

TABLE 70

| Name | | Sequence | SEQ ID NO |
|---|---|---|---|
| 33-1 | VH | EVQLVQSGAEVKKFGSSVKVSCKASGYTITDSNIHWVRQAPGQCLEWIGYIYPYNGGTDYN | 664 |
| | | QKFKNRATLTVDNPTNTAYMELSSLRSEDTAFYYCVNGNFWLAYWGQGTLVTVSS | |
| | VL | DIQLTQSPSTLSASVGDRVTITCRASESLDNYGIRFLTWFQQKPGKAPKLLMYAASNQGSG | 665 |
| | | VPSRFSGSGSGTEFTLTISSLQPDDFATYYCQQTKEVPWSFGCGTKVEIK | |
| 33-2 | VH | QVQLVQSGAEVKKPGSSVKVSCKASGYTFTDYNMHWVRQAPGQCLEWIGYIYPYNGGTGYN | 666 |
| | | QKFKSKATITADESTNTAYMELSSLRSEDTAVYYCARGRPAMDYWGQGTLVTVSS | |
| | VL | DIQMTQSPSSLSASVGDRVTITCRASESVDNYGISFMNWFQQKPGKAPKLLIYAASNQGSG | 667 |
| | | VPSRFSGSGSGTDFTLTISSLQPDDFATYYCQQSKEVPWTFGCGTKVEIK | |
| 33-3 | VH | QVQLQQPGAEVVKPGASVKMSCKASGYTFTSYYIHWIKQTPGQCLEWVGVIYPGNDDISYN | 668 |
| | | QKFQGKATLTADKSSTTAYMQLSSLTSEDSAVYYCAREVRLRYFDVWGQGTTVTVSS | |
| | VL | EIVLTQSPGSLAVSPGERVTMSCKSSQSVFFSSSQKNYLAWYQQIPGQSPRLLIYWASTRE | 669 |
| | | SGVPDRFTGSGSGTDFTLTISSVQPEDLAIYYCHQYLSSRTFGCGTKLEIK | |
| 33-4 | VH1 | EVQLVQSGAEVKKPGSSVKVSCKASGYTITDSNIHWVRQAPGQSLEWIGYIYPYNGGTDYN | 670 |
| | | QKFKNRATLTVDNPTNTAYMELSSLRSEDTAFYYCVNGNPWLAYWGQGTLVTVSS | |
| | VH2 | QVQLVQSGAEVKKPGSSVKVSCKASGYTFTDYNMHWVRQAPGQCLEWIGYIYPYNGGTGYN | 666 |
| | | QKFKSKATITADESTNTAYMELSSLRSEDTAVYYCARGRPAMDYWGQGLTVTVSS | |
| | VL1 | DIQLTQSPSTLSASVGDRVTITCRASESLDNYGIRFLTWFQQKPGKAPKLLMYAASNQGSG | 671 |
| | | VPSRFSGSGSGTEFTLTISSLQPDDFATYYCQQTKEVPWSFGQGTKVEVK | |
| | VL2 | DIQMTQSPSSLSASVGDRVTITCRASESVDNYGISFMNWFQQKPGKAPKLLIYAASNQGSG | 667 |
| | | VPSRFSGSGSGTDFTLTISSLQPDDFATYYCQQSKEVPWTFGCGTKVEIK | |
| 33-5 | VH1 | QVQLQQPGAEVKKPFASVKMSCKASGYTFTSYYIHWIKQTPGQGLEWVGVIYPGNDDISYN | 672 |
| | | QKFQGKATLTADKSSTTAYMQLSSLTSEDSAVYYCAREVRLRYFDVWGQGTTVTVSS | |
| | VH2 | QVQLVQSGAEVKKPGSSVKVSCKASGYTFTDYNMHWVRQAPGQCLEWIGYIYPYNGGTGYN | 666 |
| | | QKFKSKATITADESTNTAYMELSSLRSEDTAVYYCARGRPAMDYWGQGTLVTVSS | |
| | VL1 | EIVLTQSPGSLAVSPGERVTMSCKSSQSVFFSSSQKNYLAWYQQIPGQSPRLLIYWASTRE | 673 |
| | | SGVPDRFTGSGSGTDDTLTISSVQPEDLAIYYCHQYLSSRTFGQGTKLEIK | |
| | VL2 | DIQMTQSPSSLSASVGDRVTITCRASESVDNYGISFMNWFQQKPGKAPKLLIYAASNQGSG | 667 |
| | | VPSRFSGSGSGTDFTLTISSLQPDDFATYYCQQSKEVPWTFGCGTKVEIK | |
| 33-6 | VH1 | QVQLVQSGAEVKKPGSSVKVSCKASGYTFTDYNMHWVRQAPGQGLEWIGYIYPYNGGTGYN | 674 |
| | | QKFKSKATITADESTNTAYMELSSLRSEDTAVYYCARGRPAMDYWGQGTLVTVSS | 664 |
| | VH2 | EVQLVQSGAEVKKPGSSVKVSCKASGYTITDSNIHWVRQAPGQCLEWIGYIYPYNGGTDYN | |
| | | QKFKNRATLTVDNPTNTAYMELSSLRSEDTAFYYCVNGNPWLAYWGQGTLVTVSS | 675 |
| | VL1 | DIQMTQSPSSLSASVGDRVTITCRASESVDNYGISFMNWFQQKPGKAPKLLIYAASNQGSG | |
| | | VPSRFSGSGSGTDFTLTISSLQPDDFATYYCQQSKEVPWTFGQGTKVEIK | 665 |
| | VL2 | DIQLTQSPSTLSASVGDRVTITCRASESLDNYGIRFLTWFQQKPGKAPKLLMYAASNQGSG | |
| | | VPSRFSGSGSGTEFTLTISSLQPDDFATYYCQQTKEVPWSFGCGTKVEVK | |
| 33-7 | VH1 | QVQLVQSGAEVKKPGSSVKVSCKASGYTFTDYNMHWVRQAPGQGLEWIGYIYPYNGGTGYN | 674 |
| | | QKFKSKATITADESTNTAYMELSSLRSEDTAVYYCARGRPAMDYWGQGTLVTVSS | |
| | VH2 | QVQLQQPGAEVKKPGASVKMSCKASGYTFTSYYIHWIKQTPGQCLEWVGVIYPGNDDISYN | 668 |
| | | QKFQGKATLTADKSSTTAYMQLSSLTSEDSAVYYVAREVRLRYFDVWGQGTTVTVSS | |
| | VL1 | DIQMTQSPSSLSASVGDRVTITCRASESVDNYGISFMNWFQQKPGKAPKLLIYAASNQGSG | 675 |
| | | VPSRFSGSGSGTDFTLTISSLQPDDFATYYCQQSKEVPWTFGQGTKVEIK | |
| | VL2 | EIVLTQSPGSLAVSPGERVTMSCKSSQSVFFSSSQKNYLAWYQQIPGQSPRLLIYWASTRE | 669 |
| | | SGVPSRFTGSGSGTDFTLTISSVQPEDLAIYYCHQYLSSRTFGCGTKLEIK | |

TABLE 71

| Name | | Sequence | SEQ ID NO |
|---|---|---|---|
| 33-1 | VH | GAGGTTCAGTTGGTTCAGTCAGGAGCAGAGGTCAAAAAACCTGGAAGCTCTGTCAAAGTTA | 676 |
| | | GTTGTAAGGCCAGTGGATACACCATAACCGATTCAAATATACATTGGGTTAGGCAAGCACC | |
| | | AGGACAGTGCTTGGAATGGATCGGGTACATCTATCCATATAATGGGGGCACCGATTACAAC | |
| | | CAAAAGTTTAAGAATCGCGCCACACTCACTGTTGATAATCCAACCAATACAGCATACATGG | |
| | | AGTTGAGCAGTCTTCGGTCCGAGGACACTGCTTTTTACTATTGTGTGAACGGTAACCCATG | |
| | | GTTGGCCTATTGGGGCCAAGGTACACTTGTAACAGTTTCATCT | |
| | VL | GATATACAACTGACTCAGAGTCCCAGCACTCTCAGTGCAAGTGTAGGCGATAGAGTAACTA | 677 |
| | | TAACCTGTCGCGCCTCAGAATCTCTTGATAATTATGGGATCCGATTTCTTACTTGGTTTCA | |
| | | GCAAAAGCCTGGTAAAGCTCCTAAATTGCTCATGTATGCCGCCAGTAATCAGGGTTCAGGA | |
| | | GTTCCTAGTCGTTTCTCTGGGTCAGGAAGCGGCACAGAATTTACCCTTACAATTTCCAGCC | |
| | | TCCAGCCCGACGATTTCGCCACTTACTATTGCCAACAAACTAAAGAGGTTCCTTGGAGTTT | |
| | | TGGGTGTGGCACCAAGGTAGAAGTAAAA | |
| 33-2 | VH | CAGGTACAGCTTGTGCAATCTGGAGCTGAGGTCAAAAAACCAGGAAGTTCTGTAAAGGTGT | 678 |
| | | CTTGTAAGGCATCTGGTTACACATTTACCGATTACAACATGCATTGGGTACGTCAAGCCCC | |
| | | TGGCCAGTGCCTGGAATGGATCGGATATATATACCCCTACAACGGTGGAACAGGTTACAAC | |
| | | CAAAAATTCAAAAGCAAGGCTACTATCACTGCCGACGAGAGCACTAACACCGCTTACATGG | |
| | | AACTGTCTTCCTTGCGTTCAGAAGACACTGCCGTGTATTATTGTGCTCGGGGGCGACCCGC | |
| | | AATGGACTATTGGGGCCAAGGTACATTGGTGACTGTCAGTTCC | |
| | VL | GACATCCAAATGACACAGTCCCCTAGCTCACTTTCAGCAAGCGTGGGGGACCGAGTTACTA | 679 |
| | | TCACATGCCGCGCAAGTGAGTCTGTGGACAACTATGGAATATCATTCATGAACTGGTTCCA | |
| | | GCAGAAACCTGGGAAAGCACCCAAGCTGCTTATCTACGCAGCAAGTAATCAGGGTAGTGGC | |
| | | GTCCCTTCTCGATTCAGTGGGAGCGGTAGCGGCACCGACTTCACCCTTACCATCTCATCAC | |
| | | TTCAACCCGATGATTTTGCTACCTACTATTGCCAGCAATCCAAGGAAGTTCCTTGGACCTT | |
| | | CGGGTGTGGGACAAAGGTAGAGATTAAA | |
| 33-3 | VH | CAAGTCCAACTTCAGCAGCCTGGAGCTGAGGTAGTGAAACCCGGCGCATCTGTAAAAATGA | 680 |
| | | GCTGCAAAGCATCAGGTTACACATTTACATCCTACTACATCCATTGGATCAAGCAAACACC | |
| | | AGGCCAATGTCTTGAGTGGGTTGGCGTCATTTACCCAGGAAACGATGATATATCTTACAAT | |
| | | CAGAAATTTCAAGGGAAAGCCACACTTACAGCCGACAAGAGTTCCACAACTGCATATATGC | |
| | | AACTCTCCTCCCTGACATCTGAAGACAGTGCCGTATACTATTGTGCTCGTGAAGTCAGGCT | |
| | | CAGATACTTTGACGTGTGGGGTCAAGGCACAACCGTCACCGTCAGTAGC | |
| | VL | GAGATAGTCCTCACCCAATCACCAGGTAGTCTGGCTGTTAGCCCAGGTGAGCGAGTTACTA | 681 |
| | | TGTCTTGCAAGTCCTCTCAATCTGTGTTCTTTTCATCAAGTCAGAAGAATTATCTTGCCTG | |
| | | GTATCAGCAGATTCCTGGTCAATCTCCCCGACTGCTTATTTACTGGGCTTCAACTCGGGAG | |
| | | TCAGGGGTTCCCGACCGATTCACAGGGTCTGGGTCAGGAACTGACTTTACCCTTACTATCA | |
| | | GCTCTGTGCAGCCTGAAGACCTCGCAATATATTATTGTCATCAGTACCTCTCTTCTCGCAC | |
| | | TTTTGGATGTGGCACCAAATTGGAGATTAAG | |
| 33-4 | VH1 | GAGGTTCAGTTGGTTCAGTCAGGAGCAGAGGTCAAAAAACCTGGAAGCTCTGTCAAAGTTA | 682 |
| | | GTTGTAAGGCCAGTGGATACACCATAACCGATTCAAATATACATTGGGTTAGGCAAGCACC | |
| | | AGGACAGTCCTTGGAATGGATCGGGTACATCTATCCATATAATGGGGGCACCGATTACAAC | |
| | | CAAAAGTTTAAGAATCGCGCCACACTCACTGTTGATAATCCAACCAATACAGCATACATGG | |
| | | AGTTGAGCAGTCTTCGGTCCGAGGACACTGCTTTTTACTATTGTGTGAACGGTAACCCATG | |
| | | GTTGGCCTATTGGGGCCAAGGTACACTTGTAACAGTTTCATCC | |
| | VH2 | CAGGTACAGCTTGTGCAATCTGGAGCTGAGGTCAAAAAACCAGGAAGTTCTGTAAAGGTGT | 678 |
| | | CTTGTAAGGCATCTGGTTACACATTTACCGATTACAACATGCATTGGGTACGTCAAGCCCC | |
| | | TGGCCAGTGCCTGGAATGGATCGGATATATATACCCCTACAACGGTGGAACAGGTTACAAC | |
| | | CAAAAATTCAAAAGCAAGGCTACTATCACTGCCGACGAGAGCACTAACACCGCTTACATGG | |
| | | AACTGTCTTCCTTGCGTTCAGAAGACACTGCCGTGTATTATTGTGCTCGGGGGCGACCCGC | |
| | | AATGGACTATTGGGGCCAAGGTACATTGGTGACTGTCAGTTCC | |
| | VL1 | GATATACAACTGACTCAGAGTCCCAGCACTCTCAGTGCAAGTGTAGGCGATAGAGTAACTA | 683 |
| | | TAACCTGTCGCGCCTCAGAATCTCTTGATAATTATGGGATCCGATTTCTTACTTGGTTTCA | |
| | | GCAAAAGCCTGGTAAAGCTCCTAAATTGCTCATGTATGCCGCCAGTAATCAGGGTTCAGGA | |
| | | GTTCCTAGTCGTTTCTCTGGGTCAGGAAGCGGCACAGAATTTACCCTTACAATTTCCAGCC | |
| | | TCCAGCCCGACGATTTCGCCACTTACTATTGCCAACAAACTAAAGAGGTTCCTTGGAGTTT | |
| | | TGGGCAAGGCACCAAGGTAGAAGTAAAA | |
| | VL2 | GACATCCAAATGACACAGTCCCCTAGCTCACTTTCAGCAAGCGTGGGGGACCGAGTTACTA | 679 |
| | | TCACATGCCGCGCAAGTGAGTCTGTGGACAACTATGGAATATCATTCATGAACTGGTTCCA | |
| | | GCAGAAACCTGGGAAAGCACCCAAGCTGCTTATCTACGCAGCAAGTAATCAGGGTAGTGGC | |
| | | GTCCCTTCTCGATTCAGTGGGAGCGGTAGCGGCACCGACTTCACCCTTACCATCTCATCAC | |
| | | TTCAACCCGATGATTTTGCTACCTACTATTGCCAGCAATCCAAGGAAGTTCCTTGGACCTT | |
| | | CGGGTGTGGGACAAAGGTAGAGATTAAA | |
| 33-5 | VH1 | CAAGTCCAACTTCAGCAGCCTGGAGCTGAGGTAGTGAAACCCGGCGCATCTGTAAAAATGA | 684 |
| | | GCTGCAAAGCATCAGGTTACACATTTACATCCTACTACATCCATTGGATCAAGCAAACACC | |
| | | AGGCCAAGGTCTTGAGTGGGTTGGCGTCATTTACCCAGGAAACGATGATATATCTTACAAT | |
| | | CAGAAATTTCAAGGGAAAGCCACACTTACAGCCGACAAGAGTTCCACAACTGCATATATGC | |
| | | AACTCTCCTCCCTGACATCTGAAGACAGTGCCGTATACTATTGTGCTCGTGAAGTCAGGCT | |
| | | CAGATACTTTGACGTGTGGGGTCAAGGCACAACCGTCACCGTCAGTAGC | |
| | VH2 | CAGGTACAGCTTGTGCAATCTGGAGCTGAGGTCAAAAAACCAGGAAGTTCTGTAAAGGTGT | 678 |
| | | CTTGTAAGGCATCTGGTTACACATTTACCGATTACAACATGCATTGGGTACGTCAAGCCCC | |
| | | TGGCCAGTGCCTGGAATGGATCGGATATATATACCCCTACAACGGTGGAACAGGTTACAAC | |
| | | CAAAAATTCAAAAGCAAGGCTACTATCACTGCCGACGAGAGCACTAACACCGCTTACATGG | |
| | | AACTGTCTTCCTTGCGTTCAGAAGACACTGCCGTGTATTATTGTGCTCGGGGGCGACCCGC | |
| | | AATGGACTATTGGGGCCAAGGTACATTGGTGACTGTCAGTTCC | |
| | VL1 | GAGATAGTCCTCACCCAATCACCAGGTAGTCTGGCTGTTAGCCCAGGTGAGCGAGTTACTA | 685 |
| | | TGTCTTGCAAGTCCTCTCAATCTGTGTTCTTTTCATCAAGTCAGAAGAATTATCTTGCCTG | |
| | | GTATCAGCAGATTCCTGGTCAATCTCCCCGACTGCTTATTTACTGGGCTTCAACTCGGGAG | |
| | | TCAGGGGTTCCCGACCGATTCACAGGGTCTGGGTCAGGAACTGACTTTACCCTTACTATCA | |
| | | GCTCTGTGCAGCCTGAAGACCTCGCAATATATTATTGTCATCAGTACCTCTCTTCTCGCAC | |
| | | TTTTGGACAGGGCACCAAATTGGAGATTAAG | |
| | VL2 | GACATCCAAATGACACAGTCCCCTAGCTCACTTTCAGCAAGCGTGGGGGACCGAGTTACTA | 679 |
| | | TCACATGCCGCGCAAGTGAGTCTGTGGACAACTATGGAATATCATTCATGAACTGGTTCCA | |
| | | GCAGAAACCTGGGAAAGCACCCAAGCTGCTTATCTACGCAGCAAGTAATCAGGGTAGTGGC | |
| | | GTCCCTTCTCGATTCAGTGGGAGCGGTAGCGGCACCGACTTCACCCTTACCATCTCATCAC | |
| | | TTCAACCCGATGATTTTGCTACCTACTATTGCCAGCAATCCAAGGAAGTTCCTTGGACCTT | |
| | | CGGGTGTGGGACAAAGGTAGAGATTAAA | |
| 33-6 | VH1 | CAGGTACAGCTTGTGCAATCTGGAGCTGAGGTCAAAAAACCAGGAAGTTCTGTAAAGGTGT | 686 |
| | | CTTGTAAGGCATCTGGTTACACATTTACCGATTACAACATGCATTGGGTACGTCAAGCCCC | |
| | | TGGCCAGGGCCTGGAATGGATCGGATATATATACCCCTACAACGGTGGAACAGGTTACAAC | |
| | | CAAAAATTCAAAAGCAAGGCTACTATCACTGCCGACGAGAGCACTAACACCGCTTACATGG | |
| | | AACTGTCTTCCTTGCGTTCAGAAGACACTGCCGTGTATTATTGTGCTCGGGGGCGACCCGC | |
| | | AATGGACTATTGGGGCCAAGGTACATTGGTGACTGTCAGTTCC | |
| | VH2 | GAGGTTCAGTTGGTTCAGTCAGGAGCAGAGGTCAAAAAACCTGGAAGCTCTGTCAAAGTTA | 676 |
| | | GTTGTAAGGCCAGTGGATACACCATAACCGATTCAAATATACATTGGGTTAGGCAAGCACC | |
| | | AGGACAGTGCTTGGAATGGATCGGGTACATCTATCCATATAATGGGGGCACCGATTACAAC | |
| | | CAAAAGTTTAAGAATCGCGCCACACTCACTGTTGATAATCCAACCAATACAGCATACATGG | |
| | | AGTTGAGCAGTCTTCGGTCCGAGGACACTGCTTTTTACTATTGTGTGAACGGTAACCCATG | |
| | | GTTGGCCTATTGGGGCCAAGGTACACTTGTAACAGTTTCATCT | |
| | VL1 | GACATCCAAATGACACAGTCCCCTAGCTCACTTTCAGCAAGCGTGGGGGACCGAGTTACTA | 687 |
| | | TCACATGCCGCGCAAGTGAGTCTGTGGACAACTATGGAATATCATTCATGAACTGGTTCCA | |
| | | GCAGAAACCTGGGAAAGCACCCAAGCTGCTTATCTACGCAGCAAGTAATCAGGGTAGTGGC | |
| | | GTCCCTTCTCGATTCAGTGGGAGCGGTAGCGGCACCGACTTCACCCTTACCATCTCATCAC | |
| | | TTCAACCCGATGATTTTGCTACCTACTATTGCCAGCAATCCAAGGAAGTTCCTTGGACCTT | |
| | | CGGGCAAGGGACAAAGGTAGAGATTAAA | |
| | VL2 | GATATACAACTGACTCAGAGTCCCAGCACTCTCAGTGCAAGTGTAGGCGATAGAGTAACTA | 677 |
| | | TAACCTGTCGCGCCTCAGAATCTCTTGATAATTATGGGATCCGATTTCTTACTTGGTTTCA | |
| | | GCAAAAGCCTGGTAAAGCTCCTAAATTGCTCATGTATGCCGCCAGTAATCAGGGTTCAGGA | |
| | | GTTCCTAGTCGTTTCTCTGGGTCAGGAAGCGGCACAGAATTTACCCTTACAATTTCCAGCC | |
| | | TCCAGCCCGACGATTTCGCCACTTACTATTGCCAACAAACTAAAGAGGTTCCTTGGAGTTT | |
| | | TGGGTGTGGCACCAAGGTAGAAGTAAAA | |
| 33-7 | VH1 | CAGGTACAGCTTGTGCAATCTGGAGCTGAGGTCAAAAAACCAGGAAGTTCTGTAAAGGTGT | 686 |
| | | CTTGTAAGGCATCTGGTTACACATTTACCGATTACAACATGCATTGGGTACGTCAAGCCCC | |
| | | TGGCCAGGGCCTGGAATGGATCGGATATATATACCCCTACAACGGTGGAACAGGTTACAAC | |
| | | CAAAAATTCAAAAGCAAGGCTACTATCACTGCCGACGAGAGCACTAACACCGCTTACATGG | |
| | | AACTGTCTTCCTTGCGTTCAGAAGACACTGCCGTGTATTATTGTGCTCGGGGGCGACCCGC | |
| | | AATGGACTATTGGGGCCAAGGTACATTGGTGACTGTCAGTTCC | |
| | VH2 | CAAGTCCAACTTCAGCAGCCTGGAGCTGAGGTAGTGAAACCCGGCGCATCTGTAAAAATGA | 680 |
| | | GCTGCAAAGCATCAGGTTACACATTTACATCCTACTACATCCATTGGATCAAGCAAACACC | |
| | | AGGCCAATGTCTTGAGTGGGTTGGCGTCATTTACCCAGGAAACGATGATATATCTTACAAT | |
| | | CAGAAATTTCAAGGGAAAGCCACACTTACAGCCGACAAGAGTTCCACAACTGCATATATGC | |
| | | AACTCTCCTCCCTGACATCTGAAGACAGTGCCGTATACTATTGTGCTCGTGAAGTCAGGCT | |
| | | CAGATACTTTGACGTGTGGGGTCAAGGCACAACCGTCACCGTCAGTAGC | |
| | VL1 | GACATCCAAATGACACAGTCCCCTAGCTCACTTTCAGCAAGCGTGGGGGACCGAGTTACTA | 687 |
| | | TCACATGCCGCGCAAGTGAGTCTGTGGACAACTATGGAATATCATTCATGAACTGGTTCCA | |
| | | GCAGAAACCTGGGAAAGCACCCAAGCTGCTTATCTACGCAGCAAGTAATCAGGGTAGTGGC | |
| | | GTCCCTTCTCGATTCAGTGGGAGCGGTAGCGGCACCGACTTCACCCTTACCATCTCATCAC | |
| | | TTCAACCCGATGATTTTGCTACCTACTATTGCCAGCAATCCAAGGAAGTTCCTTGGACCTT | |
| | | CGGGCAAGGGACAAAGGTAGAGATTAAA | |
| | VL2 | GAGATAGTCCTCACCCAATCACCAGGTAGTCTGGCTGTTAGCCCAGGTGAGCGAGTTACTA | 681 |
| | | TGTCTTGCAAGTCCTCTCAATCTGTGTTCTTTTCATCAAGTCAGAAGAATTATCTTGCCTG | |
| | | GTATCAGCAGATTCCTGGTCAATCTCCCCGACTGCTTATTTACTGGGCTTCAACTCGGGAG | |
| | | TCAGGGGTTCCCGACCGATTCACAGGGTCTGGGTCAGGAACTGACTTTACCCTTACTATCA | |
| | | GCTCTGTGCAGCCTGAAGACCTCGCAATATATTATTGTCATCAGTACCTCTCTTCTCGCAC | |
| | | TTTTGGATGTGGCACCAAATTGGAGATTAAG | |

Table 72 below shows the heavy chain and light chain CDR sequences of the engineered antibodies targeting CD33.

TABLE 72

| Name | CDR | Sequence | SEQ ID NO |
|---|---|---|---|
| 33-1 | CDR-H1 | DSNIH | 688 |
| | CDR-H2 | YIYTYNGGTDYNQKFKN | 689 |
| | CDR-H3 | GNPWLAY | 690 |
| | CDR-L1 | RASESLDNYGIRFLT | 691 |
| | CDR-L2 | AASNQGS | 692 |
| | CDR-L3 | QQTKEVTWS | 693 |
| 33-2 | CDR-H1 | DYNMH | 694 |
| | CDR-H2 | YIYPYNGGTGYNQKFKS | 695 |
| | CDR-H3 | GRPAMDY | 696 |
| | CDR-L1 | RASESVDNYGISFMN | 697 |
| | CDR-L2 | AASNQGS | 698 |
| | CDR-L3 | QQSKEVPWT | 699 |
| 33-3 | CDR-H1 | SYYIH | 700 |
| | CDR-H2 | VIYPGNDDISYNQKFQG | 701 |
| | CDR-H3 | EVRLRYFDV | 702 |
| | CDR-L1 | KSSQSVFFSSSQKNYLA | 703 |
| | CDR-L3 | WASTRES | 704 |
| | CDR-L3 | HQYLSSRT | 705 |
| 33-4 | V1 CDR-H1 | DSNIH | 688 |
| | V1 CDR-H2 | YIYPYNGGTDYNQKFKN | 689 |
| | V1 CDR-H3 | GNPWLAY | 690 |
| | V1 CDR-L1 | RASESLDNYGIRFLT | 691 |
| | V1 CDR-L2 | AASNQGS | 692 |
| | V1 CDR-L3 | QQTKEVPWS | 693 |
| | V2 CDR-H1 | DYNMH | 694 |
| | V2 CDR-H2 | YIYPYNGGTGYNQKFKS | 695 |
| | V2 CDR-H3 | GRPAMDY | 696 |
| | V2 CDR-L1 | RASESVDNYGISFMN | 697 |
| | V2 CDR-L2 | AASNQGS | 698 |
| | V2 CDR-L3 | QQSKEVPWT | 699 |
| 33-5 | V1 CDR-H1 | SYYIH | 700 |
| | V1 CDR-H2 | VIYTGNDDISYNQKFQG | 701 |
| | V1 CDR-H3 | EVRLRYFDV | 702 |
| | V1 CDR-L1 | KSSQSVFFSSSQKNYLA | 703 |
| | V1 CDR-L2 | WASTRES | 704 |
| | V1 CDR-L3 | HQYLSSRT | 705 |
| | V2 CDR-H1 | DYNMH | 694 |
| | V2 CDR-H2 | YIYPYNGGTGYNQKFKS | 695 |
| | V2 CDR-H3 | GRPAMDY | 696 |
| | V2 CDR-L1 | RASESVDNYGISFMN | 697 |
| | V2 CDR-L2 | AASNQGS | 698 |
| | V2 CDR-L3 | QQSKEVPWT | 699 |
| 33-6 | V1 CDR-H1 | DYNMH | 694 |
| | V1 CDR-H2 | YIYPYNGGTGYNQKFKS | 695 |
| | V1 CDR-H3 | GRPAMDY | 696 |
| | V1 CDR-L1 | RASESVDNYGISFMN | 697 |
| | V1 CDR-L2 | AASNQGS | 698 |
| | V1 CDR-L3 | QQSKEVPWT | 699 |
| | V2 CDR-H1 | DSNIH | 688 |
| | V2 CDR-H2 | YIYPYNGGTDYNQKFKN | 689 |
| | V2 CDR-H3 | GNPWLAY | 690 |
| | V2 CDR-L1 | RASESLDNYGIRFLT | 691 |
| | V2 CDR-L2 | AASNQGS | 692 |
| | V2 CDR-L3 | QQTKEVPWS | 693 |
| 33-7 | V1 CDR-H1 | DYNMH | 694 |
| | V1 CDR-H2 | YIYPYNGGTGYNQKFKS | 695 |
| | V1 CDR-H3 | GRPAMDY | 696 |
| | V1 CDR-L1 | RASESVDNYGISFMN | 697 |
| | V1 CDR-L2 | AASNQGS | 698 |
| | V1 CDR-L3 | QQSKEVPWT | 699 |
| | V2 CDR-H1 | DYNMH | 694 |
| | V2 CDR-H2 | YIYPYNGGTGYNQKFKS | 695 |
| | V2 CDR-H3 | GRPAMDY | 696 |
| | V2 CDR-L1 | RASESVDNYGISFMN | 697 |
| | V2 CDR-L2 | AASNQGS | 698 |
| | V2 CDR-L3 | QQSKEVPWT | 699 |

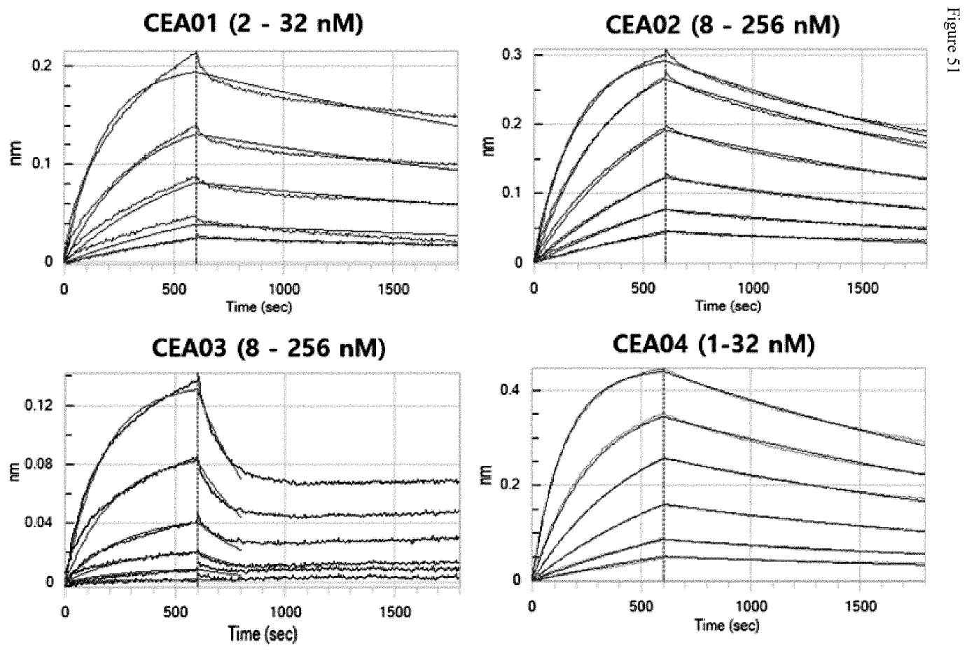

The CD33 protein binding constants of 33-1, 33-2, 33-3, 33-4, 33-5, 33-6, and 33-7 were determined using Octet Red96e (Sartorius). In order to analyze the binding constants of the seven antibodies, the human CD33 recombinant protein (Sino Biologicals, 12238-H08H) was loaded onto the Anti-Penta-HIS (HIS1K) biosensor (Sartorius, 18-5120). Then, the seven antibodies were added in a binding reaction (600 seconds) and a dissociation reaction (1,200 seconds) at various concentrations, and their affinities for CD33 were calculated (FIG. 50, Table 73). Table 73 below illustrates the binding constants of the engineered antibodies targeting CD33.

TABLE 73

| Antibodies | Antigen | Binding mode | K_{D} (nM) | Kₐ (1/Ms) | K_{d} (1/s) |
|---|---|---|---|---|---|
| 33-1 | rhCD33 | Monovalent | 1.7835 | 2.80E+05 | 4.99E−04 |
| 33-2 | rhCD33 | Monovalent | 5.5540 | 8.01E+05 | 4.45E−03 |
| 33-3 | rhCD33 | Monovalent | 0.5163 | 2.24E+05 | 1.16E−04 |
| 33-4 | rhCD33 | Biparatopic | 0.0851 | 2.79E+05 | 8.11E+02 |
| 33-5 | rhCD33 | Biparatopic | 0.0641 | 2.01E+05 | 4.07E+02 |
| 33-6 | rhCD33 | Biparatopic | 0.0772 | 3.60E+05 | 1.08E+03 |
| 33-7 | rhCD33 | Biparatopic | 0.1290 | 1.37E+05 | 1.24E+02 |

### Example 22. Design, Preparation and Analysis of Antibody Structure Targeting CEACAM5

The light chain and heavy chain variant polypeptide sequences of the antibodies that specifically bind to the CEACAM5 protein are shown in Table 74. For CEA01, expression vectors consisting of the sequences corresponding to Fc-Hole (SEQ ID NO: 7), CEA01 HC (SEQ ID NO: 590), and CEA01 LC (SEQ ID NO: 591) were co-transfected into EXPICHO-S™ (Gibco, A29127) (FIG. 41a, Table 74), and purification and analysis were performed in the same manner as described in Example 1. Expression, purification and analysis were performed for CEA02, CEA03, and CEA04 in the same manner as mentioned above (Table 74).

TABLE 74

| | | SEQ |
|---|---|---|
| Name | Sequence | ID NO |
| CEA01 HC | EVQLVESGGGVVQPGRSLRLSCSASGFDFTTYWMSWVRQAPGKCLEWIGEIHPDSSTIN | 706 |
| | YAPSLKDRFTISRDNAKNTLFLQMDSLRPEDTGVYFCASLYFGFPWFAYWGQGTPVTVS | |
| | SASTKGPSVFPLAPSSKSTSGGTAALGCLVKDYFPEPVTVSWNSGALTSGVHTFPAVLQ | |
| | SSGLYSLSSVVYVYSSSLGTQTYICNVNHKPSNTKVDKKVEPKSCDKTHTCPPCPAPEL | |
| | LGGPSVFLFPPKPKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAKGQPREPQVYTLP | |
| | PCREEMTKNQVSLWCLVKGFYPSDIAVEWESNGQPENNYKTTPPVLDSDGSFFLYSKLY | |
| | VDKSRWQQGNVFSCSVMHEALHNHYTQKSLSLSPGK | |
| CEA01 LC | DIQLTQSPSSLSASVGDRVTITCKASQDVGTSVAWYQQKPGKAPKLLIYWTSTRHTGVT | 707 |
| | SRFSGSGSGTDFTFTISSLQPEDIATYYCQQYSLYRSFGCGTKVEIKRTVAAPSVFIFP | |
| | PSDEQLKSGTASVVCLLNNFYPREAKVQWKVDNALQSGNSQESVTEQDSKDSTYSLSST | |
| | LTLSKADYEKHKVYACEVTHQGLSSPVTKSFNRGECGGGGSGGGGSGGGGSEPKSSDKT | |
| | HTCPPCPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTISKAKG | |
| | QPREPQVYTLPPCREEMTKNQVSLWCLVKGFYPSDIAVEWESNGQPENNYKTTPPVLDS | |
| | DGSFFLYSKLTVDKSRWQQGNVFSCSVMHEALHNHYTQKSLSLSPGK | |
| CEA02 HC | EVQLVESGGGLVQPGRSLRLSCAASGFTVSSYWMHWVRQAPGKCLEWVGFILNKANGGT | 708 |
| | TEYAASVKGRFTISRDDSKNTLYLQMNSLRAEDTAVYYCARDRGLRFYFDYWGWGTTVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAKGQPREPQVYT | |
| | LPPCREEMTKNQVSLWCLVKGFYPSDIAVEWESNGQPENNYKTTPPVLDSDGSFFLYSK | |
| | LTVDKSRWQQGNVFSCSVMHEALHNHYTQKSLSLSPGK | |
| CEA02 LC | QAVLTQPASLSASPGASASLTCTLRRGINVGAYSIYWYQQKPGSPPQYLLRYKSDSDKQ | 709 |
| | QGAGVSSRFSASKDASANAGILLISGLQSEDEADYYCMIWHSGASAVFGCGTKLTVLGQ | |
| | PKAAPSVTLFPPSSEELQANKATLVCLISDFYPGAVTVAWKADSSPVKAGVETTTPSKQ | |
| | SNNKYAASSYLSLTPEQWKSHRSYSCQVTHEGSTVEKTVAPTECSGGGGSGGGGSGGGG | |
| | SEPKSSDKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKALPAPI | |
| | EKTISKAKGQPREPQVYTLPPCREEMTKNQVSLWCLVKGFYPSDIAVEWESNGQPENNY | |
| | KTTPPVLDSDGSFFLYSKLTVDKSRWQQGNVFSCSVMHEALHNHYTQKSLSLSPGK | |
| CEA03 HC | EVQLQQSGPGLVRPSQTLSLTCTASGFNIKDNYMHWVRQPPGRCLEWIGWIDPENGDTE | 710 |
| | YAPKFRGRVTMLADTSKNQFSLRLSSVTAADTAVYYCHVLIYAGYLAMDYWGQGTLVTV | |
| | SSASTKGPSVFPLAPSSKSTSGGTAALGCLVKDYFPEPVTVSWNSGALTSGVHTFPAVL | |
| | QSSGLYSLSSVVTVPSSSLGTQTYICNVNHKPSNTKVDKKVEPKSCDKTHTCPPCPAPE | |
| | LLGGPSVFLFPPKPKDTLMISRTPEVTCVVVDVSHEDPEVKFNWYVDGVEVHNAKTKPR | |
| | EEQYNSTYRVVSVLTVLHQDWLNGKEYKCKVSNKALPAPIEKTISKAKGQPREPQVYTL | |
| | PPCREEMTKNQVSLWCLVKGFYPSDIAVEWESNGQPENNYKTTPPVLDSDGSFFLYSKL | |
| | TVDKSRWQQGNVFSCVMHEALHNHYTQKSLSLSPGK | |
| CEA03 LC | DIQMTQSPSSLSASVGDRVTITCSASSSVTYMHWYQQKPGKAPKLWIYSTSNLASGVPS | 711 |
| | RFSGSGSGTDYTFTISSLQPEDIATYYCQQRSTYPLTFGCGTKLEIKRTVAAPSVFIFP | |
| | PSDEQLKSGTASVVCLLNNFYPREAKVQWKVDNALQSGNSQESVTEQDSKDSTYSLSST | |
| | LTLSKADYEKHKVYACEVTHQGLSSPVTKSFNRGECGGGGSGGGGSGGGGSEPKSSDKT | |
| | HTCPPCPAPELLGGPSVFLFPPKPKDTLMISRTPEVTCVVVDVSHEDPEVKFNWYVDGV | |
| | EVHNAKTKPREEQYNSTYRVVSVLTVLHQDWLNGKEYKCKVSNKALPAPIEKTISKAKG | |
| | QPREPQVYTLPPCREEMTKNQVSLWCLVKGFYPSDIAVEWESNGQPENNYKTTPPVLDS | |
| | DGSFFLYSKLTVDKSRWQQGNVFSCSVMHEALHNHTQKSLSLSPGK | |
| CEA04 HC | EVQLVESGGGLVQPGGSLRLSCAASGFNIKDTYMHWVRQAPGKCLEWVARIDPANGNSK | 712 |
| | YADSVKGRFTISADTSKNTAYLQMNSLRAEDTAVYYCAPFGYYVSDYAMAYWGQGTLVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAKGQPREPQVYT | |
| | LPPCREEMTKNQVSLWCLVKGFYPSDIAVEWESNGQPENNYKTTPPVLDSDGSFFLYSK | |
| | LTVDKSRWQQGNVFSCSVMHEALHNHYTQKSLSLSPGK | |
| CEA04 LC | DIQLTQSPSSLSASVGDRVTITCRAGESVDIFGVGFLHWYQQKPGKAPKLLIYRASNLE | 713 |
| | SGVPSRFSGSGSRTDFTLTISSLQPEDFATYYCQQTNEDPYTFGCGTKVEIKRTVAAPS | |
| | VFIFPPSDEQLKSGTASVVCLLNNFYPREAKVQWKVDNALQSGNSQESVTEQDSKDSTY | |
| | SLSSTLTLSKADYEKHKVYACEVTHQGLSSPVTKSFNRGECGGGGSGGGGSGGGGSEPK | |
| | SSDKTHTCPPCPAPELLGGPSVFLFPPKPKDTLMISRTPEVTCVVVDVSHEDPEVKFNW | |
| | YVDGVEVHNAKTKPREEQYNSTYRVVSVLTVLHQDWLNGKEYKCKVSNKALPAPIEKTI | |
| | SKAKGQPREPQVYTLPPCREEMTKNQVSLWCLVKGFYPSDIAVEWESNGQPENNYKTTP | |
| | PVLDSDGSFFLYSKLTVDKSRWQQGNVFSCSVMHEALHNHYTQKSLSLSPGK | |

Table 75 below shows the heavy chain and light chain nucleotide sequences of the engineered antibodies targeting CEACAM5.

TABLE 75

| | | SEQ |
|---|---|---|
| Name | Sequence | ID NO |
| CEA01 HC | GAAGTGCAACTCGTAGAATCCGGCGGGGGCGTCGTACAGCCCGGCCGTTCTTTGAGACT | 714 |
| | TAGCTGTAGTGCTTCTGGGTTTGACTTCACTACATACTGGATGTCATGGGTAAGACAGG | |
| | CACCTGGCAAGTGCCTTGAATGGATCGGTGAAATCCATCCCGACAGCTCCACAATCAAC | |
| | TACGCCCCAAGTTTGAAAGACCGGTTCACCATATCTCGTGACAACGCCAAGAATACATT | |
| | GTTCCTTCAGATGGATAGTCTTCGTCCAGAGGATACTGGGGTATATTTTTGTGCCAGCT | |
| | TGTATTTTGGCTTCCCCTGGTTCGCTTATTGGGGCCAAGGTACACCCGTCACTGTCTCT | |
| | TCTGCTAGCACCAAAGGACCTAGTGTTTTTGGTGTTGCCCCTTCCTCAAAGTCTACCTC | |
| | TGGGGGGACAGCCGCTCTGGGCTGCCTGGTCAAGGATTATTTCCCAGAGCCTGTCACTG | |
| | TCAGTTGGAACTCTGGAGCCTTGACTTCTGGTGTTCATACATTTCCTGCTGTCCTTCAA | |
| | AGCAGCGGCTTGTACTCATTGTCTTCTGTTGTGACAGTACCCTCAAGCAGCCTCGGCAC | |
| | TCAGACATACATCTGCAATGTCAACCACAAACCCTCAAATACAAAGGTAGATAAAAAAG | |
| | TCGAACCAAAGTCTTGTGACAAAACTCACACGTGCCCACCGTGCCCAGCACCTGAACTC | |
| | CTGGGGGGACCGTCAGTCTTCCTCTTCCCCCCAAAACCCAAGGACACCCTCATGATCTC | |
| | CCGGACCCCTGAGGTCACATGCGTGGTGGTGGACGTGAGCCACGAAGACCCTGAGGTCA | |
| | AGTTCAACTGGTACGTGGACGGCGTGGAGGTGCATAATGCCAAGACAAAGCCGCGGGAG | |
| | GAGCAGTACAACAGCACGTACCGTGTGGTCAGCGTCCTCACCGTCCTGCACCAGGACTG | |
| | GCTGAATGGCAAGGAGTACAAGTGCAAGGTCTCCAACAAAGCCCTCCCAGCCCCCATCG | |
| | AGAAAACCATCTCCAAAGCCAAAGGGCAGCCCCGAGAACCACAGGTGTACACCCTGCCC | |
| | CCATGCCGGGAGGAGATGACCAAGAACCAGGTCAGCCTGTGGTGCCTGGTCAAAGGCTT | |
| | CTATCCCAGCGACATCGCCGTGGAGTGGGAGAGCAATGGGCAGCCGGAGAACAACTACA | |
| | AGACCACGCCTCCCGTGCTGGACTCCGACGGCTCCTTCTTCCTCTACAGCAAGCTCACC | |
| | GTGGACAAGAGCAGGTGGCAGCAGGGGAACGTCTTCTCATGCTCCGTGATGCATGAGGC | |
| | TCTGCACAACCACTACACGCAGAAGAGCCTCTCCCTGTCTCCGGGTAAA | |
| CEA01 LC | GACATCCAACTTACCCAGTCACCCTCATCTCTTAGTGCCTCTGTAGGGGACCGAGTTAC | 715 |
| | TATTACATGTAAAGCCAGTCAAGATGTTGGCACCTCAGTAGCATGGTATCAACAAAAGC | |
| | CTGGTAAAGCCCCAAAACTGCTGATCTATTGGACAAGCACACGACATACAGGAGTGCCA | |
| | AGTCGCTTCAGCGGTTCAGGTTCAGGCACAGATTTTACATTCACTATATCAAGCCTGCA | |
| | ACCCGAGGACATTGCCACATATTACTGCCAGCAATATAGTCTGTATCGTAGCTTCGGAT | |
| | GTGGCACCAAGGTTGAAATTAAGCGTACGGTGGCAGCTCCCAGCGTTTTTATCTTTCCC | |
| | CCATCCGACGAGCAGCTCAAGAGTGGCACTGCCTCTGTAGTTTGTTTGCTGAATAACTT | |
| | CTATCCACGTGAAGCAAAAGTACAGTGGAAGGTCGATAATGCCCTTCAGAGCGGTAACA | |
| | GCCAAGAAAGTGTTACCGAGCAAGATTCCAAAGATTCCACTTACAGTCTGTCCAGCACA | |
| | TTGACACTGAGTAAGGCTGATTACGAAAAACACAAGGTGTACGCATGCGAGGTGACACA | |
| | CCAAGGTCTTTCATCTCCTGTAACTAAGAGCTTTAACCGGGGAGAATGTGGTGGTGGGG | |
| | GCAGCGGGGGCGGAGGTAGTGGAGGCGGCGGTAGTGAACCAAAGAGTAGTGACAAAACT | |
| | CACACGTGCCCACCGTGCCCAGCACCTGAACTCCTGGGGGGACCGTCAGTCTTCCTCTT | |
| | CCCCCCAAAACCCAAGGACACCCTCATGATCTCCCGGACCCCTGAGGTCACATGCGTGG | |
| | TGGTGGACGTGAGCCACGAAGACCCTGAGGTCAAGTTCAACTGGTACGTGGACGGCGTG | |
| | GAGGTGCATAATGCCAAGACAAAGCCGCGGGAGGAGCAGTACAACAGCACGTACCGTGT | |
| | GGTCAGCGTCCTCACCGTCCTGCACCAGGACTGGCTGAATGGCAAGGAGTACAAGTGCA | |
| | AGGTCTCCAACAAAGCCCTCCCAGCCCCCATCGAGAAAACCATCTCCAAAGCCAAAGGG | |
| | CAGCCCCGAGAACCACAGGTGTACACCCTGCCCCCATGCCGGGAGGAGATGACCAAGAA | |
| | CCAGGTCAGCCTGTGGTGCCTGGTCAAAGGCTTCTATCCCAGCGACATCGCCGTGGAGT | |
| | GGGAGAGCAATGGGCAGCCGGAGAACAACTACAAGACCACGCCTCCCGTGCTGGACTCC | |
| | GACGGCTCCTTCTTCCTCTACAGCAAGCTCACCGTGGACAAGAGCAGGTGGCAGCAGGG | |
| | GAACGTCTTCTCATGCTCCGTGATGCATGAGGCTCTGCACAACCACTACACGCAGAAGA | |
| | GCCTCTCCCTGTCTCCGGGTAAA | |
| CEA02 HC | GAAGTTCAACTGGTGGAGTCTGGCGGCGGACTTGTCCAGCCAGGGCGAAGCCTGCGTCT | 716 |
| | CTCATGCGCTGCCTCCGGTTTCACTGTTTCTTCATACTGGATGCACTGGGTAAGACAGG | |
| | CTCCTGGCAAGTGTCTTGAATGGGTGGGCTTCATTTTGAACAAGGCAAACGGCGGGACT | |
| | ACCGAATACGCTGCCAGCGTTAAGGGTCGATTCACCATCTCAAGGGATGATTCTAAAAA | |
| | CACATTGTACCTTCAGATGAACTCTCTGAGGGCCGAGGACACAGCAGTCTACTATTGCG | |
| | CTAGAGATAGGGGGCTTAGATTCTATTTTGACTACTGGGGGCAGGGAACAACTGTCACC | |
| | GTTTCCAGTGCTAGCACCAAAGGACCTAGTGTTTTTCCTCTTGCCCCTTCCTCAAAGTC | |
| | TACCTCTGGGGGGACAGCCGCTCTGGGCTGCCTGGTCAAGGATTATTTCCCAGAGCCTG | |
| | TCACTGTCAGTTGGAACTCTGGAGCCTTGACTTCTGGTGTTCATACATTTCCTGCTGTC | |
| | CTTCAAAGCAGCGGCTTGTACTCATTGTCTTCTGTTGTGACAGTACCCTCAAGCAGCCT | |
| | CGGCACTCAGACATACATCTGCAATGTCAACCACAAACCCTCAAATACAAAGGTAGATA | |
| | AAAAAGTCGAACCAAAGTCTTGTGACAAAACTCACACGTGCCCACCGTGCCCAGCACCT | |
| | GAACTCCTGGGGGGACCGTCAGTCTTCCTCTTCCCCCCAAAACCCAAGGACACCCTCAT | |
| | GATCTCCCGGACCCCTGAGGTCACATGCGTGGTGGTGGACGTGAGCCACGAAGACCCTG | |
| | AGGTCAAGTTCAACTGGTACGTGGACGGCGTGGAGGTGCATAATGCCAAGACAAAGCCG | |
| | CGGGAGGAGCAGTACAACAGCACGTACCGTGTGGTCAGCGTCCTCACCGTCCTGCACCA | |
| | GGACTGGCTGAATGGCAAGGAGTACAAGTGCAAGGTCTCCAACAAAGCCCTCCCAGCCC | |
| | CCATCGAGAAAACCATCTCCAAAGCCAAAGGGCAGCCCCGAGAACCACAGGTGTACACC | |
| | CTGCCCCCATGCCGGGAGGAGATGACCAAGAACCAGGTCAGCCTGTGGTGCCTGGTCAA | |
| | AGGCTTCTATCCCAGCGACATCGCCGTGGAGTGGGAGAGCAATGGGCAGCCGGAGAACA | |
| | ACTACAAGACCACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATGCTCCGTGATGCA | |
| | TGAGGCTCTGCACAACCACTACACGCAGAAGAGCCTCTCCCTGTCTCCGGGTAAA | |
| CEA02 LC | CAGGCAGTGCTTACTCAGCCTGCTTCACTCTCTGCCTCACCAGGAGCATCTGCAAGCCT | 717 |
| | CACCTGCACATTGCGTCGAGGTATAAACGTAGGCGCTTACTCAATTTACTGGTACCAGC | |
| | AGAAACCTGGGAGCCCACCTCAATACCTCCTCCGATACAAGAGCGATTCTGATAAGCAA | |
| | CAGGGCAGTGGTGTATCCAGCAGATTTTCCGCCAGCAAGGATGCAAGCGCTAATGCAGG | |
| | TATTCTTCTCATTTCAGGCTTGCAAAGCGAGGACGAAGCAGACTACTACTGCATGATTT | |
| | GGCACTCCGGTGCCTCCGCAGTTTTTGGCTGCGGAACAAAGCTTACAGTCCTTGGTCAG | |
| | CCCAAGGCTGCCCCCTCGGTCACTCTGTTCCCGCCCTCCTCTGAGGAGCTTCAAGCCAA | |
| | CAAGGCCACACTGGTGTGTCTCATAAGTGACTTCTACCCGGGAGCCGTGACAGTGGCCT | |
| | GGAAGGCAGATAGCAGCCCCGTCAAGGCGGGAGTGGAGACCACCACACCCTCCAAACAA | |
| | AGCAACAACAAGTACGCGGCCAGCAGCTATCTGAGCCTGACGCCTGAGCAGTGGAAGTC | |
| | CCACAGAAGCTACAGCTGCCAGGTCACGCATGAAGGGAGCACCGTGGAGAAGACAGTGG | |
| | CCCCTACAGAATGTTCAGGTGGTGGGGGCAGCGGGGGCGGAGGTAGTGGAGGCGGCGGT | |
| | AGTGAACCAAAGAGTAGTGACAAAACTCACACGTGCCCACCGTGCCCAGCACCTGAACT | |
| | CCTGGGGGGACCGTCAGTCTTCCTCTTCCCCCCAAAACCCAAGGACACCCTCATGATCT | |
| | CCCGGACCCCTGAGGTCACATGCGTGGTGGTGGACGTGAGCCACGAAGACCCTGAGGTC | |
| | AAGTTCAACTGGTACGTGGACGGCGTGGAGGTGCATAATGCCAAGACAAAGCCGCGGGA | |
| | GGAGCAGTACAACAGCACGTACCGTGTGGTCAGCGTCCTCACCGTCCTGCACCAGGACT | |
| | GGCTGAATGGCAAGGAGTACAAGTGCAAGGTCTCCAACAAAGCCCTCCCAGCCCCCATC | |
| | GAGAAAACCATCTCCAAAGCCAAAGGGCAGCCCCGAGAACCACAGGTGTACACCCTGCC | |
| | CCCATGCCGGGAGGAGATGACCAAGAACCAGGTCAGCCTGTGGTGCCTGGTCAAAGGCT | |
| | TCTATCCCAGCGACATCGCCGTGGAGTGGGAGAGCAATGGGCAGCCGGAGAACAACTAC | |
| | AAGACCACGCCTCCCGTGCTGGACTCCGACGGCTCCTTCTTCCTCTACAGCAAGCTCAC | |
| | CGTGGACAAGAGCAGGTGGCAGCAGGGGAACGTCTTCTCATGCTCCGTGATGCATGAGG | |
| | CTCTGCACAACCACTACACGCAGAAGAGCCTCTCCCTGTCTCCGGGTAAA | |
| CEA03 HC | GAGGTACAACTGCAGCAAAGCGGTCCAGGCCTTGTTCGGCCTAGCCAGACTCTGTCACT | 718 |
| | GACCTGTACCGCAAGCGGTTTCAATATCAAGGACAACTATATGCACTGGGTCCGCCAGC | |
| | CTCCAGGACGCTGTTTGGAGTGGATAGGATGGATAGACCCAGAAAATGGGGATACCGAA | |
| | TACGCTCCAAAATTTCGTGGACGAGTTACCATGCTGGCCGATACATCCAAAAACCAGTT | |
| | TTCTCTGAGGCTCTCCAGCGTGACAGCCGCAGATACCGCCGTATATTACTGTCATGTGT | |
| | TGATCTATGCCGGGTATCTTGCAATGGATTACTGGGGACAGGGCACTCTCGTAACAGTC | |
| | TCTTCAGCTAGCACCAAAGGACCTAGTGTTTTTCCTCTTGCCCCTTCCTCAAAGTCTAC | |
| | CTCTGGGGGGACAGCCGCTCTGGGCTGCCTGGTCAAGGATTATTTCCCAGAGCCTGTCA | |
| | CTGTCAGTTGGAACTCTGGAGCCTTGACTTCTGGTGTTCATACATTTCCTGCTGTCCTT | |
| | CAAAGCAGCGGCTTGTACTCATTGTCTTCTGTTGTGACAGTACCCTCAAGCAGCCTCGG | |
| | CACTCAGACATACATCTGCAATGTCAACCACAAACCCTCAAATACAAAGGTAGATAAAA | |
| | AAGTCGAACCAAAGTCTTGTGACAAAACTCACACGTGCCCACCGTGCCCAGCACCTGAA | |
| | CTCCTGGGGGGACCGTCAGTCTTCCTCTTCCCCCCAAAACCCAAGGACACCCTCATGAT | |
| | CTCCCGGACCCCTGAGGTCACATGCGTGGTGGTGGACGTGAGCCACGAAGACCCTGAGG | |
| | TCAAGTTCAACTGGTACGTGGACGGCGTGGAGGTGCATAATGCCAAGACAAAGCCGCGG | |
| | GAGGAGCAGTACAACAGCACGTACCGTGTGGTCAGCGTCCTCACCGTCCTGCACCAGGA | |
| | CTGGCTGAATGGCAAGGAGTACAAGTGCAAGGTCTCCAACAAAGCCCTCCCAGCCCCCA | |
| | TCGAGAAAACCATCTCCAAAGCCAAAGGGCAGCCCCGAGAACCACAGGTGTACACCCTG | |
| | CCCCCATGCCGGGAGGAGATGACCAAGAACCAGGTCAGCCTGTGGTGCCTGGTCAAAGG | |
| | CTTCTATCCCAGCGACATCGCCGTGGAGTGGGAGAGCAATGGGCAGCCGGAGAACAACT | |
| | ACAAGACCACGCCTCCCGTGCTGGACTCCGACGGCTCCTTCTTCCTCTACAGCAAGCTC | |
| | ACCGTGGACAAGAGCAGGTGGCAGCAGGGGAACGTCTTCTCATGCTCCGTGATGCATGA | |
| | GGCTCTGCACAACCACTACACGCAGAAGAGCCTCTCCCTGTCTCCGGGTAAA | |
| CEA03 LC | GACATACAAATGACCCAAAGCCCAAGCTCATTGAGCGCCTCCGTAGGAGATCGCGTAAC | 719 |
| | AATAACCTGCAGCGCCAGCAGTTCAGTAACTTATATGCACTGGTATCAGCAGAAGCCAG | |
| | GTAAGGCTCCCAAACTGTGGATATATAGCACCAGCAACCTGGCATCTGGTGTACCCTCT | |
| | CGATTTAGTGGCAGTGGTTCTGGAACAGACTATACCTTCACTATATCTTCTCTTCAGCC | |
| | TGAGGATATAGCCACTTACTATTGCCAGCAACGTTCCACTTACCCCCTGACTTTTGGGT | |
| | GTGGTACAAAGTTGGAAATAAAGCGTACGGTGGCAGCTCCCAGCGTTTTTATCTTTCCC | |
| | CCATCCGACGAGCAGCTCAAGAGTGGCACTGCCTCTGTAGTTTGTTTGCTGAATAACTT | |
| | CTATCCACGTGAAGCAAAAGTACAGTGGAAGGTCGATAATGCCCTTCAGAGCGGTAACA | |
| | GCCAAGAAAGTGTTACCGAGCAAGATTCCAAAGATTCCACTTACAGTCTGTCCAGCACA | |
| | TTGACACTGAGTAAGGCTGATTACGAAAAACACAAGGTGTACGCATGCGAGGTGACACA | |
| | CCAAGGTCTTTCATCTCCTGTAACTAAGAGCTTTAACCGGGGAGAATGTGGTGGTGGGG | |
| | GCAGCGGGGGCGGAGGTAGTGGAGGCGGCGGTAGTGAACCAAAGAGTAGTGACAAAACT | |
| | CACACGTGCCCACCGTGCCCAGCACCTGAACTCCTGGGGGGACCGTCAGTCTTCCTCTT | |
| | CCCCCCAAAACCCAAGGACACCCTCATGATCTCCCGGACCCCTGAGGTCACATGCGTGG | |
| | TGGTGGACGTGAGCCACGAAGACCCTGAGGTCAAGTTCAACTGGTACGTGGACGGCGTG | |
| | GAGGTGCATAATGCCAAGACAAAGCCGCGGGAGGAGCAGTACAACAGCACGTACCGTGT | |
| | GGTCAGCGTCCTCACCGTCCTGCACCAGGACTGGCTGAATGGCAAGGAGTACAAGTGCA | |
| | AGGTCTCCAACAAAGCCCTCCCAGCCCCCATCGAGAAAACCATCTCCAAAGCCAAAGGG | |
| | CAGCCCCGAGAACCACAGGTGTACACCCTGCCCCCATGCCGGGAGGAGATGACCAAGAA | |
| | CCAGGTCAGCCTGTGGTGCCTGGTCAAAGGCTTCTATCCCAGCGACATCGCCGTGGAGT | |
| | GGGAGAGCAATGGGCAGCCGGAGAACAACTACAAGACCACGCCTCCCGTGCTGGACTCC | |
| | GACGGCTCCTTCTTCCTCTACAGCAAGCTCACCGTGGACAAGAGCAGGTGGCAGCAGGG | |
| | GAACGTCTTCTCATGCTCCGTGATGCATGAGGCTCTGCACAACCACTACACGCAGAAGA | |
| | GCCTCTCCCTGTCTCCGGGTAAA | |
| CEA04 HC | GAAGTGCAGTTGGTGGAGTCTGGGGGCGGGTTGGTCCAACCAGGAGGCTCTCTGCGACT | 720 |
| | CTCTTGCGCAGCATCAGGCTTCAACATAAAGGACACATATATGCACTGGGTTCGGCAGG | |
| | CTCCCGGAAAATGTCTTGAATGGGTCGCCCGAATTGATCCTGCAAATGGGAACAGTAAA | |
| | TACGCAGATTCAGTTAAAGGCCGCTTCACTATCAGCGCAGATACATCCAAAAACACCGC | |
| | ATACCTTCAGATGAACTCACTCCGTGCAGAAGACACTGCAGTCTACTATTGCGCTCCCT | |
| | TCGGTTACTACGTCTCTGACTATGCAATGGCTTACTGGGGCCAAGGAACCTTGGTGACT | |
| | GTATCTTCTGCTAGCACCAAAGGACCTAGTGTTTTTCCTCTTGCCCCTTCCTCAAAGTC | |
| | TACCTCTGGGGGGACAGCCGCTCTGGGCTGCCTGGTCAAGGATTATTTCCCAGAGCCTG | |
| | TCACTGTCAGTTGGAACTCTGGAGCCTTGACTTCTGGTGTTCATACATTTCCTGCTGTC | |
| | CTTCAAAGCAGCGGCTTGTACTCATTGTCTTCTGTTGTGACAGTACCCTCAAGCAGCCT | |
| | CGGCACTCAGACATACATCTGCAATGTCAACCACAAACCCTCAAATACAAAGGTAGATA | |
| | AAAAAGTCGAACCAAAGTCTTGTGACAAAACTCACACGTGCCCACCGTGCCCAGCACCT | |
| | GAACTCCTGGGGGGACCGTCAGTCTTCCTCTTCCCCCCAAAACCCAAGGACACCCTCAT | |
| | GATCTCCCGGACCCCTGAGGTCACATGCGTGGTGGTGGACGTGAGCCACGAAGACCCTG | |
| | AGGTCAAGTTCAACTGGTACGTGGACGGCGTGGAGGTGCATAATGCCAAGACAAAGCCG | |
| | CGGGAGGAGCAGTACAACAGCACGTACCGTGTGGTCAGCGTCCTCACCGTCCTGCACCA | |
| | GGACTGGCTGAATGGCAAGGAGTACAAGTGCAAGGTCTCCAACAAAGCCCTCCCAGCCC | |
| | CCATCGAGAAAACCATCTCCAAAGCCAAAGGGCAGCCCCGAGAACCACAGGTGTACACC | |
| | CTGCCCCCATGCCGGGAGGAGATGACCAAGAACCAGGTCAGCCTGTGGTGCCTGGTCAA | |
| | AGGCTTCTATCCCAGCGACATCGCCGTGGAGTGGGAGAGCAATGGGCAGCCGGAGAACA | |
| | ACTACAAGACCACGCCTCCCGTGCTGGACTCCGACGGCTCCTTCTTCCTCTACAGCAAG | |
| | CTCACCGTGGACAAGAGCAGGTGGCAGCAGGGGAACGTCTTCTCATGCTCCGTGATGCA | |
| | TGAGGCTCTGCACAACCACTACACGCAGAAGAGCCTCTCCCTGTCTCCGGGTAAA | |
| CEA04 LC | GATATACAGCTTACACAATCACCCTCCTCCCTGTCTGCCTCCGTAGGAGATAGAGTAAC | 721 |
| | AATCACATGCAGAGCTGGCGAGAGTGTTGATATATTCGGTGTTGGATTTTTGCACTGGT | |
| | ACCAACAGAAACCAGGGAAAGCACCTAAGCTCTTGATTTATAGAGCTTCTAACCTTGAG | |
| | AGCGGGGTGCCTAGTAGGTTTTCTGGGTCAGGAAGTCGGACCGATTTTACTCTCACAAT | |
| | TTCATCCCTTCAGCCCGAAGACTTTGCAACCTACTACTGTCAGCAGACAAACGAAGACC | |
| | CCTATACATTCGGATGTGGTACAAAGGTGGAGATTAAACGTACGGTGGCAGCTCCCAGC | |
| | GTTTTTATCTTTCCCCCATCCGACGAGCAGCTCAAGAGTGGCACTGCCTCTGTAGTTTG | |
| | TTTGCTGAATAACTTCTATCCACGTGAAGCAAAAGTACAGTGGAAGGTCGATAATGCCC | |
| | TTCAGAGCGGTAACAGCCAAGAAAGTGTTACCGAGCAAGATTCCAAAGATTCCACTTAC | |
| | AGTCTGTCCAGCACATTGACACTGAGTAAGGCTGATTACGAAAAACACAAGGTGTACGC | |
| | ATGCGAGGTGACACACCAAGGTCTTTCATCTCCTGTAACTAAGAGCTTTAACCGGGGAG | |
| | AATGTGGTGGTGGGGGCAGCGGGGGCGGAGGTAGTGGAGGCGGCGGTAGTGAACCAAAG | |
| | AGTAGTGACAAAACTCACACGTGCCCACCGTGCCCAGCACCTGAACTCCTGGGGGGACC | |
| | GTCAGTCTTCCTCTTCCCCCCAAAACCCAAGGACACCCTCATGATCTCCCGGACCCCTG | |
| | AGGTCACATGCGTGGTGGTGGACGTGAGCCACGAAGACCCTGAGGTCAAGTTCAACTGG | |
| | TACGTGGACGGCGTGGAGGTGCATAATGCCAAGACAAAGCCGCGGGAGGAGCAGTACAA | |
| | CAGCACGTACCGTGTGGTCAGCGTCCTCACCGTCCTGCACCAGGACTGGCTGAATGGCA | |
| | AGGAGTACAAGTGCAAGGTCTCCAACAAAGCCCTCCCAGCCCCCATCGAGAAAACCATC | |
| | TCCAAAGCCAAAGGGCAGCCCCGAGAACCACAGGTGTACACCCTGCCCCCATGCCGGGA | |
| | GGAGATGACCAAGAACCAGGTCAGCCTGTGGTGCCTGGTCAAAGGCTTCTATCCCAGCG | |
| | ACATCGCCGTGGAGTGGGAGAGCAATGGGCAGCCGGAGAACAACTACAAGACCACGCCT | |
| | CCCGTGCTGGACTCCGACGGCTCCTTCTTCCTCTACAGCAAGCTCACCGTGGACAAGAG | |
| | CAGGTGGCAGCAGGGGAACGTCTTCTCATGCTCCGTGATGCATGAGGCTCTGCACAACC | |
| | ACTACACGCAGAAGAGCCTCTCCCTGTCTCCGGGTAAA | |

Table 76 below shows the polypeptide sequences of the heavy chain variable region (VH) and light chain variable region (VL) of the engineered antibodies targeting CEACAM5. Table 77 below shows the nucleotide sequences of the heavy chain variable region (VH) and light chain variable region (VL) of the engineered antibodies targeting CEACAM5.

TABLE 76

| | | | SEQ |
|---|---|---|---|
| Name | | Sequence | ID NO |
| CEA01 | VH | EVQLVESGGGVVQPGRSLRLSCSASGFDFTTYW | 819 |
| | | MSWVRQAPGKCLEWIGEIHPDSSTINYAPSLKD | |
| | | RFTISRDNAKNTLFLQMDSLRPEDTGVYFCASL | |
| | | YFGFPWFAYWGQGTPVTVSS | |
| | VL | DIQLTQSPSSLSASVGDRVTITCKASQDVGTSV | 820 |
| | | AWYQQKPGKAPKLLIYWTSTRHTGVPSRFSGSG | |
| | | SGTDFTFTISSLQPEDIATYYCQQYSLYRSFGC | |
| | | GTKVEIK | |
| CEA02 | VH | EVQLVESGGGLVQPGRSLRLSCAASGFTVSSYW | 821 |
| | | MHWVRQAPGKCLEWVGFILNKANGGTTEYAASV | |
| | | KGRFTISRDDSKNTLYLQMNSLRAEDTAVYYCA | |
| | | RDRGLRFYFDYWGQGTTVTVSS | |
| | VL | QAVLTQPASLSASPGASASLTCTLRRGINVGAY | 822 |
| | | SIYWYQQKPGSPPQYLLRYKSDSDKQQGSGVSS | |
| | | RFSASKDASANAGILLISGLQSEDEADYYCMIW | |
| | | HSGASAVFGCGTKLTVL | |
| CEA03 | VH | EVQLQQSGPGLVRPSQTLSLTCTASGFNIKDNY | 823 |
| | | MHWVRQPPGRCLEWIGWIDPENGDTEYAPKFRG | |
| | | RVTMLADTSKNQFSLRLSSVTAADTAVYYCHVL | |
| | | IYAGYLAMDYWGQGTLVTVSS | |
| | VL | DIQMTQSPSSLSASVGDRVTITCSASSSVTYMH | 824 |
| | | WYQQKPGKAPKLWIYSTSNLASGVPSRFSGSGS | |
| | | GTDYTFTISSLQPEDIATYYCQQRSTYPLTFGC | |
| | | GTKLEIK | |
| CEA04 | VH | EVQLVESGGGLVQPGGSLRLSCAASGFNIKDTY | 825 |
| | | MHWVRQAPGKCLEWVARIDPANGNSKYADSVKG | |
| | | RFTISADTSKNTAYLQMNSLRAEDTAVYYCAPF | |
| | | GYYVSDYAMAYWGQGTLVTVSS | |
| | VL | DIQLTQSPSSLSASVGDRVTITCRAGESVDIFG | 826 |
| | | VGFLHWYQQKPGKAPKLLIYRASNLESGVPSRF | |
| | | SGSGSRTDFTLTISSLQPEDFATYYCQQTNEDP | |
| | | YTFGCGTKVEIK | |

TABLE 77

| | | | SEQ |
|---|---|---|---|
| Name | | Sequence | ID NO |
| CEA01 | VH | GAAGTGCAACTCGTAGAATCCGGCGGGGGCGTCGTACAGCCCGGC | 827 |
| | | CGTTCTTTGAGACTTAGCTGTAGTGCTTCTGGGTTTGACTTCACTAC | |
| | | ATACTGGATGTCATGGGTAAGACAGGCACCTGGCAAGTGCCTTGA | |
| | | ATGGATCGGTGAAATCCATCCCGACAGCTCCACAATCAACTACGC | |
| | | CCCAAGTTTGAAAGACCGGTTCACCATATCTCGTGACAACGCCAA | |
| | | GAATACATTGTTCCTTCAGATGGATAGTCTTCGTCCAGAGGATACT | |
| | | GGGGTATATTTTTGTGCCAGCTTGTATTTTGGCTTCCCCTGGTTCGC | |
| | | TTATTGGGGCCAAGGTACACCCGTCACTGTCTCTTCT | |
| | VL | GACATCCAACTTACCCAGTCACCCTCATCTCTTAGTGCCTCTGTAG | 722 |
| | | GGGACCGAGTTACTATTACATGTAAAGCCAGTCAAGATGTTGGCA | |
| | | CCTCAGTAGCATGGTATCAACAAAAGCCTGGTAAAGCCCCAAAAC | |
| | | TGCTGATCTATTGGACAAGCACACGACATACAGGAGTGCCAAGTC | |
| | | GCTTCAGCGGTTCAGGTTCAGGCACAGATTTTACATTCACTATATC | |
| | | AAGCCTGCAACCCGAGGACATTGCCACATATTACTGCCAGCAATA | |
| | | TAGTCTGTATCGTAGCTTCGGATGTGGCACCAAGGTTGAAATTAAG | |
| CEA02 | VH | GAAGTTCAACTGGTGGAGTCTGGCGGCGGACTTGTCCAGCCAGGG | 723 |
| | | CGAAGCCTGCGTCTCTCATGCGCTGCCTCCGGTTTCACTGTTTCTTC | |
| | | ATACTGGATGCACTGGGTAAGACAGGCTCCTGGCAAGTGTCTTGA | |
| | | ATGGGTGGGCTTCATTTTGAACAAGGCAAACGGCGGGACTACCGA | |
| | | ATACGCTGCCAGCGTTAAGGGTCGATTCACCATCTCAAGGGATGAT | |
| | | TCTAAAAACACATTGTACCTTCAGATGAACTCTCTGAGGGCCGAGG | |
| | | ACACAGCAGTCTACTATTGCGCTAGAGATAGGGGGCTTAGATTCTA | |
| | | TTTTGACTACTGGGGGCAGGGAACAACTGTCACCGTTTCCAGT | |
| | VL | CAGGCAGTGCTTACTCAGCCTGCTTCACTCTCTGCCTCACCAGGAG | 724 |
| | | CATCTGCAAGCCTCACCTGCACATTGCGTCGAGGTATAAACGTAGG | |
| | | CGCTTACTCAATTTACTGGTACCAGCAGAAACCTGGGAGCCCACCT | |
| | | CAATACCTCCTCCGATACAAGAGCGATTCTGATAAGCAACAGGGC | |
| | | AGTGGTGTATCCAGCAGATTTTCCGCCAGCAAGGATGCAAGCGCT | |
| | | AATGCAGGTATTCTTCTCATTTCAGGCTTGCAAAGCGAGGACGAAG | |
| | | CAGACTACTACTGCATGATTTGGCACTCCGGTGCCTCCGCAGTTTT | |
| | | TGGCTGCGGAACAAAGCTTACAGTCCTT | |
| CEA03 | VH | GAGGTACAACTGCAGCAAAGCGGTCCAGGCCTTGTTCGGCCTAGC | 725 |
| | | CAGACTCTGTCACTGACCTGTACCGCAAGCGGTTTCAATATCAAGG | |
| | | ACAACTATATGCACTGGGTCCGCCAGCCTCCAGGACGCTGTTTGGA | |
| | | GTGGATAGGATGGATAGACCCAGAAAATGGGGATACCGAATACGC | |
| | | TCCAAAATTTCGTGGACGAGTTACCATGCTGGCCGATACATCCAAA | |
| | | AACCAGTTTTCTCTGAGGCTCTCCAGCGTGACAGCCGCAGATACCG | |
| | | CCGTATATTACTGTCATGTGTTGATCTATGCCGGGTATCTTGCAAT | |
| | | GGATTACTGGGGACAGGGCACTCTCGTAACAGTCTCTTCA | |
| | VL | GACATACAAATGACCCAAAGCCCAAGCTCATTGAGCGCCTCCGTA | 726 |
| | | GGAGATCGCGTAACAATAACCTGCAGCGCCAGCAGTTCAGTAACT | |
| | | TATATGCACTGGTATCAGCAGAAGCCAGGTAAGGCTCCCAAACTG | |
| | | TGGATATATAGCACCAGCAACCTGGCATCTGGTGTACCCTCTCGAT | |
| | | TTAGTGGCAGTGGTTCTGGAACAGACTATACCTTCACTATATCTTC | |
| | | TCTTCAGCCTGAGGATATAGCCACTTACTATTGCCAGCAACGTTCC | |
| | | ACTTACCCCCTGACTTTTGGGTGTGGTACAAAGTTGGAAATAAAG | |
| CEA04 | VH | GAAGTGCAGTTGGTGGAGTCTGGGGGCGGGTTGGTCCAACCAGGA | 727 |
| | | GGCTCTCTGCGACTCTCTTGCGCAGCATCAGGCTTCAACATAAAGG | |
| | | ACACATATATGCACTGGGTTCGGCAGGCTCCCGGAAAATGTCTTGA | |
| | | ATGGGTCGCCCGAATTGATCCTGCAAATGGGAACAGTAAATACGC | |
| | | AGATTCAGTTAAAGGCCGCTTCACTATCAGCGCAGATACATCCAA | |
| | | AAACACCGCATACCTTCAGATGAACTCACTCCGTGCAGAAGACAC | |
| | | TGCAGTCTACTATTGCGCTCCCTTCGGTTACTACGTCTCTGACTATG | |
| | | CAATGGCTTACTGGGGCCAAGGAACCTTGGTGACTGTATCTTCT | |
| | VL | GATATACAGCTTACACAATCACCCTCCTCCCTGTCTGCCTCCGTAG | 728 |
| | | GAGATAGAGTAACAATCACATGCAGAGCTGGCGAGAGTGTTGATA | |
| | | TATTCGGTGTTGGATTTTTGCACTGGTACCAACAGAAACCAGGGAA | |
| | | AGCACCTAAGCTCTTGATTTATAGAGCTTCTAACCTTGAGAGCGGG | |
| | | GTGCCTAGTAGGTTTTCTGGGTCAGGAAGTCGGACCGATTTTACTC | |
| | | TCACAATTTCATCCCTTCAGCCCGAAGACTTTGCAACCTACTACTG | |
| | | TCAGCAGACAAACGAAGACCCCTATACATTCGGATGTGGTACAAA | |
| | | GGTGGAGATTAAA | |

Table 78 below shows the heavy chain and light chain CDR sequences of the engineered antibodies targeting CEACAM5.

TABLE 78

| Name | CDR | Sequence | SEQ ID NO |
|---|---|---|---|
| CEA01 | CDR-H1 | TYWMS | 729 |
| | CDR-H2 | EIHPDSSTINYAPSLKD | 730 |
| | CDR-H3 | LYFGFPWFAY | 731 |
| | CDR-L1 | KASQDVGTSVA | 732 |
| | CDR-L2 | WTSTRHT | 733 |
| | CDR-L3 | QQYSLYRS | 734 |
| CEA02 | CDR-H1 | SYWMH | 735 |
| | CDR-H2 | FILNKANGGTTEYAASVKG | 736 |
| | CDR-H3 | DRGLRFYFDY | 737 |
| | CDR-L1 | TLRRGINVGAYSIY | 738 |
| | CDR-L2 | SDKQQGS | 739 |
| | CDR-L3 | MIWHSGASAV | 740 |
| CEA03 | CDR-H1 | DNYMH | 741 |
| | CDR-H2 | WIDPENGDTEYAPKFRG | 742 |
| | CDR-H3 | LIYAGYLAMDY | 743 |
| | CDR-L1 | SASSSVTYMH | 744 |
| | CDR-L2 | STSNLAS | 745 |
| | CDR-L3 | QQRSTYPLT | 746 |
| CEA04 | CDR-H1 | DTYMH | 747 |
| | CDR-H2 | RIDPANGNSKYADSVKG | 748 |
| | CDR-H3 | FGYYVSDYAMAY | 749 |
| | CDR-L1 | RAGESVDIFGVGFLH | 750 |
| | CDR-L2 | RASNLES | 751 |
| | CDR-L3 | QQTNEDPYT | 752 |

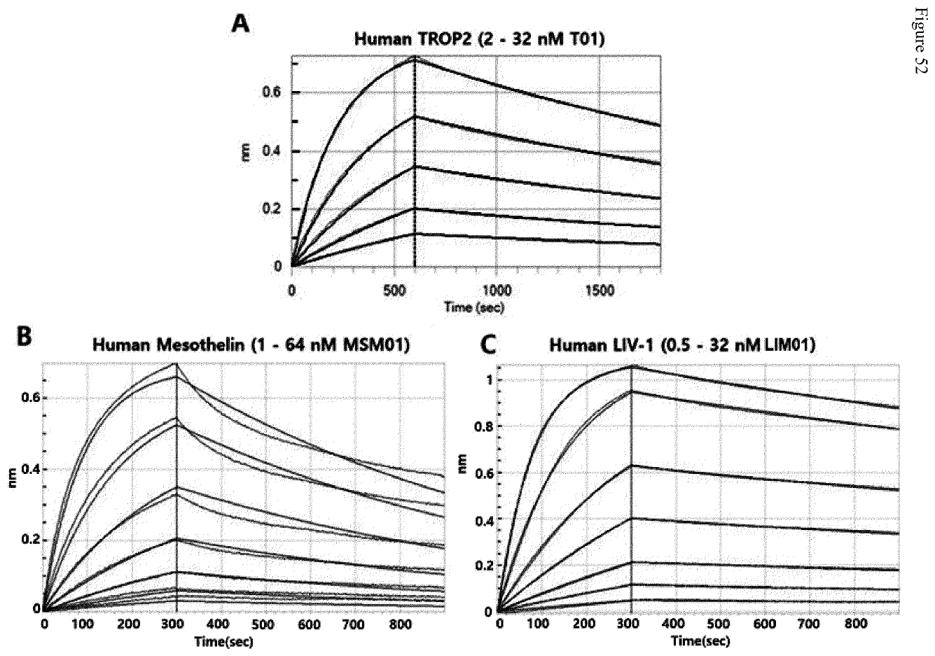

In order to analyze the binding constants, the human CEACAM5 recombinant protein (Sino Biologicals, 11077-H08H) was loaded onto the Anti-Penta-HIS (HIS1K) biosensor (Sartorius, 18-5120). Then, the antibodies were added in a binding reaction (600 seconds) and a dissociation reaction (1,200 seconds) at various concentrations, and their affinities for human CEACAM5 were calculated (FIG. 51, Table 79). Table 79 below illustrates the binding constants of the engineered antibodies targeting human CEACAM5.

TABLE 79

| Antibodies | K_{D} (nM) | Kₐ (1/Ms) | K_{d} (1/s) |
|---|---|---|---|
| CEA01 | 1.5740 | 1.76E+05 | 2.78E−04 |
| CEA02 | 19.2395 | 2.03E+04 | 3.90E−04 |
| CEA03 | 274.7780 | 1.13E+04 | 3.09E−03 |
| CEA04 | 1.7856 | 2.05E+05 | 3.66E−04 |

### Example 23. Design, Preparation and Analysis of Antibody Structure Targeting TROP2, Mesothelin or LIV-1

The variant light chain and heavy chain polypeptide sequences of the antibody T01 that specifically binds to the TROP2 protein are shown in Table 80. For T01I, expression vectors consisting of the sequences corresponding to Fc-Hole (SEQ ID NO: 7), T01 HC (SEQ ID NO: 753), and T01 LC (SEQ ID NO: 754) were co-transfected into EXPICHO-S™ (Gibco, A29127) (FIG. 41a, Table 80), and purification and analysis were performed in the same manner as described in Example 1. The variant light chain and heavy chain polypeptide sequences of the antibody MSM01 that specifically binds to the mesothelin protein are shown in Table 80. For MSM01, expression vectors consisting of the sequences corresponding to Fc-Hole (SEQ ID NO: 7), MSM01 HC (SEQ ID NO: 755), and MSM01 LC (SEQ ID NO: 756) were co-transfected into EXPICHO-S™ (Gibco, A29127) (FIG. 41a, Table 80), and purification and analysis were performed in the same manner as described in Example 1. The variant light chain and heavy chain polypeptide sequences of the antibody LIM01 that specifically binds to the LIV-1 protein are shown in Table 80. For LIM01, expression vectors consisting of the sequences corresponding to Fc-Hole (SEQ ID NO: 7), LIM01 HC (SEQ ID NO: 757), and LIM01 LC (SEQ ID NO: 758) were co-transfected into EXPICHO-S™ (Gibco, A29127) (FIG. 41a, Table 80), and purification and analysis were performed in the same manner as described in Example 1.

TABLE 80

| Target | | | SEQ |
|---|---|---|---|
| Antigen | Name | Sequence | ID NO |
| Human | T01 HC | QVQLQQSGSELKKPGASVKVSCKASGYTFTNYGMNWVKQAPGQC | 753 |
| TROP2 | | LKWMGWINTYTGEPTYTDDFKGRFAFSLDTSVSTAYLQISSLKAD | |
| | | DTAVYFCARGGFGSSYWYFDVWGQGSLVTVSSASTKGPSVFPLAP | |
| | | SSKSTSGGTAALGCLVKDYFPEPVTVSWNSGALTSGVHTFPAVLQS | |
| | | SGLYSLSSVVTVPSSSLGTQTYICNVNHKPSNTKVDKKVEPKSCDK | |
| | | THTCPPCPAPELLGGPSVFLFPPKPKDTLMISRTPEVTCVVVDVSHE | |
| | | DPEVKFNWYVDGVEVHNAKTKPREEQYNSTYRVVSVLTVLHQD | |
| | | WLNGKEYKCKVSNKALPAPIEKTISKAKGQPREPQVYTLPPCREEM | |
| | | TKNQVSLWCLVKGFYPSDIAVEWESNGQPENNYKTTPPVLDSDGS | |
| | | FFLYSKLTVDKSRWQQGNVFSCSVMHEALHNHYTQKSLSLSPGK | |
| | T01 LC | DIQLTQSPSSLSASVGDRVSITCKASQDVSIAVAWYQQKPGKAPKL | 754 |
| | | LIYSASYRYTGVPDRFSGSGSGTDFTLTISSLQPEDFAVYYCQQHYI | |
| | | TPLTFGCGTKVEIKRTVAAPSVFIFPPSDEQLKSGTASVVCLLNNFY | |
| | | PREAKVQWKVDNALQSGNSQESVTEQDSKDSTYSLSSTLTLSKAD | |
| | | YEKHKVYACEVTHQGLSSPVTKSFNRGECGGGGSGGGGSGGGGS | |
| | | EPKSSDKTHTCPPCPAPELLGGPSVFLFPPKPKDTLMISRTPEVTCV | |
| | | VVDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPCREEMTKNQVSLWCLVKGFYPSDIAVEWESNGQPENNYKTTPP | |
| | | VLDSDGSFFLYSKLTVDKSRWQQGNVFSCSVMHEALHNHYTQKS | |
| | | LSLSPGK | |
| Human | MSM01 | QVQLQQSGPELEKPGASVKISCKASGYSFTGYTMNWVKQSHGKCL | 755 |
| Mesothelin | HC | EWIGLITPYNGASSYNQKFRGKATLTVDKSSSTAYMDLLSLTSEDS | |
| | | AVYFCARGGYDGRGFDYWGQGTTVTVSSASTKGPSVFPLAPSSKS | |
| | | TSGGTAALGCLVKDYFPEPVTVSWNSGALTSGVHTFPAVLQSSGL | |
| | | YSLSSVVTVPSSSLGTQTYICNVNHKPSNTKVDKKVEPKSCDKTHT | |
| | | CPPCPAPELLGGPSVFLFPPKPKDTLMISRTPEVTCVVVDVSHEDPE | |
| | | VKFNWYVDGVEVHNAKTKPREEQYNSTYRVVSVLTVLHQDWLN | |
| | | GKEYKCKVSNKALPAPIEKTISKAKGQPREPQVYTLPPCREEMTKN | |
| | | QVSLWCLVKGFYPSDIAVEWESNGQPENNYKTTPPVLDSDGSFFL | |
| | | YSKLTVDKSRWQQGNVFSCSVMHEALHNHYTQKSLSLSPGK | |
| | MSM01 | DIELTQSPAIMSASPGEKVTMTCSASSSVSYMHWYQQKSGTSPKR | 756 |
| | LC | WIYDTSKLASGVPGRFSGSGSGNSYSLTISSVEAEDDATYYCQQWS | |
| | | KHPLTFGCGTKLEIKRTVAAPSVFIFPPSDEQLKSGTASVVCLLNNF | |
| | | YPREAKVQWKVDNALQSGNSQESVTEQDSKDSTYSLSSTLTLSKA | |
| | | DYEKHKVYACEVTHQGLSSPVTKSFNRGECGGGGSGGGGSGGGG | |
| | | SEPKSSDKTHTCPPCPAPELLGGPSVFLFPPKPKDTLMISRTPEVTCV | |
| | | VVDVSHEDPEVKFNWYVDGVEVHNAKTKPREEQYNSTYRVVSVL | |
| | | TVLHQDWLNGKEYKCKVSNKALPAPIEKTISKAKGQPREPQVYTL | |
| | | PPCREEMTKNQVSLWCLVKGFYPSDIAVEWESNGQPENNYKTTPP | |
| | | VLDSDGSFFLYSKLTVDKSRWQQGNVFSCSVMHEALHNHYTQKS | |
| | | LSLSPGK | |
| Human | LIM01 | EIQLQQSGPELMKPGASVKISCKASTYSFTRYFMHWVKQSHGECLE | 757 |
| LIV-1 | HC | WIGYIDPFNGGTGYNQKFKGKATLTVDKSSSTAYMHLSSLTSEDSA | |
| | | VYYCVTYGSDYFDYWGQGTTLTVSSASTKGPSVFPLAPSSKSTSGG | |
| | | TAALGCLVKDYFPEPVTVSWNSGALTSGVHTFPAVLQSSGLYSLSS | |
| | | VVTVPSSSLGTQTYICNVNHKPSNTKVDKKVEPKSCDKTHTCPPCP | |
| | | APELLGGPSVFLFPPKPKDTLMISRTPEVTCVVVDVSHEDPEVKFN | |
| | | WYVDGVEVHNAKTKPREEQYNSTYRVVSVLTVLHQDWLNGKEY | |
| | | KCKVSNKALPAPIEKTISKAKGQPREPQVYTLPPCREEMTKNQVSL | |
| | | WCLVKGFYPSDIAVEWESNGQPENNYKTTPPVLDSDGSFFLYSKLT | |
| | | VDKSRWQQGNVFSCSVMHEALHNHYTQKSLSLSPGK | |
| | LIM01 | DIVMTQPQKFMSTSVGDRVSVTCKASQNVETDVVWYQQKPGQPP | 758 |
| | LC | KALIYSASYRHSGVPDRFTGSGSGTNFTLTISTVQSEDLAEYFCQQY | |
| | | NNYPFTFGCGTKLEIKRTVAAPSVFIFPPSDEQLKSGTASVVCLLNN | |
| | | FYPREAKVQWKVDNALQSGNSQESVTEQDSKDSTYSLSSTLTLSK | |
| | | ADYEKHKVYACEVTHQGLSSPVTKSFNRGECGGGGSGGGGSGGG | |
| | | GSEPKSSDKTHTCPPCPAPELLGGPSVFLFPPKPKDTLMISRTPEVTC | |
| | | VVVDVSHEDPEVKFNWYVDGVEVHNAKTKPREEQYNSTYRVVSV | |
| | | LTVLHQDWLNGKEYKCKVSNKALPAPIEKTISKAKGQPREPQVYT | |
| | | LPPCREEMTKNQVSLWCLVKGFYPSDIAVEWESNGQPENNYKTTP | |
| | | PVLDSDGSFFLYSKLTVDKSRWQQGNVFSCSVMHEALHNHYTQKS | |
| | | LSLSPGK | |

Table 81 below shows the heavy chain and light chain nucleotide sequences of the engineered antibodies targeting TROP2, mesothelin, or LIV-1.

TABLE 81

| Target | | | SEQ |
|---|---|---|---|
| Ag | Name | Sequence | ID NO |
| Human | T01 HC | CAGGTGCAGCTCCAGCAGTCTGGTTCCGAGCTGAAGAAACCTGGGGCTTCAGTCAAAGTC | 759 |
| TROP2 | | TCTTGCAAGGCTTCAGGTTACACTTTCACCAATTATGGTATGAACTGGGTCAAGCAAGCT | |
| | | CCTGGTCAGTGTTTGAAGTGGATGGGGTGGATAAACACATATACTGGCGAACCTACATAC | |
| | | ACCGACGACTTCAAGGGACGCTTCGCCTTCTCTCTTGACACAAGTGTCTCAACAGCATAT | |
| | | CTCCAAATCAGTAGCCTTAAGGCCGACGACACAGCAGTTTATTTTTGCGCTAGGGGTGGA | |
| | | TTCGGATCTTCTTACTGGTATTTCGATGTCTGGGGACAAGGAAGTCTGGTCACAGTTTCC | |
| | | AGCGCTAGCACCAAAGGACCTAGTGTTTTTCCTCTTGCCCCTTCCTCAAAGTCTACCTCT | |
| | | GGGGGGACAGCCGCTCTGGGCTGCCTGGTCAAGGATTATTTCCCAGAGCCTGTCACTGTC | |
| | | AGTTGGAACTCTGGAGCCTTGACTTCTGGTGTTCATACATTTCCTGCTGTCCTTCAAAGC | |
| | | AGCGGCTTGTACTCATTGTCTTCTGTTGTGACAGTACCCTCAAGCAGCCTCGGCACTCAG | |
| | | ACATACATCTGCAATGTCAACCACAAACCCTCAAATACAAAGGTAGATAAAAAAGTCGAA | |
| | | CCAAAGTCTTGTGACAAAACTCACACGTGCCCACCGTGCCCAGCACCTGAACTCCTGGGG | |
| | | GGACCGTCAGTCTTCCTCTTCCCCCCAAAACCCAAGGACACCCTCATGATCTCCCGGACC | |
| | | CCTGAGGTCACATGCGTGGTGGTGGACGTGAGCCACGAAGACCCTGAGGTCAAGTTCAAC | |
| | | TGGTACGTGGACGGCGTGGAGGTGCATAATGCCAAGACAAAGCCGCGGGAGGAGCAGTAC | |
| | | AACAGCACGTACCGTGTGGTCAGCGTCCTCACCGTCCTGCACCAGGACTGGCTGAATGGC | |
| | | AAGGAGTACAAGTGCAAGGTCTCCAACAAAGCCCTCCCAGCCCCCATCGAGAAAACCATC | |
| | | TCCAAAGCCAAAGGGCAGCCCCGAGAACCACAGGTGTACACCCTGCCCCCATGCCGGGAG | |
| | | GAGATGACCAAGAACCAGGTCAGCCTGTGGTGCCTGGTCAAAGGCTTCTATCCCAGCGAC | |
| | | ATCGCCGTGGAGTGGGAGAGCAATGGGCAGCCGGAGAACAACTACAAGACCACGCCTCCC | |
| | | GTGCTGGACTCCGACGGCTCCTTCTTCCTCTACAGCAAGCTCACCGTGGACAAGAGCAGG | |
| | | TGGCAGCAGGGGAACGTCTTCTCATGCTCCGTGATGCATGAGGCTCTGCACAACCACTAC | |
| | | ACGCAGAAGAGCCTCTCCCTGTCTCCGGGTAAA | |
| | T01 LC | GACATACAACTCACACAATCTCCCTCTTCTTTGTCAGCTTCCGTTGGGGACAGGGTGTCA | 760 |
| | | ATTACTTGCAAAGCCTCTCAAGATGTTTCTATAGCTGTAGCCTGGTATCAACAGAAACCC | |
| | | GGAAAAGCTCCCAAGTTGTTGATTTATAGTGCTAGTTATAGGTACACTGGCGTGCCAGAT | |
| | | AGATTCAGTGGTAGCGGTTCTGGGACCGACTTTACCTTGACTATTTCTTCCCTGCAACCT | |
| | | GAGGATTTTGCCGTTTACTATTGCCAACAACATTATATTACTCCCCTTACTTTTGGGTGT | |
| | | GGGACCAAGGTAGAAATCAAGCGTACGGTGGCAGCTCCCAGCGTTTTTATCTTTCCCCCA | |
| | | TCCGACGAGCAGCTCAAGAGTGGCACTGCCTCTGTAGTTTGTTTGCTGAATAACTTCTAT | |
| | | CCACGTGAAGCAAAAGTACAGTGGAAGGTCGATAATGCCCTTCAGAGCGGTAACAGCCAA | |
| | | GAAAGTGTTACCGAGCAAGATTCCAAAGATTCCACTTACAGTCTGTCCAGCACATTGACA | |
| | | CTGAGTAAGGCTGATTACGAAAAACACAAGGTGTACGCATGCGAGGTGACACACCAAGGT | |
| | | CTTTCATCTCCTGTAACTAAGAGCTTTAACCGGGGAGAATGTGGTGGTGGGGGCAGCGGG | |
| | | GGCGGAGGTAGTGGAGGCGGCGGTAGTGAACCAAAGAGTAGTGACAAAACTCACACGTGC | |
| | | CCACCGTGCCCAGCACCTGAACTCCTGGGGGGACCGTCAGTCTTCCTCTTCCCCCCAAAA | |
| | | CCCAAGGACACCCTCATGATCTCCCGGACCCCTGAGGTCACATGCGTGGTGGTGGACGTG | |
| | | AGCCACGAAGACCCTGAGGTCAAGTTCAACTGGTACGTGGACGGCGTGGAGGTGCATAAT | |
| | | GCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAA | |
| | | GCCCTCCCAGCCCCCATCGAGAAAACCATCTCCAAAGCCAAAGGGCAGCCCCGAGAACCA | |
| | | CAGGTGTACACCCTGCCCCCATGCCGGGAGGAGATGACCAAGAACCAGGTCAGCCTGTGG | |
| | | TGCCTGGTCAAAGGCTTCTATCCCAGCGACATCGCCGTGGAGTGGGAGAGCAATGGGCAG | |
| | | CCGGAGAACAACTACAAGACCACGCCTCCCGTGCTGGACTCCGACGGCTCCTTCTTCCTC | |
| | | TACAGCAAGCTCACCGTGGACAAGAGCAGGTGGCAGCAGGGGAACGTCTTCTCATGCTCC | |
| | | GTGATGCATGAGGCTCTGCACAACCACTACACGCAGAAGAGCCTCTCCCTGTCTCCGGGT | |
| | | AAA | |
| Human | MSM01 HC | CAGGTTCAATTGCAGCAGTCTGGACCAGAATTGGAGAAGCCTGGCGCAAGCGTCAAAATA | 761 |
| Mesothelin | | TCTTGCAAGGCTTCAGGTTACAGTTTCACCGGATACACTATGAACTGGGTCAAGCAAAGC | |
| | | CACGGCAAGTGTCTTGAATGGATAGGATTGATTACTCCATACAATGGCGCTTCATCATAC | |
| | | AATCAGAAGTTTAGGGGCAAGGCAACTTTGACCGTGGATAAGTCATCATCTACCGCATAT | |
| | | ATGGACCTCTTGAGCCTCACAAGTGAAGACTCAGCTGTTTACTTTTGTGCCAGAGGAGGG | |
| | | TATGATGGGCGAGGATTCGACTATTGGGGTCAAGGAACCACCGTGACAGTAAGCTCTGCT | |
| | | AGCACCAAAGGACCTAGTGTTTTTCCTCTTGCCCCTTCCTCAAAGTCTACCTCTGGGGGG | |
| | | ACAGCCGCTCTGGGCTGCCTGGTCAAGGATTATTTCCCAGAGCCTGTCACTGTCAGTTGG | |
| | | AACTCTGGAGCCTTGACTTCTGGTGTTCATACATTTCCTGCTGTCCTTCAAAGCAGCGGC | |
| | | TTGTACTCATTGTCTTCTGTTGTGACAGTACCCTCAAGCAGCCTCGGCACTCAGACATAC | |
| | | ATCTGCAATGTCAACCACAAACCCTCAAATACAAAGGTAGATAAAAAAGTCGAACCAAAG | |
| | | TCTTGTGACAAAACTCACACGTGCCCACCGTGCCCAGCACCTGAACTCCTGGGGGGACCG | |
| | | TCAGTCTTCCTCTTCCCCCCAAAACCCAAGGACACCCTCATGATCTCCCGGACCCCTGAG | |
| | | GTCACATGCGTGGTGGTGGACGTGAGCCACGAAGACCCTGAGGTCAAGTTCAACTGGTAC | |
| | | GTGGACGGCGTGGAGGTGCATAATGCCAAGACAAAGCCGCGGGAGGAGCAGTACAACAGC | |
| | | ACGTACCGTGTGGTCAGCGTCCTCACCGTCCTGCACCAGGACTGGCTGAATGGCAAGGAG | |
| | | TACAAGTGCAAGGTCTCCAACAAAGCCCTCCCAGCCCCCATCGAGAAAACCATCTCCAAA | |
| | | GCCAAAGGGCAGCCCCGAGAACCACAGGTGTACACCCTGCCCCCATGCCGGGAGGAGATG | |
| | | ACCAAGAACCAGGTCAGCCTGTGGTGCCTGGTCAAAGGCTTCTATCCCAGCGACATCGCC | |
| | | GTGGAGTGGGAGAGCAATGGGCAGCCGGAGAACAACTACAAGACCACGCCTCCCGTGCTG | |
| | | GACTCCGACGGCTCCTTCTTCCTCTACAGCAAGCTCACCGTGGACAAGAGCAGGTGGCAG | |
| | | CAGGGGAACGTCTTCTCATGCTCCGTGATGCATGAGGCTCTGCACAACCACTACACGCAG | |
| | | AAGAGCCTCTCCCTGTCTCCGGGTAAA | |
| | MSM01 LC | GACATAGAGTTGACTCAATCTCCAGCAATCATGTCAGCCTCACCCGGAGAAAAGGTCACC | 762 |
| | | ATGACTTGTTCTGCAAGTTCCAGCGTTTCTTATATGCATTGGTACCAGCAGAAGTCAGGG | |
| | | ACTAGCCCTAAGAGATGGATTTACGATACCTCCAAACTGGCCTCCGGGGTGCCAGGCCGG | |
| | | TTTAGTGGCAGTGGAAGCGGTAACAGCTACTCTTTGACCATATCTAGCGTGGAAGCAGAG | |
| | | GACGACGCTACTTATTACTGTCAACAGTGGTCTAAGCACCCACTGACCTTCGGCTGTGGT | |
| | | ACAAAGCTCGAAATAAAACGTACGGTGGCAGCTCCCAGCGTTTTTATCTTTCCCCCATCC | |
| | | GACGAGCAGCTCAAGAGTGGCACTGCCTCTGTAGTTTGTTTGCTGAATAACTTCTATCCA | |
| | | CGTGAAGCAAAAGTACAGTGGAAGGTCGATAATGCCCTTCAGAGCGGTAACAGCCAAGAA | |
| | | AGTGTTACCGAGCAAGATTCCAAAGATTCCACTTACAGTCTGTCCAGCACATTGACACTG | |
| | | AGTAAGGCTGATTACGAAAAACACAAGGTGTACGCATGCGAGGTGACACACCAAGGTCTT | |
| | | TCATCTCCTGTAACTAAGAGCTTTAACCGGGGAGAATGTGGTGGTGGGGGCAGCGGGGGC | |
| | | GGAGGTAGTGGAGGCGGCGGTAGTGAACCAAAGAGTAGTGACAAAACTCACACGTGCCCA | |
| | | CCGTGCCCAGCACCTGAACTCCTGGGGGGACCGTCAGTCTTCCTCTTCCCCCCAAAACCC | |
| | | AAGGACACCCTCATGATCTCCCGGACCCCTGAGGTCACATGCGTGGTGGTGGACGTGAGC | |
| | | CACGAAGACCCTGAGGTCAAGTTCAACTGGTACGTGGACGGCGTGGAGGTGCATAATGCC | |
| | | AAGACAAAGCCGCGGGAGGAGCAGTACAACAGCACGTACCGTGTGGTCAGCGTCCTCACC | |
| | | GTCCTGCACCAGGACTGGCTGAATGGCAAGGAGTACAAGTGCAAGGTCTCCAACAAAGCC | |
| | | CTCCCAGCCCCCATCGAGAAAACCATCTCCAAAGCCAAAGGGCAGCCCCGAGAACCACAG | |
| | | GTGTACACCCTGCCCCCATGCCGGGAGGAGATGACCAAGAACCAGGTCAGCCTGTGGTGC | |
| | | CTGGTCAAAGGCTTCTATCCCAGCGACATCGCCGTGGAGTGGGAGAGCAATGGGCAGCCG | |
| | | GAGAACAACTACAAGACCACGCCTCCCGTGCTGGACTCCGACGGCTCCTTCTTCCTCTAC | |
| | | AGCAAGCTCACCGTGGACAAGAGCAGGTGGCAGCAGGGGAACGTCTTCTCATGCTCCGTG | |
| | | ATGCATGAGGCTCTGCACAACCACTACACGCAGAAGAGCCTCTCCCTGTCTCCGGGTAAA | |
| Human | LIM01 HC | GAGATACAGTTGCAGCAAAGCGGACCCGAGCTTATGAAACCAGGAGCTAGTGTGAAAATT | 763 |
| LIV-1 | | AGCTGCAAGGCTAGCACCTACTCTTTTACTCGCTATTTTATGCATTGGGTTAAACAGTCA | |
| | | CATGGTGAGTGTTTGGAATGGATCGGGTACATTGATCCCTTTAATGGAGGGACTGGCTAC | |
| | | AACCAGAAGTTTAAAGGAAAAGCCACTCTCACTGTTGACAAAAGTAGTAGTACAGCATAT | |
| | | ATGCACCTCAGTTCCCTTACCAGTGAAGATAGCGCAGTTTACTATTGTGTCACTTACGGA | |
| | | TCAGACTACTTCGACTATTGGGGACAGGGTACAACCCTTACAGTCTCCAGTGCTAGCACC | |
| | | AAAGGACCTAGTGTTTTTCCTCTTGCCCCTTCCTCAAAGTCTACCTCTGGGGGGACAGCC | |
| | | GCTCTGGGCTGCCTGGTCAAGGATTATTTCCCAGAGCCTGTCACTGTCAGTTGGAACTCT | |
| | | GGAGCCTTGACTTCTGGTGTTCATACATTTCCTGCTGTCCTTCAAAGCAGCGGCTTGTAC | |
| | | TCATTGTCTTCTGTTGTGACAGTACCCTCAAGCAGCCTCGGCACTCAGACATACATCTGC | |
| | | AATGTCAACCACAAACCCTCAAATACAAAGGTAGATAAAAAAGTCGAACCAAAGTCTTGT | |
| | | GACAAAACTCACACGTGCCCACCGTGCCCAGCACCTGAACTCCTGGGGGGACCGTCAGTC | |
| | | TTCCTCTTCCCCCCAAAACCCAAGGACACCCTCATGATCTCCCGGACCCCTGAGGTCACA | |
| | | TGCGTGGTGGTGGACGTGAGCCACGAAGACCCTGAGGTCAAGTTCAACTGGTACGTGGAC | |
| | | GGCGTGGAGGTGCATAATGCCAAGACAAAGCCGCGGGAGGAGCAGTACAACAGCACGTAC | |
| | | CGTGTGGTCAGCGTCCTCACCGTCCTGCACCAGGACTGGCTGAATGGCAAGGAGTACAAG | |
| | | TGCAAGGTCTCCAACAAAGCCCTCCCAGCCCCCATCGAGAAAACCATCTCCAAAGCCAAA | |
| | | GGGCAGCCCCGAGAACCACAGGTGTACACCCTGCCCCCATGCCGGGAGGAGATGACCAAG | |
| | | AACCAGGTCAGCCTGTGGTGCCTGGTCAAAGGCTTCTATCCCAGCGACATCGCCGTGGAG | |
| | | TGGGAGAGCAATGGGCAGCCGGAGAACAACTACAAGACCACGCCTCCCGTGCTGGACTCC | |
| | | GACGGCTCCTTCTTCCTCTACAGCAAGCTCACCGTGGACAAGAGCAGGTGGCAGCAGGGG | |
| | | AACGTCTTCTCATGCTCCGTGATGCATGAGGCTCTGCACAACCACTACACGCAGAAGAGC | |
| | | CTCTCCCTGTCTCCGGGTAAA | |
| | LIM01 LC | GACATAGTGATGACCCAACCACAAAAGTTTATGTCAACCTCCGTTGGAGATCGTGTTTCT | 764 |
| | | GTCACTTGTAAGGCATCACAGAATGTAGAGACAGACGTAGTCTGGTATCAGCAGAAACCT | |
| | | GGTCAGCCACCTAAAGCACTCATCTACAGTGCAAGTTACCGACATTCTGGCGTACCCGAC | |
| | | AGATTTACTGGGTCTGGTTCTGGTACAAATTTCACTCTCACCATCTCAACCGTCCAATCA | |
| | | GAAGACTTGGCCGAGTATTTTTGCCAGCAATATAACAATTATCCCTTTACATTCGGATGT | |
| | | GGGACCAAACTCGAAATCAAACGTACGGTGGCAGCTCCCAGCGTTTTTATCTTTCCCCCA | |
| | | TCCGACGAGCAGCTCAAGAGTGGCACTGCCTCTGTAGTTTGTTTGCTGAATAACTTCTAT | |
| | | CCACGTGAAGCAAAAGTACAGTGGAAGGTCGATAATGCCCTTCAGAGCGGTAACAGCCAA | |
| | | GAAAGTGTTACCGAGCAAGATTCCAAAGATTCCACTTACAGTCTGTCCAGCACATTGACA | |
| | | CTGAGTAAGGCTGATTACGAAAAACACAAGGTGTACGCATGCGAGGTGACACACCAAGGT | |
| | | CTTTCATCTCCTGTAACTAAGAGCTTTAACCGGGGAGAATGTGGTGGTGGGGGCAGCGGG | |
| | | GGCGGAGGTAGTGGAGGCGGCGGTAGTGAACCAAAGAGTAGTGACAAAACTCACACGTGC | |
| | | CCACCGTGCCCAGCACCTGAACTCCTGGGGGGACCGTCAGTCTTCCTCTTCCCCCCAAAA | |
| | | CCCAAGGACACCCTCATGATCTCCCGGACCCCTGAGGTCACATGCGTGGTGGTGGACGTG | |
| | | AGCCACGAAGACCCTGAGGTCAAGTTCAACTGGTACGTGGACGGCGTGGAGGTGCATAAT | |
| | | GCCAAGACAAAGCCGCGGGAGGAGCAGTACAACAGCACGTACCGTGTGGTCAGCGTCCTC | |
| | | ACCGTCCTGCACCAGGACTGGCTGAATGGCAAGGAGTACAAGTGCAAGGTCTCCAACAAA | |
| | | GCCCTCCCAGCCCCCATCGAGAAAACCATCTCCAAAGCCAAAGGGCAGCCCCGAGAACCA | |
| | | CAGGTGTACACCCTGCCCCCATGCCGGGAGGAGATGACCAAGAACCAGGTCAGCCTGTGG | |
| | | TGCCTGGTCAAAGGCTTCTATCCCAGCGACATCGCCGTGGAGTGGGAGAGCAATGGGCAG | |
| | | CCGGAGAACAACTACAAGACCACGCCTCCCGTGCTGGACTCCGACGGCTCCTTCTTCCTC | |
| | | TACAGCAAGCTCACCGTGGACAAGAGCAGGTGGCAGCAGGGGAACGTCTTCTCATGCTCC | |
| | | GTGATGCATGAGGCTCTGCACAACCACTACACGCAGAAGAGCCTCTCCCTGTCTCCGGGT | |
| | | AAA | |

Table 82 below shows the polypeptide sequences of the heavy chain variable region (VH) and light chain variable region (VL) of the engineered antibodies targeting TROP2, mesothelin, or LIV-1. Table 83 below shows the nucleotide sequences of the heavy chain variable region (VH) and light chain variable region (VL) of the engineered antibodies targeting TROP2, mesothelin, or LIV-1.

TABLE 82

| | | | | SEQ |
|---|---|---|---|---|
| Antigen | Name | | Sequence | ID NO |
| Human | T01 | VH | QVQLQQSGSELKKPGASVKV | 765 |
| TROP2 | | | SCKASGYTFTNYGMNWVKQA | |
| | | | PGQCLKWMGWINTYTGEPTY | |
| | | | TDDFKGRFAFSLDTSVSTAY | |
| | | | LQISSLKADDTAVYFCARGG | |
| | | | FGSSYWYFDVWGQGSLVTVS | |
| | | | S | |
| | | VL | DIQLTQSPSSLSASVGDRVS | 766 |
| | | | ITCKASQDVSIAVAWYQQKP | |
| | | | GKAPKLLIYSASYRYTGVPD | |
| | | | RFSGSGSGTDFTLTISSLQP | |
| | | | EDFAVYYCQQHYITPLTFGC | |
| | | | GTKVEIK | |
| Human | MSM01 | VH | QVQLQQSGPELEKPGASVKI | 767 |
| Mesothelin | | | SCKASGYSFTGYTMNWVKQS | |
| | | | HGKCLEWIGLITPYNGASSY | |
| | | | NQKFRGKATLTVDKSSSTAY | |
| | | | MDLLSLTSEDSAVYFCARGG | |
| | | | YDGRGFDYWGQGTTVTVSS | |
| | | VL | DIELTQSPAIMSASPGEKVT | 768 |
| | | | MTCSASSSVSYMHWYQQKSG | |
| | | | TSPKRWIYDTSKLASGVPGR | |
| | | | FSGSGSGNSYSLTISSVEAE | |
| | | | DDATYYCQQWSKHPLTFGCG | |
| | | | TKLEIK | |
| Human | LIM01 | VH | EIQLQQSGPELMKPGASVKI | 769 |
| LIV-1 | | | SCKASTYSFTRYFMHWVKQS | |
| | | | HGECLEWIGYIDPFNGGTGY | |
| | | | NQKFKGKATLTVDKSSSTAY | |
| | | | MHLSSLTSEDSAVYYCVTYG | |
| | | | SDYFDYWGQGTTLTVSS | |
| | | VL | DIVMTQPQKFMSTSVGDRVS | 770 |
| | | | VTCKASQNVETDVVWYQQKP | |
| | | | GQPPKALIYSASYRHSGVPD | |
| | | | RFTGSGSGTNFTLTISTVQS | |
| | | | EDLAEYFCQQYNNYPFTFGC | |
| | | | GTKLEIK | |

TABLE 83

| | | | | SEQ |
|---|---|---|---|---|
| Antigen | Name | | Sequence | ID NO |
| Human | T01 | VH | CAGGTGCAGCTCCAGCAGTCTGGTTCCGAGCTGAAGAAAC | 771 |
| TROP2 | | | CTGGGGCTTCAGTCAAAGTCTCTTGCAAGGCTTCAGGTTA | |
| | | | CACTTTCACCAATTATGGTATGAACTGGGTCAAGCAAGCT | |
| | | | CCTGGTCAGTGTTTGAAGTGGATGGGGTGGATAAACACAT | |
| | | | ATACTGGCGAACCTACATACACCGACGACTTCAAGGGACG | |
| | | | CTTCGCCTTCTCTCTTGACACAAGTGTCTCAACAGCATATC | |
| | | | TCCAAATCAGTAGCCTTAAGGCCGACGACACAGCAGTTTA | |
| | | | TTTTTGCGCTAGGGGTGGATTCGGATCTTCTTACTGGTATT | |
| | | | TCGATGTCTGGGGACAAGGAAGTCTGGTCACAGTTTCCAG | |
| | | | C | |
| | | VL | GACATACAACTCACACAATCTCCCTCTTCTTTGTCAGCTTC | 772 |
| | | | CGTTGGGGACAGGGTGTCAATTACTTGCAAAGCCTCTCAA | |
| | | | GATGTTTCTATAGCTGTAGCCTGGTATCAACAGAAACCCG | |
| | | | GAAAAGCTCCCAAGTTGTTGATTTATAGTGCTAGTTATAG | |
| | | | GTACACTGGCGTGCCAGATAGATTCAGTGGTAGCGGTTCT | |
| | | | GGGACCGACTTTACCTTGACTATTTCTTCCCTGCAACCTGA | |
| | | | GGATTTTGCCGTTTACTATTGCCAACAACATTATATTACTC | |
| | | | CCCTTACTTTTGGGTGTGGGACCAAGGTAGAAATCAAG | |
| Human | MSM01 | VH | CAGGTTCAATTGCAGCAGTCTGGACCAGAATTGGAGAAGC | 773 |
| Mesothelin | | | CTGGCGCAAGCGTCAAAATATCTTGCAAGGCTTCAGGTTA | |
| | | | CAGTTTCACCGGATACACTATGAACTGGGTCAAGCAAAGC | |
| | | | CACGGCAAGTGTCTTGAATGGATAGGATTGATTACTCCAT | |
| | | | ACAATGGCGCTTCATCATACAATCAGAAGTTTAGGGGCAA | |
| | | | GGCAACTTTGACCGTGGATAAGTCATCATCTACCGCATAT | |
| | | | ATGGACCTCTTGAGCCTCACAAGTGAAGACTCAGCTGTTT | |
| | | | ACTTTTGTGCCAGAGGAGGGTATGATGGGCGAGGATTCGA | |
| | | | CTATTGGGGTCAAGGAACCACCGTGACAGTAAGCTCT | |
| | | VL | GACATAGAGTTGACTCAATCTCCAGCAATCATGTCAGCCT | 774 |
| | | | CACCCGGAGAAAAGGTCACCATGACTTGTTCTGCAAGTTC | |
| | | | CAGCGTTTCTTATATGCATTGGTACCAGCAGAAGTCAGGG | |
| | | | ACTAGCCCTAAGAGATGGATTTACGATACCTCCAAACTGG | |
| | | | CCTCCGGGGTGCCAGGCCGGTTTAGTGGCAGTGGAAGCGG | |
| | | | TAACAGCTACTCTTTGACCATATCTAGCGTGGAAGCAGAG | |
| | | | GACGACGCTACTTATTACTGTCAACAGTGGTCTAAGCACC | |
| | | | CACTGACCTTCGGCTGTGGTACAAAGCTCGAAATAAAA | |
| Human | LIM01 | VH | GAGATACAGTTGCAGCAAAGCGGACCCGAGCTTATGAAA | 775 |
| LIV-1 | | | CCAGGAGCTAGTGTGAAAATTAGCTGCAAGGCTAGCACCT | |
| | | | ACTCTTTTACTCGCTATTTTATGCATTGGGTTAAACAGTCA | |
| | | | CATGGTGAGTGTTTGGAATGGATCGGGTACATTGATCCCT | |
| | | | TTAATGGAGGGACTGGCTACAACCAGAAGTTTAAAGGAA | |
| | | | AAGCCACTCTCACTGTTGACAAAAGTAGTAGTACAGCATA | |
| | | | TATGCACCTCAGTTCCCTTACCAGTGAAGATAGCGCAGTTT | |
| | | | ACTATTGTGTCACTTACGGATCAGACTACTTCGACTATTGG | |
| | | | GGACAGGGTACAACCCTTACAGTCTCCAGT | |
| | | VL | GACATAGTGATGACCCAACCACAAAAGTTTATGTCAACCT | 776 |
| | | | CCGTTGGAGATCGTGTTTCTGTCACTTGTAAGGCATCACAG | |
| | | | AATGTAGAGACAGACGTAGTCTGGTATCAGCAGAAACCTG | |
| | | | GTCAGCCACCTAAAGCACTCATCTACAGTGCAAGTTACCG | |
| | | | ACATTCTGGCGTACCCGACAGATTTACTGGGTCTGGTTCTG | |
| | | | GTACAAATTTCACTCTCACCATCTCAACCGTCCAATCAGA | |
| | | | AGACTTGGCCGAGTATTTTTGCCAGCAATATAACAATTAT | |
| | | | CCCTTTACATTCGGATGTGGGACCAAACTCGAAATCAAA | |

Table 84 below shows the heavy chain and light chain CDR sequences of the engineered antibodies targeting TROP2, mesothelin, or LIV-1.

TABLE 84

| | | | | SEQ |
|---|---|---|---|---|
| Antigen | Name | CDR | Sequence | ID NO |
| Human | CEA01 | CDR-H1 | NYGMN | 260 |
| TROP2 | | CDR-H2 | WINTYTGEPTYTDDFKG | 261 |
| | | CDR-H3 | GGFGSSYWYFDV | 262 |
| | | CDR-L1 | KASQDVSIAVA | 263 |
| | | CDR-L2 | SASYRYT | 264 |
| | | CDR-L3 | QQHYITPLT | 265 |
| Human | MSM01 | CDR-H1 | GYTMN | 777 |
| Mesothelin | | CDR-H2 | LITPYNGASSYNQKFRG | 778 |
| | | CDR-H3 | GGYDGRGFDY | 779 |
| | | CDR-L1 | SASSSVSYMH | 780 |
| | | CDR-L2 | DTSKLAS | 781 |
| | | CDR-L3 | QQWSKHPLT | 782 |
| Human | LIM01 | CDR-H1 | RYFMH | 783 |
| LIV-1 | | CDR-H2 | YIDPFNGGTGYNQKFKG | 784 |
| | | CDR-H3 | YGSDYFDY | 785 |
| | | CDR-L1 | KASQNVETDVV | 786 |
| | | CDR-L2 | SASYRHS | 787 |
| | | CDR-L3 | QQYNNYP | 788 |

In order to analyze the binding constants, the human TROP2 recombinant protein (Sino Biologicals, 10428-H08H), the human mesothelin recombinant protein (Sino Biologicals, 13128-H08H) or the human LIV-1 recombinant protein (Acro biosystems, LV1-H5223) was loaded onto the Anti-Penta-HIS (HIS1K) biosensor (Sartorius, 18-5120). Then, the antibodies were added in a binding reaction and a dissociation reaction at various concentrations, and the affinities of each antibody for the human antigen was calculated (FIG. 52, Table 85). Table 85 below illustrates the binding constants of the engineered antibodies targeting TROP2, mesothelin, or LIV-1.

TABLE 85

| Antigens | Antibodies | K_{D} (nM) | Kₐ (1/Ms) | K_{d} (1/s) |
|---|---|---|---|---|
| Human TROP2 | T01 | 1.2008 | 2.67E+05 | 3.21E−04 |
| Human Mesothelin | MSM01 | 7.1771 | 1.59E+05 | 1.14E−03 |
| Human LIV-1 | LIM01 | 0.8072 | 3.84E+05 | 3.10E−04 |

1. A fusion protein comprising:
(a) an antigen-binding site consisting of a first polypeptide comprising at least one complementarity-determining region (CDR) sequence and a second polypeptide comprising at least one complementarity-determining region (CDR) sequence, wherein the first polypeptide and the second polypeptide form a dimer, and the antigen-binding site is capable of specifically binding to a target antigen,
(b) a first Fc domain or a variant thereof that is a dimer consisting of two polypeptide sequences, one of which is bound to the first polypeptide of the antigen-binding site, and
(c) a second Fc domain or a variant thereof that is a dimer consisting of two polypeptide sequences, one of which is bound to the second polypeptide of the antigen-binding site.

2. The fusion protein according to claim 1, wherein the first polypeptide of the antigen-binding site comprises CDR1, CDR2, and CDR3 of an antibody heavy chain, and the second polypeptide of the antigen-binding site comprises CDR1, CDR2, and CDR3 of an antibody light chain.

3. The fusion protein according to claim 2, wherein the first polypeptide of the antigen-binding site further comprises a CH1 region of an antibody heavy chain, and/or the second polypeptide of the antigen-binding site further comprises a constant region of an antibody light chain.

4. The fusion protein according to claim 1, wherein the antigen-binding site specifically binds to a protein expressed on the cell surface.

5. The fusion protein according to claim 1, wherein the antigen-binding site specifically binds to a cancer antigen.

6. The fusion protein according to claim 1, wherein the fusion protein induces improved antitumor activity compared to an IgG-based antibody of a conventional structure having the same antigen-binding site.

7. The fusion protein according to claim 1, wherein the antigen-binding site specifically binds to any one selected from the group consisting of PD-L1, EGFR, EGFRvIII, BCMA, CD22, CD25, CD30, CD33, CD37, CD38, CD52, CD56, CD123, c-Met, DLL3, DR4, DR5, GD2, nectin-4, RANKL, SLAMF7, Trop-2, LIV-1, claudin 18.2, IL13α2, CD3, HER2, HER3, FGFR2, FGFR3, GPC3, ROR1, Folα, CD20, CD19, CTLA-4, VEGFR, NCAM1, ICAM-1, ICAM-2, CEACAM5, CEACAM6, carcinoembryonic antigen (CEA), CA-125, alphafetoprotein (AFP), MUC-1, MUC-16, PSMA, PSCA, epithelial tumor antigen (ETA), melanoma-associated antigen (MAGE), immature laminin receptor, TAG-72, HPV E6/E7, BING-4, calcium-activated chloride channel 2, cyclin-B1, 9D7, Ep-CAM, EphA2, EphA3, mesothelin, SAP-1, survivin, and virus-derived antigens.

8. The fusion protein according to claim 1, wherein the first Fc domain and the second Fc domain are each a wild type Fc domain or an Fc domain variant.

9. The fusion protein according to claim 8, wherein the Fc region is an Fc region of IgG, IgA, IgE, IgD, or IgM or a variant thereof.

10. The fusion protein according to claim 8, wherein
the first Fc domain variant and the second Fc domain variant each independently comprise a knob variant or a hole variant that promotes the formation of an Fc heterodimer (heterodimeric Fc); and/or
the first Fc domain variant and the second Fc domain variant each independently comprise a variant that promotes the formation of a heterodimer by electrostatic steering mechanism.

11. The fusion protein according to claim 1, wherein the fusion protein comprises polypeptides of the following structural formulas (I), (II), (III), and (IV):
N′—X-(L1)n-A-C′ (I);
N′—Y-(L2)m-B—C′ (II);
N′—C—C′ (III); and
N′-D-C′ (IV)
wherein, in the structural formulas (I), (II), (III), and (IV),
N′ is the N-terminus of each polypeptide,
C′ is the C-terminus of each polypeptide,
refers to a linkage,
A, B, C, and D are monomeric polypeptide sequences of an Fc domain each comprising the CH2 and CH3 regions of an immunoglobulin, and optionally further comprising CH4 and/or a hinge sequence, wherein A forms a dimer with one of C or D to form the first Fc domain (b), and B forms a dimer with the remaining one of C or D to form the second Fc domain (c);
L1 and L2 are each peptide linker,
n and m are each independently 0 or 1,
X is a first polypeptide sequence of the antigen-binding site, which comprises heavy chain CDR1, CDR2, and CDR3 sequences of an antibody that specifically binds to a first antigen, or a heavy chain variable region of an antibody that specifically binds to a first antigen;
Y is a second polypeptide sequence of the antigen-binding site, which comprises light chain CDR1, CDR2, and CDR3 sequences of an antibody that specifically binds to a first antigen, or a light chain variable region of an antibody that specifically binds to a first antigen; and
X and Y pair with each other to form the antigen-binding site (a) that specifically binds to an antigen.

12. The fusion protein according to claim 11, wherein
X in the structural formula (I) further comprises a heavy chain CH1 region, and/or
Y in the structural formula (II) further comprises a light chain constant region.

13. The fusion protein according to claim 1, wherein the fusion protein comprises polypeptides of the following structural formulas (I′), (II′), (III), and (IV):
N′—VD1-(L3)p-X-(L1)n-A-C′ (I′);
N′—VD2-(L4)q-Y-(L2)m-B—C′ (II′);
N′—C—C′ (III); and
N′-D-C′ (IV)
wherein, in the structural formulas (I′), (II′), (III), and (IV),
N′ is the N-terminus of each polypeptide,
C′ is the C-terminus of each polypeptide,
refers to a linkage,
A, B, C, and D are monomeric polypeptide sequences of an Fc domain each comprising the CH2 and CH3 regions of an immunoglobulin, and optionally further comprising CH4 and/or a hinge sequence, wherein A forms a dimer with one of C or D to form the first Fc domain (b), and B forms a dimer with the remaining one of C or D to form the second Fc domain (c);
L1, L2, L3, and L4 are each peptide linker,
n, m, p, and q are each 0 or 1,
VD1 consists of a heavy chain or light chain variable region of an antibody that specifically binds to an antigen, or CDR1, CDR2, and CDR3 of an antibody heavy chain or light chain;
VD2 consists of a light chain or heavy chain variable region of an antibody that specifically binds to an antigen, or CDR1, CDR2, and CDR3 of an antibody heavy chain or light chain;
VD1 and VD2 pair with each other to form a second antibody variable region that specifically binds to a second antigen,
X comprises a heavy chain or light chain variable region of an antibody that specifically binds to an antigen, or CDR1, CDR2, and CDR3 of an antibody heavy chain or light chain;
Y comprises a light chain or heavy chain variable region of an antibody that specifically binds to an antigen, or CDR1, CDR2, and CDR3 of an antibody heavy chain or light chain; and
X and Y pair with each other to form a first antibody variable region that specifically binds to a first antigen, and
VD1-(L3)p-X forms a first polypeptide sequence of the antigen-binding site (a), and VD2-(L4)q-Y forms a second polypeptide sequence of the antigen-binding site (a).

14. The fusion protein according to claim 13, wherein
the heavy chain variable region further comprises a heavy chain CH1 region, and
the light chain variable region further comprises a light chain constant region.

15. The fusion protein according to claim 11, wherein
the Fc domain monomer comprises a knob variant or a hole variant that promotes the formation of an Fc heterodimer (heterodimeric Fc); or
the Fc domain monomer comprises a variant that promotes the formation of a heterodimer by electrostatic steering mechanism.

16. The fusion protein according to claim 12, wherein the binding between X and Y is achieved
i) through a disulfide bond formed by Cys present in CH1 and a light chain constant region,
ii) through a disulfide bond formed by Cys present in a heavy chain variable region and a light chain variable region, or
iii) through a disulfide bond formed by Cys present in CH1 and a light chain constant region, and a disulfide bond formed by Cys present in a heavy chain variable region and a light chain variable region.

17. The fusion protein according to claim 14, wherein the binding between X and Y further comprises, in addition to a disulfide bond present between CH1233 and CL214 based on Kabat numbering system,
i) a disulfide bond present between VH105 and VL43;
ii) a disulfide bond present between VH44 and VL100; or
iii) a disulfide bond present between CH₁122 and CL121.

18. (canceled)

19. The method according to claim 21, wherein the cancer is any one selected from the group consisting of gastric cancer, liver cancer, lung cancer, large intestine cancer, breast cancer, prostate cancer, gallbladder cancer, bladder cancer, kidney cancer, esophageal cancer, skin cancer, rectal cancer, osteosarcoma, multiple myeloma, glioma, ovarian cancer, pancreatic cancer, cervical cancer, endometrial cancer, thyroid cancer, laryngeal cancer, testicular cancer, mesothelioma, acute myeloid leukemia, chronic myeloid leukemia, acute lymphoblastic leukemia, chronic lymphoblastic leukemia, brain tumor, neuroblastoma, retinoblastoma, head and neck cancer, salivary gland cancer, and lymphoma.

20. A transformed cell expressing the fusion protein according to claim 1.

21. A method for treating or preventing cancer, comprising administering the fusion protein according to claim 1 to a subject in need of cancer treatment or cancer prevention.

22. (canceled)

23. (canceled)
